(19) United States
(12) Patent Application Publication
(10) Pub. No.: US 2018/0179274 A1
(43) Pub. Date: Jun. 28, 2018

(54) PROTEINS COMPRISING A MUTATED LAIR-1 FRAGMENT AND USES THEREOF

(71) Applicant: Institute for Research in Biomedicine, Bellinzona (CH)

(72) Inventors: Antonio Lanzavecchia, Porza (CH); Joshua Tan, Selangor (MY); Abdi Abdirahman, Kilifi (KE); Kathrin Pieper, Giubiasco (CH); Luca Piccoli, Bellinzona (CH); Peter Charles Bull, Cambridge (GB)

(21) Appl. No.: 15/738,912

(22) PCT Filed: Jun. 24, 2016
(86) PCT No.: PCT/EP2016/064751

§ 371 Date: Dec. 21, 2017

(30) Foreign Application Priority Data
Jun. 26, 2015 (EP) PCT/EP2015/001291
Dec. 22, 2015 (EP) PCT/EP2015/002597

(51) Int. Cl. C07K 16/20; A61P 33/02; G01N 33/569; C07K 14/705

(52) U.S. Cl. CPC C07K 16/205; A61P 33/02; G01N 33/56905; C07K 14/70503; G01N 2800/26; C07K 2317/21; C07K 2319/30; C07K 2317/33; G01N 2333/445; C07K 2317/565

(57) ABSTRACT

The present invention provides a protein comprising a mutated LAIR-1 fragment, which broadly binds to erythrocytes infected with Plasmodium falciparum. The protein comprising the mutated LAIR-1 fragment may be useful in the prevention and/or treatment of malaria. The present invention furthermore provides a nucleic acid encoding a mutated LAIR-1 fragment, a vector comprising such a nucleic acid as well as a respective pharmaceutical composition.

The present invention relates to the field of malaria medication, in particular to molecules binding to Plasmodium falciparum surface antigens.

The virulence of Plasmodium falciparum and other Plasmodia that cause malaria is attributed to the adhesion of infected erythrocytes to the vascular endothelium or to uninfected erythrocytes to form rosettes. The key to the survival of P. falciparum in the human host is its ability to undergo antigenic variation, by switching expression among protein variants encoded by multigene families, such as var, rif and stevor. About 60 var and 150 rif genes are clonally expressed by P. falciparum and encode a diverse and polymorphic set of molecules displayed on the surface of infected erythrocytes that mediate adhesion to different substrates. It is well established that the antibody response to P. falciparum-infected erythrocytes protects from lethal disease and, consequently, the discovery of specific antibodies and conserved antigens has practical relevance.

In particular, surface antigens of P. falciparum-infected erythrocytes were suggested as immune targets (for review see Chan, J.-A. et al., 2014, Cell. Mol. Life Sci. 71:3633-3657). Surface antigens of infected erythrocytes (IEs), which are also known as “variant surface antigens” or “VSA”, include PfEMP1 (P. falciparum erythrocyte membrane protein 1), RIFIN (repetitive interspersed family proteins), STEVOR (sub-telomeric variable open reading frame proteins) and SURFIN (surface-associated interspersed gene family proteins), whereby the most important immune target appeared to be PfEMP1, which is a major ligand for vascular adhesion and sequestration of IEs. Studies are beginning to identify specific variants of PfEMP1 linked to disease pathogenesis that may be suitable for vaccine development, but overcoming antigenic diversity in PfEMP1 remains a major challenge (for review see Chan, J.-A. et al., 2014, Cell. Mol. Life Sci. 71:3633-3657).

The RIFINSs, another family of antigens found on the surface of IEs, represent the largest family of antigenically variable molecules in P. falciparum. These polypeptides are encoded by 150 rif genes whose expression is upregulated in rosetting parasites. It has been recently shown that RIFINs bind preferentially to erythrocytes of blood group A to form large rosettes and to mediate vascular sequestration of IEs, indicating that they may play an important role in the development of severe malaria (Goel S. et al., 2015, Nat Med. 21(4):314-7).

Recently, there has been considerable technological progress for the isolation of broadly neutralizing human monoclonal antiviral antibodies against highly variable pathogens, such as HIV-1 and influenza virus. These antibodies can be used for passive immunotherapy but also to drive the design of immunogens capable of inducing antibodies of the same type in active vaccination (Burton D. R. et al., Cell Host Microbe, 2012, Oct. 18; 12(4):396-407). However, in spite of these successes, there is little expectation that it would be possible to find antibodies capable of recognizing the huge number of different P. falciparum strains that can infect erythrocytes, considering the extensive polymorphism and the large number of surface molecules. Similarly, it has been difficult so far to identify a structural basis for the design of a vaccine capable of eliciting antibodies that can protect against the highly variable P. falciparum strains.

In view of the above, it is the object of the present invention to overcome the drawbacks in the malaria field as outlined above. In particular, it is the object of the present invention to provide unique antigen-binding polypeptides that bind broadly to malaria-infected erythrocytes (equivalent to broadly virus neutralizing antibodies). Moreover, it is an object of the present invention to provide antigen-binding polypeptides, which opsonize infected erythrocytes, prevent vascular sequestration of IEs and, thus, favor the elimination of IEs in vivo, and prevent development of severe malaria.

This object is achieved by means of the subject-matter set out below and in the appended claims.

Although the present invention is described in detail below, it is to be understood that this invention is not limited to the particular methodologies, protocols and reagents described herein as these may vary. It is also to be understood that the terminology used herein is not intended to limit the scope of the present invention which will be limited only by the appended claims. Unless defined otherwise, all technical and scientific terms used herein have the same meanings as commonly understood by one of ordinary skill in the art.

In the following, the elements of the present invention will be described. These elements may be listed with specific embodiments, however, it should be understood that they may be combined in any manner and in any number to create additional embodiments. The variously described examples and preferred embodiments should not be construed to limit the present invention to only the explicitly described embodiments. This description should be understood to support and encompass embodiments which combine the explicitly described embodiments with any number of the disclosed and/or preferred elements. Furthermore, any permutations and combinations of all described elements in this application should be considered disclosed by the description of the present application unless the context indicates otherwise.

Throughout this specification and the claims which follow, unless the context requires otherwise, the term “comprise”, and variations such as “comprises” and “comprising”, will be understood to imply the inclusion of a stated member, integer or step but not the exclusion of any other non-stated member, integer or step. The term “consist of” is a particular embodiment of the term “comprise”, wherein any other non-stated member, integer or step is excluded. In the context of the present invention, the term “comprise” encompasses the term “consist of”. The term “comprising” thus encompasses “including” as well as “consisting” e.g., a composition “comprising” X may consist exclusively of X or may include something additional e.g., X+Y.

The terms “a” and “an” and “the” and similar reference used in the context of describing the invention (especially in the context of the claims) are to be construed to cover both the singular and the plural, unless otherwise indicated herein or clearly contradicted by context.

Recitation of ranges of values herein is merely intended to serve as a shorthand method of referring individually to each separate value falling within the range. Unless otherwise indicated herein, each individual value is incorporated into the specification as if it were individually recited herein. No language in the specification should be construed as indicating any non-claimed element essential to the practice of the invention.

The word “substantially” does not exclude “completely” e.g., a composition which is “substantially free” from Y may be completely free from Y. Where necessary, the word “substantially” may be omitted from the definition of the invention.

The term “about” in relation to a numerical value x means x±10%.

The term “disease” as used herein is intended to be generally synonymous, and is used interchangeably with, the terms “disorder” and “condition” (as in medical condition), in that all reflect an abnormal condition of the human or animal body or of one of its parts that impairs normal functioning, is typically manifested by distinguishing signs and symptoms, and causes the human or animal to have a reduced duration and/or quality of life.

As used herein, reference to “treatment” of a subject or patient is intended to include prevention, prophylaxis, attenuation, amelioration and therapy. The terms “subject” or “patient” are used interchangeably herein to mean all mammals including humans. Examples of subjects include humans, cows, dogs, cats, horses, goats, sheep, pigs, and rabbits. Preferably, the subject or patient is a human.

As used herein, the terms “peptide”, “polypeptide”, and “protein” and variations of these terms refer to a molecule, in particular a peptide, oligopeptide, polypeptide or protein including fusion protein, respectively, comprising at least two amino acids joined to each other by a normal peptide bond, or by a modified peptide bond, such as for example in the cases of isosteric peptides. For example, a “classical” peptide, polypeptide or protein is typically composed of amino acids selected from the 20 amino acids defined by the genetic code, linked to each other by a normal peptide bond. A peptide, polypeptide or protein can be composed of L-amino acids and/or D-amino acids. Preferably, a peptide, polypeptide or protein is either (entirely) composed of L-amino acids or (entirely) of D-amino acids, thereby forming “retro-inverso peptide sequences”. The term “retro-inverso (peptide) sequences” refers to an isomer of a linear peptide sequence in which the direction of the sequence is reversed and the chirality of each amino acid residue is inverted (see e.g. Jameson et al., Nature, 368,744-746 (1994); Brady et al, Nature, 368,692-693 (1994)). In particular, the terms “peptide”, “polypeptide”, “protein” also include “peptidomimetics” which are defined as peptide analogs containing non-peptidic structural elements, which peptides are capable of mimicking or antagonizing the biological action(s) of a natural parent peptide. A peptidomimetic lacks classical peptide characteristics such as enzymatically scissile peptide bonds. In particular, a peptide, polypeptide or protein may comprise amino acids other than the 20 amino acids defined by the genetic code in addition to these amino acids, or it can be composed of amino acids other than the 20 amino acids defined by the genetic code. In particular, a peptide, polypeptide or protein in the context of the present invention can equally be composed of amino acids modified by natural processes, such as post-translational maturation processes or by chemical processes, which are well known to a person skilled in the art. Such modifications are fully detailed in the literature. These modifications can appear anywhere in the polypeptide: in the peptide skeleton, in the amino acid chain or even at the carboxy- or amino-terminal ends. In particular, a peptide or polypeptide can be branched following an ubiquitination or be cyclic with or without branching. This type of modification can be the result of natural or synthetic post-translational processes that are well known to a person skilled in the art. The terms “peptide”, “polypeptide”, “protein” in the context of the present invention in particular also include modified peptides, polypeptides and proteins. For example, peptide, polypeptide or protein modifications can include acetylation, acylation, ADP-ribosylation, amidation, covalent fixation of a nucleotide or of a nucleotide derivative, covalent fixation of a lipid or of a lipidic derivative, the covalent fixation of a phosphatidylinositol, covalent or non-covalent cross-linking, cyclization, disulfide bond formation, demethylation, glycosylation including pegylation, hydroxylation, iodization, methylation, myristoylation, oxidation, proteolytic processes, phosphorylation, prenylation, racemization, seneloylation, sulfatation, amino acid addition such as arginylation or ubiquitination. Such modifications are fully detailed in the literature (Proteins Structure and Molecular Properties (1993) 2nd Ed., T. E. Creighton, New York ; Post-translational Covalent Modifications of Proteins (1983) B. C. Johnson, Ed., Academic Press, New York ; Seifter et al. (1990) Analysis for protein modifications and nonprotein cofactors, Meth. Enzymol. 182: 626-646 and Rattan et al., (1992) Protein Synthesis: Post-translational Modifications and Aging, Ann NY Acad Sci, 663: 48-62). Accordingly, the terms “peptide”, “polypeptide”, “protein” preferably include for example lipopeptides, lipoproteins, glycopeptides, glycoproteins and the like.

As used herein a “(poly)peptide” comprises a single chain of amino acid monomers linked by peptide bonds as explained above. A “protein”, as used herein, comprises one or more, e.g. 1, 2, 3, 4, 5, 6, 7, 8, 9 or 10 (poly)peptides, i.e. one or more chains of amino acid monomers linked by peptide bonds as explained above. Preferably, a protein according to the present invention comprises 1, 2, 3, or 4 polypeptides.

The term “recombinant protein”, as used herein, refers to any protein which is prepared, expressed, created or isolated by recombinant means, and which is not naturally occurring.

As used herein, the terms “nucleic acid”, “nucleic acid molecule” and “polynucleotide” are used interchangeably and are intended to include DNA molecules and RNA molecules. A nucleic acid molecule may be single-stranded or double-stranded, but preferably is double-stranded DNA.

As used herein, the terms “cell,” “cell line,” and “cell culture” are used interchangeably and all such designations include progeny. Thus, the words “transformants” and “transformed cells” include the primary subject cell and cultures derived therefrom without regard for the number of transfers. It is also understood that all progeny may not be precisely identical in DNA content, clue to deliberate or inadvertent mutations. Variant progeny that have the same function or biological activity as screened for in the originally transformed cell are included. Where distinct designations are intended, it will be clear from the context.

Doses are often expressed in relation to the bodyweight. Thus, a dose which is expressed as [g, mg, or other unit]/kg (or g, mg etc.) usually refers to [g, mg, or other unit] “per kg (or g, mg etc.) bodyweight”, even if the term “bodyweight” is not explicitly mentioned.

The terms “binding” and, in particular, “specifically binding” and similar reference does not encompass non-specific sticking.

As used herein, the term “sequence variant” refers to any alteration in a reference sequence, whereby a reference sequence is any of the sequences listed herein, i.e. SEQ ID NO: 1 to SEQ ID NO: 642. Thus, the term “sequence variant” includes nucleotide sequence variants and amino acid sequence variants. In particular, in a “sequence variant” the functionality (of the reference sequence) is preserved, i.e. the sequence variant is functional (also referred to as “functional sequence variant”). A “sequence variant” as used herein typically has a sequence which is at least 70% identical to the reference sequence, preferably at least 80% identical to the reference sequence, more preferably at least 90% identical, even more preferably at least 95% identical, and particularly preferably at least 99% identical to the reference sequence.

Sequence identity is usually calculated with regard to the full length of the reference sequence (i.e. the sequence recited in the application). Percentage identity, as referred to herein, can be determined, for example, using BLAST using the default parameters specified by the NCBI (the National Center for Biotechnology Information; http://www.ncbi.nlm.nih.gov/) [Blosum 62 matrix; gap open penalty=11 and gap extension penalty=1].

A “sequence variant” in the context of a nucleotide sequence has an altered sequence in which one or more of the nucleotides in the reference sequence is deleted, or substituted, or one or more nucleotides are inserted into the sequence of the reference nucleotide sequence. Nucleotides are referred to herein by the standard one-letter designation (A, C, G, or T). Due to the degeneracy of the genetic code, a “sequence variant” of a nucleic acid (nucleotide) sequence can either result in a change in the respective reference amino acid sequence, i.e. in a “sequence variant” of the respective amino acid sequence or not. Preferred sequence variants are such nucleotide sequence variants, which do not result in amino acid sequence variants (silent mutations), but other non-silent mutations are within the scope as well, in particular mutant nucleotide sequences, which result in an amino acid sequence, which is at least 70% identical to the reference sequence, preferably at least 80% identical to the reference sequence, more preferably at least 90% identical, even more preferably at least 95% identical, and particularly preferably at least 99% identical to the reference sequence.

A “sequence variant” in the context of an amino acid has an altered sequence in which one or more of the amino acids in the reference sequence is deleted or substituted, or one or more amino acids are inserted into the sequence of the reference amino acid sequence. As a result of the alterations, the amino acid sequence variant has an amino acid sequence which is at least 70% identical to the reference sequence, preferably at least 80% identical to the reference sequence, more preferably at least 90% identical, even more preferably at least 95% identical, and particularly preferably at least 99% identical to the reference sequence. Variant sequences which are at least 90% identical have no more than 10 alterations, i.e. any combination of deletions, insertions or substitutions, per 100 amino acids of the reference sequence.

In the context of peptides/proteins, a “linear sequence” or a “sequence” is the order of amino acids in a peptide/protein in an amino to carboxyl terminal direction in which residues that neighbor each other in the sequence are contiguous in the primary structure of the peptide/protein.

While it is possible to have non-conservative amino acid substitutions in a “sequence variant”, it is preferred in a “sequence variant” that the substitutions are conservative amino acid substitutions, in which the substituting amino acid has similar structural and/or chemical properties as the corresponding substituted amino acid (i.e. the amino acid in the original sequence which was substituted). By way of example, conservative amino acid substitutions involve substitution of one aliphatic or hydrophobic amino acid, e.g. alanine, valine, leucine and isoleucine, with another; substitution of one hydroxyl-containing amino acid, e.g. serine and threonine, with another; substitution of one acidic residue, e.g. glutamic acid or aspartic acid, with another; replacement of one amide-containing residue, e.g. asparagine and glutamine, with another; replacement of one aromatic residue, e.g. phenylalanine and tyrosine, with another; replacement of one basic residue, e.g. lysine, arginine and histidine, with another; and replacement of one small amino acid, e.g., alanine, serine, threonine, methionine, and glycine, with another.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include the fusion to the N- or C-terminus of an amino acid sequence to a reporter molecule or an enzyme.

Importantly, the sequence variants are functional sequence variants, i.e. the alterations in the sequence variants do not abolish the functionality of the respective reference sequence, in the present case, e.g., the functionality of a mutated LAIR-1 (Leukocyte-associated immunoglobulin-like receptor) fragment according to the present invention to bind to the same epitope/site of a P. falciparum surface antigen, in particular a RlFIN, expressed on the surface of an IE or on a parasite, and/or to sufficiently neutralize infection with P. falciparum. Guidance in determining which nucleotides and amino acid residues, respectively, may be substituted, inserted or deleted without abolishing such functionality are found by using computer programs well known in the art.

As used herein, a nucleic acid sequence or an amino acid sequence “derived from” a designated nucleic acid, peptide, polypeptide or protein refers to the origin of the polypeptide. Preferably, the nucleic acid sequence or amino acid sequence which is derived from a particular sequence has an amino acid sequence that is essentially identical to that sequence or a portion thereof, from which it is derived, whereby “essentially identical” includes sequence variants as defined above. Preferably, the nucleic acid sequence or amino acid sequence which is derived from a particular peptide or protein, is derived from the corresponding domain in the particular peptide or protein. Thereby, “corresponding” refers in particular to the same functionality. For example, an “extracellular domain” corresponds to another “extracellular domain” (of another protein), or a “transmembrane domain” corresponds to another “transmembrane domain” (of another protein). “Corresponding” parts of peptides, proteins and nucleic acids are thus easily identifiable to one of ordinary skill in the art. Likewise, sequences “derived from” other sequence are usually easily identifiable to one of ordinary skill in the art as having its origin in the sequence.

Preferably, a nucleic acid sequence or an amino acid sequence derived from another nucleic acid, peptide, polypeptide or protein may be identical to the starting nucleic acid, peptide, polypeptide or protein (from which it is derived). However, a nucleic acid sequence or an amino acid sequence derived from another nucleic acid, peptide, polypeptide or protein may also have one or more mutations relative to the starting nucleic acid, peptide, polypeptide or protein (from which it is derived), in particular a nucleic acid sequence or an amino acid sequence derived from another nucleic acid, peptide, polypeptide or protein may be a functional sequence variant as described above of the starting nucleic acid, peptide, polypeptide or protein (from which it is derived). For example, in a peptide/protein one or more amino acid residues may be substituted with other amino acid residues or one or more amino acid residue insertions or deletions may occur.

As used herein, the term “mutation” relates to a change in the nucleic acid sequence and/or in the amino acid sequence in comparison to a reference sequence, e.g. a corresponding genomic sequence. A mutation, e.g. in comparison to a genomic sequence, may be, for example, a (naturally occurring) somatic mutation, a spontaneous mutation, an induced mutation, e.g. induced by enzymes, chemicals or radiation, or a mutation obtained by site-directed mutagenesis (molecular biology methods for making specific and intentional changes in the nucleic acid sequence and/or in the amino acid sequence). Thus, the terms “mutation” or “mutating” shall be understood to also include physically making a mutation, e.g. in a nucleic acid sequence or in an amino acid sequence. A mutation includes substitution, deletion and insertion of one or more nucleotides or amino acids as well as inversion of several successive nucleotides or amino acids. To achieve a mutation in an amino acid sequence, preferably a mutation may be introduced into the nucleotide sequence encoding said amino acid sequence in order to express a (recombinant) mutated polypeptide. A mutation may be achieved e.g., by altering, e.g., by site-directed mutagenesis, a codon of a nucleic acid molecule encoding one amino acid to result in a codon encoding a different amino acid, or by synthesizing a sequence variant, e.g., by knowing the nucleotide sequence of a nucleic acid molecule encoding a polypeptide and by designing the synthesis of a nucleic acid molecule comprising a nucleotide sequence encoding a variant of the polypeptide without the need for mutating one or more nucleotides of a nucleic acid molecule.

Several documents are cited throughout the text of this specification. Each of the documents cited herein (including all patents, patent applications, scientific publications, manufacturer's specifications, instructions, etc.), whether supra or infra, are hereby incorporated by reference in their entirety. Nothing herein is to be construed as an admission that the invention is not entitled to antedate such disclosure by virtue of prior invention.

It is to be understood that this invention is not limited to the particular methodology, protocols and reagents described herein as these may vary. It is also to be understood that the terminology used herein is for the purpose of describing particular embodiments only, and is not intended to limit the scope of the present invention which will be limited only by the appended claims. Unless defined otherwise, all technical and scientific terms used herein have the same meanings as commonly understood by one of ordinary skill in the art.

The present invention is based, amongst other findings, on the surprising finding that a fragment of LAIR-1, which is about 100 amino acids long and carries at least one mutation as described below and in the appended claims, is able to bind to erythrocytes infected with Plasmodium falciparum. Surprisingly, this mutated LAIR-1 fragment binds broadly to malaria-infected erythrocytes, i.e. it binds to erythrocytes infected by different P. falciparum strains. Thus, the mutated LAIR-1 fragment can be used to produce a potent immunoadhesin.

Protein Comprising a Mutated LAIR-7 Fragment

In a first aspect the present invention provides a protein comprising or consisting of at least amino acids 67 to 107 of native human LAIR-1, wherein said LAIR-1 fragment comprises:
a. 1, 2, 3, 4, or 5 mutations in comparison to native human LAIR-1 at one or more of the following five positions: T67, N69, A77, P106, and P107; and
b. optionally, one or more further mutations at a position different from T67, N69, A77, P106, and P107 in comparison to native human LAIR-1,

wherein said LAIR-1 fragment shows at least 70% amino acid sequence identity to amino acids 67 to 107 of native human LAIR-1 (SEQ ID NO: 9).

In other words, the protein according to the present invention comprises (or consists of) a LAIR-1 fragment consisting of at least amino acids 67 to 107 of native human LAIR-1, wherein said LAIR-1 fragment comprises:
(i) 1, 2, 3, 4, or 5 mutations in comparison to native human LAIR-1, at one or more of the following five positions: T67, N69, A77, P106, and P107; and
(ii) optionally, one or more further mutations at a position different from T67, N69, A77, P106, and P107 in comparison to native human LAIR-1,

wherein said LAIR-1 fragment shows at least 70% amino acid sequence identity to amino acids 67 to 107 of native human LAIR-1 (SEQ ID NO: 9).

Mutated LAIR-1 Fragment

Thus, the protein according to the present invention comprising (or consisting of) the mutated LAIR-1 fragment as described above, comprises at least the 1, 2, 3, 4, or 5 mutations at one or more of the following five positions: T67, N69, A77, P106, and P107. One or more of these mutations enable binding of the protein according to the present invention to erythrocytes infected with P. falciparum, in particular to a surface antigen thereof. Accordingly, such a protein according to the present invention may be used in diagnosis, prevention and/or treatment of malaria.

Optionally, the protein according to the present invention comprising (or consisting of) the mutated LAIR-1 fragment as described above may comprise further mutations at positions different from T67, N69, A77, P106, and P107 (i.e. in addition to one or more mutation(s) at one or more of the following five positions: T67, N69, A77, P106, and P107), with the proviso that the LAIR-1 fragment shows at least 70% amino acid sequence identity to amino acids 67 to 107 of native human LAIR-1 (SEQ ID NO: 9). Thus, one or more of such further mutations may occur in the LAIR-1 fragment comprised by the protein according to the present invention.

The above described mutations in the protein according to the present invention (i.e. the mutations at positions T67, N69, A77, P106, and P107 and the mutations at positions different from T67, N69, A77, P106, and P107) may be a substitution, a deletion and/or an insertion of one or more amino acids and/or an inversion of more than one subsequent amino acids. Of these different kinds of mutations, in the protein according to the present invention one or more deletion mutations and/or one or more substitution mutations are preferred. In other words, that the above described mutations in the protein according to the present invention (i.e. the mutations at positions T67, N69, A77, P106, and P107 and the mutations at positions different from T67, N69, A77, P106, and P107 in the LAIR-1 fragment) are preferably deletion and/or substitution mutations. More preferably, the above described mutations in the protein according to the present invention (i.e. the mutations at positions T67, N69, A77, P106, and P107 and the mutations at positions different from T67, N69, A77, P106, and P107 in the LAIR-1 fragment) are substitution mutations.

Amino acid sequence identity may be calculated as described above. In particular, the expression “LAIR-1 fragment” refers to fragment of the protein according to the present invention (i.e. to a stretch of consecutive amino acids linked in particular by a peptide bond, which is comprised by the protein according to the present invention), which shows at least 70% amino acid sequence identity to amino acids 67 to 107 of native human LAIR-1 as described below (SEQ ID NO: 9). Thus, such a “LAIR-1 fragment” in particular comprises no more than 12 amino acid mutations (in total, i.e. comprising the 1-5 mutation(s) at any of positions T67, N69, A77, P106, and P107 and the mutation(s) at other position(s)) in comparison to amino acids 67 to 107 of native human LAIR-1 (i.e. in comparison to an amino acid sequence according to SEQ ID NO: 9, which has a length of 41 amino acids).

Preferably, the mutated LAIR-1 fragment comprised by the protein according to the present invention shows at least 75% amino acid sequence identity to amino acids 67 to 107 of native human LAIR-1 (SEQ ID NO: 9). In other words, the mutated LAIR-1 fragment comprised by the protein according to the present invention comprises preferably no more than 10 amino acid mutations in comparison to amino acids 67 to 107 of native human LAIR-1 (i.e. in comparison to an amino acid sequence according to SEQ ID NO: 9, which has a length of 41 amino acids).

More preferably, the mutated LAIR-1 fragment comprised by the protein according to the present invention shows at least 80% amino acid sequence identity to amino acids 67 to 107 of native human LAIR-1 (SEQ ID NO: 9). In other words, the mutated LAIR-1 fragment comprised by the protein according to the present invention more preferably comprises no more than 8 amino acid mutations in comparison to amino acids 67 to 107 of native human LAIR-1 (i.e. in comparison to an amino acid sequence according to SEQ ID NO: 9, which has a length of 41 amino acids).

Even more preferably, the mutated LAIR-1 fragment comprised by the protein according to the present invention shows at least 85% amino acid sequence identity to amino acids 67 to 107 of native human LAIR-1 (SEQ ID NO: 9). In other words, the mutated LAIR-1 fragment comprised by the protein according to the present invention even more preferably comprises no more than 6 amino acid mutations in comparison to amino acids 67 to 107 of native human LAIR-1 (i.e. in comparison to an amino acid sequence according to SEQ ID NO: 9, which has a length of 41 amino acids).

Particularly preferably, the mutated LAIR-1 fragment comprised by the protein according to the present invention shows at least 87% amino acid sequence identity to amino acids 67 to 107 of native human LAIR-1 (SEQ ID NO: 9). In other words, the mutated LAIR-1 fragment comprised by the protein according to the present invention particularly preferably comprises no more than 5 amino acid mutations in comparison to amino acids 67 to 107 of native human LAIR-1 (i.e. in comparison to an amino acid sequence according to SEQ ID NO: 9, which has a length of 41 amino acids).

The amino acid used for a insertion or substitution mutation, preferably for a substitution mutation, in particular the amino acid substituting one of T67, N69, A77, P106, and P107, may be any amino acid, preferably a proteinogenic amino acid, i.e. an amino acid, which is able to make up a protein. Thus, the amino acid used for a substitution mutation, in particular the amino acid substituting one of T67, N69, A77, P106, and P107, is preferably selected from the 20 amino acids, which are directly encoded by the genetic code, namely, alanine (A), arginine (R), asparagine (N), aspartic acid (D), cysteine (C), glutamic acid (E), glutamine (Q), glycine (G), histidine (H), isoleucine (I), leucine (L), lysine (K), methionine (M), phenylalanine (F), proline (P), serine (S), threonine (T), tryptophan (W), tyrosine (Y), and valine (V). Needless to say, the amino acid substituting one of T67, N69, A77, P106, and P107 must be different from the amino acid which is originally found in this position, i.e. the amino acid substituting T67 is not threonine, the amino acid substituting N69 is not asparagine, the amino acid substituting A77 is not alanine, the amino acid substituting P106 is not proline, and the amino acid substituting P107 is not proline.

As described above, the optional one or more further mutations at a position different from T67, N69, A77, P106, and P107 are preferably a deletion and/or a substituation, whereby a substituation is more preferred. For an amino acid substitution at a position different from T67, N69, A77, P106, and P107 it is preferred that such a substitution is a conservative amino acid substitution. In a conservative amino acid substitution the substituting amino acid has similar structural and/or chemical properties as the corresponding substituted amino acid (i.e. the amino acid in the original sequence which was substituted). By way of example, conservative amino acid substitutions involve substitution of one aliphatic or hydrophobic amino acid, e.g. alanine, valine, leucine and isoleucine, with another; substitution of one hydoxyl-containing amino acid, e.g. serine and threonine, with another; substitution of one acidic residue, e.g. glutamic acid or aspartic acid, with another; substitution of one amide-containing residue, e.g. asparagine and glutamine, with another; substitution of one aromatic residue, e.g. phenylalanine and tyrosine, with another; substitution of one basic residue, e.g. lysine, arginine and histidine, with another; and substitution of one small amino acid, e.g., alanine, serine, threonine, methionine, and glycine, with another.

As used herein, the term “LAIR-1” refers to the protein “Leukocyte-associated immunoglobulin-like receptor 1”, which is also known as CD305. LAIR-1 is an inhibitory receptor widely expressed throughout the immune system, i.e. on peripheral mononuclear cells, including NK cells, T cells, and B cells. LAIR-1 regulates the immune response, in particular to prevent lysis of cells recognized as self. Collagens and C1q were found to be high-affinity functional ligands of LAIR-1.

LAIR-1 was implicated in various functions, including reduction of the increase of intracellular calcium evoked by B-cell receptor ligation; modulation of cytokine production in CD4+ T-cells, thereby down-regulating IL-2 and IFN-gamma production while inducing secretion of transforming growth factor-beta; down-regulation of IgG and IgE production in B-cells as well as IL-8, IL-10 and TNF secretion; inhibition of proliferation and induction of apoptosis in myeloid leukemia cell lines as well as prevention of nuclear translocation of NF-kappa-B p65 subunit/RELA and phosphorylation of I-kappa-B alpha/CHUK in these cells; and inhibition of differentiation of peripheral blood precursors towards dendritic cells. Activation by Tyr phosphorylation results in recruitment and activation of the phosphatases PTPN6 and PTPN11. A more detailed overview over the various functions of LAIR-1 is provided by Meyaard L, 2008, J Leukoc Biol. 83(4):799-803.

The gene LAIR1, which encodes the protein LAIR-1, is a member of both the immunoglobulin superfamily and the leukocyte-associated inhibitory receptor family. LAIR1 consists of 10 exons and shows considerable homology to LAIR2. The LAIR-2 gene encodes a protein hLAIR-2 that is about 84% homologous to hLAIR-1 but lacks a transmembrane and an intracellular domain (cf. Meyaard L., 2008, J Leukoc Biol. 83(4):799-803). In particular, the mutated LAIR-1 fragment comprised by the protein according to the present invention may thus also be a corresponding “mutated LAIR-2 fragment”, which is mutated accordingly, i.e. in respect to the 1, 2, 3, 4, or 5 mutations at one or more of the five positions corresponding to T67, N69, A77, P106, and P107 in native human LAIR-1.

Human LAIR-1 is a type I transmembrane glycoprotein of 287 amino acids containing a single extracellular C2-type Ig-like domain and two ITIMs in its cytoplasmic tail. An ITIM is an immunoreceptor tyrosine-based inhibition motif (ITIM), which is a conserved sequence of amino acids (S/IN/LxYxxIN/L) that is found in the cytoplasmic tails of many inhibitory receptors of the immune system. LAIR-1 is structurally related to several other inhibitory Ig superfamily members localized to the leukocyte receptor complex (LRC) on human chromosome 19q13.4, suggesting that these molecules have evolved from a common ancestral gene.

Of the 287 amino acids of human native LAIR-1, in the order from N- to C-terminus, amino acids 1 to 21 represent a signal peptide, amino acids 22 to 165 represent an extracellular domain, amino acids 166 to 186 represent a transmembrane domain, and amino acids 187 to 287 represent a cytoplasmic domain. In mature LAIR-1, the signal peptide is typically removed, i.e. mature LAIR-1 typically comprises amino acids 22 to 287.

Several different splice variants of the LAIR-family have been cloned. LAIR-1 b lack 17 amino acids in the stalk region between the transmembrane domain and Ig-like domain as compared with the full-length LAIR-1a, which may affect their glycosylation (for review see Meyaard L., 2008, J Leukoc Biol. 83(4):799-803). LAIR-1a and LAIR-1b might be differentially expressed in NK and T cells, but the relevance of this has not been studied extensively. LAIR-1c is identical to LAIR-1b except for a single amino acid deletion in the extracellular domain, namely, one of the glutamic acid residues at positions E23 and E24 of LAIR-1 a, LAIR-1b, and LAIR-1d is deleted in LAIR-1c. LAIR-1d lacks part of the intracellular tail (for review see Meyaard L., 2008, J Leukoc Biol. 83(4):799-803). Genebank accession codes of the cloned cDNAs are: AF013249 (human LAIR-1a), AF109683 (human LAIR-1b), AF251509 (human LAIR-1 c), AF251510 (human LAIR-1d).

In the following, the sequences of the four human LAIR-1 splice variants are provided (amino acid sequences and cDNA sequences). The five amino acid positions T67, N69, A77, P106, and P107, which are particularly relevant for the mutations in the LAIR-1 fragment according to the present invention, are shown in bold.

hLAIR-1a amino acid sequence, cf. GenBank
accession code AF013249 - “translated protein”:

SEQ ID NO: 1

MSPHPTALLGLVLCLAQTIHTQEEDLPRPSISAEPGTVIPLGSHVTFVCR
GPVGVQTERLERESRSTYNDTEDVSQASPSESEARFRIDSVSEGNAGPYR
CIYYKPPKWSEQSDYLELLVKETSGGPDSPDTEPGSSAGPTQRPSDNSHN
EHAPASQGLKAEHLYILIGVSVVFLFCLLLLVLFCLHRQNQIKQGPPRSK
DEEQKPQQRPDLAVDVLERTADKATVNGLPEKDRETDTSALAAGSSQEVT
YAQLDHWALTQRTARAVSPQSTKPMAESITYAAVARH
hLAIR1a nucleotide sequence, cf. GenBank accession
code AF013249 - “CDS”:

SEQ ID NO: 2

atgtctccccaccccaccgccctcctgggcctagtgctctgcctggccca
gaccatccacacgcaggaggaagatctgcccagaccctccatctcggctg
agccaggcaccgtgatccccctggggagccatgtgactttcgtgtgccgg
ggcccggttggggttcaaacattccgcctggagagggagagtagatccac
atacaatgatactgaagatgtgtctcaagctagtccatctgagtcagagg
ccagattccgcattgactcagtaagtgaaggaaatgccgggccttatcgc
tgcatctattataagccccctaaatggtctgagcagagtgactacctgga
gctgctggtgaaagaaacctctggaggcccggactccccggacacagagc
ccggctcctcagctggacccacgcagaggccgtcggacaacagtcacaat
gagcatgcacctgcttcccaaggcctgaaagctgagcatctgtatattct
catcggggtctcagtggtcttcctcttctgtctcctcctcctggtcctct
tctgcctccatcgccagaatcagataaagcaggggccccccagaagcaag
gacgaggagcagaagccacagcagaggcctgacctggctgttgatgttct
agagaggacagcagacaaggccacagtcaatggacttcctgagaaggaca
gagagacggacacctcggccctggctgcagggagttcccaggaggtgacg
tatgctcagctggaccactgggccctcacacagaggacagcccgggctgt
gtccccacagtccacaaagcccatggccgagtccatcacgtatgcagccg
ttgccagacactga
hLAIR-1b amino acid sequence, cf. GenBank
accession code AF109683 - “translated protein”:

SEQ ID NO: 3

MSPHPTALLGLVLCLAQTIHTQEEDLPRPSISAEPGTVIPLGSHVTFVCR
GPVGVQTFRLERESRSTYNDTEDVSQASPSESEARFRIDSVSEGNAGPYR
CIYYKPPKWSEQSDYLELLVKGPTQRPSDNSHNEHAPASQGLKAEHLYIL
IGVSVVFLFCLLLLVLFCLHRQNQIKQGPPRSKDEEQKPQQRPDLAVDVL
ERTADKATVNGLPEKDRETDTSALAAGSSQEVTYAQLDHWALTQRTARAV
SPQSTKPMAESITYAAVARH
hLAIR1b nucleotide sequence, cf. GenBank accession
code AF109683 - “CDS”:

SEQ ID NO: 4

atgtctccccaccccaccgccctcctgggcctagtgctctgcctggccca
gaccatccacacgcaggaggaagatctgcccagaccctccatctcggctg
agccaggcaccgtgatccccctggggagccatgtgactacgtgtgccggg
gcccggttggggttcaaacattccgcctggagagggagagtagatccaca
tacaatgatactgaagatgtgtctcaagctagtccatctgagtcagaggc
cagattccgcattgactcagtaagtgaaggaaatgccgggccttatcgct
gcatctattataagccccctaaatggtctgagcagagtgactacctggag
ctgctggtgaaaggacccacgcagaggccgtcggacaacagtcacaatga
gcatgcacctgcttcccaaggcctgaaagctgagcatctgtatattctca
tcggggtctcagtggtcttcctcttctgtctcctcctcctggtcctcttc
tgcctccatcgccagaatcagataaagcaggggccccccagaagcaagga
cgaggagcagaagccacagcagaggcctgacctggctgttgatgttctag
agaggacagcagacaaggccacagtcaatggacttcctgagaaggacaga
gagacggacacctcggccctggctgcagggagttcccaggaggtgacgta
tgctcagctggaccactgggccctcacacagaggacagcccgggctgtgt
ccccacagtccacaaagcccatggccgagtccatcacgtatgcagccgtt
gccagacactga
hLAIR-1c amino acid sequence, cf. GenBank
accession code AF251509 - “translated protein”:

SEQ ID NO: 5

MSPHPTALLGLVLCLAQTIHTQEDLPRPSISAEPGTVIPLGSHVTFVCRG
PVGVQTFRLERESRSTYNDTEDVSQASPSESEARFRIDSVSEGNAGPYRC
IYYKPPKWSEQSDYLELLVKGPTQRPSDNSHNEHAPASQGLKAEHLYILI
GVSVVFLFCLLLLVLFCLHRQNQIKQGPPRSKDEEQKPQQRPDLAVDVLE
RTADKATVNGLPEKDRETDTSALAAGSSQEVTYAQLDHWALTQRTARAVS
PQSTKPMAESITYAAVARH
hLAIR1c nucleotide sequence, cf. GenBank accession
code AF251509 - “CDS”:

SEQ ID NO: 6

atgtctccccaccccaccgccctcctgggcctagtgctctgcctggccca
gaccatccacacgcaggaggatctgcccagaccctccatctcggctgagc
caggcaccgtgatccccctggggagccatgtgactttcgtgtgccggggc
ccggttggggttcaaacattccgcctggagagggagagtagatccacata
caatgatactgaagatgtgtctcaagctagtccatctgagtcagaggcca
gattccgcattgactcagtaagtgaaggaaatgccgggccttatcgctgc
atctattataagccccctaaatggtctgagcagagtgactacctggagct
gctggtgaaaggacccacgcagaggccgtcggacaacagtcacaatgagc
atgcacctgcttcccaaggcctgaaagctgagcatctgtatattctcatc
ggggtctcagtggtcttcctcttctgtctcctcctcctggtcctcttctg
cctccatcgccagaatcagataaagcaggggccccccagaagcaaggacg
aggagcagaagccacagcagaggcctgacctggctgttgatgttctagag
aggacagcagacaaggccacagtcaatggacttcctgagaaggacagaga
gacggacacctcggccctggctgcagggagttcccaggaggtgacgtatg
ctcagctggaccactgggccctcacacagaggacagcccgggctgtgtcc
ccacagtccacaaagcccatggccgagtccatcacgtatgcagccgttgc
cagacactga
hLAIR-1d amino acid sequence, cf. GenBank
accession code AF251510 - “translated protein”:

SEQ ID NO: 7

MSPHPTALLGLVLCLAQTIHTQEEDLPRPSISAEPGTVIPLGSHVTFVCR
GPVGVQTFRLERESRSTYNDTEDVSQASPSESEARFRIDSVSEGNAGPYR
CIYYKPPKWSEQSDYLELLVKETSGGPDSPDTEPGSSAGPTQRPSDNSHN
EHAPASQGLKAEHLYILIGVSVVFLFCLLLLVLFCLHRQNQIKQGPPRSK
DEEQKPQQR
hLAIR1d nucleotide sequence, cf. GenBank accession
code AF251510 - “CDS”:

SEQ ID NO: 8

atgtctccccaccccaccgccctcctgggcctagtgctctgcctggccca
gaccatccacacgcaggaggaagatctgcccagaccctccatctcggctg
agccaggcaccgtgatccccctggggagccatgtgactttcgtgtgccgg
ggcccggttggggttcaaacattccgcctggagagggagagtagatccac
atacaatgatactgaagatgtgtctcaagctagtccatctgagtcagagg
ccagattccgcattgactcagtaagtgaaggaaatgccgggccttatcgc
tgcatctattataagccccctaaatggtctgagcagagtgactacctgga
gctgctggtgaaagaaacctctggaggcccggactccccggacacagagc
ccggctcctcagctggacccacgcagaggccgtcggacaacagtcacaat
gagcatgcacctgcttcccaaggcctgaaagctgagcatctgtatattct
catcggggtctcagtggtcttcctcttctgtctcctcctcctggtcctct
tctgcctccatcgccagaatcagataaagcaggggccccccagaagcaag
gacgaggagcagaagccacagcagaggtga

Of note, all of the four isoforms of human native LAIR-1 comprise the identical sequence motif according to SEQ ID NO: 9 as shown below (i.e. amino acids 67 to 107 of native human LAIR-1), which comprises the five amino acid positions at which a mutation may occur in the LAIR-1 fragment according to the present invention (shown in bold):

(SEQ ID NO: 9)

TYNDTEDVSQASPSESEARFRIDSVSEGNAGPYRCIYYKPP

This motif is shown underlined in the above amino acid sequences of the four isoforms of native human LAIR-1 (cf. SEQ ID NOs 1, 3, 5, and 7).

Of note, the positions T67, N69, A77, P106, and P107 are identical in human LAIR-1a, hLAIR-1b, and hLAIR-1d, while in hLAIR-1c (SEQ ID NO: 5) these positions are shifted—due to the deletion of one of E23 and E24—to the positions T66, N68, A76, P105, and P106. It is understood that the expressions “at one or more of the following five positions: T67, N69, A77, P106, and P107” and “at a position different from T67, N69, A77, P106, and P107” as used herein, thus refers to exactly these positions of hLAIR-1a, hLAIR-1b, and hLAIR-1d—whereas it refers to positions T66, N68, A76, P105, and P106 in hLAIR-1c.

Preferably, the LAIR-1 fragment comprised by the protein according to the present invention comprises or consists of an amino acid sequence according to SEQ ID NO: 10, as shown below, with the proviso that said LAIR-1 fragment shows at least 70% amino acid sequence identity, preferably at least 75% amino acid sequence identity, more preferably at least 80% amino acid sequence identity, even more preferably at least 85% amino acid sequence identity, and particularly preferably at least 87% amino acid sequence identity to amino acids 67 to 107 of native human LAIR-1 (SEQ ID NO: 9) in a section of the LAIR-1 fragment, which corresponds to amino acids 67 to 107 of native human LAIR-1—as described above.

SEQ ID NO: 10

X₁YX₂XXEXVXXX₃XPXXSEARFRXXSVXXGXXGXXRCXYYXX₄X₅

wherein
X is any amino acid;
X₁ is any amino acid or no amino acid; however, if X₂ is N, X₃ is A, X₄ is P and X₅ is P, then X₁ is any amino acid except T or no amino acid;
X₂ is any amino acid; however, if X₁ is T, X₃ is A, X₄ is P and X₅ is P, then X₂ is any amino acid except N;
X₃ is any amino acid; however, if X₁ is T, X₂ is N, X₄ is P and X₅ is P, then X₃ is any amino acid except A;
X₄ is any amino acid; however, if X₁ is T, X₂ is N, X₃ is A and X₅ is P, then X₄ is any amino acid except P; and
X₅ is any amino acid; however, if X₁ is T, X₂ is N, X₃ is A and X₄ is P, then X₅ is any amino acid except P.

If an amino acid is substituted in a position “X” of SEQ ID NO: 10, such a substitution is preferably a conservative substitution as described herein.

Preferably, the LAIR-1 fragment comprised by the protein according to the present invention comprises at least amino acids 50 to 110 of native human LAIR-1, more preferably at least amino acids 40 to 115 of native human LAIR-1, even more preferably at least amino acids 30 to 120 of native human LAIR-1, and particularly preferably at least amino acids 24 to 121 of native human LAIR-1.

Thus, in that particularly preferred case, wherein the LAIR-1 fragment comprised by the protein according to the present invention comprises or consists of at least amino acids 24 to 121 of native human LAIR-1, the LAIR-1 fragment comprised by the protein according to the present invention comprises or consists of the polypeptide encoded by the third exon of native human LAIR-1. Namely, the gene LAIR-1 (identifier: ENSG00000167613) is located on human chromosome 19: 54,351,384-54,370,558 reverse strand. The “third exon” of native human LAIR-1 comprises, in particular consists of, amino acids 23-120 in case of the third exon of the LAIR-1 isoform hLAIR-1c (identifier: ENSE00003486227), while the “third exon” of native human LAIR-1 comprises, in particular consists of, amino acids 24-121 in case of the third exon (identifier: ENSE00003554448) of the other LAIR-1 isoforms.

It is understood that the above positions refer to hLAIR-1a, hLAIR-1b, and hLAIR-1d, whereas in hLAIR-1c the corresponding positions are amino acids 49 to 109 (“positions 50 to 110”), amino acids 39 to 114 (“positions 40 to 115”), amino acids 29 to 119 (“positions 30 to 120”), and amino acids 23 to 120 (“positions 24 to 121”), respectively. However, the respective sequences (SEQ ID NOs 11-14) are identical as shown below.

Preferably, if such larger LAIR-1 fragments are used, the amino acid sequence identity, which is at least 70%, preferably at least 75%, more preferably at least 80%, even more preferably at least 85% and particularly preferably at least 87%, is calculated in comparison to the respective larger native human LAIR-1. Namely, for a LAIR-1 fragment comprising at least amino acids 50 to 110 of native human LAIR-1, the sequence identity is preferably calculated in comparison to amino acids 50 to 110 of native human LAIR-1 (SEQ ID NO: 11); for a LAIR-1 fragment comprising at least amino acids 40 to 115 of native human LAIR-1, the sequence identity is preferably calculated in comparison to amino acids 40 to 115 of native human LAIR-1 (SEQ ID NO: 12); for a LAIR-1 fragment comprising at least amino acids 30 to 120 of native human LAIR-1, the sequence identity is preferably calculated in comparison to amino acids 30 to 120 of native human LAIR-1 (SEQ ID NO: 13); and for a LAIR-1 fragment comprising at least amino acids 24 to 121 of native human LAIR-1, the sequence identity is preferably calculated in comparison to amino acids 24 to 121 of native human LAIR-1 (SEQ ID NO: 14).

native LAIR-1, amino acids 50-110:

SEQ ID NO: 11

RGPVGVQTFRLERESRSTYNDTEDVSQASPSESEARFRIDSVSEGNAGP
YRCIYYKPPKWS
native LAIR-1, amino acids 40-115:

SEQ ID NO: 12

PLGSHVTFVCRGPVGVQTFRLERESRSTYNDTEDVSQASPSESEARFRI
DSVSEGNAGPYRCIYYKPPKWSEQSDY
native LAIR-1, amino acids 30-120:

SEQ ID NO: 13

SISAEPGTVIPLGSHVTFVCRGPVGVQTFRLERESRSTYNDTEDVSQAS
PSESEARFRIDSVSEGNAGPYRCIYYKPPKWSEQSDYLELLV
native LAIR-1, amino acids 24-121:

SEQ ID NO: 14

EDLPRPSISAEPGTVIPLGSHVTFVCRGPVGVQTFRLERESRSTYNDTE
DVSQASPSESEARFRIDSVSEGNAGPYRCIYYKPPKWSEQSDYLELLVK

Preferably, the protein according to the present invention comprises (or consists of) a LAIR-1 fragment consisting of at least amino acids 50 to 110 of native human LAIR-1 having the mutations as described herein and having at least 70%, preferably at least 75%, more preferably at least 80%, even more preferably at least 85% and particularly preferably at least 87% sequence identity in comparison to amino acids 50 to 110 of native human LAIR-1 (SEQ ID NO: 11). Thereby, it is preferred if the protein according to the present invention comprises (or consists of) a LAIR-1 fragment comprising or consisting of an amino acid sequence according to SEQ ID NO: 15.

SEQ ID NO: 15

RGPXGXXTFRLXXXXXXX₁YX₂XXEXVXXX₃XPXXSEARFRXXSVXXGXX
GXXRCXYYXX₄X₅XWS

wherein
X is any amino acid or no amino acid (deletion mutation);
X₁ is any amino acid or no amino acid; however, if X₂ is N, X₃ is A, X₄ is P and X₅ is P, then X₁ is any amino acid except T or no amino acid;
X₂ is any amino acid; however, if X₁ is T, X₃ is A, X₄ is P and X₅ is P, then X₂ is any amino acid except N;
X₃ is any amino acid; however, if X₁ is T, X₂ is N, X₄ is P and X₅ is P, then X₃ is any amino acid except A;
X₄ is any amino acid; however, if X₁ is T, X₂ is N, X₃ is A and X₅ is P, then X₄ is any amino acid except P; and
X₅ is any amino acid; however, if X₁ is T, X₂ is N, X₃ is A and X₄ is P, then X₅ is any amino acid except P.

If an amino acid is substituted in a position “X” of SEQ ID NO: 15, such a substitution is preferably a conservative substitution as described herein.

More preferably, the protein according to the present invention comprises (or consists of) a LAIR-1 fragment consisting of at least amino acids 40 to 115 of native human LAIR-1 having the mutations as described herein and having at least 70%, preferably at least 75%, more preferably at least 80%, even more preferably at least 85% and particularly preferably at least 87% sequence identity in comparison to amino acids 40 to 115 of native human LAIR-1 (SEQ ID NO: 12). Thereby, it is preferred if the protein according to the present invention comprises (or consists of) a LAIR-1 fragment comprising or consisting of an amino acid sequence according to SEQ ID NO: 16.

SEQ ID NO: 16

XLGSXXTXVCRGPXGXXTFRLXXXXXXX₁YX₂XXEXVXXX₃XPXXSEAR
FRXXSVXXGXXGXXRCXYYXX₄X₅XWSXXSXX

wherein
X is any amino acid or no amino acid (deletion mutation);
X₁ is any amino acid or no amino acid; however, if X₂ is N, X₃ is A, X₄ is P and X₅ is P, then X₁ is any amino acid except T or no amino acid;
X₂ is any amino acid; however, if X₁ is T, X₃ is A, X₄ is P and X₅ is P, then X₂ is any amino acid except N;
X₃ is any amino acid; however, if X₁ is T, X₂ is N, X₄ is P and X₅ is P, then X₃ is any amino acid except A;
X₄ is any amino acid; however, if X₁ is T, X₂ is N, X₃ is A and X₅ is P, then X₄ is any amino acid except P; and

Xₛ is any amino acid; however, if X₁ is T, X₂ is N, X₃ is A and X₄ is P, then X₅ is any amino acid except P.

If an amino acid is substituted in a position “X” of SEQ ID NO: 16, such a substitution is preferably a conservative substitution as described herein.

Even more preferably, the protein according to the present invention comprises (or consists of) a LAIR-1 fragment consisting of at least amino acids 30 to 120 of native human LAIR-1 having the mutations as described herein and having at least 70%, preferably at least 75%, more preferably at least 80%, even more preferably at least 85% and particularly preferably at least 87% sequence identity in comparison to amino acids 30 to 120 of native human LAIR-1 (SEQ ID NO: 13). Thereby, it is preferred if the protein according to the present invention comprises (or consists of) a LAIR-1 fragment comprising or consisting of an amino acid sequence according to SEQ ID NO: 17.

SEQ ID NO: 17

XXSXXXXXXXXLGSXXTXVCRGPXGXXTFRLXXXXXXX₁YX₂XXEXVXX
X₃XSEARFRXXSVXXGXXGXXRCXYYXX₄X₅XWSXXSXXXXXXV

wherein
X is any amino acid or no amino acid (deletion mutation);
X₁ is any amino acid or no amino acid; however, if X₂ is N, X₃ is A, X₄ is P and X₅ is P, then X₁ is any amino acid except T or no amino acid;
X₂ is any amino acid; however, if X₁ is T, X₃ is A, X₄ is P and X₅ is P, then X₂ is any amino acid except N;
X₃ is any amino acid; however, if X₁ is T, X₂ is N, X₄ is P and X₅ is P, then X₃ is any amino acid except A;
X₄ is any amino acid; however, if X₁ is T, X₂ is N, X₃ is A and X₅ is P, then X₄ is any amino acid except P; and
X₅ is any amino acid; however, if X₁ is T, X₂ is N, X₃ is A and X₄ is P, then X₅ is any amino acid except P.

If an amino acid is substituted in a position “X” of SEQ ID NO: 17, such a substitution is preferably a conservative substitution as described herein.

Particularly preferably, the protein according to the present invention comprises (or consists of) a LAIR-1 fragment consisting of at least amino acids 24 to 121 of native human LAIR-1 having the mutations as described herein and having at least 70%, preferably at least 75%, more preferably at least 80%, even more preferably at least 85% and particularly preferably at least 87% sequence identity in comparison to amino acids 24 to 121 of native human LAIR-1 (SEQ ID NO: 14). Thereby, it is preferred if the protein according to the present invention comprises (or consists of) a LAIR-1 fragment comprising or consisting of an amino acid sequence according to SEQ ID NO: 18.

SEQ ID NO: 18

XXLPRPXXSXXXXXXXXLGSXXTXVCRGPXGXXTFRLXXXXXXX₁YX₂X
XEXVXXX₃XPXXSEARFRXXSVXXGXXGXXRCXYYXX₄X₅XWSXXS
XXXXXXVK

wherein
X is any amino acid or no amino acid (deletion mutation);
X₁ is any amino acid or no amino acid; however, if X₂ is N, X₃ is A, X₄ is P and X₅ is P, then X₁ is any amino acid except T or no amino acid;
X₂ is any amino acid; however, if X₁ is T, X₃ is A, X₄ is P and X₅ is P, then X₂ is any amino acid except N;
X₃ is any amino acid; however, if X₁ is T, X₂ is N, X₄ is P and X₅ is P, then X₃ is any amino acid except A;
X4 is any amino acid; however, if X₁ is T, X₂ is N, X₃ is A and X₅ is P, then X₄ is any amino acid except P; and
X₅ is any amino acid; however, if X₁ is T, X₂ is N, X₃ is A and X₄ is P, then X₅ is any amino acid except P.

If an amino acid is substituted in a position “X” of SEQ ID NO: 18, such a substitution is preferably a conservative substitution as described herein.

In the present invention it is preferred that the LAIR-1 fragment comprised by the protein according to the present invention (i) includes at least a mutation at the position T67; or (ii) includes at least a mutation at the position N69; or (iii) includes at least a mutation at the position A77; or (iv) includes at least a mutation at the position P106; or (v) includes at least a mutation at the position P107. Preferably, the LAIR-1 fragment comprised by the protein according to the present invention includes at least a mutation at the position N69, more preferably the LAIR-1 fragment comprised by the protein according to the present invention includes at least a mutation at the position N69 selected from the group consisting of N69S and N69T, even more preferably the LAIR-1 fragment comprised by the protein according to the present invention includes at least the mutation N69S.

It is also preferred that the LAIR-1 fragment comprised by the protein according to the present invention includes a mutation at least two of the following five positions: T67, N69, A77, P106, and P107. Thereby, the LAIR-1 fragment comprised by the protein according to the present invention may preferably include (i) at least a mutation at the position T67 and at the position N69; or (ii) at least a mutation at the position T67 and at the position A77; or (iii) at least a mutation at the position T67 and at the position P106; or (iv) at least a mutation at the position T67 and at the position P107; or (v) at least a mutation at the position N69 and at the position A77; or (vi) at least a mutation at the position N69 and at the position P106; or (vii) at least a mutation at the position N69 and at the position P107; or (viii) at least a mutation at the position A77 and at the position P106; or (ix) at least a mutation at the position A77 and at the position P107; or (x) at least a mutation at the position P106 and at the position P107.

More preferably, the LAIR-1 fragment comprised by the protein according to the present invention includes (i) at least a mutation at the position T67 and at the position N69, (ii) at least a mutation at the position T67 and at the position A77, or (iii) at least a mutation at the position A77 and at the position N69; even more preferably the LAIR-1 fragment comprised by the protein according to the present invention includes (i) at least a mutation at the position T67 selected from the group consisting of T67G, T67I, T67L, T67R, and T67K and at the position N69 selected from the group consisting of N69S and N69T, (ii) at least a mutation at the position T67 selected from the group consisting of T67G, T67I, T67L, T67R, and T67K and at the position A77 selected from the group consisting of A77T, A77P and A77V, or (iii) at least a mutation at the position A77 selected from the group consisting of A77T, A77P and A77V and at the position N69 selected from the group consisting of N69S and N69T; and particularly preferably the LAIR-1 fragment comprised by the protein according to the present invention includes (i) at least the mutations T67L and N69S, (ii) at least the mutations T67L and A77T, or (iii) at least the mutations N69S and A77T.

Preferably, the LAIR-1 fragment comprised by the protein according to the present invention includes a mutation at least three of the following five positions: T67, N69, A77, P106, and P107. Thereby, the LAIR-1 fragment comprised by the protein according to the present invention may preferably include (i) at least a mutation at the position T67, at the position N69 and at the position A77; or (ii) at least a mutation at the position T67, at the position N69 and at the position P106; or (iii) at least a mutation at the position T67, at the position N69 and at the position P107; or (iv) at least a mutation at the position T67, at the position A77 and at the position P106; or (v) at least a mutation at the position T67, at the position A77 and at the position P107; or (vi) at least a mutation at the position T67, at the position P106 and at the position P107; or (vii) at least a mutation at the position N69, at the position A77 and at the position P106; or (viii) at least a mutation at the position N69, at the position A77 and at the position P107; or (ix) at least a mutation at the position N69, at the position P106 and at the position P107; or (x) at least a mutation at the position A77, at the position P106 and at the position P107.

More preferably, the LAIR-1 fragment comprised by the protein according to the present invention includes (i) at least a mutation at the position T67, at the position N69 and at the position A77, (ii) at least a mutation at the position T67, at the position N69 and at the position P107 or (iii) at least a mutation at the position T67, at the position A77 and at the position P107; even more preferably the LAIR-1 fragment comprised by the protein according to the present invention includes (i) at least a mutation at the position T67 selected from the group consisting of T67G, T67I, T67L, T67R, and T67K, at the position N69 selected from the group consisting of N69S and N69T and at the position A77 selected from the group consisting of A77T, A77P and A77V, (ii) at least a mutation at the position T67 selected from the group consisting of T67G, T67I, T67L, T67R, and T67K, at the position N69 selected from the group consisting of N69S and N69T and at the position P107 selected from the group consisting of P107S and P107R or (iii) at least a mutation at the position T67 selected from the group consisting of T67G, T67I, T67L, T67R, and T67K, at the position A77 selected from the group consisting of A77T, A77P and A77V and at the position P107 selected from the group consisting of P107S and P107R; and particularly preferably the LAIR-1 fragment comprised by the protein according to the present invention includes (i) at least the mutations T67L, N69S and A77T, (ii) at least the mutations T67L, N69S and P107R, or (iii) at least the mutations T67L, A77T and P107R.

It is also preferred that the LAIR-1 fragment comprised by the protein according to the present invention includes a mutation at at least four of the following five positions: T67, N69, A77, P106, and P107. Thereby, the LAIR-1 fragment comprised by the protein according to the present invention may preferably include (i) at least a mutation at the position T67, at the position N69, at the position A77 and at the position P106; or (ii) at least a mutation at the position T67, at the position N69, at the position A77 and at the position P107; or (iii) at least a mutation at the position T67, at the position N69, at the position P106 and at the position P107; or (iv) at least a mutation at the position T67, at the position A77, at the position P106 and at the position P107; or (v) at least a mutation at the position N69, at the position A77, at the position P106 and at the position P107.

More preferably, the LAIR-1 fragment comprised by the protein according to the present invention includes (i) at least a mutation at the position T67, at the position N69, at the position A77, and at position P107 or (ii) at least a mutation at the position T67, at the position N69, at the position P106, and at position P107; even more preferably the LAIR-1 fragment comprised by the protein according to the present invention includes (i) at least a mutation at the position T67 selected from the group consisting of T67G, T67I, T67L, T67R, and T67K, at the position N69 selected from the group consisting of N69S and N69T, at the position A77 selected from the group consisting of A77T, A77P and A77V, and at the position P107 selected from the group consisting of P107S and P107R or (ii) at least a mutation at the position T67 selected from the group consisting of T67G, T67I, T67L, T67R, and T67K, at the position N69 selected from the group consisting of N69S and N69T, at the position P106 selected from the group consisting of P106S, P106A, and P106D, and at the position P107 selected from the group consisting of P107S and P107R; and particularly preferably the LAIR-1 fragment comprised by the protein according to the present invention includes (i) at least the mutations T67L, N69S, A77T and P107R or (ii) at least the mutations T67L, N69S, P106S and P107R.

Preferably, the LAIR-1 fragment comprised by the protein according to the present invention includes a mutation at each of the following five positions: T67, N69, A77, P106, and P107; more preferably the LAIR-1 fragment comprised by the protein according to the present invention includes a mutation at the position T67 selected from the group consisting of T67G, T67I, T67L, T67R, and T67K, at the position N69 selected from the group consisting of N69S and N691, at the position A77 selected from the group consisting of A77T, A77P and A77V, at the position P106 selected from the group consisting of P106S, P106A, and P106D and at the position P107 selected from the group consisting of P107S and P107R; and particularly preferably the LAIR-1 fragment comprised by the protein according to the present invention includes the mutations T67L, N69S, A77T, P106S and P107R.

In the present invention, it is preferred that the mutation is a deletion or a substitution, preferably the mutation is a substitution as described above.

If, in the protein according to the present invention, the threonine residue at position T67 (of native human LAIR-1) is mutated, the mutation at position T67 is preferably a deletion of the threonine residue or a substitution of the threonine residue by another single amino acid.

If, in the protein according to the present invention, the asparagine residue at position N69 (of native human LAIR-1) is mutated, the mutation at position N69 is preferably a substitution of the asparagine residue by another single amino acid.

If, in the protein according to the present invention, the alanine residue at position A77 (of native human LAIR-1) is mutated, the mutation at position A77 is preferably a substitution of the alanine residue by another single amino acid.

If, in the protein according to the present invention, the proline residue at position P106 (of native human LAIR-1) is mutated, the mutation at position P106 is preferably a substitution of the proline residue by another single amino acid.

If, in the protein according to the present invention, the proline residue at position P107 (of native human LAIR-1) is mutated, the mutation at position P107 is preferably a substitution of the proline residue by another single amino acid.

More preferably, in the protein according to the present invention the mutation at position T67 is a deletion of the threonine residue or a substitution of the threonine residue by another single amino acid; the mutation at position N69 is a substitution of the asparagine residue by another single amino acid; the mutation at position A77 is a substitution of the alanine residue by another single amino acid; the mutation at position P106 is a substitution of the proline residue by another single amino acid; and the mutation at position P107 is a substitution of the proline residue by another single amino acid.

If, in the protein according to the present invention, the threonine residue at position T67 (of native human LAIR-1) is mutated, the threonine residue at position T67 is preferably either (i) deleted or (ii) substituted by an amino acid. If the threonine residue at position T67 is substituted by an amino acid, it is preferably substituted by an amino acid which is either (a) aliphatic and nonpolar or (b) positively charged.

An “aliphatic” amino acid, as used herein, refers to any amino acid selected from the group consisting of alanine, glycine, isoleucine, leucine, and valine. A “nonpolar” amino acid, as used herein, refers to any amino acid selected from the group consisting of alanine, cysteine, phenylalanine, glycine, isoleucine, leucine, methionine, proline and valine. A “positively charged” amino acid, as used herein, refers to any amino acid selected from the group consisting of arginine, histidine and lysine.

Accordingly, if, in the LAIR-1 fragment according to the present invention, the threonine residue at position T67 (of native human LAIR-1) is substituted, a substitution is preferably selected from the group consisting of T67A, T67G, T67I, T67L, T67V, T67R, T67H, and T67K.

More preferably, the threonine residue at position T67 (of native human LAIR-1) is substituted in the LAIR-1 fragment according to the present invention by an amino acid selected from the group consisting of leucine, glycine, isoleucine, arginine and lysine. Thus, a substitution is preferably selected from the group consisting of T67G, T67I, T67L, T67R, and T67K.

Even more preferably, the threonine residue at position T67 (of native human LAIR-1) is substituted in the LAIR-1 fragment according to the present invention by leucine (T67L).

If, in the protein according to the present invention, the asparagine residue at position N69 (of native human LAIR-1) is mutated, the asparagine residue at position N69 is preferably substituted, more preferably the asparagine residue at position N69 is substituted by a small, polar amino acid.

A “small” amino acid, as used herein, refers to any amino acid selected from the group consisting of alanine, aspartic acid, asparagine, cysteine, glycine, proline, serine, threonine and valine. A “polar” amino acid, as used herein, refers to any amino acid selected from the group consisting of aspartic acid, asparagine, arginine, glutamic acid, histidine, lysine, glutamine, tryptophan, tyrosine, serine, and threonine.

Accordingly, if, in the LAIR-1 fragment according to the present invention, the asparagine residue at position N69 (of native human LAIR-1) is substituted, a substitution is preferably selected from the group consisting of N69D, N69S and N69T.

More preferably, the asparagine residue at position N69 (of native human LAIR-1) is substituted in the LAIR-1 fragment according to the present invention by an amino acid selected from the group consisting of serine and threonine. Thus, a substitution is preferably selected from the group consisting of N69S and N69T.

Even more preferably, the asparagine residue at position N69 (of native human LAIR-1) is substituted in the LAIR-1 fragment according to the present invention by serine (N69S).

If, in the protein according to the present invention, the alanine residue at position A77 (of native human LAIR-1) is mutated, the alanine residue at position A77 is preferably substituted, more preferably the alanine residue at position A77 is substituted by a small amino acid.

Accordingly, if, in the LAIR-1 fragment according to the present invention, the alanine residue at position A77 (of native human LAIR-1) is substituted, a substitution is preferably selected from the group consisting of A77D, A77N, A77C, A77G, A77P, A77S, A77T, and A77V.

More preferably, the alanine residue at position A77 (of native human LAIR-1) is substituted in the LAIR-1 fragment according to the present invention by an amino acid selected from the group consisting threonine, proline and valine. Thus, a substitution is preferably selected from the group consisting of A77T, A77P and A77V.

Even more preferably, the alanine residue at position A77 (of native human LAIR-1) is substituted in the LAIR-1 fragment according to the present invention by threonine (A77T).

If, in the protein according to the present invention, the proline residue at position P106 (of native human LAIR-1) is mutated, the proline residue at position P106 is preferably substituted, more preferably the proline residue at position P106 is substituted by a small amino acid.

Accordingly, if, in the LAIR-1 fragment according to the present invention, the proline residue at position P106 (of native human LAIR-1) is substituted, a substitution is preferably selected from the group consisting of P106A, P106D, P106N, P106C, P106G, P106S, P106T, and P106V.

More preferably, the proline residue at position P106 (of native human LAIR-1) is substituted in the LAIR-1 fragment according to the present invention by an amino acid selected from the group consisting of serine, alanine and aspartic acid. Thus, a substitution is preferably selected from the group consisting of P106S, P106A, and P106D.

Even more preferably, the proline residue at position P106 (of native human LAIR-1) is substituted in the LAIR-1 fragment according to the present invention by serine (P106S).

If, in the protein according to the present invention, the proline residue at position P107 (of native human LAIR-1) is mutated, the the proline residue at position P107 is preferably substituted, more preferably the proline residue at position P107 is substituted by a polar amino acid, whereby in particular a positively charged amino acid may be preferred.

Accordingly, if, in the LAIR-1 fragment according to the present invention, the proline residue at position P107 (of native human LAIR-1) is substituted, a substitution is preferably selected from the group consisting of P107S, P107T, P107N, P107Q, P107Y, P107W, P107E, P107D, P107R, P107K, and P107H, in particular preferably selected from the group consisting of P107R, P107K, and P107H.

More preferably, the proline residue at position P107 (of native human LAIR-1) is substituted in the LAIR-1 fragment according to the present invention by an amino acid selected from the group consisting of serine and arginine. Thus, a substitution is preferably selected from the group consisting of P107S and P107R.

Even more preferably, the proline residue at position P107 (of native human LAIR-1) is substituted in the LAIR-1 fragment according to the present invention by arginine (P107R).

Thus, it is preferred in the present invention, if the LAIR-1 fragment comprised by the protein according to the present invention has an amino acid sequence according to SEQ ID NO: 19 as shown below, more preferably according to SEQ ID NO: 20, and—as described above—has at least 70% amino acid sequence identity to amino acids 67 to 107 of native human LAIR-1 (SEQ ID NO: 9).

SEQ ID NO: 19

X₁YX₂XXEXVXXX₃XPXXSEARFRXXSVXXGXXGXXRCXYYXX₄X₅

wherein
X is any amino acid;
X₁ is T, L, G, I, R, K or no amino acid; however, if X₂ is N, X₃ is A, X₄ is P and X₅ is P, then X₁ is L, G, I, R, K or no amino acid;
X₂ is N, S or T; however, if X₁ is T, X₃ is A, X₄ is P and X₅ is P, then X₂ is S or T;
X₃ is A, T, P, or V; however, if X₁ is T, X₂ is N, X₄ is P and X₅ is P, then X₃ is T, P, or V;
X₄ is P, S, A, or D; however, if X₁ is T, X₂ is N, X₃ is A and X₅ is P, then X₄ is S, A, or D; and
X₅ is P, R, or S; however, if X₁ is T, X₂ is N, X₃ is A and X₄ is P, then X₅ is R, or S.

SEQ ID NO: 20

X₁YX₂XXEXVXXX₃XPXXSEARFRXXSVXXGXXGXXRCXYYXX₄X₅

wherein
X is any amino acid;
X₁ is T or L; however, if X₂ is N, X₃ is A, X₄ is P and X₅ is P, then X₁ is L;
X₂ is N or S; however, if X₁ is T, X₃ is A, X₄ is P and X₅ is P, then X₂ is S;
X₃ is A or T; however, if X₁ is T, X₂ is N, X4 is P and X₅ is P, then X₃ is T;
X₄ is P or S; however, if X₁ is T, X₂ is N, X₃ is A and X₅ is P, then X₄ is S; and
X₅ is P or R; however, if X₁ is T, X₂ is N, X₃ is A and X₄ is P, then X₅ is R.

If an amino acid is substituted in a position “X” of SEQ ID NO: 19 or 20, such a substitution is preferably a conservative substitution as described herein.

It is even more preferred in the present invention, if the LAIR-1 fragment comprised by the protein according to the present invention has an amino acid sequence according to SEQ ID NO: 21 as shown below, more preferably according to SEQ ID NO: 22, and—as described above—has at least 70% amino acid sequence identity to amino acids 24 to 121 of native human LAIR-1 (SEQ ID NO: 14).

SEQ ID NO: 21

XXLPRPXXSXXXXXXXXLGSXXTXVCRGPXGXXTFRLXXXXXXX₁YX₂X
XEXVXXX₃XPXXSEARFRXXSVXXGXXGXXRCXYYXX₄X₅XWSXXSX
XXXXXVK

wherein
X is any amino acid or no amino acid;
X₁ is T, L, G, I, R, K or no amino acid; however, if X₂ is N, X₃ is A, X₄ is P and X₅ is P, then X₁ is L, G, I, R, K or no amino acid;
X₂ is N, S or T; however, if X₁ is T, X₃ is A, X₄ is P and X₅ is P, then X₂ is S or T;
X₃ is A, T, P, or V; however, if X₁ is T, X₂ is N, X₄ is P and X₅ is P, then X₃ is T, P, or V;
X₄ is P, S, A, or D; however, if X₁ is T, X₂ is N, X₃ is A and X₅ is P, then X₄ is S, A, or D; and
X₅ is P, R, or S; however, if X₁ is T, X₂ is N, X₃ is A and X₄ is P, then X₅ is R or S.

Preferably, X is any amino acid (substitution mutation). If an amino acid is substituted in a position “X” of SEQ ID NO: 21, such a substitution is preferably a conservative substitution as described herein.

SEQ ID NO: 22

XXLPRPXXSXXXXXXXXLGSXXTXVCRGPXGXXTFRLXXXXXXX₁YX₂X
XEXVXXX₃XPXXSEARFRXXSVXXGXXGXXRCXYYXX₄X₅XWSXXSX
XXXXXVK

wherein
X is any amino acid or no amino acid;
X₁ is T or L; however, if X₂ is N, X₃ is A, X₄ is P and X₅ is P, then X₁ is L;
X₂ is N or S; however, if X₁ is T, X₃ is A, X₄ is P and X₅ is P, then X₂ is S;
X₃ is A or T; however, if X₁ is T, X₂ is N, X₄ is P and X₅ is P, then X₃ is T;
X₄ is P or S; however, if X₁ is T, X₂ is N, X₃ is A and X₅ is P, then X₄ is S; and
X₅ is P or R; however, if X₁ is T, X₂ is N, X₃ is A and X₄ is P, then X₅ is R.

Preferably, X is any amino acid (substitution mutation). If an amino acid is substituted in a position “X” of SEQ ID NO: 22, such a substitution is preferably a conservative substitution as described herein.

Amino acid sequences (and exemplary nucleic acid sequences encoding these amino acid sequences) of preferred examples of LAIR-1 fragments comprised by a protein according to the present invention are shown below in Table 1.

TABLE 1
Sequences and Seq ID NOs of Amino acid sequences (and exemplary nucleic acid
sequences encoding these amino acid sequences) of preferred examples of
LAIR-1 fragments

| SEQ | | |
|---|---|---|
| ID | | |
| NO | Description | Sequence* |
| 23 | LAIR1ex + L aa | EDLPRPSISAEPGTVIPLGSHVTFVCRGPVGVQTFRLERESRSLY |
| | | NDTEDVSQASPSESEARFRIDSVSEGNAGPYRCIYYKPPKWSE |
| | | QSDYLELLVK |
| 24 | LAIR1ex + L nucl | GAGGACCTGCCAAGACCCAGCATCTCCGCAGAACCTGG |
| | | GACCGTGATTCCACTGGGCTCCCACGTGACATTCGTCTGC |
| | | AGAGGCCCCGTGGGAGTCCAGACTTTTAGGCTGGAGCG |
| | | CGAATCTCGAAGTCTGTACAACGACACAGAGGACGTGAG |
| | | CCAGGCCTCACCAAGCGAGTCCGAAGCTCGGTTCAGAAT |
| | | CGACTCTGTCAGTGAAGGAAATGCCGGCCCTTACAGATG |
| | | CATCTACTATAAGCCCCCTAAATGGTCAGAGCAGAGCGAT |
| | | TATCTGGAACTGCTGGTGAAG |
| 25 | LAIR1ex + LR aa | EDLPRPSISAEPGTVIPLGSHVTFVCRGPVGVQTFRLERESRSLY |
| | | NDTEDVSQASPSESEARFRIDSVSEGNAGPYRCIYYKPRKWSE |
| | | QSDYLELLVK |
| 26 | LAIR1ex + LR nucl | GAGGACCTGCCCCGCCCTAGCATCTCCGCAGAACCAGG |
| | | GACCGTGATTCCCCTGGGCTCCCACGTGACATTCGTCTGC |
| | | AGGGGCCCCGTGGGAGTCCAGACTTTTAGGCTGGAGCG |
| | | CGAATCTCGAAGTCTGTACAACGACACCGAGGACGTGAG |
| | | CCAGGCCTCACCTAGCGAGTCCGAAGCTCGGTTCAGAAT |
| | | CGACTCTGTCAGTGAAGGAAATGCCGGCCCTTACAGATG |
| | | CATCTACTATAAGCCAAGAAAATGGTCAGAGCAGAGCGA |
| | | TTATCTGGAACTGCTGGTGAAG |
| 27 | LAIR1ex + LS1 aa | EDLPRPSISAEPGTVIPLGSHVTFVCRGPVGVQTFRLERESRSLY |
| | | SDTEDVSQASPSESEARFRIDSVSEGNAGPYRCIYYKPPKWSEQ |
| | | SDYLELLVK |
| 28 | LAIR1ex + LS1 | GAGGACCTGCCAAGACCCAGCATCTCCGCAGAACCTGG |
| | nucl | GACCGTGATTCCACTGGGCTCCCACGTGACATTCGTCTGC |
| | | AGAGGCCCCGTGGGAGTCCAGACTTTTAGGCTGGAGCG |
| | | CGAATCTCGAAGTCTGTACTCCGACACAGAGGACGTGAG |
| | | CCAGGCCTCACCAAGCGAGTCCGAAGCTCGGTTCAGAAT |
| | | CGACTCTGTCAGTGAAGGAAACGCCGGCCCTTACAGATG |
| | | CATCTACTATAAGCCCCCTAAATGGTCAGAGCAGAGCGAT |
| | | TATCTGGAACTGCTGGTGAAG |
| 29 | LAIR1ex + LS1R | EDLPRPSISAEPGTVIPLGSHVTFVCRGPVGVQTFRLERESRSLY |
| | aa | SDTEDVSQASPSESEARFRIDSVSEGNAGPYRCIYYKPRKWSEQ |
| | | SDYLELLVK |
| 30 | LAIR1ex + LS1R | GAGGACCTGCCCCGCCCTAGCATCTCCGCAGAACCAGG |
| | nucl | GACCGTGATTCCCCTGGGCTCCCACGTGACATTCGTCTGC |
| | | AGGGGCCCCGTGGGAGTCCAGACTTTTAGGCTGGAGCG |
| | | CGAATCTCGAAGTCTGTACTCCGACACCGAGGACGTGAG |
| | | CCAGGCCTCACCTAGCGAGTCCGAAGCTCGGTTCAGAAT |
| | | CGACTCTGTCAGTGAAGGAAACGCCGGCCCTTACAGATG |
| | | CATCTACTATAAGCCAAGAAAATGGTCAGAGCAGAGCGA |
| | | TTATCTGGAACTGCTGGTGAAG |
| 31 | LAIR1ex + LS1S2R | EDLPRPSISAEPGTVIPLGSHVTFVCRGPVGVQTFRLERESRSLY |
| | aa | SDTEDVSQASPSESEARFRIDSVSEGNAGPYRCIYYKSRKWSEQ |
| | | SDYLELLVK |
| 32 | LAIR1ex + LS1S2R | GAGGACCTGCCCCGCCCTAGCATCTCCGCAGAACCAGG |
| | nucl | GACCGTGATTCCCCTGGGCTCCCACGTGACATTCGTCTGC |
| | | AGGGGCCCCGTGGGAGTCCAGACTTTTAGGCTGGAGCG |
| | | CGAATCTCGAAGTCTGTACTCCGACACCGAGGACGTGAG |
| | | CCAGGCCTCACCTAGCGAGTCCGAAGCTCGGTTCAGAAT |
| | | CGACTCTGTCAGTGAAGGAAACGCCGGCCCATACAGATG |
| | | CATCTACTATAAGAGCAGAAAATGGTCAGAGCAGAGCGA |
| | | TTATCTGGAACTGCTGGTGAAG |
| 33 | LAIR1ex + LS1T aa | EDLPRPSISAEPGTVIPLGSHVTFVCRGPVGVQTFRLERESRSLY |
| | | SDTEDVSQTSPSESEARFRIDSVSEGNAGPYRCIYYKPPKWSEQ |
| | | SDYLELLVK |
| 34 | LAIR1ex + LS1T | GAGGACCTGCCAAGACCCAGCATCTCCGCCGAACCTGG |
| | nucl | GACTGTGATTCCACTGGGCTCCCACGTGACCTTCGTCTGC |
| | | AGAGGCCCCGTGGGAGTCCAGACCTTCCGGCTGGAGCG |
| | | CGAATCTCGAAGTCTGTACTCCGACACCGAGGACGTGAG |
| | | CCAGACATCACCCAGCGAGTCCGAAGCCCGGTTCAGAAT |
| | | CGACTCTGTCAGTGAAGGAAACGCTGGCCCTTACAGATG |
| | | CATCTACTATAAGCCCCCTAAATGGTCAGAGCAGAGCGAT |
| | | TATCTGGAACTGCTGGTGAAG |
| 35 | LAIR1ex + LS1TR | EDLPRPSISAEPGTVIPLGSHVTFVCRGPVGVQTERLERESRSLY |
| | aa | SDTEDVSQTSPSESEARFRIDSVSEGNAGPYRCIYYKPRKWSEQ |
| | | SDYLELLVK |
| 36 | LAIR1ex + LS1TR | GAGGACCTGCCTAGACCTAGCATCTCCGCCGAACCAGGG |
| | nucl | ACTGTGATTCCCCTGGGCTCCCACGTGACCTTCGTCTGCA |
| | | GAGGCCCCGTGGGAGTCCAGACCTTCCGGCTGGAGCGC |
| | | GAATCTCGAAGTCTGTACTCCGACACCGAGGACGTGAGC |
| | | CAGACATCACCTAGCGAGTCCGAAGCCCGGTTCAGAATC |
| | | GACTCTGTCAGTGAAGGAAACGCTGGCCCTTACAGATGC |
| | | ATCTACTATAAGCCAAGAAAATGGTCAGAGCAGAGCGAT |
| | | TATCTGGAACTGCTGGTGAAG |
| 37 | LAIR1ex + LS1TS2R | EDLPRPSISAEPGTVIPLGSHVTFVCRGPVGVQTFRLERESRSLY |
| | aa | SDTEDVSQTSPSESEARFRIDSVSEGNAGPYRCIYYKSRKWSEQ |
| | | SDYLELLVK |
| 38 | LAIR1ex + LS1TS2R | GAGGACCTGCCTAGACCTAGCATCTCCGCCGAACCAGGG |
| | nucl | ACTGTGATTCCCCTGGGCTCCCACGTGACCTTCGTCTGCA |
| | | GAGGCCCCGTGGGAGTCCAGACCTTCCGGCTGGAGCGC |
| | | GAATCTCGAAGTCTGTACTCCGACACCGAGGACGTGAGC |
| | | CAGACATCACCTAGCGAGTCCGAAGCCCGGTTCAGAATC |
| | | GACTCTGTCAGTGAAGGAAACGCTGGCCCATACAGATGC |
| | | ATCTACTATAAGAGCAGAAAATGGTCAGAGCAGAGCGAT |
| | | TATCTGGAACTGCTGGTGAAG |
| 39 | LAIR1ex + LS2R | EDLPRPSISAEPGTVIPLGSHVTFVCRGPVGVQTFRLERESRSLY |
| | aa | NDTEDVSQASPSESEARFRIDSVSEGNAGPYRCIYYKSRKWSE |
| | | QSDYLELLVK |
| 40 | LAIR1ex + LS2R | GAGGACCTGCCCCGCCCTAGCATCTCCGCAGAACCAGG |
| | nucl | GACCGTGATTCCCCTGGGCTCCCACGTGACATTCGTCTGC |
| | | AGGGGCCCCGTGGGAGTCCAGACTTTTAGGCTGGAGCG |
| | | CGAATCTCGAAGTCTGTACAACGACACCGAGGACGTGAG |
| | | CCAGGCCTCACCTAGCGAGTCCGAAGCTCGGTTCAGAAT |
| | | CGACTCTGTCAGTGAAGGAAATGCCGGCCCATACAGATG |
| | | CATCTACTATAAGTCTAGAAAATGGTCAGAGCAGAGCGAT |
| | | TATCTGGAACTGCTGGTGAAG |
| 41 | LAIR1ex + LT aa | EDLPRPSISAEPGTVIPLGSHVTFVCRGPVGVQTFRLERESRSLY |
| | | NDTEDVSQTSPSESEARFRIDSVSEGNAGPYRCIYYKPPKWSE |
| | | QSDYLELLVK |
| 42 | LAIR1ex + LT nucl | GAGGACCTGCCAAGACCCAGCATCTCCGCCGAACCTGG |
| | | GACTGTGATTCCACTGGGCTCCCACGTGACCTTCGTCTGC |
| | | AGAGGCCCCGTGGGAGTCCAGACCTTCCGGCTGGAGCG |
| | | CGAATCTCGAAGTCTGTACAACGACACCGAGGACGTGAG |
| | | CCAGACATCACCCAGCGAGTCCGAAGCCCGGTTCAGAAT |
| | | CGACTCTGTCAGTGAAGGAAATGCTGGCCCTTACAGATG |
| | | CATCTACTATAAGCCCCCTAAATGGTCAGAGCAGAGCGAT |
| | | TATCTGGAACTGCTGGTGAAG |
| 43 | LAIR1ex + R aa | EDLPRPSISAEPGTVIPLGSHVTFVCRGPVGVQTERLERESRSTY |
| | | NDTEDVSQASPSESEARFRIDSVSEGNAGPYRCIYYKPRKWSE |
| | | QSDYLELLVK |
| 44 | LAIR1ex + R nucl | GAGGACCTGCCCCGCCCTAGCATCTCCGCAGAACCAGG |
| | | GACTGTGATTCCCCTGGGCTCCCACGTGACCTTCGTCTGC |
| | | AGAGGCCCCGTGGGAGTCCAGACCTTCCGGCTGGAGCG |
| | | CGAATCTCGAAGTACCTACAACGACACAGAGGACGTGAG |
| | | CCAGGCCTCACCTAGCGAGTCCGAAGCTCGGTTCAGAAT |
| | | CGACTCTGTCAGTGAAGGAAATGCCGGCCCTTACAGATG |
| | | CATCTACTATAAGCCAAGAAAATGGTCAGAGCAGAGCGA |
| | | TTATCTGGAACTGCTGGTGAAG |
| 45 | LAIR1ex + S1 aa | EDLPRPSISAEPGTVIPLGSHVTFVCRGPVGVQTFRLERESRSTY |
| | | SDTEDVSQASPSESEARFRIDSVSEGNAGPYRCIYYKPPKWSEQ |
| | | SDYLELLVK |
| 46 | LAIR1ex + S1 nucl | GAGGACCTGCCAAGACCCAGCATCTCCGCAGAACCTGG |
| | | GACTGTGATTCCACTGGGCTCCCACGTGACCTTCGTCTGC |
| | | AGAGGCCCCGTGGGAGTCCAGACCTTCCGGCTGGAGCG |
| | | CGAATCTCGAAGTACCTACTCCGACACAGAGGACGTGAG |
| | | CCAGGCCTCACCCAGCGAGTCCGAAGCTCGGTTCAGAAT |
| | | CGACTCTGTCAGTGAAGGAAACGCCGGCCCTTACAGATG |
| | | CATCTACTATAAGCCCCCTAAATGGTCAGAGCAGAGCGAT |
| | | TATCTGGAACTGCTGGTGAAG |
| 47 | LAIR1ex + S1R aa | EDLPRPSISAEPGTVIPLGSHVTFVCRGPVGVQTERLERESRSTY |
| | | SDTEDVSQASPSESEARFRIDSVSEGNAGPYRCIYYKPRKWSEQ |
| | | SDYLELLVK |
| 48 | LAIR1ex + S1R | GAGGACCTGCCCCGCCCTAGCATCTCCGCAGAACCAGG |
| | nucl | GACTGTGATTCCCCTGGGCTCCCACGTGACCTTCGTCTGC |
| | | AGAGGCCCCGTGGGAGTCCAGACCTTCCGGCTGGAGCG |
| | | CGAATCTCGAAGTACCTACTCCGACACAGAGGACGTGAG |
| | | CCAGGCCTCACCTAGCGAGTCCGAAGCTCGGTTCAGAAT |
| | | CGACTCTGTCAGTGAAGGAAACGCCGGCCCTTACAGATG |
| | | CATCTACTATAAGCCAAGAAAATGGTCAGAGCAGAGCGA |
| | | TTATCTGGAACTGCTGGTGAAG |
| 49 | LAIR1ex + S1S2R | EDLPRPSISAEPGTVIPLGSHVTFVCRGPVGVQTERLERESRSTY |
| | aa | SDTEDVSQASPSESEARFRIDSVSEGNAGPYRCIYYKSRKWSEQ |
| | | SDYLELLVK |
| 50 | LAIR1ex + S1S2R | GAGGACCTGCCCCGCCCTAGCATCTCCGCAGAACCAGG |
| | nucl | GACTGTGATTCCCUGGGCTCCCACGTGACCTICGTCTGC |
| | | AGAGGCCCCGTGGGAGTCCAGACCTTCCGGCTGGAGCG |
| | | CGAATCTCGAAGTACCTACTCCGACACAGAGGACGTGAG |
| | | CCAGGCCTCACCTAGCGAGTCCGAAGCTCGGTTCAGAAT |
| | | CGACTCTGTCAGTGAAGGAAACGCCGGCCCATACAGATG |
| | | CATCTACTATAAGAGCAGAAAATGGTCAGAGCAGAGCGA |
| | | TTATCTGGAACTGCTGGTGAAG |
| 51 | LAIR1ex + S1T aa | EDLPRPSISAEPGTVIPLGSHVTFVCRGPVGVQTFRLERESRSTY |
| | | SDTEDVSQTSPSESEARFRIDSVSEGNAGPYRCIYYKPPKWSEQ |
| | | SDYLELLVK |
| 52 | LAIR1ex + S1T | GAGGACCTGCCAAGACCCAGCATCTCCGCCGAACCTGG |
| | nucl | GACTGTGATTCCACTGGGCTCCCACGTGACCTTCGTCTGC |
| | | AGAGGCCCCGTGGGAGTCCAGACCTTCCGGCTGGAGCG |
| | | CGAATCTCGAAGTACCTACTCCGACACAGAGGACGTGAG |
| | | CCAGACCTCACCCAGCGAGTCCGAAGCCCGGTTCAGAAT |
| | | CGACTCTGTCAGTGAAGGAAACGCTGGCCCTTACAGATG |
| | | CATCTACTATAAGCCCCCTAAATGGTCAGAGCAGAGCGAT |
| | | TATCTGGAACTGCTGGTGAAG |
| 53 | LAIR1ex + S2 aa | EDLPRPSISAEPGTVIPLGSHVTFVCRGPVGVQTFRLERESRSTY |
| | | NDTEDVSQASPSESEARFRIDSVSEGNAGPYRCIYYKSPKWSE |
| | | QSDYLELLVK |
| 54 | LAIR1ex + S2 nucl | GAGGACCTGCCCAGACCTAGCATCTCCGCAGAACCAGG |
| | | GACTGTGATTCCCCTGGGCTCCCACGTGACCTTCGTCTGC |
| | | AGAGGCCCCGTGGGAGTCCAGACCTTCCGGCTGGAGCG |
| | | CGAATCTCGAAGTACCTACAACGACACAGAGGACGTGAG |
| | | CCAGGCCTCACCTAGCGAGTCCGAAGCTCGGTTCAGAAT |
| | | CGACTCTGTCAGTGAAGGAAATGCCGGCCCTTACAGATG |
| | | CATCTACTATAAGTCTCCAAAATGGTCAGAGCAGAGCGAT |
| | | TATCTGGAACTGCTGGTGAAG |
| 55 | LAIR1ex + S2R aa | EDLPRPSISAEPGTVIPLGSHVTFVCRGPVGVQTFRLERESRSTY |
| | | NDTEDVSQASPSESEARFRIDSVSEGNAGPYRCIYYKSRKWSE |
| | | QSDYLELLVK |
| 56 | LAIR1ex + S2R | GAGGACCTGCCCCGCCCTAGCATCTCCGCAGAACCAGG |
| | nucl | GACTGTGATTCCCCTGGGCTCCCACGTGACCTTCGTCTGC |
| | | AGAGGCCCCGTGGGAGTCCAGACCTTCCGGCTGGAGCG |
| | | CGAATCTCGAAGTACCTACAACGACACAGAGGACGTGAG |
| | | CCAGGCCTCACCTAGCGAGTCCGAAGCTCGGTTCAGAAT |
| | | CGACTCTGTCAGTGAAGGAAATGCCGGCCCATACAGATG |
| | | CATCTACTATAAGTCTAGAAAATGGTCAGAGCAGAGCGAT |
| | | TATCTGGAACTGCTGGTGAAG |
| 57 | LAIR1ex + T aa | EDLPRPSISAEPGTVIPLGSHVTFVCRGPVGVQTFRLERESRSTY |
| | | NDTEDVSQTSPSESEARFRIDSVSEGNAGPYRCIYYKPPKWSE |
| | | QSDYLELLVK |
| 58 | LAIR1ex + T nucl | GAGGACCTGCCAAGACCCAGCATCTCCGCCGAACCTGG |
| | | GACTGTGATTCCACTGGGCTCCCACGTGACCTTCGTCTGC |
| | | AGAGGCCCCGTGGGAGTCCAGACCTTCCGGCTGGAGCG |
| | | CGAATCTCGAAGTACCTACAACGACACAGAGGACGTGAG |
| | | CCAGACCTCACCCAGCGAGTCCGAAGCCCGGTTCAGAAT |
| | | CGACTCTGTCAGTGAAGGAAATGCTGGCCCTTACAGATG |
| | | CATCTACTATAAGCCCCCTAAATGGTCAGAGCAGAGCGAT |
| | | TATCTGGAACTGCTGGTGAAG |
| 59 | LAIR1ex + TS2R | EDLPRPSISAEPGTVIPLGSHVTFVCRGPVGVQTFRLERESRSTY |
| | aa | NDTEDVSQTSPSESEARFRIDSVSEGNAGPYRCIYYKSRKWSE |
| | | QSDYLELLVK |
| 60 | LAIR1ex + TS2R | GAGGACCTGCCTAGACCTAGCATCTCCGCCGAACCAGGG |
| | nucl | ACTGTGATTCCCCTGGGCTCCCACGTGACCTTCGTCTGCA |
| | | GAGGCCCCGTGGGAGTCCAGACCTTCCGGCTGGAGCGC |
| | | GAATCTCGAAGTACCTACAACGACACAGAGGACGTGAGC |
| | | CAGACCTCACCTAGCGAGTCCGAAGCCCGGTTCAGAATC |
| | | GACTCTGTCAGTGAAGGAAATGCTGGCCCATACAGATGC |
| | | ATCTACTATAAGTCTAGAAAATGGTCAGAGCAGAGCGATT |
| | | ATCTGGAACTGCTGGTGAAG |
| 61 | MGC1/MGC32_EXON | EDLPRPSISAAEGTVIPLGSHVTFVCRGPVGVQTFRLEKDSRSIY |
| | aa | NDTENVSQPSPSESEARFRIDSVSEGNAGLYRCVYYKAPKWSA |
| | | QSDYLELLVK |
| 62 | MGC1/MGC32_EXON | Gaagatctgcccagaccctccatctcggctgccgaaggcaccgtgatccccctggg |
| | nucl | gagccatgtgactacgtgtgccggggcccggttggggttcaaacattccgcctggaga |
| | | aggacagtagatccatatacaatgatactgaaaatgtgtctcaacctagtccatctgagt |
| | | cagaggccagatttcgcattgactcagtgagtgaaggaaatgccggactttatcggtgc |
| | | gtctattataaggcccctaaatggtctgcgcagagtgattacctggagctgctggtgaaa |
| 63 | MGC2_EXON aa | EHLPRPSISPEPGTVITLGSHVTFVCRGPVGVQTFRLEKDSRSTY |
| | | NDTEDVSQPSPSESEARFRIDSVSEGYAGLYRCLYYKPPKWSE |
| | | QSDYLELLVK |
| 64 | MGC2_EXON | gagcatctgcccagaccctccatctcgcctgagccaggcaccgtgatcaccctgggg |
| | nucl | agccatgtgactacgtgtgccggggcccggttggggttcaaacattccgcctggagaa |
| | | ggacagtagatccacatacaatgatactgaagatgtgtctcaacctagtccatctgagtc |
| | | agaggccagattccgcattgactcagtaagtgaaggatatgccgggctttatcgctgcct |
| | | ctattataagccccctaaatggtctgagcagagtgactacctggagctgctggtgaaa |
| 65 | MGC4_EXON aa | EDLPRPSISAEPDTVIPLGSHVTFVCRGPVGVHTFRLERGWRY |
| | | NDTEDVSQAGPSESEARFRIDSVREGNAGLYRCIYYIAPKWSE |
| | | QSDYLELRVK |
| 66 | MGC4_EXON | Gaagatctgcccagaccctccatctcggctgagccagacaccgtaatccccctggg |
| | nucl | gagccatgtgactttcgtgtgccggggcccggttggggttcacacattccgcctggaga |
| | | gggggtggaggtacaacgacactgaagatgtgtctcaagctggtccatctgagtcaga |
| | | ggccagattccgcattgactcggtaagggaaggaaatgccgggctttatcgatgcatct |
| | | attacatagcccctaaatggtctgagcagagtgactacctggagctgcgggtgaaa |
| 67 | MGC5/MGC29_EXON | EDLPRPSISAEPGTV1PLGSHVTFVCRGPVGVHTFRLERGWRY |
| | aa | NDTEDVSQAGPSQSEARFRIDSVREGNAGLYRCLYYIPPKWSE |
| | | QSDYLELRVK |
| 68 | MGC5/MGC29_EXON | Gaagatctgcccagaccctccatctcggctgagccaggcaccgtgatccccctggg |
| | nucl | gagccatgtgactttcgtgtgccggggccccgttggggttcacacattccgcctggaga |
| | | gggggtggagatacaacgacactgaagatgtgtctcaagctggtccatctcagtcaga |
| | | ggccagattccgcattgactcggtaagggaaggaaatgccgggctttatcgatgcctct |
| | | attacataccccctaaatggtctgagcagagtgactacctggaactgcgggtgaaa |
| 69 | MGC7/MGC37_EXON | DDLPRPSISPEPGTVIPLGSHVTFVCRGPVGVQTFRLEKDRRST |
| | aa | YNDTEDVSQPSPSESEARFRIDSVTEGNAGLYRCVYYKPPKWS |
| | | DQSDFLELLVK |
| 70 | MGC7/MGC37_EXON | Gatgatctgcccagaccctctatctcgcctgagccaggcaccgtgatccccctgggg |
| | nucl | agccatgtgactltcgtgtgtcggggcccggttggggttcaaacattccgcctggagaag |
| | | gacagaagatccacatacaatgatactgaagatgtgtctcaacctagtccatctgagtc |
| | | agaggccagattccgcattgactcagtaactgaaggaaatgccgggctttatcgctgcg |
| | | tctattataagccccctaaatggtctgaccagagtgacttcctggagttgctggtgaag |
| 71 | MGC17_EXON | EDLPRPSISAEEGTVIPLGSRLTFVCRGPVGVHTFRLERDRRSTY |
| | aa | NDTEDVSHPSPSESEARFRIDSVSEGNAGLYRCVYYKSPEWSK |
| | | QSDYLELLVK |
| 72 | MGC17_EXON | Gaagatctgcccagaccctccatctcggctgaggaaggcaccgtgattcccctgggg |
| | nucl | agccgtctgactttcgtgtgccggggcccggttggggttcacacattccgcctggagag |
| | | ggaccgtagatccacatacaatgatactgaagatgtgtctcaccctagtccatctgagtc |
| | | tgaggccagatttcgcattgactcagtgagtgaaggaaatgccgggctttatcgctgcgt |
| | | ctattataagtcccctgaatggtctaagcagagtgattacctggagctgctggtgaaa |
| 73 | MGC26_EXON | EDLPRPSISPEPATVIPLGSHVTIVCRGPVGVETFRLQKESRSLYN |
| | aa | DTEDVSQPSPSESEARFRIDSVSEGHGGLYRCLYYKSSKWSEQS |
| | | DYLEMLVK |
| 74 | MGC26_EXON | Gaagatctgcccagaccctccatctcgccggagccagccaccgtgatccccctggg |
| | nucl | gagccatgtgactatcgtgtgccggggcccggttggggttgaaacattccgcctgcaga |
| | | aggagagtagatccctgtacaatgacactgaagatgtgtctcaacctagtccatctgagt |
| | | cagaggccagattccgcattgactcagtaagtgaagggcatggcgggctttatcgctgc |
| | | ctctattataagtcttctaaatggtctgagcagagtgactacctggagatgctggtgaaa |
| 75 | MGC28/MGC33_EXON | EDLPRPTISAETGTVISLGSHVTFVCRGPLGVQTFRLERESRSRY |
| | aa | SETEDVSQVGPSESEARFRIDSVSEGNAGLYRCIYYKPPKWSEQ |
| | | SDYLELRVK |
| 76 | MGC28/MGC33_EXON | Gaagatctgcccagacccaccatctcggctgagacaggcaccgtgatctccctggg |
| | nucl | gagccatgtgactttcgtgtgccggggcccacttggggtgcaaacattccgcctggaga |
| | | gggagagtaggtccagatacagtgaaactgaagatgtgtctcaagttggtccatctgagt |
| | | cagaggccagattccgcattgactcagtgagtgaaggaaatgccgggctttatcgatgc |
| | | atctattacaaaccccctaaatggtctgagcagagtgactacctggagctgcgggtgaaa |
| 77 | MGC34_EXON | EDLPRPSISAEPGTVIPLGSHVTFVCRGPVGIHTFRLERESRSLYT |
| | aa | ETEDVTQVSPSESEARFRIESVTEGNAGLYRCVYYKPPKWSEQS |
| | | DYLELLVK |
| 78 | MGC34_EXON | Gaagatctgcccagaccctccatctcggctgagccaggcaccgtgatccccctggg |
| | nucl | gagtcatgtgaccttcgtgtgccggggcccggttgggattcacacattccgcctggaga |
| | | gggagagtagatccctatacactgaaactgaagatgtgactcaagtaagtccttctgagt |
| | | cagaggccagattccgcattgagtcagtaactgaaggaaatgccgggctttatcgctgc |
| | | gtctattataagccccctaaatggtctgagcagagtgactacctggagctgctggtgaaa |
| 79 | MGC35_EXON | EDLPRPSISAEPGSVIPLGSLVTFVCRGPVGVHTFRLERGWTYN |
| | aa | DTEDVSQAGPSESEARFRMDSVREGNAGLYRCIYYKPPKWSE |
| | | QSAYLELRVK |
| 80 | MGC35_EXON | Gaagatctgcccagaccctccatctcggctgagccaggctccgtgatccccctgggg |
| | nucl | agccttgtgactttcgtgtgccggggcccggttggggttcacacattccgcctcgagagg |
| | | gggtggacatacaacgacactgaagatgtgtctcaagctggtccatctgagtcagagg |
| | | ccagattccgcatggactcggtaagggaaggaaatgccgggctttatcgatgcatctatt |
| | | acaaaccccctaaatggtctgagcagagtgcctacctggaactgcgggtgaaa |
| 81 | MGC36_EXON | EEDLPRPSISAEPDTVIPLGSHVTFVCRGPVGVHTFRLERGWRY |
| | aa | NDTEDVSQAGPSESEARFRIDSVREGNAGLYRCIYYIAPKWSE |
| | | QSDYLELRVK |
| 82 | MGC36_EXON | Gaagaagatctgcccagaccctccatctcggctgagccagacaccgtaatccccct |
| | nucl | ggggagccatgtgactttcgtgtgccggggcccggttggggttcacacattccgcctgg |
| | | agagggggtggaggtacaacgacactgaagatgtgtctcaagctggtccatctgagtc |
| | | agaggccagattccgcattgactcggtaagggaaggaaatgccgggctttatcgatgc |
| | | atctattacatagcccctaaatggtctgagcagagtgactacctggagctgcgggtgaaa |
| 83 | MGD21_EXON | DLPRPSISAEPGTVIPLGSHVTFVCRGPVGVQTFRLERERNYLY |
| | aa | SDTEDVSQTSPSESEARFRIDSVNAGNAGLFRCIYYKSRKWSE |
| | | QSDYLELVVK |
| 84 | MGD21_EXON | Gatctgcccagaccctccatctcggctgagccgggcaccgtgatccccctggggag |
| | nucl | ccatgtgactttcgtgtgccggggcccggttggggttcaaacattccgcctggagaggg |
| | | agaggaattatttatacagtgatactgaagatgtgtctcaaactagtccatctgagtcgga |
| | | ggccagattccgcattgactcagtaaatgcaggcaatgccgggctttticgctgcatcta |
| | | ttacaagtcccgtaaatggtctgagcagagtgactacctggagctggtggtgaaa |
| 85 | MGD23_EXON | EDLPRPSLSAEPGTVIPLGSHVTFVCRGPAGVETERLERESRFTY |
| | aa | NDTEDVSQASPSESEARFRIDSVSEGNAGPYRCLYYKARKWSD |
| | | QSDYLELLVK |
| 86 | MGD23_EXON | Gaagatctgcccagaccctccctctcggctgaaccaggcaccgtgatccccctggg |
| | nucl | gagtcacgtgactttcgtgtgccggggcccggctggggtcgaaacattccgcctggag |
| | | agggagagtagattcacttacaacgatactgaagatgtgtctcaagcgagtccatctga |
| | | gtcagaggccagattccgcattgactcagtaagtgaaggaaatgccgggccttatcgct |
| | | gcctctattataaggcccgtaaatggtctgaccagagtgactacttggaattgctggtga |
| | | ag |
| 87 | MGD30_EXON | EKLPRPSISAEPGTVIPLGSRVTFVCRGPVGVQTERLERETSFTY |
| | aa | NDTEDVSQVSPSESEARFRIDSVSEGYAGPYRCVYYKAPKWSE |
| | | QSDYLDLLVK |
| 88 | MGD30_EXON | Gaaaaactgcccagaccctccatctcggctgagccgggcaccgtgatccccctggg |
| | nucl | gagccgtgtgactttcgtgtgccggggcccggttggggttcaaacattccgcctagaga |
| | | gggagactagctttacatataatgatactgaagatgtgtctcaggttagtccgtctgagtc |
| | | agaggccagattccgcattgactcagtgagtgagggatatgccgggccttatcgctgcg |
| | | tctattataaggcccctaagtggtccgagcagagtgactacctggacctgctggtgaaa |
| 89 | MGD33_EXON | EKLPRPSISAEPGTV1PLGSRVTFVCRGPVGVQTFRLERETRSTY |
| | aa | NDTEDVSQVSPSESEARFRIDSVSEGYAGPYRCVYYKAPKWSE |
| | | QSDYLDLLVK |
| 90 | MGD33_EXON | gaaaaactgcccagaccctccatctcggctgagccgggcaccgtgatccccctggg |
| | nucl | gagccgtgtgactttcgtgtgccggggcccggttggggttcaaacattccgcctagaga |
| | | gggagactagatctacatataatgatactgaagatgtgtctcaggttagtccgtctgagtc |
| | | agaggccagattccgcattgactcagtgagtgagggatatgccgggccttatcgctgcg |
| | | tctattataaggcccctaagtggtccgagcagagtgactacctggacctgctggtgaaa |
| 91 | MGD34_EXON | EDLPRPSISAEPGTVIPLGSHVTFVCRGPVGVQTERLERERNYL |
| | aa | YSDTEDVSQTSPSESEARFRIDSVNAGNAGLFRCIYYKSRKWSE |
| | | QSDYLELVVK |
| 92 | MGD34_EXON | Gaagatctgcccagaccctccatacggctgagccgggcaccgtgatccccaggg |
| | nucl | gagccatgtgactttcgtgtgccggggcccggttggggttcaaacattccgcctggaga |
| | | gggagaggaattatttatacagtgatactgaagatgtgtctcaaactagtccatctgagtc |
| | | ggaggccagattccgcattgactcagtaaatgcaggcaatgccgggctttttcgctgcat |
| | | ctattacaagtcccgtaaatggtctgagcagagtgactacctggagctggtggtgaaa |
| 93 | MGD35_EXON | NLPRPSLSAEPGTVIPLGSPVTFVCRGPVGVHTFRLERAGRSTY |
| | aa | NDTEDVSHPSPSESEARFRIDSVSEGNAGPYRCVYYKSSKWSEE |
| | | SYCLDLLVK |
| 94 | MGD35_EXON | aatttgcccagaccctccctctcggcggagccaggcaccgtgatccccctggggagc |
| | nucl | cctgtgactttcgtgtgccggggcccggttggggttcacacattccgcctggagagggc |
| | | gggtagatccacatacaatgatactgaagatgtgtctcatcctagtccatctgagtcaga |
| | | ggccagattccgcattgactcagtgagtgagggaaatgccgggccttatcgctgcgtct |
| | | attataagtcctctaaatggtccgaggagagttactgcctggacctgctggtcaaa |
| 95 | MGD39_EXON | DDLPRPSISAEPGTVIPLGSHVTFVCRGPIGVQTFRLERERRSLY |
| | aa | SDTEDVSQVSPFASEARFRIDSVSEGNAGPYRCIYYKDRKWSD |
| | | QSDYLELLVK |
| 96 | MGD39_EXON | Gacgatctgcccagaccctccatctcggctgagccaggcaccgtgatccccctggg |
| | nucl | gagccatgtgaccacgtgtgccggggcccaattggggttcaaacattccgcctggaga |
| | | gggagagaagatccttatacagtgatactgaagatgtgtctcaagttagtccatttgcgtc |
| | | agaggccagattccgcattgactcagtaagtgaaggaaatgccgggccatatcgctgc |
| | | atctattataaggaccggaaatggtctgaccagagtgactacctggagttgctggtgaaa |
| 97 | MGD41_EXON | EDLPRPSLSAEPGTVVPLGSHVTFVCRGPVGVQTFRLERESRST |
| | aa | YNDTEDVSQPSPFESEARFRIDSVSEGNAGPYRCIYYKSPKWS |
| | | DQSDYVELLVK |
| 98 | MGD41_EXON | Gaagatctgcccagaccctccctctcggctgagccaggcaccgtggtccccctggg |
| | nucl | gagccatgtgactttcgtgtgccggggcccggaggggttcaaacattccgcctggaga |
| | | gggagagcagatccacatacaatgatactgaagatgtgtctcaacctagtccatttgagt |
| | | cagaggccagatttcgcattgactcagtaagtgaaggaaatgccgggccttatcgctgc |
| | | atctattataagtcccctaaatggtctgaccagagtgactacgtggagttgctggtgaaa |
| 99 | MGD47_EXON | GDLPRPSISAEPGTAIPLGSQVTFVCRGPIGVQTFRLERESRALY |
| | aa | NDSEDVSQVSPSASEARFRIDSVSEGNAGPYRCIYYKARRWSD |
| | | QSDYLELLVK |
| 100 | MGD47_EXON | ggagatctgcccagaccctccatctcggctgagccaggcaccgcgatccccctggg |
| | nucl | gagccaagtgactttcgtgtgccggggcccaattggggttcaaacattccgcctggaga |
| | | gggagagtcgcgccttatataatgattctgaagatgtgtctcaagttagtccatctgcgtc |
| | | agaggccagattccgcattgactcagtaagtgaaggcaatgccgggccttatcgctgta |
| | | tctattataaggcccgcagatggtctgaccagagtgactatttggagttgttggtgaaa |
| 101 | MGD55_EXON | DDLPRPSISAEPGTVIPLGSHVTFVCRGPIGVQTFRLERESRSLY |
| | aa | SDTEDVSQVSPFASEARFRIDSVSEGNAGPYRCIYYKDRKWSD |
| | | QSDYLELLVK |
| 102 | MGD55_EXON | gacgatctgcccagaccctccatctcggctgagccaggcaccgtgatccccctgggg |
| | nucl | agccatgtgactttcgtgtgccggggcccaattggggttcaaacattccgcctggagag |
| | | ggagagtagatccttatacagtgatactgaagatgtgtctcaagttagtccatttgcgtca |
| | | gaggccagattccgcattgactcagtaagtgaaggaaatgccgggccatatcgctgca |
| | | tctattataaggaccggaaatggtctgaccagagtgactacctggagttgctggtgaaa |
| 103 | MGD56_EXON | KDLPRPSLSAEPGTVIPLGSHVTFVCRGPVGVQTFRLQRESRSL |
| | aa | YNDTEDVSHPSPSESEARFRIDSVSEGNAGPYRCVYYKSSKWS |
| | | EESDCLELLVK |
| 104 | MGD56_EXON | aaagatttgcccagaccctccctctcggctgagccaggcaccgtgatccccctgggg |
| | nucl | agtcatgtgactttcgtgtgccggggcccggttggggttcagacattccgcctgcagagg |
| | | gagagtagatccctttacaatgatactgaagatgtgtctcatcctagtccatctgagtcag |
| | | aggccagattccgcattgactcagtgagtgagggaaatgccgggccttatcgctgcgtc |
| | | tattataagtcctctaaatggtccgaggagagtgactgcctggagctgctggtcaaa |

Thus, it is preferred that the LAIR-1 fragment comprised by the protein according to the present invention has an amino acid sequence according to any of SEQ ID NOs 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101 and 103 or according to a functional sequence variant thereof as described herein. More preferably, the LAIR-1 fragment according to the present invention has an amino acid sequence according to SEQ ID NO: 83 or according to a functional sequence variant thereof. It is also preferred that the LAIR-1 fragment comprised by the protein according to the present invention has an amino acid sequence according to any of SEQ ID NOs 23, 25, 27, 29, 31, 33, 35, 37, 39, 41, 43, 45, 47, 49, 51, 53, 55, 57 and 59 or according to a functional sequence variant thereof as described herein. It is also preferred that the LAIR-1 fragment comprised by the protein according to the present invention has an amino acid sequence according to any of SEQ ID 61, 63, 65, 67, 69, 71, 73, 75, 77, 79, 81, 83, 85, 87, 89, 91, 93, 95, 97, 99, 101 and 103 or according to a functional sequence variant thereof as described herein.

Preferably, the LAIR-1 fragment comprised by the protein according to the present invention comprises at least the following mutations in comparison to native human LAIR-1: T67L, P107R, and N69S. More preferably, the LAIR-1 fragment comprised by the protein according to the present invention comprises at least the following mutations in comparison to native human LAIR-1: T67L, P107R, N69S and A77T. Even more preferably the LAIR-1 fragment comprised by the protein according to the present invention comprises at least the following mutations in comparison to native human LAIR-1: T67L, N69S, A77T, P106S, and P107R.

It is also preferred that the protein according to the present invention comprises more than one mutated LAIR-1 fragment as described herein, e.g. 2, 3, 4, 5, 6, 7, 8, 9, or 10 mutated LAIR-1 fragment as described. Preferably, if the protein according to the present invention comprises more than one mutated LAIR-1 fragment, the LAIR-1 fragments are linked by a linker as described herein, for example GGGGS. Such a protein according to the present invention comprising more than one mutated LAIR-1 fragment, optionally linked by a linker as described herein, for example GGGGS, is a fusion protein.

In particular, the LAIR-1 fragment comprised by the protein according to the present invention, and thus the protein according to the present invention, binds to a Plasmodium falciparum variant surface antigen. It is thus preferred that the LAIR-1 fragment comprised by the protein according to the present invention, and thus the protein according to the present invention, binds to an antigen on Plasmodium falciparum-infected erythrocytes.

Preferably, the protein according to the present invention binds to a RIFIN, preferably to a type A RIFIN.

As used herein, the term “Plasmodium falciparum variant surface antigen” includes PfEMP1 (P. falciparum erythrocyte membrane protein 1), RIFIN (repetitive interspersed family proteins), STEVOR (sub-telomeric variable open reading frame proteins) and SURFIN (surface-associated interspersed gene family proteins).

A “RIFIN” as used herein refers to a protein of the RIFIN family (repetitive interspersed family proteins). In addition to proteins, which are classified as RIFINs, the skilled person may easily determine whether any (unknown) protein is a RIFIN by use of appropriate computer programs, for example “RSpred”, which is freely accessible under http://www.bioinfo.ifm.liu.se/ and described by Joannin N. et al., 2011: RSpred, a set of Hidden Markov Models to detect and classify the RIFIN and STEVOR proteins of Plasmodium falciparum. BMC genomics 12:119.

RIFINs (repetitive interspersed family proteins) represent a second family of antigens found at the surface of IEs. These polypeptides are encoded by 150 rif genes and comprise the largest family of antigenically variable molecules in P. falciparum. Although the function of RIFINs is unknown, it has been shown that they are resistant to enzyme degradation and upregulated in rosetting parasites and it has been speculated that they contribute to the resetting of IEs with non-infected erythrocytes and to sequestration of P. falciparum.

Preferably, the LAIR-1 fragment comprised by the protein according to the present invention, and thus the protein according to the present invention, binds to the “second variable (V2) domain” of a RIFIN and/or to the “N-terminal semi-conserved domain” (also referred to as “C1” or “Constant Region 1”) of a RIFIN. More preferably the protein according to the present invention (and in particular the LAIR-1 fragment comprised by that protein) binds to the “second variable (V2) domain” of a RIFIN, but not to the “N-terminal semi-conserved (C1) domain” of a RIFIN.

It is also preferred that the protein according to the present invention, in particular the LAIR-1 fragment comprised by that protein, binds to the “second variable (V2) domain” of a type A RIFIN and/or to the “N-terminal semi-conserved domain” of a type A RIFIN. More preferably the protein according to the present invention (and in particular the LAIR-1 fragment comprised by that protein) binds to the “second variable (V2) domain” of an A-type RIFIN, but not to the “N-terminal semi-conserved (C1) domain” of a RIFIN, in particular of an A-type RIFIN.

RIFINs carry a semi-conserved domain and cysteine-rich regions at the N-terminus, while the C-terminal half is highly polymorphic. RIFINs are described as small polypeptides comprising (in the direction from N- to C-terminus):
(1) a putative signal peptide (SP),
(2) a first variable domain (V1),
(3) a plasmodium export element (PEXEL),
(4) an N-terminal semi-conserved domain (C1, also referred to as “constant region 1”),
(5) a hydrophobic patch, which is proposed to be a transmembrane domain (TM1),
(6) a second variable domain, also known as hypervariable domain (V2),
(7) a (second) transmembrane domain (TM2), and
(8) a C-terminal conserved domain (C2)

as described for example by Joannin N. et al., 2008, BMC genomics 9:19 (FIG. 1 and corresponding description) and by Templeton T. J., 2009, Molecular & Biochemical Parasitology 166: 109-116. By using this literature, the skilled person can easily assign the above protein domains (1)-(8) to any RIFIN. Moreover, the skilled person is also aware of databases for protein domain prediction, for example “NCBI conserved domain search” (www.ncbi.nlm.nih.gov/Structure/cdd/wrpsb.cgi), “SMART” (smartembl-heidelberg.de/), InterPro protein (www.ebi.ac.uk/interpro/), “PredictProtein” (https://www.predictprotein.org/), and the like.

The second variable (V2) domain (also known as “hypervariable domain”; (6)) comprises approximately 170 polymorphic residues and is predicted to be exposed on the cell surface (i.e. extracellular localization). A role of the second variable (V2) domain (hypervariable domain; (6)) in antigenic variation was suggested. However, the actual orientation of RIFINs within membrane is still debatable, since only the C-terminal transmembrane domain (7) is widely accepted as transmembrane domain, whereas the more N-terminal “hydrophobic patch” (5) was initially suggested to be a second transmembrane domain, which is, however, under discussion (for review see Templeton T. J., 2009, Molecular & Biochemical Parasitology 166: 109-116, in particular FIG. 3 suggesting different models). Depending on whether the hydrophobic patch (5) indeed constitutes a second transmembrane domain the N-terminus of the RIFINS including the N-terminal semi-conserved domain ((4); C1, also referred to as “constant region 1”) is located either intracellularly or extracellularly (cf. Templeton T. J., 2009, Molecular & Biochemical Parasitology 166: 109-116, in particular FIG. 3 suggesting different models).

Particularly preferably, the LAIR-1 fragment comprised by the protein according to the present invention, and thus the protein according to the present invention, binds to RIFIN PF3D7_1400600 and/or to RIFIN PF3D7_1040300, more preferably to the second variable (V2) domain and/or to the N-terminal semi-conserved domain of RIFIN PF3D7_1400600 and/or to the second variable (V2) domain and/or to the N-terminal semi-conserved domain of RIFIN PF3D7_1040300. Even more preferably, the LAIR-1 fragment comprised by the protein according to the present invention, and thus the protein according to the present invention, binds (i) to the second variable (V2) domain of RIFIN PF3D7_1400600, but not to the N-terminal semi-conserved domain of RIFIN PF3D7_1400600, and/or (ii) to the second variable (V2) domain of RIFIN PF3D7_1040300, but not to the N-terminal semi-conserved domain of RIFIN PF3D7_1040300.

The amino acid sequence of RIFIN PF3D7_1400600, as well as the nucleic acid sequence encoding it, is shown below in Table 2. Moreover, Table 2 shows also the amino acid sequences of the second variable (V2) domain and of the N-terminal semi-conserved domain of RIFIN PF3D7_1400600. The amino acid sequence of RIFIN PF3D7_1040300, as well as the nucleic acid sequence encoding it, is shown below in Table 2. Moreover, Table 2 shows also the amino acid sequences of the second variable (V2) domain and of the N-terminal semi-conserved domain of RIFIN PF3D7_1040300.

TABLE 2
Amino acid sequences and nucleic acid sequences of RIFINs
PF3D7_1400600 and PF3D7_1040300.

| SEQ | | |
|---|---|---|
| ID | | |
| NO | Description | Sequence |
| 105 | PF3D7_1400600 | MKDHYINILLFALPLNILVYNQRNYYITPRHTETNRSLCE |
| | aa | CELYSPTNYDSDPEMKRVMQQFVDRTTQRFHEYDESLQSK |
| | | RKQCKDQCDKEIQKIILKDKIEKEFTEKLSTLQTDITTKD |
| | | IPTCVCEKSLADKMEKVCLKCAQNLGGIVAPSTGVLGEIA |
| | | ALAVNAWKTTALKNAIAAAQKAGDAAGKIAGESKGVETII |
| | | GILEQYYSIYELKGTPLKSFFATTHYTDISNIATVIDTEL |
| | | NTSCGLNSLANQAICGLRTKLGLVAKPGQVMVTQKEAITK |
| | | MITNVVHKSEITAEAAKTEVAATKTAAAIKMNTEAIEAAT |
| | | TPYYTPIIASIVAIVVIVLIMVIIYLILRYRRKKKMKKKL |
| | | QYIKLLN* |
| 106 | PF3D7_1400600 | ATGAAAGACCATTATATTAATATATTATTGTTTGCTCTTC |
| | nucl | CATTAAATATATTGGTATATAATCAAAGGAACTATTACAT |
| | | TACACCACGTCATACAGAAACCAACAGATCTTTATGTGAA |
| | | TGTGAATTATATTCACCTACGAACTATGATAGTGATCCCG |
| | | AAATGAAAAGGGTAATGCAACAATTTGTGGATCGTACAAC |
| | | ACAACGATTTCACGAATATGATGAAAGTTTGCAAAGTAAA |
| | | CGAAAGCAATGCAAAGATCAATGCGATAAAGAAATCCAAA |
| | | AAATTATATTAAAAGATAAAATCGAAAAGGAATTTACAGA |
| | | AAAATTATCAACATTACAAACAGATATAACGACTAAAGAC |
| | | ATACCCACCTGTGTTTGCGAAAAATCCTTGGCGGACAAAA |
| | | TGGAAAAAGTATGCTTGAAATGTGCACAAAATTTGGGAGG |
| | | TATTGTTGCACCCTCTACAGGAGTATTAGGCGAAATTGCT |
| | | GCACTTGCTGTAAATGCCTGGAAAACTACGGCACTTAAGA |
| | | ACGCTATTGCGGCAGCTCAAAAAGCAGGTGATGCGGCCGG |
| | | TAAAATTGCGGGGGAATCCAAGGGTGTTGAAACAATTATT |
| | | GGAATATTAGAACAATATTACTCTATATATGAGTTAAAAG |
| | | GAACACCATTGAAATCCTTTTTTGCTACAACGCATTATAC |
| | | TGATATCTCAAATATTGCTACTGTTATTGATACGGAATTG |
| | | AATACGTCTTGTGGGTTGAATTCCTTAGCTAATCAGGCTA |
| | | TTTGCGGTCTTCGTACGAAATTAGGTCTTGTTGCAAAACC |
| | | TGGTCAAGTTATGGTTACACAGAAAGAAGCTATAACAAAG |
| | | ATGATAACCAACGTTGTTCATAAATCTGAAATTACTGCTG |
| | | AAGCTGCAAAGACTGAGGTGGCTGCAACTAAAACAGCAGC |
| | | AGCTATAAAGATGAACACAGAAGCTATAGAAGCTGCAACT |
| | | ACTCCTTACTATACTCCTATAATAGCATCCATCGTTGCAA |
| | | TAGTGGTCATAGTTTTAATTATGGTGATAATTTATTTGAT |
| | | TTTACGTTATCGAAGAAAAAAAAAAATGAAGAAAAAACTC |
| | | CAATATATAAAATTATTAAATTAA |
| 107 | PF3D7_1040300 | MKFNYTNIILFSLSLNILLLSSRVYNKRNHKSIILHTSNE |
| | aa | NPIKTHRSLCECELYSPTNYDSDPEMKRVMQQFHDRTTQR |
| | | FHEYDERMKTTRQECKEQCDKEIQKIILKDRLEKELMDKF |
| | | ATLHTDIQSDAIPTCVCEKSLADKTEKFCLNCGVQLGGGV |
| | | LQASGLLGGIGQLGLDAWKAAALVTAKELAEKAGAAAGLK |
| | | AGDIHGMKIVIEGLKALKVDTLKSGIFNSFVNNSHYTEVT |
| | | GLAIAIDTEMNEVCSATYIGIHPICVVREKLGVIPKAGGT |
| | | MVKQKDAITNVLKQALEKATQSAEALSETTAEDVAAKLTA |
| | | QKTGAINTIFMSNQTAIIASIVAIVVIVLIMVIIYLILRY |
| | | RRKKKMKKKLQYIKLLEE |
| 108 | PF3D7_1040300 | ATGAAGTTCAATTACACTAATATAATATTATTTTCCCTTT |
| | nucl | CATTAAATATATTGTTATTATCATCACGGGTATACAATAA |
| | | AAGGAATCATAAAAGCATTATACTTCATACATCAAACGAA |
| | | AACCCAATAAAAACACATAGATCATTATGCGAATGCGAAT |
| | | TATATTCACCTACGAACTATGATAGTGATCCCGAAATGAA |
| | | AAGGGTAATGCAACAATTTCATGATCGTACAACACAACGA |
| | | TTTCACGAATACGACGAAAGGATGAAAACTACACGCCAAG |
| | | AATGTAAAGAACAATGCGATAAAGAAATACAAAAAATTAT |
| | | TTTAAAAGACAGATTAGAAAAAGAATTAATGGACAAATTT |
| | | GCCACACTACACACAGATATACAAAGTGATGCTATTCCAA |
| | | CATGTGTTTGCGAAAAGTCGTTAGCAGATAAAACAGAAAA |
| | | ATTTTGTCTGAACTGTGGGGTGCAACTAGGAGGTGGTGTG |
| | | TTGCAAGCTTCGGGTTTATTAGGAGGAATTGGTCAACTTG |
| | | GGCTAGATGCATGGAAAGCAGCCGCGTTGGTAACTGCTAA |
| | | GGAACTTGCCGAAAAAGCCGGTGCTGCAGCAGGTCTTAAA |
| | | GCAGGTGATATCCATGGTATGAAAATAGTTATTGAAGGAT |
| | | TAAAAGCATTGAAAGTAGATACATTAAAATCTGGAATATT |
| | | TAATTCCTTTGTTAATAACAGCCATTATACTGAAGTCACA |
| | | GGGCTTGCTATTGCTATTGATACTGAAATGAATGAGGTGT |
| | | GTTCAGCGACGTATATTGGTATTCATCCTATCTGCGTTGT |
| | | TCGTGAGAAATTAGGTGTAATACCAAAGGCTGGTGGAACA |
| | | ATGGTTAAACAGAAAGATGCTATAACAAATGTGTTAAAGC |
| | | AAGCTCTTGAAAAAGCTACACAAAGTGCTGAAGCACTTTC |
| | | TGAGACTACTGCTGAAGACGTTGCTGCTAAACTCACAGCT |
| | | CAAAAGAGGGGTGCGATAAATACTATATTTATGAGTAATC |
| | | AGACTGCTATTATTGCTTCCATCGTTGCAATAGTAGTTAT |
| | | AGTTTTAATTATGGTGATAATATATTTAATTTTACGTTAT |
| | | CGACGAAAAAAAAAAATGAAGAAAAAACTCCAATATATCA |
| | | AATTATTAGAAGAATAG |
| 639 | PF3D7_1400600 | IAALAVNAWKTTALKNAIAAAQKAGDAAGKIAGESKGVETII |
| | second variable | GILEQYYSIYELKGTPLKSFFATTHYTDISNIATVIDTELNTSCGL |
| | (V2) domain | NSLANQAICGERTKLGLVAKPGQVMVTQKEAITKMITNVVH |
| | | KSEITAEAAKTEVAATKTAAAIKMNTEAIEAATTPYYT |
| 640 | PF3D7_1400600 | CELYSPTNYDSDPEMKRVMQQFVDRTTQRFHEYDESLQSKR |
| | N-terminal semi- | KQCKDQCDKEIQKIILKDKIEKEFTEKLSTLQTDITTKDIPTCVC |
| | conserved (C1) | EKSLADKMEKVCLKCAQNLGGIVAPSTGVLG |
| | domain | |
| 641 | PF3D7_1040300 | IGQLGLDAWKAAALVTAKELAEKAGAAAGLKAGDIHGMKIV |
| | second variable | IEGLKALKVDTLKSGIFNSFVNNSHYTEVTGLAIAIDTEMNEVC |
| | (V2) domain | SATYIGIHPICVVREKLGVIPKAGGTMVKQKDAITNVLKQALE |
| | | KATQSAEALSETTAEDVAAKLTAQKTGAINTIFMSNQT |
| 642 | PF3D7_1040300 | CELYSPTNYDSDPEMKRVMQQFHDRTTQRFHEYDERMKTT |
| | N-terminal semi- | RQECKEQCDKEIQKIILKDRLEKELMDKFATLHTDIQSDAIPTC |
| | conserved (C1) | VCEKSLADKTEKFCLNCGVQLGGGVLQASGLLG |
| | domain | |

Thus, it is particularly preferred that the LAIR-1 fragment comprised by the protein according to the present invention, and thus the protein according to the present invention, binds to a protein comprising an amino acid sequence according to SEQ ID NO: 105 or a functional sequence variant thereof and/or to a protein comprising an amino acid sequence according to SEQ ID NO: 107 or a functional sequence variant thereof. Most preferably, the protein according to the present invention, binds to a protein comprising an amino acid sequence according to SEQ ID NO: 105 or a functional sequence variant thereof and to a protein comprising an amino acid sequence according to SEQ ID NO: 107 or a functional sequence variant thereof.

Binding to a Plasmodium falciparum variant surface antigen, preferably to a RIFIN, more preferably to RIFIN PF3D7_1400600, may be easily determined. For example, 1) a RIFIN may be expressed on the surface of cell of mammalian cells (293 Expi) used for transfection and they are then stained with the protein in question, e.g. with the (exemplary) antibodies and/or the (“exon”-)fusion proteins as described herein; or 2) a RIFIN may be expressed as fusion protein in mammalian cells (293 Expi) and they are then tested if they bind to the protein in question, e.g. to the (exemplary) antibodies and/or the (“exon”-)fusion proteins as described herein by ELISA.

Methods for testing proteins, in particular (monoclonal and/or polyclonal) antibodies, for their binding affinities are well known in the art. One possibility among others is to characterize the binding affinity of an antibody by means of a sandwich ELISA by using the target peptide as well as negative controls (e.g. the same peptide with L-amino acids only). The ELISA limit can—without being limited thereto—be calculated on blank replicates as follows:

ELISA limit=average (negative control)+(3×standard deviation of negative control).

If the sample value is less or equal to the ELISA limit the tested antibody may be considered to have no affinity to the target peptide. If the sample value exceeds the ELISA limit the tested antibody may be considered to exhibit affinity to the target peptide. Moreover, the higher the sample value, the stronger is the affinity of the tested antibody for the target.

Preferably, the protein according to the present invention limits, in particular neutralizes, infection by Plasmodium falciparum. Preferably, the protein according to the present invention prevents the pathology of malaria, in particular by preventing rosetting and adhesion to endothelia. As used herein, a “neutralizing” means to reduce the pathogen load by opsonizing IEs and promoting their phagocytosis or by blocking adhesion of IEs to non-infected erythrocytes or to endothelia and thus impede or interfere with, the ability of a pathogen, in particular Plasmodium falciparum, to cause severe spread malaria infection in a host. Neutralization may be assessed by an opsonization assay, as known to the person skilled in the art.

In the following a non-limiting example of an opsonization assay is given to illustrate the principle:
(1) P. falciparum-infected human erythrocytes are exposed to a DNA dye to label the parasites;
(2) the P. falciparum-IEs are cultured under appropriate conditions with human CD14+ monocytese.g. for 1 hour at 37° C. in 5% CO₂; and
(3) the cultures are analyzed by flow cytometry and the amount of CD14+ monocytes that have opsonized the labeled-IEs can be determined.

The effects measured are usually dose-dependent: The higher the protein concentration, the stronger the biological effect measured in the assay. Depending on the neutralizing character of the protein, the amount of opsonized IEs may vary, e.g. a protein, in particular an antibody, of significant neutralizing character will require lower amounts (of the protein/antibody) to be added for, e.g., achieving the same amount of neutralization of the target effect in the assay.

Preferably, the protein according to the present invention comprising a mutated LAIR-1 fragment as described above does not bind to collagen. Binding to collagen may be assessed by expression of the protein in question in a mammalian cell, e.g. in HEK293 cells, and assessing binding to collagen by ELISA, e.g. using ELISA plates coated with collagen, in particular Collagen type 1.

To this end, the mutated LAIR-1 fragment according to the present invention preferably comprises the mutation P107R, which abolishes the binding ability of LAIR-1 to collagen.

Preferably, the protein according to the present invention comprising a mutated LAIR-1 fragment as described above is used in the prevention and/or treatment of malaria, preferably of P. falciparum-malaria. Thereby, the term “prevention” comprises the prevention in a subject, which does not (yet) show symptoms of malaria as well as the prevention by decreasing the transmission of P. falciparum.

The protein according to the present invention may occur as such in nature, for example as an antibody isolated from a human subject, or it may be a recombinant protein. The term “recombinant” as used herein means that the protein does not occur naturally. Preferably the protein according to the present invention is a recombinant protein.

Further components of the protein according to the present invention

The protein according to the present invention preferably comprises one or more further components in addition to the mutated LAIR-1 fragment as described above. Such a further component of the protein according to the present invention may also be a protein or a (poly)peptide or the further component may be a molecule of other chemical nature, i.e. different from a protein or a polypeptide. Thereby, the term “molecule” refers to a group of two or more atoms held together by a chemical bond.

For example, the one or more further component(s) of the protein may be a label. Labels may comprise radioactive labels, i.e. radioactive phosphorylation or a radioactive label with sulphur, hydrogen, carbon, nitrogen, etc.; colored dyes (e.g. digoxygenin, etc.); fluorescent groups (e.g. fluorescein, rhodamine, flourochrome proteins as defined below, etc.); chemoluminescent groups; or combination of these labels. Labeled proteins, in particular labeled antibodies, may be employed in a wide variety of assays, employing a wide variety of labels. Detection of the formation of an antibody-antigen complex between an antibody of the invention and an epitope of interest on a RIFIN can be facilitated by attaching a detectable substance to the protein, in particular to the antibody. Suitable detection means include the use of labels such as radionuclides, enzymes, coenzymes, fluorescers, chemiluminescers, chromogens, enzyme substrates or co-factors, enzyme inhibitors, prosthetic group complexes, free radicals, particles, dyes, and the like. Examples of suitable enzymes include horseradish peroxidase, alkaline phosphatase, f3-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material is luminol; examples of bioluminescent materials include luciferase, luciferin, and aequorin; and examples of suitable radioactive material include 125I, 131I, 35S, or 3H. Such labeled reagents may be used in a variety of well-known assays, such as radioimmunoassays, enzyme immunoassays, e.g., ELISA, fluorescent immunoassays, and the like. Labeled antibodies according to the present invention may be thus be used in such assays for example as described in U.S. Pat. No. 3,766,162; U.S. Pat. No. 3,791,932; U.S. Pat. No. 3,817,837; and U.S. Pat. No. 4,233,402. In addition, linkers may be used between the labels and the proteins, in particular the antibodies, of the invention, e.g., as described in U.S. Pat. No. 4,831,175. Proteins, in particular antibodies, according to the present invention may be directly labeled with radioactive iodine, indium, yttrium, or other radioactive particle known in the art, e.g., as described in U.S. Pat. No. 5,595,721.

Preferably, the protein according to the present invention may comprise a fluorochrome protein, in particular to a fluorochrome protein which can be activated such as to emit a fluorescence signal. Thereby, the protein according to the present invention comprising the LAIR-1 fragment according to the present invention and a fluorochrome protein is preferably provided as fusion protein. Preferably, the fluorochrome protein is selected from any fluorescent protein, e.g. from a group comprising the Green Fluorescent Protein (GFP), derivatives of the Green Fluorescent Protein (GFP), e.g. EGFP, AcGFP, TurboGFP, Emerald, Azami Green, the photo activatable-GFP (PA-GFP), or Blue Fluorescent Protein (BFP) including EBFP, Sapphire, T-Sapphire, or Cyan Fluorescent Proteins (CFP) including the enhanced cyan fluorescent protein (ECFP), mCFP, Cerulan, CyPet, or Yellow Fluorescent Proteins (YFP), including Topaz, Venus, mCitrine, Ypet, PhiYFP, mBanana, the yellow shifted green fluorescent protein (Yellow GFP), the enhanced yellow fluorescent protein (EYFP), or Orange and Red Flourescent Proteins (RFP) including Kusibara Orange, mOrange, dTomato-Tandem, DsRed-Monomer, mTangerine, mStrawberry, monomeric red fluorescent protein (mRFP1) (also designated herein as mRFP), mCherry, mRaspberry, HcRed-Tandem,mPlum, as well as optical highlighters selected from PA-GFP, CoralHue Dronpa (G), PS-CFP (C), PS-CFP (G), mEosFP (G), mEosFP (G), or other monomeric fluorescent proteins such as or the kindling fluorescent protein (KFP1), aequorin, the autofluorescent proteins (AFPs), or the fluorescent proteins JRed, TurboGFP, PhiYFP and PhiYFP-m, tHc-Red (HcRed-Tandem), PS-CFP2 and KFP-Red (as available from EVRΩGEN, see also www.evrogen.com), or other suitable fluorescent proteins.

Additionally and/or alternatively, the protein according to the present invention may be comprised by or attached to, for example, a drug for delivery to a treatment site. A protein, in particular an antibody, according to the present invention may be conjugated to a therapeutic moiety such as a cytotoxin, a therapeutic agent, or a radioactive metal ion or radioisotope. Examples of radioisotopes include, but are not limited to, I-131, I-123, I-125, Y-90, Re-188, Re-186, At-211, Cu-67, Bi-212, Bi-213, Pd-109, Tc-99, 1n-111, and the like. Such antibody conjugates can be used for modifying a given biological response; the drug moiety is not to be construed as limited to classical chemical therapeutic agents. For example, the drug moiety may be a protein or polypeptide possessing a desired biological activity. Such proteins may include, for example, a toxin such as abrin, ricin A, pseudomonas exotoxin, or diphtheria toxin.

Techniques for conjugating such therapeutic moiety to proteins, in particular to antibodies, are well known. See, for example, Arnon et al. (1985) “Monoclonal Antibodies for Immunotargeting of Drugs in Cancer Therapy,” in Monoclonal Antibodies and Cancer Therapy, ed. Reisfeld et al. (Alan R. Liss, Inc.), pp. 243-256; ed. Hellstrom et al. (1987) “Antibodies for Drug Delivery,” in Controlled Drug Delivery, ed. Robinson et al. (2d ed; Marcel Dekker, Inc.), pp. 623-653; Thorpe (1985) “Antibody Carriers of Cytotoxic Agents in Cancer Therapy: A Review,” in Monoclonal Antibodies ‘84: Biological and Clinical Applications, ed. Pinchera et al. pp. 475-506 (Editrice Kurtis, Milano, Italy, 1985); “Analysis, Results, and Future Prospective of the Therapeutic Use of Radiolabeled Antibody in Cancer Therapy,” in Monoclonal Antibodies for Cancer Detection and Therapy, ed. Baldwin et al. (Academic Press, New York, 1985), pp. 303-316; and Thorpe et al. (1982) Immunol. Rev. 62:119-158.

Alternatively, a protein, in particular an antibody, according to the present invention can be conjugated to a second antibody, or antibody fragment thereof, to form an antibody heteroconjugate as described in U.S. Pat. No. 4,676,980.

Proteins, e.g. antibodies, of the invention may also be attached to a solid support. Additionally, proteins, in particular antibodies, of the invention, can be chemically modified by covalent conjugation to a polymer to, for example, increase their circulating half-life. Examples of polymers, and methods to attach them to peptides, are shown in U.S. Pat. No. 4,766,106; U.S. Pat. No. 4,179,337; U.S. Pat. No. 4,495,285 and U.S. Pat. No. 4,609,546. In some embodiments the polymers may be selected from polyoxyethylated polyols and polyethylene glycol (PEG). PEG is soluble in water at room temperature and has the general formula: R—(O—CH2-CH2)n-O—R where R can be hydrogen, or a protective group such as an alkyl or alkanol group. Preferably, the protective group may have between 1 and 8 carbons. For example, the protective group is methyl. The symbol n is a positive integer. In one embodiment n is between 1 and 1,000. In another embodiment n is between 2 and 500. Preferably, the PEG has an average molecular weight between 1,000 and 40,000, more preferably the PEG has a molecular weight between 2,000 and 20,000, even more preferably the PEG has a molecular weight between 3,000 and 12,000. Furthermore, PEG may have at least one hydroxy group, for example the PEG may have a terminal hydroxy group. For example, it is the terminal hydroxy group which is activated to react with a free amino group on the inhibitor. However, it will be understood that the type and amount of the reactive groups may be varied to achieve a covalently conjugated PEG/protein of the present invention. Water-soluble polyoxyethylated polyols are also useful in the present invention. They include polyoxyethylated sorbitol, polyoxyethylated glucose, polyoxyethylated glycerol (POG), and the like. In one embodiment, POG is used. Without being bound by any theory, because the glycerol backbone of polyoxyethylated glycerol is the same backbone occurring naturally in, for example, animals and humans in mono-, di-, triglycerides, this branching would not necessarily be seen as a foreign agent in the body. POG may have a molecular weight in the same range as PEG. Another drug delivery system that can be used for increasing circulatory half-life is the liposome. Methods of preparing liposome delivery systems are known to one of skill in the art. Other drug delivery systems are known in the art and are described in, for example, referenced in Poznansky et al. (1980) and Poznansky (1984).

Linkage of the components in the protein according to the present invention

In the protein according to the present invention, the LAIR-1 fragment is in particular covalently linked to one or more of the other component(s) comprised by the protein according to the present invention, preferably the linkage of all components of the protein according to the present invention is a covalent linkage.

A “covalent linkage” (also covalent bond), as used in the context of the present invention, refers to a chemical bond that involves the sharing of electron pairs between atoms. A “covalent linkage” (also covalent bond) in particular involves a stable balance of attractive and repulsive forces between atoms when they share electrons. For many molecules, the sharing of electrons allows each atom to attain the equivalent of a full outer shell, corresponding to a stable electronic configuration. Covalent bonding includes many kinds of interactions, including for example σ-bonding, π-bonding, metal-to-metal bonding, agostic interactions, and three-center two-electron bonds.

Preferably, in the protein according to the present invention, the components, e.g. the LAIR-1 fragment and one or more further components, are covalently linked by chemical coupling in any suitable manner known in the art, such as cross-linking methods. However, attention is drawn to the fact that many known chemical cross-linking methods are non-specific, i.e., they do not direct the point of coupling to any particular site on the components or on the LAIR-1 fragment. Thus, the use of non-specific cross-linking agents may attack functional sites or sterically block active sites, rendering the fused components of the molecule according to the present invention biologically inactive. It is referred to the knowledge of the skilled artisan to block potentially reactive groups by using appropriate protecting groups. Alternatively, the use of the powerful and versatile oxime and hydrazone ligation techniques, which are chemo-selective entities that can be applied for the cross-linking of the components including the LAIR-1 fragment may be employed. This linking technology is described e.g. by Rose et al. (1994), JACS 116, 30.

Coupling specificity can be increased by direct chemical coupling to a functional group found only once or a few times in one of the further component(s) or of the LAIR-1 fragment comprised by the protein according to the present invention, which functional group is to be cross-linked to the LAIR-1 fragment comprised by the protein according to the present invention or to the another of the component(s). As an example, the cystein thiol group may be used. Also, for example, if a further component or the mutated LAIR-1 fragment comprised by the protein according to the present invention contains no lysine residues, a cross-linking reagent specific for primary amines will be selective for the amino terminus of the respective component. Alternatively, cross-linking may also be carried out via the side chain of a glutamic acid residue placed at the N-terminus of the peptide such that a amide bond can be generated through its side-chain. Therefore, it may be advantageous to link a glutamic acid residue to the N-terminus of a further component or the mutated LAIR-1 fragment comprised by the protein according to the present invention. However, if a cysteine residue is to be introduced into a further component or the mutated LAIR-1 fragment comprised by the protein according to the present invention, introduction at or near its N- or C-terminus is preferred. Conventional methods are available for such amino acid sequence alterations based on modifications of a further component or the mutated LAIR-1 fragment comprised by the protein according to the present invention by either adding one or more additional amino acids, e.g. inter alia an cystein residue, to the translocation sequence or by substituting at least one residue of the translocation sequence(s) being comprised in the respective component. In case a cystein side chain is used for coupling purposes, a further component or the mutated LAIR-1 fragment comprised by the protein according to the present invention has preferably one cystein residue. Any second cystein residue should preferably be avoided and can, optionally, be replaced when they occur in the respective component comprised by the molecule according to the present invention. When a cysteine residue is replaced in the original sequence of a further component or the mutated LAIR-1 fragment comprised by the protein according to the present invention, it is typically desirable to minimize resulting changes in the peptide folding of the respective component. Changes in folding are minimized when the replacement is chemically and sterically similar to cysteine. Therefore, serine is preferred as a replacement for cystein.

Coupling of a further component and the mutated LAIR-1 fragment comprised by the protein according to the present invention can be accomplished via a coupling or conjugating agent including standard peptide synthesis coupling reagents such as HOBt, HBTU, DICI, TBTU. There are several intermolecular cross-linking agents which can be utilized, see for example, Means and Feeney, Chemical Modification of Proteins, Holden-Day, 1974, pp. 39-43. Among these reagents are, for example, N-succinimidyl 3-(2-pyridyldithio)propionate (SPDP) or N,N′-(1,3-phenylene)bismaleimide; N,N′-ethylene-bis-(iodoacetamide) or other such reagent having 6 to 11 carbon methylene bridges; and 1,5-difluoro-2,4-dinitrobenzene. Other cross-linking agents useful for this purpose include: p,p′-difluoro-m,m′-dinitrodiphenylsulfone; dimethyl adipimidate; phenol-1,4-disulfonylchloride; hexamethylenediisocyanate or diisothiocyanate, or azophenyl-p-diisocyanate; glutaraldehyde and disdiazobenzidine. Cross-linking agents may be homobifunctional, i.e., having two functional groups that undergo the same reaction. A preferred homobifunctional cross-linking agent is bismaleimidohexane (BMH). BMH contains two maleimide functional groups, which react specifically with sulfhydryl-containing compounds under mild conditions (pH 6.5-7.7). The two maleimide groups are connected by a hydrocarbon chain. Therefore, BMH is useful for irreversible cross-linking of proteins (or polypeptides) that contain cysteine residues. Cross-linking agents may also be heterobifunctional. Heterobifunctional cross-linking agents have two different functional groups, for example an amine-reactive group and a thiol-reactive group, that will cross-link two proteins having free amines and thiols, respectively. Examples of heterobifunctional cross-linking agents are Succinimidyl-4-(N-maleimidomethyl)-cyclohexane-1-carboxylate (SMCC), m-maleimidobenzoyl-N-hydroxysuccinimide ester (MBS), and succinimide 4-(p-maleimidophenyl)butyrate (SMPB), an extended chain analog of MBS. The succinimidyl group of these cross-linkers reacts with a primary amine, and the thiol-reactive maleimide forms a covalent bond with the thiol of a cysteine residue. Because cross-linking agents often have low solubility in water, a hydrophilic moiety, such as a sulfonate group, may be added to the cross-linking agent to improve its water solubility. Sulfo-MBS and sulfo-SMCC are examples of cross-linking agents modified for water solubility. Many cross-linking agents yield a conjugate that is essentially non-cleavable under cellular conditions. Therefore, some cross-linking agents contain a covalent bond, such as a disulfide, that is cleavable under cellular conditions. For example, Traut's reagent, dithiobis (succinimidylpropionate) (DSP), and N-succinimidyl 3-(2-pyridyldithio)propionate (SPDP) are well-known cleavable cross-linkers. The use of a cleavable cross-linking agent permits the further component and the mutated LAIR-1 fragment comprised by the protein according to the present invention comprised by the molecule according to the present invention to separate from each other after delivery into the target cell. For this purpose, direct disulfide linkage may also be useful. Chemical cross-linking may also include the use of spacer arms. Spacer arms provide intramolecular flexibility or adjust intramolecular distances between conjugated moieties and thereby may help preserve biological activity. A spacer arm may be in the form of a protein (or polypeptide) moiety that includes spacer amino acids, e.g. proline. Alternatively, a spacer arm may be part of the cross-linking agent, such as in “long-chain SPDP” (Pierce Chem. Co., Rockford, Ill., cat. No. 21651 H). Numerous cross-linking agents, including the ones discussed above, are commercially available. Detailed instructions for their use are readily available from the commercial suppliers. More detailed information on protein cross-linking and conjugate preparation, which is useful in the context of linkage of a further component and the mutated LAIR-1 fragment comprised by the protein according to the present invention comprised by the molecule according to the present invention can be retrieved from: Wong, Chemistry of Protein Conjugation and Cross-Linking, CRC Press (1991).

Cross-linking agents for peptide or protein crosslinking include for example (i) amine-to-amine crosslinkers, e.g. homobifunctional amine-specific protein crosslinking reagents based on NHS-ester and imidoester reactive groups for selective conjugation of primary amines; available in short, long, cleavable, irreversible, membrane permeable, and cell surface varieties; (ii) sulfhydryl-to-carbohydrate crosslinkers, e.g. crosslinking reagents based on maleimide and hydrazide reactive groups for conjugation and formation of covalent crosslinks; (iii) sulfhydryl-to-sulfhydryl crosslinkers, e.g. homobifunctional sulfhydryl-specific crosslinking reagents based on maleimide or pyridyldithiol reactive groups for selective covalent conjugation of protein and peptide thiols (reduced cysteines) to form stable thioether bonds; (iv) photoreactive crosslinkers, e.g. aryl azide, diazirine, and other photo-reactive (light-activated) chemical heterobifunctional crosslinking reagents to conjugate proteins, nucleic acids and other molecular structures involved in receptor-ligand interaction complexes via two-step activation; (v) amine-to-sulfhydryl crosslinkers, e.g. heterobifunctional protein crosslinking reagents for conjugation between primary amine (lysine) and sulfhydryl (cysteine) groups of proteins and other molecules; available with different lengths and types of spacer arms; and (vi) amine-to-amine crosslinkers, e.g. carboxyl-to-amine crosslinkers, e.g. Carbodiimide crosslinking reagents, DCC and EDC (EDAC), for conjugating carboxyl groups (glutamate, aspartate, C-termini) to primary amines (lysine, N-termini) and also N-hydroxysuccinimide (NHS) for stable activation of carboxylates for amine-conjugation.

The linkage between a further component and the mutated LAIR-1 fragment comprised by the protein according to the present invention in the molecule according to the present invention may be directly or indirectly, i.e. the two may directly adjoin or they may be linked by an additional component of the complex, e.g. a spacer or a linker.

A direct linkage may be realized preferably by an amide bridge, if the further component and the mutated LAIR-1 fragment comprised by the protein according to the present invention have reactive amino or carboxy groups. More specifically, if the further component and the mutated LAIR-1 fragment comprised by the protein according to the present invention are peptides, polypeptides or proteins, a peptide bond is preferred. Such a peptide bond may be formed using a chemical synthesis involving both, the further component and the mutated LAIR-1 fragment comprised by the protein according to the present invention (an N-terminal end of one and the C-terminal end of the other) to be linked, or may be formed directly via a protein synthesis of the entire peptide sequence of both, the further component and the mutated LAIR-1 fragment comprised by the protein according to the present invention, wherein both (protein or peptide) are preferably synthesized in one step. Such protein synthesis methods include e.g., without being limited thereto, liquid phase peptide synthesis methods or solid peptide synthesis methods, e.g. solid peptide synthesis methods according to Merrifield, t-Boc solid-phase peptide synthesis, Fmoc solid-phase peptide synthesis, BOP (Benzotriazole-1-yl-oxy-tris-(dimethylamino)-phosphonium hexafluorophosphate) based solid-phase peptide synthesis, etc. Alternatively, ester or ether linkages are preferred.

Moreover, in particular if the further component and the mutated LAIR-1 fragment comprised by the protein according to the present invention are peptides, polypeptides or proteins, a linkage may occur via the side chains, e.g. by a disulfide bridge. Further components of other chemical nature may be likewise attached to the components of peptidic nature, e.g. the mutated LAIR-1 fragment comprised by the protein according to the present invention. The linkage via a side chain will preferably be based on side chain amino, thiol or hydroxyl groups, e.g. via an amide or ester or ether linkage. A linkage of a peptidic main chain with a peptidic side chain of another component may also be via an isopeptide bond. An isopeptide bond is an amide bond that is not present on the main chain of a protein. The bond forms between the carboxyl terminus of one peptide or protein and the amino group of a lysine residue on another (target) peptide or protein.

The molecule according to the present invention may optionally comprise a spacer or linker, which are non-immunologic moieties, which are preferably cleavable, and which may link further component(s) of the molecule to each other and/or to the mutated LAIR-1 fragment comprised by the protein according to the present invention. A linker or spacer may preferably provide further functionalities in addition to linking of the components, and preferably being cleavable, more preferably naturally cleavable inside the target cell, e.g. by enzymatic cleavage. However, such further functionalities do in particular not include any immunological functionalities. Examples of further functionalities, in particular regarding linkers in fusion proteins, can be found in Chen X. et al., 2013: Fusion Protein Linkers: Property, Design and Functionality. Adv Drug Deliv Rev. 65(10): 1357-1369, wherein for example also in vivo cleavable linkers are disclosed. Moreover, Chen X. et al., 2013: Fusion Protein Linkers: Property, Design and Functionality. Adv Drug Deliv Rev. 65(10): 1357-1369 also discloses various linkers, e.g. flexible linkers and rigid linkers, and linker designing tools and databases, which can be useful in the molecule according to the present invention or to design a linker to be used in the molecule according to the present invention.

Said spacer may be peptidic or non-peptidic, preferably the spacer is peptidic. Preferably, a peptidic spacer consists of about 1, 2, 3, 4, 5, 6, 7, 8, 9, or 10 amino acids, more preferably of about 1, 2, 3, 4, or 5 amino acids. The amino acid sequence of the peptidic spacer may be identical to that of the N-terminal or C-terminal flanking region of any of the further component(s) and the mutated LAIR-1 fragment comprised by the protein according to the present invention. Alternatively a peptidic spacer can consist of non-natural amino acid sequences such as an amino acid sequence resulting from conservative amino acid substitutions of said natural flanking regions or sequences of known cleavage sites for proteases such as an enterokinase target site (amino acid sequence: DDDK, SEQ ID NO: 109), factor Xa target site (amino acid sequence: IEDGR, SEQ ID NO: 110), thrombin target site (amino acid sequence: LVPRGS, SEQ ID NO: 111), protease TEV target site (amino acid sequence: ENLYFQG, SEQ ID NO: 112), PreScission protease target site (amino acid sequence LEVLFQGP, SEQ ID NO: 113), polycationic amino acids, e.g. poly K, furin target site (amino acid sequence RX(R/K)R, SEQ ID NO: 114). In a particular embodiment, the peptidic spacer does not contain any Cys (C) residues. In a preferred embodiment the linker sequence contains at least 20%, more preferably at least 40% and even more preferably at least 50% Gly or β-alanine residues, e.g. GlyGlyGlyGlyGly (SEQ ID NO: 115), GlyGlyGlyGly (SEQ ID NO: 116), GGGGS (SEQ ID NO: 117) GlyGlyGly, CysGlyGly or GlyGlyCys, etc. Appropriate linker sequences can be easily selected and prepared by a person skilled in the art. They may be composed of D and/or L amino acids. Further examples of a peptidic spacer include the amino acid sequences EQLE (SEQ ID NO: 118) or TEWT (SEQ ID NO: 119) or any conservative substitutions thereof.

A non-peptidic spacer can include or may be an ester, a thioester, and a di-sulfide.

In particular, the molecule according to the invention may comprise a spacer or linker, in particular a peptidic spacer, placed between the LAIR-1 fragment comprised by the protein according to the present invention and the further component of the molecule according to the present invention.

Fusion Protein According to the Present Invention

Preferably, the protein according to the present invention is a fusion protein, more preferably a recombinant fusion protein. As used herein, a fusion protein is a hybrid protein composed of defined parts of different proteins. Typically, a fusion protein (also referred to as chimeric protein: literally, made of parts from different sources) is created through the joining of two or more genes (or parts of genes) that originally coded for separate proteins. Translation of this fusion gene results in a single or multiple polypeptides with functional properties derived from each of the original proteins.

Recombinant fusion proteins are typically created artificially by recombinant DNA technology. A recombinant fusion protein is a protein created through genetic engineering of a fusion gene. This may involve for example removing the stop codon from a cDNA sequence coding for the first protein, then appending the cDNA sequence of the second protein in frame through ligation or overlap extension PCR. That DNA sequence may then be expressed by a cell as a single protein. The protein may be engineered to include the full sequence of both original proteins, or only a portion of either. If the two entities are proteins, linker (or “spacer”) peptides are preferably added as described above, which make it more likely that the proteins fold independently and behave as expected. For example, the linker may enable protein purification; especially in this case the linker may be engineered with cleavage sites for proteases or chemical agents that enable the liberation of the two separate proteins. This technique may be used for example for identification and purification of proteins, e.g. by fusing a GST protein, FLAG peptide, or a hexa-his peptide (6× His-tag), which can be isolated using affinity chromatography, e.g. with nickel or cobalt resins. Di- or multimeric chimeric proteins may also be manufactured through genetic engineering by fusion to the original proteins of peptide domains that induce artificial protein di- or multimerization (e.g., streptavidin or leucine zippers). Fusion proteins can also be manufactured with toxins or antibodies attached to them.

Naturally occurring antibodies, for example, may be naturally occurring fusion proteins, which are produced by VD) recombination.

Antibodies According to the Present Invention

Preferably, the protein according to the present invention is an antibody, more preferably a monoclonal antibody. In particular, the antibody is an isolated antibody. As used herein, the term “antibody” encompasses various forms of antibodies including, without being limited to, whole antibodies, antibody fragments, human antibodies, chimeric antibodies, humanized antibodies and genetically engineered antibodies (variant or mutant antibodies) as long as the characteristic properties according to the invention are retained. Especially preferred are human or humanized monoclonal antibodies, especially as recombinant human monoclonal antibodies

Human antibodies are well-known in the state of the art (van Dijk, M. A., and van de Winkel, J. G., Curr. Opin. Chem. Biol. 5 (2001) 368-374). Human antibodies can also be produced in transgenic animals (e.g., mice) that are capable, upon immunization, of producing a full repertoire or a selection of human antibodies in the absence of endogenous immunoglobulin production. Transfer of the human germ-line immunoglobulin gene array in such germ-line mutant mice will result in the production of human antibodies upon antigen challenge (see, e.g., Jakobovits, A., et al., Proc. Natl. Acad. Sci. USA 90 (1993) 2551-2555; Jakobovits, A., et al., Nature 362 (1993) 255-258; Bruggemann, M., et al., Year Immunol. 7 (1993) 3340). Human antibodies can also be produced in phage display libraries (Hoogenboom, H. R., and Winter, G., J. Mol. Biol. 227 (1992) 381-388; Marks, J. D., et al., J. Mol. Biol. 222 (1991) 581-597). The techniques of Cole et al. and Boerner et al. are also available for the preparation of human monoclonal antibodies (Cole et al., Monoclonal Antibodies and Cancer Therapy, Alan R. Liss, p. 77 (1985); and Boerner, P., et al., J. Immunol. 147 (1991) 86-95). Preferably, human monoclonal antibodies are prepared by using improved EBV-B cell immortalization as described in Traggiai E, Becker S, Subbarao K, Kolesnikova L, Uematsu Y, Gismondo M R, Murphy B R, Rappuoli R, Lanzavecchia A. (2004): An efficient method to make human monoclonal antibodies from memory B cells: potent neutralization of SARS coronavirus. Nat Med. 10(8):871-5. The term “human antibody” as used herein also comprises such antibodies which are modified, e.g. in the variable region, to generate the properties according to the invention as described herein. As used herein, the term “variable region” (variable region of a light chain (V_{L}), variable region of a heavy chain (V_{H})) denotes each of the pair of light and heavy chains which is involved directly in binding the antibody to the antigen.

Antibodies of the invention can be of any isotype (e.g., IgA, IgG, IgM i.e. an α, γ or μ heavy chain), but will preferably be IgG. Within the IgG isotype, antibodies may be IgG1, IgG2, IgG3 or IgG4 subclass, whereby IgG1 is preferred. Antibodies of the invention may have a κ or a λ light chain.

Preferably, the antibody according to the present invention, or the antigen binding fragment thereof, is a human antibody, a monoclonal antibody, a human monoclonal antibody, a purified antibody, a single chain antibody, Fab, Fab′, F(ab′)2, Fv or scFv.

The antibodies of the invention may thus preferably be human antibodies, monoclonal antibodies, human monoclonal antibodies, recombinant antibodies or purified antibodies. The invention also provides fragments of the antibodies of the invention, particularly fragments that retain the antigen-binding activity of the antibodies. Such fragments include, but are not limited to, single chain antibodies, Fab, Fab′, F(ab′)2, Fv or scFv. Although the specification, including the claims, may, in some places, refer explicitly to antigen binding fragment(s), antibody fragment(s), variant(s) and/or derivative(s) of antibodies, it is understood that the term “antibody” or “antibody of the invention” includes all categories of antibodies, namely, antigen binding fragment(s), antibody fragment(s), variant(s) and derivative(s) of antibodies.

Fragments of the antibodies of the invention can be obtained from the antibodies by methods that include digestion with enzymes, such as pepsin or papain, and/or by cleavage of disulfide bonds by chemical reduction. Alternatively, fragments of the antibodies can be obtained by cloning and expression of part of the sequences of the heavy or light chains. Antibody “fragments” include Fab, Fab′, F(ab′)2 and Fv fragments. The invention also encompasses single-chain Fv fragments (scFv) derived from the heavy and light chains of an antibody of the invention. For example, the invention includes a scFv comprising the CDRs from an antibody of the invention. Also included are heavy or light chain monomers and dimers, single domain heavy chain antibodies, single domain light chain antibodies, as well as single chain antibodies, e.g., single chain Fv in which the heavy and light chain variable domains are joined by a peptide linker.

Antibody fragments of the invention may impart monovalent or multivalent interactions and be contained in a variety of structures as described above. For instance, scFv molecules may be synthesized to create a trivalent “triabody” or a tetravalent “tetrabody.” The scFv molecules may include a domain of the Fc region resulting in bivalent minibodies. In addition, the sequences of the invention may be a component of multispecific molecules in which the sequences of the invention target the epitopes of the invention and other regions of the molecule bind to other targets. Exemplary molecules include, but are not limited to, bispecific Fab2, trispecific Fab3, bispecific scFv, and diabodies (Holliger and Hudson, 2005, Nature Biotechnology 9: 1126-1136).

Antibodies according to the present invention may be provided in purified form. Typically, the antibody will be present in a composition that is substantially free of other polypeptides e.g., where less than 90% (by weight), usually less than 60% and more usually less than 50% of the composition is made up of other polypeptides.

Antibodies according to the present invention may be immunogenic in human and/or in non-human (or heterologous) hosts e.g., in mice. For example, the antibodies may have an idiotope that is immunogenic in non-human hosts, but not in a human host. Antibodies of the invention for human use include those that cannot be easily isolated from hosts such as mice, goats, rabbits, rats, non-primate mammals, etc. and cannot generally be obtained by humanization or from xeno-mice.

In general, the antibody according to the present invention, or the antigen binding fragment thereof, preferably comprises (at least) three CDRs on the heavy chain and (at least) three CDRs on the light chain. In general, complementarity determining regions (CDRs) are the hypervariable regions present in heavy chain variable domains and light chain variable domains. Typically, the CDRs of a heavy chain and the connected light chain of an antibody together form the antigen receptor. Usually, the three CDRs (CDR1, CDR2, and CDR3) are arranged non-consecutively in the variable domain. Since antigen receptors are typically composed of two variable domains (on two different polypeptide chains, i.e. heavy and light chain), there are six CDRs for each antigen receptor (heavy chain: CDRH1, CDRH2, and CDRH3; light chain: CDRL1, CDRL2, and CDRL3). A single antibody molecule usually has two antigen receptors and therefore contains twelve CDRs. The CDRs on the heavy and/or light chain may be separated by framework regions, whereby a framework region (FR) is a region in the variable domain which is less “variable” than the CDR. For example, a chain (or each chain, respectively) may be composed of four framework regions, separated by three CDR.

The sequences of the heavy chains and light chains of several antibodies of the invention, each comprising three CDRs on the heavy chain and three CDRs on the light chain have been determined. The position of the CDR amino acids are defined according to the IMGT numbering system (IMGT: http://www.imgt.org/; cf. Lefranc, M.-P. et al. (2009) Nucleic Acids Res. 37, D1006-D1012). The sequences of the CDRs, heavy chains, light chains as well as the sequences of the nucleic acid molecules encoding the CDRs, heavy chains, light chains of the antibodies of the invention, i.e. of several antibodies according to the invention, are disclosed in the sequence listing. The CDRs of the antibody heavy chains are also referred to as CDRH1, CDRH2 and CDRH3, respectively. Similarly, the CDRs of the antibody light chains are also referred to as CDRL1, CDRL2 and CDRL3, respectively.

Preferably, the antibody according to the present invention comprises a heavy chain comprising CDRH1, CDRH2 and CDRH3 and a light chain comprising CDRL1, CDRL2 and CDRL3, wherein at least one CDR, preferably the heavy chain CDRH3, comprises or consists of a mutated LAIR-1 fragment as described herein. More preferably, the antibody according to the present invention comprises a heavy chain comprising CDRH1, CDRH2 and CDRH3 and a light chain comprising CDRL1, CDRL2 and CDRL3, wherein at least one CDR, preferably the heavy chain CDRH3, comprises or consists of a mutated LAIR-1 fragment according to SEQ ID NO: 10, more preferably according to SEQ ID NO: 15, SEQ ID NO: 16 or SEQ ID NO: 17, even more preferably according to SEQ ID NO: 18, SEQ ID NO: 19 or SEQ ID NO: 20, and particularly preferably according to SEQ ID NO: 21 or SEQ ID NO: 22. Even more preferably, the antibody according to the present invention, or the antigen binding fragment thereof, comprises a heavy chain comprising CDRH1, CDRH2 and CDRH3 and a light chain comprising CDRL1, CDRL2 and CDRL3, wherein at least one CDR, preferably the heavy chain CDRH3, comprises or consists of a mutated LAIR-1 fragment according to any of SEQ ID NOs 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101 and 103 or of a functional sequence variant thereof, preferably the heavy chain CDRH3, comprises or consists of a mutated LAIR-1 fragment according to any of SEQ ID NOs 61, 63, 65, 67, 69, 71, 73, 75, 77, 79, 81, 83, 85, 87, 89, 91, 93, 95, 97, 99, 101 and 103 or of a functional sequence variant thereof. It is also preferred that the antibody according to the present invention, or the antigen binding fragment thereof, comprises a heavy chain comprising CDRH1, CDRH2 and CDRH3 and a light chain comprising CDRL1, CDRL2 and CDRL3, wherein at least one CDR, preferably the heavy chain CDRH3, comprises or consists of a mutated LAIR-1 fragment according to any of SEQ ID NOs 23, 25, 27, 29, 31, 33, 35, 37, 39, 41, 43, 45, 47, 49, 51, 53, 55, 57 and 59 or of a functional sequence variant thereof.

More preferably, the antibody according to the present invention comprises a heavy chain comprising CDRH1, CDRH2 and CDRH3 and a light chain comprising CDRL1, CDRL2 and CDRL3, wherein at least one CDR, preferably the heavy chain CDRH3, comprises an amino acid sequence according to any of SEQ ID NOs: 122, 140, 158, 176, 194, 212, 230, 248, 266, 284, 302, 320, 338, 356, 374, 392, 410, 428, 446, 464, 482, 500, 518, 536, 554 and 572 or a functional sequence variant thereof, preferably according to any of SEQ ID NOs: 320, 392, 464, 500, 536 and 554 or a functional sequence variant thereof, more preferably according to SEQ ID NO: 392 or a functional sequence variant thereof.

Table 3 provides the SEQ ID numbers for the amino acid sequences of the six CDRs of the heavy and light chains, respectively, of exemplary antibodies of the invention.

TABLE 3
SEQ ID Numbers for CDR polypeptides of
exemplary antibodies of the invention.

| | SEQ ID NOs. for CDR Polypeptides | | | | | |
|---|---|---|---|---|---|---|
| | CDRH1 | CDRH2 | CDRH3 | CDRL1 | CDRL2 | CDRL3 |
| MGC1 | 120 | 121 | 122 | 123 | 124/125 | 126 |
| MGC2 | 138 | 139 | 140 | 141 | 142/143 | 144 |
| MGC4 | 156 | 157 | 158 | 159 | 160/161 | 162 |
| MGC5 | 174 | 175 | 176 | 177 | 178/179 | 180 |
| MGC7 | 192 | 193 | 194 | 195 | 196/197 | 198 |
| MGC17 | 210 | 211 | 212 | 213 | 214/215 | 216 |
| MGC26 | 228 | 229 | 230 | 231 | 232/233 | 234 |
| MGC28 | 246 | 247 | 248 | 249 | 250/251 | 252 |
| MGC29 | 264 | 265 | 266 | 267 | 268/269 | 270 |
| MGC32 | 282 | 283 | 284 | 285 | 286/287 | 288 |
| MGC33 | 300 | 301 | 302 | 303 | 304/305 | 306 |
| MGC34 | 318 | 319 | 320 | 321 | 322/323 | 324 |
| MGC35 | 336 | 337 | 338 | 339 | 340/341 | 342 |
| MGC36 | 354 | 355 | 356 | 357 | 358/359 | 360 |
| MGC37 | 372 | 373 | 374 | 375 | 376/377 | 378 |
| MGD21 | 390 | 391 | 392 | 393 | 394/395 | 396 |
| MGD23 | 408 | 409 | 410 | 411 | 412/413 | 414 |
| MGD30 | 426 | 427 | 428 | 429 | 430/431 | 432 |
| MGD33 | 444 | 445 | 446 | 447 | 448/449 | 450 |
| MGD34 | 462 | 463 | 464 | 465 | 466/467 | 468 |
| MGD35 | 480 | 481 | 482 | 483 | 484/485 | 486 |
| MGD39 | 498 | 499 | 500 | 501 | 502/503 | 504 |
| MGD41 | 516 | 517 | 518 | 519 | 520/521 | 522 |
| MGD47 | 534 | 535 | 536 | 537 | 538/539 | 540 |
| MGD55 | 552 | 553 | 554 | 555 | 556/557 | 558 |
| MGD56 | 570 | 571 | 572 | 573 | 574/575 | 576 |

Variant antibodies are also included within the scope of the invention. Thus, variants of the sequences recited in the application are also included within the scope of the invention. Such variants include natural variants generated by somatic mutation in vivo during the immune response or in vitro upon culture of immortalized B cell clones. Alternatively, variants may arise due to the degeneracy of the genetic code or may be produced due to errors in transcription or translation.

Further variants of the antibody sequences having improved affinity and/or potency may be obtained using methods known in the art and are included within the scope of the invention. For example, amino acid substitutions may be used to obtain antibodies with further improved affinity. Alternatively, codon optimization of the nucleotide sequence may be used to improve the efficiency of translation in expression systems for the production of the antibody. Further, polynucleotides comprising a sequence optimized for antibody specificity or neutralizing activity by the application of a directed evolution method to any of the nucleic acid sequences of the invention are also within the scope of the invention.

Preferably, variant antibody sequences may share 70% or more (i.e. 75%, 80%, 85%, 88%, 90%, 92%, 95%, 96%, 97%, 98%, 99% or more) amino acid sequence identity with the sequences recited in the application. Such variants usually have a greater homology to the sequences listed herein in the CDRs of the heavy chain variable region (VH) and light chain variable region (V_{L}) than in the framework region. As is known to one of skill in the art, mutations are more tolerated, i.e., limited or no loss of function (e.g., specificity or neutralization ability) in the framework regions than in the CDRs. The invention thus comprises an antibody, wherein the variation from the sequences provided herein is preferably in the framework region(s) of the antibody or in the nucleic acid residues that encode the framework region(s) of the antibody.

It is also preferred that, the antibody according to the invention comprises a heavy chain CDRH1 with the amino acid sequence of SEQ ID NOs: 120, 138, 156, 174, 192, 210, 228, 246, 264, 282, 300, 318, 336, 354, 372, 390, 408, 426, 444, 462, 480, 498, 516, 534, 552 or 570 or a functional sequence variant thereof; a heavy chain CDRH2 with the amino acid sequence of SEQ ID NOs: 121, 139, 157, 175, 193, 211, 229, 247, 265, 283, 301, 319, 337, 355, 373, 391, 409, 427, 445, 463, 481, 499, 517, 535, 553 or 571 ora functional sequence variant thereof; and a heavy chain CDRH3 with the amino acid sequence of SEQ ID NOs: 122, 140, 158, 176, 194, 212, 230, 248, 266, 284, 302, 320, 338, 356, 374, 392, 410, 428, 446, 464, 482, 500, 518, 536, 554 or 572 ora functional sequence variant thereof. Preferably, an antibody according to the present invention comprises a heavy chain comprising the amino acid sequence of SEQ ID NO: 120 for CDRH1, SEQ ID NO: 121 for CDRH2 and SEQ ID NO: 122 for CDRH3 or functional sequence variants thereof; (ii) SEQ ID NO: 138 for CDRH1, SEQ ID NO: 139 for CDRH2 and SEQ ID NO: 140 for CDRH3 or functional sequence variants thereof; (iii) SEQ ID NO: 156 for CDRH1, SEQ ID NO: 157 for CDRH2 and SEQ ID NO: 158 for CDRH3 or functional sequence variants thereof; (iv) SEQ ID NO: 174 for CDRH1, SEQ ID NO: 175 for CDRH2 and SEQ ID NO: 176 for CDRH3 or functional sequence variants thereof; (v) SEQ ID NO: 192 for CDRH1, SEQ ID NO: 193 for CDRH2 and SEQ ID NO: 194 for CDRH3 or functional sequence variants thereof; (vi) SEQ ID NO: 210 for CDRH1, SEQ ID NO: 211 for CDRH2 and SEQ ID NO: 212 for CDRH3 or functional sequence variants thereof; (vii) SEQ ID NO: 228 for CDRH1, SEQ ID NO: 229 for CDRH2 and SEQ ID NO: 330 for CDRH3 or functional sequence variants thereof; (viii) SEQ ID NO: 246 for CDRH1, SEQ ID NO: 247 for CDRH2 and SEQ ID NO: 248 for CDRH3 or functional sequence variants thereof; (ix) SEQ ID NO: 264 for CDRH1, SEQ ID NO: 265 for CDRH2 and SEQ ID NO: 266 for CDRH3 or functional sequence variants thereof; (x) SEQ ID NO: 282 for CDRH1, SEQ ID NO: 283 for CDRH2 and SEQ ID NO: 284 for CDRH3 or functional sequence variants thereof; (xi) SEQ ID NO: 300 for CDRH1, SEQ ID NO: 301 for CDRH2 and SEQ ID NO: 302 for CDRH3 or functional sequence variants thereof; (xii) SEQ ID NO: 318 for CDRH1, SEQ ID NO: 319 for CDRH2 and SEQ ID NO: 320 for CDRH3 or functional sequence variants thereof; (xiii) SEQ ID NO: 336 for CDRH1, SEQ ID NO: 337 for CDRH2 and SEQ ID NO: 338 for CDRH3 or functional sequence variants thereof; (xiv) SEQ ID NO: 354 for CDRH1, SEQ ID NO: 355 for CDRH2 and SEQ ID NO: 356 for CDRH3 or functional sequence variants thereof; (xv) SEQ ID NO: 372 for CDRH1, SEQ ID NO: 373 for CDRH2 and SEQ ID NO: 374 for CDRH3 or functional sequence variants thereof; (xvi) SEQ ID NO: 390 for CDRH1, SEQ ID NO: 391 for CDRH2 and SEQ ID NO: 392 for CDRH3 or functional sequence variants thereof; (xvii) SEQ ID NO: 408 for CDRH1, SEQ ID NO: 409 for CDRH2 and SEQ ID NO: 410 for CDRH3 or functional sequence variants thereof; (xviii) SEQ ID NO: 426 for CDRH1, SEQ ID NO: 427 for CDRH2 and SEQ ID NO: 428 for CDRH3 or functional sequence variants thereof; (xix) SEQ ID NO: 444 for CDRH1, SEQ ID NO: 445 for CDRH2 and SEQ ID NO: 446 for CDRH3 or functional sequence variants thereof; (xx) SEQ ID NO: 462 for CDRH1, SEQ ID NO: 463 for CDRH2 and SEQ ID NO: 464 for CDRH3 or functional sequence variants thereof; (xxi) SEQ ID NO: 480 for CDRH1, SEQ ID NO: 481 for CDRH2 and SEQ ID NO: 482 for CDRH3 or functional sequence variants thereof; (xxii) SEQ ID NO: 498 for CDRH1, SEQ ID NO: 499 for CDRH2 and SEQ ID NO: 500 for CDRH3 or functional sequence variants thereof; (xxiii) SEQ ID NO: 516 for CDRH1, SEQ ID NO: 517 for CDRH2 and SEQ ID NO: 518 for CDRH3 or functional sequence variants thereof; (xxiv) SEQ ID NO: 534 for CDRH1, SEQ ID NO: 535 for CDRH2 and SEQ ID NO: 536 for CDRH3 or functional sequence variants thereof; (xxv) SEQ ID NO: 552 for CDRH1, SEQ ID NO: 553 for CDRH2 and SEQ ID NO: 554 for CDRH3 or functional sequence variants thereof; or (xxvi) SEQ ID NO: 570 for CDRH1, SEQ ID NO: 571 for CDRH2 and SEQ ID NO: 572 for CDRH3 or functional sequence variants thereof.

More preferably, an antibody according to the present invention comprises a heavy chain comprising the amino acid sequence of (i) SEQ ID NO: 318 for CDRH1, SEQ ID NO: 319 for CDRH2 and SEQ ID NO: 320 for CDRH3 or functional sequence variants thereof; (ii) SEQ ID NO: 390 for CDRH1, SEQ ID NO: 391 for CDRH2 and SEQ ID NO: 392 for CDRH3 or functional sequence variants thereof; (iii) SEQ ID NO: 462 for CDRH1, SEQ ID NO: 463 for CDRH2 and SEQ ID NO: 464 for CDRH3 or functional sequence variants thereof; (iv) SEQ ID NO: 498 for CDRH1, SEQ ID NO: 499 for CDRH2 and SEQ ID NO: 500 for CDRH3 or functional sequence variants thereof; (v) SEQ ID NO: 534 for CDRH1, SEQ ID NO: 535 for CDRH2 and SEQ ID NO: 536 for CDRH3 or functional sequence variants thereof; or (vi) SEQ ID NO: 552 for CDRH1, SEQ ID NO: 553 for CDRH2 and SEQ ID NO: 554 for CDRH3 or functional sequence variants thereof. Even more preferably, an antibody according to the present invention comprises a heavy chain comprising the amino acid sequence of SEQ ID NO: 390 for CDRH1, SEQ ID NO: 391 for CDRH2 and SEQ ID NO: 392 for CDRH3 or functional sequence variants thereof.

Preferably, the isolated antibody or antigen binding fragment according to the present invention comprises a heavy chain variable region having an amino acid sequence that is about 70%, 75%, 80%, 85%, 88%, 90%, 92%, 95%, 96%, 97%, 98%, 99% or 100% identical to the sequence recited in any one of SEQ ID NOs: 134, 152, 170, 188, 206, 224, 242, 260, 278, 296, 314, 332, 350, 368, 386, 404, 422, 440, 458, 476, 494, 512, 530, 548, 566 and 584.

The SEQ ID numbers for the amino acid sequence for the heavy chain variable region (V_{H}) and the light chain variable region (V_{L}) of exemplary antibodies of the invention as well as the SEQ ID numbers for the nucleic acid sequences encoding them are listed below in Table 4.

TABLE 4
SEQ ID Numbers for V_{H} and V_{L} amino acid and nucleic acid residues
for exemplary antibodies according to the present invention.

| | V_{H} amino | V_{L} amino | V_{H} nucleic | V_{L} nucleic |
|---|---|---|---|---|
| | acid | acid | acid | acid |
| MGC1 | 134 | 135 | 136 | 137 |
| MGC2 | 152 | 153 | 154 | 155 |
| MGC4 | 170 | 171 | 172 | 173 |
| MGC5 | 188 | 189 | 190 | 191 |
| MGC7 | 206 | 207 | 208 | 209 |
| MGC17 | 224 | 225 | 226 | 227 |
| MGC26 | 242 | 243 | 244 | 245 |
| MGC28 | 260 | 261 | 262 | 263 |
| MGC29 | 278 | 279 | 280 | 281 |
| MGC32 | 296 | 297 | 298 | 299 |
| MGC33 | 314 | 315 | 316 | 317 |
| MGC34 | 332 | 333 | 334 | 335 |
| MGC35 | 350 | 351 | 352 | 353 |
| MGC36 | 368 | 369 | 370 | 371 |
| MGC37 | 386 | 387 | 388 | 389 |
| MGD21 | 404 | 405 | 406 | 407 |
| MGD23 | 422 | 423 | 424 | 425 |
| MGD30 | 440 | 441 | 442 | 443 |
| MGD33 | 458 | 459 | 460 | 461 |
| MGD34 | 476 | 477 | 478 | 479 |
| MGD35 | 494 | 495 | 496 | 497 |
| MGD39 | 512 | 513 | 514 | 515 |
| MGD41 | 530 | 531 | 532 | 533 |
| MGD47 | 548 | 549 | 550 | 551 |
| MGD55 | 566 | 567 | 568 | 569 |
| MGD56 | 584 | 585 | 586 | 587 |

More preferably, the antibody according to the present invention comprises: (i) a heavy chain variable region comprising the amino acid sequence of SEQ ID NO: 134 or a functional sequence variant thereof and a light chain variable region comprising the amino acid sequence of SEQ ID NO: 135 or a functional sequence variant thereof; or (ii) a heavy chain variable region comprising the amino acid sequence of SEQ ID NO: 152 or a functional sequence variant thereof and a light chain variable region comprising the amino acid sequence of SEQ ID NO: 153 or a functional sequence variant thereof; or (iii) a heavy chain variable region comprising the amino acid sequence of SEQ ID NO: 170 or a functional sequence variant thereof and a light chain variable region comprising the amino acid sequence of SEQ ID NO: 171 or a functional sequence variant thereof; or (iv) a heavy chain variable region comprising the amino acid sequence of SEQ ID NO: 188 or a functional sequence variant thereof and a light chain variable region comprising the amino acid sequence of SEQ ID NO: 189 or a functional sequence variant thereof; or (v) a heavy chain variable region comprising the amino acid sequence of SEQ ID NO: 206 or a functional sequence variant thereof and a light chain variable region comprising the amino acid sequence of SEQ ID NO: 207 or a functional sequence variant thereof; or (vi) a heavy chain variable region comprising the amino acid sequence of SEQ ID NO: 224 or a functional sequence variant thereof and a light chain variable region comprising the amino acid sequence of SEQ ID NO: 225 or a functional sequence variant thereof; or (vii) a heavy chain variable region comprising the amino acid sequence of SEQ ID NO: 242 or a functional sequence variant thereof and a light chain variable region comprising the amino acid sequence of SEQ ID NO: 243 or a functional sequence variant thereof; or (viii) a heavy chain variable region comprising the amino acid sequence of SEQ ID NO: 260 or a functional sequence variant thereof and a light chain variable region comprising the amino acid sequence of SEQ ID NO: 261 or a functional sequence variant thereof; or (ix) a heavy chain variable region comprising the amino acid sequence of SEQ ID NO: 278 or a functional sequence variant thereof and a light chain variable region comprising the amino acid sequence of SEQ ID NO: 279 or a functional sequence variant thereof; or (x) a heavy chain variable region comprising the amino acid sequence of SEQ ID NO: 296 or a functional sequence variant thereof and a light chain variable region comprising the amino acid sequence of SEQ ID NO: 297 or a functional sequence variant thereof; or (xi) a heavy chain variable region comprising the amino acid sequence of SEQ ID NO: 314 or a functional sequence variant thereof and a light chain variable region comprising the amino acid sequence of SEQ ID NO: 315 or a functional sequence variant thereof; or (xii) a heavy chain variable region comprising the amino acid sequence of SEQ ID NO: 332 or a functional sequence variant thereof and a light chain variable region comprising the amino acid sequence of SEQ ID NO: 333 or a functional sequence variant thereof; or (xiii) a heavy chain variable region comprising the amino acid sequence of SEQ ID NO: 350 or a functional sequence variant thereof and a light chain variable region comprising the amino acid sequence of SEQ ID NO: 351 or a functional sequence variant thereof; or (xiv) a heavy chain variable region comprising the amino acid sequence of SEQ ID NO: 368 or a functional sequence variant thereof and a light chain variable region comprising the amino acid sequence of SEQ ID NO: 369 or a functional sequence variant thereof; or (xv) a heavy chain variable region comprising the amino acid sequence of SEQ ID NO: 386 or a functional sequence variant thereof and a light chain variable region comprising the amino acid sequence of SEQ ID NO: 387 or a functional sequence variant thereof; or (xvi) a heavy chain variable region comprising the amino acid sequence of SEQ ID NO: 404 or a functional sequence variant thereof and a light chain variable region comprising the amino acid sequence of SEQ ID NO: 405 or a functional sequence variant thereof; or (xvii) a heavy chain variable region comprising the amino acid sequence of SEQ ID NO: 422 or a functional sequence variant thereof and a light chain variable region comprising the amino acid sequence of SEQ ID NO: 423 or a functional sequence variant thereof; or (xviii) a heavy chain variable region comprising the amino acid sequence of SEQ ID NO: 440 or a functional sequence variant thereof and a light chain variable region comprising the amino acid sequence of SEQ ID NO: 441 or a functional sequence variant thereof; or (xix) a heavy chain variable region comprising the amino acid sequence of SEQ ID NO: 458 or a functional sequence variant thereof and a light chain variable region comprising the amino acid sequence of SEQ ID NO: 459 or a functional sequence variant thereof; or (xx) a heavy chain variable region comprising the amino acid sequence of SEQ ID NO: 476 or a functional sequence variant thereof and a light chain variable region comprising the amino acid sequence of SEQ ID NO: 477 or a functional sequence variant thereof; or (xxi) a heavy chain variable region comprising the amino acid sequence of SEQ ID NO: 494 or a functional sequence variant thereof and a light chain variable region comprising the amino acid sequence of SEQ ID NO: 495 or a functional sequence variant thereof; or (xxii) a heavy chain variable region comprising the amino acid sequence of SEQ ID NO: 512 or a functional sequence variant thereof and a light chain variable region comprising the amino acid sequence of SEQ ID NO: 513 or a functional sequence variant thereof; or (xxiii) a heavy chain variable region comprising the amino acid sequence of SEQ ID NO: 530 or a functional sequence variant thereof and a light chain variable region comprising the amino acid sequence of SEQ ID NO: 531 or a functional sequence variant thereof; or (xxiv) a heavy chain variable region comprising the amino acid sequence of SEQ ID NO: 548 or a functional sequence variant thereof and a light chain variable region comprising the amino acid sequence of SEQ ID NO: 549 or a functional sequence variant thereof; or (xxv) a heavy chain variable region comprising the amino acid sequence of SEQ ID NO: 566 or a functional sequence variant thereof and a light chain variable region comprising the amino acid sequence of SEQ ID NO: 567 or a functional sequence variant thereof; or (xxvi) a heavy chain variable region comprising the amino acid sequence of SEQ ID NO: 584 or a functional sequence variant thereof and a light chain variable region comprising the amino acid sequence of SEQ ID NO: 585 or a functional sequence variant thereof.

Even more preferably, the antibody according to the present invention comprises: (i) a heavy chain variable region comprising the amino acid sequence of SEQ ID NO: 332 or a functional sequence variant thereof and a light chain variable region comprising the amino acid sequence of SEQ ID NO: 333 or a functional sequence variant thereof; or (ii) a heavy chain variable region comprising the amino acid sequence of SEQ ID NO: 404 or a functional sequence variant thereof and a light chain variable region comprising the amino acid sequence of SEQ ID NO: 405 or a functional sequence variant thereof; or (iii) a heavy chain variable region comprising the amino acid sequence of SEQ ID NO: 476 or a functional sequence variant thereof and a light chain variable region comprising the amino acid sequence of SEQ ID NO: 477 or a functional sequence variant thereof; or (iv) a heavy chain variable region comprising the amino acid sequence of SEQ ID NO: 512 or a functional sequence variant thereof and a light chain variable region comprising the amino acid sequence of SEQ ID NO: 513 or a functional sequence variant thereof; or (v) a heavy chain variable region comprising the amino acid sequence of SEQ ID NO: 548 or a functional sequence variant thereof and a light chain variable region comprising the amino acid sequence of SEQ ID NO: 549 or a functional sequence variant thereof; or (vi) a heavy chain variable region comprising the amino acid sequence of SEQ ID NO: 566 or a functional sequence variant thereof and a light chain variable region comprising the amino acid sequence of SEQ ID NO: 567 or a functional sequence variant thereof. Particularly preferably, an antibody according to the present invention comprises a heavy chain variable region comprising the amino acid sequence of SEQ ID NO: 404 or a functional sequence variant thereof and a light chain variable region comprising the amino acid sequence of SEQ ID NO: 405 or a functional sequence variant thereof.

Particularly preferred examples of antibodies according to the present invention are shown below, in Table 5. The CDR sequences as well as the sequences of the heavy and light chain variable region are shown for each exemplary antibody separately, while the sequences for the constant regions, which are identical for all exemplary antibodies, are shown only once subsequently (cf. SEQ ID NOs 588-593 for constant regions).

TABLE 5
Sequences and SEQ ID Numbers of preferred exemplary antibodies
according to the present invention.

| | | |
|---|---|---|
| SEQ | | |
| ID | | |
| NO | Description | Sequence* |

MGC1 ANTIBODY

| | | |
|---|---|---|
| 120 | CDRH1 aa | GFNFRKSW |
| 121 | CDRH2 aa | IREDGSES |
| 122 | CDRH3 aa | ARDRFCNDDEIHRHGQEDLPRPSISAAEGTVIPLGSHVTFVCR |
| | | GPVGVQTFRLEKDSRSIYNDTENVSQPSPSESEARFRIDSVSEG |
| | | NAGLYRCVYYKAPKWSAQSDYLELLVKGQEVTWALFTSCGG |
| | | DGEEPDYDMDV |
| 123 | CDRL1 aa | QSVLYRSKNKNY |
| 124 | CDRL2 aa | STS |
| 125 | CDRL2 long aa | YYCLQYYITPYTFGQ |
| 126 | CDRL3 aa | LQYYITPYT |
| 127 | CDRH1 nuc | gggttcaactttagaaagtcttgg |
| 128 | CDRH2 nuc | ataagagaagatggaagtgagagt |
| 129 | CDRH3 nuc | gcgagagatagattctgcaatgatgatgagattcacagacacggacaagaagatctgc |
| | | ccagaccctccatctcggctgccgaaggcaccgtgatccccctggggagccatgtga |
| | | ctttcgtgtgccggggcccggttggggttcaaacattccgcctggagaaggacagtaga |
| | | tccatatacaatgatactgaaaatgtgtctcaacctagtccatctgagtcagaggccaga |
| | | tttcgcattgactcagtgagtgaaggaaatgccggactttatcggtgcgtctattataagg |
| | | cccctaaatggtctgcgcagagtgattacctggagctgctggtgaaaggtcaggaagtc |
| | | acctgggccctgtttacctcctgtggtggtgatggagaggaacccgactacgacatgga |
| | | cgtc |
| 130 | CDRL1 nuc | cagagtgtatacaggtccaagaataagaactac |
| 131 | CDRL2 nuc | tcgacatct |
| 132 | CDRL2 long nuc | ctcatttactcgacatctactcgggcg |
| 133 | CDRL3 nuc | ctgcaatattatattactccctacact |
| 134 | heavy chain aa | EVQLVESGGGLVQPGGSLRLSCVASGFNFRKSWMGWVRQA |
| | | PGKGLEWVANIREDGSESFYADSVKGRFTVSRDNAKKSLYLHI |
| | | NSLRAEDTAVYYCARDRFCNDDEIHRHGQEDLPRPSISAAEG |
| | | TVIPLGSHVTFVCRGPVGVQTFRLEKDSRSIYNDTENVSQPSPS |
| | | ESEARFRIDSVSEGNAGLYRCVYYKAPKWSAQSDYLELLVKGQ |
| | | EVTWALFTSCGGDGEEPDYDMDVRGKGTTVTVSS |
| 135 | light chain aa | DIVMTQSPDSLAVSLGERATINCKSSQSVLYRSKNKNYLAWF |
| | | QQKPGQPPKVLIYSTSTRASGVPDRFTGSGSGTDFTLTISSLQA |
| | | EDVAVYYCLQYYITPYTFGQGTKLEIK |
| 136 | heavy chain nuc | gaggtgcagctggtggagtctgggggaggcttggtccagccgggggggtccctgaga |
| | | ctctcctgtgtagcctctgggttcaactttagaaagtcttggatgggttgggtccgccagg |
| | | ctccagggaaggggctggagtgggtggcaaacataagagaagatggaagtgagagtt |
| | | tctatgcggactctgtgaagggccgcttcaccgtctccagagacaacgccaagaaatc |
| | | actgtatctccatatcaacagcctgagagccgaggacacggctgtctattactgtgcga |
| | | gagatagattctgcaatgatgatgagattcacagacacggacaagaagatctgcccag |
| | | accctccatctcggctgccgaaggcaccgtgatccccctggggagccatgtgactttc |
| | | gtgtgccggggcccggttggggttcaaacattccgcctggagaaggacagtagatcca |
| | | tatacaatgatactgaaaatgtgtctcaacctagtccatctgagtcagaggccagatttc |
| | | gcattgactcagtgagtgaaggaaatgccggactttatcggtgcgtctattataaggccc |
| | | ctaaatggtctgcgcagagtgattacctggagctgctggtgaaaggtcaggaagtcacc |
| | | tgggccctgtttacctcctgtggtggtgatggagaggaacccgactacgacatggacgt |
| | | ccggggcaaagggaccacggtcaccgtctcctca |
| 137 | light chain nuc | gacatcgtgatgacccagtctccagactccctggctgtgtctctgggcgagagggcca |
| | | ccatcaactgcaagtccagtcagagtgttttatacaggtccaagaataagaactacttag |
| | | cttggttccagcagaaaccaggacagcctcctaaggtgctcatttactcgacatctactc |
| | | gggcgtccggggtccctgaccgattcactggcagcgggtctgggacagatttcactctc |
| | | accatcagcagcctgcaggctgaagatgtggcagtttattactgtctgcaatattatatta |
| | | ctccctacacttttggccaggggaccaagttggagatcaaa |

MGC2 ANTIBODY

| | | |
|---|---|---|
| 138 | CDRH1 aa | GFTFSNFW |
| 139 | CDRH2 aa | IKEDGSEK |
| 140 | CDRH3 aa | VRERFCSNHIHKEEHLPRPSISPEPGTVITLGSHVTFVCRGPVGV |
| | | QTFRLEKDSRSTYNDTEDVSQPSPSESEARFRIDSVSEGYAGLY |
| | | RCLYYKPPKWSEQSDYLELLVKGDDVTWALYPSCGGDGEAS |
| | | DYNMDV |
| 141 | CDRL1 aa | QRFSGW |
| 142 | CDRL2 aa | KAS |
| 143 | CDRL2 long aa | LIYKASPLA |
| 144 | CDRL3 aa | QHYSNYSYT |
| 145 | CDRH1 nuc | ggattcacctttagtaacttttgg |
| 146 | CDRH2 nuc | ataaaggaagatggaagtgagaaa |
| 147 | CDRH3 nuc | gtgagagagagattctgcagtaatcatatccacaaagaagagcatctgcccagaccct |
| | | ccatctcgcctgagccaggcaccgtgatcaccctggggagccatgtgactttcgtgtgc |
| | | cggggcccggttggggttcaaacattccgcctggagaaggacagtagatccacatac |
| | | aatgatactgaagatgtgtctcaacctagtccatctgagtcagaggccagattccgcatt |
| | | gactcagtaagtgaaggatatgccgggattatcgctgcctctattataagccccctaaa |
| | | tggtctgagcagagtgactacctggagctgctggtgaaaggtgacgacgtcacctggg |
| | | ccctgtacccctcttgtggtggtgatggagaggcttccgactacaacatggacgtc |
| 148 | CDRL1 nuc | cagcgttttagtggctgg |
| 149 | CDRL2 nuc | aaggcgtct |
| 150 | CDRL2 long nuc | ctgatctataaggcgtctcctttagca |
| 151 | CDRL3 nuc | caacactacagtaattattcatatact |
| 152 | heavy chain aa | EVQLVESGGGLVQPGGSLRLSCAASGFTFSNFWMGWVRQT |
| | | PGKGLEWVANIKEDGSEKYYVDSVRGRFTISRDSAKNSLYLQ |
| | | MNSLRAEDTAVYYCVRERFCSNHIHKEENLPRPSISPEPGTVITL |
| | | GSHVTFVCRGPVGVQTFRLEKDSRSTYNDTEDVSQPSPSESEA |
| | | RFRIDSVSEGYAGLYRCLYYKPPWSEQSDYLELLVKGDDVT |
| | | WALYPSCGGDGEASDYNMDVWGKGTTVTVSS |
| 153 | light chain aa | DIQMTQSPSTLSASVGDRVTISCRASQRFSGWLAWYQQKPG |
| | | KAPNLLIYKASPLAGGGPSRFSGSGSGTDFTLTISSLQPDDSAT |
| | | YYCQHYSNYSYTFGQGTKLEIR |
| 154 | heavy chain nuc | gaggtgcagctggtggagtctgggggaggcttggtccagcctggggggtccctgagac |
| | | tctcctgtgcagcctctggattcacctttagtaacttttggatgggttgggtccgccagact |
| | | ccagggaaggggctggagtgggtggccaatataaaggaagatggaagtgagaaata |
| | | ctatgtggactctgtgaggggccgattcaccatctccagagacagcgccaagaactca |
| | | ctttatctgcagatgaacagcctgagagccgaggacacggctgtctattattgtgtgaga |
| | | gagagattctgcagtaatcatatccacaaagaagagcatctgcccagaccctccatct |
| | | cgcctgagccaggcaccgtgatcaccctggggagccatgtgactttcgtgtgccgggg |
| | | cccggttggggttcaaacattccgcctggagaaggacagtagatccacatacaatgat |
| | | actgaagatgtgtctcaacctagtccatctgagtcagaggccagattccgcattgactca |
| | | gtaagtgaaggatatgccgggctttatcgctgcctctattataagccccctaaatggtctg |
| | | agcagagtgactacctggagctgctggtgaaaggtgacgacgtcacctgggccctgta |
| | | cccctcttgtggtggtgatggagaggcttccgactacaacatggacgtctggggcaaag |
| | | ggaccacggtcaccgtctcctca |
| 155 | light chain nuc | gacatccagatgacccagtctccttccaccctgtctgcatctgtgggagacagagtcac |
| | | catctcttgccgggccagtcagcgttttagtggctggttggcctggtatcagcagaaacc |
| | | agggaaagcccctaacctcctgatctataaggcgtctcctttagcaggtgggggcccat |
| | | caaggttcagcggcagtggatctgggacagacttcactctcaccatcagcagcctgca |
| | | gcctgatgattctgcaacttattactgccaacactacagtaattattcatatacttttggcca |
| | | ggggaccaagctggagatcaga |

MGC4 ANTIBODY

| | | |
|---|---|---|
| 156 | CDRH1 aa | GFNSRSYW |
| 157 | CDRH2 aa | INQDGTEK |
| 158 | CDRH3 aa | ARDRFCGGESHLHGEEDLPRPSISAEPDTVIPLGSHVTFVCRGP |
| | | VGVHTFRLERGWRYNDTEDVSQAGPSESEARFRIDSVREGNA |
| | | GLYRCIYYIAPKWSEQSDYLELRVKGGDVTWALLTYCGGDGE |
| | | ESDYPMDV |
| 159 | CDRL1 aa | TGPVTSAYY |
| 160 | CDRL2 aa | SIN |
| 161 | CDRL2 long aa | LIYSINKKH |
| 162 | CDRL3 aa | LLSCGGAQPWV |
| 163 | CDRH1 nuc | ggattcaactctcgtagttattgg |
| 164 | CDRH2 nuc | ataaatcaagatgggactgagaaa |
| 165 | CDRH3 nuc | gcgagagacagattctgtggtggtgagagtcacttgcacggagaagaagatctgccca |
| | | gaccctccatctcggctgagccagacaccgtaatccccctggggagccatgtgacttt |
| | | cgtgtgccggggcccggttggggttcacacattccgcctggagagggggtggaggtac |
| | | aacgacactgaagatgtgtctcaagctggtccatctgagtcagaggccagattccgcat |
| | | tgactcggtaagggaaggaaatgccgggctttatcgatgcatctattacatagcccctaa |
| | | atggtctgagcagagtgactacctggagctgcgggtgaaaggtggggacgtcacctgg |
| | | gccctgttaacgtactgtggcggtgatggagaggaatccgactaccccatggacgtc |
| 166 | CDRL1 nuc | actggacctgtcaccagtgcttactat |
| 167 | CDRL2 nuc | agtataaac |
| 168 | CDRL2 long nuc | cttatttatagtataaacaaaaaacac |
| 169 | CDRL3 nuc | ctgctctcctgtggtggtgctcagccttgggtg |
| 170 | heavy chain aa | EVQLVESGGGLVQPGGSLRLSCEGSGFNSRSYWMTWVRQA |
| | | PGKGLEWVASINQDGTEKNYVDSVKGRFTISRDSAKNSLYLQ |
| | | MSSLRADDTAVYYCARDRFCGGESHLHGEEDLPRPSISAEPDT |
| | | VIPLGSHVTFVCRGPVGVHTFRLERGWRYNDTEDVSQAGPSE |
| | | SEARFRIDSVREGNAGLYRCIYYIAPKWSEQSDYLELRVKGGD |
| | | VTWALLTYCGGDGEESDYPMDVWGKGTTVTVSS |
| 171 | light chain aa | QTVVTQEPSLTVSPGGTVTLTCASSTGPVTSAYYPNWFQQKP |
| | | GQAPRSLIYSINKKHSWTPARFSGSLLGGKAALTLSGVQPEDE |
| | | ADYYCLLSCGGAQPWVFGGGTKLTVQ |
| 172 | heavy chain nuc | gaggtgcagctggtggagtctgggggaggcttggtacagcctggggggtccctgagac |
| | | tctcctgtgaaggctctggattcaactctcgtagttattggatgacctgggtccgccaggc |
| | | tccagggaaggggctggagtgggtggccagtataaatcaagatgggactgagaaaaa |
| | | ttatgtggactctgtgaagggccggttcaccatctccagagactccgccaagaactcac |
| | | tgtatctgcaaatgagcagcctgagagccgacgacacggctgtatattactgtgcgaga |
| | | gacagattctgtggtggtgagagtcacttgcacggagaagaagatctgcccagaccct |
| | | ccatctcggctgagccagacaccgtaatccccctggggagccatgtgactttcgtgtgc |
| | | cggggcccggaggggttcacacattccgcaggagagggggtggaggtacaacgac |
| | | actgaagatgtgtctcaagctggtccatctgagtcagaggccagattccgcattgactcg |
| | | gtaagggaaggaaatgccgggctttatcgatgcatctattacatagcccctaaatggtct |
| | | gagcagagtgactacctggagctgcgggtgaaaggtggggacgtcacctgggccctg |
| | | ttaacgtactgtggcggtgatggagaggaatccgactaccccatggacgtctggggca |
| | | aagggaccacggtcaccgtctcctca |
| 173 | light chain nuc | cagactgtggttactcaggagccctcactgactgtgtccccaggagggacagtcactct |
| | | cacctgtgcttccagcactggacctgtcaccagtgcttactatccaaactggttccagca |
| | | gaagcctggacaagcacccaggtctcttatttatagtataaacaaaaaacactcctgga |
| | | cccctgcccggttctcaggctccctccttgggggcaaagctgccctgacactgtcaggt |
| | | gtacagcctgaggacgaggctgactattactgcctgctctcctgtggtggtgctcagcct |
| | | tgggtgttcggcggagggaccaagctgaccgtccaag |

MGC5 ANTIBODY

| | | |
|---|---|---|
| 174 | CDRH1 aa | GFNSRSYW |
| 175 | CDRH2 aa | INQDGTEK |
| 176 | CDRH3 aa | ARDRFCGGESHLHGEEDLPRPSISAEPGTVIPLGSHVTFVCRGP |
| | | VGVHTFRLERGWRYNDTEDVSQAGPSQSEARFRIDSVREGN |
| | | AGLYRCLYYIPPKWSEQSDYLELRVKGGDVTWALLTYCGGD |
| | | GEESDYPMDV |
| 177 | CDRL1 aa | TGPVTSAYY |
| 178 | CDRL2 aa | NIN |
| 179 | CDRL2 long aa | LIYNINKKH |
| 180 | CDRL3 aa | LLSCGGAQPWV |
| 181 | CDRH1 nuc | ggattcaactctcgtagttattgg |
| 182 | CDRH2 nuc | ataaatcaagatggaactgagaaa |
| 183 | CDRH3 nuc | gcgagagacagattctgtggtggtgagagtcacttgcacggagaagaagatctgccca |
| | | gaccctccatctcggctgagccaggcaccgtgatccccctggggagccatgtgacttt |
| | | cgtgtgccggggccccgttggggttcacacattccgcctggagagggggtggagatac |
| | | aacgacactgaagatgtgtctcaagctggtccatctcagtcagaggccagattccgcat |
| | | tgactcggtaagggaaggaaatgccgggctttatcgatgcctctattacataccccctaa |
| | | atggtctgagcagagtgactacctggaactgcgggtgaaaggtggggacgtcacctgg |
| | | gccctgttaacgtactgtggtggtgatggagaggaatccgactaccccatggacgtc |
| 184 | CDRL1 nuc | actggacctgtcaccagtgcttactat |
| 185 | CDRL2 nuc | aatataaac |
| 186 | CDRL2 long nuc | cttatttataatataaacaaaaaacac |
| 187 | CDRL3 nuc | ctgctctcctgtggtggtgctcagccttgggtg |
| 188 | heavy chain aa | EVQLVESGGGLVQPGGSLRLSCEASGFNSRSYWMTWVRQAP |
| | | GKGLEWVATINQDGTEKNYVDSVRGRFTISRDTAKNSLFLQ |
| | | MNSLRAEDTAVYYCARDRFCGGESHLHGEEDLPRPSISAEPGT |
| | | VIPLGSHVTFVCRGPVGVHTFRLERGWRYNDTEDVSQAGPS |
| | | QSEARFRIDSVREGNAGLYRCLYYIPPKWSEQSDYLELRVKGG |
| | | DVTWALLTYCGGDGEESDYPMDVWGKGTTVTVSS |
| 189 | light chain aa | QTVVTQEPSLTVSPGGTVTLTCASNTGPVTSAYYPNWFQQKP |
| | | GQAPRSLIYNINKKHSWTPARFSGSLLGGKAALTLSGVQPEDE |
| | | ADYYCLLSCGGAQPWVFGGGTKLTVQ |
| 190 | heavy chain nuc | gaggtgcagctggtggagtctgggggaggcaggtccagcctggggggtcactgagac |
| | | tctcctgtgaagcctctggattcaactctcgtagttattggatgacctgggtccgccaggc |
| | | tccagggaaggggctggagtgggtggccactataaatcaagatggaactgagaaaaa |
| | | ttatgtggactctgtgaggggccggttcaccatctccagagacaccgccaagaactca |
| | | ctgtttctgcaaatgaacagcctgagagccgaggacacggctgtatattactgcgcgag |
| | | agacagattctgtggtggtgagagtcacttgcacggagaagaagatctgcccagaccc |
| | | tccatctcggctgagccaggcaccgtgatccccctggggagccatgtgactacgtgtg |
| | | ccggggccccgttggggttcacacattccgcctggagagggggtggagatacaacga |
| | | cactgaagatgtgtctcaagctggtccatctcagtcagaggccagattccgcattgactc |
| | | ggtaagggaaggaaatgccgggctttatcgatgcctctattacataccccctaaatggtc |
| | | tgagcagagtgactacctggaactgcgggtgaaaggtggggacgtcacctgggccct |
| | | gttaacgtactgtggtggtgatggagaggaatccgactaccccatggacgtctggggca |
| | | aagggaccacggtcaccgtctcctca |
| 191 | light chain nuc | cagactgtggtgactcaggagccctcactgactgtgtccccaggagggacagtcactc |
| | | tcacctgtgcttccaacactggacctgtcaccagtgcttactatccaaactggaccagc |
| | | agaagcctggacaagcacccaggtctcttatttataatataaacaaaaaacactcctgg |
| | | acccctgcccggttctcaggctccctccttgggggcaaagctgccctgacactgtcag |
| | | gtgtacagcctgaggacgaggctgactattactgcctgctctcctgtggtggtgctcagc |
| | | cttgggtgttcggcggagggaccaagctgaccgtccaa |

MGC7 ANTIBODY

| | | |
|---|---|---|
| 192 | CDRH1 aa | GFTFRNYW |
| 193 | CDRH2 aa | IRQDGSEK |
| 194 | CDRH3 aa | VRDKFCSDENHMHVADDLPRPSISPEPGTVIPLGSHVTFVCRG |
| | | PVGVQTFRLEKDRRSTYNDTEDVSQPSPSESEARFRIDSVTEGN |
| | | AGLYRCVYYKPPKWSDQSDFLELLVKGEDVTWALFPHCGAD |
| | | GEDSDYYMDV |
| 195 | CDRL1 aa | QGLSTW |
| 196 | CDRL2 aa | AAS |
| 197 | CDRL2 long aa | LIYAASSLQ |
| 198 | CDRL3 aa | QQANSFPLT |
| 199 | CDRH1 nuc | ggattcaccttcagaaattattgg |
| 200 | CDRH2 nuc | ataaggcaagatggaagtgagaag |
| 201 | CDRH3 nuc | gtgagagataaattctgcagtgatgagaatcacatgcacgtagcagatgatctgcccag |
| | | accctctatctcgcctgagccaggcaccgtgatccccctggggagccatgtgactttcg |
| | | tgtgtcggggcccggttggggttcaaacattccgcctggagaaggacagaagatccac |
| | | atacaatgatactgaagatgtgtctcaacctagtccatctgagtcagaggccagattccg |
| | | cattgactcagtaactgaaggaaatgccgggctttatcgctgcgtctattataagccccc |
| | | taaatggtctgaccagagtgacttcctggagttgctggtgaagggtgaggacgtcacctg |
| | | ggccctgttcccccattgtggtgctgatggagaggactccgactactacatggacgtc |
| 202 | CDRL1 nuc | cagggtcttagtacctgg |
| 203 | CDRL2 nuc | gctgcatcc |
| 204 | CDRL2 long nuc | tattattgtcaacaggctaacagtttccctctcactttcggcgga |
| 205 | CDRL3 nuc | caacaggctaacagtttccctctcact |
| 206 | heavy chain aa | EVQLVESGGDLVQPGGSLRLSCAASGFTFRNYWMSWVRQTP |
| | | GKGLEWVANIRQDGSEKYYVDSVKGRFTISRDNAKNLLYLQ |
| | | MNSLRAEDTAVYYCVRDKFCSDENHMHVADDLPRPSISPEPG |
| | | TVIPLGSHVTFVCRGPVGVQTFRLEKDRRSTYNDTEDVSQPSP |
| | | SESEARFRIDSVTEGNAGLYRCVYYKPPKWSDQSDFLELLVKG |
| | | EDVTWALFPHCGADGEDSDYYMDVWGKGTTVTVSS |
| 207 | light chain aa | DIQMTQSPSSVSASVGDRVTITCRASQGLSTWLAWYQQKPG |
| | | KAPKILIYAASSLQSGVPSRFSGSGSGTDFTLTISSLQPEDFATYY |
| | | CQQANSFPLTFGGGTKVEIK |
| 208 | heavy chain nuc | gaggtgcagctggtggagtctgggggagacttggtccagcctggggggtccctgagac |
| | | tctcctgtgcagcctctggattcaccttcagaaattattggatgagttgggtccgccagac |
| | | tccagggaagggactggagtgggtggccaacataaggcaagatggaagtgagaagt |
| | | attatgtggactctgtgaagggccgattcaccatctccagagacaacgccaagaactta |
| | | ttatatctacaaatgaacagcctgagagccgaggacacggctgtgtattactgtgtgaga |
| | | gataaattctgcagtgatgagaatcacatgcacgtagcagatgatctgcccagaccctc |
| | | tatctcgcctgagccaggcaccgtgatccccctggggagccatgtgactttcgtgtgtcg |
| | | gggcccggttggggttcaaacattccgcctggagaaggacagaagatccacatacaa |
| | | tgatactgaagatgtgtctcaacctagtccatctgagtcagaggccagattccgcattga |
| | | ctcagtaactgaaggaaatgccgggctttatcgctgcgtctattataagccccctaaatg |
| | | gtctgaccagagtgacttcctggagttgctggtgaagggtgaggacgtcacctgggccc |
| | | tgttcccccattgtggtgctgatggagaggactccgactactacatggacgtctggggca |
| | | aagggaccacggtcaccgtctcctca |
| 209 | light chain nuc | gacatccagatgacccagtctccatcttccgtgtctgcatctgtaggagacagagtcac |
| | | catcacttgtcgggcgagtcagggtcttagtacctggttagcctggtatcagcagaaacc |
| | | agggaaagcccctaagatcctgatctatgctgcatccagtttgcaaagtggggtcccat |
| | | caaggttcagcggcagtggatctgggacagatttcactctcaccatcagcagcctgca |
| | | gcctgaagattttgcaacttattattgtcaacaggctaacagtttccctctcactttcggcg |
| | | gagggaccaaggtggagatcaaa |

MGC17 ANTIBODY

| | | |
|---|---|---|
| 210 | CDRH1 aa | GFNFRKSW |
| 211 | CDRH2 aa | IREDGSKA |
| 212 | CDRH3 aa | ARDRFCSDDEDHSHGAEDLPRPSISAEEGTVIPLGSRLTFVCRG |
| | | PVGVHTFRLERDRRSTYNDTEDVSHPSPSESEARFRIDSVSEGN |
| | | AGLYRCVYYKSPEWSKQSDYLELLVKGQEVTWALFTSCGGD |
| | | GEVPDYDMDV |
| 213 | CDRL1 aa | QSVLYRSKNKKY |
| 214 | CDRL2 aa | WTS |
| 215 | CDRL2 long aa | LIYWTSTRA |
| 216 | CDRL3 aa | QQYFIFPYT |
| 217 | CDRH1 nuc | gggttcaattttagaaagtcttgg |
| 218 | CDRH2 nuc | ataagagaagatggaagtaaggca |
| 219 | CDRH3 nuc | gcgagagatagattctgcagtgatgatgaggatcacagccacggagcagaagatctg |
| | | cccagaccctccatctcggctgaggaaggcaccgtgattcccctggggagccgtctg |
| | | actttcgtgtgccggggcccggttggggttcacacattccgcctggagagggaccgtag |
| | | atccacatacaatgatactgaagatgtgtctcaccctagtccatctgagtctgaggccag |
| | | atttcgcattgactcagtgagtgaaggaaatgccgggctttatcgctgcgtctattataagt |
| | | cccctgaatggtctaagcagagtgattacctggagctgctggtgaaaggtcaggaagtc |
| | | acctgggccctgtttacttcttgtggtggtgatggagaggtacccgactacgacatggac |
| | | gtc |
| 220 | CDRL1 nuc | cagagtgttttatacaggtccaagaataagaaatat |
| 221 | CDRL2 nuc | tggacatct |
| 222 | CDRL2 long nuc | ctcatttactggacatctactcgggcg |
| 223 | CDRL3 nuc | cagcagtattttatttttccgtacact |
| 224 | heavy chain aa | EVQLVESGGGLVQPGGSLKLSCVASGFNFRKSWMSWVRQA |
| | | PGKGLEWVANIREDGSKAYYVDSVKGRFTVSRDNAKNSLYL |
| | | QINSLRADDTAVYYCARDRFCSDDEDHSHGAEDLPRPSISAEE |
| | | GTVIPLGSRLTFVCRGPVGVHTFRLERDRRSTYNDTEDVSHPS |
| | | PSESEARFRIDSVSEGNAGLYRCVYYKSPEWSKQSDYLELLVKG |
| | | QEVTWALFTSCGGDGEVPDYDMDVRGKGTTVTVSS |
| 225 | light chain aa | DIVMTQSPDSLAVSLGERATINCKSSQSVLYRSKNKKYLAWFQ |
| | | QRPGQPPKVLIYWTSTRASGVPDRFSGSGSGTDFTLTISSLQA |
| | | DDVAVYYCQQYFIFPYTFGQGTKLEIR |
| 226 | heavy chain nuc | gaggtgcagctggtggagtcggggggaggcttggtccagcctggggggtccctgaaa |
| | | ctgtcctgtgtagcctctgggttcaattttagaaagtcttggatgagttgggtccgccaggc |
| | | tccagggaaggggctggagtgggtggcaaacataagagaagatggaagtaaggcat |
| | | actatgtggactctgtcaagggccgattcaccgtctccagagacaacgccaagaactc |
| | | gctgtatctgcagatcaacagcctgagagccgacgacacggctgtctattactgtgcga |
| | | gagatagattctgcagtgatgatgaggatcacagccacggagcagaagatctgccca |
| | | gaccctccatctcggctgaggaaggcaccgtgattcccctggggagccgtctgactttc |
| | | gtgtgccggggcccggttggggttcacacattccgcctggagagggaccgtagatcca |
| | | catacaatgatactgaagatgtgtctcaccctagtccatctgagtctgaggccagatttc |
| | | gcattgactcagtgagtgaaggaaatgccgggctttatcgctgcgtctattataagtccc |
| | | ctgaatggtctaagcagagtgattacctggagctgctggtgaaaggtcaggaagtcacc |
| | | tgggccctgtttacttcttgtggtggtgatggagaggtacccgactacgacatggacgtc |
| | | cggggcaaagggaccacggtcaccgtctcttca |
| 227 | light chain nuc | gacatcgtgatgacccaatctcctgactccctggctgtgtctctgggcgagagggccac |
| | | catcaactgcaagtccagccagagtgttttatacaggtccaagaataagaaatatttagc |
| | | ttggttccagcagagaccaggacagcctcctaaggttctcatttactggacatctactcg |
| | | ggcgtccggggtccctgaccgattcagtggcagcgggtctgggacagatttcactctca |
| | | ccatcagcagcctgcaggctgatgatgtggcagtttattattgtcagcagtattttatttttcc |
| | | gtacacttttggccaggggaccaagttggagatcaga |

MGC26 ANTIBODY

| | | |
|---|---|---|
| 228 | CDRH1 aa | GFTFSTYW |
| 229 | CDRH2 aa | IKQDGTER |
| 230 | CDRH3 aa | VRDRFCRDHMHIEEDLPRPSISPEPATVIPLGSHVTIVCRGPVG |
| | | VETFRLQKESRSLYNDTEDVSQPSPSESEARFRIDSVSEGHGGL |
| | | YRCLYYKSSKWSEQSDYLEMLVKGEDVTWALFPYCGGDGEES |
| | | DYYMDV |
| 231 | CDRL1 aa | QRLSRS |
| 232 | CDRL2 aa | KAS |
| 233 | CDRL2 long aa | LIYKASPLE |
| 234 | CDRL3 aa | QQYSNYSYS |
| 235 | CDRH1 nuc | ggattcacctttagtacttattgg |
| 236 | CDRH2 nuc | ataaagcaagatggaactgagaga |
| 237 | CDRH3 nuc | gtgagagacagattctgcagagatcacatgcacatagaagaagatctgcccagaccc |
| | | tccatctcgccggagccagccaccgtgatccccctggggagccatgtgactatcgtgt |
| | | gccggggcccggttggggttgaaacattccgcctgcagaaggagagtagatccctgta |
| | | caatgacactgaagatgtgtctcaacctagtccatctgagtcagaggccagattccgca |
| | | ttgactcagtaagtgaagggcatggcgggctttatcgctgcctctattataagtcttctaaa |
| | | tggtctgagcagagtgactacctggagatgctggtgaaaggtgaggacgtcacctggg |
| | | ccctgttcccctattgtggtggtgatggagaggaatccgactactacatggacgtc |
| 238 | CDRL1 nuc | cagcgtcttagtcgctcg |
| 239 | CDRL2 nuc | aaggcgtct |
| 240 | CDRL2 long nuc | ctgatctataaggcgtctcctttagaa |
| 241 | CDRL3 nuc | caacaatacagtaattattcatatagt |
| 242 | heavy chain aa | EVQLVDSGGGLVQPGGSLRLSCAASGFTFSTYWMTWVRQTP |
| | | GKGLEWVASIKQDGTERYYVDSVKGRFIISRDNAKNSLYLQM |
| | | HSLRAEDTAVYYCVRDRFCRDHMHIEEDLPRPSISPEPATVIPL |
| | | GSHVTIVCRGPVGVETFRLQKESRSLYNDTEDVSQPSPSESEAR |
| | | FRIDSVSEGHGGLYRCLYYKSSKWSEQSDYLEMLVKGEDVTW |
| | | ALFPYCGGDGEESDYYMDVWGKGTTVTVSS |
| 243 | light chain aa | DIQLTQSPSTLSASVGDRVTISCRASQRLSRSLAWYQQRPRKA |
| | | PNLLIYKASPLEIGGPSRFTGSGSGTEFTLTISSLQPDDSATYYC |
| | | QQYSNYSYSFGQGTKLEIR |
| 244 | heavy chain nuc | gaggtgcagctggtggattctgggggaggcttggtccagcctggggggtccctgagact |
| | | ctcctgtgcagcctctggattcacctttagtacttattggatgacctgggtccgccagact |
| | | ccagggaaggggctggagtgggtggccagcataaagcaagatggaactgagagata |
| | | ctatgtggactctgtgaagggccgattcattatctccagagacaacgccaagaactcac |
| | | tatatttgcaaatgcacagcctgagagccgaggacacggctgtgtattattgtgtgagag |
| | | acagattctgcagagatcacatgcacatagaagaagatctgcccagaccctccatctc |
| | | gccggagccagccaccgtgatccccctggggagccatgtgactatcgtgtgccgggg |
| | | cccggttggggttgaaacattccgcctgcagaaggagagtagatccctgtacaatgac |
| | | actgaagatgtgtctcaacctagtccatctgagtcagaggccagattccgcattgactca |
| | | gtaagtgaagggcatggcgggctttatcgctgcctctattataagtcttctaaatggtctga |
| | | gcagagtgactacctggagatgctggtgaaaggtgaggacgtcacctgggccctgttc |
| | | ccctattgtggtggtgatggagaggaatccgactactacatggacgtctggggcaaagg |
| | | gaccacggtcaccgtctcctca |
| 245 | light chain nuc | gacatccagctgacccagtctccttccaccctgtctgcatctgtaggagacagagtcac |
| | | catctcttgccgggccagtcagcgtcttagtcgctcgaggcctggtatcagcagagacc |
| | | acggaaagcccctaacctcctgatctataaggcgtctcctttagaaattgggggcccat |
| | | caaggttcaccggcagtggatctgggacagaattcactctcaccatcagcagcctgca |
| | | gcctgatgattctgcaacttattactgccaacaatacagtaattattcatatagttttggcca |
| | | ggggaccaagctggagatcaga |

MGC28 ANTIBODY

| | | |
|---|---|---|
| 246 | CDRH1 aa | GFTFSSYW |
| 247 | CDRH2 aa | INQDGSER |
| 248 | CDRH3 aa | ARQRFCSDGSLFHGEDLPRPTISAETGTVISLGSHVTFVCRGPL |
| | | GVQTFRLERESRSRYSETEDVSQVGPSESEARFRIDSVSEGNAG |
| | | LYRCIYYKPPKWSEQSDYLELRVKGEDVTWALLTYCGGDRDE |
| | | SDYYMDV |
| 249 | CDRL1 aa | TGSVTSGSF |
| 250 | CDRL2 aa | STT |
| 251 | CDRL2 long aa | LIYSTTKKH |
| 252 | CDRL3 aa | LLYCGGGQPWV |
| 253 | CDRH1 nuc | ggattcacgtttagttcttattgg |
| 254 | CDRH2 nuc | ataaaccaagatggaagtgagaga |
| 255 | CDRH3 nuc | gcgagacaaagattctgcagtgatgggagtctctttcacggagaagatctgcccagac |
| | | ccaccatctcggctgagacaggcaccgtgatctccctggggagccatgtgactttcgtg |
| | | tgccggggcccacttggggtgcaaacattccgcctggagagggagagtaggtccaga |
| | | tacagtgaaactgaagatgtgtctcaagttggtccatctgagtcagaggccagattccgc |
| | | attgactcagtgagtgaaggaaatgccgggctttatcgatgcatctattacaaaccccct |
| | | aaatggtctgagcagagtgactacctggagctgcgggtgaaaggtgaggacgtcacct |
| | | gggccctgttaacctattgtggtggtgatagagacgaatccgactactacatggacgtc |
| 256 | CDRL1 nuc | actggatcagtcaccagtggttccttt |
| 257 | CDRL2 nuc | agtacaacc |
| 258 | CDRL2 long nuc | ctgatttatagtacaaccaaaaaacac |
| 259 | CDRL3 nuc | ctactctactgtggtggtggtcaaccttgggtg |
| 260 | heavy chain aa | EVQLVESGGGLVQPGGSLRLSCEASGFTESSYWMDWVRQAP |
| | | GKGLEWVANINQDGSERYYVDSVKGRFTISRDTVKNSLYLQ |
| | | MNNLRAEDTAVYYCARQRFCSDGSLFHGEDLPRPTISAETGT |
| | | VISLGSHVTFVCRGPLGVQTERLERESRSRYSETEDVSQVGPSE |
| | | SEARFRIDSVSEGNAGLYRCIYYKPPKWSEQSDYLELRVKGEDV |
| | | TWALLTYCGGDRDESDYYMDVWGKGTTVTVSS |
| 261 | light chain aa | QTVVTQEPSLTVSPGGTVTLTCASSTGSVTSGSFPNWFQQTP |
| | | GQAPRSLIYSTTKKHSWTPARFSGSLLGGKAALTVSDTQPEDE |
| | | AEYYCLLYCGGGQPWVEGGGTKLTVL |
| 262 | heavy chain nuc | gaggtgcagctggtggagtctgggggaggcttggtccagccgggggggtccctgaga |
| | | ctctcctgtgaagcctctggattcacgtttagttcttattggatgacctgggtccgccaggc |
| | | tccagggaaggggctggagtgggtggccaatataaaccaagatggaagtgagagata |
| | | ttatgtggactctgtgaagggccggttcaccatctccagagacaccgtcaagaactcac |
| | | tgtatttgcaaatgaacaacctgagagccgaggacacggctgtatattactgcgcgaga |
| | | caaagattctgcagtgatgggagtctctttcacggagaagatctgcccagacccaccat |
| | | ctcggctgagacaggcaccgtgatctccctggggagccatgtgactttcgtgtgccggg |
| | | gcccacttggggtgcaaacattccgcctggagagggagagtaggtccagatacagtg |
| | | aaactgaagatgtgtctcaagttggtccatctgagtcagaggccagattccgcattgact |
| | | cagtgagtgaaggaaatgccgggctttatcgatgcatctattacaaaccccctaaatggt |
| | | ctgagcagagtgactacctggagctgcgggtgaaaggtgaggacgtcacctgggccc |
| | | tgttaacctattgtggtggtgatagagacgaatccgactactacatggacgtctggggca |
| | | aagggaccacggtcaccgtctcctca |
| 263 | light chain nuc | cagactgtggtgactcaggagccctcactgactgtgtccccaggagggacagtcactc |
| | | tcacctgtgcttccagtactggatcagtcaccagtggttcctttccaaactggttccagca |
| | | gacacctggacaagcacccaggtcactgatttatagtacaaccaaaaaacactcttgg |
| | | acccctgcccggttctcaggctctctccttgggggcaaagctgccctgacagtgtcaga |
| | | tacacagccggaggacgaggctgagtattactgcctactctactgtggtggtggtcaac |
| | | cttgggtgttcggcggagggaccaagctgaccgtccta |

MGC29 ANTIBODY

| | | |
|---|---|---|
| 264 | CDRH1 aa | GFNSRSYW |
| 265 | CDRH2 aa | INQDGTEK |
| 266 | CDRH3 aa | ARDRFCGGESHLHGEEDLPRPSISAEPGTVIPLGSHVTFVCRGP |
| | | VGVHTFRLERGWRYNDTEDVSQAGPSQSEARFRIDSVREGN |
| | | AGLYRCLYYIPPKWSEQSDYLELRVKGGDVTWALLTYCGGD |
| | | GEESDYPMDV |
| 267 | CDRL1 aa | TGPVTSAYY |
| 268 | CDRL2 aa | NIN |
| 269 | CDRL2 long aa | LIYNINKKH |
| 270 | CDRL3 aa | LLSCGGAQPWV |
| 271 | CDRH1 nuc | ggattcaactctcgtagttattgg |
| 272 | CDRH2 nuc | ataaatcaagatggaactgagaaa |
| 273 | CDRH3 nuc | gcgagagacagattctgtggtggtgagagtcacttgcacggagaagaagatctgccca |
| | | gaccctccatctcggctgagccaggcaccgtgatccccctggggagccatgtgacttt |
| | | cgtgtgccggggccccgttggggttcacacattccgcctggagagggggtggagatac |
| | | aacgacactgaagatgtgtctcaagctggtccatctcagtcagaggccagattccgcat |
| | | tgactcggtaagggaaggaaatgccgggctttatcgatgcctctattacataccccctaa |
| | | atggtctgagcagagtgactacctggaactgcgggtgaaaggtggggacgtcacctgg |
| | | gccctgttaacgtactgtggtggtgatggagaggaatccgactaccccatggacgtc |
| 274 | CDRL1 nuc | actggacctgtcaccagtgcttactat |
| 275 | CDRL2 nuc | aatataaac |
| 276 | CDRL2 long nuc | cttatttataatataaacaaaaaacac |
| 277 | CDRL3 nuc | ctgctctcctgtggtggtgctcagccttgggtg |
| 278 | heavy chain aa | EVQLVESGGGLVQPGGSLRLSCEASGFNSRSYWMTWVRQAP |
| | | GKGLEWVATINQDGTEKNYVDSVRGRFTISRDTAKNSLFLQ |
| | | MNSLRAEDTAVYYCARDRFCGGESHLHGEEDLPRPSISAEPGT |
| | | VIPLGSHVTFVCRGPVGVHTFRLERGWRYNDTEDVSQAGPS |
| | | QSEARFRIDSVREGNAGLYRCLYYIPPKWSEQSDYLELRVKGG |
| | | DVTWALLTYCGGDGEESDYPMDVWGKGTTVTVSS |
| 279 | light chain aa | QTVVTQEPSLTVSPGGTVTLTCASNTGPVTSAYYPNWFQQKP |
| | | GQAPRSLIYNINKKHSWTPARFSGSLLGGKAALTLSGVQPEDE |
| | | ADYYCLLSCGGAQPWVFGGGTKLTVQ |
| 280 | heavy chain nuc | gaggtgcagctggtggagtctgggggaggcttggtccagcctggggggtccctgagac |
| | | tctcctgtgaagcctctggattcaactctcgtagttattggatgacctgggtccgccaggc |
| | | tccagggaaggggctggagtgggtggccactataaatcaagatggaactgagaaaaa |
| | | ttatgtggactctgtgaggggccggttcaccatctccagagacaccgccaagaactca |
| | | ctgtttctgcaaatgaacagcctgagagccgaggacacggctgtatattactgcgcgag |
| | | agacagattctgtggtggtgagagtcacttgcacggagaagaagatctgcccagaccc |
| | | tccatctcggctgagccaggcaccgtgatccccctggggagccatgtgactttcgtgtg |
| | | ccggggccccgttggggttcacacattccgcctggagagggggtggagatacaacga |
| | | cactgaagatgtgtctcaagctggtccatctcagtcagaggccagattccgcattgactc |
| | | ggtaagggaaggaaatgccgggctttatcgatgcctctattacataccccctaaatggtc |
| | | tgagcagagtgactacctggaactgcgggtgaaaggtggggacgtcacctgggccct |
| | | gttaacgtactgtggtggtgatggagaggaatccgactaccccatggacgtctggggca |
| | | aagggaccacggtcaccgtctcctca |
| 281 | light chain nuc | cagactgtggtgactcaggagccctcactgactgtgtccccaggagggacagtcactc |
| | | tcacctgtgcttccaacactggacctgtcaccagtgcttactatccaaactggttccagc |
| | | agaagcctggacaagcacccaggtctcttatttataatataaacaaaaaacactcctgg |
| | | acccctgcccggttctcaggctccctccttgggggcaaagctgccctgacactgtcag |
| | | gtgtacagcctgaggacgaggctgactattactgcctgctctcctgtggtggtgctcagc |
| | | cttgggtgttcggcggagggaccaagctgaccgtccaa |

MGC32 ANTIBODY

| | | |
|---|---|---|
| 282 | CDRH1 aa | GFNFRKSW |
| 283 | CDRH2 aa | IREDGSES |
| 284 | CDRH3 aa | ARDRFCNDDEIHRHGQEDLPRPSISAAEGTVIPLGSHVTFVCR |
| | | GPVGVQTFRLEKDSRSIYNDTENVSQPSPSESEARFRIDSVSEG |
| | | NAGLYRCVYYKAPKWSAQSDYLELLVKGQEVTWALFTSCGG |
| | | DGEEPDYDMDV |
| 285 | CDRL1 aa | QSVLYRSKNKNY |
| 286 | CDRL2 aa | STS |
| 287 | CDRL2 long aa | LIYSTSTRA |
| 288 | CDRL3 aa | LQYYITPYT |
| 289 | CDRH1 nuc | gggttcaactttagaaagtcttgg |
| 290 | CDRH2 nuc | ataagagaagatggaagtgagagt |
| 291 | CDRH3 nuc | gcgagagatagattctgcaatgatgatgagattcacagacacggacaagaagatctgc |
| | | ccagaccctccatctcggctgccgaaggcaccgtgatccccctggggagccatgtga |
| | | ctttcgtgtgccggggcccggttggggttcaaacattccgcctggagaaggacagtaga |
| | | tccatatacaatgatactgaaaatgtgtctcaacctagtccatctgagtcagaggccaga |
| | | tttcgcattgactcagtgagtgaaggaaatgccggactttatcggtgcgtctattataagg |
| | | cccctaaatggtctgcgcagagtgattacctggagctgctggtgaaaggtcaggaagtc |
| | | acctgggccctgtttacctcctgtggtggtgatggagaggaacccgactacgacatgga |
| | | cgtc |
| 292 | CDRL1 nuc | cagagtgttttatacaggtccaagaataagaactac |
| 293 | CDRL2 nuc | tcgacatct |
| 294 | CDRL2 long nuc | ctcatttactcgacatctactcgggcg |
| 295 | CDRL3 nuc | ctgcaatattatattactccctacact |
| 296 | heavy chain aa | EVQLVESGGGLVQPGGSLRLSCVASGENFRKSWMGWVRQA |
| | | PGKGLEWVANIREDGSESFYADSVKGRFTVSRDNAKKSLYLHI |
| | | NSLRAEDTAVYYCARDRFCNDDEIHRHGQEDLPRPSISAAEG |
| | | TVIPLGSHVTFVCRGPVGVQTFRLEKDSRSIYNDTENVSQPSPS |
| | | ESEARFRIDSVSEGNAGLYRCVYYKAPKWSAQSDYLELLVKGQ |
| | | EVTWALFTSCGGDGEEPDYDMDVRGKGTTVTVSS |
| 297 | light chain aa | DILMTQSPDSLAVSLGERATINCKSSQSVLYRSKNKNYLAWFQ |
| | | QKPGQPPKVLIYSTSTRASGVPDRFTGSGSGTDFTLTISSLQAE |
| | | DVAVYYCLQYYITPYTFGQGTKLEIK |
| 298 | heavy chain nuc | gaggtgcagctggtggagtctgggggaggcttggtccagccgggggggtccctgaga |
| | | ctctcctgtgtagcctctgggttcaactttagaaagtatggatgggttgggtccgccagg |
| | | ctccagggaaggggctggagtgggtggcaaacataagagaagatggaagtgagagtt |
| | | tctatgcggactctgtgaagggccgcttcaccgtctccagagacaacgccaagaaatc |
| | | actgtatctccatatcaacagcctgagagccgaggacacggctgtctattactgtgcga |
| | | gagatagattctgcaatgatgatgagattcacagacacggacaagaagatctgcccag |
| | | accctccatctcggctgccgaaggcaccgtgatccccctggggagccatgtgactttc |
| | | gtgtgccggggcccggttggggttcaaacattccgcctggagaaggacagtagatcca |
| | | tatacaatgatactgaaaatgtgtctcaacctagtccatctgagtcagaggccagatttc |
| | | gcattgactcagtgagtgaaggaaatgccggactttatcggtgcgtctattataaggccc |
| | | ctaaatggtctgcgcagagtgattacctggagctgctggtgaaaggtcaggaagtcacc |
| | | tgggccctgtttacctcctgtggtggtgatggagaggaacccgactacgacatggacgt |
| | | ccggggcaaagggaccacggtcaccgtctcctca |
| 299 | light chain nuc | gacatcctcatgacccagtctccagactccctggctgtgtctctgggcgagagggcca |
| | | ccatcaactgcaagtccagtcagagtgttttatacaggtccaagaataagaactacttag |
| | | cttggttccagcagaaaccaggacagcctcctaaggtgctcatttactcgacatctactc |
| | | gggcgtccggggtccctgaccgattcactggcagcgggtctgggacagatttcactctc |
| | | accatcagcagcctgcaggctgaagatgtggcagtttattactgtctgcaatattatatta |
| | | ctccctacacttttggccaggggaccaagttggagatcaaa |

MGC33 ANTIBODY

| | | |
|---|---|---|
| 300 | CDRH1 aa | GFTFSSYW |
| 301 | CDRH2 aa | INQDGSER |
| 302 | CDRH3 aa | ARQRFCSDGSLFHGEDLPRPTISAETGTVISLGSHVTFVCRGPL |
| | | GVQTFRLERESRSRYSETEDVSQVGPSESEARFRIDSVSEGNAG |
| | | LYRCIYYKPPKWSEQSDYLELRVKGEDVTWALLTYCGGDRDE |
| | | SDYYMDV |
| 303 | CDRL1 aa | TGSVTSGSF |
| 304 | CDRL2 aa | STT |
| 305 | CDRL2 long aa | LIYSTTKKH |
| 306 | CDRL3 aa | LLYCGGGQPWV |
| 307 | CDRH1 nuc | ggattcacgtttagttcttattgg |
| 308 | CDRH2 nuc | ataaaccaagatggaagtgagaga |
| 309 | CDRH3 nuc | gcgagacaaagattctgcagtgatgggagtctctttcacggagaagatctgcccagac |
| | | ccaccatctcggctgagacaggcaccgtgatctccctggggagccatgtgactttcgtg |
| | | tgccggggcccacttggggtgcaaacattccgcctggagagggagagtaggtccaga |
| | | tacagtgaaactgaagatgtgtctcaagttggtccatctgagtcagaggccagattccgc |
| | | attgactcagtgagtgaaggaaatgccgggctttatcgatgcatctattacaaaccccct |
| | | aaatggtctgagcagagtgactacctggagctgcgggtgaaaggtgaggacgtcacct |
| | | gggccctgttaacctattgtggtggtgatagagacgaatccgactactacatggacgtc |
| 310 | CDRL1 nuc | actggatcagtcaccagtggttccttt |
| 311 | CDRL2 nuc | agtacaacc |
| 312 | CDRL2 long nuc | ctgatttatagtacaaccaaaaaacac |
| 313 | CDRL3 nuc | ctactctactgtggtggtggtcaaccttgggtg |
| 314 | heavy chain aa | EVHLVESGGGLVQPGGSLRLSCEASGFTFSSYWMTWVRQAP |
| | | GKGLEWVANINQDGSERYYVDSVKGRFTISRDTVKNSLYLQ |
| | | MNNLRAEDTAVYYCARQRFCSDGSLFHGEDLPRPTISAETGT |
| | | VISLGSHVTFVCRGPLGVQTFRLERESRSRYSETEDVSQVGPSE |
| | | SEARFRIDSVSEGNAGLYRCIYYKPPKWSEQSDYLELRVKGEDV |
| | | TWALLTYCGGDRDESDYYMDVWGKGTTVTVSS |
| 315 | light chain aa | QTVVTQEPSLTVSPGGTVTLTCASSTGSVTSGSFPNWFQQTP |
| | | GQAPRSLIYSTTKKHSWTPARFSGSLLGGKAALTVSDTQPEDE |
| | | AEYYCLLYCGGGQPWVFGGGTKLTVQ |
| 316 | heavy chain nuc | gaggtgcacctggtggagtctgggggaggcttggtccagccgggggggtccctgaga |
| | | ctctcctgtgaagcctctggattcacgtttagttatattggatgacctgggtccgccaggc |
| | | tccagggaaggggctggagtgggtggccaatataaaccaagatggaagtgagagata |
| | | ttatgtggactctgtgaagggccggttcaccatctccagagacaccgtcaagaactcac |
| | | tgtatttgcaaatgaacaacctgagagccgaggacacggctgtatattactgcgcgaga |
| | | caaagattctgcagtgatgggagtctctttcacggagaagatctgcccagacccaccat |
| | | ctcggctgagacaggcaccgtgatctccaggggagccatgtgactttcgtgtgccggg |
| | | gcccacttggggtgcaaacattccgcctggagagggagagtaggtccagatacagtg |
| | | aaactgaagatgtgtctcaagttggtccatctgagtcagaggccagattccgcattgact |
| | | cagtgagtgaaggaaatgccgggctttatcgatgcatctattacaaaccccctaaatggt |
| | | ctgagcagagtgactacctggagctgcgggtgaaaggtgaggacgtcacctgggccc |
| | | tgttaacctattgtggtggtgatagagacgaatccgactactacatggacgtctggggca |
| | | aagggaccacggtcaccgtctcctca |
| 317 | light chain nuc | cagactgtggtgactcaggagccctcactgactgtgtccccaggagggacagtcactc |
| | | tcacctgtgcttccagtactggatcagtcaccagtggttcctttccaaactggttccagca |
| | | gacacctggacaagcacccaggtcactgatttatagtacaaccaaaaaacactcttgg |
| | | acccctgcccggttctcaggctctctccttgggggcaaagctgccctgacagtgtcaga |
| | | tacacagccggaggacgaggctgagtattactgcctactctactgtggtggtggtcaac |
| | | cttgggtgttcggcggagggaccaagctgaccgtccaa |

MGC34 ANTIBODY

| | | |
|---|---|---|
| 318 | CDRH1 aa | GFTFSSYW |
| 319 | CDRH2 aa | INQDGSQK |
| 320 | CDRH 3 aa | ARERLCTDDSHMHGEEDLPRPSISAEPGTVIPLGSHVTFVCRG |
| | | PVGIHTFRLERESRSLYTETEDVTQVSPSESEARFRIESVTEGNAG |
| | | LYRCVYYKPPKWSEQSDYLELLVKGEDVTRALFTHCGGDGKE |
| | | SDYHMDV |
| 321 | CDRL1 aa | TGAVTSGYY |
| 322 | CDRL2 aa | STS |
| 323 | CDRL2 long aa | LIYSTSKTH |
| 324 | CDRL3 aa | LLYYGGPQPWV |
| 325 | CDRH1 nuc | ggattcacctttagtagttattgg |
| 326 | CDRH2 nuc | ataaaccaagatggaagtcagaaa |
| 327 | CDRH3 nuc | gcgagagaaagattgtgcactgatgatagtcacatgcacggagaagaagatctgccc |
| | | agaccctccatctcggctgagccaggcaccgtgatccccctggggagtcatgtgacct |
| | | tcgtgtgccggggcccggttgggattcacacattccgcctggagagggagagtagatc |
| | | cctatacactgaaactgaagatgtgactcaagtaagtccttctgagtcagaggccagatt |
| | | ccgcattgagtcagtaactgaaggaaatgccgggctttatcgctgcgtctattataagcc |
| | | ccctaaatggtctgagcagagtgactacctggagctgctggtgaaaggtgaggacgtc |
| | | acccgggccctgttcacccactgtggtggtgatggaaaggagtccgactaccacatgg |
| | | acgtc |
| 328 | CDRL1 nuc | actggagcagtcaccagtggttactat |
| 329 | CDRL2 nuc | agtacaagc |
| 330 | CDRL2 long nuc | ctgatttatagtacaagcaaaacacac |
| 331 | CDRL3 nuc | ctgctctattatggtggtcctcagccttgggtg |
| 332 | heavy chain aa | EVQLVESGGGLVQPGGSLRLSCEASGFTFSSYWMSWVRQAP |
| | | GKGLEWVANINQDGSQKDYVDSVKGRFTISRDTAKNSLYLQ |
| | | MNSLRAEDTAVYYCARERLCTDDSHMHGEEDLPRPSISAEPG |
| | | TVIPLGSHVTFVCRGPVGIHTFRLERESRSLYTETEDVTQVSPSE |
| | | SEARFRIESVTEGNAGLYRCVYYKPPKWSEQSDYLELLVKGED |
| | | VTRALFTHCGGDGKESDYHMDVWGKGTTVTVSS |
| 333 | light chain aa | QTVVTQEPSLTVSPGGTVTLTCASSTGAVTSGYYPSWFHQKP |
| | | GQPVRALIYSTSKTHSWTPARFSGSLLGGKAALTLSNVQPEDE |
| | | ADYYCLLYYGGPQPWVFGGGTKLTVQ |
| 334 | heavy chain nuc | gaggtgcagctggtggagtctgggggaggcttggtccagcctggggggtcactgagac |
| | | tctcctgtgaagcctccggattcacctttagtagttattggatgagctgggtccgccaggc |
| | | tccagggaaggggctggagtgggtggccaatataaaccaagatggaagtcagaaag |
| | | attatgtggattctgtgaagggccgattcaccatctccagagacaccgccaagaattcat |
| | | tatatctccaaatgaacagcctgagagccgaggacacggctgtttactactgtgcgaga |
| | | gaaagattgtgcactgatgatagtcacatgcacggagaagaagatctgcccagaccct |
| | | ccatctcggctgagccaggcaccgtgatccccctggggagtcatgtgaccttcgtgtgc |
| | | cggggcccggttgggattcacacattccgcctggagagggagagtagatccctataca |
| | | ctgaaactgaagatgtgactcaagtaagtccttctgagtcagaggccagattccgcattg |
| | | agtcagtaactgaaggaaatgccgggctttatcgctgcgtctattataagccccctaaat |
| | | ggtctgagcagagtgactacctggagctgctggtgaaaggtgaggacgtcacccggg |
| | | ccctgttcacccactgtggtggtgatggaaaggagtccgactaccacatggacgtctgg |
| | | ggcaaagggaccacggtcaccgtctcctca |
| 335 | light chain nuc | cagactgtggtgactcaggagccctcactgactgtgtccccaggagggacagtcactc |
| | | tcacctgtgcttctagcactggagcagtcaccagtggttactatccaagctggttccacc |
| | | agaaacctggacaaccagtcagggcactgatttatagtacaagcaaaacacactcct |
| | | ggacccctgcccgcttctcaggctccctccttgggggcaaagctgccctgacactgtc |
| | | aaatgtccagcctgaggacgaggctgactattactgcctgctctattatggtggtcctca |
| | | gccttgggtgttcggcggagggaccaagctgaccgtccaa |

MGC35 ANTIBODY

| | | |
|---|---|---|
| 336 | CDRH1 aa | GFNSRSYW |
| 337 | CDRH2 aa | INQDATEK |
| 338 | CDRH3 aa | ARDRFCGGESHLHGQEDLPRPSISAEPGSVIPLGSLVTFVCRGP |
| | | VGVHTFRLERGWTYNDTEDVSQAGPSESEARFRMDSVREGN |
| | | AGLYRCIYYKPPKWSEQSAYLELRVKGGDVTWALLTYCGGD |
| | | GEESDYPMDV |
| 339 | CDRL1 aa | TGPVTSAYY |
| 340 | CDRL2 aa | NIN |
| 341 | CDRL2 long aa | LIYNINKKH |
| 342 | CDRL3 aa | LLSCGGAQPWV |
| 343 | CDRH1 nuc | ggattcaactctcgtagttattgg |
| 344 | CDRH2 nuc | ataaatcaagatgcaactgagaaa |
| 345 | CDRH3 nuc | gcgagagacagattctgtggtggtgagagtcacttgcacggacaagaagatctgccca |
| | | gaccctccatctcggctgagccaggctccgtgatccccaggggagccttgtgactttc |
| | | gtgtgccggggcccggttggggttcacacattccgcctcgagagggggtggacataca |
| | | acgacactgaagatgtgtctcaagctggtccatctgagtcagaggccagattccgcatg |
| | | gactcggtaagggaaggaaatgccgggctttatcgatgcatctattacaaaccccctaa |
| | | atggtctgagcagagtgcctacctggaactgcgggtgaaaggtggggacgtcacctgg |
| | | gccctgttaacgtactgtggtggtgatggagaggaatccgactaccccatggacgtc |
| 346 | CDRL1 nuc | actggacctgtcaccagtgcttactat |
| 347 | CDRL2 nuc | aatataaac |
| 348 | CDRL2 long nuc | cttatttataatataaacaaaaaacac |
| 349 | CDRL3 nuc | ctgctctcctgtggtggtgctcagccttgggtg |
| 350 | heavy chain aa | EVQLVESGGGLVQPGGSLRLSCEASGFNSRSYWMTWVRQAP |
| | | GKGLEWVASINQDATEKNYVDSVKGRFTISRDTAKNSLYLQM |
| | | NSLRAEDTAVYYCARDRFCGGESHLHGQEDLPRPSISAEPGSV |
| | | IPLGSLVTFVCRGPVGVHTFRLERGWTYNDTEDVSQAGPSESE |
| | | ARFRMDSVREGNAGLYRCIYYKPPKWSEQSAYLELRVKGGDV |
| | | TWALLTYCGGDGEESDYPMDVWGKGTTVTVSS |
| 351 | light chain aa | QTVVTQEPSLTVSPGGTVTLTCASSTGPVTSAYYPNWFQQKP |
| | | GQAPRSLIYNINKKHSWTPDRFSGSLLGGKAALTLSGVQPEDE |
| | | ADYYCLLSCGGAQPWVFGGGTKLTVQ |
| 352 | heavy chain nuc | gaggtgcaactggtggagtctgggggaggcttggtccagcctggggggtccctgagac |
| | | tctcctgtgaagcctctggattcaactctcgtagttattggatgacctgggtccgccaggc |
| | | tccagggaaggggctggagtgggtggccagtataaatcaagatgcaactgagaaaaa |
| | | ttatgtggactctgtgaagggccggttcaccatctccagagacaccgccaagaactca |
| | | ctgtatctgcaaatgaacagcctgagagccgaggacacggctgtatattactgcgcga |
| | | gagacagattctgtggtggtgagagtcacttgcacggacaagaagatctgcccagacc |
| | | ctccatctcggctgagccaggctccgtgatccccctggggagccagtgactttcgtgtg |
| | | ccggggcccggttggggttcacacattccgcctcgagagggggtggacatacaacga |
| | | cactgaagatgtgtctcaagctggtccatctgagtcagaggccagattccgcatggact |
| | | cggtaagggaaggaaatgccgggctttatcgatgcatctattacaaaccccctaaatgg |
| | | tctgagcagagtgcctacctggaactgcgggtgaaaggtggggacgtcacctgggcc |
| | | ctgttaacgtactgtggtggtgatggagaggaatccgactaccccatggacgtctgggg |
| | | caaagggaccacggtcaccgtctcctca |
| 353 | light chain nuc | cagactgtggtgactcaggagccctcactgactgtgtccccaggagggacagtcactc |
| | | tcacctgtgcttccagcactggacctgtcaccagtgcttactatccaaactggttccagc |
| | | agaagcctggacaagcacccaggtctcttatttataatataaacaaaaaacactcctgg |
| | | acccctgaccggttctcaggctccctccttgggggcaaagctgccctgacactgtcag |
| | | gtgtacagcctgaggacgaggctgactattactgcctgctctcctgtggtggtgctcagc |
| | | cttgggtgttcggcggagggaccaagctgaccgtccaa |

MGC36 ANTIBODY

| | | |
|---|---|---|
| 354 | CDRH1 aa | GFNSRSYW |
| 355 | CDRH2 aa | INQDGTEK |
| 356 | CDRH3 aa | ARDRFCGGESHLHGEEDLPRPSISAEPDTVIPLGSHVTFVCRGP |
| | | VGVHTFRLERGWRYNDTEDVSQAGPSESEARFRIDSVREGNA |
| | | GLYRCIYYIAPKWSEQSDYLELRVKGGDVTWALLTYCGGDGE |
| | | ESDYPMDV |
| 357 | CDRL1 aa | TGPVTSAYY |
| 358 | CDRL2 aa | SIN |
| 359 | CDRL2 long aa | LIYSINKKH |
| 360 | CDRL3 aa | LLSCGGAQPWV |
| 361 | CDRH1 nuc | ggattcaactctcgtagttattgg |
| 362 | CDRH2 nuc | ataaatcaagatgggactgagaaa |
| 363 | CDRH3 nuc | gcgagagacagattctgtggtggtgagagtcacttgcacggagaagaagatctgccca |
| | | gaccctccatctcggctgagccagacaccgtaatccccctggggagccatgtgacttt |
| | | cgtgtgccggggcccggttggggttcacacattccgcctggagagggggtggaggtac |
| | | aacgacactgaagatgtgtctcaagctggtccatctgagtcagaggccagattccgcat |
| | | tgactcggtaagggaaggaaatgccgggctttatcgatgcatctattacatagcccctaa |
| | | atggtctgagcagagtgactacctggagctgcgggtgaaaggtggggacgtcacctgg |
| | | gccctgttaacgtactgtggcggtgatggagaggaatccgactaccccatggacgtc |
| 364 | CDRL1 nuc | actggacctgtcaccagtgcttactat |
| 365 | CDRL2 nuc | agtataaac |
| 366 | CDRL2 long nuc | cttatttatagtataaacaaaaaacac |
| 367 | CDRL3 nuc | ctgctctcctgtggtggtgctcagccttgggtg |
| 368 | heavy chain aa | EVVLVESGGGLVQPGGSLRLSCEASGFNSRSYWMTWVRQAP |
| | | GKGLEWVASINQDGTEKNYVDSVKGRFTISRDSAKNSLYLQ |
| | | MSSLRADDTAVYYCARDRFCGGESHLHGEEDLPRPSISAEPDT |
| | | VIPLGSHVTFVCRGPVGVHTFRLERGWRYNDTEDVSQAGPSE |
| | | SEARFRIDSVREGNAGLYRCIYYIAPKWSEQSDYLELRVKGGD |
| | | VTWALLTYCGGDGEESDYPMDVWGKGTTVTVSS |
| 369 | light chain aa | QTVVTQEPSLTVSPGGTVTLTCASSTGPVTSAYYPNWFQQKP |
| | | GQAPRSLIYSINKKHSWTPARFSGSLLGGKAALTLSGVQPEDE |
| | | ADYYCLLSCGGAQPWVFGGGTKLTVQ |
| 370 | heavy chain nuc | gaggtggtactggtggagtctgggggaggcttggtccagcctggggggtccctgagact |
| | | ctcctgtgaagcctctggattcaactctcgtagttattggatgacctgggtccgccaggct |
| | | ccagggaaggggctggagtgggtggccagtataaatcaagatgggactgagaaaaat |
| | | tatgtggactctgtgaagggccggttcaccatctccagagactccgccaagaactcact |
| | | gtatctgcaaatgagcagcctgagagccgacgacacggctgtatattactgtgcgaga |
| | | gacagattctgtggtggtgagagtcacttgcacggagaagaagatctgcccagaccct |
| | | ccatctcggctgagccagacaccgtaatccccctggggagccatgtgactttcgtgtgc |
| | | cggggcccggttggggttcacacattccgcctggagagggggtggaggtacaacgac |
| | | actgaagatgtgtctcaagctggtccatctgagtcagaggccagattccgcattgactcg |
| | | gtaagggaaggaaatgccgggctttatcgatgcatctattacatagcccctaaatggtct |
| | | gagcagagtgactacctggagctgcgggtgaaaggtggggacgtcacctgggccctg |
| | | ttaacgtactgtggcggtgatggagaggaatccgactaccccatggacgtctggggca |
| | | aagggaccacggtcaccgtctcctca |
| 371 | light chain nuc | cagactgtggttactcaggagccctcactgactgtgtccccaggagggacagtcactct |
| | | cacctgtgcttccagcactggacctgtcaccagtgcttactatccaaactggttccagca |
| | | gaagcctggacaagcacccaggtctcttatttatagtataaacaaaaaacactcctgga |
| | | cccctgcccggactcaggctccctccttgggggcaaagctgccctgacactgtcaggt |
| | | gtacagcctgaggacgaggctgactattactgcctgctctcctgtggtggtgctcagcct |
| | | tgggtgttcggcggagggaccaagctgaccgtccaa |

MGC37 ANTIBODY

| | | |
|---|---|---|
| 372 | CDRH1 aa | GFTFRNYW |
| 373 | CDRH2 aa | IRQDGSEK |
| 374 | CDRH3 aa | VRDKFCSDENHMHVADDLPRPSISPEPGTVIPLGSHVTFVCRG |
| | | PVGVQTFRLEKDRRSTYNDTEDVSQPSPSESEARFRIDSVTEGN |
| | | AGLYRCVYYKPPKWSDQSDFLELLVKGEDVTWALFPHCGAD |
| | | GEDSDYYMDV |
| 375 | CDRL1 aa | QRLSRS |
| 376 | CDRL2 aa | KAS |
| 377 | CDRL2 long aa | LIYKASPLE |
| 378 | CDRL3 aa | QQYSNYSYS |
| 379 | CDRH1 nuc | ggattcaccttcagaaattattgg |
| 380 | CDRH2 nuc | ataaggcaagatggaagtgagaag |
| 381 | CDRH3 nuc | gtgagagataaattctgcagtgatgagaatcacatgcacgtagcagatgatctgcccag |
| | | accctctatctcgcctgagccaggcaccgtgatccccctggggagccatgtgactttcg |
| | | tgtgtcggggcccggttggggttcaaacattccgcctggagaaggacagaagatccac |
| | | atacaatgatactgaagatgtgtctcaacctagtccatctgagtcagaggccagattccg |
| | | cattgactcagtaactgaaggaaatgccgggctttatcgctgcgtctattataagccccc |
| | | taaatggtctgaccagagtgacttcctggagttgctggtgaagggtgaggacgtcacctg |
| | | ggccctgttcccccattgtggtgctgatggagaggactccgactactacatggacgtc |
| 382 | CDRL1 nuc | cagcgtcttagtcgctcg |
| 383 | CDRL2 nuc | aaggcgtct |
| 384 | CDRL2 long nuc | ctgatctataaggcgtctcctttagaa |
| 385 | CDRL3 nuc | caacaatacagtaattattcatatagt |
| 386 | heavy chain aa | EVQLVESGGDLVQPGGSLRLSCAASGFTERNYWMSWVRQTP |
| | | GKGLEWVANIRQDGSEKYYVDSVKGRFTISRDNAKNLLYLQ |
| | | MNSLRAEDTAVYYCVRDKFCSDENHMHVADDLPRPSISPEPG |
| | | TVIPLGSHVTFVCRGPVGVQTFRLEKDRRSTYNDTEDVSQPSP |
| | | SESEARFRIDSVTEGNAGLYRCVYYKPPKWSDQSDFLELLVKG |
| | | EDVTWALFPHCGADGEDSDYYMDVWGKGTTVTVSS |
| 387 | light chain aa | DIQLTQSPSTLSASVGDRVTISCRASQRLSRSLAWYQQRPRKA |
| | | PNLLIYKASPLEIGGPSRFTGSGSGTEFTLTISSLQPDDSATYYC |
| | | QQYSNYSYSFGQGTKLEIR |
| 388 | heavy chain nuc | gaggtgcagctggtggagtctgggggagacttggtccagcctggggggtccctgagac |
| | | tctcctgtgcagcctctggattcaccttcagaaattattggatgagttgggtccgccagac |
| | | tccagggaagggactggagtgggtggccaacataaggcaagatggaagtgagaagt |
| | | attatgtggactctgtgaagggccgattcaccatctccagagacaacgccaagaactta |
| | | ttatatctacaaatgaacagcctgagagccgaggacacggctgtgtattactgtgtgaga |
| | | gataaattctgcagtgatgagaatcacatgcacgtagcagatgatctgcccagaccctc |
| | | tatctcgcctgagccaggcaccgtgatccccctggggagccatgtgactttcgtgtgtcg |
| | | gggcccggttggggttcaaacattccgcctggagaaggacagaagatccacatacaa |
| | | tgatactgaagatgtgtctcaacctagtccatctgagtcagaggccagattccgcattga |
| | | ctcagtaactgaaggaaatgccgggctttatcgctgcgtctattataagccccctaaatg |
| | | gtctgaccagagtgacttcctggagttgctggtgaagggtgaggacgtcacctgggccc |
| | | tgttcccccattgtggtgctgatggagaggactccgactactacatggacgtctggggca |
| | | aagggaccacggtcaccgtctcctca |
| 389 | light chain nuc | gacatccagctgacccagtctccttccaccctgtctgcatctgtaggagacagagtcac |
| | | catctcttgccgggccagtcagcgtcttagtcgctcgttggcctggtatcagcagagacc |
| | | acggaaagcccctaacctcctgatctataaggcgtctcctttagaaattgggggcccat |
| | | caaggttcaccggcagtggatctgggacagaattcactctcaccatcagcagcctgca |
| | | gcctgatgattctgcaacttattactgccaacaatacagtaattattcatatagttttggcca |
| | | ggggaccaagctggagatcaga |

MGD21 ANTIBODY

| | | |
|---|---|---|
| 390 | CDRH1 aa | GDYVNTNRR |
| 391 | CDRH2 aa | VHQSGRT |
| 392 | CDRH3 aa | ARASPLKSQRDTDLPRPSISAEPGTVIPLGSHVTFVCRGPVGV |
| | | QTFRLERERNYLYSDTEDVSQTSPSESEARFRIDSVNAGNAGLF |
| | | RCIYYKSRKWSEQSDYLELVVKGEDVTWALSQSQDDPRACP |
| | | QGELPISTDIYYVDV |
| 393 | CDRL1 aa | QHINDS |
| 394 | CDRL2 aa | GAS |
| 395 | CDRL2 long aa | LIYGASNLH |
| 396 | CDRL3 aa | QQCNCFPPD |
| 397 | CDRH1 nuc | ggtgactacgtcaatactaataggagg |
| 398 | CDRH2 nuc | gttcatcaaagtgggagaacc |
| 399 | CDRH3 nuc | gcgagagcgtctccactcaaatctcagagggacaccgatctgcccagaccctccatc |
| | | tcggctgagccgggcaccgtgatccccctggggagccatgtgactttcgtgtgccggg |
| | | gcccggttggggttcaaacattccgcctggagagggagaggaattatttatacagtgata |
| | | ctgaagatgtgtctcaaactagtccatctgagtcggaggccagattccgcattgactcag |
| | | taaatgcaggcaatgccgggctattcgctgcatctattacaagtcccgtaaatggtctga |
| | | gcagagtgactacctggagctggtggtgaaaggtgaggacgtcacctgggccctgtcc |
| | | cagtctcaagacgaccctcgagcttgtccccagggggagctccccataagtaccgat |
| | | atttactacgtggacgtc |
| 400 | CDRL1 nuc | caacatattaatgattct |
| 401 | CDRL2 nuc | ggtgcatcc |
| 402 | CDRL2 long nuc | ctgatatatggtgcatccaatttgcac |
| 403 | CDRL3 nuc | caacagtgtaattgtttccctccggac |
| 404 | heavy chain aa | EVQLVETGPGLMKTSGTLSLTCAVSGDYVNTNRRWSWVRQ |
| | | APGKGLEWIGEVHQSGRTNYNPSLKSRVTISVDKSKNQFSLKV |
| | | DSVTAADTAVYYCARASPLKSQRDTDLPRPSISAEPGTVIPLGS |
| | | HVTFVCRGPVGVQTFRLERERNYLYSDTEDVSQTSPSESEARF |
| | | RIDSVNAGNAGLFRCIYYKSRKWSEQSDYLELVVKGEDVTWA |
| | | LSQSQDDPRACPQGELPISTDIYYVDVWGNGTTVTVSS |
| 405 | light chain aa | AIRMTQSPSSLSASPGDKVSITCRASQHINDSLAWFQQRPGK |
| | | APKLLIYGASNLHSGVPSRFSGTGSGTDFTLTITGLQSEDFATY |
| | | FCQQCNCFPPDFGQGTRLEIK |
| 406 | heavy chain nuc | gaggtgcagctggtggagacgggcccaggactgatgaagacttcggggaccctgtcc |
| | | ctcacgtgcgctgtgtctggtgactacgtcaatactaataggaggtggagttgggtccgc |
| | | caggccccagggaagggcctggagtggattggagaggttcatcaaagtgggagaac |
| | | caattacaacccgtccctcaagagccgagtcaccatatcagtagacaagtctaagaat |
| | | cagttctctctgaaggtggactctgtgaccgccgcggacacggccgtgtattactgtgcg |
| | | agagcgtctccactcaaatctcagagggacaccgatctgcccagaccctccatctcg |
| | | gctgagccgggcaccgtgatccccctggggagccatgtgactttcgtgtgccggggcc |
| | | cggaggggttcaaacattccgcctggagagggagaggaattatttatacagtgatactg |
| | | aagatgtgtctcaaactagtccatctgagtcggaggccagattccgcattgactcagtaa |
| | | atgcaggcaatgccgggctttttcgctgcatctattacaagtcccgtaaatggtctgagc |
| | | agagtgactacctggagctggtggtgaaaggtgaggacgtcacctgggccctgtccca |
| | | gtctcaagacgaccctcgagcttgtccccagggggagctccccataagtaccgatattt |
| | | actacgtggacgtctggggcaacgggaccacggtcaccgtctcctca |
| 407 | light chain nuc | gccatccggatgacccagtctccatcctcactctctgcatcaccaggggacaaagtca |
| | | gcatcacttgtcgggcgagtcaacatattaatgattattggcctggtttcaacaaaggcc |
| | | agggaaagccccaaaactcctgatatatggtgcatccaatttgcacagtggggtcccat |
| | | cgaggttcagcggcactgggtcagggacagatttcactctcactatcaccggcctgca |
| | | gtctgaagattttgcaacttatttctgtcaacagtgtaattgtttccctccggacttcggcca |
| | | agggacacgactggagattaaa |

MGD23 ANTIBODY

| | | |
|---|---|---|
| 408 | CDRH1 aa | GGSISSNKW |
| 409 | CDRH2 aa | VYQTGIT |
| 410 | CDRH3 aa | ATISQLRPQGDTEDLPRPSLSAEPGIVIPLGSHVTFVCRGPAGV |
| | | ETFRLERESRFTYNDTEDVSQASPSESEARFRIDSVSEGNAGPYR |
| | | CLYYKARKWSDQSDYLELLVKGADVTWALPQSQLAPRACPQ |
| | | GELRISTDVFSMNV |
| 411 | CDRL1 aa | QYVGNY |
| 412 | CDRL2 aa | GVS |
| 413 | CDRL2 long aa | LIHGVSTLQ |
| 414 | CDRL3 aa | QQYYTSPPD |
| 415 | CDRH1 nuc | ggtggctccattagtagtaataagtgg |
| 416 | CDRH2 nuc | gtgtatcagactggtattacc |
| 417 | CDRH3 nuc | gcgacaatttctcaactgaggccgcagggggacaccgaagatctgcccagaccctc |
| | | cctctcggctgaaccaggcaccgtgatccccctggggagtcacgtgactttcgtgtgcc |
| | | ggggcccggctggggtcgaaacattccgcctggagagggagagtagattcacttaca |
| | | acgatactgaagatgtgtctcaagcgagtccatctgagtcagaggccagattccgcatt |
| | | gactcagtaagtgaaggaaatgccgggccttatcgctgcctctattataaggcccgtaa |
| | | atggtctgaccagagtgactacttggaattgctggtgaagggtgcggacgtcacctggg |
| | | ccctgccccagtctcagctcgcccctcgagcttgtccccagggagaactccgcattag |
| | | taccgatgttttctccatgaacgtc |
| 418 | CDRL1 nuc | caatatgttgggaattat |
| 419 | CDRL2 nuc | ggtgtatcc |
| 420 | CDRL2 long nuc | ctcattcacggtgtatccactttgcaa |
| 421 | CDRL3 nuc | cagcagtattatacttcccctccggac |
| 422 | heavy chain aa | QVQLQESGPGLVKPSGTLSLTCSVSGGSISSNKWWSWVRQS |
| | | PGKGLEWIGEVYQTGITNYNPSLKGRVTMSVDKSKNQFSLRL |
| | | TSVTAADTAVYYCATISQLRPQGDTEDLPRPSLSAEPGTVIPLG |
| | | SHVTFVCRGPAGVETFRLERESRFTYNDTEDVSQASPSESEARF |
| | | RIDSVSEGNAGPYRCLYYKARKWSDQSDYLELLVKGADVTW |
| | | ALPQSQLAPRACPQGELRISTDVFSMNVWGNGTTVTVSS |
| 423 | light chain aa | AVRVTQSPTSLSASTGDRVTITCRTSQYVGNYLDWYQQKPG |
| | | KAPKLLIHGVSTLQNGVPSRFSGSASGTDFTLNITCLQSEDSAT |
| | | YYCQQYYTSPPDFGQGTRLEIK |
| 424 | heavy chain nuc | caggtgcagctgcaggagtcgggcccaggactggtgaagccttcgggaaccctgtcc |
| | | ctcacctgcagtgtctctggtggctccattagtagtaataagtggtggagttgggtccgcc |
| | | agtccccagggaagggcctggagtggattggggaggtgtatcagactggtattaccaa |
| | | ctacaacccgtccctcaagggtcgagtcaccatgtcagtggacaagtccaagaacca |
| | | attctccctgagactgacactgtgaccgccgcggacacggccgtgtattactgtgcgac |
| | | aatttctcaactgaggccgcagggggacaccgaagatctgcccagaccctccctctc |
| | | ggctgaaccaggcaccgtgatccccctggggagtcacgtgactttcgtgtgccggggc |
| | | ccggctggggtcgaaacattccgcctggagagggagagtagattcacttacaacgata |
| | | ctgaagatgtgtctcaagcgagtccatctgagtcagaggccagattccgcattgactca |
| | | gtaagtgaaggaaatgccgggccttatcgctgcctctattataaggcccgtaaatggtct |
| | | gaccagagtgactacttggaattgctggtgaagggtgcggacgtcacctgggccctgc |
| | | cccagtctcagctcgcccctcgagcttgtccccagggagaactccgcattagtaccga |
| | | tgattttctccatgaacgtctggggcaacgggaccacggtcaccgtctcttca |
| 425 | light chain nuc | gccgtccgggtgacccagtctccaacctcactgtctgcatctacaggagacagagtca |
| | | ccatcacttgtcggacgagtcaatatgttgggaattatttagattggtatcagcaaaaacc |
| | | agggaaagcccctaaactcctcattcacggtgtatccactttgcaaaatggggtcccat |
| | | caaggttcagtggcagtgcctccgggacagacttcactctcaacatcacctgcctaca |
| | | gtctgaagattctgcaacttattactgtcagcagtattatacttcccctccggacttcggcc |
| | | aagggacacgcctggaaattaag |

MGD30 ANTIBODY

| | | |
|---|---|---|
| 426 | CDRH1 aa | GGSITSSKW |
| 427 | CDRH2 aa | IYHNGTT |
| 428 | CDRH3 aa | ATASPFKSHHRTFEKLPRPSISAEPGTVIPLGSRVTFVCRGPVGV |
| | | QTERLERETSFTYNDTEDVSQVSPSESEARFRIDSVSEGYAGPYR |
| | | CVYYKAPKWSEQSDYLDLLVKGEDVTWALTQPQLDPRACPQ |
| | | GDLRMSTDIYCMDV |
| 429 | CDRL1 aa | QDISTF |
| 430 | CDRL2 aa | AAS |
| 431 | CDRL2 long aa | LIFAASTLQ |
| 432 | CDRL3 aa | QQYYCFPPD |
| 433 | CDRH1 nuc | ggtggctccatcaccagtagtaagtgg |
| 434 | CDRH2 nuc | atctatcataatgggaccacc |
| 435 | CDRH3 nuc | gcaacggcgtctcccttcaagtctcatcacaggaccaccgaaaaactgcccagacc |
| | | ctccatctcggctgagccgggcaccgtgatccccctggggagccgtgtgactttcgtgt |
| | | gccggggcccggttggggttcaaacattccgcctagagagggagactagctttacatat |
| | | aatgatactgaagatgtgtctcaggttagtccgtctgagtcagaggccagattccgcatt |
| | | gactcagtgagtgagggatatgccgggccttatcgctgcgtctattataaggcccctaag |
| | | tggtccgagcagagtgactacctggacctgctggtgaaaggtgaggacgtcacttggg |
| | | ccctgacccagcctcagctcgaccctcgagcttgtccccagggggacctccgcatga |
| | | gcaccgatatttactgcatggacgtc |
| 436 | CDRL1 nuc | caggatattagcactttt |
| 437 | CDRL2 nuc | gctgcatct |
| 438 | CDRL2 long nuc | ctaatctttgctgcatctactttacaa |
| 439 | CDRL3 nuc | caacagtattattgtttccctccggac |
| 440 | heavy chain aa | QVQLQESGPGLVKPSETLSLSCAVTGGSITSSKWWTWVRQG |
| | | PDKGLEWIGKIYHNGTTNYNPSLKSRVAMSVDKSRNQFSLRL |
| | | TSVTAADTALYYCATASPFKSHHRTTEKLPRPSISAEPGTVIPLG |
| | | SRVTFVCRGPVGVQTFRLERETSFTYNDTEDVSQVSPSESEARF |
| | | RIDSVSEGYAGPYRCVYYKAPKWSEQSDYLDLLVKGEDVTWA |
| | | LTQPQLDPRACPQGDLRMSTDIYCMDVWGKGTTVTVSS |
| 441 | light chain aa | AIRLTQSPSSLSASIGDRVTITCRASQDISTFLAWYQQESGKAP |
| | | RLLIFAASTLQTGVPSRFSGSGSGTDFTLTISGLQSEDFATYYCQ |
| | | QYYCFPPDFGQGTRLDIK |
| 442 | heavy chain nuc | caggtgcagctgcaggagtcgggcccaggactggtgaagccttcagaaaccctgtcc |
| | | ctctcctgcgctgtcactggtggctccatcaccagtagtaagtggtggacttgggtccgc |
| | | cagggcccagataaggggctggagtggattgggaaaatctatcataatgggaccacc |
| | | aactacaatccgtccctcaagagtcgagtcgccatgtcggtggacaagtccaggaac |
| | | cagttctccctgagactgacctccgtgaccgccgcggacacggccttgtattactgtgc |
| | | aacggcgtctcccttcaagtctcatcacaggaccaccgaaaaactgcccagaccctc |
| | | catctcggctgagccgggcaccgtgatccccctggggagccgtgtgactttcgtgtgcc |
| | | ggggcccggttggggttcaaacattccgcctagagagggagactagctttacatataat |
| | | gatactgaagatgtgtctcaggttagtccgtctgagtcagaggccagattccgcattgac |
| | | tcagtgagtgagggatatgccgggccttatcgctgcgtctattataaggcccctaagtgg |
| | | tccgagcagagtgactacctggacctgctggtgaaaggtgaggacgtcacttgggccc |
| | | tgacccagcctcagctcgaccctcgagcttgtccccagggggacctccgcatgagca |
| | | ccgatatttactgcatggacgtctggggcaaagggaccacggtcaccgtctcctca |
| 443 | light chain nuc | gccatccggttgacccaatctccatcctcactctctgcatctataggagacagagtcac |
| | | catcacttgtcgggcgagtcaggatattagcacttttttggcctggtatcaacaagagtca |
| | | ggtaaagccccaaggctcctaatctttgctgcatctactttacaaactggggtcccttca |
| | | aggttcagcggcagtggatctgggacagatttcactctcaccatcagcggcctgcaatc |
| | | tgaagattttgcaacttattactgtcaacagtattattgtttccctccggacttcggccaagg |
| | | gacacgactggacattaaa |

MGD33 ANTIBODY

| | | |
|---|---|---|
| 444 | CDRH1 aa | GGSITSSKW |
| 445 | CDRH2 aa | IYHNGTT |
| 446 | CDRH3 aa | ATASPFKSHHRTTEKLPRPSISAEPGTVIPLGSRVTFVCRGPVGV |
| | | QTFRLERETRSTYNDTEDVSQVSPSESEARFRIDSVSEGYAGPY |
| | | RCVYYKAPKWSEQSDYLDLLVKGEDVTWALTQPQLDPRACP |
| | | QGDLRMSTDIYCMDV |
| 447 | CDRL1 aa | QDISTY |
| 448 | CDRL2 aa | AAS |
| 449 | CDRL2 long aa | LIFAASSLQ |
| 450 | CDRL3 aa | QQYYCFPPD |
| 451 | CDRH1 nuc | ggtggctccatcaccagtagtaagtgg |
| 452 | CDRH2 nuc | atctatcataatgggaccacc |
| 453 | CDRH3 nuc | gcaacggcgtctcccttcaagtctcatcacaggaccaccgaaaaactgcccagacc |
| | | ctccatctcggctgagccgggcaccgtgatccccctggggagccgtgtgactttcgtgt |
| | | gccggggcccggttggggttcaaacattccgcctagagagggagactagatctacata |
| | | taatgatactgaagatgtgtctcaggttagtccgtctgagtcagaggccagattccgcatt |
| | | gactcagtgagtgagggatatgccgggccttatcgctgcgtctattataaggcccctaag |
| | | tggtccgagcagagtgactacctggacctgctggtgaaaggtgaggacgtcacttggg |
| | | ccctgacccagcctcagctcgaccctcgagcttgtccccagggggacctccgcatga |
| | | gcaccgatatttactgcatggacgtc |
| 454 | CDRL1 nuc | caggatattagcacttat |
| 455 | CDRL2 nuc | gctgcatct |
| 456 | CDRL2 long nuc | ctaatctttgctgcatctagtttacaa |
| 457 | CDRL3 nuc | caacaatattattgtttccctccggac |
| 458 | heavy chain aa | HVQLQESGPGLVKPSETLSLSCAVTGGSITSSKWWTWVRQGP |
| | | DKGLEWIGKIYHNGTTNYNPSLKSRVAMSVDKSKNQFSLRLT |
| | | SVTAADTAVYYCATASPFKSHHRTTEKLPRPSISAEPGTVIPLGS |
| | | RVTFVCRGPVGVQTFRLERETRSTYNDTEDVSQVSPSESEARFR |
| | | IDSVSEGYAGPYRCVYYKAPKWSEQSDYLDLLVKGEDVTWAL |
| | | TQPQLDPRACPQGDLRMSTDIYCMDWVGKGTTVTVSS |
| 459 | light chain aa | AIRLTQSPSSLSASIGDRVTITCRASQDISTYLAWYQQESGKAP |
| | | RLLIFAASSLQTGVPSRFSGSGSGTDFTLTISGLQSEDFATYYCQ |
| | | QYYCFPPDFGQGTRLDIK |
| 460 | heavy chain nuc | cacgtgcagctgcaggagtcgggcccaggactggtgaagccttcagaaaccctgtcc |
| | | ctctcctgcgctgtcactggtggctccatcaccagtagtaagtggtggacttgggtccgc |
| | | cagggcccagataaggggctggagtggattgggaaaatctatcataatgggaccacc |
| | | aactacaatccgtccctcaagagtcgagtcgccatgtcggtggacaagtccaagaac |
| | | cagttctccctgagactgacctccgtgaccgccgcggacacggccgtgtattactgtgc |
| | | aacggcgtctcccttcaagtctcatcacaggaccaccgaaaaactgcccagaccctc |
| | | catctcggctgagccgggcaccgtgatccccctggggagccgtgtgactttcgtgtgcc |
| | | ggggcccggttggggttcaaacattccgcctagagagggagactagatctacatataat |
| | | gatactgaagatgtgtctcaggttagtccgtctgagtcagaggccagattccgcattgac |
| | | tcagtgagtgagggatatgccgggccttatcgctgcgtctattataaggcccctaagtgg |
| | | tccgagcagagtgactacctggacctgctggtgaaaggtgaggacgtcacttgggccc |
| | | tgacccagcctcagctcgaccctcgagcttgtccccagggggacctccgcatgagca |
| | | ccgatatttactgcatggacgtctggggcaaagggaccacggtcaccgtctcctca |
| 461 | light chain nuc | gccatccggttgacccaatctccatcctcactctctgcatctataggagacagagtcac |
| | | catcacttgtcgggcgagtcaggatattagcacttatttggcctggtatcaacaagagtc |
| | | aggtaaagccccaaggctcctaatctttgctgcatctagtttacaaactggggtcccttc |
| | | aaggttcagcggcagtggatctgggacagatttcactctcaccatcagcggcctgcagt |
| | | ctgaagattttgcaacttattactgtcaacaatattattgtttccctccggacttcggccaag |
| | | ggacacgactggacattaaa |

MGD34 ANTIBODY

| | | |
|---|---|---|
| 462 | CDRH1 aa | GDYVNTNRR |
| 463 | CDRH2 aa | VHQSGRT |
| 464 | CDRH3 aa | ARASPLKSQRDTEDLPRPSISAEPGTVIPLGSHVTFVCRGPVGV |
| | | QTFRLERERNYLYSDTEDVSQTSPSESEARFRIDSVNAGNAGLF |
| | | RCIYYKSRKWSEQSDYLELWKGEDVTWALSQSQVDPRACP |
| | | QGELPISTDIYYVDV |
| 465 | CDRL1 aa | QHINDS |
| 466 | CDRL2 aa | GAS |
| 467 | CDRL2 long aa | LIYGASNLH |
| 468 | CDRL3 aa | QQCNCFPPD |
| 469 | CDRH1 nuc | ggtgactacgtcaatactaataggagg |
| 470 | CDRH2 nuc | gttcatcaaagtgggagaacc |
| 471 | CDRH3 nuc | gcgagagcgtctccactcaaatctcagagggacaccgaagatctgcccagaccctc |
| | | catctcggctgagccgggcaccgtgatccccctggggagccatgtgactttcgtgtgcc |
| | | ggggcccggttggggttcaaacattccgcctggagagggagaggaattatttatacagt |
| | | gatactgaagatgtgtctcaaactagtccatctgagtcggaggccagattccgcattgac |
| | | tcagtaaatgcaggcaatgccgggctttttcgctgcatctattacaagtcccgtaaatggt |
| | | ctgagcagagtgactacctggagctggtggtgaaaggtgaggacgtcacctgggccct |
| | | gtcccagtctcaaGTCGACcctcgagcttgtccccagggggagctccccataagt |
| | | accgatatttactacgtggacgtc |
| 472 | CDRL1 nuc | caacatattaatgattct |
| 473 | CDRL2 nuc | ggtgcatcc |
| 474 | CDRL2 long nuc | ctgatatatggtgcatccaatttgcac |
| 475 | CDRL3 nuc | caacagtgtaattgtttccctccggac |
| 476 | heavy chain aa | EVQLVESGPGLMKTSGTLSLTCAVSGDYVNTNRRWSWVRQA |
| | | PGKGLEWIGEVHQSGRTNYNPSLKSRVTISVDKSKNQFSLKV |
| | | DSVTAADTAVYYCARASPLKSQRDTEDLPRPSISAEPGTVIPLG |
| | | SHVTFVCRGPVGVQTFRLERERNYLYSDTEDVSQTSPSESEARF |
| | | RIDSVNAGNAGLFRCIYYKSRKWSEQSDYLELVVKGEDVTWA |
| | | LSQSQVDPRACPQGELPISTDIYYVDVWGNGTTFTVSS |
| 477 | light chain aa | AIRMTQSPSSLSASPGDKVSITCRASQHINDSLAWFQQRPGK |
| | | APKLLIYGASNLHSGVPSRFSGTGSGTDFTLTITGLQSEDFATY |
| | | FCQQCNCFPPDFGQGTRLEIK |
| 478 | heavy chain nuc | gaggtgcagctggtggagtcgggcccaggactgatgaagacttcggggaccctgtcc |
| | | ctcacgtgcgctgtgtctggtgactacgtcaatactaataggaggtggagttgggtccgc |
| | | caggccccagggaagggcctggagtggattggagaggttcatcaaagtgggagaac |
| | | caattacaacccgtccctcaagagccgagtcaccatatcagtagacaagtctaagaat |
| | | cagttctctctgaaggtggactctgtgaccgccgcggacacggccgtgtattactgtgcg |
| | | agagcgtctccactcaaatctcagagggacaccgaagatctgcccagaccctccatc |
| | | tcggctgagccgggcaccgtgatccccctggggagccatgtgactttcgtgtgccggg |
| | | gcccggttggggttcaaacattccgcctggagagggagaggaattatttatacagtgata |
| | | ctgaagatgtgtctcaaactagtccatctgagtcggaggccagattccgcattgactcag |
| | | taaatgcaggcaatgccgggctttttcgctgcatctattacaagtcccgtaaatggtctga |
| | | gcagagtgactacctggagctggtggtgaaaggtgaggacgtcacctgggccctgtcc |
| | | cagtctcaaGTCGACcctcgagcttgtccccagggggagctccccataagtaccg |
| | | atatttactacgtggacgtctggggcaacgggaccacgttcaccgtctcctca |
| 479 | light chain nuc | gccatccggatgacccagtctccatcctcactctctgcatcaccaggggacaaagtca |
| | | gcatcacttgtcgggcgagtcaacatattaatgattctttggcctggtttcaacaaaggcc |
| | | agggaaagccccaaaactcctgatatatggtgcatccaatttgcacagtggggtcccat |
| | | cgaggttcagcggcactgggtcagggacagatttcactctcactatcaccggcctgca |
| | | gtctgaagattttgcaacttatttctgtcaacagtgtaattgtttccctccggacttcggcca |
| | | agggacacgactggagattaaa |

MGD35 ANTIBODY

| | | |
|---|---|---|
| 480 | CDRH1 aa | GASISS1NW |
| 481 | CDRH2 aa | IHHNGST |
| 482 | CDRH3 aa | ATASSLKSQRDTNLPRPSLSAEPGTVIPLGSPVTFVCRGPVGVH |
| | | TFRLERAGRSTYNDTEDVSHPSPSESEARFRIDSVSEGNAGPYR |
| | | CVYYKSSKWSEESYCLDLLVKTEDVTWARPQPQLDPRACPQG |
| | | DLRISTDFYYMDV |
| 483 | CDRL1 aa | QAIGTY |
| 484 | CDRL2 aa | NAS |
| 485 | CDRL2 long aa | LIYNASTLQ |
| 486 | CDRL3 aa | QHYYNYPPA |
| 487 | CDRH1 nuc | ggtgcctccatcagtagtattaattgg |
| 488 | CDRH2 nuc | atccatcataatgggagcacc |
| 489 | CDRH3 nuc | gcgactgcctcttcattgaagtctcagagggacaccaatttgcccagaccctccctctc |
| | | ggcggagccaggcaccgtgatccccctggggagccctgtgactttcgtgtgccgggg |
| | | cccggttggggttcacacattccgcctggagagggcgggtagatccacatacaatgat |
| | | actgaagatgtgtctcatcctagtccatctgagtcagaggccagattccgcattgactca |
| | | gtgagtgagggaaatgccgggccttatcgctgcgtctattataagtcctctaaatggtcc |
| | | gaggagagttactgcctggacctgctggtcaaaactgaggacgtcacgtgggcccgg |
| | | ccccagcctcagctcgaccctcgagcttgtccccagggggacctccgcattagcacc |
| | | gatttttactacatggacgtc |
| 490 | CDRL1 nuc | caggctattggcacttat |
| 491 | CDRL2 nuc | aatgcttcc |
| 492 | CDRL2 long nuc | ctgatctataatgcttccactttgcaa |
| 493 | CDRL3 nuc | caacactattataattatcctccggcc |
| 494 | heavy chain aa | QVQLQESGPGLVKPSGTLSLTCAVSGASISSINWWSWVRQTP |
| | | EKGLEWIGQIHHNGSTNYNPSLKSRVAISVDKSKNQFSLKLTS |
| | | FTAADTAVYYCATASSLKSQRDTNLPRPSLSAEPGTVIPLGSPV |
| | | TFVCRGPVGVHTFRLERAGRSTYNDTEDVSHPSPSESEARFRID |
| | | SVSEGNAGPYRCVYYKSSKWSEESYCLDLLVKTEDVTVVARPQ |
| | | PQLDPRACPQGDLRISTDFYYMDVWGKGTTVTVSS |
| 495 | light chain aa | AIRMTQSPSSLSASTGDRVTITCRTSQAIGTYLAWYQQNPGK |
| | | APNLLIYNASTLQSGVPSRFSASGSGTDFTLTISGLQSDDFVTY |
| | | FCQHYYNYPPAFGQGTRLEIQ |
| 496 | heavy chain nuc | caggtgcagctgcaggagtcgggcccaggactggtgaagccttcggggaccctgtcc |
| | | ctcacctgcgctgtctctggtgcctccatcagtagtattaattggtggagttgggtccgtca |
| | | gaccccagaaaaggggctggagtggattggacaaatccatcataatgggagcacca |
| | | actacaacccgtccctcaagagtcgggtcgccatatcagttgacaagtccaagaacc |
| | | agttctccctgaagttgacttctttcaccgccgcggacacggccgtgtattattgtgcgac |
| | | tgcctcttcattgaagtctcagagggacaccaatttgcccagaccctccctctcggcgg |
| | | agccaggcaccgtgatccccctggggagccctgtgactttcgtgtgccggggcccggt |
| | | tggggttcacacattccgcctggagagggcgggtagatccacatacaatgatactgaa |
| | | gatgtgtctcatcctagtccatctgagtcagaggccagattccgcattgactcagtgagt |
| | | gagggaaatgccgggccttatcgctgcgtctattataagtcctctaaatggtccgaggag |
| | | agttactgcctggacctgctggtcaaaactgaggacgtcacgtgggcccggccccag |
| | | cctcagctcgaccctcgagcttgtccccagggggacctccgcattagcaccgattttta |
| | | ctacatggacgtctggggcaaagggaccacggtcaccgtctcttca |
| 497 | light chain nuc | gccatccggatgacccagtctccatcctcactctctgcatctacgggagacagagtca |
| | | ccatcacttgtcggacgagtcaggctattggcacttatttagcgtggtatcagcagaacc |
| | | cagggaaagcccctaacctcctgatctataatgcttccactttgcaaagtggggtcccat |
| | | caaggttcagcgccagtggctctgggacagatttcactctcaccatcagcggcctgca |
| | | gtctgacgattttgtcacttatttctgccaacactattataattatcctccggccttcggcca |
| | | agggacacgactggagattcaa |

MGD39 ANTIBODY

| | | |
|---|---|---|
| 498 | CDRH1 aa | GGSISAYRW |
| 499 | CDRH2 aa | VYNDGNT |
| 500 | CDRH3 aa | ATISPLRPQSDTDDLPRPSISAEPGTVIPLGSHVTFVCRGPIGVQ |
| | | TFRLERERRSLYSDTEDVSQVSPFASEARFRIDSVSEGNAGPYRC |
| | | IYYKDRKWSDQSDYLELLVKGEDVTWALPQSQLAPRACPQEE |
| | | LNISTDIFSMNV |
| 501 | CDRL1 aa | HDVGNY |
| 502 | CDRL2 aa | GAS |
| 503 | CDRL2 long aa | LIHGASTLQ |
| 504 | CDRL3 aa | QQYYSSPPG |
| 505 | CDRH1 nuc | ggtggctccatcagtgcttataggtgg |
| 506 | CDRH2 nuc | gtctataatgatggcaatacc |
| 507 | CDRH3 nuc | gcgacaatttctccactgaggcctcagagtgacaccgacgatctgcccagaccctcc |
| | | atctcggctgagccaggcaccgtgatccccctggggagccatgtgaccttcgtgtgcc |
| | | ggggcccaattggggttcaaacattccgcctggagagggagagaagatccttatacag |
| | | tgatactgaagatgtgtctcaagttagtccatttgcgtcagaggccagattccgcattgac |
| | | tcagtaagtgaaggaaatgccgggccatatcgctgcatctattataaggaccggaaatg |
| | | gtctgaccagagtgactacctggagttgctggtgaaaggtgaggacgtcacctgggcc |
| | | ctgccccagtctcagctcgcccctcgcgcttgtccccaggaagaattgaacattagtac |
| | | cgatattttctccatgaacgtc |
| 508 | CDRL1 nuc | catgatgttggtaattat |
| 509 | CDRL2 nuc | ggtgcgtcc |
| 510 | CDRL2 long nuc | ctgatccacggtgcgtccactttgcaa |
| 511 | CDRL3 nuc | caacaatattacagttcccctccgggc |
| 512 | heavy chain aa | QVRLQESGPGLVKPSGTLSLTCTVSGGSISAYRWWSWVRQA |
| | | PGKGLEWIGQVYNDGNTNYNPSLKGRVAMSVDKSKNRFSLR |
| | | LASVTAADTAVYYCATISPLRPQSDTDDLPRPSISAEPGTVIPLG |
| | | SHVTFVCRGPIGVQTFRLERERRSLYSDTEDVSQVSPFASEARF |
| | | RIDSVSEGNAGPYRCIYYKDRKWSDQSDYLELLVKGEDVTWA |
| | | LPQSQLAPRACPQEELNISTDIFSMNVWGKGTTVTVSS |
| 513 | light chain aa | AIRMTQSPASLSASIGDRVTITCRTSHDVGNYLDWYQQKPGK |
| | | APKLLIHGASTLQTGVPSRFSGSGAGTDFTLNITCLQSGDFAM |
| | | YYCQQYYSSPPGFGQGTRLEIK |
| 514 | heavy chain nuc | caggtgcggctgcaggagtcgggcccaggactggtgaagccacggggaccctgtcc |
| | | ctcacctgcactgtctctggtggctccatcagtgcttataggtggtggagttgggtccgcc |
| | | aggccccaggcaagggcctggagtggattggacaggtctataatgatggcaatacca |
| | | actacaacccgtccctcaagggtcgagtcgccatgtcagtggacaagtccaagaatc |
| | | gattttccctgagattagcgtctgtgaccgccgcggacacggccgtgtattactgtgcga |
| | | caatttctccactgaggcctcagagtgacaccgacgatctgcccagaccctccatctc |
| | | ggctgagccaggcaccgtgatccccctggggagccatgtgaccttcgtgtgccgggg |
| | | cccaattggggttcaaacattccgcctggagagggagagaagatccttatacagtgata |
| | | ctgaagatgtgtctcaagttagtccatttgcgtcagaggccagattccgcattgactcagt |
| | | aagtgaaggaaatgccgggccatatcgctgcatctattataaggaccggaaatggtctg |
| | | accagagtgactacctggagttgctggtgaaaggtgaggacgtcacctgggccctgcc |
| | | ccagtctcagctcgcccctcgcgcttgtccccaggaagaattgaacattagtaccgata |
| | | ttttctccatgaacgtctggggcaaagggaccacggtcaccgtctcctca |
| 515 | light chain nuc | gccatccggatgacccagtctccagcgtctctgtctgcatctataggagacagagtcac |
| | | catcacttgtcggacgagtcatgatgttggtaattatttagattggtatcaacaaaaacca |
| | | ggaaaagcccctaaactcctgatccacggtgcgtccactttgcaaactggggtcccat |
| | | cacggttcagcggcagtggagccgggacagatttcactctcaacatcacctgcctgca |
| | | gtctggagatttcgcaatgtattattgtcaacaatattacagttcccctccgggcttcggcc |
| | | aagggacacgactggagattaaa |

MGD41 ANTIBODY

| | | |
|---|---|---|
| 516 | CDRH1 aa | GGSINTDKW |
| 517 | CDRH2 aa | VLHTGST |
| 518 | CDRH3 aa | ATISTLRPQRDIEDLPRPSLSAEPGTVVPLGSHVTFVCRGPVGV |
| | | QTFRLERESRSTYNDTEDVSQPSPFESEARFRIDSVSEGNAGPY |
| | | RCIYYKSPKWSDQSDYVELLVKGEDVTWAPPQSQLAPRACP |
| | | QGELRTSTDIFSMNV |
| 519 | CDRL1 aa | QDIGNY |
| 520 | CDRL2 aa | GAS |
| 521 | CDRL2 long aa | LIHGASTLL |
| 522 | CDRL3 aa | LQYYSSPPA |
| 523 | CDRH1 nuc | ggtggctccatcaacactgataagtgg |
| 524 | CDRH2 nuc | gtccttcatactgggagcacc |
| 525 | CDRH3 nuc | gcgactatttctacattgaggcctcagcgggacatcgaagatctgcccagaccctccct |
| | | ctcggctgagccaggcaccgtggtccccctggggagccatgtgactttcgtgtgccgg |
| | | ggcccggttggggttcaaacattccgcctggagagggagagcagatccacatacaat |
| | | gatactgaagatgtgtctcaacctagtccatttgagtcagaggccagatttcgcattgact |
| | | cagtaagtgaaggaaatgccgggccttatcgctgcatctattataagtcccctaaatggt |
| | | ctgaccagagtgactacgtggagttgctggtgaaaggtgaggacgtcacctgggcccc |
| | | gccccagtctcagctcgcccctcgagcttgtccccagggagaactccgcactagcac |
| | | cgatattttctccatgaacgtc |
| 526 | CDRL1 nuc | caggatattggtaattac |
| 527 | CDRL2 nuc | ggtgcatcc |
| 528 | CDRL2 long nuc | ctgatccatggtgcatccactttgctg |
| 529 | CDRL3 nuc | ctacaatattacagttcccctccggcc |
| 530 | heavy chain aa | EVQLVESGPGLVKPSGTLSVTCTISGGSINTDKWWTWVRQPP |
| | | GKGLEWVGEVLHTGSTNYNPSLRGRVTISVDKSKNQFSLRLSS |
| | | VTAADTAVYYCATISTLRPQRDIEDLPRPSLSAEPGTVVPLGSH |
| | | VTFVCRGPVGVQTFRLERESRSTYNDTEDVSQPSPFESEARFRI |
| | | DSVSEGNAGPYRCIYYKSPKWSDQSDYVELLVKGEDVTWAPP |
| | | QSQLAPRACPQGELRTSTDIFSMNVWGKGTTVTVSS |
| 531 | light chain aa | AIRMTQSPSSLSAFTGDRVTISCRASQDIGNYLDWYHQKPGR |
| | | APKLLIHGASTLLTGVPSRFSGSGSGTDFTLNITCLQSGDFGIY |
| | | YCLQYYSSPPAFGPGTRLEIK |
| 532 | heavy chain nuc | gaggtgcagctggtggagtcgggcccaggactggtgaagccttcggggaccctgtcc |
| | | gtcacctgcactatctctggtggctccatcaacactgataagtggtggacttgggtccgc |
| | | cagcccccagggaagggccttgagtgggtaggggaagtccttcatactgggagcacc |
| | | aactacaacccgtccctgaggggtcgagtcaccatatcagtggacaagtccaagaac |
| | | cagttctccctgaggctgagttctgtgaccgccgcggacacggccgtatattattgtgcg |
| | | actatttctacattgaggcctcagcgggacatcgaagatctgcccagaccctccctctc |
| | | ggctgagccaggcaccgtggtccccctggggagccatgtgactttcgtgtgccggggc |
| | | ccggttggggttcaaacattccgcctggagagggagagcagatccacatacaatgata |
| | | ctgaagatgtgtctcaacctagtccatttgagtcagaggccagatttcgcattgactcagt |
| | | aagtgaaggaaatgccgggccttatcgctgcatctattataagtcccctaaatggtctga |
| | | ccagagtgactacgtggagttgctggtgaaaggtgaggacgtcacctgggccccgcc |
| | | ccagtctcagctcgcccctcgagcttgtccccagggagaactccgcactagcaccga |
| | | tattttctccatgaacgtctggggcaaagggaccacggtcaccgtctcctca |
| 533 | light chain nuc | gccatccggatgacccagtctccatcctcactgtctgcatttacaggagacagagtcac |
| | | catctcttgccgggcgagtcaggatattggtaattacttagattggtatcaccaaaagcc |
| | | aggaagagcccctaagctcctgatccatggtgcatccactttgctgactggggtcccat |
| | | cacgattcagcggcagtggatccggaacagatttcactctcaacatcacctgcctgca |
| | | gtctggagattttggaatttattactgtctacaatattacagttcccctccggccttcggccc |
| | | agggacacggctggagattaaga |

MGD47 ANTIBODY

| | | |
|---|---|---|
| 534 | CDRH1 aa | GGSISGYKW |
| 535 | CDRH2 aa | VYDDGDT |
| 536 | CDRH3 aa | ATISPLRPQSDTGDLPRPSISAEPGTAIPLGSQVTFVCRGPIGVQ |
| | | TFRLERESRALYNDSEDVSQVSPSASEARFRIDSVSEGNAGPYR |
| | | CIYYKARRWSDQSDYLELLVKGEDVTWALPQSQLAPRACPQE |
| | | DLNISTDIFSTNV |
| 537 | CDRL1 aa | QDVGNY |
| 538 | CDRL2 aa | GAS |
| 539 | CDRL2 long aa | LIHGASTLQ |
| 540 | CDRL3 aa | QQYYTSPPV |
| 541 | CDRH1 nuc | ggtggctccatcagtggttacaagtgg |
| 542 | CDRH2 nuc | gtctatgatgatggcgacacc |
| 543 | CDRH3 nuc | gcgacaatttctccactgaggcctcagagtgacaccggagatctgcccagaccctcc |
| | | atctcggctgagccaggcaccgcgatccccctggggagccaagtgactttcgtgtgcc |
| | | ggggcccaattggggttcaaacattccgcctggagagggagagtcgcgccttatataat |
| | | gattctgaagatgtgtctcaagttagtccatctgcgtcagaggccagattccgcattgact |
| | | cagtaagtgaaggcaatgccgggccttatcgctgtatctattataaggcccgcagatggt |
| | | ctgaccagagtgactatttggagttgttggtgaaaggtgaggacgtcacctgggccctgc |
| | | cccagtctcagctcgcccctcgcgcttgtccccaggaagatttgaacattagtaccgat |
| | | attttctctacgaacgtc |
| 544 | CDRL1 nuc | caggatgttggaaattat |
| 545 | CDRL2 nuc | ggtgcgtcc |
| 546 | CDRL2 long nuc | ctcatccacggtgcgtccactttgcaa |
| 547 | CDRL3 nuc | caacaatattacacttcccctccggtc |
| 548 | heavy chain aa | QVRLQESGPGLVKPSGTLSLTCTVSGGSISGYKWWSWVRQA |
| | | PGKGLEWIGQVYDDGDTNYNPDLKGRVALSVDKSKSRFSLSL |
| | | ASVTAADTAIYFCATISPLRPQSDTGDLPRPSISAEPGTAIPLGS |
| | | QVTFVCRGPIGVQTFRLERESRALYNDSEDVSQVSPSASEARFR |
| | | IDSVSEGNAGPYRCIYYKARRWSDQSDYLELLVKGEDVTWAL |
| | | PQSQLAPRACPQEDLNISTDIFSTNVWGKGTTVTVSS |
| 549 | light chain aa | AIRMTQSPASLSASVGDRVTITCRTSQDVGNYLDWYQQKPG |
| | | KAPKLLIHGASTLQAGVPSRFNGSGSGTDFTLGISCVQSGDFA |
| | | IYYCQQYYTSPPVFGQGTRLEIK |
| 550 | heavy chain nuc | caggtgcggctgcaggagtcgggcccaggactggtgaagccttcggggaccctgtcc |
| | | ctcacctgcactgtctcgggtggctccatcagtggttacaagtggtggagttgggtccgc |
| | | caggccccaggcaagggcctggagtggattggacaggtctatgatgatggcgacacc |
| | | aactacaatccggacctgaagggtcgagtcgccctgtcagtggacaagtccaagagt |
| | | cgattttccctcagcctagcgtctgtgaccgccgcggacacggccatatacttctgtgcg |
| | | acaatttctccactgaggcctcagagtgacaccggagatctgcccagaccctccatct |
| | | cggctgagccaggcaccgcgatccccctggggagccaagtgactttcgtgtgccggg |
| | | gcccaattggggttcaaacattccgcctggagagggagagtcgcgccttatataatgatt |
| | | ctgaagatgtgtctcaagttagtccatctgcgtcagaggccagattccgcattgactcag |
| | | taagtgaaggcaatgccgggccttatcgctgtatctattataaggcccgcagatggtctg |
| | | accagagtgactatttggagttgttggtgaaaggtgaggacgtcacctgggccctgccc |
| | | cagtctcagctcgcccctcgcgcttgtccccaggaagatttgaacattagtaccgatatt |
| | | ttctctacgaacgtctggggcaaagggacaacggtcaccgtctcttca |
| 551 | light chain nuc | gccatccggatgacccagtctccagcgtccctgtctgcatctgtaggagacagagtca |
| | | ccatcacttgtcggacgagtcaggatgttggaaattatttagattggtatcaacaaaaac |
| | | caggaaaagcccctaaactcctcatccacggtgcgtccactttgcaagctggggtccc |
| | | atcacgtttcaacggcagtggatccgggacagatttcactctcggcatcagttgtgtgca |
| | | gtctggagatttcgcgatctattactgtcaacaatattacacttcccctccggtcttccggcc |
| | | aagggacacgactggagattaaa |

MGD55 ANTIBODY

| | | |
|---|---|---|
| 552 | CDRH1 aa | GGSISAYKW |
| 553 | CDRH2 aa | VYHNGNT |
| 554 | CDRH3 aa | ATISPLRPQSDTDDLPRPSISAEPGTVIPLGSHVTFVCRGPIGVQ |
| | | TFRLERESRSLYSDTEDVSQVSPFASEARFRIDSVSEGNAGPYRC |
| | | IYYKDRKWSDQSDYLELLVKGEDVTWALPQSQLAPRACPQEE |
| | | LNISTDIFSMNV |
| 555 | CDRL1 aa | QDVGNY |
| 556 | CDRL2 aa | GAS |
| 557 | CDRL2 long aa | LIHGASTLQ |
| 558 | CDRL3 aa | QQYYSSPPG |
| 559 | CDRH1 nuc | ggtggctccatcagtgcttataagtgg |
| 560 | CDRH2 nuc | gtctatcataatggcaacacc |
| 561 | CDRH3 nuc | gcgacaatttctccactgaggcctcagagtgacaccgacgatctgcccagaccctcc |
| | | atctcggctgagccaggcaccgtgatccccctggggagccatgtgactttcgtgtgccg |
| | | gggcccaattggggttcaaacattccgcctggagagggagagtagatccttatacagtg |
| | | atactgaagatgtgtctcaagttagtccatttgcgtcagaggccagattccgcattgactc |
| | | agtaagtgaaggaaatgccgggccatatcgctgcatctattataaggaccggaaatggt |
| | | ctgaccagagtgactacctggagttgctggtgaaaggtgaggacgtcacctgggccct |
| | | gccccagtctcagctcgcccctcgcgcttgtccccaggaagaattgaacattagtacc |
| | | gatattttctccatgaacgtc |
| 562 | CDRL1 nuc | caggatgttggtaattat |
| 563 | CDRL2 nuc | ggtgcgtcc |
| 564 | CDRL2 long nuc | ctgatccacggtgcgtccactttgcaa |
| 565 | CDRL3 nuc | caacaatattacagttcccctccgggc |
| 566 | heavy chain aa | QVQLQESGPGLVKPSGTLSLTCTVSGGSISAYKWWSWVRQA |
| | | PGKGLEWIGQVYHNGNTNYNPSLKGRVAMSVDKSKNRFSLR |
| | | LASVTAADTAVYYCATISPLRPQSDTDDLPRPSISAEPGTVIPLG |
| | | SHVTFVCRGPIGVQTFRLERESRSLYSDTEDVSQVSPFASEARF |
| | | RIDSVSEGNAGPYRCIYYKDRKWSDQSDYLELLVKGEDVTWA |
| | | LPQSQLAPRACPQEELNISTDIFSMNVWGKGTTVTVSS |
| 567 | light chain aa | AIRMTQSPASLSASIGDRVTITCRTSQDVGNYLDWYQQKPGK |
| | | APKLLIHGASTLQTGVPSRFSGSGAGTDFTLNITCLQSGDFAM |
| | | YYCQQYYSSPPGFGQGTRLEIK |
| 568 | heavy chain nuc | caggtgcagctgcaggagtcgggcccaggactggtgaagccttcggggaccctgtcc |
| | | ctcacctgcactgtctctggtggctccatcagtgcttataagtggtggagttgggtccgcc |
| | | aggccccaggcaagggcctggagtggattggacaggtctatcataatggcaacacca |
| | | actacaacccgtccctcaagggtcgagtcgccatgtcagtggacaagtccaagaatc |
| | | gattttccctgagactagcgtctgtgaccgccgcggacacggccgtgtattactgtgcga |
| | | caatttctccactgaggcctcagagtgacaccgacgatctgcccagaccctccatctc |
| | | ggctgagccaggcaccgtgatccccctggggagccatgtgactttcgtgtgccggggc |
| | | ccaattggggttcaaacattccgcctggagagggagagtagatccttatacagtgatact |
| | | gaagatgtgtctcaagttagtccatttgcgtcagaggccagattccgcattgactcagta |
| | | agtgaaggaaatgccgggccatatcgctgcatctattataaggaccggaaatggtctga |
| | | ccagagtgactacctggagttgctggtgaaaggtgaggacgtcacctgggccctgccc |
| | | cagtctcagctcgcccctcgcgcttgtccccaggaagaattgaacattagtaccgatatt |
| | | ttctccatgaacgtctggggcaaagggaccacggtcaccgtctcctca |
| 569 | light chain nuc | gccatccggatgacccagtctccagcgtctctgtctgcatctataggagacagagtcac |
| | | catcacttgtcggacgagtcaggatgttggtaattatttagattggtatcaacaaaaacca |
| | | ggaaaagcccctaaactcctgatccacggtgcgtccactttgcaaactggggtcccat |
| | | cacggttcagcggcagtggagccgggacagatttcactctcaacatcacctgcctgca |
| | | gtctggagatttcgcaatgtattactgtcaacaatattacagttcccctccgggcttcggc |
| | | caagggacacgactggaaattaaga |

MGD56 ANTIBODY

| | | |
|---|---|---|
| 570 | CDRH1 aa | GGSITTNNW |
| 571 | CDRH2 aa | IFRSGTT |
| 572 | CDRH3 aa | ATASPFKSQRDTKDLPRPSLSAEPGTVIPLGSHVTFVCRGPVGV |
| | | QTFRLQRESRSLYNDTEDVSHPSPSESEARFRIDSVSEGNAGPY |
| | | RCVYYKSSKWSEESDCLELLVKTEDVTWARPQPQLDPRACPR |
| | | GDLRISTDVYYMDV |
| 573 | CDRL1 aa | QAITSY |
| 574 | CDRL2 aa | NAS |
| 575 | CDRL2 long aa | LIYNASTLQ |
| 576 | CDRL3 aa | QHYYTYPPA |
| 577 | CDRH1 nuc | ggtggctccatcactactaataattgg |
| 578 | CDRH2 nuc | atctttcgtagtgggaccacc |
| 579 | CDRH3 nuc | gcgacagcctctccattcaagtctcagagggacaccaaagatttgcccagaccctcc |
| | | ctctcggctgagccaggcaccgtgatccccctggggagtcatgtgactttcgtgtgccg |
| | | gggcccggttggggttcagacattccgcctgcagagggagagtagatccctttacaatg |
| | | atactgaagatgtgtctcatcctagtccatctgagtcagaggccagattccgcattgact |
| | | cagtgagtgagggaaatgccgggccttatcgctgcgtctattataagtcctctaaatggt |
| | | ccgaggagagtgactgcctggagctgctggtcaaaactgaggacgtcacctgggccc |
| | | ggccccagcctcagctcgaccctcgagcttgtccccggggggacctccgcattagca |
| | | ccgatgtttactacatggacgtc |
| 580 | CDRL1 nuc | caggctattaccagttat |
| 581 | CDRL2 nuc | aatgcttcc |
| 582 | CDRL2 long nuc | ctgatctataatgcttccactttgcaa |
| 583 | CDRL3 nuc | caacactattatacttaccctccggcc |
| 584 | heavy chain aa | QVQLQESGPGLVKPSGTLSLTCAVSGGSITTNNWWSWVRQT |
| | | PGKGLEWIGEIFRSGTTNYNPSLKSRVAISLDKSKNQFSLKLTSV |
| | | TAADTAVYYCATASPFKSQRDTKDLPRPSLSAEPGTVIPLGSHV |
| | | TFVCRGPVGVQTFRLQRESRSLYNDTEDVSHPSPSESEARFRID |
| | | SVSEGNAGPYRCVYYKSSKWSEESDCLELLVKTEDVTWARPQ |
| | | PQLDPRACPRGDLRISTDVYYMDVWGKGTTVTVSS |
| 585 | light chain aa | AIRMTQSPSSLSASTGDRVTITCRASQAITSYLAWYRQKPGKA |
| | | PDLLIYNASTLQSGVPSRFSASGSGTDFALTITGLQSEDFVIYFC |
| | | QHYYTYPPAFGQGTRLEIK |
| 586 | heavy chain nuc | caggtgcagctgcaggagtcgggcccaggactggtgaagccttcggggaccctgtcc |
| | | ctcacctgcgctgtctctggtggctccatcactactaataattggtggagttgggtccgtc |
| | | agaccccaggaaaggggctggagtggattggagaaatctttcgtagtgggaccacca |
| | | actacaacccgtccctcaagagtcgggtcgccatttcattagacaagtccaagaacca |
| | | gttctccctgaagttgacttctgtgaccgccgcggacacggccgtgtattactgtgcgac |
| | | agcctctccattcaagtctcagagggacaccaaagatttgcccagaccctccctctcg |
| | | gctgagccaggcaccgtgatccccctggggagtcatgtgactttcgtgtgccggggcc |
| | | cggttggggttcagacattccgcctgcagagggagagtagatccctttacaatgatactg |
| | | aagatgtgtctcatcctagtccatctgagtcagaggccagattccgcattgactcagtga |
| | | gtgagggaaatgccgggccttatcgctgcgtctattataagtcctctaaatggtccgagg |
| | | agagtgactgcctggagctgctggtcaaaactgaggacgtcacctgggcccggcccc |
| | | agcctcagctcgaccctcgagcttgtccccggggggacctccgcattagcaccgatgt |
| | | ttactacatggacgtctggggcaaagggaccacggtcaccgtctcctca |
| 587 | light chain nuc | gccatccggatgacccagtctccatcctcactctctgcatctacaggggacagagtca |
| | | ccatcacttgtcgggcgagtcaggctattaccagttatttagcctggtatcggcagaaac |
| | | cagggaaagcccctgacctcctgatctataatgcttccactttgcaaagtggggtcccat |
| | | caagattcagcgccagtggctagggacagatttcgctctcaccatcaccggcctgca |
| | | gtctgaggattttgtaatttatttctgccaacactattatacttaccctccggccttcggcca |
| | | agggacacgactggagattaaa |

Constant regions

| | | |
|---|---|---|
| 588 | IgG1 CH1—CH2—CH3 | ASTKGPSVFPLAPSSKSTSGGTAALGCLVKDYFPEPVTVSWNS |
| | aa | GALTSGVHTFPAVLQSSGLYSLSSVVTVPSSSLGTQTYICNVN |
| | | HKPSNTKVDKRVEPKSCDKTHTCPPCPAPELLGGPSVFLFPPKP |
| | | KDTLMISRTPEVTCVVVDVSHEDPEVKFNWYVDGVEVHNAK |
| | | TKPREEQYNSTYRVVSVLTVLHQDWLNGKEYKCKVSNKALPA |
| | | PIEKTISKAKGQPREPQVYTLPPSREEMTKNQVSLTCLVKGFYPS |
| | | DIAVEWESNGQPENNYKTTPPVLDSDGSFFLYSKLTVDKSRW |
| | | QQGNVFSCSVMHEALHNHYTQKSLSLSPGK |
| 589 | IgG CK aa | RTVAAPSVFIFPPSDEQLKSGTASVVCLLNNFYPREAKVQWKV |
| | | DNALQSGNSQESVTEQDSKDSTYSLSSTLTLSKADYEKHKVYA |
| | | CEVTHQGLSSPVTKSFNRGEC |
| 590 | IgG CL aa | GQPKAAPSVTLFPPSSEELQANKATLVCLISDFYPGAVTVAWK |
| | | ADSSPVKAGVETTTPSKQSNNKYAASSYLSLTPEQWKSHRSYS |
| | | CQVTHEGSTVEKTVAPTECS |
| 591 | IgG1 CH1—CH2—CH3 | gcgtcgaccaagggcccatcggtcttccccctggcaccctcctccaagagcacctct |
| | nucl | gggggcacagcggccctgggctgcctggtcaaggactacttccccgaacctgtgacg |
| | | gtctcgtggaactcaggcgccctgaccagcggcgtgcacaccttcccggctgtcctac |
| | | agtcctcaggactctactccctcagcagcgtggtgaccgtgccctccagcagcttggg |
| | | cacccagacctacatctgcaacgtgaatcacaagcccagcaacaccaaggtggaca |
| | | agagagttgagcccaaatcttgtgacaaaactcacacatgcccaccgtgcccagcac |
| | | ctgaactcctggggggaccgtcagtcttcctcttccccccaaaacccaaggacaccct |
| | | catgatctcccggacccctgaggtcacatgcgtggtggtggacgtgagccacgaAga |
| | | Ccctgaggtcaagttcaactggtacgtggacggcgtggaggtgcataatgccaagac |
| | | aaagccgcgggaggagcagtacaacagcacgtaccgtgtggtcagcgtcctcaccg |
| | | tcctgcaccaggactggctgaatggcaaggagtacaagtgcaaggtctccaacaaag |
| | | ccctcccagcccccatcgagaaaaccatctccaaagccaaagggcagccccgaga |
| | | accacaggtgtacaccctgcccccatcccgggaggagatgaccaagaaccaggtca |
| | | gcctgacctgcctggtcaaaggcttctatcccagcgacatcgccgtggagtgggagag |
| | | caatgggcagccggagaacaactacaagaccacgcctcccgtgctggactccgacg |
| | | gctccttcttcctctatagcaagctcaccgtggacaagagcaggtggcagcaggggaa |
| | | cgtcttctcatgctccgtgatgcatgaggctctgcacaaccactacacgcagaagagc |
| | | ctctccctgtccccgggtaaa |
| 592 | IgG CK nucl | cgTacGgtggctgcaccatctgtcttcatcacccgccatctgatgagcagttgaaatct |
| | | ggaactgcctctgttgtgtgcctgctgaataacttctatcccagagaggccaaagtacag |
| | | tggaaggtggataacgccctccaatcgggtaactcccaggagagtgtcacagagcag |
| | | gacagcaaggacagcacctacagcctcagcagcaccctgacgctgagcaaagcag |
| | | actacgagaaacacaaagtctacgcctgcgaagtcacccatcagggcctgagctcg |
| | | cccgtcacaaagagcttcaacaggggagagtgt |
| 593 | IgG CL nucl | ggtcagcccaaggctgccccctcggtcactctgttcccgccctcctctgaggagcttca |
| | | agccaacaaggccacactggtgtgtctcataagtgacttctacccgggagccgtgaca |
| | | gtggcttggaaagcagatagcagccccgtcaaggcgggagtggagaccaccacacc |
| | | ctccaaacaaagcaacaacaagtacgcggccagcagctatctgagcctgacgcctg |
| | | agcagtggaagtcccacagaagctacagctgccaggtcacgcatgaagggagcacc |
| | | gtggagaagacagtggcccctacagaatgttca |

It is also preferred that the protein according to the present invention, which is an antibody, is a recombinant antibody. As used herein, the term “recombinant antibody” is intended to include all antibodies that are prepared, expressed, created or isolated by recombinant means, such as antibodies isolated from a host cell such as for example a CHO cell or from an animal (e.g. a mouse) that is transgenic for human immunoglobulin genes or antibodies expressed using a recombinant expression vector transfected into a host cell. In particular, a “recombinant antibody” is not naturally occurring. Such recombinant antibodies may have variable and constant regions in a rearranged form. In particular, the term “recombinant antibody” includes various antibody formats, for example as described in FIG. 3 and the corresponding text of Carter, P. J. (2006) Nature Reviews immunology 6, 343-357. Preferably, a recombinant antibody is of an antibody format, which is not a naturally occurring antibody format. However, it is also preferred that the recombinant antibody is of a naturally occurring antibody format, preferably IgG, more preferably IgG1, but is not a naturally occurring antibody.

In particular, the term “recombinant antibody” includes “antibody fragments”, whereby the term “antibody fragment” refers to any fragment of an antibody of the invention that retains the specific binding activity of the antibody according to the invention, namely, the mutated LAIR-1 fragment as described herein, and, optionally, other components, whereby it is preferred that the recombinant antibody according to the present invention comprises in addition to the mutated LAIR-1 fragment as described herein an Fc moiety as described herein. Preferably, the recombinant antibody according to the present invention is of an IgG-based antibody format as described herein, more preferably a recombinant IgG-based antibody format, including antibody fragments as described herein.

Preferred examples of antibody fragments include, but are not limited to, a single chain antibody, Fab, Fab′, F(ab′)₂, Fv or scFv. Fragments of the antibodies of the invention can be obtained from the antibodies by methods that include digestion with enzymes, such as pepsin or papain, and/or by cleavage of disulfide bonds by chemical reduction. Alternatively, fragments of antibodies can be obtained by cloning and expression of part of the sequences of the heavy and/or light chains. “Fragments” include, but are not limited to, Fab, Fab′, F(ab′)2 and Fv fragments. The invention also encompasses single-chain Fv fragments (scFv) derived from the heavy and light chains of an antibody of the invention. For example, the invention includes a scFv comprising the LAIR-1 fragment according to the present invention. Also included are heavy or light chain monomers and dimers, single domain heavy chain antibodies, single domain light chain antibodies, as well as single chain antibodies, e.g., single chain Fv in which the heavy and light chain variable domains are joined by a peptide linker. Antibody fragments of the invention may impart monovalent or multivalent interactions and be contained in a variety of structures as described above. For instance, scFv molecules may be synthesized to create a trivalent “triabody” or a tetravalent “tetrabody.” The scFv molecules may include a domain of the Fc region resulting in bivalent minibodies. In addition, the sequences of the invention may be a component of multispecific molecules in which the sequences of the invention target the epitopes of the invention and other regions of the molecule bind to other targets. Although the specification, including the claims, may, in some places, refer explicitly to antigen binding fragment(s), antibody fragment(s), variant(s) and/or derivative(s) of antibodies, it is understood that the term “antibody” or “antibody of the invention” includes all categories of antibodies, namely, antigen binding fragment(s), antibody fragment(s), variant(s) and derivative(s) of antibodies. Further, the term “antibody” as used herein includes both antibodies and antigen binding fragments thereof.

The antibody according to the present invention is preferably a monospecific antibody, i.e. the antibody binds to one (single) epitope of an antigen. Such a monospecific antibody may be mono-, bi- or multivalent, i.e. the antibody has one, two or more antigen binding sites, which are all directed to the one (single) epitope of an antigen.

It is also preferred that the antibody according to the present invention is bi- or multispecific, i.e. the antibody binds to two or more epitopes of the same or different antigens. Preferably, the antibody comprises the mutated LAIR-1 fragment as described herein and thus binds to a RIFIN epitope as described herein, and the antibody additionally comprises one or more other malaria-specific binding sites. Such additional malaria-specific binding sites are preferably directed to erythrocytes infected with P. falciparum, more preferably to one or more epitopes of P. falciparum variant surface antigens (different from the RIFIN epitope as described herein to which the mutated LAIR-1 fragment binds to). Such a bi- or multispecific antibody may be bi- or multivalent. Exemplary bi- and multispecific antibodies include, but are not limited to, bispecific Fab2, trispecific Fab3, bispecific scFv, and diabodies (Holliger and Hudson, 2005, Nature Biotechnology 9: 1126-1136). Preferably, the antibody may be a multispecific antibody fragment with an Fc moiety. Examples, in particular for a bispecific antibody fragment with an Fc moiety, are Tandem scFv-Fc, scFv-Fc, scFv-Fc knobs-into-holes, scFv-Fc-scFv, and scDiabody-Fc, which are shown for example in FIG. 3b of Chan, A. C. and Carter, P. J. (2010) Nat Rev Immu 10: 301-316 and described in said article.

The (mono-, bi-, or multispecifc) antibody according to the present invention may preferably be based on any immunoglobulin class (e.g., IgA, IgG, IgM etc.) and subclass (e.g. IgA1, IgA2, IgG1, IgG2, IgG3, IgG4 etc.). Preferably, the antibody according to the present invention is based on IgG (also referred to as “IgG type”). Within the IgG class, antibodies may be based on the IgG1, IgG2, IgG3 or IgG4 subclass, whereby an antibody based on IgG1 (also referred to as “IgG1 type”) is preferred. Preferably, antibodies of the invention may have a κ or a λ light chain.

IgG-based antibody formats are well-known to the skilled person and preferred IgG-based antibody formats include for example hybrid hybridoma, knobs-into-holes with a common light chain, various IgG-scFv formats, various scFv-IgG formats, two-in-one IgG, dual (or multiple, respectively, e.g. 3 times, 4 times etc.) V domain IgG, IgG-V, and V-IgG, which are shown in FIG. 3c of Chan, A. C. and Carter, P. J. (2010) Nat Rev Immu 10: 301-316 and described in said article, for bispecific IgG-based antibodies, or any combination thereof resulting in a multispecific antibody of the IgG-type. Other preferred IgG-based antibody formats include for example DAF, CrossMab, IgG-dsscFv, DVD, IgG-dsFV, IgG-scFab, scFab-dsscFv, Fv2-Fc, Fab-scFV2, Fab-scFv, scFv-scFv BITE, diabodies, DART, TandAb and scFv-HAS-scFv which are shown in FIG. 1 of Weidle U. H. et al. (2013) Cancer Genomics and Proteomics 10: 1-18 and described in said article.

Preferably, the multispecific antibody, or the antigen binding fragment thereof, according to the present invention, is of the IgG type, preferably of the IgG1 type, more preferably comprising a heavy chain constant region of the IgG1 CH1-CH2-CH3 type and a light chain constant region of the IgG CK type or of the IgG CL type, even more preferably comprising (i) a heavy chain constant region of the IgG1 CH1-CH2-CH3 type comprising or consisting of an amino acid sequence according to SEQ ID NO: 588 or functional sequence variants thereof, and (ii) a light chain constant region of the IgG CK type comprising or consisting of an amino acid sequence according to SEQ ID NO: 589 or functional sequence variants thereof or a light chain constant region of the IgG CL type comprising or consisting of an amino acid sequence according to SEQ ID NO: 590 or functional sequence variants thereof.

As used herein, the term “constant domain” (also referred to as “constant region”) refers to a domain of an antibody which is not involved directly in binding an antibody to an antigen, but exhibits various effector functions. For example, antibodies or immunoglobulins may be divided in the classes: IgA, IgD, IgE, IgG and IgM, depending on the amino acid sequence of the constant region of their heavy chains. Several of these may be further divided into subclasses, e.g. IgG1, IgG2, IgG3, and IgG4, IgA1 and IgA2. The heavy chain constant regions that correspond to the different classes of immunoglobulins may be called α, ε, γ, and μ, respectively.

Preferably, the protein, in particular the antibody, according to the present invention comprises an Fc moiety. Preferably, the Fc moiety is derived from human origin, e.g. from human IgG1, IgG2, IgG3, and/or IgG4, whereby human IgG1 is particularly preferred.

As used herein, the term “Fc moiety” refers to a sequence derived from the portion of an immunoglobulin heavy chain beginning in the hinge region just upstream of the papain cleavage site (e.g., residue 216 in native IgG, taking the first residue of heavy chain constant region to be 114) and ending at the C-terminus of the immunoglobulin heavy chain. Accordingly, an Fc moiety may be a complete Fc moiety or a portion (e.g., a domain) thereof. A complete Fc moiety comprises at least a hinge domain, a CH2 domain, and a CH3 domain (e.g., EU amino acid positions 216-446). An additional lysine residue (K) is sometimes present at the extreme C-terminus of the Fc moiety, but is often cleaved from a mature antibody. Each of the amino acid positions within an Fc region have been numbered according to the art-recognized EU numbering system of Kabat, see e.g., by Kabat et al., in “Sequences of Proteins of Immunological Interest”, U.S. Dept. Health and Human Services, 1983 and 1987.

Preferably, in the context of the present invention an Fc moiety comprises at least one of: a hinge (e.g., upper, middle, and/or lower hinge region) domain, a CH2 domain, a CH3 domain, or a variant, portion, or fragment thereof. In preferred embodiments, an Fc moiety comprises at least a hinge domain, a CH2 domain or a CH3 domain. More preferably, the Fc moiety is a complete Fc moiety. The Fc moiety may also comprises one or more amino acid insertions, deletions, or substitutions relative to a naturally-occurring Fc moiety. For example, at least one of a hinge domain, CH2 domain or CI-13 domain (or portion thereof) may be deleted. For example, an Fc moiety may comprise or consist of: (i) hinge domain (or portion thereof) fused to a CH2 domain (or portion thereof), (ii) a hinge domain (or portion thereof) fused to a CH3 domain (or portion thereof), (iii) a CH2 domain (or portion thereof) fused to a CH3 domain (or portion thereof), (iv) a hinge domain (or portion thereof), (v) a CH2 domain (or portion thereof), or (vi) a CH3 domain or portion thereof.

It will be understood by one of ordinary skill in the art that the Fc moiety may be modified such that it varies in amino acid sequence from the complete Fc moiety of a naturally occurring immunoglobulin molecule, while retaining at least one desirable function conferred by the naturally-occurring Fc moiety. Such functions include Fc receptor (FcR) binding, antibody half-life modulation, ADCC function, protein A binding, protein G binding, and complement binding. The portions of naturally-occurring Fc moieties, which are responsible and/or essential for such functions are well known by those skilled in the art.

For example, to activate the complement cascade C1q binds to at least two molecules of IgG1 or one molecule of IgM, attached to the antigenic target (Ward, E. S., and Ghetie, V., Ther. Immunol. 2 (1995) 77-94). Burton, D. R., described (Mol. Immunol. 22 (1985) 161-206) that the heavy chain region comprising amino acid residues 318 to 337 is involved in complement fixation. Duncan, A. R., and Winter, G. (Nature 332 (1988) 738-740), using site directed mutagenesis, reported that Glu318, Lys320 and Lys322 form the binding site to C1q. The role of Glu318, Lys320 and Lys 322 residues in the binding of C1q was confirmed by the ability of a short synthetic peptide containing these residues to inhibit complement mediated lysis.

For example, FcR binding can be mediated by the interaction of the Fc moiety (of an antibody) with Fc receptors (FcRs), which are specialized cell surface receptors on hematopoietic cells. Fc receptors belong to the immunoglobulin superfamily, and were shown to mediate both the removal of antibody-coated pathogens by phagocytosis of immune complexes, and the lysis of erythrocytes and various other cellular targets (e.g. tumor cells) coated with the corresponding antibody, via antibody dependent cell mediated cytotoxicity (ADCC; Van de Winkel, J. G., and Anderson, C. L., J. Leukoc. Biol. 49 (1991) 511-524). FcRs are defined by their specificity for immunoglobulin classes; Fc receptors for IgG antibodies are referred to as FcγR, for IgE as FcER, for IgA as FcaR and so on and neonatal Fc receptors are referred to as FcRn. Fc receptor binding is described for example in Ravetch, J. V., and Kinet, J. P., Annu. Rev. Immunol. 9 (1991) 457-492; Capel, P. J., et al., Immunomethods 4 (1994) 25-34; de Haas, M., et al., J Lab. Clin. Med 126 (1995) 330-341; and Gessner, J. E., et al., Ann. Hematol. 76 (1998) 231-248.

Cross-linking of receptors by the Fc domain of native IgG antibodies (FcγR) triggers a wide variety of effector functions including phagocytosis, antibody-dependent cellular cytotoxicity, and release of inflammatory mediators, as well as immune complex clearance and regulation of antibody production. In humans, three classes of FcγR have been characterized, which are: (i) FcγRI (CD64), which binds monomeric IgG with high affinity and is expressed on macrophages, monocytes, neutrophils and eosinophils; (ii) FcγRII (CD32), which binds complexed IgG with medium to low affinity, is widely expressed, in particular on leukocytes, is known to be a central player in antibody-mediated immunity, and which can be divided into FcγRIIA, FcγRIIB and FcγRIIC, which perform different functions in the immune system, but bind with similar low affinity to the IgG-Fc, and the ectodomains of these receptors are highly homologuous; and (iii) FcγRIII (CD16), which binds IgG with medium to low affinity and exists as two types: FcγRIIIA found on NK cells, macrophages, eosinophils and some monocytes and T cells and mediating ADCC and FcγRIIIB, which is highly expressed on neutrophils. FcγRIIA is found on many cells involved in killing (e.g. macrophages, monocytes, neutrophils) and seems able to activate the killing process. FcγRIIB seems to play a role in inhibitory processes and is found on B-cells, macrophages and on mast cells and eosinophils. On B-cells it seems to function to suppress further immunoglobulin production and isotype switching to say for example the IgE class. On macrophages, FcγRIIB acts to inhibit phagocytosis as mediated through FcγRIIA. On eosinophils and mast cells the b form may help to suppress activation of these cells through IgE binding to its separate receptor.

Regarding FcγRI binding, modification in native IgG of at least one of E233-G236, P238, D265, N297, A327 and P329 reduces binding to FcγRI. IgG2 residues at positions 233-236, substituted into IgG1 and IgG4, reduces binding to FcγRI by 10³-fold and eliminated the human monocyte response to antibody-sensitized red blood cells (Armour, K. L., et al. Eur. J. Immunol. 29 (1999) 2613-2624). Regarding FcγRII binding, reduced binding for FcγRIIA is found e.g. for IgG mutation of at least one of E233-G236, P238, D265, N297, A327, P329, D270, Q295, A327, R292 and K414. Regarding FcγRIII binding, reduced binding to FcγRIIIA is found e.g. for mutation of at least one of E233-G236, P238, D265, N297, A327, P329, D270, Q295, A327, S239, E269, E293, Y296, V303, A327, K338 and D376. Mapping of the binding sites on human IgG1 for Fc receptors, the above mentioned mutation sites and methods for measuring binding to FcγRI and FcγRIIA are described in Shields, R. L., et al., J. Biol. Chem. 276 (2001) 6591-6604.

Regarding binding to the crucial FcγRII, two regions of native IgG Fc appear to be critical for interactions of FcγRIIs and IgGs, namely (i) the lower hinge site of IgG Fc, in particular amino acid residues L, L, G, G (234-237, EU numbering), and (ii) the adjacent region of the CH2 domain of IgG Fc, in particular a loop and strands in the upper CH2 domain adjacent to the lower hinge region, e.g. in a region of P331 (Wines, B. D., et al., J. Immunol. 2000; 164: 5313-5318). Moreover, FcγRI appears to bind to the same site on IgG Fc, whereas FcRn and Protein A bind to a different site on IgG Fc, which appears to be at the CH2-CH3 interface (Wines, B. D., et al., J. Immunol. 2000; 164: 5313-5318).

For example, the Fc moiety may comprise or consist of at least the portion of an Fc moiety that is known in the art to be required for FcRn binding or extended half-life. Alternatively or additionally, the Fc moiety of the antibody of the invention comprises at least the portion of known in the art to be required for Protein A binding and/or the Fc moiety of the antibody of the invention comprises at least the portion of an Fc molecule known in the art to be required for protein G binding. Preferably, the retained function is opsonizing of erythrocytes infected with P. falciparum, which is assumed to be mediated by FcγR binding. Accordingly, a preferred Fc moiety comprises at least the portion known in the art to be required for FcγR binding. As outlined above, a preferred Fc moiety may thus at least comprise (i) the lower hinge site of native IgG Fc, in particular amino acid residues L, L, G, G (234-237, EU numbering), and (ii) the adjacent region of the CH2 domain of native IgG Fc, in particular a loop and strands in the upper CH2 domain adjacent to the lower hinge region, e.g. in a region of P331, for example a region of at least 3, 4, 5, 6, 7, 8, 9, or 10 consecutive amino acids in the upper CH2 domain of native IgG Fc around P331, e.g. between amino acids 320 and 340 (EU numbering) of native IgG Fc.

Preferably, the protein, in particular the antibody, according to the present invention comprises an Fc region. As used herein, the term “Fc region” refers to the portion of an immunoglobulin formed by two or more Fc moieties of antibody heavy chains. For example, the Fc region may be monomeric or “single-chain” Fc region (i.e., a scFc region). Single chain Fc regions are comprised of Fc moieties linked within a single polypeptide chain (e.g., encoded in a single contiguous nucleic acid sequence). Exemplary scFc regions are disclosed in WO 2008/143954 A2. Preferably, the Fc region is a dimeric Fc region. A “dimeric Fc region” or “dcFc” refers to the dimer formed by the Fc moieties of two separate immunoglobulin heavy chains. The dimeric Fc region may be a homodimer of two identical Fc moieties (e.g., an Fc region of a naturally occurring immunoglobulin) or a heterodimer of two non-identical Fc moieties.

The Fc moieties of the Fc region may be of the same or different class and/or subclass. For example, the Fc moieties may be derived from an immunoglobulin (e.g., a human immunoglobulin) of an IgG1, IgG2, IgG3 or IgG4 subclass. Preferably, the Fc moieties of Fc region are of the same class and subclass. However, the Fc region (or one or more Fc moieties of an Fc region) may also be chimeric, whereby a chimeric Fc region may comprise Fc moieties derived from different immunoglobulin classes and/or subclasses. For example, at least two of the Fc moieties of a dimeric or single-chain Fc region may be from different immunoglobulin classes and/or subclasses. Additionally or alternatively, the chimeric Fc regions may comprise one or more chimeric Fc moieties. For example, the chimeric Fc region or moiety may comprise one or more portions derived from an immunoglobulin of a first subclass (e.g., an IgG1, IgG2, or IgG3 subclass) while the remainder of the Fc region or moiety is of a different subclass. For example, an Fc region or moiety of an Fc polypeptide may comprise a CH2 and/or CH3 domain derived from an immunoglobulin of a first subclass (e.g., an IgG1, IgG2 or IgG4 subclass) and a hinge region from an immunoglobulin of a second subclass (e.g., an IgG3 subclass). For example, the Fc region or moiety may comprise a hinge and/or CH2 domain derived from an immunoglobulin of a first subclass (e.g., an IgG4 subclass) and a CH3 domain from an immunoglobulin of a second subclass (e.g., an IgG1, IgG2, or IgG3 subclass). For example, the chimeric Fc region may comprise an Fc moiety (e.g., a complete Fc moiety) from an immunoglobulin for a first subclass (e.g., an IgG4 subclass) and an Fc moiety from an immunoglobulin of a second subclass (e.g., an IgG1, IgG2 or IgG3 subclass). For example, the Fc region or moiety may comprise a CH2 domain from an IgG4 immunoglobulin and a CH3 domain from an IgG1 immunoglobulin. For example, the Fc region or moiety may comprise a CH1 domain and a CH2 domain from an IgG4 molecule and a CH3 domain from an IgG1 molecule. For example, the Fc region or moiety may comprise a portion of a CH2 domain from a particular subclass of antibody, e.g., EU positions 292-340 of a CH2 domain. For example, an Fc region or moiety may comprise amino acids a positions 292-340 of CH2 derived from an IgG4 moiety and the remainder of CH2 derived from an IgG1 moiety (alternatively, 292-340 of CH2 may be derived from an IgG1 moiety and the remainder of CH2 derived from an IgG4 moiety).

Moreover, an Fc region or moiety may (additionally or alternatively) for example comprise a chimeric hinge region. For example, the chimeric hinge may be derived, e.g. in part, from an IgG1, IgG2, or IgG4 molecule (e.g., an upper and lower middle hinge sequence) and, in part, from an IgG3 molecule (e.g., an middle hinge sequence). In another example, an Fc region or moiety may comprise a chimeric hinge derived, in part, from an IgG1 molecule and, in part, from an IgG4 molecule. In another example, the chimeric hinge may comprise upper and lower hinge domains from an IgG4 molecule and a middle hinge domain from an IgG1 molecule. Such a chimeric hinge may be made, for example, by introducing a proline substitution (Ser228Pro) at EU position 228 in the middle hinge domain of an IgG4 hinge region. In another embodiment, the chimeric hinge can comprise amino acids at EU positions 233-236 are from an IgG2 antibody and/or the Ser228Pro mutation, wherein the remaining amino acids of the hinge are from an IgG4 antibody (e.g., a chimeric hinge of the sequence ESKYGPPCPPCPAPPVAGP). Further chimeric hinges, which may be used in the Fc moiety of the antibody according to the present invention are described in US 2005/0163783 A1.

Specifically included within the definition of “Fc region” is an “aglycosylated Fc region”. The term “aglycosylated Fc region” refers to an Fc region that lacks a covalently linked oligosaccharide or glycan, e.g., at the N-glycosylation site at EU position 297, in one or more of the Fc moieties thereof. For example, the aglycosylated Fc region may be fully aglycosylated, i.e., all of its Fc moieties lack carbohydrate. Alternatively, the aglycosylated Fc region may be partially aglycosylated (i.e., hemi-glycosylated). The aglycosylated Fc region may be a deglycosylated Fc region, that is an Fc region for which the Fc carbohydrate has been removed, for example chemically or enzymatically. Alternatively or additionally, the aglycosylated Fc region may be a nonglycosylated or unglycosylated, that is an antibody that was expressed without Fc carbohydrate, for example by mutation of one or residues that encode the glycosylation pattern, e.g., at the N-glycosylation site at EU position 297 or 299, by expression in an organism that does not naturally attach carbohydrates to proteins, (e.g., bacteria), or by expression in a host cell or organism whose glycosylation machinery has been rendered deficient by genetic manipulation or by the addition of glycosylation inhibitors (e.g., glycosyltransferase inhibitors). Alternatively, the Fc region is a “glycosylated Fc region”, i.e., it is fully glycosylated at all available glycosylation sites.

In the present invention it is preferred that the Fc moiety, or the Fc region, comprises or consists of an amino acid sequence derived from a human immunoglobulin sequence (e.g., from an Fc region or Fc moiety from a human IgG molecule). However, polypeptides may comprise one or more amino acids from another mammalian species. For example, a primate Fc moiety or a primate binding site may be included in the subject polypeptides. Alternatively, one or more murine amino acids may be present in the Fc moiety or in the Fc region.

Preferably, the protein, in particular the antibody, according to the present invention comprises, in particular in addition to an Fc moiety as described above, other parts derived from a constant region, in particular from a constant region of IgG, preferably from a constant region of IgG1, more preferably from a constant region of human IgG1. More preferably, the protein, in particular the antibody, according to the present invention comprises, in particular in addition to an Fc moiety as described above, all other parts of the constant regions, in particular all other parts of the constant regions of IgG, preferably all other parts of the constant regions of IgG1, more preferably all other parts of the constant regions of human IgG1.

As outlined above, a particularly preferred protein, in particular antibody, according to the present invention comprises a (complete) Fc region derived from human IgG1. More preferably, the multispecific antibody according to the present invention comprises, in particular in addition to a (complete) Fc region derived from human IgG1 also all other parts of the constant regions of IgG, preferably all other parts of the constant regions of IgG1, more preferably all other parts of the constant regions of human IgG1.

Preferred examples of recombinant antibodies comprising a mutated LAIR-1 fragment and an Fc moiety include, but are not limited to, the following constructs, which are described in detail—including their respective amino acid and nucleotide sequences—in Example 5 below: (i) “MGD21-DexinDJ-mIgG2b” (“M1”), (ii) “MGD21-exinDJ-mIgG2b” (“M2”), (iii) “MGD21-exin-mIgG2b” (“M3”), (iv) “MGD21-ex-mIgG2b” (“M4”), (v) “MGD21-DexinDJ-hIgG1” (“H1”), and (vi) “MGD21-ex-hIgG1” (“H2”). Thus, the recombinant antibody according to the present invention preferably comprises an amino acid sequence according to any of SEQ ID NO: 618, 624, 628, and 632 (cf. Table 10) or a functional sequence variant thereof. More preferably, the recombinant antibody according to the present invention comprises an amino acid sequence according to any of SEQ ID NO: 620, 622, 626, 630, 634 and 636 or a functional sequence variant thereof.

As described above, the Fc moiety enables the protein, in particular the antibody, according to the present invention to opsonize erythrocytes infected with P. falciparum and, thus, to limit P. falciparum infection. Thus, the antibody according to the present invention is preferably a neutralizing antibody. As used herein, a “neutralizing antibody” is an antibody that can neutralize, i.e., prevent, inhibit, reduce, impede or interfere with, the ability of a pathogen, in particular of P. falciparum, to initiate and/or perpetuate an infection in a host. These antibodies can be used alone, or in combination, as prophylactic or therapeutic agents upon appropriate formulation, in association with active vaccination, as a diagnostic tool, or as a production tool as described herein.

Production of Antibodies According to the Present Invention

Antibodies according to the present invention can be made by any method known in the art. For example, the general methodology for making monoclonal antibodies using hybridoma technology is well known (Kohler, G. and Milstein, C. 1975; Kozbar et al. 1983). In one embodiment, the alternative EBV immortalization method described in WO2004/076677 is used.

Using the method described in WO 2004/076677, B cells producing the antibody of the invention can be transformed with EBV and a polyclonal B cell activator. Additional stimulants of cellular growth and differentiation may optionally be added during the transformation step to further enhance the efficiency. These stimulants may be cytokines such as IL-2 and 1L-15. In one aspect, IL-2 is added during the immortalization step to further improve the efficiency of immortalization, but its use is not essential. The immortalized B cells produced using these methods can then be cultured using methods known in the art and antibodies isolated therefrom.

Using the method described in WO 2010/046775, plasma cells can be cultured in limited numbers, or as single plasma cells in microwell culture plates. Antibodies can be isolated from the plasma cell cultures. Further, from the plasma cell cultures, RNA can be extracted and PCR can be performed using methods known in the art. The VH and VL regions of the antibodies can be amplified by RT-PCR (reverse transcriptase PCR), sequenced and cloned into an expression vector that is then transfected into HEK293T cells or other host cells. The cloning of nucleic acid in expression vectors, the transfection of host cells, the culture of the transfected host cells and the isolation of the produced antibody can be done using any methods known to one of skill in the art.

Preferably, human monoclonal antibodies are prepared by using improved EBV-B cell immortalization as described in Traggiai E, Becker S, Subbarao K, Kolesnikova L, Uematsu Y, Gismondo M R, Murphy B R, Rappuoli R, Lanzavecchia A. (2004): An efficient method to make human monoclonal antibodies from memory B cells: potent neutralization of SARS coronavirus. Nat Med. 10(8):871-5.

The antibodies may be further purified, if desired, using filtration, centrifugation and various chromatographic methods such as HPLC or affinity chromatography. Techniques for purification of antibodies, e.g., monoclonal antibodies, including techniques for producing pharmaceutical-grade antibodies, are well known in the art.

Fragments of the antibodies of the invention can be obtained from the antibodies by methods that include digestion with enzymes, such as pepsin or papain, and/or by cleavage of disulfide bonds by chemical reduction. Alternatively, fragments of the antibodies can be obtained by cloning and expression of part of the sequences of the heavy or light chains. Antibody “fragments” include Fab, Fab′, F(ab′)2 and Fv fragments. The invention also encompasses single-chain Fv fragments (scFv) derived from the heavy and light chains of an antibody of the invention. For example, the invention includes a scFv comprising the CDRs from an antibody of the invention. Also included are heavy or light chain monomers and dimers, single domain heavy chain antibodies, single domain light chain antibodies, as well as single chain antibodies, e.g., single chain Fv in which the heavy and light chain variable domains are joined by a peptide linker. Antibody fragments of the invention may impart monovalent or multivalent interactions and be contained in a variety of structures as described above. For instance, scFv molecules may be synthesized to create a trivalent “triabody” or a tetravalent “tetrabody.” The scFv molecules may include a domain of the Fc region resulting in bivalent minibodies. In addition, the sequences of the invention may be a component of multispecific molecules in which the sequences of the invention target the epitopes of the invention and other regions of the molecule bind to other targets. Exemplary molecules include, but are not limited to, bispecific Fab2, trispecific Fab3, bispecific scFv, and diabodies (Holliger and Hudson, 2005, Nature Biotechnology 9: 1126-1136).

Standard techniques of molecular biology may be used to prepare DNA sequences encoding the antibodies or antibody fragments of the present invention. Desired DNA sequences may be synthesized completely or in part using oligonucleotide synthesis techniques. Site-directed mutagenesis and polymerase chain reaction (PCR) techniques may be used as appropriate.

Any suitable host cell/vector system may be used for expression of the DNA sequences encoding the antibody molecules of the present invention or fragments thereof. Bacterial, for example E. coli, and other microbial systems may be used, in part, for expression of antibody fragments such as Fab and F(ab′)2 fragments, and especially Fv fragments and single chain antibody fragments, for example, single chain Fvs. Eukaryotic, e.g., mammalian, host cell expression systems may be used for production of larger antibody molecules, including complete antibody molecules. Suitable mammalian host cells include, but are not limited to, CHO, HEK293T, PER.C6, NSO, myeloma or hybridoma cells.

The present invention also provides a process for the production of an antibody molecule according to the present invention comprising culturing a host cell comprising a vector encoding a nucleic acid of the present invention under conditions suitable for expression of protein from DNA encoding the antibody molecule of the present invention, and isolating the antibody molecule.

The antibody molecule may comprise only a heavy or light chain polypeptide, in which case only a heavy chain or light chain polypeptide coding sequence needs to be used to transfect the host cells. For production of products comprising both heavy and light chains, the cell line may be transfected with two vectors, a first vector encoding a light chain polypeptide and a second vector encoding a heavy chain polypeptide. Alternatively, a single vector may be used, the vector including sequences encoding light chain and heavy chain polypeptides.

Alternatively, antibodies according to the invention may be produced by (i) expressing a nucleic acid sequence according to the invention in a host cell, and (ii) isolating the expressed antibody product. Additionally, the method may include (iii) purifying the isolated antibody. Transformed B cells and cultured plasma cells may be screened for those producing antibodies of the desired specificity or function.

The screening step may be carried out by any immunoassay, e.g., ELISA, by staining of tissues or cells (including transfected cells), by neutralization assay or by one of a number of other methods known in the art for identifying desired specificity or function. The assay may select on the basis of simple recognition of one or more antigens, or may select on the additional basis of a desired function e.g., to select neutralizing antibodies rather than just antigen-binding antibodies, to select antibodies that can change characteristics of targeted cells, such as their signaling cascades, their shape, their growth rate, their capability of influencing other cells, their response to the influence by other cells or by other reagents or by a change in conditions, their differentiation status, etc.

Individual transformed B cell clones may then be produced from the positive transformed B cell culture. The cloning step for separating individual clones from the mixture of positive cells may be carried out using limiting dilution, micromanipulation, single cell deposition by cell sorting or another method known in the art.

Nucleic acid from the cultured plasma cells can be isolated, cloned and expressed in HEK293T cells or other known host cells using methods known in the art.

The immortalized B cell clones or the transfected host-cells of the invention can be used in various ways e.g., as a source of monoclonal antibodies, as a source of nucleic acid (DNA or mRNA) encoding a monoclonal antibody of interest, for research, etc.

The invention also provides a composition comprising immortalized B memory cells or transfected host cells that produce antibodies according to the present invention.

The immortalized B cell clone or the cultured plasma cells of the invention may also be used as a source of nucleic acid for the cloning of antibody genes for subsequent recombinant expression. Expression from recombinant sources is more common for pharmaceutical purposes than expression from B cells or hybridomas e.g., for reasons of stability, reproducibility, culture ease, etc.

Thus the invention also provides a method for preparing a recombinant cell, comprising the steps of: (i) obtaining one or more nucleic acids (e.g., heavy and/or light chain mRNAs) from the B cell clone or the cultured plasma cell that encodes the antibody of interest; (ii) inserting the nucleic acid into an expression vector and (iii) transfecting the vector into a host cell in order to permit expression of the antibody of interest in that host cell.

Similarly, the invention provides a method for preparing a recombinant cell, comprising the steps of: (i) sequencing nucleic acid(s) from the B cell clone or the cultured plasma cell that encodes the antibody of interest; and (ii) using the sequence information from step (i) to prepare nucleic acid(s) for insertion into a host cell in order to permit expression of the antibody of interest in that host cell. The nucleic acid may, but need not, be manipulated between steps (i) and (ii) to introduce restriction sites, to change codon usage, and/or to optimize transcription and/or translation regulatory sequences.

Furthermore, the invention also provides a method of preparing a transfected host cell, comprising the step of transfecting a host cell with one or more nucleic acids that encode an antibody of interest, wherein the nucleic acids are nucleic acids that were derived from an immortalized B cell clone or a cultured plasma cell of the invention. Thus the procedures for first preparing the nucleic acid(s) and then using it to transfect a host cell can be performed at different times by different people in different places (e.g., in different countries).

These recombinant cells of the invention can then be used for expression and culture purposes. They are particularly useful for expression of antibodies for large-scale pharmaceutical production. They can also be used as the active ingredient of a pharmaceutical composition. Any suitable culture technique can be used, including but not limited to static culture, roller bottle culture, ascites fluid, hollow-fiber type bioreactor cartridge, modular minifermenter, stirred tank, microcarrier culture, ceramic core perfusion, etc.

Methods for obtaining and sequencing immunoglobulin genes from B cells or plasma cells are well known in the art (e.g., see Chapter 4 of Kuby Immunology, 4th edition, 2000).

The transfected host cell may be a eukaryotic cell, including yeast and animal cells, particularly mammalian cells (e.g., CHO cells, NSO cells, human cells such as PER.C6 or HKB-11 cells, myeloma cells), as well as plant cells. Preferred expression hosts can glycosylate the antibody of the invention, particularly with carbohydrate structures that are not themselves immunogenic in humans. In one embodiment the transfected host cell may be able to grow in serum-free media. In a further embodiment the transfected host cell may be able to grow in culture without the presence of animal-derived products. The transfected host cell may also be cultured to give a cell line.

The present invention also provides a method for preparing one or more nucleic acid molecules (e.g., heavy and light chain genes) that encode an antibody of interest, comprising the steps of: (i) preparing an immortalized B cell clone or culturing plasma cells according to the invention; (ii) obtaining from the B cell clone or the cultured plasma cells nucleic acid that encodes the antibody of interest. Further, the invention provides a method for obtaining a nucleic acid sequence that encodes an antibody of interest, comprising the steps of: (i) preparing an immortalized B cell clone or culturing plasma cells according to the invention; (ii) sequencing nucleic acid from the B cell clone or the cultured plasma cell that encodes the antibody of interest.

The present invention further provides a method of preparing nucleic acid molecule(s) that encode an antibody of interest, comprising the step of obtaining the nucleic acid that was obtained from a transformed B cell clone or a cultured plasma cell of the invention. Thus the procedures for first obtaining the B cell clone or the cultured plasma cell, and then obtaining nucleic acid(s) from the B cell clone or the cultured plasma cell can be performed at different times by different people in different places (e.g., in different countries).

The present invention also comprises a method for preparing an antibody (e.g., for pharmaceutical use) according to the present invention, comprising the steps of: (i) obtaining and/or sequencing one or more nucleic acids (e.g., heavy and light chain genes) from the selected B cell clone or the cultured plasma cell expressing the antibody of interest; (ii) inserting the nucleic acid(s) into or using the nucleic acid(s) sequence(s) to prepare an expression vector; (iii) transfecting a host cell that can express the antibody of interest; (iv) culturing or sub-culturing the transfected host cells under conditions where the antibody of interest is expressed; and, optionally, (v) purifying the antibody of interest.

The present invention also provides a method of preparing an antibody comprising the steps of: culturing or sub-culturing a transfected host cell population under conditions where the antibody of interest is expressed and, optionally, purifying the antibody of interest, wherein said transfected host cell population has been prepared by (i) providing nucleic acid(s) encoding a selected antibody of interest that is produced by a B cell clone or cultured plasma cells prepared as described above, (ii) inserting the nucleic acid(s) into an expression vector, (iii) transfecting the vector in a host cell that can express the antibody of interest, and (iv) culturing or sub-culturing the transfected host cell comprising the inserted nucleic acids to produce the antibody of interest. Thus the procedures for first preparing the recombinant host cell and then culturing it to express antibody can be performed at very different times by different people in different places (e.g., in different countries).

Nucleic Acid Molecule According to the Present Invention

In another aspect, the present invention provides a nucleic acid molecule comprising a polynucleotide encoding a protein according to the present invention as described above.

Thus, nucleic acid molecules according to the present invention in particular encode a protein comprising or consisting of the mutated LAIR-1 fragment as described herein.

A nucleic acid molecule is a molecule comprising, preferably consisting of nucleic acid components. The term nucleic acid molecule preferably refers to DNA or RNA molecules. In particular, it is used synonymous with the term “polynucleotide”. Preferably, a nucleic acid molecule is a polymer comprising or consisting of nucleotide monomers which are covalently linked to each other by phosphodiester-bonds of a sugar/phosphate-backbone. The term “nucleic acid molecule” also encompasses modified nucleic acid molecules, such as base-modified, sugar-modified or backbone-modified etc. DNA or RNA molecules.

Nucleic acid molecules encoding a protein comprising or consisting of a mutated LAIR-1 fragment selected from the mutated LAIR-1 fragments according to SEQ ID NO: 10, 15, 16, 17, 18, 19, 20, 21 or 22, or a functional sequence variant thereof are preferred. Nucleic acid molecules encoding a protein comprising or consisting of an exemplary mutated LAIR-1 fragment selected from the exemplary mutated LAIR-1 fragments shown in Table 1, i.e. selected from the mutated LAIR-1 fragments according to SEQ ID NO: 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101 or 103, or a functional sequence variant thereof are more preferred. Thus, the nucleic acid molecule according to the present invention preferably comprises a polynucleotide sequence comprising or consisting of a nucleic acid sequence according to any one of SEQ ID NOs 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102 and 104 or a functional sequence variant thereof; more preferably the polynucleotide sequence comprises or consists of a nucleic acid sequence according to SEQ ID NO: 78, 84, 92, 96, 100 or 102; and even more preferably the polynucleotide sequence comprises or consists of a nucleic acid sequence according to SEQ ID NO: 84.

Particularly preferably, the nucleic acid molecules according to the present invention encode part or all of the light and heavy chains and CDRs of the exemplary antibodies of the present invention (cf. Tables 3 and 5). Preferably provided herein are thus nucleic acid sequences encoding part or all of the light and heavy chains, in particular VH and VL sequences and CDRs of the exemplary antibodies of the invention. The SEQ ID numbers for the nucleic acid sequences encoding the VH and VL sequences derived from monospecific antibodies and used in some examples of antibodies of the invention may be derived from Table 5. Table 6 below provides the SEQ ID numbers for the nucleic acid sequences encoding the CDRs of some examples of the antibodies of the invention. Due to the redundancy of the genetic code, the present invention also comprises variants of these nucleic acid sequences encoding the same amino acid sequences.

TABLE 6
SEQ ID Numbers for CDR polynucleotides derived from monospecific
antibodies as indicated and used in some exemplary antibodies
according to the present invention.

| | SEQ ID NOs. for CDR polynucleotides | | | | | |
|---|---|---|---|---|---|---|
| | CDRH1 | CDRH2 | CDRH3 | CDRL1 | CDRL2 | CDRL3 |
| MGC1 | 127 | 128 | 129 | 130 | 131/132 | 133 |
| MGC2 | 145 | 146 | 147 | 148 | 149/150 | 151 |
| MGC4 | 163 | 164 | 165 | 166 | 167/168 | 169 |
| MGC5 | 181 | 182 | 183 | 184 | 185/186 | 187 |
| MGC7 | 199 | 200 | 201 | 202 | 203/204 | 205 |
| MGC17 | 217 | 218 | 219 | 220 | 221/222 | 223 |
| MGC26 | 235 | 236 | 237 | 238 | 239/240 | 241 |
| MGC28 | 253 | 254 | 255 | 256 | 257/258 | 259 |
| MGC29 | 271 | 272 | 273 | 274 | 275/276 | 277 |
| MGC32 | 289 | 290 | 291 | 292 | 293/294 | 295 |
| MGC33 | 307 | 308 | 309 | 310 | 311/312 | 313 |
| MGC34 | 325 | 326 | 327 | 328 | 329/330 | 331 |
| MGC35 | 343 | 344 | 345 | 346 | 347/348 | 349 |
| MGC36 | 361 | 362 | 363 | 364 | 365/366 | 367 |
| MGC37 | 379 | 380 | 381 | 382 | 383/384 | 385 |
| MGD21 | 397 | 398 | 399 | 400 | 401/402 | 403 |
| MGD23 | 415 | 416 | 417 | 418 | 419/420 | 421 |
| MGD30 | 433 | 434 | 435 | 436 | 437/438 | 439 |
| MGD33 | 451 | 452 | 453 | 454 | 455/456 | 457 |
| MGD34 | 469 | 470 | 471 | 472 | 473/474 | 475 |
| MGD35 | 487 | 488 | 489 | 490 | 491/492 | 493 |
| MGD39 | 505 | 506 | 507 | 508 | 509/510 | 511 |
| MGD41 | 523 | 524 | 525 | 526 | 527/528 | 529 |
| MGD47 | 541 | 542 | 543 | 544 | 545/546 | 547 |
| MGD55 | 559 | 560 | 561 | 562 | 563/564 | 565 |
| MGD56 | 577 | 578 | 579 | 580 | 581/582 | 583 |

Preferably, the sequence of the nucleic acid molecule according to the present invention comprises or consists of a nucleic acid sequence according to any one of SEQ ID NOs: 127-133, 145-151, 163-169, 181-187, 199-205, 217-223, 235-241, 253-259, 271-277, 289-295, 307-313, 325-331, 343-349361-367, 379-385, 397-403, 415-421, 433-439, 451-457, 469-475, 487-493, 505-511, 523-529, 541-547, 559-565 and 577-583 or a functional sequence variant thereof. More preferably, the sequence of the nucleic acid molecule according to the present invention comprises or consists of a nucleic acid sequence according to any one of SEQ ID NOs: 129, 147, 165, 183, 291, 219, 237, 255, 273, 291, 309, 327, 345, 363, 381, 399, 417, 435, 453, 471, 489, 507, 525, 543, 561 and 579 or a functional sequence variant thereof.

It is also preferred that nucleic acid sequences according to the invention include nucleic acid sequences having at least 70%, at least 75%, at least 80%, at least 85%, at least 88%, at least 90%, at least 92%, at least 95%, at least 96%, at least 97%, at least 98% or at least 99% identity to the nucleic acid encoding a VH sequence and/or a VL sequence used in an antibody according to the present invention (cf. Table 4 above). Thus a nucleic acid molecule is preferred, wherein the polynucleotide sequence comprises or consists of a nucleic acid sequence according to any one of SEQ ID NOs: 136, 137, 154, 155, 172, 173, 190, 191, 208, 209, 226, 227, 244, 245, 262, 263, 280, 281, 298, 299, 316, 317, 334, 335, 352, 353, 370, 371, 388, 389, 406, 407, 424, 425, 460, 461, 478, 479, 496, 497, 514, 515, 532, 533, 550, 551, 568, 569, 586 and 587 or a functional sequence variant thereof. More preferably, a nucleic acid molecule according to the present invention comprises or consists of a nucleic acid sequence encoding a complete heavy chain or a complete light chain of one of the exemplary antibodies according to the present invention.

In general, the nucleic acid molecule according to the present invention may be manipulated to insert, delete or alter certain nucleic acid sequences. Changes from such manipulation include, but are not limited to, changes to introduce restriction sites, to amend codon usage, to add or optimize transcription and/or translation regulatory sequences, etc. It is also possible to change the nucleic acid to alter the encoded amino acids. For example, it may be useful to introduce one or more (e.g., 1, 2, 3, 4, 5, 6, 7, 8, 9, 10, etc.) amino acid substitutions, deletions and/or insertions into the antibody's amino acid sequence. Such point mutations can modify effector functions, antigen-binding affinity, post-translational modifications, immunogenicity, etc., can introduce amino acids for the attachment of covalent groups (e.g., labels) or can introduce tags (e.g., for purification purposes). Mutations can be introduced in specific sites or can be introduced at random, followed by selection (e.g., molecular evolution). For instance, one or more nucleic acids encoding any of the CDR regions, VH sequence or VL sequence, or a heavy or a light chain of an (exemplary) antibody of the invention can be randomly or directionally mutated to introduce different properties in the encoded amino acids. Such changes can be the result of an iterative process wherein initial changes are retained and new changes at other nucleotide positions are introduced. Further, changes achieved in independent steps may be combined. Different properties introduced into the encoded amino acids may include, but are not limited to, enhanced affinity.

Vector According to the Present Invention

In another aspect, the present invention provides a vector comprising the nucleic acid molecule according to the present invention, for example a nucleic acid molecule as described above. Such a vector according to the present invention is preferably a storage vector, an expression vector, a cloning vector, or a transfer vector, more preferably an expression vector or a cloning vector, and even more preferably an expression vector.

The term “vector” refers to a nucleic acid molecule, preferably to an artificial nucleic acid molecule, i.e. a nucleic acid molecule which does not occur in nature. A vector in the context of the present invention is suitable for incorporating or harboring a desired nucleic acid sequence. Such vectors may be storage vectors, expression vectors, cloning vectors, transfer vectors etc. A storage vector is a vector which allows the convenient storage of a nucleic acid molecule. Thus, the vector may comprise a sequence corresponding, e.g., to a desired antibody or antibody fragment thereof according to the present invention. An expression vector may be used for production of expression products such as RNA, e.g. mRNA, or peptides, polypeptides or proteins. For example, an expression vector may comprise sequences needed for transcription of a sequence stretch of the vector, such as a promoter sequence. A cloning vector is typically a vector that contains a cloning site, which may be used to incorporate nucleic acid sequences into the vector. A cloning vector may be, e.g., a plasmid vector or a bacteriophage vector. A transfer vector may be a vector which is suitable for transferring nucleic acid molecules into cells or organisms, for example, viral vectors. A vector in the context of the present invention may be, e.g., an RNA vector or a DNA vector. Preferably, a vector is a DNA molecule. For example, a vector in the sense of the present application comprises a cloning site, a selection marker, such as an antibiotic resistance factor, and a sequence suitable for multiplication of the vector, such as an origin of replication. Preferably, a vector in the context of the present application is a plasmid vector.

Cell According to the Present Invention

In another aspect, the present invention provides a cell expressing the protein according to the present invention or comprising the vector according to the present invention.

Thus, cells transformed with a vector according to the present invention are also included within the scope of the invention. Examples of such cells include but are not limited to, eukaryotic cells, e.g., yeast cells, animal cells or plant cells. In one embodiment the cells are mammalian, e.g., human, CHO, HEK293T, PER.C6, NS0, myeloma or hybridoma cells.

In particular, the cell may be transfected with a vector according to the present invention, preferably with an expression vector. The term “transfection” refers to the introduction of nucleic acid molecules, such as DNA or RNA (e.g. mRNA) molecules, into cells, preferably into eukaryotic cells. In the context of the present invention, the term “transfection” encompasses any method known to the skilled person for introducing nucleic acid molecules into cells, preferably into eukaryotic cells, such as into mammalian cells. Such methods encompass, for example, electroporation, lipofection, e.g. based on cationic lipids and/or liposomes, calcium phosphate precipitation, nanoparticle based transfection, virus based transfection, or transfection based on cationic polymers, such as DEAE-dextran or polyethylenimine etc. Preferably, the introduction is non-viral.

Pharmaceutical Composition According to the Present Invention

The present invention also provides a pharmaceutical composition comprising one or more of:
(i) the protein, preferably the antibody, more preferably the recombinant antibody, according to the present invention;
(ii) the nucleic acid molecule encoding the protein, preferably the antibody, more preferably the recombinant antibody, according to the present invention;
(iii) the vector encoding the nucleic acid molecule according to the present invention; or
(iv) the cell expressing the protein, preferably the antibody, more preferably the recombinant antibody, according to the present invention or comprising the vector according to the present invention.

Optionally, the pharmaceutical composition according to the present invention may also comprise one or more additional pharmaceutically active components and/or one or more pharmaceutically inactive components.

The pharmaceutical composition may also contain a pharmaceutically acceptable carrier, diluent and/or excipient. Preferably, the pharmaceutical composition according to the present invention comprises one or more of:
(i) the protein, preferably the antibody, more preferably the recombinant antibody, according to the present invention;
(ii) the nucleic acid molecule encoding the protein, preferably the antibody, more preferably the recombinant antibody, according to the present invention;
(iii) the vector encoding the nucleic acid molecule according to the present invention; or
(iv) the cell expressing the protein, preferably the antibody, more preferably the recombinant antibody, according to the present invention or comprising the vector according to the present invention; and

a pharmaceutically acceptable excipient, diluent and/or carrier.

Although the carrier or excipient may facilitate administration, it should not itself induce the production of antibodies harmful to the individual receiving the composition. Nor should it be toxic. Suitable carriers may be large, slowly metabolized macromolecules such as proteins, polypeptides, liposomes, polysaccharides, polylactic acids, polyglycolic acids, polymeric amino acids, amino acid copolymers and inactive virus particles.

Pharmaceutically acceptable salts can be used, for example mineral acid salts, such as hydrochlorides, hydrobromides, phosphates and sulphates, or salts of organic acids, such as acetates, propionates, malonates and benzoates.

Pharmaceutically acceptable carriers in therapeutic compositions may additionally contain liquids such as water, saline, glycerol and ethanol. Additionally, auxiliary substances, such as wetting or emulsifying agents or pH buffering substances, may be present in such compositions. Such carriers enable the pharmaceutical compositions to be formulated as tablets, pills, dragees, capsules, liquids, gels, syrups, slurries and suspensions, for ingestion by the subject.

Pharmaceutical compositions according to the present invention may be prepared in various forms. For example, the compositions may be prepared as injectables, either as liquid solutions or suspensions. Solid forms suitable for solution in, or suspension in, liquid vehicles prior to injection can also be prepared (e.g., a lyophilized composition, like Synagis™ and Herceptin™, for reconstitution with sterile water containing a preservative). The pharmaceutical composition may be prepared for topical administration e.g., as an ointment, cream or powder. The pharmaceutical composition may be prepared for oral administration e.g., as a tablet or capsule, as a spray, or as a syrup (optionally flavored). The pharmaceutical composition may be prepared for pulmonary administration e.g., as an inhaler, using a fine powder or a spray. The pharmaceutical composition may be prepared as a suppository or pessary. The pharmaceutical composition may be prepared for nasal, aural or ocular administration e.g., as drops. The pharmaceutical composition may be in kit form, designed such that a combined composition is reconstituted just prior to administration to a subject. For example, a lyophilized antibody can be provided in kit form with sterile water or a sterile buffer.

It is preferred that the active ingredient in the composition is the protein, preferably the antibody, more preferably the recombinant antibody, according to the present invention. As such, it may be susceptible to degradation in the gastrointestinal tract. Thus, if the composition is to be administered by a route using the gastrointestinal tract, the composition may contain agents which protect the protein, preferably the antibody, more preferably the recombinant antibody, according to the present invention from degradation but which release the protein once it has been absorbed from the gastrointestinal tract.

A thorough discussion of pharmaceutically acceptable carriers is available in Gennaro (2000) Remington: The Science and Practice of Pharmacy, 20th edition, ISBN: 0683306472.

Pharmaceutical compositions of the invention generally have a pH in particular between 5.5 and 8.5, for example between 6 and 8, for example about 7. The pH may be maintained by the use of a buffer. The pharmaceutical composition may be sterile and/or pyrogen free. The pharmaceutical composition may be isotonic with respect to humans. The pharmaceutical composition of the invention may be supplied in hermetically-sealed containers.

Within the scope of the invention are compositions present in several forms for different administration methods; the forms include, but are not limited to, those forms suitable for parenteral administration, e.g., by injection or infusion, for example by bolus injection or continuous infusion. Where the product is for injection or infusion, it may take the form of a suspension, solution or emulsion in an oily or aqueous vehicle and it may contain formulatory agents, such as suspending, preservative, stabilizing and/or dispersing agents. Alternatively, the protein may be in dry form, for reconstitution before use with an appropriate sterile liquid. A vehicle is typically understood to be a material that is suitable for storing, transporting, and/or administering a compound, such as a pharmaceutically active compound, in particular the protein according to the present invention. For example, the vehicle may be a physiologically acceptable liquid, which is suitable for storing, transporting, and/or administering a pharmaceutically active compound, in particular the antibodies according to the present invention. Once formulated, the pharmaceutical composition according to the present invention may be administered directly to the subject. In one embodiment the pharmaceutical composition according to the present invention is adapted for administration to mammalian, e.g., human subjects.

The pharmaceutical composition according to the present invention may be administered by any number of routes including, but not limited to, oral, intravenous, intramuscular, intra-arterial, intramedullary, intraperitoneal, intrathecal, intraventricular, transdermal, transcutaneous, topical, subcutaneous, intranasal, enteral, sublingual, intravaginal or rectal routes. Hyposprays may also be used to administer the pharmaceutical composition according to the present invention. Preferably, the pharmaceutical composition according to the present invention may be prepared for oral administration, e.g. as tablets, capsules and the like, for topical administration, or as injectable, e.g. as liquid solutions or suspensions. Solid forms suitable for solution in, or suspension in, liquid vehicles prior to injection may also be prepared.

For injection, e.g. intravenous, cutaneous or subcutaneous injection, or injection at the site of affliction, the active ingredient will preferably be in the form of a parenterally acceptable aqueous solution which is pyrogen-free and has suitable pH, isotonicity and stability. Those of relevant skill in the art are well able to prepare suitable solutions using, for example, isotonic vehicles such as Sodium Chloride Injection, Ringer's Injection, Lactated Ringer's Injection.

Preferably, preservatives, stabilizers, buffers, antioxidants and/or other additives may be included in the pharmaceutical composition according to the present invention, as required.

Whether it is a protein, a nucleic acid molecule, or a cell according to the present invention that is to be given to an individual by administering the pharmaceutical composition according to the present invention, administration is preferably in a “prophylactically effective amount” (of the protein, the nucleic acid molecule, or the cell according to the present invention) or a “therapeutically effective amount” (of the protein, the nucleic acid molecule, or the cell according to the present invention) (as the case may be), this being sufficient to show benefit to the individual. The actual amount administered, and rate and time-course of administration, will depend on the nature and severity of what is being treated. For injection, the pharmaceutical composition according to the present invention may be provided for example in a pre-filled syringe.

The pharmaceutical composition according to the present invention may also be administered orally in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient, i.e. the protein according to the present invention as defined above, is combined with emulsifying and suspending agents. If desired, certain sweetening, flavoring or coloring agents may also be added.

The inventive pharmaceutical composition may also be administered topically. For topical applications, the pharmaceutical composition according to the present invention may be formulated in a suitable ointment, containing the pharmaceutical composition, particularly its components as defined above, suspended or dissolved in one or more carriers. Carriers for topical administration include, but are not limited to, mineral oil, liquid petrolatum, white petrolatum, propylene glycol, polyoxyethylene, polyoxypropylene compound, emulsifying wax and water. Alternatively, the pharmaceutical composition according to the present invention may be formulated in a suitable lotion or cream. In the context of the present invention, suitable carriers include, but are not limited to, mineral oil, sorbitan monostearate, polysorbate 60, cetyl esters wax, cetearyl alcohol, 2-octyldodecanol, benzyl alcohol and water.

Dosage treatment may be a single dose schedule or a multiple dose schedule, whereby in the context of the present invention a multiple dose schedule is preferred. Known antibody-based pharmaceuticals, in particular anti-Malaria-antibody based pharmaceuticals, provide guidance relating to frequency of administration e.g., whether a pharmaceutical should be delivered daily, weekly, monthly, etc. Frequency and dosage may also depend on the severity of symptoms.

For example, the pharmaceutical composition according to the present invention may be administered daily, e.g. once or several times per day, e.g. once, twice, three times or four times per day, preferably once or twice per day, more preferable once per day, for 1, 2, 3, 4, 5, 6, 7, 8, 9, 10, 11, 12, 13, 14, 15, 16, 17, 18, 19, 20 or 21 or more days, e.g. daily for 1, 2, 3, 4, 5, 6 months. Preferably, the pharmaceutical composition according to the present invention may be administered weekly, e.g. once or twice, preferably once per week, for 1, 2, 3, 4, 5, 6, 7, 8, 9, 10, 11, 12, 13, 14, 15, 16, 17, 18, 19, 20 or 21 or more weeks, e.g. weekly 1, 2, 3, 4, 5, 6, 7, 8, 9, 10, 11, or 12 months or weekly for 2, 3, 4, or 5 years.

In particular, it is preferred that for a single dose, e.g. a daily, weekly or monthly dose, preferably for a weekly dose, the amount of the protein, preferably of the antibody, more preferably of the recombinant antibody, according to the present invention, in the pharmaceutical composition according to the present invention, does not exceed 150 mg, preferably does not exceed 100 mg, more preferably does not exceed 50 mg, even more preferably does not exceed 20 mg, and particularly preferably does not exceed 10 mg. This amount of protein/antibody preferably refers to a single dose as described above, which is for example administered daily, weekly etc. as described above. Such a low amount of the protein/antibody according to the present invention could be produced and formulated in a stable form (e.g., in a lyophilized formulation, where for instance previous studies have shown that monoclonal antibodies preserved by lyophilization are stable for 33 months at 40° C. and 5 months at 50° C.) and at an affordable cost.

Pharmaceutical compositions typically include an effective amount of one or more proteins, preferably antibodies, more preferably recombinant antibodies, of the invention, i.e. an amount that is sufficient to treat, ameliorate, attenuate or prevent a desired disease or condition, or to exhibit a detectable therapeutic effect. Therapeutic effects also include reduction or attenuation in pathogenic potency or physical symptoms. The precise effective amount for any particular subject will depend upon their size, weight, and health, the nature and extent of the condition, and the therapeutics or combination of therapeutics selected for administration. The effective amount for a given situation is determined by routine experimentation and is within the judgment of a clinician. For purposes of the present invention, an effective dose will generally be from about 0.005 to about 100 mg/kg, preferably from about 0.0075 to about 50 mg/kg, more preferably from about 0.01 to about 10 mg/kg, even more preferably from about 0.02 to about 5 mg/kg, and particularly preferably from about 0.03 to about 1 mg/kg of the antibody of the present invention (e.g. amount of the antibody in the pharmaceutical composition) in relation to the bodyweight (e.g., in kg) of the individual to which it is administered.

Preferably, the pharmaceutical composition according to the present invention may include two or more (e.g., 2, 3, 4, 5 etc.) proteins, preferably antibodies, more preferably recombinant antibodies, of the invention to provide an additive or synergistic therapeutic effect. The term “synergy” is used to describe a combined effect of two or more active agents that is greater than the sum of the individual effects of each respective active agent. Thus, where the combined effect of two or more agents results in “synergistic inhibition” of an activity or process, it is intended that the inhibition of the activity or process is greater than the sum of the inhibitory effects of each respective active agent. The term “synergistic therapeutic effect” refers to a therapeutic effect observed with a combination of two or more therapies wherein the therapeutic effect (as measured by any of a number of parameters) is greater than the sum of the individual therapeutic effects observed with the respective individual therapies.

It is also preferred that the pharmaceutical composition according to the present invention may comprise one or more (e.g., 2, 3, etc.) antibodies according the invention and one or more (e.g., 2, 3, etc.) additional antibodies, preferably against malaria, more preferably against P. falciparum, even more preferably against a variant surface antigen of P. falciparum, and particularly preferably against a P. falciparum RIFIN. Further, the administration of proteins, in particular antibodies, of the invention together with antibodies specific to other antigens, are within the scope of the invention. The antibodies of the invention can be administered either combined/simultaneously or at separate times from antibodies specific to other cytokines or, more generally, to other antigens.

In one embodiment, a composition of the invention may include proteins, preferably antibodies, of the invention, wherein the proteins/antibodies according to the present invention may make up at least 50% by weight (e.g., 60%, 70%, 75%, 80%, 85%, 90%, 95%, 97%, 98%, 99% or more) of the total protein in the pharmaceutical composition. In such a pharmaceutical composition, the proteins, preferably the antibodies, are preferably in purified form.

The present invention also provides a method of preparing a pharmaceutical composition comprising the steps of: (i) preparing a protein, preferably an antibody, according to the present invention; and (ii) admixing the optionally purified protein, preferably antibody, with one or more pharmaceutically-acceptable carriers.

In another embodiment, a method of preparing a pharmaceutical composition comprises the step of: admixing a protein, preferably an antibody, according to the present invention with one or more pharmaceutically-acceptable carriers, wherein the protein is a monoclonal antibody that was obtained from a transformed B cell or a cultured plasma cell of the invention. Thus the procedures for first obtaining the monoclonal antibody and then preparing the pharmaceutical can be performed at very different times by different people in different places (e.g., in different countries).

As an alternative to delivering antibodies or B cells for therapeutic purposes, it is possible to deliver a nucleic acid molecule, preferably a DNA molecule, that encodes the protein, preferably the antibody, according to the present invention derived from the B cell or the cultured plasma cells to a subject, such that the nucleic acid molecule can be expressed in the subject in situ to provide a desired therapeutic effect. Suitable gene therapy and nucleic acid delivery vectors are known in the art.

The pharmaceutical composition according to the present invention may include an antimicrobial, particularly if packaged in a multiple dose format. They may comprise detergent e.g., a Tween (polysorbate), such as Tween 80. Detergents are generally present at low levels e.g., less than 0.01%. The pharmaceutical composition according to the present invention may also include a sodium salt (e.g., sodium chloride) to give tonicity. For example, a concentration of 10±2mg/ml NaCl is typical.

Further, the pharmaceutical composition according to the present invention may comprise a sugar alcohol (e.g., mannitol) or a disaccharide (e.g., sucrose or trehalose) e.g., at around 15-30 mg/ml (e.g., 25 mg/ml), particularly if they are to be lyophilized or if they include material which has been reconstituted from lyophilized material. The pH of a composition for lyophilisation may be adjusted to between 5 and 8, or between 5.5 and 7, or around 6.1 prior to lyophilisation.

The pharmaceutical composition according to the present invention may also comprise one or more immunoregulatory agents. One or more of the immunoregulatory agents may include an adjuvant.

Medical Treatments and Uses

In a further aspect, the present invention provides the use of
(i) the protein, preferably the antibody, more preferably the recombinant antibody, according to the present invention;
(ii) the nucleic acid molecule encoding the protein, preferably the antibody, more preferably the recombinant antibody, according to the present invention;
(iii) the vector encoding the nucleic acid molecule according to the present invention;
(iv) the cell expressing the protein, preferably the antibody, more preferably the recombinant antibody, according to the present invention or comprising the vector according to the present invention; or
(v) the pharmaceutical composition according to the present invention as described herein

in prevention and/or treatment of malaria, preferably of P. falciparum-malaria.

Malaria is caused by Plasmodium parasites. The parasites are spread to people through the bites of infected Anopheles mosquitoes, called “malaria vectors”, which bite mainly between dusk and dawn. There are four parasite species that cause malaria in humans Plasmodium falciparum, Plasmodium vivax, Plasmodium malariae, and Plasmodium ovale. Plasmodium falciparum and Plasmodium vivax are the most common causes of malaria. Plasmodium falciparum is the most deadly.

Within the scope of the invention are several forms and routes of administration of the protein, preferably the antibody, the nucleic acid, the vector, the cell, or the pharmaceutical composition, as described above in respect to the pharmaceutical composition. This applies also in the context of the use of the protein, the nucleic acid, the vector, the cell as described herein, in particular regarding preferred forms and routes of administration.

In a further aspect, the present invention provides the use of
(i) the protein, preferably the antibody, more preferably the recombinant antibody, according to the present invention;
(ii) the nucleic acid molecule encoding the protein, preferably the antibody, more preferably the recombinant antibody, according to the present invention;
(iii) the vector encoding the nucleic acid molecule according to the present invention;
(iv) the cell expressing the protein, preferably the antibody, more preferably the recombinant antibody, according to the present invention or comprising the vector according to the present invention; or
(v) the pharmaceutical composition according to the present invention as described herein

in diagnosis of malaria, preferably of P. falciparum-malaria.

Methods of diagnosis may include contacting a protein, preferably an antibody, according to the present invention with a sample. Such samples may be isolated from a subject, for example an isolated tissue sample taken from, for example, nasal passages, sinus cavities, salivary glands, lung, liver, pancreas, kidney, ear, eye, placenta, alimentary tract, heart, ovaries, pituitary, adrenals, thyroid, brain, skin or blood, preferably serum.

In the context of the present invention, diagnosis of malaria is preferably done by contacting a protein, preferably an antibody, according to the present invention with a sample, which is preferably isolated, e.g. from a patient. The sample is preferably a (isolated) sample comprising erythrocytes, more preferably a blood sample, more preferably a (isolated) sample of blood fragment(s) comprising erythrocytes.

The methods of diagnosis may also include the detection of an antigen/protein complex, e.g. an antigen/antibody complex, in particular following the contacting of a protein with a sample. Such a detection step is typically performed at the bench, i.e. without any contact to the human or animal body. Examples of detection methods include e.g. ELISA (enzyme-linked immunosorbent assay).

Diagnosis of malaria, e.g. in a blood sample, is important for example (i) for a subject, which may potentially suffer from malaria, and (ii) for blood transfusions to avoid transmission of malaria by infected blood transfusions. In particular in this context the protein according to the present invention, which binds broadly to different strains of P. falciparum may be very useful to determine whether a blood sample is malaria-free.

Thus, the present invention provides the use of
(i) the protein, preferably the antibody, more preferably the recombinant antibody, according to the present invention;
(ii) the nucleic acid molecule encoding the protein, preferably the antibody, more preferably the recombinant antibody, according to the present invention;
(iii) the vector encoding the nucleic acid molecule according to the present invention;
(iv) the cell expressing the protein, preferably the antibody, more preferably the recombinant antibody, according to the present invention or comprising the vector according to the present invention; or
(v) the pharmaceutical composition according to the present invention as described herein

in determining whether a blood sample, preferably an isolated blood sample, is infected with P. falciparum.

Additionally, the present invention also provides the use of
(i) the protein, preferably the antibody, more preferably the recombinant antibody, according to the present invention;
(ii) the nucleic acid molecule encoding the protein, preferably the antibody, more preferably the recombinant antibody, according to the present invention;
(iii) the vector encoding the nucleic acid molecule according to the present invention;
(iv) the cell expressing the protein, preferably the antibody, more preferably the recombinant antibody, according to the present invention or comprising the vector according to the present invention; or
(v) the pharmaceutical composition according to the present invention as described herein

in (a) the manufacture of a medicament for the treatment or attenuation of malaria, preferably of P. falciparum-malaria or (b) diagnosis of malaria, preferably of P. falciparum-malaria.

The present invention also provides a method for treating a subject, comprising the step of administering to the subject
(i) the protein, preferably the antibody, more preferably the recombinant antibody, according to the present invention;
(ii) the nucleic acid molecule encoding the protein, preferably the antibody, more preferably the recombinant antibody, according to the present invention;
(iii) the vector encoding the nucleic acid molecule according to the present invention;
(iv) the cell expressing the protein, preferably the antibody, more preferably the recombinant antibody, according to the present invention or comprising the vector according to the present invention; or
(v) the pharmaceutical composition according to the present invention as described herein.

In some embodiments the subject may be a human. One way of checking efficacy of therapeutic treatment involves monitoring disease symptoms after administration of the composition of the invention. Treatment can be a single dose schedule or a multiple dose schedule.

Preferably,
(i) the protein, preferably the antibody, more preferably the recombinant antibody, according to the present invention;
(ii) the nucleic acid molecule encoding the protein, preferably the antibody, more preferably the recombinant antibody, according to the present invention;
(iii) the vector encoding the nucleic acid molecule according to the present invention;
(iv) the cell expressing the protein, preferably the antibody, more preferably the recombinant antibody, according to the present invention or comprising the vector according to the present invention; or
(v) the pharmaceutical composition according to the present invention as described herein

is administered to a subject in need of such treatment. Such a subject includes, but is not limited to, one who is particularly at risk of or susceptible to malaria, preferably of P. falciparum-malaria.

Antibodies and fragments thereof as described in the present invention may also be used in a kit for the diagnosis of malaria, preferably of P. falciparum-malaria.

The present invention also provides a method of limiting infection with Plasmodium falciparum, or lowering the risk of Plasmodium falciparum infection, comprising: administering to a subject in need thereof, a therapeutically effective amount of the protein according to the present invention, preferably the antibody according to the present invention as described herein, the nucleic acid according to the present invention, the vector according to the present invention, the cell according to the present invention, or the pharmaceutical composition according to the present invention, preferably the protein according to the present invention, more preferably the antibody according to the present invention as described herein.

The present invention also provides a method of preventing and/or treating malaria in a subject, wherein the method comprises administering to a subject in need thereof the protein according to the present invention, preferably the antibody according to the present invention as described herein, the nucleic acid according to the present invention, the vector according to the present invention, the cell according to the present invention, or the pharmaceutical composition according to the present invention, preferably the protein according to the present invention, more preferably the antibody according to the present invention as described herein.

The present invention is not to be limited in scope by the specific embodiments described herein. Indeed, various modifications of the invention in addition to those described herein will become apparent to those skilled in the art from the description and accompanying figures. Such modifications fall within the scope of the appended claims.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described herein. All publications, patent applications, patents, and other references mentioned herein are incorporated by reference in their entirety. In the case of conflict, the present specification, including definitions, will control.

The following Figures, Sequences and Examples are intended to illustrate the invention further. They are not intended to limit the subject matter of the invention thereto.

Protein Comprising a Mutated LAIR-1 Fragment Binding to an Antigen for use in Prevention and/or Treatment of Various Diseases

In a further aspect, the present invention also provides a protein comprising at least amino acids 67 to 107 of native human LAIR-1, wherein said LAIR-1 fragment comprises at least one mutation in comparison to native human LAIR-1 (SEQ ID NO: 9), said at least one mutation enabling binding to an antigen, and wherein said LAIR-1 fragment shows at least 70% amino acid sequence identity to amino acids 67 to 107 of native human LAIR-1 (SEQ ID NO: 9). Preferably, said LAIR-1 fragment shows at least 75%, more preferably at least 80%, even more preferably at least 85%, most preferably at least 90% amino acid sequence identity to amino acids 67 to 107 of native human LAIR-1 (SEQ ID NO: 9).

Based on the surprising finding that the mutated LAIR-1 fragment as described above enables binding to Plasmodium surface antigens, other/further mutations in the LAIR-1 fragment in particular enable binding to other/further antigens and are, thus, useful in the prevention and/or treatment of various diseases as described herein.

Preferably, such a protein comprises at least amino acids 50 to 110 of native human LAIR-1, wherein said LAIR-1 fragment comprises at least one mutation in comparison to native human LAIR-1 (SEQ ID NO: 11), said at least one mutation enabling binding to an antigen, and wherein said LAIR-1 fragment shows at least 70% amino acid sequence identity to amino acids 50 to 110 of native human LAIR-1 (SEQ ID NO: 11). Preferably, said LAIR-1 fragment shows at least 75%, more preferably at least 80%, even more preferably at least 85%, most preferably at least 90% amino acid sequence identity to amino acids 50 to 110 of native human LAIR-1 (SEQ ID NO: 11).

More preferably, such a protein comprises at least amino acids 40 to 115 of native human LAIR-1, wherein said LAIR-1 fragment comprises at least one mutation in comparison to native human LAIR-1 (SEQ ID NO: 12), said at least one mutation enabling binding to an antigen, and wherein said LAIR-1 fragment shows at least 70% amino acid sequence identity to amino acids 40 to 115 of native human LAIR-1 (SEQ ID NO: 12). Preferably, said LAIR-1 fragment shows at least 75%, more preferably at least 80%, even more preferably at least 85%, most preferably at least 90% amino acid sequence identity to amino acids 40 to 115 of native human LAIR-1 (SEQ ID NO: 12).

Even more preferably, such a protein comprises at least amino acids 30 to 120 of native human LAIR-1, wherein said LAIR-1 fragment comprises at least one mutation in comparison to native human LAIR-1 (SEQ ID NO: 13), said at least one mutation enabling binding to an antigen, and wherein said LAIR-1 fragment shows at least 70% amino acid sequence identity to amino acids 30 to 120 of native human LAIR-1 (SEQ ID NO: 13). Preferably, said LAIR-1 fragment shows at least 75%, more preferably at least 80%, even more preferably at least 85%, most preferably at least 90% amino acid sequence identity to amino acids 30 to 120 of native human LAIR-1 (SEQ ID NO: 13).

Most preferably, such a protein comprises at least amino acids 24 to 121 of native human LAIR-1, wherein said LAIR-1 fragment comprises at least one mutation in comparison to native human LAIR-1 (SEQ ID NO: 14), said at least one mutation enabling binding to an antigen, and wherein said LAIR-1 fragment shows at least 70% amino acid sequence identity to amino acids 24 to 121 of native human LAIR-1 (SEQ ID NO: 14). Preferably, said LAIR-1 fragment shows at least 75%, more preferably at least 80%, even more preferably at least 85%, most preferably at least 90% amino acid sequence identity to amino acids 24 to 121 of native human LAIR-1 (SEQ ID NO: 14).

As used herein, the term “antigen” refers to any structural substance or compound, which serves as a target for the receptors of an adaptive immune response, in particular as a target for antibodies, T cell receptors, and/or B cell receptors. In other words, an “antigen” is typically able to specifically bind to a (naturally occurring) antibody. In particular, an antigen typically causes a (human) immune system to produce antibodies against it. However, some antigens do not, by themselves, elicit antibody production. Preferably, the antigen is selected from the group consisting of: a peptide, a polypeptide, or a protein; a polysaccharide; a lipid; a lipoprotein or a lipopeptide; a glycolipid; a nucleic acid; a small molecule drug; and a toxin.

Preferably, the protein is a recombinant protein. The term “recombinant protein”, as used herein, refers to any protein which is prepared, expressed, created or isolated by recombinant means, and which is not naturally occurring. Preferably, the protein is a fusion protein.

It is also preferred that the protein is an antibody, preferably a recombinant antibody. Thereby, it is particularly preferred that the protein further comprises an Fc moiety as described herein.

Such a protein comprising a mutated LAIR-1 fragment can be used in the prevention and/or treatment of various diseases, in particular in prevention and/or treatment of a disorder and/or a disease selected from the group consisting of infectious diseases, autoimmune diseases, inflammatory diseases and cancers. For example, if collagen-binding of the mutated LAIR-1 fragment is not abolished by the mutations, a protein comprising such a mutated LAIR-1 fragment may be used in prevention and/or treatment of rheumatic diseases, such as ankylosing spondylitis, bursitis, tendinitis, capsulitis, osteoarthritis, rheumatoid arthritis, polychondritis, systemic lupus erythematosus, juvenile arthritis, Sjögren syndrome, scleroderma, polymyositis, dermatomyositis, Behcet's disease, reactive arthritis and psoriatic arthritis, for example due to the binding of the LAIR-1 fragment to collagen. Thereby, such proteins comprising mutated LAIR-1 fragments according to the present invention, in which the collagen-binding of LAIR-1 is not abolished (by the mutation), are preferably used only in such (autoimmune) diseases and/or disorders, in which anti-collagen antibodies do not deteriorate the disease/disorder.

Treatment and/or prevention of infectious diseases is preferred. In particular, such a protein comprising a mutated LAIR-1 fragment may be used for (the preparation of a medicament for) the prophylaxis, treatment and/or amelioration of infectious diseases, preferably viral, retroviral, bacterial or protozoological infectious diseases. Such infectious diseases are typically selected from AIDS, anthrax, Japanese encephalitis, bacterial infectious diseases such as miscarriage (prostate inflammation), anthrax, appendicitis, borreliosis, botulism, Camphylobacter, Chlamydia trachomatis (inflammation of the urethra, conjunctivitis), cholera, diphtheria, donavanosis, epiglottitis, typhus fever, gas gangrene, gonorrhoea, rabbit fever, Heliobacter pylori, whooping cough, climatic bubo, osteomyelitis, Legionnaire's disease, chicken-pox, condyloma acuminata, cytomegalic virus (CMV), dengue fever, early summer meningoencephalitis (ESME), Ebola virus, colds, fifth disease, foot-and-mouth disease, herpes simplex type I, herpes simplex type II, herpes zoster, HSV, infectious diseases caused by parasites, protozoa or fungi, such as amoebiasis, bilharziosis, Chagas disease, Echinococcus, fish tapeworm, fish poisoning (Ciguatera), fox tapeworm, athlete's foot, canine tapeworm, candidosis, yeast fungus spots, scabies, cutaneous Leishmaniosis, lambliasis (giardiasis), lice, malaria, microscopy, onchocercosis (river blindness), fungal diseases, bovine tapeworm, schistosomiasis, porcine tapeworm, toxoplasmosis, trichomoniasis, trypanosomiasis (sleeping sickness), visceral Leishmaniosis, nappy/diaper dermatitis or miniature tapeworm, infectious erythema, influenza, Kaposi's sarcoma, Lassa fever, Leishmaniasis, leprosy, listeriosis, Lyme borreliosis, malaria, Marburg virus infection, measles, meningitis, including bacterial meningitis, molluscum contagiosum, mononucleosis, mumps, Mycoplasma hominis, neonatal sepsis (Chorioamnionitis), noma, Norwalk virus infection, otitis media, paratyphus, Pfeiffer's glandular fever, plague, pneumonia, polio (poliomyelitis, childhood lameness), pseudo-croup, rabies, Reiter's syndrome, Rocky Mountain spotted fever, Salmonella paratyphus, Salmonella typhus, SARS, scarlet fever, shingles, hepatitis, smallpox, soft chancre, syphilis, tetanus,three-day fever, tripper, tsutsugamushi disease, tuberculosis, typhus, vaginitis (colpitis), viral diseases caused by cytomegalovirus (CMV), orthopox variola virus, orthopox alastrim virus, parapox ovis virus, molluscum contagiosum virus, herpes simplex virus 1, herpes simplex virus 2, herpes B virus, varicella zoster virus, pseudorabies virus, human cytomegaly virus, human herpes virus 6, human herpes virus 7, Epstein-Barr virus, human herpes virus 8, hepatitis B virus, chikungunya virus, O'nyong'nyong virus, rubivirus, hepatitis C virus, GB virus C, West Nile virus, dengue virus, yellow fever virus, louping ill virus, St. Louis encephalitis virus, Japan B encephalitis virus, Powassan virus, FSME virus, SARS, SARS-associated corona virus, human corona virus 229E, human corona virus Oc43, Torovirus, human T cell lymphotropic virus type I, human T cell lymphotropic virus type II, HIV (AIDS), i.e. human immunodeficiency virus type 1 or human immunodeficiency virus type 2, influenza virus, Lassa virus, lymphocytic choriomeningitis virus, Tacaribe virus, Junin virus, Machupo virus, Borna disease virus, Bunyamwera virus, California encephalitis virus, Rift Valley fever virus, sand fly fever virus, Toscana virus, Crimean-Congo haemorrhagic fever virus, Hazara virus, Khasan virus, Hantaan virus, Seoul virus, Prospect Hill virus, Puumala virus, Dobrava Belgrade virus, Tula virus, sin nombre virus, Lake Victoria Marburg virus, Zaire Ebola virus, Sudan Ebola virus, Ivory Coast Ebola virus, influenza virus A, influenza virus B, influenza viruses C, parainfluenza virus, measles virus, mumps virus, respiratory syncytial virus, human metapneumovirus, vesicular stomatitis Indiana virus, rabies virus, Mokola virus, Duvenhage virus, European bat lyssavirus 1+2, Australian bat lyssavirus, adenoviruses A-F, human papilloma viruses, condyloma virus 6, condyloma virus 11, polyoma viruses, adeno-associated virus 2, rotaviruses, or orbiviruses, Varicella including Varizella zoster, and malaria virus, viral infectious diseases such as AIDS, infectious diseases caused by Condyloma acuminata, hollow warts, Dengue fever, three-day fever, Ebola virus, cold, early summer meningoencephalitis (FSME), flu, shingles, hepatitis, herpes simplex type I, herpes simplex type II, Herpes zoster, influenza, Japanese encephalitis, Lassa fever, Marburg virus, warts, West Nile fever, yellow fever, etc.

Examples of infectious diseases include diseases caused by viruses, bacteria, fungi, protozoa and multicellular parasites. They include, for instance, Amoebiasis, Anthrax, Buruli Ulcer (Mycobacterium ulcerans), Caliciviruses associated diarrhoea, Campylobacter diarrhoea, Cervical Cancer (Human papillomavirus), Chlamydia trachomatis associated genital diseases, Cholera , Crimean-Congo haemorrhagic fever, Dengue Fever, Diptheria, Ebola haemorrhagic fever, Enterotoxigenic Escherichia coli (ETEC) diarrhoea, Gastric Cancer (Helicobacter pylori), Gonorrhea, Group A Streptococcus associated diseases, Group B Streptococcus associated diseases, Haemophilus influenzae B pneumonia and invasive disease, Hepatitis A, Hepatitis B, Hepatitis C, Hepatitis E diarrhoea, Herpes simplex type 2 genital ulcers, HIV/AIDS, Hookworm Disease, Influenza, Japanese encephalitis, Lassa Fever, Leishmaniasis, Leptospirosi, Liver cancer (Hepatitis B), Liver Cancer (Hepatitis C), Lyme Disease, Malaria, Marburg haemorrhagic fever, Measles, Mumps, Nasopharyngeal cancer (Epstein-Barr virus), Neisseria meningitidis Meningitis, Parainfluenza associated pneumonia, Pertussis, Plague, Poliomyelitis, Rabies, Respiratory syncytial virus (RSV) pneumonia, Rift Valley fever, Rotavirus diarrhoea, Rubella, Schistosomiasis, Severe Acute Respiratory Syndrome (SAKS), Shigellosis, Smallpox, Staphylococcus aureus associated diseases, Stomach Cancer (Helicobacter pylori), Streptococcus pneumoniae and invasive disease, Tetanus, Tick-borne encephalitis, Trachoma, Tuberculosis, Tularaemia, Typhoid fever, West-Nile virus associated disease, Yellow fever.

In particular, such a protein comprising a mutated LAIR-1 fragment may be used for (the preparation of a medicament for) the prophylaxis, treatment and/or amelioration of autoimmune disorders, for example autoimmune diseases of the CNS, auto-inflammatory diseases, Celiac disease; Sjogren's syndrome, systemic lupus erythematosus etc. Typically, autoimmune diseases arise from an abnormal immune response of the body against substances and tissues normally present in the body (autoimmunity). This may be restricted to certain organs (e.g. in autoimmune thyroiditis) or may involve a particular tissue in different places (e.g. Goodpasture's disease which may affect the basement membrane in both the lung and the kidney). Autoimmune diseases may be classified by corresponding type of hypersensitivity: type I (i.e. urticaria induced by autologous serum), type II, type III, or type IV. Preferably, such proteins comprising mutated LAIR-1 fragments according to the present invention, in which the collagen-binding of LAIR-1 is not abolished (by the mutation), are used only in such (autoimmune) diseases and/or disorders, in which anti-collagen antibodies do not deteriorate the disease/disorder.

Examples of autoimmune diseases include Blau syndrome, Bullous pemphigoid, Cancer, Castleman's disease, Celiac disease, Chagas disease, Chronic inflammatory demyelinating polyneuropathy, Chronic recurrent multifocal osteomyelitis, chronic obstructive pulmonary disease, Churg-Strauss syndrome, Cicatricial pemphigoid, Cogan syndrome, Cold agglutinin disease, Complement component 2 deficiency, Contact dermatitis, Cranial arteritis, CREST syndrome, Crohn's disease, Cushing's Syndrome, Dercum's disease, Dermatitis herpetiformis, Dermatomyositis, Diabetes mellitus type 1, Diffuse cutaneous systemic sclerosis, Dressler's syndrome, lupus, Discoid lupus erythematosus, Eczema, Acute disseminated encephalomyelitis (ADEM), Addison's disease, Agammaglobulinemia, Amyotrophic lateral sclerosis (Also Lou Gehrig's disease; Motor Neuron Disease), Ankylosing Spondylitis Antiphospholipid syndrome, Antisynthetase syndrome, Atopic dermatitis, Autoimmune aplastic anemia, Autoimmune cardiomyopathy, Autoimmune hemolytic anemia, Autoimmune hepatitis, Autoimmune inner ear disease, Autoimmune lymphoproliferative syndrome, Autoimmune peripheral neuropathy, Autoimmune pancreatitis, Autoimmune polyendocrine syndrome, Autoimmune progesterone dermatitis, Autoimmune thrombocytopenic purpura, Autoimmune urticarial, Autoimmune uveitis, Balo disease/Balo concentric sclerosis, Behcet's disease, Berger's disease, Bickerstaff's encephalitis, Endometriosis, Enthesitis-related arthritis, Eosinophilic gastroenteritis, Epidermolysis bullosa acquisita, Erythroblastosis fetalis, Evan's syndrome, Fibrodysplasia ossificans, Fibrosing alveolitis (or Idiopathic pulmonary fibrosis), Gastritis, Glomerulonephritis, Goodpasture's syndrome, Graves' disease, Guillain-Barre syndrome, Hashimoto's encephelopathy, Hashimoto's thyroiditis, Gestational Pemphigoid, Hidradenitis suppurativa, Hypogammaglobulinemia, Idiopathic thrombocytopenic purpura (Autoimmune thrombocytopenic purpura), IgA nephropathy, Occular cicatricial pemphigoid, Inclusion body myositis, Rheumatoid arthritis, Chronic inflammatory Rheumatic fever, demyelinating polyneuropathy, Sarcoidosis, Palindromic rheumatism, Interstitial cystitis, Juvenile idiopathic Schizophrenia, PANDAS (pediatric arthritis aka Juvenile autoimmune rheumatoid arthritis), Schmidt syndrome, neuropsychiatric Kawasaki's disease another form of APS, Schnitzler syndrome, Paraneoplastic cerebellar myasthenic syndrome, Leukocytoclastic Serum Sickness, Lichen planus, Sjogren's syndrome, Lichen sclerosus, Parsonage-Tumer, Linear IgA disease, Still's disease, Pemphigus vulgaris, Lupoid hepatitis, Autoimmune hepatitis, Stiff person syndrome, Pernicious anaemia, Subacute bacterial endocarditis (SBE), POEMS syndrome, Lupus erythematosus, Sweet's syndrome, Sympathetic ophthalmia, Meniere's disease, Systemic lupus, Primary biliary cirrhosis, Miller-Fisher syndrome, Takayasu's arteritis, cholangitis, Progressive inflammatory neuropathy, Mucha-Habermann disease, Psoriasis, Psoriatic arthritis, Pyoderma gangrenosum, Multiple sclerosis, Pure red cell aplasia, Rasmussen's encephalitis, Myasthenia gravis, Transverse myelitis, Raynaud phenomenon, Microscopic colitis, Ulcerative colitis, Myositis, idiopathic inflammatory bowel disease (IBD), Neuromyelitis optica, Devic's disease, and Neuromyotonia.

In particular, such a protein comprising a mutated LAIR-1 fragment may be used for (the preparation of a medicament for) the prophylaxis, treatment and/or amelioration of cancer or tumor diseases, including diseases caused by defective apoptosis, preferably selected from acusticus neurinoma, anal carcinoma, astrocytoma, basalioma, Behcet's syndrome, bladder cancer, blastomas, bone cancer, brain metastases, brain tumors, brain cancer (glioblastomas), breast cancer (mamma carcinoma), Burkitt's lymphoma, carcinoids, cervical cancer, colon carcinoma, colorectal cancer, corpus carcinoma, craniopharyngeomas, CUP syndrome, endometrial carcinoma, gall bladder cancer, genital tumors, including cancers of the genitourinary tract, glioblastoma, gliomas, head/neck tumors, hepatomas, histocytic lymphoma, Hodgkin's syndromes or lymphomas and non-Hodgkin's lymphomas, hypophysis tumor, intestinal cancer, including tumors of the small intestine, and gastrointestinal tumors, Kaposi's sarcoma, kidney cancer, kidney carcinomas, laryngeal cancer or larynx cancer, leukemia, including acute myeloid leukaemia (AML), erythroleukemia, acute lymphoid leukaemia (ALL), chronic myeloid leukaemia (CML), and chronic lymphocytic leukaemia (CLL), lid tumor, liver cancer, liver metastases, lung carcinomas (=lung cancer=bronchial carcinoma), small cell lung carcinomas and non-small cell lung carcinomas, and lung adenocarcinoma, lymphomas, lymphatic cancer, malignant melanomas, mammary carcinomas (=breast cancer), medulloblastomas, melanomas, meningiomas, Mycosis fungoides, neoplastic diseases neurinoma, oesophageal cancer, oesophageal carcinoma (=oesophageal cancer), oligodendroglioma, ovarian cancer (=ovarian carcinoma), ovarian carcinoma, pancreatic carcinoma (=pancreatic cancer), penile cancer, penis cancer, pharyngeal cancer, pituitary tumour, plasmocytoma, prostate cancer (=prostate tumors), rectal carcinoma, rectal tumors, renal cancer, renal carcinomas, retinoblastoma, sarcomas, Schneeberger's disease, skin cancer, e.g. melanoma or non-melanoma skin cancer, including basal cell and squamous cell carcinomas as well as psoriasis, pemphigus vulgaris, soft tissue tumours, spinalioma, stomach cancer, testicular cancer, throat cancer, thymoma, thyroid carcinoma, tongue cancer, urethral cancer, uterine cancer, vaginal cancer, various virus-induced tumors such as, for example, papilloma virus-induced carcinomas (e.g. cervical carcinoma=cervical cancer), adenocarcinomas, herpes virus-induced tumors (e.g. Burkitt's lymphoma, EBV-induced B-cell lymphoma, cervix carcinoma), heptatitis B-induced tumors (hepatocell carcinomas), HTLV-1- and HTLV-2-induced lymphomas, vulval cancer, wart conditions or involvement, etc. In the present context, the terms “therapy” and “therapeutic” preferably mean to have at least some minimal physiological effect upon being administered to a living body. For example, a physiological effect upon administering a “therapeutic” anti-tumor compound may be the inhibition of tumor growth, or decrease in tumor size, or prevention reoccurrence of the tumor. Preferably, in the treatment of cancer or neoplastic disease, a compound which inhibits the growth of a tumor or decreased the size of the tumor or prevents the reoccurrence of the tumor would be considered therapeutically effective. The term “anti-tumor drug” therefore preferably means any therapeutic agent having therapeutic effect against a tumor, neoplastic disease or cancer.

Examples of cancers include brain cancer, prostate cancer, breast cancer, ovarian cancer, esophageal cancer, lung cancer, liver cancer, kidney cancer, melanoma, gut carcinoma, lung carcinoma, head and neck squamous cell carcinoma, chronic myeloid leukemia, colorectal carcinoma, gastric carcinoma, endometrial carcinoma, myeloid leukemia, lung squamous cell carcinoma, acute lymphoblastic leukemia, acute myelogenous leukemia, bladder tumor, promyelocytic leukemia, non-small cell lung carcinoma, sarcoma.

The cancer may be a solid tumor, blood cancer, or lymphatic cancer. The cancer may be benign or metastatic.

In particular, such a protein comprising a mutated LAIR-1 fragment may be used for (the preparation of a medicament for) the prophylaxis, treatment and/or amelioration of inflammatory diseases.

Examples of inflammatory diseases include Alzheimer's disease, ankylosing spondylitis, arthritis (osteoarthritis, rheumatoid arthritis (RA), psoriatic arthritis), rheumatic diseases, asthma, atherosclerosis, Crohn's disease, colitis, dermatitis, diverticulitis, fibromyalgia, hepatitis, irritable bowel syndrome (IBS), systemic lupus erythematous (SLE), nephritis, Parkinson's disease, ulcerative colitis.

Accordingly, the present invention also provides a method of preventing and/or treating a disorder and/or a disease selected from the group consisting of infectious diseases, autoimmune diseases, inflammatory diseases and cancers in a subject, wherein the method comprises administering to a subject in need thereof the protein as described herein.

### BRIEF DESCRIPTION OF THE FIGURES

In the following a brief description of the appended figures will be given. The figures are intended to illustrate the present invention in more detail. However, they are not intended to limit the subject matter of the invention in any way.

FIG. 1 shows for Example 1 an example of staining of P. falciparum-infected erythrocytes by a broadly cross-reactive antibody (MGD21). IEs are stained with SYBR Green I dye (DNA) to discriminate them from uninfected erythrocytes used as control. The graph shows that MGD21 specifically binds only to IEs.

FIG. 2 shows an alignment of selected monoclonal antibodies of Example 1 (antibodies MGD21, MGD39, MGD47 and MGD55 in FIG. 2A and antibodies MGC1, MGC7, MGC37 and MGC29 in FIG. 2B) to an amino acid sequence encoded by the corresponding fragment of genomic LAIR1 sequence (exon+intron).

FIG. 3 shows a scheme of the different antibody variants constructed in Example 3. The different elements of the 10 antibody constructs are compared to MGD21 and FI499 (unrelated antibody). MGD21 binds to erythrocytes infected with 9/9 primary P. falciparum isolates and carries the LAIR-1 exon+intron insertion. F1499 is an IgG antibody that binds influenza hemagglutinin and uses different V, D and J elements. Dα and Dβ indicate two putative D elements. GGGGS is an artificial linker. VH4-4, JH6, VK1-8 and JK5 and LAIR-1 intron and exon were also tested in the germline form (GL). For LAIR-1 the genomic sequence (ENSG00000167613) was used. In the right column, it is indicated whether the antibody (construct) binds to IEs as tested in Example 4.

FIG. 4 shows the results of Example 4 indicating that the mutated LAIR-1 exon is the only element required for mAb MGD21 binding to P. falciparum-infected erythrocytes. The antibodies were quantitated and tested for their capacity to stain IEs. “Con1” refers to “FI499_DexinDJ”, “Con2” refers to “FI499VJ_DexinD”, “Con3” refers to “MGD21_exin_longGS”, “Con4” refers to “MGD21_exin_shortGS”, “Con5” refers to “MGD21_NOexin”, “Con6” refers to “MGD21_NOin”, “Con7” refers to “MGD21_NOVD”, “Con8” refers to “MGD21 GL_exinWT”, “Con9” refers to “MGD21_wholeGL”.

FIG. 5 shows a scheme of the different fusion proteins produced in Example 5. M1, M2, M3 and M4 are four different mouse IgG2b fusion proteins comprising the mutated LAIR-1 fragment according to the present invention, while H1 and H2 are two different human IgG1 fusion proteins comprising the mutated LAIR-1 fragment according to the present invention. M1 and H1 share the same variable region. M4 and H2 share the same variable region. “D_{α}” and “D_{β}” refer to the expression products of a first fragment and of a second fragment, different from the first fragment, of the same or different D (Diversity) gene segment element of a heavy chain variable region of an IgG-type antibody. “JH6” refers to the expression product of a J (Joining) gene segment element of a heavy chain variable region of an IgG-type antibody. “Exon” refers to the mutated LAIR-1 fragment. “Intron” and “Intron_{α}” refer to further LAIR-1 elements (expression products from one LAIR-1 intron fragment, whereby “Intron_{α}” is a fragment of “Intron”). “Hinge”, “CH2”, and “CH3” form together the constant region provided by the plasmid.

FIG. 6 shows for Example 6 that the mutated LAIR-1 fragment expressed as a fusion protein (cf. Example 5) binds to IEs. The four fusion proteins expressed in the mouse IgG2b fusion-protein vector were quantitated and tested for their capacity to stain IEs.

FIG. 7 shows for Example 7 that fusion proteins comprising the mutated LAIR-1 fragment efficiently opsonize P. falciparum-infected erythrocytes. Parasites were stained with DAPI and mixed with a titration of antibodies and fusion proteins, followed by incubation with monocytes at 37° C. for 1 hour. Monocytes were stained with anti-CD14-APC and MFI of DAPI (A) and the % of DAPI-positive monocytes (B) were calculated in CD14-positive populations. “DexinDJ” and “exon” are two fusion proteins expressed in the human IgG1 vector (cf. Example 5, also referred to as H1 and H2). F1499 is an unrelated antibody used as control. FIG. 7C shows agglutinates of 3D7-MGD21⁺ or 11019-MGD21⁺ IEs formed by MGD21 or MGC34. Scale bar, 25 μm.

FIG. 8 shows for Example 7 that antibodies MGD21, MG47, MGD55, MGC28 and MGC34 efficiently opsonize P. falciparum-infected erythrocytes. The IEs were stained with 4′,6-diamidino-2-phenylindole (DAPI), which was quantified in monocytes as a measure of phagocytosis. (A) Opsonic phagocytosis of 3D7-MGD2⁺ IEs by monocytes (n=3 for MGD21, MGD21 LALA and BKC3, n=2 for others). (B) Opsonic phagocytosis of 11019-MGD21⁺ IEs by monocytes (n=2).

FIG. 9 shows for Example 8 an alignment of the mutated LAIR-1 exon of the human monoclonal antibodies of Example 1 with amino acids 24 to 121 of native human LAIR-1 (SEQ ID NO: 14). Positions T67, N69, A77, P106 and P107 are shown in frames.

FIG. 10 shows for Example 8 the mutated LAIR-1 fragment modeled on the structure of the LAIR-1 extracellular domain. The LAIR-1 structure is shown as cartoon (left) and as surface (right). The five positions, at which a mutation may occur in the mutated LAIR-1 fragment as compared to the native LAIR-1 structure are highlighted in black.

FIG. 11 shows for Example 9 that the LAIR-1 fragment expressed as a fusion protein and carrying different combinations of mutations at positions T67, N69, A77, P106 and P107 binds to IEs while the same LAIR-1 fragment with no mutations does not bind to IEs. “LAIR1 ex” is the fusion protein carrying the LAIR1 fragment corresponding to the genomic sequence (Gene: LAIR1 ENSG00000167613). “LAIR1ex+X” are the fusion protein carrying the LAIR-1 fragment corresponding to the genomic sequence with one or more mutations (only mutated residues [L,S1,T,S2,R] are indicated according to the 5 most preferred mutations respectively: T67L, N695, A77T, P106S, and P107R, whereby “L” refers to T67L, “S1” refers to N69S, “T” refers to A77T, “S2” refers to P106S and “R” refers to P107R. For instance “LAIR1ex+L” carries the mutation T67L and “LAIR1ex+S2” carries the mutation P106S.

FIG. 12 shows for Example 10 that the different mutations in the LAIR-1 fragment expressed as a fusion protein and carrying different combinations of mutations at positions T67, N69, A77, P106 and P107 (cf. Example 9) influence binding to collagen. The fusion proteins are the same shown in FIG. 10 (cf. Example 9).

FIG. 13 depicts a western blot showing MGD21 binding to erythrocyte ghosts and MGD21 immunoprecipitates (IP) prepared from 3D7-MGD21⁻ and 3D7-MGD21⁻ IEs (representative of n=2 independent experiments). Controls include uninfected erythrocytes (uEs) and immunoprecipitates with an irrelevant antibody (BKC3). Specific bands are marked with asterisks. Anti-human IgG was used as the secondary antibody, resulting in detection of antibodies used for immunoprecipitation alongside antigens of interest. Numbers on right indicate kDa.

FIG. 14 shows a Volcano plot from LC-MS analysis of MGD21 immunoprecipitates prepared from 3D7-MGD21⁺ IEs versus from 3D7-MGD21⁻ IEs (from n=4 independent experiments). Statistical significance was evaluated by Welch tests (P<0.01 for PF3 D7_1400600).

FIG. 15 shows a heat map from LC-MS analysis showing RIFIN expression levels (calculated as intensity-based absolute quantification (iBAQ) scores) in erythrocyte ghosts prepared from 3D7-MGD21⁺ and 3D7-MGD21⁻ IEs (two experiments shown). Boxes with crosses indicate that expression levels are below the detection limit.

FIG. 16 shows the percentage of IEs (representative of n=2 independent experiments) stained by the antibodies. BKC3 is a negative control antibody.

FIG. 17 shows for Example 11 a western blot (A) showing MGD21 binding to immunoprecipitates (IP) prepared from 9605-MGD21⁻ and 9605-MGD21⁺ IEs (representative of n=2 independent experiments). Specific bands are marked with an asterisk. Anti-human IgG was used as the secondary antibody, resulting in detection of antibodies used for immunoprecipitation alongside antigens of interest. FIG. 16B shows percentage of 9605-MGD21⁻ and 9605-MGD21⁺ IEs recognized by representative MGC and MGD antibodies (representative of n=2 independent experiments).

FIG. 18 shows (A) the percentage of transfected CHO cells (n=1) stained by the antibodies. BKC3 is a negative control antibody. FIG. 17B shows MGD21 and BKC3 staining of CHO cells transfected with a specific (PF3D7_1400600) or an irrelevant (PF3D7_0100200) RIFIN (representative of n=5 independent experiments).

FIG. 19 shows binding of MGD21 (left) or of an Fc fusion protein containing the LAIR1 domain of MGD21 (right) to CHO cells transfected with RIFINs (PF3D7_1400600 and PF3D7_0100200), a RIFIN chimaera containing the constant region of PF3D7_0100200 and the variable region of PF3D7_1400600 (PF3D7_0100200c_1400600v), or the inverse chimaera (PF3D7_1400600c_0100200v) (n=1).

### EXAMPLES

In the following, particular examples illustrating various embodiments and aspects of the invention are presented. However, the present invention shall not to be limited in scope by the specific embodiments described herein. The following preparations and examples are given to enable those skilled in the art to more clearly understand and to practice the present invention. The present invention, however, is not limited in scope by the exemplified embodiments, which are intended as illustrations of single aspects of the invention only, and methods which are functionally equivalent are within the scope of the invention. Indeed, various modifications of the invention in addition to those described herein will become readily apparent to those skilled in the art from the foregoing description, accompanying figures and the examples below. All such modifications fall within the scope of the appended claims.

### Example 1

### Isolation of Human Monoclonal Antibodies that Broadly React with P. falciparum-Infected Erythrocytes (IE)

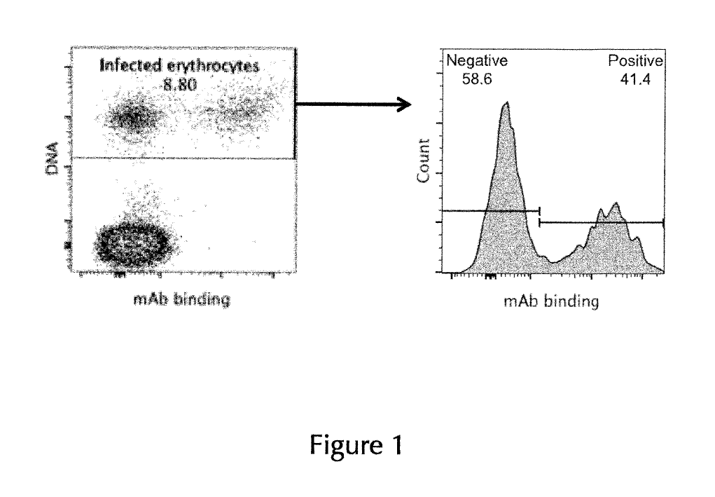

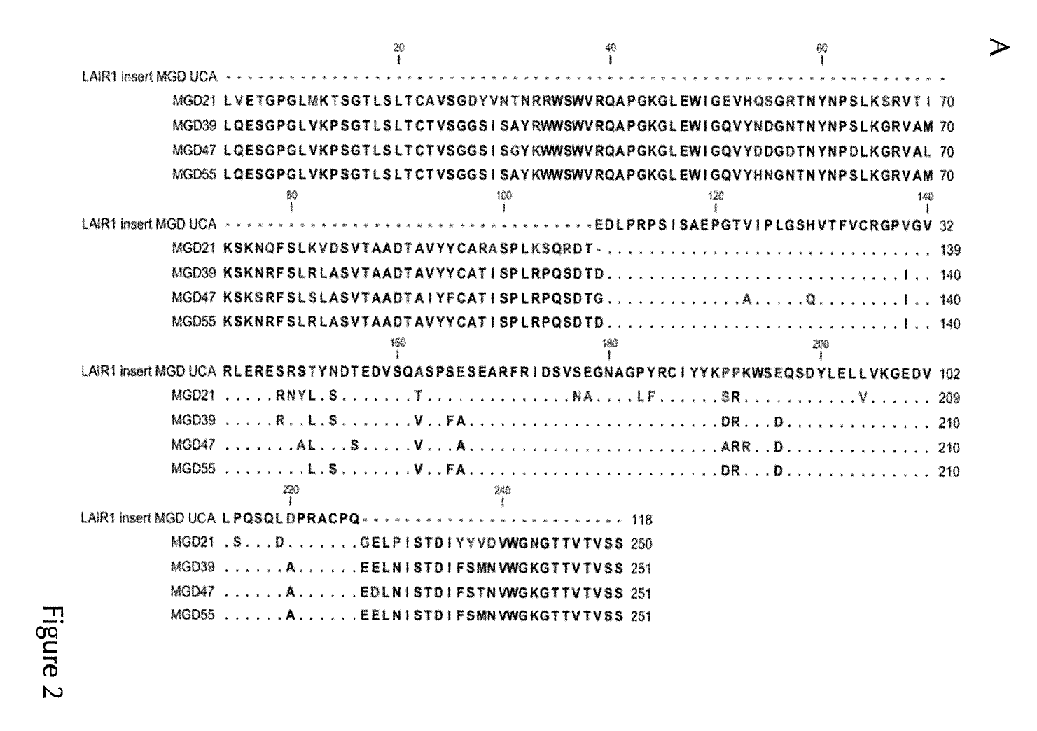

Two African donors (identified as donor C and D) were selected for their high levels of serum antibodies capable of cross-agglutinating erythrocytes infected with different field isolates of P. falciparum. Memory B cells were isolated and immortalized as described by Traggiai, E., et al. An efficient method to make human monoclonal antibodies from memory B cells: potent neutralization of SARS coronavirus. Nat. Med. 10, 871-875 (2004) to isolate monoclonal antibodies. Briefly, memory B cells were isolated from cryopreserved PBMCs using anti-FITC microbeads following staining of PBMCs with CD22-FITC, and were immortalized with Epstein-Barr virus and CpG in multiple wells. After 14 days culture supernatants were screened using a high throughput flow cytometer for their capacity to stain infected erythrocytes (IEs): IEs are stained with SYBR Green I dye (DNA) to discriminate them from uninfected erythrocytes used as control. Supernatants are added on top of IEs and binding of specific antibodies is detected using a secondary-anti-human IgG (Fc-specific) antibody. Positive cultures were expanded and the VH and VL genes from individual clones were sequenced. Several antibodies showed a broad reactivity with the different isolates, while others were specific for a single isolate. The reactivity of the panel of antibodies isolated from donor C and donor D with erythrocytes infected with 8 different field isolates of P. falciparum (9106, 9605, 11019, 9215, 9775, 10975, 10936 and 11014) is shown below in Table 7. An example of IE staining is shown in FIG. 1.

Table 7 shows the panel of antibodies isolated from donor C and donor D (“MGC1”-“MGD56”; Table2) and their reactivity with erythrocytes infected with 8 different field isolates of P. falciparum (9106, 9605, 11019, 9215, 9775, 10975, 10936 and 11014). The numbers indicate the % of IEs that stained positive for the different antibodies. nd=not detectable.

| | | % parasite recognition | | | | | | | |
|---|---|---|---|---|---|---|---|---|---|
| | | 9106 | 9605 | 11019 | 9215 | 9775 | 10975 | 10936 | 11014 |
| Donor | MGC1 | 6.7 | 19.5 | 32.7 | 14.9 | 5.7 | 1.4 | 2.0 | 3.4 |
| C | MGC2 | 5.6 | 22.4 | 11.9 | 28.8 | 2.9 | 2.6 | 2.8 | 1.5 |
| | MGC4 | 6.7 | 22.2 | 31.1 | 21.7 | 6.1 | 6.7 | 2.3 | 3.6 |
| | MGC5 | 6.6 | 20.3 | 37.6 | 26.4 | 6.0 | 3.8 | 2.1 | 2.9 |
| | MGC7 | 6.9 | 22.8 | 13.8 | 19.3 | 4.6 | 0.7 | 1.7 | 2.9 |
| | MGC17 | 1.3 | 6.8 | 7.1 | 16.7 | 2.5 | 1.5 | 2.4 | 1.6 |
| | MGC26 | 8.5 | 21.1 | 50.8 | 9.5 | 3.4 | 7.3 | 3.6 | 5.4 |
| | MGC28 | 7.5 | 20.9 | 30.0 | 10.8 | 9.8 | 12.3 | 3.0 | 2.8 |
| | MGC29 | 6.7 | 21.8 | 48.8 | 26.9 | 8.2 | 10.5 | 3.9 | 3.7 |
| | MGC32 | 7.8 | 22.9 | 38.1 | 13.1 | 7.9 | 3.2 | 2.9 | 4.5 |
| | MGC33 | 7.5 | 22.3 | 23.5 | 11.5 | 9.7 | 11.5 | 3.6 | 2.9 |
| | MGC34 | 6.8 | 23.7 | 34.1 | 27.1 | 17.5 | 15.2 | 11.3 | 11.4 |
| | MGC35 | 6.5 | 15.9 | 3.2 | 19.5 | 7.2 | 7.4 | 2.5 | 3.6 |
| | MGC36 | 6.9 | 17.9 | 17.9 | 12.4 | 6.2 | 8.6 | 2.7 | 4.6 |
| | MGC37 | 7.2 | 22.2 | 51.8 | 9.9 | 4.0 | 7.5 | 4.1 | 5.8 |
| Donor | MGD21 | 3.9 | 24.2 | 41.4 | 47.4 | 11.4 | 6.5 | 6.9 | 9.0 |
| D | MGD23 | 5.7 | 14.7 | 7.8 | 11.4 | 7.3 | 3.4 | 4.3 | 6.3 |
| | MGD30 | 4.2 | 7.4 | 4.4 | 9.0 | 5.6 | 6.5 | 2.6 | 3.4 |
| | MGD33 | 4.3 | 12.3 | 9.6 | 15.5 | 8.5 | 14.2 | 6.1 | 7.0 |
| | MGD34 | 5.0 | 28.4 | 46.6 | 35.7 | 16.0 | 11.2 | 8.1 | 13.0 |
| | MGD35 | 6.1 | 3.6 | 6.3 | nd | nd | nd | nd | nd |
| | MGD39 | 13.7 | 31.7 | 43.0 | 37.4 | 15.0 | 14.1 | 10.5 | 11.5 |
| | MGD41 | 3.8 | 17.2 | 6.8 | 14.7 | 8.6 | 7.3 | 6.1 | 6.6 |
| | MGD47 | 10.7 | 28.7 | 24.6 | 22.3 | 14.4 | 11.2 | 11.3 | 10.2 |
| | MGD55 | 14.3 | 37.2 | 33.1 | 38.8 | 19.4 | 15.6 | 13.3 | 14.7 |
| | MGD56 | 3.3 | 17.3 | 4.3 | 12.0 | 6.6 | 6.7 | 2.4 | 9.5 |
| | | <2% | 2-5% | 5-10% | 10-20% | 20-40% | >40% | | |

### Example 2

### The Human Monoclonal Antibodies that Broadly React with P. falciparum-Infected Erythrocytes are Characterized by a Large HCDR3 Containing a Mutated LAIR-1 Exon

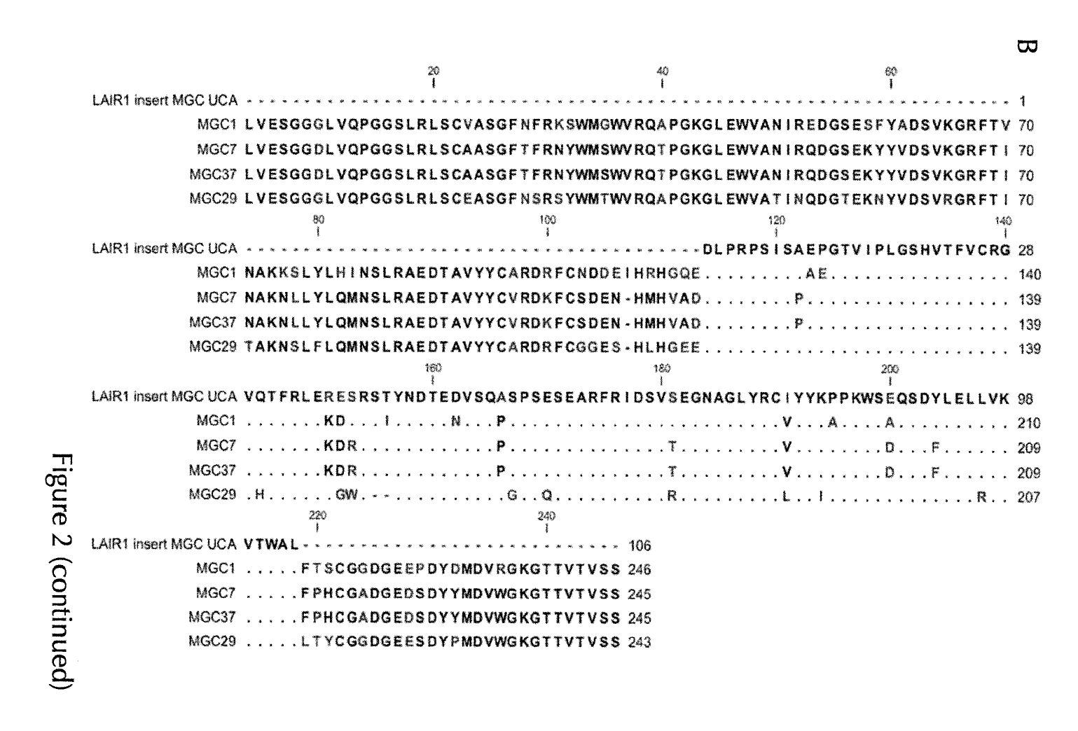

The VH and VL sequences of all of the IE-specific human mAbs of Example 1 were aligned and the V, D and elements identified using the IMGT database. Surprisingly, all the broadly reactive mAbs isolated from both donors were characterized by an extraordinary long CDRH3 ranging from 120 to 130 amino acids, i.e. broadly reactive antibodies had an insert of more than 100 amino acids between the V and DJ segments, whereas narrowly reactive antibodies showed classical VD) organization of the heavy (H) chain gene. The middle and main part of this CDR3 was found to be highly homologous (92% to 98%) to the third exon plus a intronic sequence of LAIR1, a gene encoding an inhibitory receptor specific for collagen which is present on chromosome 19. The aminoacidic alignment of these unusual heavy chain variable regions (VH) is shown with reference to the genomic elements (exon and intron) of the LAIR1 gene (NCBI Reference Sequence: NC_018930.2) in FIG. 2 (cf. FIG. 2: alignment of the complete variable regions of selected antibodies to the genomic LAIR-1 portion corresponding to the inserts. LAIR-1 gene: ENSG00000167613). In addition, the LAIR-1 exon/intron insert was associated with VH4-4 and JH6 in donor D, and with VH3-7 and JH6 in donor C. All the antibodies carried several mutations both in the VD) elements and in the LAIR-1 insert. In both donors, the length and composition of VH and VL and the pattern of mutations define sister clones carrying different levels of mutations (Table 8).

Table 8 below shows the VH and VL gene usage of antibodies.

| | | Heavy chain | | Light chain | |
|---|---|---|---|---|---|
| | | VH | JH | VL | JL |
| Donor | MGC4 | IGHV3-7 | IGHJ6 | IGLV7-43 | IGLJ3 |
| C | MGC5 | IGHV3-7 | IGHJ6 | IGLV7-43 | IGLJ3 |
| | MGC8 | 1GHV3-7 | IGHJ6 | IGLV7-43 | IGLJ3 |
| | MGC29 | IGHV3-7 | IGHJ6 | IGLV7-43 | IGLJ3 |
| | MGC33 | IGHV3-7 | IGHJ6 | IGLV7-43 | IGLJ3 |
| | MGC34 | IGHV3-7 | IGHJ6 | IGLV7-43 | IGLJ3 |
| | MGC35 | IGHV3-7 | IGHJ6 | IGLV7-43 | IGLJ3 |
| | MGC36 | 1GHV3-7 | IGHJ6 | IGLV7-43 | IGLJ3 |
| | MGC2 | IGHV3-7 | IGHJ6 | IGKV1-5 | IGKJ2 |
| | MGC26 | IGHV3-7 | IGHJ6 | IGKV1-5 | IGKJ2 |
| | MGC37 | IGHV3-7 | IGHJ6 | IGKV1-5 | IGKJ2 |
| | MGC1 | IGHV3-7 | IGHJ6 | IGKV4-1 | IGKJ2 |
| | MGC17 | IGHV3-7 | IGHJ6 | IGKV4-1 | IGKJ2 |
| | MGC32 | IGHV3-7 | IGHJ6 | IGKV4-1 | IGKJ2 |
| | MGC7 | IGHV3-7 | IGHJ6 | IGKV1-12 | IGKJ4 |
| Donor | MGD21 | IGHV4-4 | IGHJ6 | IGKV1-8 | IGKJ5 |
| D | MGD23 | IGHV4-4 | IGHJ6 | IGKV1-8 | IGKJ5 |
| | MGD30 | IGHV4-4 | IGHJ6 | IGKV1-8 | IGKJ5 |
| | MGD33 | IGHV4-4 | IGHJ6 | IGKV1-8 | IGKJ5 |
| | MGD34 | IGHV4-4 | IGHJ6 | IGKV1-8 | IGKJ5 |
| | MGD35 | IGHV4-4 | IGHJ6 | IGKV1-8 | IGKJ5 |
| | MGD39 | IGHV4-4 | IGHJ6 | IGKV1-8 | IGKJ5 |
| | MGD41 | IGHV4-4 | IGHJ6 | IGKV1-8 | IGKJ5 |
| | MGD47 | IGHV4-4 | IGHJ6 | IGKV1-8 | IGKJ5 |
| | MGD55 | IGHV4-4 | IGHJ6 | IGKV1-8 | IGKJ5 |
| | MGD56 | IGHV4-4 | IGHJ6 | IGKV1-8 | IGKJ5 |

### Example 3

### Construction of Antibody Variants of MGD21

Of the antibodies described in Example 1 and Example 2 one broadly binding antibody, namely MGD21, was selected. MGD21 (SEQ ID NOs: 390-407) is a monoclonal antibody that binds to erythrocytes infected with 8/8 primary P. falciparum isolates and carries the LAIR-1 exon+intron insertion (a part of the intron, intron_{α}, is shared with MGC antibodies, while the second part, intronp, is shared only with MGD antibodies). To understand which elements are required for binding to IEs, variants of the MGD21 mAb were produced, in which single elements (V, D, J and LAIR-1 exon and intron insertions) were either deleted or substituted with corresponding elements taken from an irrelevant antibody (F1499 reactive to influenza virus hemagglutinin, HA). In addition, variants were produced, in which somatic mutations were reverted to the germline configuration. In particular, mutations in the LAIR-1 exon+intron insertion were reverted to the corresponding original genomic sequence of LAIR-1 gene (NCBI Reference Sequence: NC_018930.2).

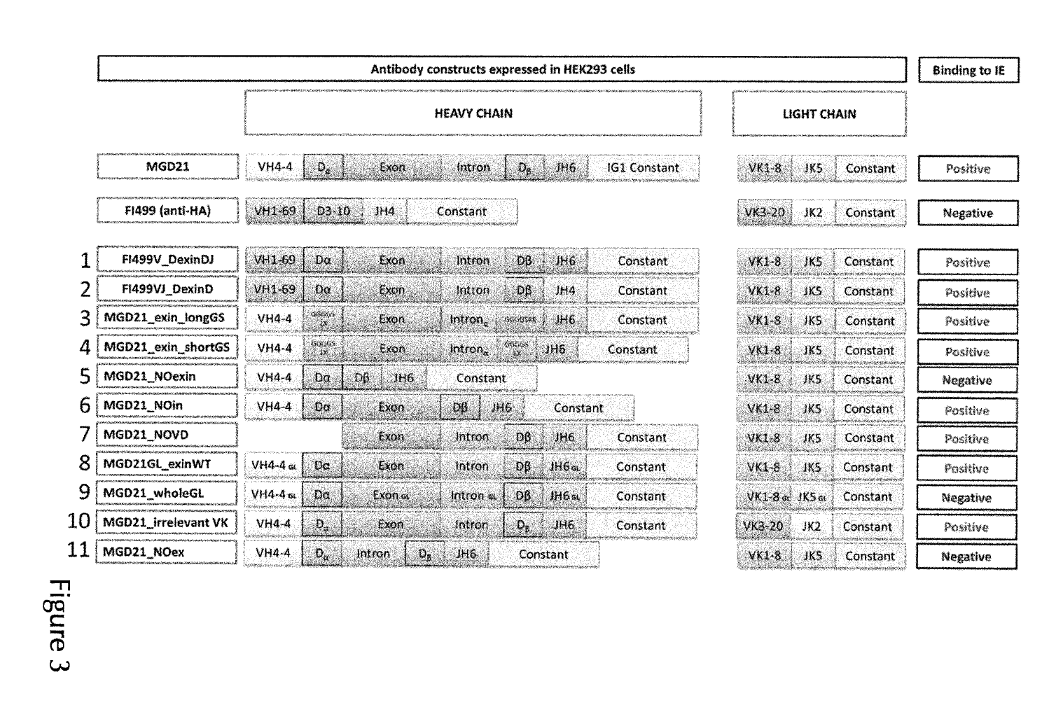

The following variants were produced, which are shown schematically in FIG. 3 (all the constructs have the same full complete constant region as antibody MGD21 as described herein and differ only in the heavy chain, while the light chain is not modified; the constructs are finally expressed as monoclonal antibodies (H+L chain)):
1. “F1499V_DexinDJ” is formed by (in this order from N- to C-terminus): the expression product of a V (variable) gene segment of a heavy chain variable region of FI499 (“VH1-69”), the expression product of a first D (Diversity) gene segment element of a heavy chain variable region of MGD21 (“D_{α}”); the mutated LAIR-1 fragment (“Exon”); the expression product of a LAIR-1 intron fragment (“Intron”); the expression product of a second D (Diversity) gene segment element of a heavy chain variable region of MGD21 (“D_{β}”); the expression product of a J (Joining) gene segment element of a heavy chain variable region of MGD21 (“JH6”); the expression product of a C (constant) gene segment of a heavy chain constant region (IgG1 isotype); and on a separate chain: the expression product of a V (variable) gene segment of a light chain variable region of MGD21 (“VK1-8”) and the expression product of a J (Joining) gene segment element of a light chain variable region of MGD21 (“JK5”); the expression product of a C (constant) gene segment of a light chain constant region.
2. “FI1499VJ_DexinD” is formed by (in this order from N- to C-terminus): the expression product of a V (variable) gene segment of a heavy chain variable region of FI499 (“VH1-69”), the expression product of a first D (Diversity) gene segment element of a heavy chain variable region of MGD21 (“D_{α}”); the mutated LAIR-1 fragment (“Exon”); the expression product of a LAIR-1 intron fragment (“Intron”); the expression product of a second D (Diversity) gene segment element of a heavy chain variable region of MGD21 (“D_{β}”); the expression product of a J (Joining) gene segment element of a heavy chain variable region of FI499 (“JH4”); the expression product of a C (constant) gene segment of a heavy chain constant region (IgG1 isotype); and on a separate chain: the expression product of a V (variable) gene segment of a light chain variable region of MGD21 (“VK1-8”) and the expression product of a J (Joining) gene segment element of a light chain variable region of MGD21 (“JK5”); the expression product of a C (constant) gene segment of a light chain constant region.
3. “MGD21_exin_longGS” is formed by (in this order from N- to C-terminus): the expression product of a V (variable) gene segment of a heavy chain variable region of MGD21 (“VH4-4”); the expression product of a 10-amino-acid linker (“GGGGS 2X”); the mutated LAIR-1 fragment (“Exon”); the expression product of a LAIR-1 intron fragment (“Introna”); the expression product of a 20-amino-acid linker (“GGGGS 4X”); the expression product of a J (Joining) gene segment element of a heavy chain variable region of MGD21 (“JH6”);

the expression product of a C (constant) gene segment of a heavy chain constant region (IgG1 isotype); and on a separate chain: the expression product of a V (variable) gene segment of a light chain variable region of MGD21 (“VK1-8”) and the expression product of a J (Joining) gene segment element of a light chain variable region of MGD21 (“JK5”); the expression product of a C (constant) gene segment of a light chain constant region.
4. “MGD21_exin_shortGS” is formed by (in this order from N- to C-terminus): the expression product of a V (variable) gene segment of a heavy chain variable region of MGD21 (“VH4-4”); the expression product of a 5-amino-acid linker (“GGGGS 1 X”); the mutated LAIR-1 fragment (“Exon”); the expression product of a LAIR-1 intron fragment (“Introna”); the expression product of a 5-amino-acid linker (“GGGGS 1X”); the expression product of a J (Joining) gene segment element of a heavy chain variable region of MGD21 (“JH6”); the expression product of a C (constant) gene segment of a heavy chain constant region (IgG1 isotype); and on a separate chain: the expression product of a V (variable) gene segment of a light chain variable region of MGD21 (“VK1-8”) and the expression product of a J (Joining) gene segment element of a light chain variable region of MGD21 (“JK5”); the expression product of a C (constant) gene segment of a light chain constant region.
5. “MGD21_NOexin” is formed by (in this order from N- to C-terminus): the expression product of a V (variable) gene segment of a heavy chain variable region of MGD21 (“VH4-4”); the expression product of a first D (Diversity) gene segment element of a heavy chain variable region of MGD21 (“D_{α}”); the expression product of a second D (Diversity) gene segment element of a heavy chain variable region of MGD21 (“D_{β}”); the expression product of a J (Joining) gene segment element of a heavy chain variable region of MGD21 (“JH6”); the expression product of a C (constant) gene segment of a heavy chain constant region (IgG1 isotype); and on a separate chain: the expression product of a V (variable) gene segment of a light chain variable region of MGD21 (“VK1-8”) and the expression product of a J (Joining) gene segment element of a light chain variable region of MGD21 (“JK5”); the expression product of a C (constant) gene segment of a light chain constant region.
6. “MGD21_NOin” is formed by (in this order from N- to C-terminus): the expression product of a V (variable) gene segment of a heavy chain variable region of MGD21 (“VH4-4”); the expression product of a first D (Diversity) gene segment element of a heavy chain variable region of MGD21 (“D_{α}”); the mutated LAIR-1 fragment (“Exon”); the expression product of a second D (Diversity) gene segment element of a heavy chain variable region of MGD21 (“D_{β}”); the expression product of a J (Joining) gene segment element of a heavy chain variable region of MGD21 (“JH6”); the expression product of a C (constant) gene segment of a heavy chain constant region (IgG1 isotype); and on a separate chain: the expression product of a V (variable) gene segment of a light chain variable region of MGD21 (“VK1-8”) and the expression product of a J (Joining) gene segment element of a light chain variable region of MGD21 (“JK5”); the expression product of a C (constant) gene segment of a light chain constant region.
7. “MGD21_NOVD” is formed by (in this order from N- to C-terminus): the mutated LAIR-1 fragment (“Exon”); the expression product of a LAIR-1 intron fragment (“Intron”); the expression product of a second D (Diversity) gene segment element of a heavy chain variable region of MGD21 (“D_{β}”); the expression product of a J (Joining) gene segment element of a heavy chain variable region of MGD21 (“JH6”); the expression product of a C (constant) gene segment of a heavy chain constant region (IgG1 isotype); and on a separate chain: the expression product of a V (variable) gene segment of a light chain variable region of MGD21 (“VK1-8”) and the expression product of a J (Joining) gene segment element of a light chain variable region of MGD21 (“JK5”); the expression product of a C (constant) gene segment of a light chain constant region.

8. “MGD21GL_exinWT” is formed by (in this order from N- to C-terminus): the expression product of an unmutated V (variable) gene segment of a heavy chain variable region of MGD21 (“VH4-4 GL”); the expression product of a first D (Diversity) gene segment element of a heavy chain variable region of MGD21 (“D_{α}”); the mutated LAIR-1 fragment (“Exon”); the expression product of a LAIR-1 intron fragment (“Intron”); the expression product of a second D (Diversity) gene segment element of a heavy chain variable region of MGD21 (“D_{β}”); the expression product of an unmutated J (Joining) gene segment element of a heavy chain variable region of MGD21 (“JH6 GL”); the expression product of a C (constant) gene segment of a heavy chain constant region (IgG1 isotype); and on a separate chain: the expression product of a V (variable) gene segment of a light chain variable region of MGD21 (“VK1-8”) and the expression product of a J (Joining) gene segment element of a light chain variable region of MGD21 (“JK5”); the expression product of a C (constant) gene segment of a light chain constant region.
9. “MGD21_wholeGL” is formed by (in this order from N- to C-terminus): the expression product of an unmutated V (variable) gene segment of a heavy chain variable region of MGD21 (“VH4-4 GL”); the expression product of a first D (Diversity) gene segment element of a heavy chain variable region of MGD21 (“D_{α}”); the unmutated LAIR-1 fragment (“Exon GL”); the expression product of a unmutated LAIR-1 intron fragment (“Intron GL”); the expression product of a second D (Diversity) gene segment element of a heavy chain variable region of MGD21 (“D_{β}”); the expression product of a J (Joining) gene segment element of a unmutated heavy chain variable region of MGD21 (“JH6 GL”); the expression product of a C (constant) gene segment of a heavy chain constant region (IgG1 isotype); and on a separate chain: the expression product of an unmutated V (variable) gene segment of a light chain variable region of MGD21 (“VK1-8 GL”) and the expression product of an unmutated J (Joining) gene segment element of a light chain variable region of MGD21 (“JK5 GL”); the expression product of a C (constant) gene segment of a light chain constant region.
10. “MGD21 _irrelevant VK” is formed by (in this order from N- to C-terminus): the expression product of a V (variable) gene segment of a heavy chain variable region of MGD21 (“VH4-4”); the expression product of a first D (Diversity) gene segment element of a heavy chain variable region of MGD21 (“D_{α}”); the mutated LAIR-1 fragment (“Exon”); the expression product of a LAIR-1 intron fragment (“Intron”); the expression product of a second D (Diversity) gene segment element of a heavy chain variable region of MGD21 (“D_{β}”); the expression product of a J (Joining) gene segment element of a heavy chain variable region of MGD21 (1H6″); the expression product of a C (constant) gene segment of a heavy chain constant region (IgG1 isotype); and on a separate chain: the expression product of a V (variable) gene segment of a light chain variable region of FI499 (“VK3-20”) and the expression product of a J (Joining) gene segment element of a light chain variable region of F1499 (“JK2”); the expression product of a C (constant) gene segment of a light chain constant region.
11. “MGD21_NOex” is formed by (in this order from N- to C-terminus): the expression product of a V (variable) gene segment of a heavy chain variable region of MGD21 (“VH4-4”); the expression product of a first D (Diversity) gene segment element of a heavy chain variable region of MGD21 (“D_{α}”); the expression product of a LAIR-1 intron fragment (“Intron”); the expression product of a second D (Diversity) gene segment element of a heavy chain variable region of MGD21 (“D_{β}”); the expression product of a J (Joining) gene segment element of a heavy chain variable region of MGD21 (1H6″); the expression product of a C (constant) gene segment of a heavy chain constant region (IgG1 isotype); and on a separate chain: the expression product of a V (variable) gene segment of a light chain variable region of MGD21 (“VK1-8”) and the expression product of a J (Joining) gene segment element of a light chain variable region of MGD21 (“JK5”); the expression product of a C (constant) gene segment of a light chain constant region.

Table 9 below provides amino acid and nucleic acid sequences of the heavy chain variable regions of the constructs described above (Example 3).

TABLE 9
Sequences and Seq IDs of constructs

| | | |
|---|---|---|
| SEQ | | |
| ID | | |
| NO | Description | Sequence* |

Heavy chain variable regions

| | | |
|---|---|---|
| 594 | FI499V_DexinDJ | QVQPVQSGAEVKEPGSSVKVSCKTSGGLIRKSAVSWVRQAP |
| | aa | GQGLEWMGGISALENTKDYAEKFQGRLTITADESTATAYMEL |
| | | SSLTSEDTAIYYCATASPLKSQRDTDLPRPSISAEPGTVIPLGSHV |
| | | TFVCRGPVGVQTFRLERERNYLYSDTEDVSQTSPSESEARFRID |
| | | SVNAGNAGLFRCIYYKSRKWSEQSDYLELVVKGEDVTWALSQ |
| | | SQDDPRACPQGELPISTDIYYVDVWGNGTTVTVSS |
| 595 | FI499V_DexinDJ | CAGGTGCAGCCCGTCCAGTCTGGAGCAGAGGTGAAGGA |
| | nucl | ACCTGGCAGCTCCGTGAAGGTCTCTTGCAAAACAAGTGG |
| | | CGGGCTGATCCGCAAAAGTGCCGTGTCATGGGTCCGACA |
| | | GGCTCCTGGACAGGGACTGGAATGGATGGGAGGCATCA |
| | | GCGCACTGTTCAACACTAAGGACTACGCCGAAAAATTTCA |
| | | GGGCCGGCTGACTATTACCGCCGATGAGAGTACAGCCAC |
| | | TGCTTATATGGAACTGTCTAGTCTGACCAGCGAGGACACA |
| | | GCTATCTACTATTGCGCAACCGCCTCACCACTGAAGTCCC |
| | | AGAGAGACACCGACCTGCCAAGACCTTCCATCTCTGCAG |
| | | AACCTGGCACAGTGATTCCACTGGGGTCCCACGTGACTTT |
| | | CGTCTGTAGGGGACCAGTGGGCGTCCAGACCTTTCGCCT |
| | | GGAGCGGGAAAGAAATTACCTGTATTCCGACACTGAGGA |
| | | CGTGAGCCAGACCAGTCCCTCAGAGAGCGAAGCTAGGTT |
| | | CCGCATCGATTCCGTGAACGCTGGGAATGCAGGACTGTT |
| | | TAGATGCATCTACTATAAGTCTAGGAAATGGAGCGAGCA |
| | | GTCCGACTACCTGGAACTGGTGGTCAAAGGGGAGGATG |
| | | TGACATGGGCTCTGTCCCAGTCTCAGGACGATCCAAGAG |
| | | CATGTCCCCAGGGCGAGCTGCCCATCTCTACTGACATCTA |
| | | CTATGTGGATGTCTGGGGCAACGGGACCACAGTGACCGT |
| | | CTCAAGC |
| 596 | FI499VJ_DexinD | QVQPVQSGAEVKEPGSSVKVSCKTSGGLIRKSAVSWVRQAP |
| | aa | GQGLEWMGGISALFNTKDYAEKFQGRLTITADESTATAYMEL |
| | | SSLTSEDTAIYYCATASPLKSQRDTDLPRPSISAEPGTVIPLGSHV |
| | | TFVCRGPVGVQTFRLERERNYLYSDTEDVSQTSPSESEARFRID |
| | | SVNAGNAGLFRCIYYKSRKWSEQSDYLELVVKGEDVTWALSQ |
| | | SQDDPRACPQGELPISTDIFDYWGQGTLVTVSS |
| 597 | FI499VJ_DexinD | CAGGTGCAGCCCGTCCAGTCTGGAGCAGAGGTGAAGGA |
| | nucl | ACCTGGCAGCTCCGTGAAGGTCTCTTGCAAAACAAGTGG |
| | | CGGGCTGATCCGCAAAAGTGCCGTGTCATGGGTCCGACA |
| | | GGCTCCTGGACAGGGACTGGAATGGATGGGAGGCATCA |
| | | GCGCACTGTTCAACACTAAGGACTACGCCGAAAAATTTCA |
| | | GGGCCGGCTGACCATTACAGCCGATGAGAGTACTGCCAC |
| | | CGCTTATATGGAACTGTCTAGTCTGACCAGCGAGGACAC |
| | | AGCTATCTACTATTGCGCAACCGCCTCACCACTGAAGTCC |
| | | CAGAGAGACACCGACCTGCCAAGACCTTCCATCTCTGCA |
| | | GAACCTGGCACAGTGATTCCACTGGGGTCCCACGTGACT |
| | | TTCGTCTGTAGGGGACCAGTGGGCGTCCAGACCTTTCGC |
| | | CTGGAGCGGGAAAGAAATTACCTGTATTCCGACACTGAG |
| | | GACGTGAGCCAGACCAGTCCCTCAGAGAGCGAAGCTAG |
| | | GTTCCGCATCGATTCCGTGAACGCTGGGAATGCAGGACT |
| | | GTTTAGATGCATCTACTATAAGTCTAGGAAATGGAGCGAG |
| | | CAGTCCGACTACCTGGAACTGGTGGTCAAAGGGGAGGA |
| | | TGTGACTTGGGCTCTGTCCCAGTCTCAGGACGATCCAAG |
| | | AGCATGTCCCCAGGGCGAGCTGCCCATCTCTACCGACAT |
| | | TTTCGATTATTGGGGCCAGGGGACACTGGTGACTGTCTC |
| | | AAGC |
| 598 | MGD21_exin_lo | EVQLVETGPGLMKTSGTLSLTCAVSGDYVNTNRRWSWVRQ |
| | ngGS aa | APGKGLEWIGEVHQSGRTNYNPSLKSRVTISVDKSKNQFSLKV |
| | | DSVTAADTAVYYCARGGGGS |
| | | GGGGSDLPRPSISAEPGTVIPLGSHVTFVCRGPVGVQTFRLER |
| | | ERNYLYSDTEDVSQTSPSESEARFRIDSVNAGNAGLFRCIYYKS |
| | | RKWSEQSDYLELVVKGEDVTWALGGGGS GGGGS GGGGS |
| | | GGGGSYYVDVWGNGTTVTVSS |
| 599 | MGD21_exin_lo | GAAGTGCAGCTGGTGGAAACCGGCCCTGGACTGATGAA |
| | ngGS nucl | GACCTCTGGCACACTGAGTCTGACATGCGCTGTGAGTGG |
| | | GGACTACGTCAACACTAATCGGAGATGGTCTTGGGTGCG |
| | | ACAGGCACCAGGAAAAGGACTGGAGTGGATCGGGGAA |
| | | GTGCACCAGAGCGGAAGGACCAACTATAATCCTAGCCTG |
| | | AAGTCCCGCGTGACAATTTCAGTCGATAAGAGCAAAAAC |
| | | CAGTTCTCCCTGAAAGTGGACTCTGTCACTGCCGCTGATA |
| | | CCGCAGTGTACTATTGTGCCAGAGGCGGGGGAGGCTCT |
| | | GGGGGAGGCGGGAGTGACCTGCCCAGGCCTAGCATCTC |
| | | CGCTGAACCAGGGACTGTGATTCCCCTGGGATCTCACGT |
| | | GACCTTCGTCTGCAGAGGCCCTGTGGGGGTCCAGACATT |
| | | TCGCCTGGAGCGGGAAAGAAACTACCTGTATTCTGACAC |
| | | CGAGGATGTGAGTCAGACATCTCCCAGTGAGTCAGAAGC |
| | | AAGGTTCCGCATCGATTCCGTCAACGCCGGAAATGCTGG |
| | | CCTGTTTCGATGTATCTACTATAAGAGCCGGAAATGGAGC |
| | | GAGCAGTCCGACTACCTGGAACTGGTGGTCAAGGGCGA |
| | | GGATGTGACCTGGGCCCTGGGCGGGGGAGGCTCTGGG |
| | | GGAGGCGGGAGTGGAGGCGGGGGATCAGGTGGAGGC |
| | | GGGTCGTACTATGTGGACGTGTGGGGCAACGGGACCAC |
| | | AGTGACCGTCAGCTCC |
| 600 | MGD21_exin_short | EVQLVETGPGLMKTSGTLSLTCAVSGDYVNTNRRWSWVRQ |
| | GS aa | APGKGLEWIGEVHQSGRTNYNPSLKSRVTISVDKSKNQFSLKV |
| | | DSVTAADTAVYYCARGGGGS |
| | | DLPRPSISAEPGTVIPLGSHVTFVCRGPVGVQTFRLERERNYLY |
| | | SDTEDVSQTSPSESEARFRIDSVNAGNAGLFRCIYYKSRKWSE |
| | | QSDYLELVVKGEDVTWALGGGGS YYVDVWGNGTTVTVSS |
| 601 | MGD21_exin_short | GAAGTGCAGCTGGTGGAAACCGGCCCTGGACTGATGAA |
| | GS nucl | GACCTCTGGCACACTGAGTCTGACATGCGCTGTGAGTGG |
| | | GGACTACGTCAACACTAATCGGAGATGGTCTTGGGTGCG |
| | | ACAGGCACCAGGAAAAGGACTGGAGTGGATCGGGGAA |
| | | GTGCACCAGAGCGGAAGGACCAACTATAATCCTAGCCTG |
| | | AAGTCCCGCGTGACAATTTCAGTCGATAAGAGCAAAAAC |
| | | CAGTTCTCCCTGAAAGTGGACTCTGTCACTGCCGCTGATA |
| | | CCGCAGTGTACTATTGTGCCAGAGGGGGAGGCGGGAGT |
| | | GACCTGCCCAGGCCTAGCATCTCCGCTGAACCAGGGACT |
| | | GTGATTCCCCTGGGATCTCACGTGACCTTCGTCTGCAGAG |
| | | GCCCTGTGGGGGTCCAGACATTTCGCCTGGAGCGGGAA |
| | | AGAAACTACCTGTATTCTGACACCGAGGATGTGAGTCAG |
| | | ACATCTCCCAGTGAGTCAGAAGCAAGGTTCCGCATCGATT |
| | | CCGTCAACGCCGGAAATGCTGGCCTGTTTCGATGTATCTA |
| | | CTATAAGAGCCGGAAATGGAGCGAGCAGTCCGACTACCT |
| | | GGAACTGGTGGTCAAGGGCGAGGATGTGACCTGGGCCC |
| | | TGGGAGGCGGGGGATCATACTATGTGGACGTGTGGGGC |
| | | AACGGGACCACAGTGACCGTCAGCTCC |
| 602 | MGD21_NOexin | EVQLVETGPGLMKTSGTLSLTCAVSGDYVNTNRRWSWVRQ |
| | aa | APGKGLEWIGEVHQSGRTNYNPSLKSRVTISVDKSKNQFSLKV |
| | | DSVTAADTAVYYCARASPLKSQRDTGELPISTDIYYVDVWGN |
| | | GTTVTVSS |
| 603 | MGD21_NOexin | GAAGTGCAGCTGGTGGAAACCGGCCCTGGACTGATGAA |
| | nucl | GACTTCAGGAACCCTGAGCCTGACTTGTGCCGTGAGCGG |
| | | CGACTACGTCAACACCAATCGGAGATGGAGTTGGGTGCG |
| | | GCAGGCACCAGGAAAAGGCCTGGAGTGGATCGGCGAA |
| | | GTGCACCAGTCTGGGCGAACAAACTATAATCCCTCTCTGA |
| | | AGAGTAGAGTGACTATTTCCGTGGACAAGTCTAAAAACCA |
| | | GTTCAGCCTGAAAGTGGACTCCGTCACAGCCGCTGATAC |
| | | TGCCGTGTACTATTGTGCAAGGGCCAGTCCCCTGAAGTC |
| | | ACAGCGCGATACCGGGGAGCTGCCTATCAGCACAGACAT |
| | | CTACTATGTGGATGTCTGGGGGAATGGAACCACAGTGAC |
| | | AGTCAGCTCC |
| 604 | MGD21_NOin | EVQLVETGPGLMKTSGTLSLTCAVSGDYVNTNRRWSWVRQ |
| | aa | APGKGLEWIGEVHQSGRTNYNPSLKSRVTISVDKSKNQFSLKV |
| | | DSVTAADTAVYYCARASPLKSQRDTDLPRPSISAEPGTVIPLGS |
| | | HVTFVCRGPVGVQTFRLERERNYLYSDTEDVSQTSPSESEARF |
| | | RIDSVNAGNAGLFRCIYYKSRKWSEQSDYLELVVKGELPISTDI |
| | | YYVDVWGNGTTVTVSS |
| 605 | MGD21_NOin | GAGGTGCAGCTGGTCGAAACCGGCCCAGGGCTGATGAA |
| | nucl | GACTTCCGGAACCCTGTCTCTGACATGCGCCGTGTCCGG |
| | | GGACTACGTCAACACTAATCGGAGATGGTCTTGGGTGAG |
| | | GCAGGCTCCTGGAAAAGGCCTGGAGTGGATCGGGGAAG |
| | | TGCACCAGTCCGGACGGACCAACTATAATCCATCTCTGAA |
| | | GAGTAGAGTGACAATTAGTGTCGATAAGTCAAAAAACCA |
| | | GTTCTCTCTGAAAGTGGACAGTGTCACAGCCGCTGATACT |
| | | GCAGTGTACTATTGTGCAAGAGCAAGCCCCCTGAAGTCC |
| | | CAGAGAGACACCGACCTGCCCAGGCCTTCTATCAGTGCT |
| | | GAACCAGGCACTGTGATTCCCCTGGGGTCTCATGTGACC |
| | | TTCGTCTGTAGAGGCCCCGTGGGAGTCCAGACTTTTCGC |
| | | CTGGAGAGGGAACGCAATTACCTGTATTCAGACACCGAG |
| | | GATGTGAGCCAGACATCACCTAGCGAGTCCGAAGCCCGA |
| | | TTCCGGATCGACAGTGTGAACGCTGGAAATGCAGGCCTG |
| | | TTTCGCTGTATCTACTATAAGAGCCGAAAATGGTCAGAGC |
| | | AGAGCGATTACCTGGAACTGGTGGTCAAAGGCGAGCTG |
| | | CCTATCAGCACTGACATCTACTATGTGGATGTCTGGGGGA |
| | | ACGGAACCACAGTGACCGTCAGCTCC |
| 606 | MGD21_NOVD | DLPRPSISAEPGTVIPLGSHVTFVCRGPVGVQTFRLERERNYLY |
| | aa | SDTEDVSQTSPSESEARFRIDSVNAGNAGLFRCIYYKSRKWSE |
| | | QSDYLELVVKGEDVTWALSQSQDDPRACPQGELPISTDIYYV |
| | | DVWGNGTTVTVSS |
| 607 | MGD21_NOVD | GACCTGCCACGACCATCTATTTCCGCCGAACCTGGGACT |
| | nucl | GTCATTCCTCTGGGGAGCCACGTCACATTTGTCTGCCGG |
| | | GGACCTGTCGGGGTGCAGACTTTCCGGCTGGAGCGGGA |
| | | AAGAAACTACCTGTATTCTGACACCGAAGATGTGAGTCAG |
| | | ACAAGCCCATCCGAGTCTGAAGCTAGGTTCCGCATCGAC |
| | | TCCGTCAACGCCGGCAATGCTGGGCTGTTTCGATGCATCT |
| | | ACTATAAGAGCAGAAAATGGAGCGAGCAGTCCGACTACC |
| | | TGGAACTGGTGGTCAAGGGAGAGGATGTCACCTGGGCA |
| | | CTGAGTCAGTCACAGGACGATCCCCGGGCCTGTCCTCAG |
| | | GGCGAGCTGCCCATCAGCACTGATATCTACTATGTGGAT |
| | | GTCTGGGGGAATGGCACTACTGTGACCGTCTCAAGC |
| 608 | MGD21GL_exin | QVQLQESGPGLVKPSGTLSLTCAVSGGSISSSNWWSWVRQP |
| | WT aa | PGKGLEWIGEIYHSGSTNYNPSLKSRVTISVDKSKNQFSLKLSS |
| | | VTAADTAVYYCARASPLKSQRDTDLPRPSISAEPGTVIPLGSHV |
| | | TFVCRGPVGVQTFRLERERNYLYSDTEDVSQTSPSESEARFRID |
| | | SVNAGNAGLFRCIYYKSRKWSEQSDYLELVVKGEDVTWALSQ |
| | | SQDDPRACPQGELPISTDIYYMDVWGKGTTVTVSS |
| 609 | MGD21GL_exin | CAGGTCCAGCTGCAGGAAAGCGGCCCAGGACTGGTGAA |
| | WT nucl | GCCTAGCGGAACACTGAGTCTGACTTGTGCCGTGAGCGG |
| | | AGGGAGCATCAGCTCCTCTAACTGGTGGTCTTGGGTGAG |
| | | GCAGCCCCCTGGCAAGGGACTGGAGTGGATCGGCGAAA |
| | | TCTACCACAGCGGGTCCACCAACTATAATCCTTCACTGAA |
| | | GAGCCGCGTGACAATCAGTGTGGACAAGTCAAAAAATCA |
| | | GTTCAGCCTGAAACTGAGTTCAGTGACCGCCGCTGATAC |
| | | AGCAGTCTACTATTGCGCACGGGCCAGCCCACTGAAATC |
| | | CCAGCGAGACACTGATCTGCCACGGCCCTCTATCAGTGC |
| | | TGAACCCGGAACAGTGATTCCTCTGGGCTCCCATGTGACT |
| | | TTCGTCTGTCGCGGACCAGTGGGCGTCCAGACCTTTCGA |
| | | CTGGAGCGGGAAAGAAACTACCTGTATTCTGACACTGAG |
| | | GATGTGAGTCAGACCTCACCCAGCGAGTCCGAAGCCAG |
| | | GTTCCGCATCGACAGCGTCAACGCTGGGAATGCAGGACT |
| | | GTTTAGATGCATCTACTATAAGTCCAGGAAATGGTCCGAG |
| | | CAGTCTGACTACCTGGAACTGGTGGTCAAGGGGGAGGA |
| | | TGTGACATGGGCCCTGTCTCAGAGTCAGGACGATCCTAG |
| | | AGCTTGTCCACAGGGCGAGCTGCCCATTTCAACCGATATC |
| | | TATTACATGGATGTCTGGGGCAAGGGCACCACCGTGACC |
| | | GTGAGCAGC |
| 610 | MGD21_wholeGL | QVQLQESGPGLVKPSGTLSLTCAVSGGSISSSNWWSWVRQP |
| | aa | PGKGLEWIGEIYHSGSTNYNPSLKSRVTISVDKSKNQFSLKLSS |
| | | VTAADTAVYYCARASPLKSQRDTEDLPRPSISAEPGTVIPLGSH |
| | | VTFVCRGPVGVQTERLERESRSTYNDTEDVSQASPSESEARFRI |
| | | DSVSEGNAGPYRCIYYKPPKWSEQSDYLELLVKGEDVTWALP |
| | | QSQLDPRACPQGELPISTDIYYMDVWGKGTTVTVSS |
| 611 | MGD21_wholeGL | CAGGTGCAGCTGCAGGAAAGCGGACCAGGCCTGGTCAA |
| | nucl | GCCCTCAGGCACTCTGAGCCTGACCTGCGCTGTGAGTGG |
| | | CGGGTCAATCAGCTCCTCTAATTGGTGGTCCTGGGTGAG |
| | | GCAGCCCCCTGGGAAAGGACTGGAGTGGATCGGCGAAA |
| | | TCTACCACTCTGGGAGTACAAACTATAATCCCAGCCTGAA |
| | | GTCCCGCGTGACTATTTCCGTGGACAAGTCTAAAAATCAG |
| | | TTCAGCCTGAAACTGAGTTCAGTGACAGCCGCTGATACTG |
| | | CAGTCTACTATTGCGCACGAGCCAGTCCTCTGAAGTCCCA |
| | | GCGGGACACTGAGGACCTGCCTAGACCATCAATCAGCGC |
| | | CGAGCCTGGAACTGTGATTCCACTGGGCTCTCATGTGAC |
| | | CTTCGTCTGTAGAGGACCAGTGGGAGTCCAGACCTTCCG |
| | | GCTGGAGAGAGAATCCCGATCTACCTACAACGACACAGA |
| | | AGATGTGAGCCAGGCTAGTCCATCAGAGAGCGAAGCAC |
| | | GGTTTAGAATCGACTCCGTGTCTGAGGGGAATGCCGGAC |
| | | CCTACAGATGCATCTACTATAAGCCACCCAAATGGTCTGA |
| | | GCAGAGTGACTATCTGGAACTGCTGGTGAAAGGAGAGG |
| | | ATGTCACCTGGGCACTGCCTCAGTCTCAGCTGGACCCCA |
| | | GAGCTTGTCCTCAGGGAGAGCTGCCTATCAGCACCGACA |
| | | TCTACTATATGGACGTGTGGGGCAAAGGGACCACAGTGA |
| | | CAGTCAGCTCCGCGTCGACTTCGCA |
| 612 | MGD21_NOex | EVQLVETGPGLMKTSGTLSLTCAVSGDYVNTNRRWSWVRQ |
| | aa | APGKGLEWIGEVHQSGRTNYNPSLKSRVTISVDKSKNQFSLKV |
| | | DSVTAADTAVYYCARASPLKSQRDTGEDVTWALSQSQDDPR |
| | | ACPQGELPISTDIYYVDVWGNGTTVTVSS |
| 613 | MGD21_NOex | GAAGTGCAGCTGGTGGAAACCGGCCCTGGACTGATGAA |
| | nucl | GACTTCCGGAACCCTGTCTCTGACTTGCGCCGTGTCTGGC |
| | | GACTACGTCAACACCAATCGGAGATGGAGCTGGGTGCG |
| | | GCAGGCTCCAGGAAAAGGCCTGGAGTGGATCGGCGAAG |
| | | TGCACCAGTCCGGGCGAACAAACTATAATCCCTCACTGAA |
| | | GAGCAGAGTGACTATTAGTGTCGATAAGTCAAAAAACCA |
| | | GTTCTCTCTGAAAGTGGACAGTGTCACAGCCGCTGATACT |
| | | GCCGTGTACTATTGCGCAAGGGCCAGCCCTCTGAAGTCC |
| | | CAGAGAGACACCGGGGAGGATGTGACATGGGCTCTGTC |
| | | TCAGAGTCAGGACGATCCCCGGGCATGTCCTCAGGGCG |
| | | AACTGCCAATCAGCACCGACATCTACTATGTGGATGTCTG |
| | | GGGGAATGGAACCACAGTGACAGTCAGCTCC |

### Example 4

### Identification of the Mutated LAIR-1 Exon as the Only Element Required for MGD21 mAb Binding to P. falciparum-Infected Erythrocytes (IEs)

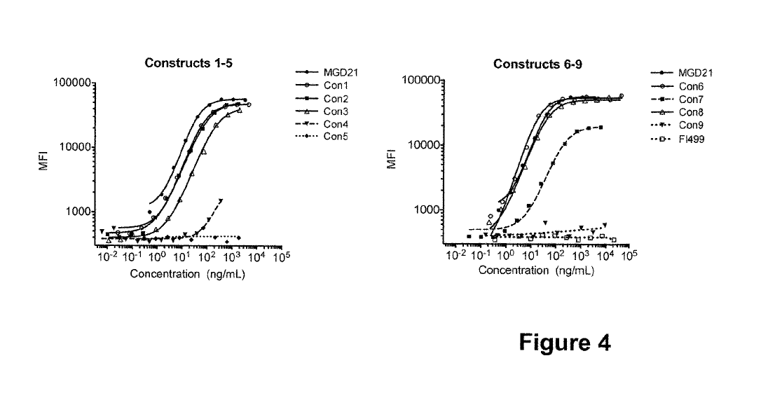

The 10 antibody variants constructed in Example 3 as well as the antibody MGD21 (cf. Examples 1 and 2) and the antibody FI499 (control: irrelevant antibody reactive to influenza virus hemagglutinin, HA) were expressed in HEK 293 cells and tested for their capacity to stain IEs as described in Example 1. Briefly, IEs are stained with SYBR Green I dye (DNA) to discriminate them from uninfected erythrocytes used as control. The antibody variants are added on top of IEs and binding of specific antibodies to IEs is detected using a secondary-anti-human IgG (Fc -specific) antibody. The binding data are shown in FIG. 4. Most constructs show binding to IEs, with the exception of those constructus wherein the exon is either not present or is in the original genomic form (Con5/“MGD21_NOexin”, Con9/“MGD21_wholeGL” and Con11 “MGD21_NOex”). The results indicate that the only element required for binding to IEs is the mutated LAIR-1 exon.

### Example 5

### Construction of Ig Fusion Proteins Comprising the Mutated LAIR-1 Fragment

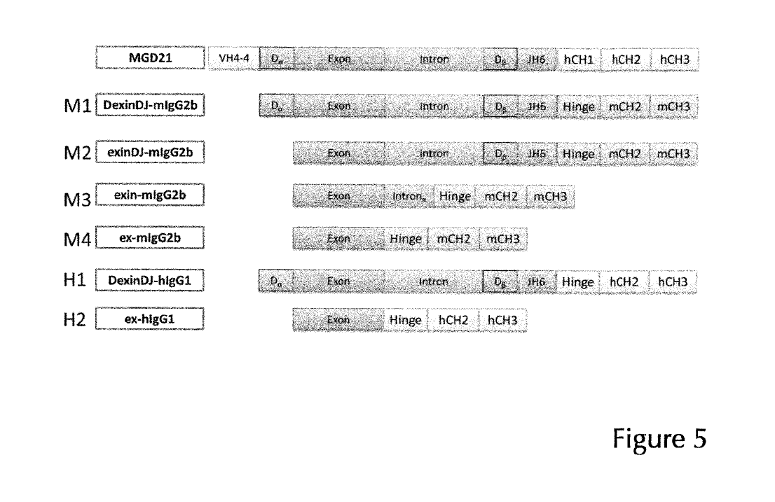

To investigate whether the mutated LAIR-1 exon alone is sufficient to bind to IEs, six different Ig fusion proteins comprising the mutated LAIR-1 fragment were constructed by inserting:
(a) the mutated LAIR-1 exon, preferably according to SEQ ID NO: 84 or a functional sequence variant thereof;
(b) optionally, one or more further elements (intron segments) of LAIR-1, preferably corresponding to such elements of the antibody MGD21 as shown in FIG. 5 and;
(c) optionally, one or more different elements of a heavy chain variable region of an IgG-type antibody, preferably of the antibody MGD21,

into a plasmid designed for expression of mouse IgG2b fusion proteins (pINFUSE-rnIgG2b-Fc2 by Invivogen) or human IgG1 fusion proteins (pINFUSE-hIgG1-Fc2 by Invivogen). Preferred sequences for the constant regions (hinge region and CH2 and CH3 domains) of mouse IgG2b fusion proteins comprise or consist of a sequence according to SEQ ID NO: 614 (amino acid) or SEQ ID NO: 615 (nucleic acid), or functional sequence variants thereof. Preferred sequences for the constant regions (hinge region and CH2 and CH3 domains) of human IgG1 fusion proteins comprise or consist of a sequence according to SEQ ID NO: 616 (amino acid) or SEQ ID NO: 617 (nucleic acid), or functional sequence variants thereof. Preferably, the mutated LAIR-1 fragment (“Exon”) in the following Ig fusion proteins comprises or consists of an amino acid sequence according to SEQ ID NO: 83 or a functional sequence variant thereof.

The different fusion proteins are shown schematically in FIG. 5 in comparison to the antibody MGD21 and described in the following:
1. M1 (also referred to as “DexinDJ-mIgG2b”) is formed by (in this order from N- to C-terminus): the expression product of a first D (Diversity) gene segment element of a heavy chain variable region of an IgG-type antibody, preferably of MGD21 (“D_{α}”); the mutated LAIR-1 fragment (“Exon”); the expression product of a LAIR-1 intron fragment (“Intron”); the expression product of a second D (Diversity) gene segment element of a heavy chain variable region of an IgG-type antibody, preferably of MGD21 (“Dr”); the expression product of a J (Joining) gene segment element of a heavy chain variable region of an IgG-type antibody, preferably of MGD21 (“JH6”); followed by a hinge region and CH2 and CH3 domains from mouse IgG2b.
   An exemplary variable region of such an M1 fusion protein, which is particularly preferred, comprises or consists of an amino acid sequence according to SEQ ID NO: 618 or according to a functional sequence variant thereof, which may preferably be encoded by a nucleic acid sequence according to SEQ ID NO: 619 or by a functional sequence variant thereof. More preferably, a complete M1 fusion protein comprises or consists of an amino acid sequence according to SEQ ID NO: 620 or according to a functional sequence variant thereof, which may preferably be encoded by a nucleic acid sequence according to SEQ ID NO: 621 or by a functional sequence variant thereof.
2. M2 (also referred to as “exinDJ-mIgG2b”) is formed by (in this order from N- to C-terminus): the mutated LAIR-1 fragment (“Exon”); the expression product of a LAIR-1 intron fragment (“Intron”); the expression product of a second D (Diversity) gene segment element of a heavy chain variable region of an IgG-type antibody, preferably of MGD21 (“D_{β}”); the expression product of a J (Joining) gene segment element of a heavy chain variable region of an IgG-type antibody, preferably of MGD21 (“JH6”); followed by a hinge region and CH2 and CH3 domains from mouse IgG2b.
   An exemplary variable region of such an M2 fusion protein, which is particularly preferred, comprises or consists of an amino acid sequence according to SEQ ID NO: 624 or according to a functional sequence variant thereof, which may preferably be encoded by a nucleic acid sequence according to SEQ ID NO: 625 or by a functional sequence variant thereof. More preferably, a complete M2 fusion protein comprises or consists of an amino acid sequence according to SEQ ID NO: 626 or according to a functional sequence variant thereof, which may preferably be encoded by a nucleic acid sequence according to SEQ ID NO: 627 or by a functional sequence variant thereof.
3. M3 (also referred to as “exin-mIgG2b”) is formed by (in this order from N- to C-terminus): the mutated LAIR-1 fragment (“Exon”); the expression product of a partial LAIR-1 intron fragment (“Intron_{α}”); followed by a hinge region and CH2 and CH3 domains from mouse IgG2b.
   An exemplary variable region of such an M3 fusion protein, which is particularly preferred, comprises or consists of an amino acid sequence according to SEQ ID NO: 628 or according to a functional sequence variant thereof, which may preferably be encoded by a nucleic acid sequence according to SEQ ID NO: 629 or by a functional sequence variant thereof. More preferably, a complete M3 fusion protein comprises or consists of an amino acid sequence according to SEQ ID NO: 630 or according to a functional sequence variant thereof, which may preferably be encoded by a nucleic acid sequence according to SEQ ID NO: 631 or by a functional sequence variant thereof.
4. M4 (also referred to as “ex-mIgG2b”) is formed by (in this order from N- to C-terminus): the mutated LAIR-1 fragment (“Exon”); followed by a hinge region and CH2 and CH3 domains from mouse IgG2b.
   An exemplary variable region of such an M4 fusion protein, which is particularly preferred, comprises or consists of an amino acid sequence according to SEQ ID NO: 632 or according to a functional sequence variant thereof, which may preferably be encoded by a nucleic acid sequence according to SEQ ID NO: 633 or by a functional sequence variant thereof. More preferably, a complete M4 fusion protein comprises or consists of an amino acid sequence according to SEQ ID NO: 634 or according to a functional sequence variant thereof, which may preferably be encoded by a nucleic acid sequence according to SEQ ID NO: 635 or by a functional sequence variant thereof.
5. H1 (also referred to as “DexinDJ-hIgG1”) is formed by (in this order from N- to C-terminus): the expression product of a first D (Diversity) gene segment element of a heavy chain variable region of an IgG-type antibody, preferably of MGD21 (“D_{α}”); the mutated LAIR-1 fragment (“Exon”); the expression product of a LAIR-1 intron fragment (“Intron”); the expression product of a second D (Diversity) gene segment element of a heavy chain variable region of an IgG-type antibody, preferably of MGD21 (“D_{β}”); the expression product of a J (Joining) gene segment element of a heavy chain variable region of an IgG-type antibody, preferably of MGD21 (“JH6”); followed by a hinge region and CH2 and CH3 domains from human IgG1.
   An exemplary variable region of such an H1 fusion protein, which is particularly preferred, comprises or consists of an amino acid sequence according to SEQ ID NO: 618 or according to a functional sequence variant thereof, which may preferably be encoded by a nucleic acid sequence according to SEQ ID NO: 619 or by a functional sequence variant thereof. More preferably, a complete H1 fusion protein comprises or consists of an amino acid sequence according to SEQ ID NO: 622 or according to a functional sequence variant thereof, which may preferably be encoded by a nucleic acid sequence according to SEQ ID NO: 623 or by a functional sequence variant thereof.
6. H2 (also referred to as “ex-hIgG1”) is formed by (in this order from N- to C-terminus): the mutated LAIR-1 fragment (“Exon”); followed by a hinge region and CH2 and CH3 domains from human IgG1.
   An exemplary variable region of such an H2 fusion protein, which is particularly preferred, comprises or consists of an amino acid sequence according to SEQ ID NO: 632 or according to a functional sequence variant thereof, which may preferably be encoded by a nucleic acid sequence according to SEQ ID NO: 633 or by a functional sequence variant thereof. More preferably, a complete H2 fusion protein comprises or consists of an amino acid sequence according to SEQ ID NO: 636 or according to a functional sequence variant thereof, which may preferably be encoded by a nucleic acid sequence according to SEQ ID NO: 637 or by a functional sequence variant thereof.

Table 10 below shows the amino acid and nucleotide sequences of the antibody constructs of Example 5, whereby the constant chain sequences are identical for the mouse IgG2b-antibody constructs M1, M2, M3, and M4 (“mIgG2b”) and for the human IgG1-antibody constructs H1 and H2 (“hIgG1”).

TABLE 10
Sequences and Seq IDs of Ig fusion proteins

| | | |
|---|---|---|
| SEQ | | |
| ID | | |
| NO | Description | Sequence* |

Constant chains

| | | |
|---|---|---|
| 614 | mIgG2b aa | AMVRSPSGPISTINPCPPCKECHKCPAPNLEGGPSVFIFPPNIKD |
| | | VLMISLTPKVTCVVVDVSEDDPDVQISWFVNNVEVHTAQTQ |
| | | THREDYNSTIRVVSTLPIQHQDWMSGKEFKCKVNNKDLPSPIE |
| | | RTISKIKGLVRAPQVYILPPPAEQLSRKDVSLTCLVVGFNPGDIS |
| | | VEWTSNGHTEENYKDTAPVLDSDGSYFIYSKLNMKTSKWEKT |
| | | DSFSCNVRHEGLKNYYLKKTISRSPGK |
| 615 | mIgG2b nucl | GCCATGGTTAGATCTCCCAGCGGGCCCATTTCAACAATCA |
| | | ACCCCTGTCCTCCATGCAAGGAGTGTCACAAATGCCCAG |
| | | CTCCTAACCTCGAGGGTGGACCATCCGTCTTCATCTTCCC |
| | | TCCAAATATCAAGGATGTACTCATGATCTCCCTGACACCC |
| | | AAGGTCACGTGTGTGGTGGTGGATGTGAGCGAGGATGA |
| | | CCCAGACGTCCAGATCAGCTGGTTTGTGAACAACGTGGA |
| | | AGTACACACAGCTCAGACACAAACCCATAGAGAGGATTA |
| | | CAACAGTACTATCCGGGTGGTCAGCACCCTCCCCATCCA |
| | | GCACCAGGACTGGATGAGTGGCAAGGAGTTCAAATGCA |
| | | AGGTCAACAACAAAGACCTCCCATCACCCATCGAGAGAA |
| | | CCATCTCAAAAATTAAAGGGCTAGTCAGAGCTCCACAAGT |
| | | ATACATCTTGCCGCCACCAGCAGAGCAGTTGTCCAGGAA |
| | | AGATGTCAGTCTCACTTGCCTGGTCGTGGGCTTCAACCCT |
| | | GGAGACATCAGTGTGGAGTGGACCAGCAATGGGCATAC |
| | | AGAGGAGAACTACAAGGACACCGCACCAGTCCTGGACTC |
| | | TGACGGTTCTTACTTCATATATAGCAAGCTCAATATGAAAA |
| | | CAAGCAAGTGGGAGAAAACAGATTCCTTCTCATGCAACG |
| | | TGAGACACGAGGGTCTGAAAAATTACTACCTGAAGAAGA |
| | | CCATCTCCCGGTCTCCGGGTAAA |
| 616 | hIgG1 aa | AMVRSDKTHTCPPCPAPELLGGPSVFLFPPKPKDTLMISRTPEV |
| | | TCVVVDVSHEDPEVKFNWYVDGVEVHNAKTKPREEQYNSTY |
| | | RVVSVLTVLHQDWLNGKEYKCKVSNKALPAPIEKTISKAKGQP |
| | | REPQVYTLPPSRDELTKNQVSLTCLVKGFYPSDIAVEWESNGQ |
| | | PENNYKTTPPVLDSDGSFFLYSKLTVDKSRWQQGNVFSCSVM |
| | | HEALHNHYTQKSLSLSPGK |
| 617 | hIgG1 nucl | GCCATGGTTAGATCTGACAAAACTCACACATGCCCACCGT |
| | | GCCCAGCACCTGAACTCCTGGGGGGACCGTCAGTCTTCC |
| | | TCTTCCCCCCAAAACCCAAGGACACCCTCATGATCTCCCG |
| | | GACCCCTGAGGTCACATGCGTGGTGGTGGACGTGAGCC |
| | | ACGAAGACCCTGAGGTCAAGTTCAACTGGTACGTGGACG |
| | | GCGTGGAGGTGCATAATGCCAAGACAAAGCCGCGGGAG |
| | | GAGCAGTACAACAGCACGTACCGTGTGGTCAGCGTCCTC |
| | | ACCGTCCTGCACCAGGACTGGCTGAATGGCAAGGAGTAC |
| | | AAGTGCAAGGTCTCCAACAAAGCCCTCCCAGCCCCCATC |
| | | GAGAAAACCATCTCCAAAGCCAAAGGGCAGCCCCGAGA |
| | | ACCACAGGTGTACACCCTGCCCCCATCCCGGGATGAGCT |
| | | GACCAAGAACCAGGTCAGCCTGACCTGCCTGGTCAAAGG |
| | | CTTCTATCCCAGCGACATCGCCGTGGAGTGGGAGAGCAA |
| | | TGGGCAGCCGGAGAACAACTACAAGACCACGCCTCCCGT |
| | | GCTGGACTCCGACGGCTCCTTCTTCCTCTACAGCAAGCTC |
| | | ACCGTGGACAAGAGCAGGTGGCAGCAGGGGAACGTCTT |
| | | CTCATGCTCCGTGATGCATGAGGCTCTGCACAACCACTAC |
| | | ACGCAGAAGAGCCTCTCCCTGTCTCCGGGTAAA |

MGD21-DexinDJ-mIgG2b

| | | |
|---|---|---|
| 618 | DexinDJ variable | ASPLKSQRDTDLPRPSISAEPGTVIPLGSHVTFVCRGPVGVQTF |
| | part aa | RLERERNYLYSDTEDVSQTSPSESEARFRIDSVNAGNAGLFRCI |
| | | YYKSRKWSEQSDYLELVVKGEDVTWALSQSQDDPRACPQGE |
| | | LPISTDIYYVDVWGNGTTVTVSS |
| 619 | DexinDJ variable | gcgtctccactcaaatctcagagggacaccgatctgcccagaccctccatctcggctg |
| | part nucl | agccgggcaccgtgatccccctggggagccatgtgactacgtgtgccggggcccggt |
| | | tggggttcaaacattccgcctggagagggagaggaattatttatacagtgatactgaaga |
| | | tgtgtctcaaactagtccatctgagtcggaggccagattccgcattgactcagtaaatgc |
| | | aggcaatgccgggctttttcgctgcatctattacaagtcccgtaaatggtctgagcagag |
| | | tgactacctggagctggtggtgaaaggtgaggacgtcacctgggccctgtcccagtctc |
| | | aagacgaccctcgagcttgtccccagggggagctccccataagtaccgatatttacta |
| | | cgtggacgtctggggcaacgggaccacggtcaccgtctcctca |
| 620 | DexinDJ-mIgG2b | ASPLKSQRDTDLPRPSISAEPGTVIPLGSHVTFVCRGPVGVQTF |
| | complete | RLERERNYLYSDTEDVSQTSPSESEARFRIDSVNAGNAGLFRCI |
| | sequence aa | YYKSRKWSEQSDYLELVVKGEDVTWALSQSQDDPRACPQGE |
| | | LPISTDIYYVDVWGNGTTVTVSSAMVRSPSGPISTINPCPPCKE |
| | | CHKCPAPNLEGGPSVFIFPPNIKDVLMISLTPKVTCVVVDVSED |
| | | DPDVQISWFVNNVEVHTAQTQTHREDYNSTIRVVSTLPIQH |
| | | QDWMSGKEFKCKVNNKDLPSPIERTISKIKGLVRAPQVYILPPP |
| | | AEQLSRKDVSLTCLVVGFNPGDISVEWTSNGHTEENYKDTAP |
| | | VLDSDGSYFIYSKLNMKTSKWEKTDSFSCNVRHEGLKNYYLKK |
| | | TISRSPGK |
| 621 | DexinDJ-mIgG2b | gcgtctccactcaaatctcagagggacaccgatctgcccagaccctccatctcggctg |
| | complete | agccgggcaccgtgatccccctggggagccatgtgactttcgtgtgccggggcccggt |
| | sequence nucl | tggggttcaaacattccgcctggagagggagaggaattatttatacagtgatactgaaga |
| | | tgtgtctcaaactagtccatctgagtcggaggccagattccgcattgactcagtaaatgc |
| | | aggcaatgccgggctttttcgctgcatctattacaagtcccgtaaatggtctgagcagag |
| | | tgactacctggagctggtggtgaaaggtgaggacgtcacctgggccctgtcccagtctc |
| | | aagacgaccctcgagcttgtccccagggggagctccccataagtaccgatatttacta |
| | | cgtggacgtctggggcaacgggaccacggtcaccgtctcctcaGCCATGGTTA |
| | | GATCTCCCAGCGGGCCCATTTCAACAATCAACCCCTGTCC |
| | | TCCATGCAAGGAGTGTCACAAATGCCCAGCTCCTAACCTC |
| | | GAGGGTGGACCATCCGTCTTCATCTTCCCTCCAAATATCA |
| | | AGGATGTACTCATGATCTCCCTGACACCCAAGGTCACGTG |
| | | TGTGGTGGTGGATGTGAGCGAGGATGACCCAGACGTCC |
| | | AGATCAGCTGGTTTGTGAACAACGTGGAAGTACACACAG |
| | | CTCAGACACAAACCCATAGAGAGGATTACAACAGTACTAT |
| | | CCGGGTGGTCAGCACCCTCCCCATCCAGCACCAGGACTG |
| | | GATGAGTGGCAAGGAGTTCAAATGCAAGGTCAACAACAA |
| | | AGACCTCCCATCACCCATCGAGAGAACCATCTCAAAAATT |
| | | AAAGGGCTAGTCAGAGCTCCACAAGTATACATCTTGCCG |
| | | CCACCAGCAGAGCAGTTGTCCAGGAAAGATGTCAGTCTC |
| | | ACTTGCCTGGTCGTGGGCTTCAACCCTGGAGACATCAGT |
| | | GTGGAGTGGACCAGCAATGGGCATACAGAGGAGAACTA |
| | | CAAGGACACCGCACCAGTCCTGGACTCTGACGGTTCTTA |
| | | CTTCATATATAGCAAGCTCAATATGAAAACAAGCAAGTGG |
| | | GAGAAAACAGATTCCTTCTCATGCAACGTGAGACACGAG |
| | | GGTCTGAAAAATTACTACCTGAAGAAGACCATCTCCCGGT |
| | | CTCCGGGTAAA |
| 622 | DexinDJ-hIgG1 | ASPLKSQRDTDLPRPSISAEPGTVIPLGSHVTFVCRGPVGVQTF |
| | complete | RLERERNYLYSDTEDVSQTSPSESEARFRIDSVNAGNAGLFRCI |
| | sequence aa | YYKSRKWSEQSDYLELVVKGEDVTWALSQSQDDPRACPQGE |
| | | LPISTDIYYVDVWGNGTTVTVSSAMVRSDKTHTCPPCPAPELL |
| | | GGPSVFLFPPKPKDTLMISRTPEVTCVVVDVSHEDPEVKFNWY |
| | | VDGVEVHNAKTKPREEQYNSTYRVVSVLTVLHQDWLNGKEY |
| | | KCKVSNKALPAPIEKTISKAKGQPREPQVYTLPPSRDELTKNQV |
| | | SLICLVKGFYPSDIAVEWESNGQPENNYKTTPPVLDSDGSFEL |
| | | YSKLTVDKSRWQQGNVFSCSVMHEALHNHYTQKSLSLSPGK |
| 623 | DexinDJ-hIgG1 | gcgtctccactcaaatctcagagggacaccgatctgcccagaccctccatctcggctg |
| | complete | agccgggcaccgtgatccccctggggagccatgtgactttcgtgtgccggggcccggt |
| | sequence nucl | tggggttcaaacattccgcctggagagggagaggaattattlatacagtgatactgaaga |
| | | tgtgtctcaaactagtccatctgagtcggaggccagattccgcattgactcagtaaatgc |
| | | aggcaatgccgggctttttcgctgcatctattacaagtcccgtaaatggtctgagcagag |
| | | tgactacctggagctggtggtgaaaggtgaggacgtcacctgggccctgtcccagtctc |
| | | aagacgaccctcgagcttgtccccagggggagctccccataagtaccgatatttacta |
| | | cgtggacgtctggggcaacgggaccacggtcaccgtctcctcaGCCATGGTTA |
| | | GATCTGACAAAACTCACACATGCCCACCGTGCCCAGCAC |
| | | CTGAACTCCTGGGGGGACCGTCAGTCTTCCTCTTCCCCCC |
| | | AAAACCCAAGGACACCCTCATGATCTCCCGGACCCCTGA |
| | | GGTCACATGCGTGGTGGTGGACGTGAGCCACGAAGACC |
| | | CTGAGGTCAAGTTCAACTGGTACGTGGACGGCGTGGAG |
| | | GTGCATAATGCCAAGACAAAGCCGCGGGAGGAGCAGTA |
| | | CAACAGCACGTACCGTGTGGTCAGCGTCCTCACCGTCCT |
| | | GCACCAGGACTGGCTGAATGGCAAGGAGTACAAGTGCA |
| | | AGGTCTCCAACAAAGCCCTCCCAGCCCCCATCGAGAAAA |
| | | CCATCTCCAAAGCCAAAGGGCAGCCCCGAGAACCACAG |
| | | GTGTACACCCTGCCCCCATCCCGGGATGAGCTGACCAAG |
| | | AACCAGGTCAGCCTGACCTGCCTGGTCAAAGGCTTCTATC |
| | | CCAGCGACATCGCCGTGGAGTGGGAGAGCAATGGGCAG |
| | | CCGGAGAACAACTACAAGACCACGCCTCCCGTGCTGGAC |
| | | TCCGACGGCTCCTTCTTCCTCTACAGCAAGCTCACCGTGG |
| | | ACAAGAGCAGGTGGCAGCAGGGGAACGTCTTCTCATGCT |
| | | CCGTGATGCATGAGGCTCTGCACAACCACTACACGCAGA |
| | | AGAGCCTCTCCCTGTCTCCGGGTAAA |

MGD21-exinDJ-mIgG2b

| | | |
|---|---|---|
| 624 | exinDJ variable | DLPRPSISAEPGTVIPLGSHVTFVCRGPVGVQTFRLERERNYLY |
| | part aa | SDTEDVSQTSPSESEARFRIDSVNAGNAGLFRCIYYKSRKWSE |
| | | QSDYLELVVKGEDVTWALSQSQDDPRACPQGELPISTDIYYV |
| | | DVWGNGTTVTVSS |
| 625 | exinDJ variable | gatctgcccagaccctccatctcggctgagccgggcaccgtgatccccctggggagc |
| | part nucl | catgtgactttcgtgtgccggggcccggttggggttcaaacattccgcctggagaggga |
| | | gaggaattatttatacagtgatactgaagatgtgtctcaaactagtccatctgagtcggag |
| | | gccagattccgcattgactcagtaaatgcaggcaatgccgggctttttcgctgcatctatt |
| | | acaagtcccgtaaatggtctgagcagagtgactacctggagctggtggtgaaaggtga |
| | | ggacgtcacctgggccctgtcccagtctcaagacgaccctcgagcttgtccccaggg |
| | | ggagctccccataagtaccgatatttactacgtggacgtctggggcaacgggaccacg |
| | | gtcaccgtctcctca |
| 626 | exinDJ-mIgG2b | DLPRPSISAEPGTVIPLGSHVTFVCRGPVGVQTFRLERERNYLY |
| | complete | SDTEDVSQTSPSESEARFRIDSVNAGNAGLFRCIYYKSRKWSE |
| | sequence aa | QSDYLELVVKGEDVTWALSQSQDDPRACPQGELPISTDIYYV |
| | | DVWGNGTTVTVSSAMVRSDKTHTCPPCPAPELLGGPSVFLFP |
| | | PKPKDTLMISRTPEVTCVVVDVSHEDPEVKFNWYVDGVEVH |
| | | NAKTKPREEQYNSTYRVVSVLTVLHQDWLNGKEYKCKVSNK |
| | | ALPAPIEKTISKAKGQPREPQVYTLPPSRDELTKNQVSLTCLVK |
| | | GFYPSDIAVEWESNGQPENNYKTTPPVLDSDGSFFLYSKLTVD |
| | | KSRWQQGNVFSCSVMHEALHNHYTQKSLSLSPGK |
| 627 | exinDJ-mIgG2b | gatctgcccagaccctccatctcggctgagccgggcaccgtgatccccctggggagc |
| | complete | catgtgactttcgtgtgccggggcccggttggggttcaaacattccgcctggagaggga |
| | sequence nucl | gaggaattatttatacagtgatactgaagatgtgtctcaaactagtccatctgagtcggag |
| | | gccagattccgcattgactcagtaaatgcaggcaatgccgggctttttcgctgcatctatt |
| | | acaagtcccgtaaatggtctgagcagagtgactacctggagctggtggtgaaaggtga |
| | | ggacgtcacctgggccctgtcccagtctcaagacgaccctcgagcttgtccccaggg |
| | | ggagctccccataagtaccgatatttactacgtggacgtctggggcaacgggaccacg |
| | | gtcaccgtctcctcaGCCATGGTTAGATCTGACAAAACTCACACA |
| | | TGCCCACCGTGCCCAGCACCTGAACTCCTGGGGGGACC |
| | | GTCAGTCTTCCTCTTCCCCCCAAAACCCAAGGACACCCTC |
| | | ATGATCTCCCGGACCCCTGAGGTCACATGCGTGGTGGTG |
| | | GACGTGAGCCACGAAGACCCTGAGGTCAAGTTCAACTGG |
| | | TACGTGGACGGCGTGGAGGTGCATAATGCCAAGACAAA |
| | | GCCGCGGGAGGAGCAGTACAACAGCACGTACCGTGTGG |
| | | TCAGCGTCCTCACCGTCCTGCACCAGGACTGGCTGAATG |
| | | GCAAGGAGTACAAGTGCAAGGTCTCCAACAAAGCCCTCC |
| | | CAGCCCCCATCGAGAAAACCATCTCCAAAGCCAAAGGGC |
| | | AGCCCCGAGAACCACAGGTGTACACCCTGCCCCCATCCC |
| | | GGGATGAGCTGACCAAGAACCAGGTCAGCCTGACCTGC |
| | | CTGGTCAAAGGCTTCTATCCCAGCGACATCGCCGTGGAG |
| | | TGGGAGAGCAATGGGCAGCCGGAGAACAACTACAAGAC |
| | | CACGCCTCCCGTGCTGGACTCCGACGGCTCCTTCTTCCTC |
| | | TACAGCAAGCTCACCGTGGACAAGAGCAGGTGGCAGCA |
| | | GGGGAACGTCTTCTCATGCTCCGTGATGCATGAGGCTCT |
| | | GCACAACCACTACACGCAGAAGAGCCTCTCCCTGTCTCC |
| | | GGGTAAA |

MGD21-exin-mIgG2b

| | | |
|---|---|---|
| 628 | exin variable part | DLPRPSISAEPGTVIPLGSHVTFVCRGPVGVQTERLERERNYLY |
| | aa | SDTEDVSQTSPSESEARFRIDSVNAGNAGLFRCIYYKSRKWSE |
| | | QSDYLELVVKGEDVTWAL |
| 629 | exin variable part | gatctgcccagaccctccatctcggctgagccgggcaccgtgatccccctggggagc |
| | nucl | catgtgactttcgtgtgccggggcccggttggggttcaaacattccgcctggagaggga |
| | | gaggaattatttatacagtgatactgaagatgtgtctcaaactagtccatctgagtcggag |
| | | gccagattccgcattgactcagtaaatgcaggcaatgccgggctttttcgctgcatctatt |
| | | acaagtcccgtaaatggtctgagcagagtgactacctggagctggtggtgaaaggtga |
| | | ggacgtcacctgggccctg |
| 630 | exin-mIgG2b | DLPRPSISAEPGTVIPLGSHVTFVCRGPVGVQTERLERERNYLY |
| | complete | SDTEDVSQTSPSESEARFRIDSVNAGNAGLFRCIYYKSRKWSE |
| | sequence aa | QSDYLELVVKGEDVTWALAMVRSDKTHTCPPCPAPELLGGPS |
| | | VFLEPPKPKDTLMISRTPEVTCVVVDVSHEDPEVKFNWYVDG |
| | | VEVHNAKTKPREEQYNSTYRVVSVLTVLHQDWLNGKEYKCK |
| | | VSNKALPAPIEKTISKAKGQPREPQVYTLPPSRDELTKNQVSLT |
| | | CLVKGFYPSDIAVEWESNGQPENNYKTTPPVLDSDGSFFLYSK |
| | | LTVDKSRWQQGNVFSCSVMHEALHNHYTQKSLSLSPGK |
| 631 | exin-mIgG2b | gatctgcccagaccctccatctcggctgagccgggcaccgtgatccccctggggagc |
| | complete | catgtgactttcgtgtgccggggcccggttggggttcaaacattccgcctggagaggga |
| | sequence nucl | gaggaattatttatacagtgatactgaagatgtgtctcaaactagtccatctgagtcggag |
| | | gccagattccgcattgactcagtaaatgcaggcaatgccgggcatttttcgctgcatctatt |
| | | acaagtcccgtaaatggtctgagcagagtgactacctggagctggtggtgaaaggtga |
| | | ggacgtcacctgggccctgGCCATGGTTAGATCTGACAAAACTCA |
| | | CACATGCCCACCGTGCCCAGCACCTGAACTCCTGGGGGG |
| | | ACCGTCAGTCTTCCTCTTCCCCCCAAAACCCAAGGACACC |
| | | CTCATGATCTCCCGGACCCCTGAGGTCACATGCGTGGTG |
| | | GTGGACGTGAGCCACGAAGACCCTGAGGTCAAGTTCAAC |
| | | TGGTACGTGGACGGCGTGGAGGTGCATAATGCCAAGAC |
| | | AAAGCCGCGGGAGGAGCAGTACAACAGCACGTACCGTG |
| | | TGGTCAGCGTCCTCACCGTCCTGCACCAGGACTGGCTGA |
| | | ATGGCAAGGAGTACAAGTGCAAGGTCTCCAACAAAGCCC |
| | | TCCCAGCCCCCATCGAGAAAACCATCTCCAAAGCCAAAG |
| | | GGCAGCCCCGAGAACCACAGGTGTACACCCTGCCCCCAT |
| | | CCCGGGATGAGCTGACCAAGAACCAGGTCAGCCTGACCT |
| | | GCCTGGTCAAAGGCTTCTATCCCAGCGACATCGCCGTGG |
| | | AGTGGGAGAGCAATGGGCAGCCGGAGAACAACTACAAG |
| | | ACCACGCCTCCCGTGCTGGACTCCGACGGCTCCTTCTTCC |
| | | TCTACAGCAAGCTCACCGTGGACAAGAGCAGGTGGCAG |
| | | CAGGGGAACGTCTTCTCATGCTCCGTGATGCATGAGGCT |
| | | CTGCACAACCACTACACGCAGAAGAGCCTCTCCCTGTCTC |
| | | CGGGTAAA |

MGD21-ex-mIgG2b

| | | |
|---|---|---|
| 632 | exon variable | DLPRPSISAEPGTVIPLGSHVTFVCRGPVGVQTFRLERERNYLYSDTEDVSQTS |
| | part aa | PSESEARFRIDSVNAGNAGLFRCIYYKSRKWSEQSDYLELVVK |
| 633 | exon variable | gatctgcccagaccctccatctcggctgagccgggcaccgtgatccccctggggagc |
| | part nucl | catgtgactttcgtgtgccggggcccggttggggttcaaacattccgcctggagaggga |
| | | gaggaattatttatacagtgatactgaagatgtgtctcaaactagtccatctgagtcggag |
| | | gccagattccgcattgactcagtaaatgcaggcaatgccgggctttttcgctgcatctatt |
| | | acaagtcccgtaaatggtctgagcagagtgactacctggagctggtggtgaaa |
| 634 | ex-mIgG2b | DLPRPSISAEPGTVIPLGSHVTFVCRGPVGVQTFRLERERNYLY |
| | complete | SDTEDVSQTSPSESEARFRIDSVNAGNAGLFRCIYYKSRKWSE |
| | sequence aa | QSDYLELVVKAMVRSPSGPISTINPCPPCKECHKCPAPNLEGG |
| | | PSVFIFPPNIKDVLMISLTPKVTCVVVDVSEDDPDVQISWFVN |
| | | NVEVHTAQTQTHREDYNSTIRVVSTLPIQHQDWMSGKEFKC |
| | | KVNNKDLPSPIERTISKIKGLVRAPQVYILPPPAEQLSRKDVSLT |
| | | CLVVGFNPGDISVEWTSNGHTEENYKDTAPVLDSDGSYFIYS |
| | | KLNMKTSKWEKTDSFSCNVRHEGLKNYYLKKTISRSPGK |
| 635 | ex-mIgG2b | gatctgcccagaccctccatctcggctgagccgggcaccgtgatccccctggggagc |
| | complete | catgtgactttcgtgtgccggggcccggttggggttcaaacattccgcctggagaggga |
| | sequence nucl | gaggaattatttatacagtgatactgaagatgtgtctcaaactagtccatctgagtcggag |
| | | gccagattccgcattgactcagtaaatgcaggcaatgccgggctttttcgctgcatctatt |
| | | acaagtcccgtaaatggtctgagcagagtgactacctggagctggtggtgaaaGCC |
| | | ATGGTTAGATCTCCCAGCGGGCCCATTTCAACAATCAACC |
| | | CCTGTCCTCCATGCAAGGAGTGTCACAAATGCCCAGCTCC |
| | | TAACCTCGAGGGTGGACCATCCGTCTTCATCTTCCCTCCA |
| | | AATATCAAGGATGTACTCATGATCTCCCTGACACCCAAGG |
| | | TCACGTGTGTGGTGGTGGATGTGAGCGAGGATGACCCA |
| | | GACGTCCAGATCAGCTGGTTTGTGAACAACGTGGAAGTA |
| | | CACACAGCTCAGACACAAACCCATAGAGAGGATTACAAC |
| | | AGATACTATCCGGGTGGTCAGCACCCTCCCCATCCAGCAC |
| | | CAGGACTGGATGAGTGGCAAGGAGTTCAAATGCAAGGT |
| | | CAACAACAAAGACCTCCCATCACCCATCGAGAGAACCATC |
| | | TCAAAAATTAAAGGGCTAGTCAGAGCTCCACAAGTATACA |
| | | TCTTGCCGCCACCAGCAGAGCAGTTGTCCAGGAAAGATG |
| | | TCAGTCTCACTTGCCTGGTCGTGGGCTTCAACCCTGGAGA |
| | | CATCAGTGTGGAGTGGACCAGCAATGGGCATACAGAGG |
| | | AGAACTACAAGGACACCGCACCAGTCCTGGACTCTGACG |
| | | GTTCTTACTTCATATATAGCAAGCTCAATATGAAAACAAGC |
| | | AAGTGGGAGAAAACAGATTCCTTCTCATGCAACGTGAGA |
| | | CACGAGGGTCTGAAAAATTACTACCTGAAGAAGACCATCT |
| | | CCCGGTCTCCGGGTAAA |
| 636 | ex-hIgG1 | DLPRPSISAEPGTVIPLGSHVTFVCRGPVGVQTFRLERERNYLY |
| | complete | SDTEDVSQTSPSESEARFRIDSVNAGNAGLFRCIYYKSRKWSE |
| | sequence aa | QSDYLELVVKAMVRSDKTHTCPPCPAPELLGGPSVFLFPPKPK |
| | | DTLMISRTPEVTCVVVDVSHEDPEVKFNWYVDGVEVHNAKT |
| | | KPREEQYNSTYRVVSVLTVLHQDWLNGKEYKCKVSNKALPAP |
| | | IEKTISKAKGQPREPQVYTLPPSRDELTKNQVSLTCLVKGFYPS |
| | | DIAVEWESNGQPENNYKTTPPVLDSDGSFFLYSKLTVDKSRW |
| | | QQGNVFSCSVMHEALHNHYTQKSLSLSPGK |
| 637 | ex-hIgG1 | gatctgcccagaccctccatctcggctgagccgggcaccgtgatccccctggggagc |
| | complete | catgtgactttcgtgtgccggggcccggttggggttcaaacattccgcctggagaggga |
| | sequence nucl | gaggaattatttatacagtgatactgaagatgtgtctcaaactagtccatctgagtcggag |
| | | gccagattccgcattgactcagtaaatgcaggcaatgccgggctttttcgctgcatctatt |
| | | acaagtcccgtaaatggtctgagcagagtgactacctggagctggtggtgaaaGCC |
| | | ATGGTTAGATCTGACAAAACTCACACATGCCCACCGTGCC |
| | | CAGCACCTGAACTCCTGGGGGGACCGTCAGTCTTCCTCTT |
| | | CCCCCCAAAACCCAAGGACACCCTCATGATCTCCCGGAC |
| | | CCCTGAGGTCACATGCGTGGTGGTGGACGTGAGCCACG |
| | | AAGACCCTGAGGTCAAGTTCAACTGGTACGTGGACGGCG |
| | | TGGAGGTGCATAATGCCAAGACAAAGCCGCGGGAGGAG |
| | | CAGTACAACAGCACGTACCGTGTGGTCAGCGTCCTCACC |
| | | GTCCTGCACCAGGACTGGCTGAATGGCAAGGAGTACAA |
| | | GTGCAAGGTCTCCAACAAAGCCCTCCCAGCCCCCATCGA |
| | | GAAAACCATCTCCAAAGCCAAAGGGCAGCCCCGAGAACC |
| | | ACAGGTGTACACCCTGCCCCCATCCCGGGATGAGCTGAC |
| | | CAAGAACCAGGTCAGCCTGACCTGCCTGGTCAAAGGCTT |
| | | CTATCCCAGCGACATCGCCGTGGAGTGGGAGAGCAATG |
| | | GGCAGCCGGAGAACAACTACAAGACCACGCCTCCCGTG |
| | | CTGGACTCCGACGGCTCCTTCTTCCTCTACAGCAAGCTCA |
| | | CCGTGGACAAGAGCAGGTGGCAGCAGGGGAACGTCTTC |
| | | TCATGCTCCGTGATGCATGAGGCTCTGCACAACCACTACA |
| | | CGCAGAAGAGCCTCTCCCTGTCTCCGGGTAAA |

### Example 6

### Ig Fusion Proteins Comprising the Mutated LAIR-1 Fragment Bind to IEs

The four exemplary mouse IgG2b fusion proteins constructed in Example 5 (i.e. one of each type: M1, M2, M3, and M4), which were consisting of amino acid sequences as outlined for the “complete fusion protein”, respectively, were used to investigate whether the mutated LAIR-1 fragment is sufficient to bind to infected erythrocytes (IEs). To this end, HEK 293 cells were transfected with the fusion proteins only and supernatants were collected and tested for binding to IEs as described in Example 1. Briefly, IEs are stained with SYBR Green I dye (DNA) to discriminate them from uninfected erythrocytes used as control. The surnatants are added on top of IEs and binding of fusion proteins to IEs is detected using a secondary-anti-human or anti-mouse IgG (Fc -specific) antibody.

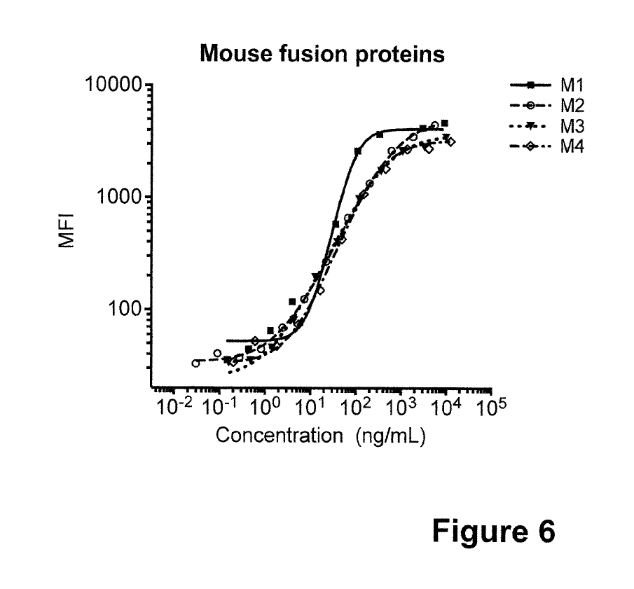

All fusion proteins were found to bind to infected erythrocytes (FIG. 6). These results identify the mutated LAIR-1 fragment as a unique domain that binds to P. falciparum-infected erythrocytes.

### Example 7

### Antibodies and Ig Fusion Proteins Efficiently Opsonize and Agglutinate P. falciparum-Infected Erythrocytes

To investigate the potential therapeutic impact of selected broadly reactive antibodies of Example 1 and of the Ig fusion proteins constructed in Example 5, i.e. whether these antibodies/fusion proteins could opsonize infected erythrocytes and thus mediate their phagocytosis and destruction by mononuclear phagocytes, their capacity to opsonize infected erythrocytes was measured.

To this end, P. falciparum (3D7) were stained with DAPI and mixed with different concentrations of the two exemplary human IgG1 fusion proteins constructed in Example 5 (i.e. one of each type: H1 and H2), which were consisting of amino acid sequences as outlined for the “complete fusion protein”, respectively. Thereafter, they were incubated with human monocytes at 37° C. for 1 hour.

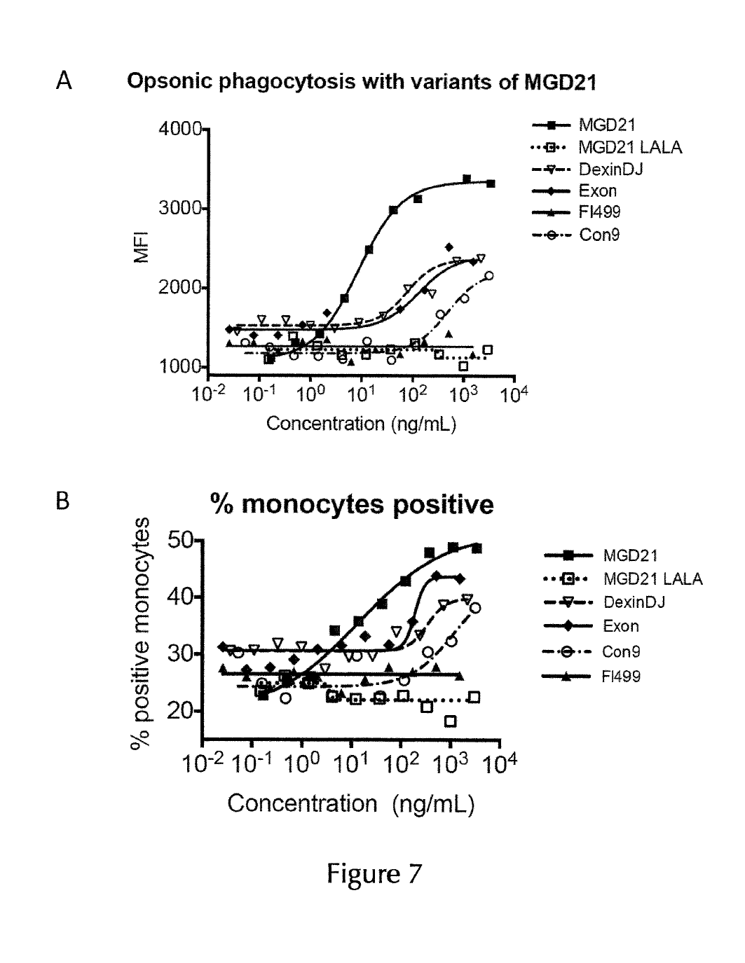

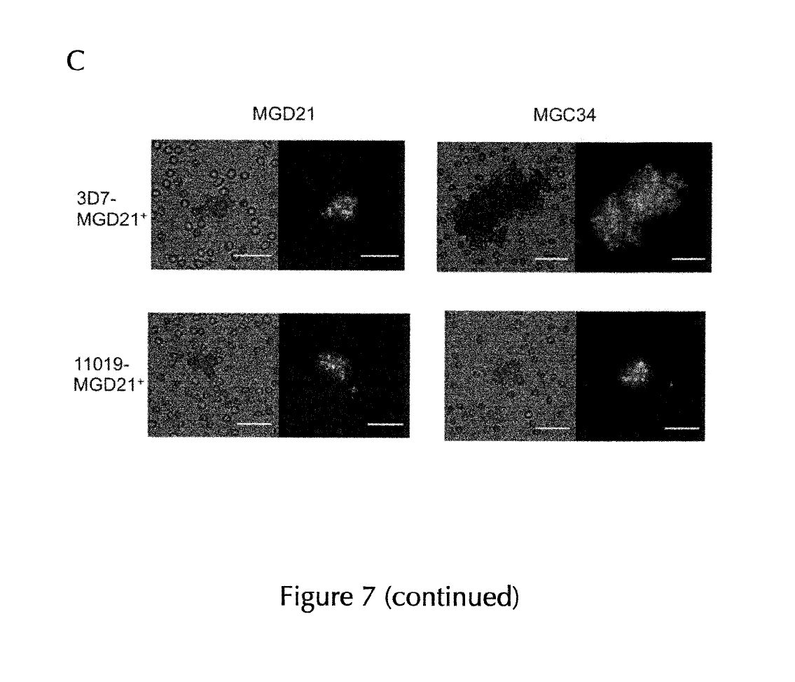

Thereafter, monocytes were stained with anti-CD14-APC to measure the fraction of monocytes that contained parasites. The results are shown in FIG. 7 with FIG. 7A showing the MFI (mean fluorecnce intensity) of DAPI and FIG. 7B showing the percentage of DAPI-positive monocytes calculated in CD14-positive populations.

The results demonstrate that low concentrations of the two exemplary human IgG1 fusion proteins constructed in Example 5 can efficiently opsonize infected erythrocytes. These findings indicate that the Ig fusion proteins constructed in Example 5 can potently mediate phagocytosis and destruction of infected erythrocytes in vivo.

Finally, it was tested whether the antibodies MGD21 and MGC34 were able to agglutinate erythrocytes infected with P. falciparum 3D7 or the Kenyan P. falciparum isolate 11019. As shown in FIG. 7C MGD21, as well as MGC34, could agglutinate erythrocytes infected with 3D7 or the Kenyan isolate 11019.

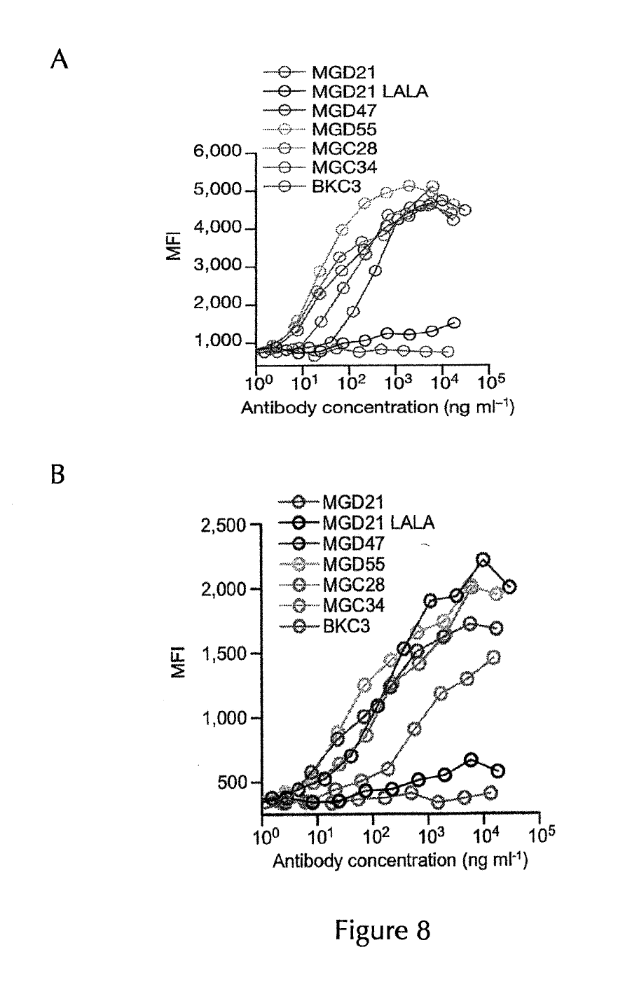

Next, P. falciparum (3D7 or 11019) were stained with DAPI and mixed with different concentrations of the five broadly reactive antibodies described in Table 2 and Example 1 (i.e. one of each type: MGD21, MGD47, MGD55, MGC28 and MGC34). BKC3 was used as control. Thereafter, they were incubated with human monocytes at 37° C. for 1 hour and, then, monocytes were stained with anti-CD14-APC to measure the fraction of monocytes that contained parasites. The results are shown in FIG. 8 with FIG. 8A showing the MFI (mean fluorecnce intensity) of DAPI in 3D7 and FIG. 8B showing the MFI (mean fluorecnce intensity) of DAPI in 11019-MGD21⁺IEs. Results show that low concentrations of all five antibodies tested constructed (MGD21, MGD47, MGD55, MGC28 and MGC34; cf. Table 2) can efficiently opsonize infected erythrocytes, whereas MGD21 LALA and BKC3 controls show no effect. These findings indicate that the broadly reactive antibodies can potently mediate phagocytosis and destruction of infected erythrocytes in vivo.

### Example 8

### A Model of the Mutated LAIR-1 Fragment: Somatic Mutations in the LAIR-1 Fragment are Critical Both for Binding IEs and Losing Binding to Collagen

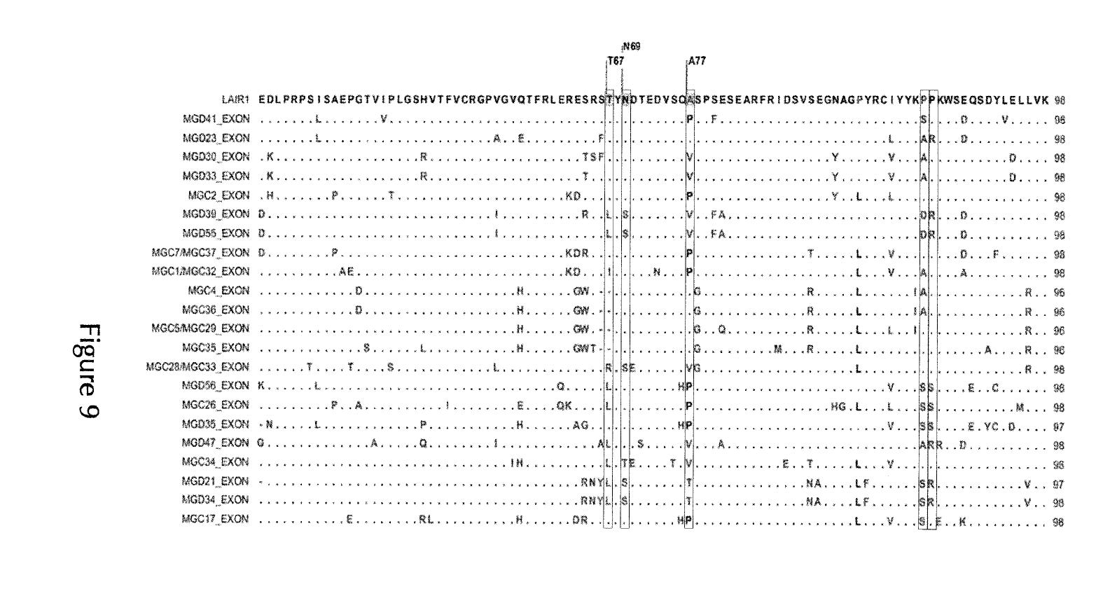

The mutated LAIR-1 fragment of the antibodies of Example 1 has a sequence homology ranging from 84% to 96% with the amino acids 24 to 121 of native human LAIR-1 (SEQ ID NO: 14; for example: MGD53_exon=96%; MGC2_exon=91%; MGD21_exon=86%; MGD35_exon=84%). FIG. 9 shows an alignment of the mutated LAIR-1 exon of the human monoclonal antibodies of Example 1 (cf. SEQ ID NOs 61, 63, 65, 67, 69, 71, 73, 75, 77, 79, 81, 83, 85, 87, 89, 91, 93, 95, 97, 99, 101 and 103—Table 1) with amino acids 24 to 121 of native human LAIR-1 (SEQ ID NO: 14).

From the human monoclonal antibodies of Example 1 those antibodies were selected, which most strongly bind to the most of the IEs infected with different P. falciparum strains (“broadest” binding to IEs). These were MGD21, MGD34, MGD39, MGD47, and MGD55 (cf. Table 7 of Example 1). An alignment of the amino acid sequences of the LAIR-1 exon fragment of these antibodies, i.e. amino acid sequences according to SEQ ID NOs: 83, 91, 95, 99 and 101 with an exemplary genomic LAIR-1 sequence, revealed five mutated residues, which are crucial to increase the affinity and the breadth of binding to P. falciparum-IEs. The same five mutated residues were also found to be important for losing binding to collagen that is the natural ligand of the native LAIR-1 receptor (see Example 9). The five crucial positions are T67, N69, A77, P106 and P107 and are shown in frames in FIG. 9.

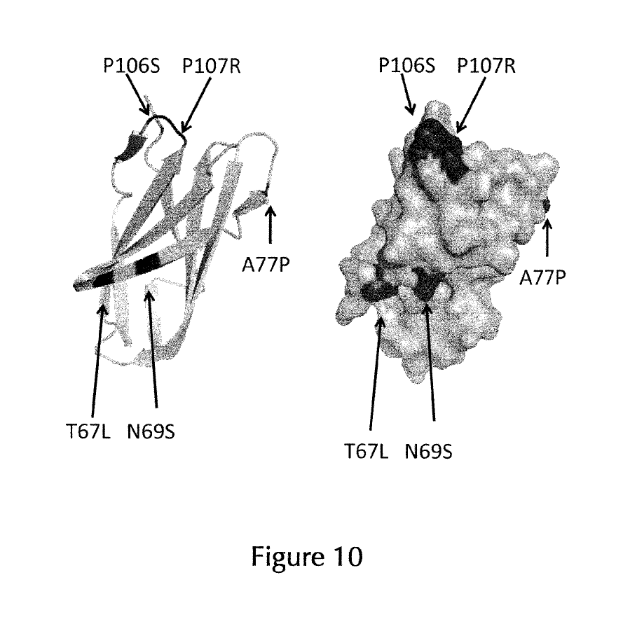

The mutated LAIR-1 fragment according to the present invention was modelled based on a crystal structure of native LAIR-1 extracellular domain (residues: 24 to 121) (FIG. 10; for the crystal structure of native LAIR-1 see MMDB ID: 78950, PDB ID: 3KGR). According to the crystal structure of LAIR-1, at least one of the following five residues must be mutated to lose collagen binding and to gain binding to infected erythrocytes (positions are defined in respect to the amino acid sequence of native human LAIR-1):

T67, N69, A77, P106, and P107 (FIG. 10).

Preferred mutations are shown below in Table 11, with T67L, N69S, A77T, P106S, and P107R being the most preferred mutations for each of the five positions.

TABLE 11
preferred mutations for each of the five
positions in the mutated LAIR-1 fragment.

| Position | Mutation |
|---|---|
| T67 | T67L, T67G, T67I, T67R, T67K |
| N69 | N69S, N69T |
| A77 | A77T, A77P, A77V |
| P106 | P106S, P106A, P106D |
| P107 | P107R, P107S |

### Example 9

### Identification of Mutations of LAIR1 Fragment that are Crucial for Binding to P. falciparum-IE

To identify which of the five mutations are crucial for binding to IEs, fusion proteins comprising the LAIR-1 fragment, which was either unmutated (SEQ ID NO: 14) or carrying one or more of the following five mutations: T67L (“L”); N69S (“Si”); A77T (“T”); P106S (“S2”); and P107R (“R”), were produced. The principal structure of these fusion proteins (i.e. except for the mutated LAIR-1 fragment) is identical to that of “H2” of Example 5 as described above (also referred to as “ex-hIgG1”). While in the construct “H2” of Example 5 (also referred to as “ex-hIgG1”) the mutated LAIR-1 exon of the antibody MGD21 was used (SEQ ID NO: 83), the present constructs are instead based on the native human LAIR-1 fragment (amino acids 24-121; SEQ ID NO: 14) and differ from that (i.e. from SEQ ID NO: 14) only in one or more of the following five mutations: T67L (“L”); N695 (“S1”); A77T (“T”); P106S (“S2”); and P107R (“R”).

Table 12 shows SEQ ID and sequences of the different fusion proteins.

TABLE 12
Sequences and Seq ID NOs of the LAIR-1 Ig fusion protein
constructs of Example 9, whereby only the sequences of the
(mutated) LAIR-1 fragment are shown. Mutations in comparison
to native human LAIR-1 (SEQ ID NO: 14) are shown underlined
in the amino acid sequence.

| SEQ | | |
|---|---|---|
| ID | | |
| NO | Description | Sequence* |
| 14 | LAIR1ex aa | EDLPRPSISAEPGTVIPLGSHVTFVCRGPVGVQTFRLERESRSTY |
| | | NDTEDVSQASPSESEARFRIDSVSEGNAGPYRCIYYKPPKWSE |
| | | QSDYLELLVK |
| 638 | LAIR1ex nucl | GAGGACCTGCCAAGACCCAGCATCTCCGCAGAACCTGG |
| | | GACTGTGATTCCACTGGGCTCCCACGTGACCTTCGTCTGC |
| | | AGAGGCCCCGTGGGAGTCCAGACCTTCCGGCTGGAGCG |
| | | CGAATCTCGAAGTACCTACAACGACACAGAGGACGTGAG |
| | | CCAGGCCTCACCCAGCGAGTCCGAAGCTCGGTTCAGAAT |
| | | CGACTCTGTCAGTGAAGGAAATGCCGGCCCTTACAGATG |
| | | CATCTACTATAAGCCCCCTAAATGGTCAGAGCAGAGCGAT |
| | | TATCTGGAACTGCTGGTGAAG |
| 23 | LAIR1ex + L aa | EDLPRPSISAEPGTVIPLGSHVTFVCRGPVGVQTFRLERESRSLY |
| | | NDTEDVSQASPSESEARFRIDSVSEGNAGPYRCIYYKPPKWSE |
| | | QSDYLELLVK |
| 24 | LAIR1ex + L nucl | GAGGACCTGCCAAGACCCAGCATCTCCGCAGAACCTGG |
| | | GACCGTGATTCCACTGGGCTCCCACGTGACATTCGTCTGC |
| | | AGAGGCCCCGTGGGAGTCCAGACTTTTAGGCTGGAGCG |
| | | CGAATCTCGAAGTCTGTACAACGACACAGAGGACGTGAG |
| | | CCAGGCCTCACCAAGCGAGTCCGAAGCTCGGTTCAGAAT |
| | | CGACTCTGTCAGTGAAGGAAATGCCGGCCCTTACAGATG |
| | | CATCTACTATAAGCCCCCTAAATGGTCAGAGCAGAGCGAT |
| | | TATCTGGAACTGCTGGTGAAG |
| 25 | LAIR1ex + LR aa | EDLPRPSISAEPGTVIPLGSHVTFVCRGPVGVQTFRLERESRSLY |
| | | NDTEDVSQASPSESEARFRIDSVSEGNAGPYRCIYYKPRKWSE |
| | | QSDYLELLVK |
| 26 | LAIR1ex + LR nucl | GAGGACCTGCCCCGCCCTAGCATCTCCGCAGAACCAGG |
| | | GACCGTGATTCCCCTGGGCTCCCACGTGACATTCGTCTGC |
| | | AGGGGCCCCGTGGGAGTCCAGACTTTTAGGCTGGAGCG |
| | | CGAATCTCGAAGTCTGTACAACGACACCGAGGACGTGAG |
| | | CCAGGCCTCACCTAGCGAGTCCGAAGCTCGGTTCAGAAT |
| | | CGACTCTGTCAGTGAAGGAAATGCCGGCCCTTACAGATG |
| | | CATCTACTATAAGCCAAGAAAATGGTCAGAGCAGAGCGA |
| | | TTATCTGGAACTGCTGGTGAAG |
| 27 | LAIR1ex + LS1 aa | EDLPRPSISAEPGTVIPLGSHVTFVCRGPVGVQTFRLERESRSLY |
| | | SDTEDVSQASPSESEARFRIDSVSEGNAGPYRCIYYKPPKWSEQ |
| | | SDYLELLVK |
| 28 | LAIR1ex + LS1 | GAGGACCTGCCAAGACCCAGCATCTCCGCAGAACCTGG |
| | nucl | GACCGTGATTCCACTGGGCTCCCACGTGACATTCGTCTGC |
| | | AGAGGCCCCGTGGGAGTCCAGACTTTTAGGCTGGAGCG |
| | | CGAATCTCGAAGTCTGTACTCCGACACAGAGGACGTGAG |
| | | CCAGGCCTCACCAAGCGAGTCCGAAGCTCGGTTCAGAAT |
| | | CGACTCTGTCAGTGAAGGAAACGCCGGCCCTTACAGATG |
| | | CATCTACTATAAGCCCCCTAAATGGTCAGAGCAGAGCGAT |
| | | TATCTGGAACTGCTGGTGAAG |
| 29 | LAIR1ex + LS1R | EDLPRPSISAEPGTVIPLGSHVTFVCRGPVGVQTFRLERESRSLY |
| | aa | SDTEDVSQASPSESEARFRIDSVSEGNAGPYRCIYYKPRKWSEQ |
| | | SDYLELLVK |
| 30 | LAIR1ex + LS1R | GAGGACCTGCCCCGCCCTAGCATCTCCGCAGAACCAGG |
| | nucl | GACCGTGATTCCCCTGGGCTCCCACGTGACATTCGTCTGC |
| | | AGGGGCCCCGTGGGAGTCCAGACTTTTAGGCTGGAGCG |
| | | CGAATCTCGAAGTCTGTACTCCGACACCGAGGACGTGAG |
| | | CCAGGCCTCACCTAGCGAGTCCGAAGCTCGGTTCAGAAT |
| | | CGACTCTGTCAGTGAAGGAAACGCCGGCCCTTACAGATG |
| | | CATCTACTATAAGCCAAGAAAATGGTCAGAGCAGAGCGA |
| | | TTATCTGGAACTGCTGGTGAAG |
| 31 | LAIR1ex + LS1S2R | EDLPRPSISAEPGTVIPLGSHVTFVCRGPVGVQTFRLERESRSLY |
| | aa | SDTEDVSQASPSESEARFRIDSVSEGNAGPYRCIYYKSRKWSEQ |
| | | SDYLELLVK |
| 32 | LAIR1ex + LS1S2R | GAGGACCTGCCCCGCCCTAGCATCTCCGCAGAACCAGG |
| | nucl | GACCGTGATTCCCCTGGGCTCCCACGTGACATTCGTCTGC |
| | | AGGGGCCCCGTGGGAGTCCAGACTTTTAGGCTGGAGCG |
| | | CGAATCTCGAAGTCTGTACTCCGACACCGAGGACGTGAG |
| | | CCAGGCCTCACCTAGCGAGTCCGAAGCTCGGTTCAGAAT |
| | | CGACTCTGTCAGTGAAGGAAACGCCGGCCCATACAGATG |
| | | CATCTACTATAAGAGCAGAAAATGGTCAGAGCAGAGCGA |
| | | TTATCTGGAACTGCTGGTGAAG |
| 33 | LAIR1ex + LS1T aa | EDLPRPSISAEPGTVIPLGSHVTFVCRGPVGVQTFRLERESRSLY |
| | | SDTEDVSQTSPSESEARFRIDSVSEGNAGPYRCIYYKPPKWSEQ |
| | | SDYLELLVK |
| 34 | LAIR1ex + LS1T | GAGGACCTGCCAAGACCCAGCATCTCCGCCGAACCTGG |
| | nucl | GACTGTGATTCCACTGGGCTCCCACGTGACCTTCGTCTGC |
| | | AGAGGCCCCGTGGGAGTCCAGACCTTCCGGCTGGAGCG |
| | | CGAATCTCGAAGTCTGTACTCCGACACCGAGGACGTGAG |
| | | CCAGACATCACCCAGCGAGTCCGAAGCCCGGTTCAGAAT |
| | | CGACTCTGTCAGTGAAGGAAACGCTGGCCCTTACAGATG |
| | | CATCTACTATAAGCCCCCTAAATGGTCAGAGCAGAGCGAT |
| | | TATCTGGAACTGCTGGTGAAG |
| 35 | LAIR1ex + LS1TR | EDLPRPSISAEPGTVIPLGSHVTFVCRGPVGVQTFRLERESRSLY |
| | aa | SDTEDVSQTSPSESEARFRIDSVSEGNAGPYRCIYYKPRKWSEQ |
| | | SDYLELLVK |
| 36 | LAIR1ex + LS1TR | GAGGACCTGCCTAGACCTAGCATCTCCGCCGAACCAGGG |
| | nucl | ACTGTGATTCCCCTGGGCTCCCACGTGACCTTCGTCTGCA |
| | | GAGGCCCCGTGGGAGTCCAGACCTTCCGGCTGGAGCGC |
| | | GAATCTCGAAGTCTGTACTCCGACACCGAGGACGTGAGC |
| | | CAGACATCACCTAGCGAGTCCGAAGCCCGGTTCAGAATC |
| | | GACTCTGTCAGTGAAGGAAACGCTGGCCCTTACAGATGC |
| | | ATCTACTATAAGCCAAGAAAATGGTCAGAGCAGAGCGAT |
| | | TATCTGGAACTGCTGGTGAAG |
| 37 | LAIR1ex + LS1TS2 | EDLPRPSISAEPGTVIPLGSHVTFVCRGPVGVQTFRLERESRSLY |
| | R aa | SDTEDVSQTSPSESEARFRIDSVSEGNAGPYRCIYYKSRKWSEQ |
| | | SDYLELLVK |
| 38 | LAIR1ex + LS1TS2 | GAGGACCTGCCTAGACCTAGCATCTCCGCCGAACCAGGG |
| | R nucl | ACTGTGATTCCCCTGGGCTCCCACGTGACCTTCGTCTGCA |
| | | GAGGCCCCGTGGGAGTCCAGACCTTCCGGCTGGAGCGC |
| | | GAATCTCGAAGTCTGTACTCCGACACCGAGGACGTGAGC |
| | | CAGACATCACCTAGCGAGTCCGAAGCCCGGTTCAGAATC |
| | | GACTCTGTCAGTGAAGGAAACGCTGGCCCATACAGATGC |
| | | ATCTACTATAAGAGCAGAAAATGGTCAGAGCAGAGCGAT |
| | | TATCTGGAACTGCTGGTGAAG |
| 39 | LAIR1ex + LS2R | EDLPRPSISAEPGTVIPLGSHVTFVCRGPVGVQTFRLERESRSLY |
| | aa | NDTEDVSQASPSESEARFRIDSVSEGNAGPYRCIYYKSRKWSE |
| | | QSDYLELLVK |
| 40 | LAIR1ex + LS2R | GAGGACCTGCCCCGCCCTAGCATCTCCGCAGAACCAGG |
| | nucl | GACCGTGATTCCCCTGGGCTCCCACGTGACATTCGTCTGC |
| | | AGGGGCCCCGTGGGAGTCCAGACTTTTAGGCTGGAGCG |
| | | CGAATCTCGAAGTCTGTACAACGACACCGAGGACGTGAG |
| | | CCAGGCCTCACCTAGCGAGTCCGAAGCTCGGTTCAGAAT |
| | | CGACTCTGTCAGTGAAGGAAATGCCGGCCCATACAGATG |
| | | CATCTACTATAAGTCTAGAAAATGGTCAGAGCAGAGCGAT |
| | | TATCTGGAACTGCTGGTGAAG |
| 41 | LAIR1ex + LT aa | EDLPRPSISAEPGTVIPLGSHVTFVCRGPVGVQTFRLERESRSLY |
| | | NDTEDVSQTSPSESEARFRIDSVSEGNAGPYRCIYYKPPKWSE |
| | | QSDYLELLVK |
| 42 | LAIR1ex + LT nucl | GAGGACCTGCCAAGACCCAGCATCTCCGCCGAACCTGG |
| | | GACTGTGATTCCACTGGGCTCCCACGTGACCTTCGTCTGC |
| | | AGAGGCCCCGTGGGAGTCCAGACCTTCCGGCTGGAGCG |
| | | CGAATCTCGAAGTCTGTACAACGACACCGAGGACGTGAG |
| | | CCAGACATCACCCAGCGAGTCCGAAGCCCGGTTCAGAAT |
| | | CGACTCTGTCAGTGAAGGAAATGCTGGCCCTTACAGATG |
| | | CATCTACTATAAGCCCCCTAAATGGTCAGAGCAGAGCGAT |
| | | TATCTGGAACTGCTGGTGAAG |
| 43 | LAIR1ex + R aa | EDLPRPSISAEPGTVIPLGSHVTFVCRGPVGVQTFRLERESRSTY |
| | | NDTEDVSQASPSESEARFRIDSVSEGNAGPYRCIYYKPRKWSE |
| | | QSDYLELLVK |
| 44 | LAIR1ex + R nucl | GAGGACCTGCCCCGCCCTAGCATCTCCGCAGAACCAGG |
| | | GACTGTGATTCCCCTGGGCTCCCACGTGACCTTCGTCTGC |
| | | AGAGGCCCCGTGGGAGTCCAGACCTTCCGGCTGGAGCG |
| | | CGAATCTCGAAGTACCTACAACGACACAGAGGACGTGAG |
| | | CCAGGCCTCACCTAGCGAGTCCGAAGCTCGGTTCAGAAT |
| | | CGACTCTGTCAGTGAAGGAAATGCCGGCCCTTACAGATG |
| | | CATCTACTATAAGCCAAGAAAATGGTCAGAGCAGAGCGA |
| | | TTATCTGGAACTGCTGGTGAAG |
| 45 | LAIR1ex + S1 aa | EDLPRPSISAEPGTVIPLGSHVTFVCRGPVGVQTFRLERESRSTY |
| | | SDTEDVSQASPSESEARFRIDSVSEGNAGPYRCIYYKPPKWSEQ |
| | | SDYLELLVK |
| 46 | LAIR1ex + S1 nucl | GAGGACCTGCCAAGACCCAGCATCTCCGCAGAACCTGG |
| | | GACTGTGATTCCACTGGGCTCCCACGTGACCTTCGTCTGC |
| | | AGAGGCCCCGTGGGAGTCCAGACCTTCCGGCTGGAGCG |
| | | CGAATCTCGAAGTACCTACTCCGACACAGAGGACGTGAG |
| | | CCAGGCCTCACCCAGCGAGTCCGAAGCTCGGTTCAGAAT |
| | | CGACTCTGTCAGTGAAGGAAACGCCGGCCCTTACAGATG |
| | | CATCTACTATAAGCCCCCTAAATGGTCAGAGCAGAGCGAT |
| | | TATCTGGAACTGCTGGTGAAG |
| 47 | LAIR1ex + S1R aa | EDLPRPSISAEPGTVIPLGSHVTFVCRGPVGVQTERLERESRSTY |
| | | SDTEDVSQASPSESEARFRIDSVSEGNAGPYRCIYYKPRKWSEQ |
| | | SDYLELLVK |
| 48 | LAIR1ex + S1R | GAGGACCTGCCCCGCCCTAGCATCTCCGCAGAACCAGG |
| | nucl | GACTGTGATTCCCCTGGGCTCCCACGTGACCTTCGTCTGC |
| | | AGAGGCCCCGTGGGAGTCCAGACCTTCCGGCTGGAGCG |
| | | CGAATCTCGAAGTACCTACTCCGACACAGAGGACGTGAG |
| | | CCAGGCCTCACCTAGCGAGTCCGAAGCTCGGTTCAGAAT |
| | | CGACTCTGTCAGTGAAGGAAACGCCGGCCCTTACAGATG |
| | | CATCTACTATAAGCCAAGAAAATGGTCAGAGCAGAGCGA |
| | | TTATCTGGAACTGCTGGTGAAG |
| 49 | LAIR1ex + S1S2R | EDLPRPSISAEPGTVIPLGSHVTFVCRGPVGVQTFRLERESRSTY |
| | aa | SDTEDVSQASPSESEARFRIDSVSEGNAGPYRCIYYKSRKWSEQ |
| | | SDYLELLVK |
| 50 | LAIR1ex + S1S2R | GAGGACCTGCCCCGCCCTAGCATCTCCGCAGAACCAGG |
| | nucl | GACTGTGATTCCCCTGGGCTCCCACGTGACCTTCGTCTGC |
| | | AGAGGCCCCGTGGGAGTCCAGACCTTCCGGCTGGAGCG |
| | | CGAATCTCGAAGTACCTACTCCGACACAGAGGACGTGAG |
| | | CCAGGCCTCACCTAGCGAGTCCGAAGCTCGGTTCAGAAT |
| | | CGACTCTGTCAGTGAAGGAAACGCCGGCCCATACAGATG |
| | | CATCTACTATAAGAGCAGAAAATGGTCAGAGCAGAGCGA |
| | | TTATCTGGAACTGCTGGTGAAG |
| 51 | LAIR1ex + S1T aa | EDLPRPSISAEPGTVIPLGSHVTFVCRGPVGVQTFRLERESRSTY |
| | | SDTEDVSQTSPSESEARFRIDSVSEGNAGPYRCIYYKPPKWSEQ |
| | | SDYLELLVK |
| 52 | LAIR1ex + S1T | GAGGACCTGCCAAGACCCAGCATCTCCGCCGAACCTGG |
| | nucl | GACTGTGATTCCACTGGGCTCCCACGTGACCTTCGTCTGC |
| | | AGAGGCCCCGTGGGAGTCCAGACCTTCCGGCTGGAGCG |
| | | CGAATCTCGAAGTACCTACTCCGACACAGAGGACGTGAG |
| | | CCAGACCTCACCCAGCGAGTCCGAAGCCCGGTTCAGAAT |
| | | CGACTCTGTCAGTGAAGGAAACGCTGGCCCTTACAGATG |
| | | CATCTACTATAAGCCCCCTAAATGGTCAGAGCAGAGCGAT |
| | | TATCTGGAACTGCTGGTGAAG |
| 53 | LAIR1ex + S2 aa | EDLPRPSISAEPGTVIPLGSHVTFVCRGPVGVQTFRLERESRSTY |
| | | NDTEDVSQASPSESEARFRIDSVSEGNAGPYRCIYYKSPKWSE |
| | | QSDYLELLVK |
| 54 | LAIR1ex + S2 nucl | GAGGACCTGCCCAGACCTAGCATCTCCGCAGAACCAGG |
| | | GACTGTGATTCCCCTGGGCTCCCACGTGACCTTCGTCTGC |
| | | AGAGGCCCCGTGGGAGTCCAGACCTTCCGGCTGGAGCG |
| | | CGAATCTCGAAGTACCTACAACGACACAGAGGACGTGAG |
| | | CCAGGCCTCACCTAGCGAGTCCGAAGCTCGGTTCAGAAT |
| | | CGACTCTGTCAGTGAAGGAAATGCCGGCCCTTACAGATG |
| | | CATCTACTATAAGTCTCCAAAATGGTCAGAGCAGAGCGAT |
| | | TATCTGGAACTGCTGGTGAAG |
| 55 | LAIR1ex + S2R aa | EDLPRPSISAEPGTVIPLGSHVTFVCRGPVGVQTFRLERESRSTY |
| | | NDTEDVSQASPSESEARFRIDSVSEGNAGPYRCIYYKSRKWSE |
| | | QSDYLELLVK |
| 56 | LAIR1ex + S2R | GAGGACCTGCCCCGCCCTAGCATCTCCGCAGAACCAGG |
| | nucl | GACTGTGATTCCCCTGGGCTCCCACGTGACCTTCGTCTGC |
| | | AGAGGCCCCGTGGGAGTCCAGACCTTCCGGCTGGAGCG |
| | | CGAATCTCGAAGTACCTACAACGACACAGAGGACGTGAG |
| | | CCAGGCCTCACCTAGCGAGTCCGAAGCTCGGTTCAGAAT |
| | | CGACTCTGTCAGTGAAGGAAATGCCGGCCCATACAGATG |
| | | CATCTACTATAAGTCTAGAAAATGGTCAGAGCAGAGCGAT |
| | | TATCTGGAACTGCTGGTGAAG |
| 57 | LAIR1ex + T aa | EDLPRPSISAEPGTVIPLGSHVTFVCRGPVGVQTFRLERESRSTY |
| | | NDTEDVSQTSPSESEARFRIDSVSEGNAGPYRCIYYKPPKWSE |
| | | QSDYLELLVK |
| 58 | LAIR1ex + T nucl | GAGGACCTGCCAAGACCCAGCATCTCCGCCGAACCTGG |
| | | GACTGTGATTCCACTGGGCTCCCACGTGACCTTCGTCTGC |
| | | AGAGGCCCCGTGGGAGTCCAGACCTTCCGGCTGGAGCG |
| | | CGAATCTCGAAGTACCTACAACGACACAGAGGACGTGAG |
| | | CCAGACCTCACCCAGCGAGTCCGAAGCCCGGTTCAGAAT |
| | | CGACTCTGTCAGTGAAGGAAATGCTGGCCCTTACAGATG |
| | | CATCTACTATAAGCCCCCTAAATGGTCAGAGCAGAGCGAT |
| | | TATCTGGAACTGCTGGTGAAG |
| 59 | LAIR1ex + TS2R | EDLPRPSISAEPGTVIPLGSHVTFVCRGPVGVQTFRLERESRSTY |
| | aa | NDTEDVSQTSPSESEARFRIDSVSEGNAGPYRCIYYKSRKWSE |
| | | QSDYLELLVK |
| 60 | LAIR1ex + TS2R | GAGGACCTGCCTAGACCTAGCATCTCCGCCGAACCAGGG |
| | nucl | ACTGTGATTCCCCTGGGCTCCCACGTGACCTTCGTCTGCA |
| | | GAGGCCCCGTGGGAGTCCAGACCTTCCGGCTGGAGCGC |
| | | GAATCTCGAAGTACCTACAACGACACAGAGGACGTGAGC |
| | | CAGACCTCACCTAGCGAGTCCGAAGCCCGGTTCAGAATC |
| | | GACTCTGTCAGTGAAGGAAATGCTGGCCCATACAGATGC |
| | | ATCTACTATAAGTCTAGAAAATGGTCAGAGCAGAGCGATT |
| | | ATCTGGAACTGCTGGTGAAG |

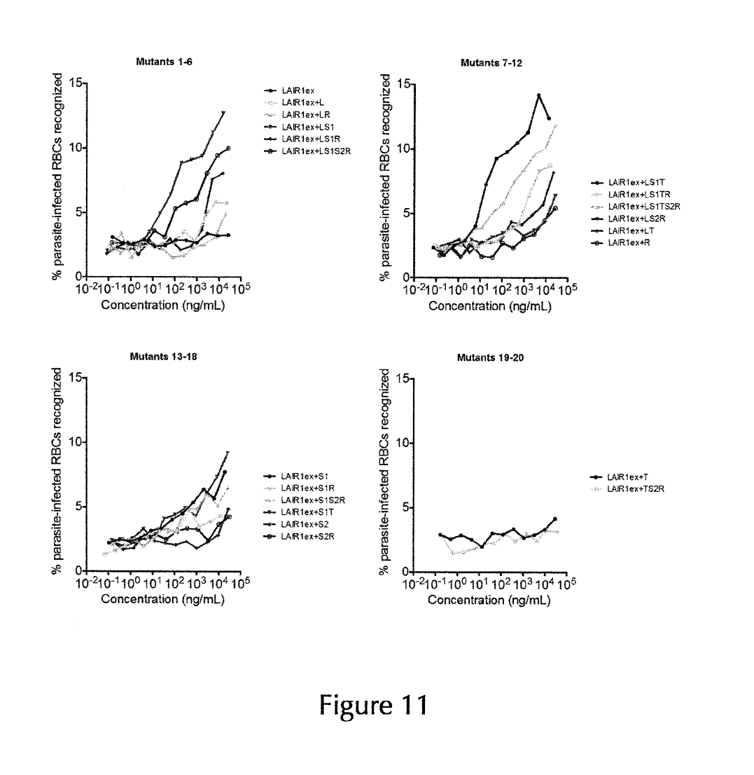

The 20 fusion proteins were expressed in HEK293 cells and the binding to P. falciparum was assessed by staining IEs, as described in Example 1. The results are shown in FIG. 11. These results show that native human LAIR-1 (“LAIR1ex”) does not bind to infected IEs and that at least one of the mutations T67L (“L”); N69S (“S1”); A77T (“T”); P106S (“S2”) and P107R (“R”) is necessary for gaining binding to IE.

### Example 10

### Influence of the Mutations of LAIR-1 Fragment on Binding to Collagen

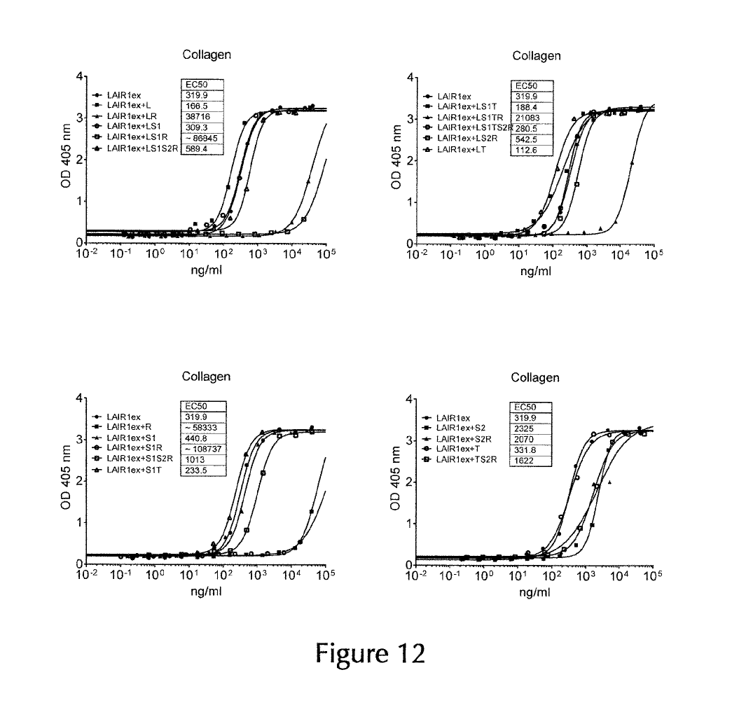

Native human LAIR-1 is well-known to bind collagen, in particular via its extracellular domain (T. Harma C. Brondijk, Talitha de Ruiter, Joost Ballering, Hans Wienk, Robert Jan Lebbink, Hugo van Ingen, Rolf Boelens, Richard W. Farndale, Linde Meyaard, and Eric G. Huizinga (2010): Crystal structure and collagen-binding site of immune inhibitory receptor LAIR-1: unexpected implications for collagen binding by platelet receptor GPVI. Blood 115:7). To identify whether the five mutations influence binding to collagen, the 20 fusion proteins of Example 9 were expressed in HEK293 cells and the binding to collagen was assessed by ELISA. Briefly ELISA plates were coated with Collagen type 1, blocked with PBS 1% BSA, followed by incubation with supernatants and a secondary anti-human (Fc-specific) antibody for detection. The results are shown in FIG. 12. These results show that in particular mutation P107R appears to deteriorate binding to collagen (FIG. 12).

### Example 11

### Identification of the P. falciparum Antigen(s) Recognized by MGD21

To identify the antigen(s) recognized by the LAIR1-containing antibodies, stable P. falciparum 3D7 lines, which were enriched (3D7-MGD21′) or depleted (3D7-MGD21⁻) of MGD21 reactivity were generated.

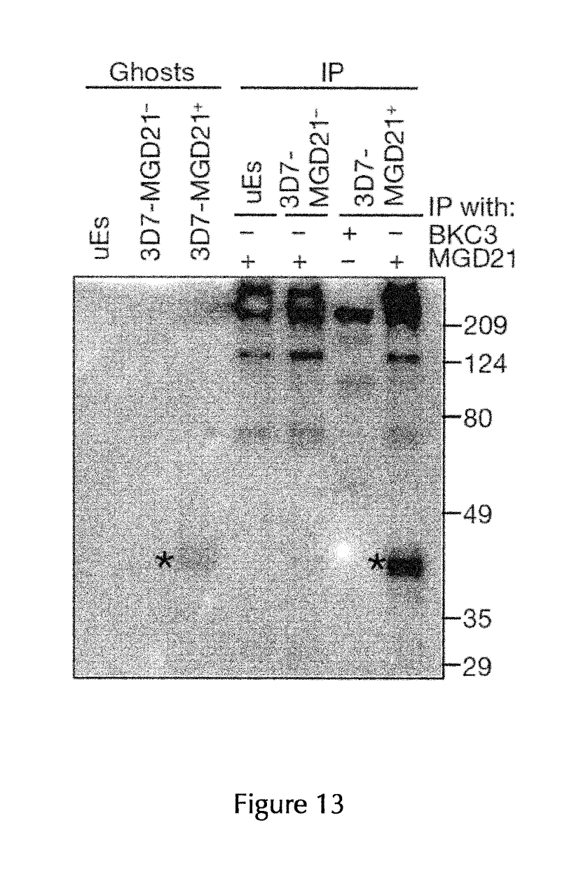

To investigate MGD21 binding to erythrocyte ghosts and MGD21 immunoprecipitates (IP) prepared from 3D7-MGD21⁺ and 3D7-MGD21⁻ IEs, a western blot was performed. Controls included uninfected erythrocytes (uEs) and immunoprecipitates with an irrelevant antibody (BKC3). Anti-human IgG was used as the secondary antibody, resulting in detection of antibodies used for immunoprecipitation alongside antigens of interest. As shown in FIG. 13, western blot analysis revealed two specific MGD21-reactive bands of 40-45 kilodaltons (kDa) in erythrocyte ghosts and in MGD21 immunoprecipitates prepared from 3D7-MGD21 IEs.

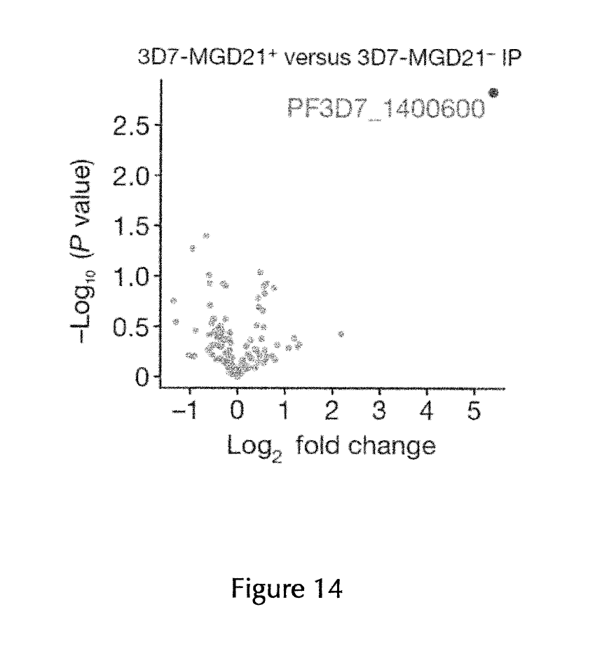

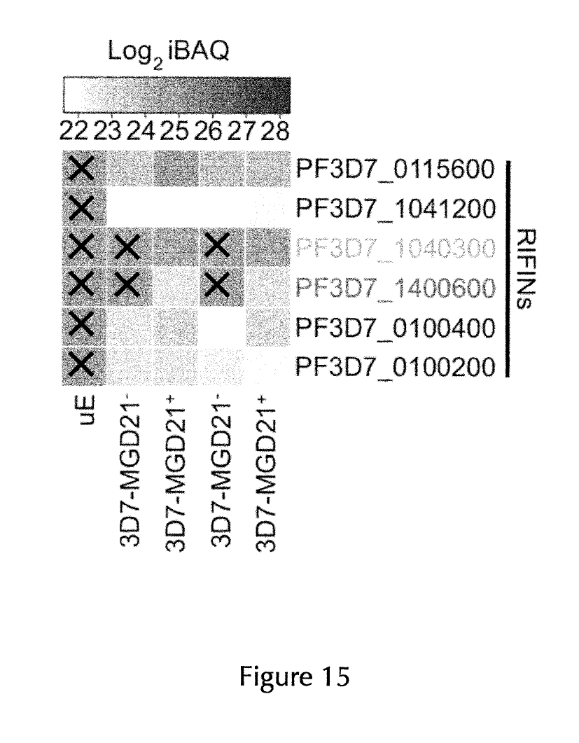

Next, analysis of the MGD21 immunoprecipitates by liquid chromatography coupled with mass spectrometry (LC-MS) was performed. As shown in FIG. 14, this experiment revealed that a member of the A-type RIFIN family (PF3D7_1400600 was significantly enriched in 3D7-MGD21⁻ immunoprecipitates as compared to 3D7-MGD21⁻ immunoprecipitates (log₂ fold change >2; P<0.01). Moreover, RIFIN expression levels in erythrocyte ghosts prepared from 3D7-MGD21⁺ and 3D7-MGD21⁻ IEs revealed that PF3D7_1400600 and a second A-type RIFIN (PF3D7_1040300) were also present in 3D7-MGD21⁺ but not in 3D7-MGD21⁻ ghosts in the absence of immunoprecipitation (FIG. 15). In contrast, four other RIFINs, including one recently characterized for its capacity to induce rosetting (PF3D7_0100400), were detected in similar amounts in both 3D7-MGD21⁺ and 3D7-MGD21⁻ ghosts (FIG. 15).

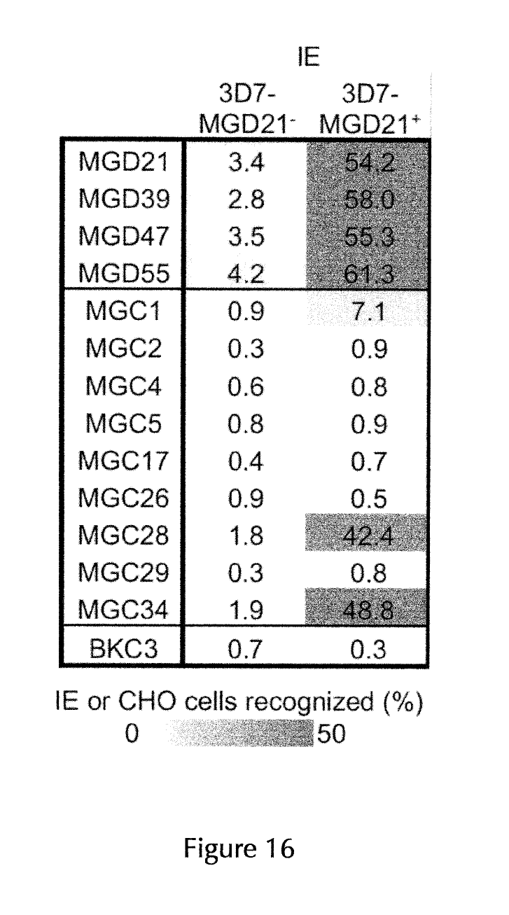

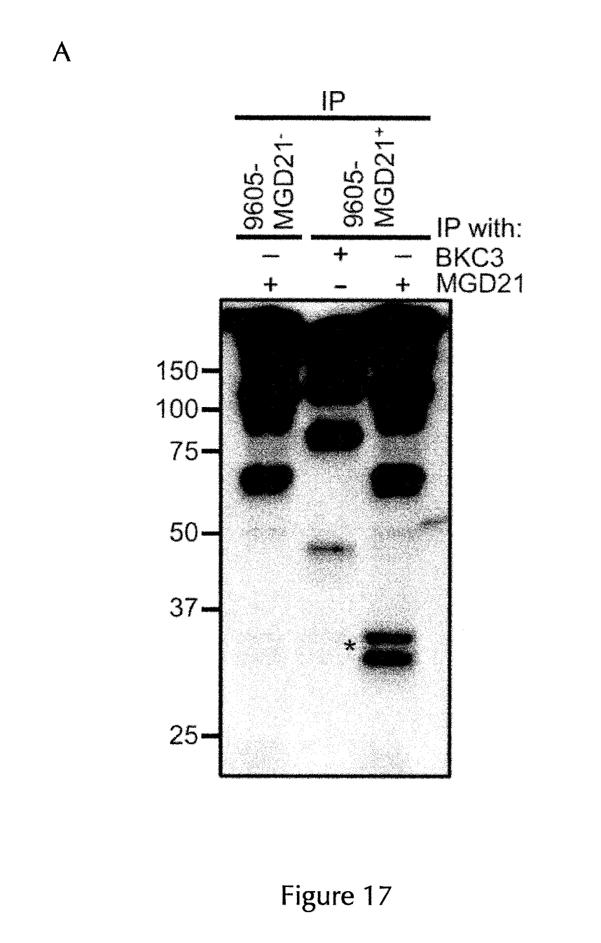

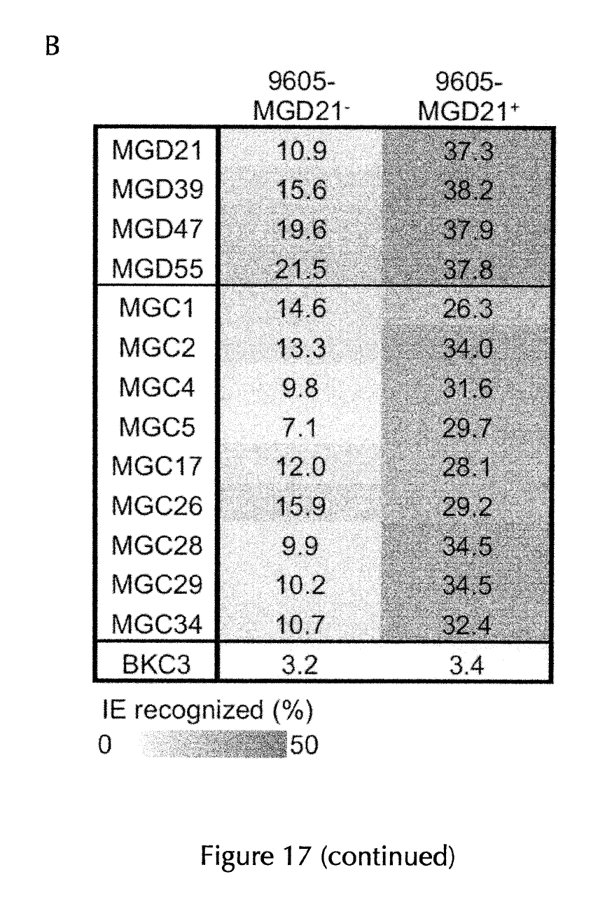

In the next step, recognition of 3D7-MGD21⁺ IEs and 3D7-MGD21⁻ IEs by other broadly reactive antibodies from donors C (MGC1, MGC2, MGC4, MGCS, MGC17, MGC26, MGC28, MGC29, MGC34) and D (MGD21, MGD39, MGD47, MGD55) were investigated. BKC3 was used as negative control antibody. As shown in FIG. 16, this experiment revealed that enrichment for 3D7-MGD21⁺ IEs greatly increased recognition by all the other broadly reactive antibodies from donor D tested and, notably, by two broadly reactive antibodies from donor C. These results suggest that these antibodies recognize the same antigens. Similar results were also obtained with the Kenyan isolate 9605 (FIG. 17A-B).

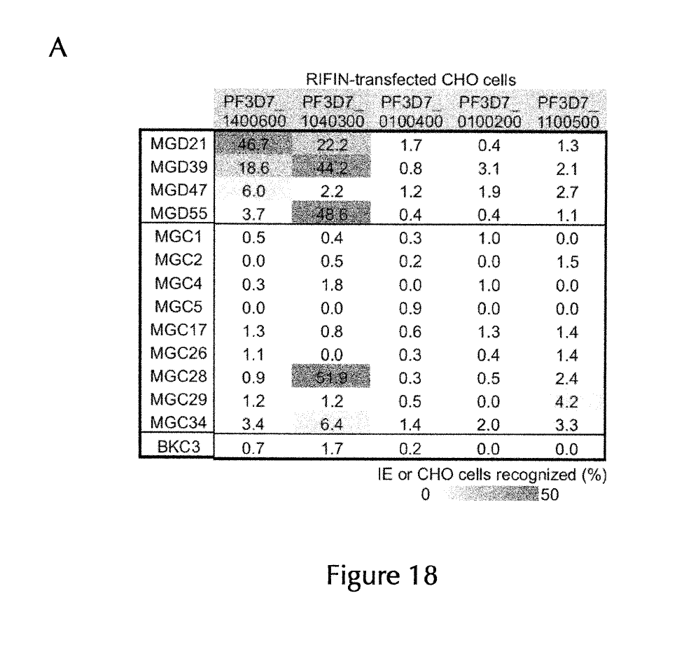

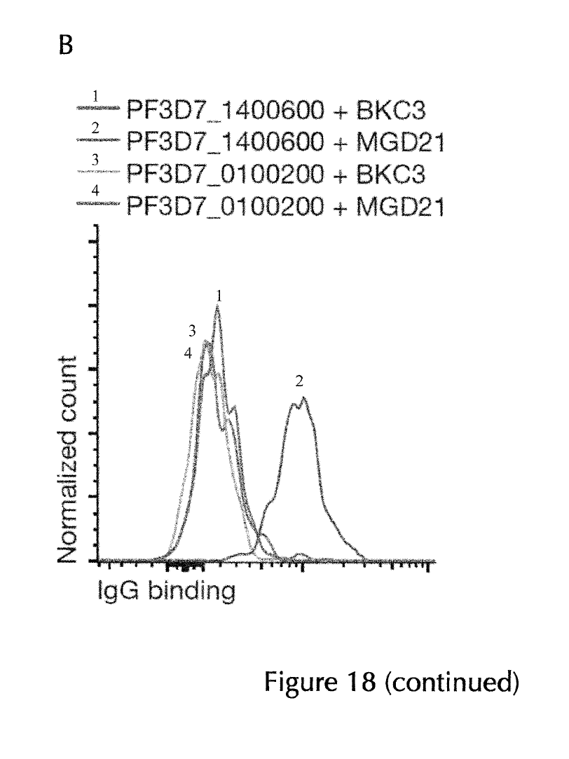

The binding of the LAIR1-containing antibodies to specific RIFINs was determined by use of CHO cells transfected with PF3D7_1400600 and PF3D7_1040300, PF3D7_0100400, PF3D7_0100200 and PF3D7_1100500. As shown in FIG. 18A, this experiment confirmed the finding that MGD21 stained CHO cells transfected with the candidate antigens PF3D7_1400600 and PF3D7_1040300, but not with irrelevant RIFINs that were similarly expressed (PF3D7_0100400 and PF3D7_0100200) or not detected (PF3D7_1100500) in 3D7-MGD21⁺ and 3D7-MGD21⁻ ghosts. FIG. 18B shows MGD21 and BKC3 staining of CHO cells transfected with a specific (PF3D7_1400600) or an irrelevant (PF3D7_0100200) RIFIN, confirming that the specificity of the binding of MGD21 to the specific RIFIN PF3D7_1400600.

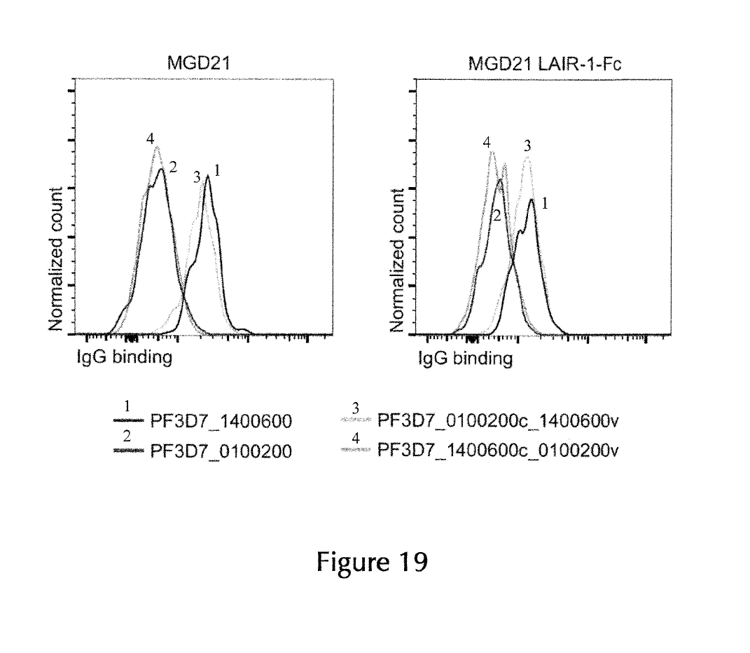

Furthermore, CHO cells were transfected with a specific (PF3D7_1400600) or an irrelevant (PF3D7_0100200) RIFIN as well as with a RIFIN chimaera containing the constant region of PF3D7_0100200 and the variable region of PF3D7_1400600 and a RIFIN chimaera containing the constant region of PF3D7_1400600 and the variable region of PF3D7_0100200. MGD21 and an Fc fusion protein containing the MGD21 LAIR1 domain stained only those CHO cells, which were transfected with the specific RIFIN PF3D7_1400600 or with the RIFIN chimaera containing the constant region of PF3D7_0100200 and the variable region of PF3D7_1400600, but not cells transfected with the inverse chimaera. Results are shown in FIG. 19, indicating that MGD21 binds to the variable region.

Collectively, the results obtained in Example 11 indicate that the LAIR1-containing antibodies recognize specific members of the RIFIN family in different P. falciparum isolates.

In particular, these results identify RIFIN PF3D7_1400600 (amino acid sequence according to SEQ ID NO: 105, nucleotide sequence according to SEQ ID NO: 106) as one major target of the mutated LAIR-1 fragment in P. falciparum and RIFIN PF3D7_1040300 (amino acid sequence according to SEQ ID NO: 538, nucleotide sequence according to SEQ ID NO: 539) as another target of the mutated LAIR-1 fragment in P. falciparum.

Since RIFINs are highly polymorphic in different strains and the mutated LAIR-1 fragment according to the present invention binds to erythrocytes infected by different P. falciparum strains, it is anticipated that the mutated LAIR-1 fragment according to the present invention will recognize additional RIFINs.

1.-74. (canceled)

75. A protein comprising at least amino acids 67 to 107 of native human LAIR-1, wherein said LAIR-1 fragment comprises:
i) 1, 2, 3, 4, or 5 mutations in comparison to native human LAIR-1 at one or more of the following five positions: T67, N69, A77, P106, and P107; and
ii) optionally, one or more further mutations at a position different from T67, N69, A77, P106, and P107 in comparison to native human LAIR-1,
wherein said LAIR-1 fragment has at least 70% amino acid sequence identity to amino acids 67 to 107 of native human LAIR-1 (SEQ ID NO: 9).

76. The protein according to claim 75 comprising at least the mutation N69S and/or T67L.

77. The protein according to claim 75, wherein said LAIR-1 fragment includes a mutation in at least two of the following five positions: T67, N69, A77, P106, and P107.

78. The protein according to claim 75, wherein said LAIR-1 fragment includes a mutation at each of the following five positions: T67, N69, A77, P106, and P107.

79. The protein according to claim 77 or 78, wherein the threonine residue at position T67 is substituted by an amino acid selected from the group consisting of leucine, glycine, isoleucine, arginine and lysine.

80. The protein according to claim 77 or 78, wherein the asparagine residue at position N69 is substituted by an amino acid selected from the group consisting of serine and threonine.

81. The protein according to claim 77 or 78, wherein the alanine residue at position A77 is substituted by an amino acid selected from the group consisting of threonine, proline and valine.

82. The protein according to claim 77 or 78, wherein the proline residue at position P106 is substituted by an amino acid selected from the group consisting of serine, alanine and aspartic acid.

83. The protein according to claim 77 or 78, wherein the proline residue at position P107 is substituted by an amino acid selected from the group consisting of serine and arginine.

84. The protein according to claim 75 comprising the LAIR-1 fragment according to SEQ ID NO: 19, 20, 21 or 22.

85. The protein according to claim 75, wherein the LAIR-1 fragment comprises at least the following mutations in comparison to native human LAIR-1: (i) T67L and A77V or A77T, and/or (ii) T67L, N69S, A77T, P106S, and P107R.

86. The protein according to claim 75, wherein the protein binds to a Plasmodium falciparum surface antigen.

87. The protein according to claim 75, wherein the protein neutralizes infection by Plasmodium falciparum.

88. The protein according to claim 75, wherein the protein does not bind collagen.

89. The protein according to claim 75 comprising a LAIR-1 fragment having an amino acid sequence according to any of SEQ ID NOs: 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101 and 103 or according to a functional sequence variant thereof.

90. The protein according to claim 75, wherein the protein is an antibody.

91. The protein according to claim 90, wherein the protein comprises a heavy chain comprising the amino acid sequence of (i) SEQ ID NO: 120 for CDRH1, SEQ ID NO: 121 for CDRH2 and SEQ ID NO: 122 for CDRH3 or functional sequence variants thereof; (ii) SEQ ID NO: 138 for CDRH1, SEQ ID NO: 139 for CDRH2 and SEQ ID NO: 140 for CDRH3 or functional sequence variants thereof; (iii) SEQ ID NO: 156 for CDRH1, SEQ ID NO: 157 for CDRH2 and SEQ ID NO: 158 for CDRH3 or functional sequence variants thereof; (iv) SEQ ID NO: 174 for CDRH1, SEQ ID NO: 175 for CDRH2 and SEQ ID NO: 176 for CDRH3 or functional sequence variants thereof; (v) SEQ ID NO: 192 for CDRH1, SEQ ID NO: 193 for CDRH2 and SEQ ID NO: 194 for CDRH3 or functional sequence variants thereof; (vi) SEQ ID NO: 210 for CDRH1, SEQ ID NO: 211 for CDRH2 and SEQ ID NO: 212 for CDRH3 or functional sequence variants thereof; (vii) SEQ ID NO: 228 for CDRH1, SEQ ID NO: 229 for CDRH2 and SEQ ID NO: 330 for CDRH3 or functional sequence variants thereof; (viii) SEQ ID NO: 246 for CDRH1, SEQ ID NO: 247 for CDRH2 and SEQ ID NO: 248 for CDRH3 or functional sequence variants thereof; (ix) SEQ ID NO: 264 for CDRH1, SEQ ID NO: 265 for CDRH2 and SEQ ID NO: 266 for CDRH3 or functional sequence variants thereof; (x) SEQ ID NO: 282 for CDRH1, SEQ ID NO: 283 for CDRH2 and SEQ ID NO: 284 for CDRH3 or functional sequence variants thereof; (xi) SEQ ID NO: 300 for CDRH1, SEQ ID NO: 301 for CDRH2 and SEQ ID NO: 302 for CDRH3 or functional sequence variants thereof; (xii) SEQ ID NO: 318 for CDRH1, SEQ ID NO: 319 for CDRH2 and SEQ ID NO: 320 for CDRH3 or functional sequence variants thereof; (xiii) SEQ ID NO: 336 for CDRH1, SEQ ID NO: 337 for CDRH2 and SEQ ID NO: 338 for CDRH3 or functional sequence variants thereof; (xiv) SEQ ID NO: 354 for CDRH1, SEQ ID NO: 355 for CDRH2 and SEQ ID NO: 356 for CDRH3 or functional sequence variants thereof; (xv) SEQ ID NO: 372 for CDRH1, SEQ ID NO: 373 for CDRH2 and SEQ ID NO: 374 for CDRH3 or functional sequence variants thereof; (xvi) SEQ ID NO: 390 for CDRH1, SEQ ID NO: 391 for CDRH2 and SEQ ID NO: 392 for CDRH3 or functional sequence variants thereof; (xvii) SEQ ID NO: 408 for CDRH1, SEQ ID NO: 409 for CDRH2 and SEQ ID NO: 410 for CDRH3 or functional sequence variants thereof; (xviii) SEQ ID NO: 426 for CDRH1, SEQ ID NO: 427 for CDRH2 and SEQ ID NO: 428 for CDRH3 or functional sequence variants thereof; (xix) SEQ ID NO: 444 for CDRH1, SEQ ID NO: 445 for CDRH2 and SEQ ID NO: 446 for CDRH3 or functional sequence variants thereof; (xx) SEQ ID NO: 462 for CDRH1, SEQ ID NO: 463 for CDRH2 and SEQ ID NO: 464 for CDRH3 or functional sequence variants thereof; (xxi) SEQ ID NO: 480 for CDRH1, SEQ ID NO: 481 for CDRH2 and SEQ ID NO: 482 for CDRH3 or functional sequence variants thereof; (xxii) SEQ ID NO: 498 for CDRH1, SEQ ID NO: 499 for CDRH2 and SEQ ID NO: 500 for CDRH3 or functional sequence variants thereof; (xxiii) SEQ ID NO: 516 for CDRH1, SEQ ID NO: 517 for CDRH2 and SEQ ID NO: 518 for CDRH3 or functional sequence variants thereof; (xxiv) SEQ ID NO: 534 for CDRH1, SEQ ID NO: 535 for CDRH2 and SEQ ID NO: 536 for CDRH3 or functional sequence variants thereof; (xxv) SEQ ID NO: 552 for CDRH1, SEQ ID NO: 553 for CDRH2 and SEQ ID NO: 554 for CDRH3 or functional sequence variants thereof; or (xxvi) SEQ ID NO: 570 for CDRH1, SEQ ID NO: 571 for CDRH2 and SEQ ID NO: 572 for CDRH3 or functional sequence variants thereof.

92. The protein according to claim 90, wherein the protein comprises (i) a heavy chain variable region comprising the amino acid sequence of SEQ ID NO: 134 or a functional sequence variant thereof and a light chain variable region comprising the amino acid sequence of SEQ ID NO: 135 or a functional sequence variant thereof; or (ii) a heavy chain variable region comprising the amino acid sequence of SEQ ID NO: 152 or a functional sequence variant thereof and a light chain variable region comprising the amino acid sequence of SEQ ID NO: 153 or a functional sequence variant thereof; or (iii) a heavy chain variable region comprising the amino acid sequence of SEQ ID NO: 170 or a functional sequence variant thereof and a light chain variable region comprising the amino acid sequence of SEQ ID NO: 171 or a functional sequence variant thereof; or (iv) a heavy chain variable region comprising the amino acid sequence of SEQ ID NO: 188 or a functional sequence variant thereof and a light chain variable region comprising the amino acid sequence of SEQ ID NO: 189 or a functional sequence variant thereof; or (v) a heavy chain variable region comprising the amino acid sequence of SEQ ID NO: 206 or a functional sequence variant thereof and a light chain variable region comprising the amino acid sequence of SEQ ID NO: 207 or a functional sequence variant thereof; or (vi) a heavy chain variable region comprising the amino acid sequence of SEQ ID NO: 224 or a functional sequence variant thereof and a light chain variable region comprising the amino acid sequence of SEQ ID NO: 225 or a functional sequence variant thereof; or (vii) a heavy chain variable region comprising the amino acid sequence of SEQ ID NO: 242 or a functional sequence variant thereof and a light chain variable region comprising the amino acid sequence of SEQ ID NO: 243 or a functional sequence variant thereof; or (viii) a heavy chain variable region comprising the amino acid sequence of SEQ ID NO: 260 or a functional sequence variant thereof and a light chain variable region comprising the amino acid sequence of SEQ ID NO: 261 or a functional sequence variant thereof; or (ix) a heavy chain variable region comprising the amino acid sequence of SEQ ID NO: 278 or a functional sequence variant thereof and a light chain variable region comprising the amino acid sequence of SEQ ID NO: 279 or a functional sequence variant thereof; or (x) a heavy chain variable region comprising the amino acid sequence of SEQ ID NO: 296 or a functional sequence variant thereof and a light chain variable region comprising the amino acid sequence of SEQ ID NO: 297 or a functional sequence variant thereof; or (xi) a heavy chain variable region comprising the amino acid sequence of SEQ ID NO: 314 or a functional sequence variant thereof and a light chain variable region comprising the amino acid sequence of SEQ ID NO: 315 or a functional sequence variant thereof; or (xii) a heavy chain variable region comprising the amino acid sequence of SEQ ID NO: 332 or a functional sequence variant thereof and a light chain variable region comprising the amino acid sequence of SEQ ID NO: 333 or a functional sequence variant thereof; or (xiii) a heavy chain variable region comprising the amino acid sequence of SEQ ID NO: 350 or a functional sequence variant thereof and a light chain variable region comprising the amino acid sequence of SEQ ID NO: 351 or a functional sequence variant thereof; or (xiv) a heavy chain variable region comprising the amino acid sequence of SEQ ID NO: 368 or a functional sequence variant thereof and a light chain variable region comprising the amino acid sequence of SEQ ID NO: 369 or a functional sequence variant thereof; or (xv) a heavy chain variable region comprising the amino acid sequence of SEQ ID NO: 386 or a functional sequence variant thereof and a light chain variable region comprising the amino acid sequence of SEQ ID NO: 387 or a functional sequence variant thereof; or (xvi) a heavy chain variable region comprising the amino acid sequence of SEQ ID NO: 404 or a functional sequence variant thereof and a light chain variable region comprising the amino acid sequence of SEQ ID NO: 405 or a functional sequence variant thereof; or (xvii) a heavy chain variable region comprising the amino acid sequence of SEQ ID NO: 422 or a functional sequence variant thereof and a light chain variable region comprising the amino acid sequence of SEQ ID NO: 423 or a functional sequence variant thereof; or (xviii) a heavy chain variable region comprising the amino acid sequence of SEQ ID NO: 440 or a functional sequence variant thereof and a light chain variable region comprising the amino acid sequence of SEQ ID NO: 441 or a functional sequence variant thereof; or (xix) a heavy chain variable region comprising the amino acid sequence of SEQ ID NO: 458 or a functional sequence variant thereof and a light chain variable region comprising the amino acid sequence of SEQ ID NO: 459 or a functional sequence variant thereof; or (xx) a heavy chain variable region comprising the amino acid sequence of SEQ ID NO: 476 or a functional sequence variant thereof and a light chain variable region comprising the amino acid sequence of SEQ ID NO: 477 or a functional sequence variant thereof; or (xxi) a heavy chain variable region comprising the amino acid sequence of SEQ ID NO: 494 or a functional sequence variant thereof and a light chain variable region comprising the amino acid sequence of SEQ ID NO: 495 or a functional sequence variant thereof; or (xxii) a heavy chain variable region comprising the amino acid sequence of SEQ ID NO: 512 or a functional sequence variant thereof and a light chain variable region comprising the amino acid sequence of SEQ ID NO: 513 or a functional sequence variant thereof; or (xxiii) a heavy chain variable region comprising the amino acid sequence of SEQ ID NO: 530 or a functional sequence variant thereof and a light chain variable region comprising the amino acid sequence of SEQ ID NO: 531 or a functional sequence variant thereof; or (xxiv) a heavy chain variable region comprising the amino acid sequence of SEQ ID NO: 548 or a functional sequence variant thereof and a light chain variable region comprising the amino acid sequence of SEQ ID NO: 549 or a functional sequence variant thereof; or (xxv) a heavy chain variable region comprising the amino acid sequence of SEQ ID NO: 566 or a functional sequence variant thereof and a light chain variable region comprising the amino acid sequence of SEQ ID NO: 567 or a functional sequence variant thereof; or (xxvi) a heavy chain variable region comprising the amino acid sequence of SEQ ID NO: 584 or a functional sequence variant thereof and a light chain variable region comprising the amino acid sequence of SEQ ID NO: 585 or a functional sequence variant thereof.

93. A nucleic acid molecule comprising a polynucleotide encoding a protein according to claim 75.

94. A vector comprising the nucleic acid molecule according to claim 93.

95. A cell expressing the protein according to claim 75 or comprising the vector according to claim 94.

96. A LAIR-1 fragment protein comprising at least amino acids 67 to 107 of native human LAIR-1, wherein said LAIR-1 fragment protein comprises at least one mutation in comparison to native human LAIR-1 (SEQ ID NO: 9), said at least one mutation enabling binding to an antigen, and wherein said LAIR-1 fragment shows at least 70% amino acid sequence identity to amino acids 67 to 107 of native human LAIR-1 (SEQ ID NO: 9).

97. The protein according to claim 96, wherein the protein is an antibody, preferably a recombinant antibody.

98. A method of preventing and/or treating a disorder and/or a disease selected from the group consisting of: infectious diseases, autoimmune diseases, inflammatory diseases and cancers in a subject, wherein the method comprises administering to a subject in need thereof the protein according to claim 96.

99. A pharmaceutical composition comprising the protein according to claim 75 and/or the protein according to claim 96, a nucleic acid encoding one or both of said proteins, a vector comprising said nucleic acid, or a cell expressing one or both of said proteins or comprising said vector.

100. A method of diagnosing malaria, comprising contacting a sample suspected of containing a malaria parasite, or portion thereof, with the protein according to claim 75; and detecting a complex of the protein according to claim 75 and the malaria parasite, or portion thereof.

101. A method of limiting a Plasmodium falciparum infection, or lowering the risk of Plasmodium falciparum infection, comprising administering to a subject in need thereof, a therapeutically effective amount of :
iii) a protein comprising at least amino acids 67 to 107 of native human LAIR-1, wherein said LAIR-1 fragment comprises:
(a) 1, 2, 3, 4, or 5 mutations in comparison to native human LAIR-1 at one or more of the following five positions: T67, N69, A77, P106, and P107; and
(b) optionally, one or more further mutations at a position different from T67, N69, A77, P106, and P107 in comparison to native human LAIR-1,
wherein said LAIR-1 fragment shows at least 70% amino acid sequence identity to amino acids 67 to 107 of native human LAIR-1 (SEQ ID NO: 9),
iv) a nucleic acid encoding said protein,
v) a vector comprising said nucleic acid,
vi) a cell expressing said protein or comprising said vector, or
vii) a pharmaceutical composition comprising said protein and/or the protein according to claim 96, a nucleic acid encoding said protein, a vector comprising said nucleic acid molecule, or a cell expressing said protein or comprising said vector.

102. A method of preventing and/or treating malaria in a subject, wherein the method comprises administering to a subject in need thereof:
viii) a protein comprising at least amino acids 67 to 107 of native human LAIR-1, wherein said LAIR-1 fragment comprises:
(a) 1, 2, 3, 4, or 5 mutations in comparison to native human LAIR-1 at one or more of the following five positions: T67, N69, A77, P106, and P107; and
(b) optionally, one or more further mutations at a position different from T67, N69, A77, P106, and P107 in comparison to native human LAIR-1, wherein said LAIR-1 fragment shows at least 70% amino acid sequence identity to amino acids 67 to 107 of native human LAIR-1 (SEQ ID NO: 9),
ix) a nucleic acid encoding said protein,
x) a vector comprising said nucleic acid,
xi) a cell expressing said protein or comprising said vector, or
xii) a pharmaceutical composition comprising said protein and/or the protein according to claim 96, a nucleic acid encoding said protein, a vector comprising said nucleic acid molecule, or a cell expressing said protein or comprising said vector.


SEQUENCE LISTING (642 sequences)

SEQ ID NO: 1 - protein, 287 aa, Homo sapiens
MSPHPTALLGLVLCLAQTIHTQEEDLPRPSISAEPGTVIPLGSHVTFVCRGPVGVQTFRLERESRSTYNDTEDVSQASPSESEARFRIDSVSEGNAGPYRCIYYKPPKWSEQSDYLELLVKETSGGPDSPDTEPGSSAGPTQRPSDNSHNEHAPASQGLKAEHLYILIGVSVVFLFCLLLLVLFCLHRQNQIKQGPPRSKDEEQKPQQRPDLAVDVLERTADKATVNGLPEKDRETDTSALAAGSSQEVTYAQLDHWALTQRTARAVSPQSTKPMAESITYAAVARH

SEQ ID NO: 2 - DNA, 864 bp, Homo sapiens
atgtctccccaccccaccgccctcctgggcctagtgctctgcctggcccagaccatccacacgcaggaggaagatctgcccagaccctccatctcggctgagccaggcaccgtgatccccctggggagccatgtgactttcgtgtgccggggcccggttggggttcaaacattccgcctggagagggagagtagatccacatacaatgatactgaagatgtgtctcaagctagtccatctgagtcagaggccagattccgcattgactcagtaagtgaaggaaatgccgggccttatcgctgcatctattataagccccctaaatggtctgagcagagtgactacctggagctgctggtgaaagaaacctctggaggcccggactccccggacacagagcccggctcctcagctggacccacgcagaggccgtcggacaacagtcacaatgagcatgcacctgcttcccaaggcctgaaagctgagcatctgtatattctcatcggggtctcagtggtcttcctcttctgtctcctcctcctggtcctcttctgcctccatcgccagaatcagataaagcaggggccccccagaagcaaggacgaggagcagaagccacagcagaggcctgacctggctgttgatgttctagagaggacagcagacaaggccacagtcaatggacttcctgagaaggacagagagacggacacctcggccctggctgcagggagttcccaggaggtgacgtatgctcagctggaccactgggccctcacacagaggacagcccgggctgtgtccccacagtccacaaagcccatggccgagtccatcacgtatgcagccgttgccagacactga

SEQ ID NO: 3 - protein, 270 aa, Homo sapiens
MSPHPTALLGLVLCLAQTIHTQEEDLPRPSISAEPGTVIPLGSHVTFVCRGPVGVQTFRLERESRSTYNDTEDVSQASPSESEARFRIDSVSEGNAGPYRCIYYKPPKWSEQSDYLELLVKGPTQRPSDNSHNEHAPASQGLKAEHLYILIGVSVVFLFCLLLLVLFCLHRQNQIKQGPPRSKDEEQKPQQRPDLAVDVLERTADKATVNGLPEKDRETDTSALAAGSSQEVTYAQLDHWALTQRTARAVSPQSTKPMAESITYAAVARH

SEQ ID NO: 4 - DNA, 813 bp, Homo sapiens
atgtctccccaccccaccgccctcctgggcctagtgctctgcctggcccagaccatccacacgcaggaggaagatctgcccagaccctccatctcggctgagccaggcaccgtgatccccctggggagccatgtgactttcgtgtgccggggcccggttggggttcaaacattccgcctggagagggagagtagatccacatacaatgatactgaagatgtgtctcaagctagtccatctgagtcagaggccagattccgcattgactcagtaagtgaaggaaatgccgggccttatcgctgcatctattataagccccctaaatggtctgagcagagtgactacctggagctgctggtgaaaggacccacgcagaggccgtcggacaacagtcacaatgagcatgcacctgcttcccaaggcctgaaagctgagcatctgtatattctcatcggggtctcagtggtcttcctcttctgtctcctcctcctggtcctcttctgcctccatcgccagaatcagataaagcaggggccccccagaagcaaggacgaggagcagaagccacagcagaggcctgacctggctgttgatgttctagagaggacagcagacaaggccacagtcaatggacttcctgagaaggacagagagacggacacctcggccctggctgcagggagttcccaggaggtgacgtatgctcagctggaccactgggccctcacacagaggacagcccgggctgtgtccccacagtccacaaagcccatggccgagtccatcacgtatgcagccgttgccagacactga

SEQ ID NO: 5 - protein, 269 aa, Homo sapiens
MSPHPTALLGLVLCLAQTIHTQEDLPRPSISAEPGTVIPLGSHVTFVCRGPVGVQTFRLERESRSTYNDTEDVSQASPSESEARFRIDSVSEGNAGPYRCIYYKPPKWSEQSDYLELLVKGPTQRPSDNSHNEHAPASQGLKAEHLYILIGVSVVFLFCLLLLVLFCLHRQNQIKQGPPRSKDEEQKPQQRPDLAVDVLERTADKATVNGLPEKDRETDTSALAAGSSQEVTYAQLDHWALTQRTARAVSPQSTKPMAESITYAAVARH

SEQ ID NO: 6 - DNA, 810 bp, Homo sapiens
atgtctccccaccccaccgccctcctgggcctagtgctctgcctggcccagaccatccacacgcaggaggatctgcccagaccctccatctcggctgagccaggcaccgtgatccccctggggagccatgtgactttcgtgtgccggggcccggttggggttcaaacattccgcctggagagggagagtagatccacatacaatgatactgaagatgtgtctcaagctagtccatctgagtcagaggccagattccgcattgactcagtaagtgaaggaaatgccgggccttatcgctgcatctattataagccccctaaatggtctgagcagagtgactacctggagctgctggtgaaaggacccacgcagaggccgtcggacaacagtcacaatgagcatgcacctgcttcccaaggcctgaaagctgagcatctgtatattctcatcggggtctcagtggtcttcctcttctgtctcctcctcctggtcctcttctgcctccatcgccagaatcagataaagcaggggccccccagaagcaaggacgaggagcagaagccacagcagaggcctgacctggctgttgatgttctagagaggacagcagacaaggccacagtcaatggacttcctgagaaggacagagagacggacacctcggccctggctgcagggagttcccaggaggtgacgtatgctcagctggaccactgggccctcacacagaggacagcccgggctgtgtccccacagtccacaaagcccatggccgagtccatcacgtatgcagccgttgccagacactga

SEQ ID NO: 7 - protein, 209 aa, Homo sapiens
MSPHPTALLGLVLCLAQTIHTQEEDLPRPSISAEPGTVIPLGSHVTFVCRGPVGVQTFRLERESRSTYNDTEDVSQASPSESEARFRIDSVSEGNAGPYRCIYYKPPKWSEQSDYLELLVKETSGGPDSPDTEPGSSAGPTQRPSDNSHNEHAPASQGLKAEHLYILIGVSVVFLFCLLLLVLFCLHRQNQIKQGPPRSKDEEQKPQQR

SEQ ID NO: 8 - DNA, 630 bp, Homo sapiens
atgtctccccaccccaccgccctcctgggcctagtgctctgcctggcccagaccatccacacgcaggaggaagatctgcccagaccctccatctcggctgagccaggcaccgtgatccccctggggagccatgtgactttcgtgtgccggggcccggttggggttcaaacattccgcctggagagggagagtagatccacatacaatgatactgaagatgtgtctcaagctagtccatctgagtcagaggccagattccgcattgactcagtaagtgaaggaaatgccgggccttatcgctgcatctattataagccccctaaatggtctgagcagagtgactacctggagctgctggtgaaagaaacctctggaggcccggactccccggacacagagcccggctcctcagctggacccacgcagaggccgtcggacaacagtcacaatgagcatgcacctgcttcccaaggcctgaaagctgagcatctgtatattctcatcggggtctcagtggtcttcctcttctgtctcctcctcctggtcctcttctgcctccatcgccagaatcagataaagcaggggccccccagaagcaaggacgaggagcagaagccacagcagaggtga

SEQ ID NO: 9 - protein, 41 aa, Artificial Sequence
  LAIR-1 fragment
TYNDTEDVSQASPSESEARFRIDSVSEGNAGPYRCIYYKPP

SEQ ID NO: 10 - protein, 41 aa, Artificial Sequence
  LAIR-1 fragment
  X (1)..(1): X is any amino acid or no amino acid; however, if X (Pos. 3) is N, X (Pos. 11) is A, X (Pos. 40) is P and X (Pos. 41) is P, then X (Pos. 1) is any amino acid except T or no amino acid
  X (3)..(3): X is any amino acid; however, if X (Pos. 1) is T, X (Pos. 11) is A, X (Pos.40) is P and X (Pos. 41) is P, then X (Pos. 3) is any amino acid except N
  X (4)..(5): X is any amino acid
  X (7)..(7): X is any amino acid
  X (9)..(10): X is any amino acid
  X (11)..(11): X is any amino acid; however, if X (Pos. 1) is T, X (Pos. 3) is N, X (Pos. 40)  is P and X (Pos. 41)  is P, then X (Pos. 11)  is any amino acid except A
  X (12)..(12): X is any amino acid
  X (14)..(15): X is any amino acid
  X (22)..(23): X is any amino acid
  X (26)..(27): X is any amino acid
  X (29)..(30): X is any amino acid
  X (32)..(33): X is any amino acid
  X (36)..(36): X is any amino acid
  X (39)..(39): X is any amino acid
  X (40)..(40): X is any amino acid; however, if X (Pos. 1) is T, X (Pos. 3) is N, X (Pos. 11) is A and X (Pos. 41) is P, then X (Pos. 40) is any amino acid except P
  X (41)..(41): X is any amino acid; however, if X (Pos. 1) is T, X (Pos. 3) is N, X (Pos. 11) is A and X (Pos. 40) is P, then X (Pos. 41) is any amino acid except P
XYXXXEXVXXXXPXXSEARFRXXSVXXGXXGXXRCXYYXXX

SEQ ID NO: 11 - protein, 61 aa, Artificial Sequence
  LAIR-1 fragment
RGPVGVQTFRLERESRSTYNDTEDVSQASPSESEARFRIDSVSEGNAGPYRCIYYKPPKWS

SEQ ID NO: 12 - protein, 76 aa, Artificial Sequence
  LAIR-1 fragment
PLGSHVTFVCRGPVGVQTFRLERESRSTYNDTEDVSQASPSESEARFRIDSVSEGNAGPYRCIYYKPPKWSEQSDY

SEQ ID NO: 13 - protein, 91 aa, Artificial Sequence
  LAIR-1 fragment
SISAEPGTVIPLGSHVTFVCRGPVGVQTFRLERESRSTYNDTEDVSQASPSESEARFRIDSVSEGNAGPYRCIYYKPPKWSEQSDYLELLV

SEQ ID NO: 14 - protein, 98 aa, Homo sapiens
EDLPRPSISAEPGTVIPLGSHVTFVCRGPVGVQTFRLERESRSTYNDTEDVSQASPSESEARFRIDSVSEGNAGPYRCIYYKPPKWSEQSDYLELLVK

SEQ ID NO: 15 - protein, 61 aa, Artificial Sequence
  LAIR-1 fragment
  X (4)..(4): X is any amino acid or no amino acid (deletion mutation)
  X (6)..(7): X is any amino acid or no amino acid (deletion mutation)
  X (12)..(17): X is any amino acid or no amino acid (deletion mutation)
  X (18)..(18): X is any amino acid or no amino acid; however, if X (Pos. 20) is N, X (Pos. 28) is A, X (Pos. 57) is P and X (Pos. 58) is P, then X (Pos. 18) is any amino acid except T or no amino acid
  X (20)..(20): X is any amino acid; however, if X (Pos. 18) is T, X (Pos. 28) is A, X (Pos. 57) is P and X (Pos. 58) is P, then X (Pos. 20) is any amino acid except N
  X (21)..(22): X is any amino acid or no amino acid (deletion mutation)
  X (24)..(24): X is any amino acid or no amino acid (deletion mutation)
  X (26)..(27): X is any amino acid or no amino acid (deletion mutation)
  X (28)..(28): X is any amino acid; however, if X (Pos. 18) is T, X (Pos. 20) is N, X (Pos. 57) is P and X (Pos. 58) is P, then X (Pos. 28) is any amino acid except A
  X (29)..(29): X is any amino acid or no amino acid (deletion mutation)
  X (31)..(32): X is any amino acid or no amino acid (deletion mutation)
  X (39)..(40): X is any amino acid or no amino acid (deletion mutation)
  X (43)..(44): X is any amino acid or no amino acid (deletion mutation)
  X (46)..(47): X is any amino acid or no amino acid (deletion mutation)
  X (49)..(50): X is any amino acid or no amino acid (deletion mutation)
  X (53)..(53): X is any amino acid or no amino acid (deletion mutation)
  X (56)..(56): X is any amino acid or no amino acid (deletion mutation)
  X (57)..(57): X is any amino acid; however, if X (Pos. 18) is T, X (Pos. 20) is N, X (Pos. 28) is A and X (Pos. 58) is P, then X (Pos. 57) is any amino acid except P
  X (58)..(58): X is any amino acid; however, if X (Pos. 18) is T, X (Pos. 20) is N, X (Pos. 28) is A and X (Pos. 57) is P, then X (Pos. 58) is any amino acid except P
  X (59)..(59): X is any amino acid or no amino acid (deletion mutation)
RGPXGXXTFRLXXXXXXXYXXXEXVXXXXPXXSEARFRXXSVXXGXXGXXRCXYYXXXXWS

SEQ ID NO: 16 - protein, 76 aa, Artificial Sequence
  LAIR-1 fragment
  X (1)..(1): X is any amino acid or no amino acid (deletion mutation)
  X (5)..(6): X is any amino acid or no amino acid (deletion mutation)
  X (8)..(8): X is any amino acid or no amino acid (deletion mutation)
  X (14)..(14): X is any amino acid or no amino acid (deletion mutation)
  X (16)..(17): X is any amino acid or no amino acid (deletion mutation)
  X (22)..(27): X is any amino acid or no amino acid (deletion mutation)
  X (28)..(28): X is any amino acid or no amino acid; however, if X (Pos. 30) is N, X (Pos. 38) is A, X (Pos. 67) is P and X (Pos. 68) is P, then X (Pos. 28) is any amino acid except T or no amino acid
  X (30)..(30): X is any amino acid; however, if X (Pos. 28) is T, X (Pos. 38) is A, X (Pos. 67) is P and X (Pos. 68)  is P, then X (Pos. 30) is any amino acid except N
  X (31)..(32): X is any amino acid or no amino acid (deletion mutation)
  X (34)..(34): X is any amino acid or no amino acid (deletion mutation)
  X (36)..(37): X is any amino acid or no amino acid (deletion mutation)
  X (38)..(38): X is any amino acid; however, if X (Pos. 28) is T, X (Pos. 30) is N, X (Pos. 67) is P and X (Pos. 68) is P, then X (Pos. 38) is any amino acid except A
  X (39)..(39): X is any amino acid or no amino acid (deletion mutation)
  X (41)..(42): X is any amino acid or no amino acid (deletion mutation)
  X (49)..(50): X is any amino acid or no amino acid (deletion mutation)
  X (53)..(54): X is any amino acid or no amino acid (deletion mutation)
  X (56)..(57): X is any amino acid or no amino acid (deletion mutation)
  X (59)..(60): X is any amino acid or no amino acid (deletion mutation)
  X (63)..(63): X is any amino acid or no amino acid (deletion mutation)
  X (66)..(66): X is any amino acid or no amino acid (deletion mutation)
  X (67)..(67): X is any amino acid; however, if X (Pos. 28) is T, X (Pos. 30) is N, X (Pos. 38) is A and X (Pos. 68) is P, then X (Pos. 67) is any amino acid except P
  X (68)..(68): X is any amino acid; however, if X (Pos. 28) is T, X (Pos. 30) is N, X (Pos. 38) is A and X (Pos. 67) is P, then X (Pos. 68) is any amino acid except P
  X (69)..(69): X is any amino acid or no amino acid (deletion mutation)
  X (72)..(73): X is any amino acid or no amino acid (deletion mutation)
  X (75)..(76): X is any amino acid or no amino acid (deletion mutation)
XLGSXXTXVCRGPXGXXTFRLXXXXXXXYXXXEXVXXXXPXXSEARFRXXSVXXGXXGXXRCXYYXXXXWSXXSXX

SEQ ID NO: 17 - protein, 91 aa, Artificial Sequence
  LAIR-1 fragment
  X (1)..(2): X is any amino acid or no amino acid (deletion mutation)
  X (4)..(11): X is any amino acid or no amino acid (deletion mutation)
  X (15)..(16): X is any amino acid or no amino acid (deletion mutation)
  X (18)..(18): X is any amino acid or no amino acid (deletion mutation)
  X (24)..(24): X is any amino acid or no amino acid (deletion mutation)
  X (26)..(27): X is any amino acid or no amino acid (deletion mutation)
  X (32)..(37): X is any amino acid or no amino acid (deletion mutation)
  X (38)..(38): X is any amino acid or no amino acid; however, if X (Pos. 40) is N, X (Pos. 48) is A, X (Pos. 77) is P and X (Pos. 78) is P, then X (Pos. 38) is any amino acid except T or no amino acid
  X (40)..(40): X is any amino acid; however, if X (Pos. 38) is T, X (Pos. 48) is A, X (Pos. 77) is P and X (Pos. 78) is P, then X (Pos. 40) is any amino acid except N
  X (41)..(42): X is any amino acid or no amino acid (deletion mutation)
  X (44)..(44): X is any amino acid or no amino acid (deletion mutation)
  X (46)..(47): X is any amino acid or no amino acid (deletion mutation)
  X (48)..(48): X is any amino acid; however, if X (Pos. 38) is T, X (Pos. 40) is N, X (Pos. 77) is P and X (Pos. 78) is P, then X (Pos. 48) is any amino acid except A
  X (49)..(49): X is any amino acid or no amino acid (deletion mutation)
  X (51)..(52): X is any amino acid or no amino acid (deletion mutation)
  X (59)..(60): X is any amino acid or no amino acid (deletion mutation)
  X (63)..(64): X is any amino acid or no amino acid (deletion mutation)
  X (66)..(67): X is any amino acid or no amino acid (deletion mutation)
  X (69)..(70): X is any amino acid or no amino acid (deletion mutation)
  X (73)..(73): X is any amino acid or no amino acid (deletion mutation)
  X (76)..(76): X is any amino acid or no amino acid (deletion mutation)
  X (77)..(77): X is any amino acid; however, if X (Pos. 38) is T, X (Pos. 40) is N, X (Pos. 48) is A and X (Pos. 78) is P, then X (Pos. 77) is any amino acid except P
  X (78)..(78): X is any amino acid; however, if X (Pos. 38) is T, X (Pos. 40) is N, X (Pos. 48) is A and X (Pos. 77) is P, then X (Pos. 78) is any amino acid except P
  X (79)..(79): X is any amino acid or no amino acid (deletion mutation)
  X (82)..(83): X is any amino acid or no amino acid (deletion mutation)
  X (85)..(90): X is any amino acid or no amino acid (deletion mutation)
XXSXXXXXXXXLGSXXTXVCRGPXGXXTFRLXXXXXXXYXXXEXVXXXXPXXSEARFRXXSVXXGXXGXXRCXYYXXXXWSXXSXXXXXXV

SEQ ID NO: 18 - protein, 98 aa, Artificial Sequence
  LAIR-1 fragment
  X (1)..(2): X is any amino acid or no amino acid (deletion mutation)
  X (7)..(8): X is any amino acid or no amino acid (deletion mutation)
  X (10)..(17): X is any amino acid or no amino acid (deletion mutation)
  X (21)..(22): X is any amino acid or no amino acid (deletion mutation)
  X (24)..(24): X is any amino acid or no amino acid (deletion mutation)
  X (30)..(30): X is any amino acid or no amino acid (deletion mutation)
  X (32)..(33): X is any amino acid or no amino acid (deletion mutation)
  X (38)..(43): X is any amino acid or no amino acid (deletion mutation)
  X (44)..(44): X is any amino acid or no amino acid; however, if X (Pos. 46) is N, X (Pos. 54) is A, X (Pos. 83) is P and X (Pos. 84) is P, then X (Pos. 44) is any amino acid except T or no amino acid
  X (46)..(46): X is any amino acid or no amino acid; however, if X (Pos. 44) is T, X (Pos. 54) is A, X (Pos. 83) is P and X (Pos. 84) is P, then X (Pos. 46) is any amino acid except N
  X (47)..(48): X is any amino acid or no amino acid (deletion mutation)
  X (50)..(50): X is any amino acid or no amino acid (deletion mutation)
  X (52)..(53): X is any amino acid or no amino acid (deletion mutation)
  X (54)..(54): X is any amino acid or no amino acid; however, if X (Pos. 44) is T, X (Pos. 46) is N, X (Pos. 83) is P and X (Pos. 84) is P, then X (Pos. 54) is any amino acid except A
  X (55)..(55): X is any amino acid or no amino acid (deletion mutation)
  X (57)..(58): X is any amino acid or no amino acid (deletion mutation)
  X (65)..(66): X is any amino acid or no amino acid (deletion mutation)
  X (69)..(70): X is any amino acid or no amino acid (deletion mutation)
  X (72)..(73): X is any amino acid or no amino acid (deletion mutation)
  X (75)..(76): X is any amino acid or no amino acid (deletion mutation)
  X (79)..(79): X is any amino acid or no amino acid (deletion mutation)
  X (82)..(82): X is any amino acid or no amino acid (deletion mutation)
  X (83)..(83): X is any amino acid or no amino acid; however, if X (Pos. 44) is T, X (Pos. 46) is N, X (Pos. 54) is A and X (Pos. 84) is P, then X (Pos. 83) is any amino acid except P
  X (84)..(84): X is any amino acid or no amino acid; however, if X (Pos. 44) is is T, X (Pos. 46) is N, X  (Pos. 54) is A and X (Pos. 83) is P, then X  (Pos. 84) is any amino acid except P
  X (85)..(85): X is any amino acid or no amino acid (deletion mutation)
  X (88)..(89): X is any amino acid or no amino acid (deletion mutation)
  X (91)..(96): X is any amino acid or no amino acid (deletion mutation)
XXLPRPXXSXXXXXXXXLGSXXTXVCRGPXGXXTFRLXXXXXXXYXXXEXVXXXXPXXSEARFRXXSVXXGXXGXXRCXYYXXXXWSXXSXXXXXXVK

SEQ ID NO: 19 - protein, 41 aa, Artificial Sequence; identical to SEQ ID NO: 10
  LAIR-1 fragment
  X (1)..(1): X is T, L, G, I, R, K or no amino acid; however, if X (Pos. 3) is N, X (Pos. 11) is A, X (Pos. 40) is P and X (Pos. 41) is P, then X (Pos. 1) is L, G, I, R, K or no amino acid
  X (3)..(3): X is N, S or T; however, if X (Pos. 1) is T, X (Pos. 11) is A, X (Pos. 40) is P and X (Pos. 41) is P, then X (Pos. 3) is S or T
  X (4)..(5): X is any amino acid
  X (7)..(7): X is any amino acid
  X (9)..(10): X is any amino acid
  X (11)..(11): X is A, T, P, or V; however, if X (Pos. 1) is T, X (Pos. 3) is N, X (Pos. 40) is P and X (Pos. 41) is P, then X (Pos. 11) is T, P, or V
  X (12)..(12): X is any amino acid
  X (14)..(15): X is any amino acid
  X (22)..(23): X is any amino acid
  X (26)..(27): X is any amino acid
  X (29)..(30): X is any amino acid
  X (32)..(33): X is any amino acid
  X (36)..(36): X is any amino acid
  X (39)..(39): X is any amino acid
  X (40)..(40): X is P, S, A, or D; however, if X (Pos. 1) is T, X (Pos. 3) is N, X (Pos. 11) is A and X (Pos. 41) is P, then X (Pos.40) is S, A, or D
  X (41)..(41): X is P, R, or S; however, if X (Pos. 1) is T, X (Pos. 3) is N, X (Pos. 11) is A and X (Pos. 40) is P, then X (Pos. 41) is R, or S

SEQ ID NO: 20 - protein, 41 aa, Artificial Sequence; identical to SEQ ID NO: 10
  LAIR-1 fragment
  X (1)..(1): X is T or L; however, if X (Pos. 3) is N, X (Pos. 11) is A, X (Pos. 40) is P and X (Pos. 41) is P, then X (Pos. 1) is L
  X (3)..(3): X is N or S; however, if X (Pos. 1) is T, X (Pos. 11) is A, X (Pos. 40) is P and X (Pos. 41) is P, then X (Pos. 3) is S
  X (4)..(5): X is any amino acid
  X (7)..(7): X is any amino acid
  X (9)..(10): X is any amino acid
  X (11)..(11): X is A or T; however, if X (Pos. 1) is T, X (Pos. 3) is N, X (Pos. 40) is P and X (Pos. 41) is P, then X (Pos. 11) is T
  X (12)..(12): X is any amino acid
  X (14)..(15): X is any amino acid
  X (22)..(23): X is any amino acid
  X (26)..(27): X is any amino acid
  X (29)..(30): X is any amino acid
  X (32)..(33): X is any amino acid
  X (36)..(36): X is any amino acid
  X (39)..(39): X is any amino acid
  X (40)..(40): X is P or S; however, if X (Pos. 1) is T, X (Pos. 3) is N, X (Pos. 11) is A and X  (Pos. 41) is P, then X (Pos. 40) is S
  X (41)..(41): X is P or R; however, if X (Pos. 1) is T, X (Pos. 3) is N, X (Pos. 11) is A and X (Pos. 40) is P, then X (Pos. 41) is R

SEQ ID NO: 21 - protein, 98 aa, Artificial Sequence; identical to SEQ ID NO: 18
  LAIR-1 fragment
  X (1)..(2): X is any amino acid or no amino acid (deletion mutation)
  X (7)..(8): X is any amino acid or no amino acid (deletion mutation)
  X (10)..(17): X is any amino acid or no amino acid (deletion mutation)
  X (21)..(22): X is any amino acid or no amino acid (deletion mutation)
  X (24)..(24): X is any amino acid or no amino acid (deletion mutation)
  X (30)..(30): X is any amino acid or no amino acid (deletion mutation)
  X (32)..(33): X is any amino acid or no amino acid (deletion mutation)
  X (38)..(43): X is any amino acid or no amino acid (deletion mutation)
  X (44)..(44): X is T, L, G, I, R, K or no amino acid; however, if X (Pos. 46) is N, X (Pos. 54) is A, X (Pos. 83) is P and X (Pos.84) is P, then X (Pos. 44) is L, G, I, R, K or no amino acid
  X (46)..(46): X is N, S or T; however, if X (Pos. 44) is T, X (Pos. 54) is A, X (Pos. 83) is P and X (Pos. 84) is P, then X (Pos. 46) is S or T
  X (47)..(48): X is any amino acid or no amino acid (deletion mutation)
  X (50)..(50): X is any amino acid or no amino acid (deletion mutation)
  X (52)..(53): X is any amino acid or no amino acid (deletion mutation)
  X (54)..(54): X is A, T, P, or V; however, if X (Pos. 44) is T, X (Pos. 46) is N, X (Pos. 83) is P and X (Pos. 84) is P, then X (Pos. 54) is T, P, or V
  X (55)..(55): X is any amino acid or no amino acid (deletion mutation)
  X (57)..(58): X is any amino acid or no amino acid (deletion mutation)
  X (65)..(66): X is any amino acid or no amino acid (deletion mutation)
  X (69)..(70): X is any amino acid or no amino acid (deletion mutation)
  X (72)..(73): X is any amino acid or no amino acid (deletion mutation)
  X (75)..(76): X is any amino acid or no amino acid (deletion mutation)
  X (79)..(79): X is any amino acid or no amino acid (deletion mutation)
  X (82)..(82): X is any amino acid or no amino acid (deletion mutation)
  X (83)..(83): X4 is P, S, A, or D; however, if X (Pos. 44) is T, X (Pos. 46) is N, X (Pos. 54) is A and X (Pos. 84) is P, then X (Pos. 83) is S, A, or D
  X (84)..(84): X is P, R, or S; however, if X (Pos. 44) is T, X (Pos. 46) is N, X (Pos. 54) is A and X (Pos. 83) is P, then X (Pos. 84) is R or S
  X (85)..(85): X is any amino acid or no amino acid (deletion mutation)
  X (88)..(89): X is any amino acid or no amino acid (deletion mutation)
  X (91)..(96): X is any amino acid or no amino acid (deletion mutation)

SEQ ID NO: 22 - protein, 98 aa, Artificial Sequence; identical to SEQ ID NO: 18
  LAIR-1 fragment
  X (1)..(2): X is any amino acid or no amino acid (deletion)
  X (7)..(8): X is any amino acid or no amino acid (deletion)
  X (10)..(17): X is any amino acid or no amino acid (deletion)
  X (21)..(22): X is any amino acid or no amino acid (deletion)
  X (24)..(24): X is any amino acid or no amino acid (deletion)
  X (30)..(30): X is any amino acid or no amino acid (deletion)
  X (32)..(33): X is any amino acid or no amino acid (deletion)
  X (38)..(43): X is any amino acid or no amino acid (deletion)
  X (44)..(44): X is T or L; however, if X (Pos. 46) is N, X (Pos. 54) is A, X (Pos. 83) is P and X (Pos. 84) is P, then X (Pos. 44) is L
  X (46)..(46): X is N or S; however, if X (Pos. 44) is T, X (Pos. 54) is A, X (Pos. 83) is P and X (Pos. 84) is P, then X (Pos. 46) is S
  X (47)..(48): X is any amino acid or no amino acid (deletion)
  X (50)..(50): X is any amino acid or no amino acid (deletion)
  X (52)..(53): X is any amino acid or no amino acid (deletion)
  X (54)..(54): X is A or T; however, if X (Pos. 44) is T, X (Pos. 46) is N, X (Pos. 83) is P and X(Pos. 84) is P, then X (Pos. 54) is T
  X (55)..(55): X is any amino acid or no amino acid (deletion)
  X (57)..(58): X is any amino acid or no amino acid (deletion)
  X (65)..(66): X is any amino acid or no amino acid (deletion)
  X (69)..(70): X is any amino acid or no amino acid (deletion)
  X (72)..(73): X is any amino acid or no amino acid (deletion)
  X (75)..(76): X is any amino acid or no amino acid (deletion)
  X (79)..(79): X is any amino acid or no amino acid (deletion)
  X (82)..(82): X is any amino acid or no amino acid (deletion)
  X (83)..(83): X is P or S; however, if X (Pos. 44) is T, X (Pos. 46) is N, X (Pos. 54) is A and X (Pos. 84) is P, then X (Pos. 83) is S
  X (84)..(84): X is P or R; however, if X (Pos. 44) is T, X (Pos. 46) is N, X (Pos. 54) is A and X (Pos. 83) is P, then X (Pos. 84) is R
  X (85)..(85): X is any amino acid or no amino acid (deletion)
  X (88)..(89): X is any amino acid or no amino acid (deletion)
  X (91)..(96): X is any amino acid or no amino acid (deletion)

SEQ ID NO: 23 - protein, 98 aa, Artificial Sequence
  LAIR1ex+L aa
EDLPRPSISAEPGTVIPLGSHVTFVCRGPVGVQTFRLERESRSLYNDTEDVSQASPSESEARFRIDSVSEGNAGPYRCIYYKPPKWSEQSDYLELLVK

SEQ ID NO: 24 - DNA, 294 bp, Artificial Sequence
  LAIR1ex+L nucl
gaggacctgccaagacccagcatctccgcagaacctgggaccgtgattccactgggctcccacgtgacattcgtctgcagaggccccgtgggagtccagacttttaggctggagcgcgaatctcgaagtctgtacaacgacacagaggacgtgagccaggcctcaccaagcgagtccgaagctcggttcagaatcgactctgtcagtgaaggaaatgccggcccttacagatgcatctactataagccccctaaatggtcagagcagagcgattatctggaactgctggtgaag

SEQ ID NO: 25 - protein, 98 aa, Artificial Sequence
  LAIR1ex+LR aa
EDLPRPSISAEPGTVIPLGSHVTFVCRGPVGVQTFRLERESRSLYNDTEDVSQASPSESEARFRIDSVSEGNAGPYRCIYYKPRKWSEQSDYLELLVK

SEQ ID NO: 26 - DNA, 294 bp, Artificial Sequence
  LAIR1ex+LR nucl
gaggacctgccccgccctagcatctccgcagaaccagggaccgtgattcccctgggctcccacgtgacattcgtctgcaggggccccgtgggagtccagacttttaggctggagcgcgaatctcgaagtctgtacaacgacaccgaggacgtgagccaggcctcacctagcgagtccgaagctcggttcagaatcgactctgtcagtgaaggaaatgccggcccttacagatgcatctactataagccaagaaaatggtcagagcagagcgattatctggaactgctggtgaag

SEQ ID NO: 27 - protein, 98 aa, Artificial Sequence
  LAIR1ex+LS1 aa
EDLPRPSISAEPGTVIPLGSHVTFVCRGPVGVQTFRLERESRSLYSDTEDVSQASPSESEARFRIDSVSEGNAGPYRCIYYKPPKWSEQSDYLELLVK

SEQ ID NO: 28 - DNA, 294 bp, Artificial Sequence
  LAIR1ex+LS1 nucl
gaggacctgccaagacccagcatctccgcagaacctgggaccgtgattccactgggctcccacgtgacattcgtctgcagaggccccgtgggagtccagacttttaggctggagcgcgaatctcgaagtctgtactccgacacagaggacgtgagccaggcctcaccaagcgagtccgaagctcggttcagaatcgactctgtcagtgaaggaaacgccggcccttacagatgcatctactataagccccctaaatggtcagagcagagcgattatctggaactgctggtgaag

SEQ ID NO: 29 - protein, 98 aa, Artificial Sequence
  LAIR1ex+LS1R aa
EDLPRPSISAEPGTVIPLGSHVTFVCRGPVGVQTFRLERESRSLYSDTEDVSQASPSESEARFRIDSVSEGNAGPYRCIYYKPRKWSEQSDYLELLVK

SEQ ID NO: 30 - DNA, 294 bp, Artificial Sequence
  LAIR1ex+LS1R nucl
gaggacctgccccgccctagcatctccgcagaaccagggaccgtgattcccctgggctcccacgtgacattcgtctgcaggggccccgtgggagtccagacttttaggctggagcgcgaatctcgaagtctgtactccgacaccgaggacgtgagccaggcctcacctagcgagtccgaagctcggttcagaatcgactctgtcagtgaaggaaacgccggcccttacagatgcatctactataagccaagaaaatggtcagagcagagcgattatctggaactgctggtgaag

SEQ ID NO: 31 - protein, 98 aa, Artificial Sequence
  LAIR1ex+LS1S2R aa
EDLPRPSISAEPGTVIPLGSHVTFVCRGPVGVQTFRLERESRSLYSDTEDVSQASPSESEARFRIDSVSEGNAGPYRCIYYKSRKWSEQSDYLELLVK

SEQ ID NO: 32 - DNA, 294 bp, Artificial Sequence
  LAIR1ex+LS1S2R nucl
gaggacctgccccgccctagcatctccgcagaaccagggaccgtgattcccctgggctcccacgtgacattcgtctgcaggggccccgtgggagtccagacttttaggctggagcgcgaatctcgaagtctgtactccgacaccgaggacgtgagccaggcctcacctagcgagtccgaagctcggttcagaatcgactctgtcagtgaaggaaacgccggcccatacagatgcatctactataagagcagaaaatggtcagagcagagcgattatctggaactgctggtgaag

SEQ ID NO: 33 - protein, 98 aa, Artificial Sequence
  LAIR1ex+LS1T aa
EDLPRPSISAEPGTVIPLGSHVTFVCRGPVGVQTFRLERESRSLYSDTEDVSQTSPSESEARFRIDSVSEGNAGPYRCIYYKPPKWSEQSDYLELLVK

SEQ ID NO: 34 - DNA, 294 bp, Artificial Sequence
  LAIR1ex+LS1T nucl
gaggacctgccaagacccagcatctccgccgaacctgggactgtgattccactgggctcccacgtgaccttcgtctgcagaggccccgtgggagtccagaccttccggctggagcgcgaatctcgaagtctgtactccgacaccgaggacgtgagccagacatcacccagcgagtccgaagcccggttcagaatcgactctgtcagtgaaggaaacgctggcccttacagatgcatctactataagccccctaaatggtcagagcagagcgattatctggaactgctggtgaag

SEQ ID NO: 35 - protein, 98 aa, Artificial Sequence
  LAIR1ex+LS1TR aa
EDLPRPSISAEPGTVIPLGSHVTFVCRGPVGVQTFRLERESRSLYSDTEDVSQTSPSESEARFRIDSVSEGNAGPYRCIYYKPRKWSEQSDYLELLVK

SEQ ID NO: 36 - DNA, 294 bp, Artificial Sequence
  LAIR1ex+LS1TR nucl
gaggacctgcctagacctagcatctccgccgaaccagggactgtgattcccctgggctcccacgtgaccttcgtctgcagaggccccgtgggagtccagaccttccggctggagcgcgaatctcgaagtctgtactccgacaccgaggacgtgagccagacatcacctagcgagtccgaagcccggttcagaatcgactctgtcagtgaaggaaacgctggcccttacagatgcatctactataagccaagaaaatggtcagagcagagcgattatctggaactgctggtgaag

SEQ ID NO: 37 - protein, 98 aa, Artificial Sequence
  LAIR1ex+LS1TS2R aa
EDLPRPSISAEPGTVIPLGSHVTFVCRGPVGVQTFRLERESRSLYSDTEDVSQTSPSESEARFRIDSVSEGNAGPYRCIYYKSRKWSEQSDYLELLVK

SEQ ID NO: 38 - DNA, 294 bp, Artificial Sequence
  LAIR1ex+LS1TS2R nucl
gaggacctgcctagacctagcatctccgccgaaccagggactgtgattcccctgggctcccacgtgaccttcgtctgcagaggccccgtgggagtccagaccttccggctggagcgcgaatctcgaagtctgtactccgacaccgaggacgtgagccagacatcacctagcgagtccgaagcccggttcagaatcgactctgtcagtgaaggaaacgctggcccatacagatgcatctactataagagcagaaaatggtcagagcagagcgattatctggaactgctggtgaag

SEQ ID NO: 39 - protein, 98 aa, Artificial Sequence
  LAIR1ex+LS2R aa
EDLPRPSISAEPGTVIPLGSHVTFVCRGPVGVQTFRLERESRSLYNDTEDVSQASPSESEARFRIDSVSEGNAGPYRCIYYKSRKWSEQSDYLELLVK

SEQ ID NO: 40 - DNA, 294 bp, Artificial Sequence
  LAIR1ex+LS2R nucl
gaggacctgccccgccctagcatctccgcagaaccagggaccgtgattcccctgggctcccacgtgacattcgtctgcaggggccccgtgggagtccagacttttaggctggagcgcgaatctcgaagtctgtacaacgacaccgaggacgtgagccaggcctcacctagcgagtccgaagctcggttcagaatcgactctgtcagtgaaggaaatgccggcccatacagatgcatctactataagtctagaaaatggtcagagcagagcgattatctggaactgctggtgaag

SEQ ID NO: 41 - protein, 98 aa, Artificial Sequence
  LAIR1ex+LT aa
EDLPRPSISAEPGTVIPLGSHVTFVCRGPVGVQTFRLERESRSLYNDTEDVSQTSPSESEARFRIDSVSEGNAGPYRCIYYKPPKWSEQSDYLELLVK

SEQ ID NO: 42 - DNA, 294 bp, Artificial Sequence
  LAIR1ex+LT nucl
gaggacctgccaagacccagcatctccgccgaacctgggactgtgattccactgggctcccacgtgaccttcgtctgcagaggccccgtgggagtccagaccttccggctggagcgcgaatctcgaagtctgtacaacgacaccgaggacgtgagccagacatcacccagcgagtccgaagcccggttcagaatcgactctgtcagtgaaggaaatgctggcccttacagatgcatctactataagccccctaaatggtcagagcagagcgattatctggaactgctggtgaag

SEQ ID NO: 43 - protein, 98 aa, Artificial Sequence
  LAIR1ex+R aa
EDLPRPSISAEPGTVIPLGSHVTFVCRGPVGVQTFRLERESRSTYNDTEDVSQASPSESEARFRIDSVSEGNAGPYRCIYYKPRKWSEQSDYLELLVK

SEQ ID NO: 44 - DNA, 294 bp, Artificial Sequence
  LAIR1ex+R nucl
gaggacctgccccgccctagcatctccgcagaaccagggactgtgattcccctgggctcccacgtgaccttcgtctgcagaggccccgtgggagtccagaccttccggctggagcgcgaatctcgaagtacctacaacgacacagaggacgtgagccaggcctcacctagcgagtccgaagctcggttcagaatcgactctgtcagtgaaggaaatgccggcccttacagatgcatctactataagccaagaaaatggtcagagcagagcgattatctggaactgctggtgaag

SEQ ID NO: 45 - protein, 98 aa, Artificial Sequence
  LAIR1ex+S1 aa
EDLPRPSISAEPGTVIPLGSHVTFVCRGPVGVQTFRLERESRSTYSDTEDVSQASPSESEARFRIDSVSEGNAGPYRCIYYKPPKWSEQSDYLELLVK

SEQ ID NO: 46 - DNA, 294 bp, Artificial Sequence
  LAIR1ex+S1 nucl
gaggacctgccaagacccagcatctccgcagaacctgggactgtgattccactgggctcccacgtgaccttcgtctgcagaggccccgtgggagtccagaccttccggctggagcgcgaatctcgaagtacctactccgacacagaggacgtgagccaggcctcacccagcgagtccgaagctcggttcagaatcgactctgtcagtgaaggaaacgccggcccttacagatgcatctactataagccccctaaatggtcagagcagagcgattatctggaactgctggtgaag

SEQ ID NO: 47 - protein, 98 aa, Artificial Sequence
  LAIR1ex+S1R aa
EDLPRPSISAEPGTVIPLGSHVTFVCRGPVGVQTFRLERESRSTYSDTEDVSQASPSESEARFRIDSVSEGNAGPYRCIYYKPRKWSEQSDYLELLVK

SEQ ID NO: 48 - DNA, 294 bp, Artificial Sequence
  LAIR1ex+S1R nucl
gaggacctgccccgccctagcatctccgcagaaccagggactgtgattcccctgggctcccacgtgaccttcgtctgcagaggccccgtgggagtccagaccttccggctggagcgcgaatctcgaagtacctactccgacacagaggacgtgagccaggcctcacctagcgagtccgaagctcggttcagaatcgactctgtcagtgaaggaaacgccggcccttacagatgcatctactataagccaagaaaatggtcagagcagagcgattatctggaactgctggtgaag

SEQ ID NO: 49 - protein, 98 aa, Artificial Sequence
  LAIR1ex+S1S2R aa
EDLPRPSISAEPGTVIPLGSHVTFVCRGPVGVQTFRLERESRSTYSDTEDVSQASPSESEARFRIDSVSEGNAGPYRCIYYKSRKWSEQSDYLELLVK

SEQ ID NO: 50 - DNA, 294 bp, Artificial Sequence
  LAIR1ex+S1S2R nucl
gaggacctgccccgccctagcatctccgcagaaccagggactgtgattcccctgggctcccacgtgaccttcgtctgcagaggccccgtgggagtccagaccttccggctggagcgcgaatctcgaagtacctactccgacacagaggacgtgagccaggcctcacctagcgagtccgaagctcggttcagaatcgactctgtcagtgaaggaaacgccggcccatacagatgcatctactataagagcagaaaatggtcagagcagagcgattatctggaactgctggtgaag

SEQ ID NO: 51 - protein, 98 aa, Artificial Sequence
  LAIR1ex+S1T aa
EDLPRPSISAEPGTVIPLGSHVTFVCRGPVGVQTFRLERESRSTYSDTEDVSQTSPSESEARFRIDSVSEGNAGPYRCIYYKPPKWSEQSDYLELLVK

SEQ ID NO: 52 - DNA, 294 bp, Artificial Sequence
  LAIR1ex+S1T nucl
gaggacctgccaagacccagcatctccgccgaacctgggactgtgattccactgggctcccacgtgaccttcgtctgcagaggccccgtgggagtccagaccttccggctggagcgcgaatctcgaagtacctactccgacacagaggacgtgagccagacctcacccagcgagtccgaagcccggttcagaatcgactctgtcagtgaaggaaacgctggcccttacagatgcatctactataagccccctaaatggtcagagcagagcgattatctggaactgctggtgaag

SEQ ID NO: 53 - protein, 98 aa, Artificial Sequence
  LAIR1ex+S2 aa
EDLPRPSISAEPGTVIPLGSHVTFVCRGPVGVQTFRLERESRSTYNDTEDVSQASPSESEARFRIDSVSEGNAGPYRCIYYKSPKWSEQSDYLELLVK

SEQ ID NO: 54 - DNA, 294 bp, Artificial Sequence
  LAIR1ex+S2 nucl
gaggacctgcccagacctagcatctccgcagaaccagggactgtgattcccctgggctcccacgtgaccttcgtctgcagaggccccgtgggagtccagaccttccggctggagcgcgaatctcgaagtacctacaacgacacagaggacgtgagccaggcctcacctagcgagtccgaagctcggttcagaatcgactctgtcagtgaaggaaatgccggcccttacagatgcatctactataagtctccaaaatggtcagagcagagcgattatctggaactgctggtgaag

SEQ ID NO: 55 - protein, 98 aa, Artificial Sequence
  LAIR1ex+S2R aa
EDLPRPSISAEPGTVIPLGSHVTFVCRGPVGVQTFRLERESRSTYNDTEDVSQASPSESEARFRIDSVSEGNAGPYRCIYYKSRKWSEQSDYLELLVK

SEQ ID NO: 56 - DNA, 294 bp, Artificial Sequence
  LAIR1ex+S2R nucl
gaggacctgccccgccctagcatctccgcagaaccagggactgtgattcccctgggctcccacgtgaccttcgtctgcagaggccccgtgggagtccagaccttccggctggagcgcgaatctcgaagtacctacaacgacacagaggacgtgagccaggcctcacctagcgagtccgaagctcggttcagaatcgactctgtcagtgaaggaaatgccggcccatacagatgcatctactataagtctagaaaatggtcagagcagagcgattatctggaactgctggtgaag

SEQ ID NO: 57 - protein, 98 aa, Artificial Sequence
  LAIR1ex+T aa
EDLPRPSISAEPGTVIPLGSHVTFVCRGPVGVQTFRLERESRSTYNDTEDVSQTSPSESEARFRIDSVSEGNAGPYRCIYYKPPKWSEQSDYLELLVK

SEQ ID NO: 58 - DNA, 294 bp, Artificial Sequence
  LAIR1ex+T nucl
gaggacctgccaagacccagcatctccgccgaacctgggactgtgattccactgggctcccacgtgaccttcgtctgcagaggccccgtgggagtccagaccttccggctggagcgcgaatctcgaagtacctacaacgacacagaggacgtgagccagacctcacccagcgagtccgaagcccggttcagaatcgactctgtcagtgaaggaaatgctggcccttacagatgcatctactataagccccctaaatggtcagagcagagcgattatctggaactgctggtgaag

SEQ ID NO: 59 - protein, 98 aa, Artificial Sequence
  LAIR1ex+TS2R aa
EDLPRPSISAEPGTVIPLGSHVTFVCRGPVGVQTFRLERESRSTYNDTEDVSQTSPSESEARFRIDSVSEGNAGPYRCIYYKSRKWSEQSDYLELLVK

SEQ ID NO: 60 - DNA, 294 bp, Artificial Sequence
  LAIR1ex+TS2R nucl
gaggacctgcctagacctagcatctccgccgaaccagggactgtgattcccctgggctcccacgtgaccttcgtctgcagaggccccgtgggagtccagaccttccggctggagcgcgaatctcgaagtacctacaacgacacagaggacgtgagccagacctcacctagcgagtccgaagcccggttcagaatcgactctgtcagtgaaggaaatgctggcccatacagatgcatctactataagtctagaaaatggtcagagcagagcgattatctggaactgctggtgaag

SEQ ID NO: 61 - protein, 98 aa, Artificial Sequence
  MGC1/MGC32_EXON aa
EDLPRPSISAAEGTVIPLGSHVTFVCRGPVGVQTFRLEKDSRSIYNDTENVSQPSPSESEARFRIDSVSEGNAGLYRCVYYKAPKWSAQSDYLELLVK

SEQ ID NO: 62 - DNA, 294 bp, Artificial Sequence
  MGC1/MGC32_EXON nucl
gaagatctgcccagaccctccatctcggctgccgaaggcaccgtgatccccctggggagccatgtgactttcgtgtgccggggcccggttggggttcaaacattccgcctggagaaggacagtagatccatatacaatgatactgaaaatgtgtctcaacctagtccatctgagtcagaggccagatttcgcattgactcagtgagtgaaggaaatgccggactttatcggtgcgtctattataaggcccctaaatggtctgcgcagagtgattacctggagctgctggtgaaa

SEQ ID NO: 63 - protein, 98 aa, Artificial Sequence
  MGC2_EXON aa
EHLPRPSISPEPGTVITLGSHVTFVCRGPVGVQTFRLEKDSRSTYNDTEDVSQPSPSESEARFRIDSVSEGYAGLYRCLYYKPPKWSEQSDYLELLVK

SEQ ID NO: 64 - DNA, 294 bp, Artificial Sequence
  MGC2_EXON nucl
gagcatctgcccagaccctccatctcgcctgagccaggcaccgtgatcaccctggggagccatgtgactttcgtgtgccggggcccggttggggttcaaacattccgcctggagaaggacagtagatccacatacaatgatactgaagatgtgtctcaacctagtccatctgagtcagaggccagattccgcattgactcagtaagtgaaggatatgccgggctttatcgctgcctctattataagccccctaaatggtctgagcagagtgactacctggagctgctggtgaaa

SEQ ID NO: 65 - protein, 96 aa, Artificial Sequence
  MGC4_EXON aa
EDLPRPSISAEPDTVIPLGSHVTFVCRGPVGVHTFRLERGWRYNDTEDVSQAGPSESEARFRIDSVREGNAGLYRCIYYIAPKWSEQSDYLELRVK

SEQ ID NO: 66 - DNA, 288 bp, Artificial Sequence
  MGC4_EXON nucl
gaagatctgcccagaccctccatctcggctgagccagacaccgtaatccccctggggagccatgtgactttcgtgtgccggggcccggttggggttcacacattccgcctggagagggggtggaggtacaacgacactgaagatgtgtctcaagctggtccatctgagtcagaggccagattccgcattgactcggtaagggaaggaaatgccgggctttatcgatgcatctattacatagcccctaaatggtctgagcagagtgactacctggagctgcgggtgaaa

SEQ ID NO: 67 - protein, 96 aa, Artificial Sequence
  MGC5/MGC29_EXON aa
EDLPRPSISAEPGTVIPLGSHVTFVCRGPVGVHTFRLERGWRYNDTEDVSQAGPSQSEARFRIDSVREGNAGLYRCLYYIPPKWSEQSDYLELRVK

SEQ ID NO: 68 - DNA, 288 bp, Artificial Sequence
  MGC5/MGC29_EXON nucl
gaagatctgcccagaccctccatctcggctgagccaggcaccgtgatccccctggggagccatgtgactttcgtgtgccggggccccgttggggttcacacattccgcctggagagggggtggagatacaacgacactgaagatgtgtctcaagctggtccatctcagtcagaggccagattccgcattgactcggtaagggaaggaaatgccgggctttatcgatgcctctattacataccccctaaatggtctgagcagagtgactacctggaactgcgggtgaaa

SEQ ID NO: 69 - protein, 98 aa, Artificial Sequence
  MGC7/MGC37_EXON aa
DDLPRPSISPEPGTVIPLGSHVTFVCRGPVGVQTFRLEKDRRSTYNDTEDVSQPSPSESEARFRIDSVTEGNAGLYRCVYYKPPKWSDQSDFLELLVK

SEQ ID NO: 70 - DNA, 294 bp, Artificial Sequence
  MGC7/MGC37_EXON nucl
gatgatctgcccagaccctctatctcgcctgagccaggcaccgtgatccccctggggagccatgtgactttcgtgtgtcggggcccggttggggttcaaacattccgcctggagaaggacagaagatccacatacaatgatactgaagatgtgtctcaacctagtccatctgagtcagaggccagattccgcattgactcagtaactgaaggaaatgccgggctttatcgctgcgtctattataagccccctaaatggtctgaccagagtgacttcctggagttgctggtgaag

SEQ ID NO: 71 - protein, 98 aa, Artificial Sequence
  MGC17_EXON aa
EDLPRPSISAEEGTVIPLGSRLTFVCRGPVGVHTFRLERDRRSTYNDTEDVSHPSPSESEARFRIDSVSEGNAGLYRCVYYKSPEWSKQSDYLELLVK

SEQ ID NO: 72 - DNA, 294 bp, Artificial Sequence
  MGC17_EXON nucl
gaagatctgcccagaccctccatctcggctgaggaaggcaccgtgattcccctggggagccgtctgactttcgtgtgccggggcccggttggggttcacacattccgcctggagagggaccgtagatccacatacaatgatactgaagatgtgtctcaccctagtccatctgagtctgaggccagatttcgcattgactcagtgagtgaaggaaatgccgggctttatcgctgcgtctattataagtcccctgaatggtctaagcagagtgattacctggagctgctggtgaaa

SEQ ID NO: 73 - protein, 98 aa, Artificial Sequence
  MGC26_EXON aa
EDLPRPSISPEPATVIPLGSHVTIVCRGPVGVETFRLQKESRSLYNDTEDVSQPSPSESEARFRIDSVSEGHGGLYRCLYYKSSKWSEQSDYLEMLVK

SEQ ID NO: 74 - DNA, 294 bp, Artificial Sequence
  MGC26_EXON nucl
gaagatctgcccagaccctccatctcgccggagccagccaccgtgatccccctggggagccatgtgactatcgtgtgccggggcccggttggggttgaaacattccgcctgcagaaggagagtagatccctgtacaatgacactgaagatgtgtctcaacctagtccatctgagtcagaggccagattccgcattgactcagtaagtgaagggcatggcgggctttatcgctgcctctattataagtcttctaaatggtctgagcagagtgactacctggagatgctggtgaaa

SEQ ID NO: 75 - protein, 98 aa, Artificial Sequence
  MGC28/MGC33_EXON aa
EDLPRPTISAETGTVISLGSHVTFVCRGPLGVQTFRLERESRSRYSETEDVSQVGPSESEARFRIDSVSEGNAGLYRCIYYKPPKWSEQSDYLELRVK

SEQ ID NO: 76 - DNA, 294 bp, Artificial Sequence
  MGC28/MGC33_EXON nucl
gaagatctgcccagacccaccatctcggctgagacaggcaccgtgatctccctggggagccatgtgactttcgtgtgccggggcccacttggggtgcaaacattccgcctggagagggagagtaggtccagatacagtgaaactgaagatgtgtctcaagttggtccatctgagtcagaggccagattccgcattgactcagtgagtgaaggaaatgccgggctttatcgatgcatctattacaaaccccctaaatggtctgagcagagtgactacctggagctgcgggtgaaa

SEQ ID NO: 77 - protein, 98 aa, Artificial Sequence
  MGC34_EXON aa
EDLPRPSISAEPGTVIPLGSHVTFVCRGPVGIHTFRLERESRSLYTETEDVTQVSPSESEARFRIESVTEGNAGLYRCVYYKPPKWSEQSDYLELLVK

SEQ ID NO: 78 - DNA, 294 bp, Artificial Sequence
  MGC34_EXON nucl
gaagatctgcccagaccctccatctcggctgagccaggcaccgtgatccccctggggagtcatgtgaccttcgtgtgccggggcccggttgggattcacacattccgcctggagagggagagtagatccctatacactgaaactgaagatgtgactcaagtaagtccttctgagtcagaggccagattccgcattgagtcagtaactgaaggaaatgccgggctttatcgctgcgtctattataagccccctaaatggtctgagcagagtgactacctggagctgctggtgaaa

SEQ ID NO: 79 - protein, 96 aa, Artificial Sequence
  MGC35_EXON aa
EDLPRPSISAEPGSVIPLGSLVTFVCRGPVGVHTFRLERGWTYNDTEDVSQAGPSESEARFRMDSVREGNAGLYRCIYYKPPKWSEQSAYLELRVK

SEQ ID NO: 80 - DNA, 288 bp, Artificial Sequence
  MGC35_EXON nucl
gaagatctgcccagaccctccatctcggctgagccaggctccgtgatccccctggggagccttgtgactttcgtgtgccggggcccggttggggttcacacattccgcctcgagagggggtggacatacaacgacactgaagatgtgtctcaagctggtccatctgagtcagaggccagattccgcatggactcggtaagggaaggaaatgccgggctttatcgatgcatctattacaaaccccctaaatggtctgagcagagtgcctacctggaactgcgggtgaaa

SEQ ID NO: 81 - protein, 97 aa, Artificial Sequence
  MGC36_EXON aa
EEDLPRPSISAEPDTVIPLGSHVTFVCRGPVGVHTFRLERGWRYNDTEDVSQAGPSESEARFRIDSVREGNAGLYRCIYYIAPKWSEQSDYLELRVK

SEQ ID NO: 82 - DNA, 291 bp, Artificial Sequence
  MGC36_EXON nucl
gaagaagatctgcccagaccctccatctcggctgagccagacaccgtaatccccctggggagccatgtgactttcgtgtgccggggcccggttggggttcacacattccgcctggagagggggtggaggtacaacgacactgaagatgtgtctcaagctggtccatctgagtcagaggccagattccgcattgactcggtaagggaaggaaatgccgggctttatcgatgcatctattacatagcccctaaatggtctgagcagagtgactacctggagctgcgggtgaaa

SEQ ID NO: 83 - protein, 97 aa, Artificial Sequence
  MGD21_EXON aa
DLPRPSISAEPGTVIPLGSHVTFVCRGPVGVQTFRLERERNYLYSDTEDVSQTSPSESEARFRIDSVNAGNAGLFRCIYYKSRKWSEQSDYLELVVK

SEQ ID NO: 84 - DNA, 291 bp, Artificial Sequence
  MGD21_EXON nucl
gatctgcccagaccctccatctcggctgagccgggcaccgtgatccccctggggagccatgtgactttcgtgtgccggggcccggttggggttcaaacattccgcctggagagggagaggaattatttatacagtgatactgaagatgtgtctcaaactagtccatctgagtcggaggccagattccgcattgactcagtaaatgcaggcaatgccgggctttttcgctgcatctattacaagtcccgtaaatggtctgagcagagtgactacctggagctggtggtgaaa

SEQ ID NO: 85 - protein, 98 aa, Artificial Sequence
  MGD23_EXON aa
EDLPRPSLSAEPGTVIPLGSHVTFVCRGPAGVETFRLERESRFTYNDTEDVSQASPSESEARFRIDSVSEGNAGPYRCLYYKARKWSDQSDYLELLVK

SEQ ID NO: 86 - DNA, 294 bp, Artificial Sequence
  MGD23_EXON nucl
gaagatctgcccagaccctccctctcggctgaaccaggcaccgtgatccccctggggagtcacgtgactttcgtgtgccggggcccggctggggtcgaaacattccgcctggagagggagagtagattcacttacaacgatactgaagatgtgtctcaagcgagtccatctgagtcagaggccagattccgcattgactcagtaagtgaaggaaatgccgggccttatcgctgcctctattataaggcccgtaaatggtctgaccagagtgactacttggaattgctggtgaag

SEQ ID NO: 87 - protein, 98 aa, Artificial Sequence
  MGD30_EXON aa
EKLPRPSISAEPGTVIPLGSRVTFVCRGPVGVQTFRLERETSFTYNDTEDVSQVSPSESEARFRIDSVSEGYAGPYRCVYYKAPKWSEQSDYLDLLVK

SEQ ID NO: 88 - DNA, 294 bp, Artificial Sequence
  MGD30_EXON nucl
gaaaaactgcccagaccctccatctcggctgagccgggcaccgtgatccccctggggagccgtgtgactttcgtgtgccggggcccggttggggttcaaacattccgcctagagagggagactagctttacatataatgatactgaagatgtgtctcaggttagtccgtctgagtcagaggccagattccgcattgactcagtgagtgagggatatgccgggccttatcgctgcgtctattataaggcccctaagtggtccgagcagagtgactacctggacctgctggtgaaa

SEQ ID NO: 89 - protein, 98 aa, Artificial Sequence
  MGD33_EXON aa
EKLPRPSISAEPGTVIPLGSRVTFVCRGPVGVQTFRLERETRSTYNDTEDVSQVSPSESEARFRIDSVSEGYAGPYRCVYYKAPKWSEQSDYLDLLVK

SEQ ID NO: 90 - DNA, 294 bp, Artificial Sequence
  MGD33_EXON nucl
gaaaaactgcccagaccctccatctcggctgagccgggcaccgtgatccccctggggagccgtgtgactttcgtgtgccggggcccggttggggttcaaacattccgcctagagagggagactagatctacatataatgatactgaagatgtgtctcaggttagtccgtctgagtcagaggccagattccgcattgactcagtgagtgagggatatgccgggccttatcgctgcgtctattataaggcccctaagtggtccgagcagagtgactacctggacctgctggtgaaa

SEQ ID NO: 91 - protein, 98 aa, Artificial Sequence
  MGD34_EXON aa
EDLPRPSISAEPGTVIPLGSHVTFVCRGPVGVQTFRLERERNYLYSDTEDVSQTSPSESEARFRIDSVNAGNAGLFRCIYYKSRKWSEQSDYLELVVK

SEQ ID NO: 92 - DNA, 294 bp, Artificial Sequence
  MGD34_EXON nucl
gaagatctgcccagaccctccatctcggctgagccgggcaccgtgatccccctggggagccatgtgactttcgtgtgccggggcccggttggggttcaaacattccgcctggagagggagaggaattatttatacagtgatactgaagatgtgtctcaaactagtccatctgagtcggaggccagattccgcattgactcagtaaatgcaggcaatgccgggctttttcgctgcatctattacaagtcccgtaaatggtctgagcagagtgactacctggagctggtggtgaaa

SEQ ID NO: 93 - protein, 97 aa, Artificial Sequence
  MGD35_EXON aa
NLPRPSLSAEPGTVIPLGSPVTFVCRGPVGVHTFRLERAGRSTYNDTEDVSHPSPSESEARFRIDSVSEGNAGPYRCVYYKSSKWSEESYCLDLLVK

SEQ ID NO: 94 - DNA, 291 bp, Artificial Sequence
  MGD35_EXON nucl
aatttgcccagaccctccctctcggcggagccaggcaccgtgatccccctggggagccctgtgactttcgtgtgccggggcccggttggggttcacacattccgcctggagagggcgggtagatccacatacaatgatactgaagatgtgtctcatcctagtccatctgagtcagaggccagattccgcattgactcagtgagtgagggaaatgccgggccttatcgctgcgtctattataagtcctctaaatggtccgaggagagttactgcctggacctgctggtcaaa

SEQ ID NO: 95 - protein, 98 aa, Artificial Sequence
  MGD39_EXON aa
DDLPRPSISAEPGTVIPLGSHVTFVCRGPIGVQTFRLERERRSLYSDTEDVSQVSPFASEARFRIDSVSEGNAGPYRCIYYKDRKWSDQSDYLELLVK

SEQ ID NO: 96 - DNA, 294 bp, Artificial Sequence
  MGD39_EXON nucl
gacgatctgcccagaccctccatctcggctgagccaggcaccgtgatccccctggggagccatgtgaccttcgtgtgccggggcccaattggggttcaaacattccgcctggagagggagagaagatccttatacagtgatactgaagatgtgtctcaagttagtccatttgcgtcagaggccagattccgcattgactcagtaagtgaaggaaatgccgggccatatcgctgcatctattataaggaccggaaatggtctgaccagagtgactacctggagttgctggtgaaa

SEQ ID NO: 97 - protein, 98 aa, Artificial Sequence
  MGD41_EXON aa
EDLPRPSLSAEPGTVVPLGSHVTFVCRGPVGVQTFRLERESRSTYNDTEDVSQPSPFESEARFRIDSVSEGNAGPYRCIYYKSPKWSDQSDYVELLVK

SEQ ID NO: 98 - DNA, 294 bp, Artificial Sequence
  MGD41_EXON nucl
gaagatctgcccagaccctccctctcggctgagccaggcaccgtggtccccctggggagccatgtgactttcgtgtgccggggcccggttggggttcaaacattccgcctggagagggagagcagatccacatacaatgatactgaagatgtgtctcaacctagtccatttgagtcagaggccagatttcgcattgactcagtaagtgaaggaaatgccgggccttatcgctgcatctattataagtcccctaaatggtctgaccagagtgactacgtggagttgctggtgaaa

SEQ ID NO: 99 - protein, 98 aa, Artificial Sequence
  MGD47_EXON aa
GDLPRPSISAEPGTAIPLGSQVTFVCRGPIGVQTFRLERESRALYNDSEDVSQVSPSASEARFRIDSVSEGNAGPYRCIYYKARRWSDQSDYLELLVK

SEQ ID NO: 100 - DNA, 294 bp, Artificial Sequence
  MGD47_EXON nucl
ggagatctgcccagaccctccatctcggctgagccaggcaccgcgatccccctggggagccaagtgactttcgtgtgccggggcccaattggggttcaaacattccgcctggagagggagagtcgcgccttatataatgattctgaagatgtgtctcaagttagtccatctgcgtcagaggccagattccgcattgactcagtaagtgaaggcaatgccgggccttatcgctgtatctattataaggcccgcagatggtctgaccagagtgactatttggagttgttggtgaaa

SEQ ID NO: 101 - protein, 98 aa, Artificial Sequence
  MGD55_EXON aa
DDLPRPSISAEPGTVIPLGSHVTFVCRGPIGVQTFRLERESRSLYSDTEDVSQVSPFASEARFRIDSVSEGNAGPYRCIYYKDRKWSDQSDYLELLVK

SEQ ID NO: 102 - DNA, 294 bp, Artificial Sequence
  MGD55_EXON nucl
gacgatctgcccagaccctccatctcggctgagccaggcaccgtgatccccctggggagccatgtgactttcgtgtgccggggcccaattggggttcaaacattccgcctggagagggagagtagatccttatacagtgatactgaagatgtgtctcaagttagtccatttgcgtcagaggccagattccgcattgactcagtaagtgaaggaaatgccgggccatatcgctgcatctattataaggaccggaaatggtctgaccagagtgactacctggagttgctggtgaaa

SEQ ID NO: 103 - protein, 98 aa, Artificial Sequence
  MGD56_EXON aa
KDLPRPSLSAEPGTVIPLGSHVTFVCRGPVGVQTFRLQRESRSLYNDTEDVSHPSPSESEARFRIDSVSEGNAGPYRCVYYKSSKWSEESDCLELLVK

SEQ ID NO: 104 - DNA, 294 bp, Artificial Sequence
  MGD56_EXON nucl
aaagatttgcccagaccctccctctcggctgagccaggcaccgtgatccccctggggagtcatgtgactttcgtgtgccggggcccggttggggttcagacattccgcctgcagagggagagtagatccctttacaatgatactgaagatgtgtctcatcctagtccatctgagtcagaggccagattccgcattgactcagtgagtgagggaaatgccgggccttatcgctgcgtctattataagtcctctaaatggtccgaggagagtgactgcctggagctgctggtcaaa

SEQ ID NO: 105 - protein, 367 aa, Plasmodium falciparum
MKDHYINILLFALPLNILVYNQRNYYITPRHTETNRSLCECELYSPTNYDSDPEMKRVMQQFVDRTTQRFHEYDESLQSKRKQCKDQCDKEIQKIILKDKIEKEFTEKLSTLQTDITTKDIPTCVCEKSLADKMEKVCLKCAQNLGGIVAPSTGVLGEIAALAVNAWKTTALKNAIAAAQKAGDAAGKIAGESKGVETIIGILEQYYSIYELKGTPLKSFFATTHYTDISNIATVIDTELNTSCGLNSLANQAICGLRTKLGLVAKPGQVMVTQKEAITKMITNVVHKSEITAEAAKTEVAATKTAAAIKMNTEAIEAATTPYYTPIIASIVAIVVIVLIMVIIYLILRYRRKKKMKKKLQYIKLLN

SEQ ID NO: 106 - DNA, 1104 bp, Plasmodium falciparum
atgaaagaccattatattaatatattattgtttgctcttccattaaatatattggtatataatcaaaggaactattacattacaccacgtcatacagaaaccaacagatctttatgtgaatgtgaattatattcacctacgaactatgatagtgatcccgaaatgaaaagggtaatgcaacaatttgtggatcgtacaacacaacgatttcacgaatatgatgaaagtttgcaaagtaaacgaaagcaatgcaaagatcaatgcgataaagaaatccaaaaaattatattaaaagataaaatcgaaaaggaatttacagaaaaattatcaacattacaaacagatataacgactaaagacatacccacctgtgtttgcgaaaaatccttggcggacaaaatggaaaaagtatgcttgaaatgtgcacaaaatttgggaggtattgttgcaccctctacaggagtattaggcgaaattgctgcacttgctgtaaatgcctggaaaactacggcacttaagaacgctattgcggcagctcaaaaagcaggtgatgcggccggtaaaattgcgggggaatccaagggtgttgaaacaattattggaatattagaacaatattactctatatatgagttaaaaggaacaccattgaaatccttttttgctacaacgcattatactgatatctcaaatattgctactgttattgatacggaattgaatacgtcttgtgggttgaattccttagctaatcaggctatttgcggtcttcgtacgaaattaggtcttgttgcaaaacctggtcaagttatggttacacagaaagaagctataacaaagatgataaccaacgttgttcataaatctgaaattactgctgaagctgcaaagactgaggtggctgcaactaaaacagcagcagctataaagatgaacacagaagctatagaagctgcaactactccttactatactcctataatagcatccatcgttgcaatagtggtcatagttttaattatggtgataatttatttgattttacgttatcgaagaaaaaaaaaaatgaagaaaaaactccaatatataaaattattaaattaa

SEQ ID NO: 107 - protein, 378 aa, Plasmodium falciparum
MKFNYTNIILFSLSLNILLLSSRVYNKRNHKSIILHTSNENPIKTHRSLCECELYSPTNYDSDPEMKRVMQQFHDRTTQRFHEYDERMKTTRQECKEQCDKEIQKIILKDRLEKELMDKFATLHTDIQSDAIPTCVCEKSLADKTEKFCLNCGVQLGGGVLQASGLLGGIGQLGLDAWKAAALVTAKELAEKAGAAAGLKAGDIHGMKIVIEGLKALKVDTLKSGIFNSFVNNSHYTEVTGLAIAIDTEMNEVCSATYIGIHPICVVREKLGVIPKAGGTMVKQKDAITNVLKQALEKATQSAEALSETTAEDVAAKLTAQKTGAINTIFMSNQTAIIASIVAIVVIVLIMVIIYLILRYRRKKKMKKKLQYIKLLEE

SEQ ID NO: 108 - DNA, 1137 bp, Plasmodium falciparum
atgaagttcaattacactaatataatattattttccctttcattaaatatattgttattatcatcacgggtatacaataaaaggaatcataaaagcattatacttcatacatcaaacgaaaacccaataaaaacacatagatcattatgcgaatgcgaattatattcacctacgaactatgatagtgatcccgaaatgaaaagggtaatgcaacaatttcatgatcgtacaacacaacgatttcacgaatacgacgaaaggatgaaaactacacgccaagaatgtaaagaacaatgcgataaagaaatacaaaaaattattttaaaagacagattagaaaaagaattaatggacaaatttgccacactacacacagatatacaaagtgatgctattccaacatgtgtttgcgaaaagtcgttagcagataaaacagaaaaattttgtctgaactgtggggtgcaactaggaggtggtgtgttgcaagcttcgggtttattaggaggaattggtcaacttgggctagatgcatggaaagcagccgcgttggtaactgctaaggaacttgccgaaaaagccggtgctgcagcaggtcttaaagcaggtgatatccatggtatgaaaatagttattgaaggattaaaagcattgaaagtagatacattaaaatctggaatatttaattcctttgttaataacagccattatactgaagtcacagggcttgctattgctattgatactgaaatgaatgaggtgtgttcagcgacgtatattggtattcatcctatctgcgttgttcgtgagaaattaggtgtaataccaaaggctggtggaacaatggttaaacagaaagatgctataacaaatgtgttaaagcaagctcttgaaaaagctacacaaagtgctgaagcactttctgagactactgctgaagacgttgctgctaaactcacagctcaaaagacgggtgcgataaatactatatttatgagtaatcagactgctattattgcttccatcgttgcaatagtagttatagttttaattatggtgataatatatttaattttacgttatcgacgaaaaaaaaaaatgaagaaaaaactccaatatatcaaattattagaagaatag

SEQ ID NO: 109 - protein, 4 aa, Artificial Sequence
  enterokinase target site
DDDK

SEQ ID NO: 110 - protein, 5 aa, Artificial Sequence
  factor Xa target site
IEDGR

SEQ ID NO: 111 - protein, 6 aa, Artificial Sequence
  thrombin target site
LVPRGS

SEQ ID NO: 112 - protein, 7 aa, Artificial Sequence
  protease TEV target site
ENLYFQG

SEQ ID NO: 113 - protein, 8 aa, Artificial Sequence
  PreScission protease target site
LEVLFQGP

SEQ ID NO: 114 - protein, 4 aa, Artificial Sequence
  furin target site
  misc_feature (2)..(2): Xaa can be any amino acid
  X (3)..(3): X is R or K
RXXR

SEQ ID NO: 115 - protein, 5 aa, Artificial Sequence
  linker
GGGGG

SEQ ID NO: 116 - protein, 4 aa, Artificial Sequence
  linker
GGGG

SEQ ID NO: 117 - protein, 5 aa, Artificial Sequence
  linker
GGGGS

SEQ ID NO: 118 - protein, 4 aa, Artificial Sequence
  linker
EQLE

SEQ ID NO: 119 - protein, 4 aa, Artificial Sequence
  linker
TEWT

SEQ ID NO: 120 - protein, 8 aa, Artificial Sequence
  MGC1 CDRH1 aa
GFNFRKSW

SEQ ID NO: 121 - protein, 8 aa, Artificial Sequence
  MGC1 CDRH2 aa
IREDGSES

SEQ ID NO: 122 - protein, 139 aa, Artificial Sequence
  MGC1 CDRH3 aa
ARDRFCNDDEIHRHGQEDLPRPSISAAEGTVIPLGSHVTFVCRGPVGVQTFRLEKDSRSIYNDTENVSQPSPSESEARFRIDSVSEGNAGLYRCVYYKAPKWSAQSDYLELLVKGQEVTWALFTSCGGDGEEPDYDMDV

SEQ ID NO: 123 - protein, 12 aa, Artificial Sequence
  MGC1 CDRL1 aa
QSVLYRSKNKNY

SEQ ID NO: 124 - skipped

SEQ ID NO: 125 - protein, 15 aa, Artificial Sequence
  MGC1 CDRL2 long aa
YYCLQYYITPYTFGQ

SEQ ID NO: 126 - protein, 9 aa, Artificial Sequence
  MGC1 CDRL3 aa
LQYYITPYT

SEQ ID NO: 127 - DNA, 24 bp, Artificial Sequence
  MGC1 CDRH1 nuc
gggttcaactttagaaagtcttgg

SEQ ID NO: 128 - DNA, 24 bp, Artificial Sequence
  MGC1 CDRH2 nuc
ataagagaagatggaagtgagagt

SEQ ID NO: 129 - DNA, 417 bp, Artificial Sequence
  MGC1 CDRH3 nuc
gcgagagatagattctgcaatgatgatgagattcacagacacggacaagaagatctgcccagaccctccatctcggctgccgaaggcaccgtgatccccctggggagccatgtgactttcgtgtgccggggcccggttggggttcaaacattccgcctggagaaggacagtagatccatatacaatgatactgaaaatgtgtctcaacctagtccatctgagtcagaggccagatttcgcattgactcagtgagtgaaggaaatgccggactttatcggtgcgtctattataaggcccctaaatggtctgcgcagagtgattacctggagctgctggtgaaaggtcaggaagtcacctgggccctgtttacctcctgtggtggtgatggagaggaacccgactacgacatggacgtc

SEQ ID NO: 130 - DNA, 36 bp, Artificial Sequence
  MGC1 CDRL1 nuc
cagagtgttttatacaggtccaagaataagaactac

SEQ ID NO: 131 - skipped

SEQ ID NO: 132 - DNA, 27 bp, Artificial Sequence
  MGC1 CDRL2 long nuc
ctcatttactcgacatctactcgggcg

SEQ ID NO: 133 - DNA, 27 bp, Artificial Sequence
  MGC1 CDRL3 nuc
ctgcaatattatattactccctacact

SEQ ID NO: 134 - protein, 246 aa, Artificial Sequence
  MGC1 heavy chain aa
EVQLVESGGGLVQPGGSLRLSCVASGFNFRKSWMGWVRQAPGKGLEWVANIREDGSESFYADSVKGRFTVSRDNAKKSLYLHINSLRAEDTAVYYCARDRFCNDDEIHRHGQEDLPRPSISAAEGTVIPLGSHVTFVCRGPVGVQTFRLEKDSRSIYNDTENVSQPSPSESEARFRIDSVSEGNAGLYRCVYYKAPKWSAQSDYLELLVKGQEVTWALFTSCGGDGEEPDYDMDVRGKGTTVTVSS

SEQ ID NO: 135 - protein, 113 aa, Artificial Sequence
  MGC1 light chain aa
DIVMTQSPDSLAVSLGERATINCKSSQSVLYRSKNKNYLAWFQQKPGQPPKVLIYSTSTRASGVPDRFTGSGSGTDFTLTISSLQAEDVAVYYCLQYYITPYTFGQGTKLEIK

SEQ ID NO: 136 - DNA, 738 bp, Artificial Sequence
  MGC1 heavy chain nuc
gaggtgcagctggtggagtctgggggaggcttggtccagccgggggggtccctgagactctcctgtgtagcctctgggttcaactttagaaagtcttggatgggttgggtccgccaggctccagggaaggggctggagtgggtggcaaacataagagaagatggaagtgagagtttctatgcggactctgtgaagggccgcttcaccgtctccagagacaacgccaagaaatcactgtatctccatatcaacagcctgagagccgaggacacggctgtctattactgtgcgagagatagattctgcaatgatgatgagattcacagacacggacaagaagatctgcccagaccctccatctcggctgccgaaggcaccgtgatccccctggggagccatgtgactttcgtgtgccggggcccggttggggttcaaacattccgcctggagaaggacagtagatccatatacaatgatactgaaaatgtgtctcaacctagtccatctgagtcagaggccagatttcgcattgactcagtgagtgaaggaaatgccggactttatcggtgcgtctattataaggcccctaaatggtctgcgcagagtgattacctggagctgctggtgaaaggtcaggaagtcacctgggccctgtttacctcctgtggtggtgatggagaggaacccgactacgacatggacgtccggggcaaagggaccacggtcaccgtctcctca

SEQ ID NO: 137 - DNA, 339 bp, Artificial Sequence
  MGC1 light chain nuc
gacatcgtgatgacccagtctccagactccctggctgtgtctctgggcgagagggccaccatcaactgcaagtccagtcagagtgttttatacaggtccaagaataagaactacttagcttggttccagcagaaaccaggacagcctcctaaggtgctcatttactcgacatctactcgggcgtccggggtccctgaccgattcactggcagcgggtctgggacagatttcactctcaccatcagcagcctgcaggctgaagatgtggcagtttattactgtctgcaatattatattactccctacacttttggccaggggaccaagttggagatcaaa

SEQ ID NO: 138 - protein, 8 aa, Artificial Sequence
  MGC2 CDRH1 aa
GFTFSNFW

SEQ ID NO: 139 - protein, 8 aa, Artificial Sequence
  MGC2 CDRH2 aa
IKEDGSEK

SEQ ID NO: 140 - protein, 136 aa, Artificial Sequence
  MGC2 CDRH3 aa
VRERFCSNHIHKEEHLPRPSISPEPGTVITLGSHVTFVCRGPVGVQTFRLEKDSRSTYNDTEDVSQPSPSESEARFRIDSVSEGYAGLYRCLYYKPPKWSEQSDYLELLVKGDDVTWALYPSCGGDGEASDYNMDV

SEQ ID NO: 141 - protein, 6 aa, Artificial Sequence
  MGC2 CDRL1 aa
QRFSGW

SEQ ID NO: 142 - skipped

SEQ ID NO: 143 - protein, 9 aa, Artificial Sequence
  MGC2 CDRL2 long aa
LIYKASPLA

SEQ ID NO: 144 - protein, 9 aa, Artificial Sequence
  MGC2 CDRL3 aa
QHYSNYSYT

SEQ ID NO: 145 - DNA, 24 bp, Artificial Sequence
  MGC2 CDRH1 nuc
ggattcacctttagtaacttttgg

SEQ ID NO: 146 - DNA, 24 bp, Artificial Sequence
  MGC2 CDRH2 nuc
ataaaggaagatggaagtgagaaa

SEQ ID NO: 147 - DNA, 408 bp, Artificial Sequence
  MGC2 CDRH3 nuc
gtgagagagagattctgcagtaatcatatccacaaagaagagcatctgcccagaccctccatctcgcctgagccaggcaccgtgatcaccctggggagccatgtgactttcgtgtgccggggcccggttggggttcaaacattccgcctggagaaggacagtagatccacatacaatgatactgaagatgtgtctcaacctagtccatctgagtcagaggccagattccgcattgactcagtaagtgaaggatatgccgggctttatcgctgcctctattataagccccctaaatggtctgagcagagtgactacctggagctgctggtgaaaggtgacgacgtcacctgggccctgtacccctcttgtggtggtgatggagaggcttccgactacaacatggacgtc

SEQ ID NO: 148 - DNA, 18 bp, Artificial Sequence
  MGC2 CDRL1 nuc
cagcgttttagtggctgg

SEQ ID NO: 149 - skipped

SEQ ID NO: 150 - DNA, 27 bp, Artificial Sequence
  MGC2 CDRL2 long nuc
ctgatctataaggcgtctcctttagca

SEQ ID NO: 151 - DNA, 27 bp, Artificial Sequence
  MGC2 CDRL3 nuc
caacactacagtaattattcatatact

SEQ ID NO: 152 - protein, 243 aa, Artificial Sequence
  MGC2 heavy chain aa
EVQLVESGGGLVQPGGSLRLSCAASGFTFSNFWMGWVRQTPGKGLEWVANIKEDGSEKYYVDSVRGRFTISRDSAKNSLYLQMNSLRAEDTAVYYCVRERFCSNHIHKEEHLPRPSISPEPGTVITLGSHVTFVCRGPVGVQTFRLEKDSRSTYNDTEDVSQPSPSESEARFRIDSVSEGYAGLYRCLYYKPPKWSEQSDYLELLVKGDDVTWALYPSCGGDGEASDYNMDVWGKGTTVTVSS

SEQ ID NO: 153 - protein, 107 aa, Artificial Sequence
  MGC2 light chain aa
DIQMTQSPSTLSASVGDRVTISCRASQRFSGWLAWYQQKPGKAPNLLIYKASPLAGGGPSRFSGSGSGTDFTLTISSLQPDDSATYYCQHYSNYSYTFGQGTKLEIR

SEQ ID NO: 154 - DNA, 729 bp, Artificial Sequence
  MGC2 heavy chain nuc
gaggtgcagctggtggagtctgggggaggcttggtccagcctggggggtccctgagactctcctgtgcagcctctggattcacctttagtaacttttggatgggttgggtccgccagactccagggaaggggctggagtgggtggccaatataaaggaagatggaagtgagaaatactatgtggactctgtgaggggccgattcaccatctccagagacagcgccaagaactcactttatctgcagatgaacagcctgagagccgaggacacggctgtctattattgtgtgagagagagattctgcagtaatcatatccacaaagaagagcatctgcccagaccctccatctcgcctgagccaggcaccgtgatcaccctggggagccatgtgactttcgtgtgccggggcccggttggggttcaaacattccgcctggagaaggacagtagatccacatacaatgatactgaagatgtgtctcaacctagtccatctgagtcagaggccagattccgcattgactcagtaagtgaaggatatgccgggctttatcgctgcctctattataagccccctaaatggtctgagcagagtgactacctggagctgctggtgaaaggtgacgacgtcacctgggccctgtacccctcttgtggtggtgatggagaggcttccgactacaacatggacgtctggggcaaagggaccacggtcaccgtctcctca

SEQ ID NO: 155 - DNA, 321 bp, Artificial Sequence
  MGC2 light chain nuc
gacatccagatgacccagtctccttccaccctgtctgcatctgtgggagacagagtcaccatctcttgccgggccagtcagcgttttagtggctggttggcctggtatcagcagaaaccagggaaagcccctaacctcctgatctataaggcgtctcctttagcaggtgggggcccatcaaggttcagcggcagtggatctgggacagacttcactctcaccatcagcagcctgcagcctgatgattctgcaacttattactgccaacactacagtaattattcatatacttttggccaggggaccaagctggagatcaga

SEQ ID NO: 156 - protein, 8 aa, Artificial Sequence
  MGC4 CDRH1 aa
GFNSRSYW

SEQ ID NO: 157 - protein, 8 aa, Artificial Sequence
  MGC4 CDRH2 aa
INQDGTEK

SEQ ID NO: 158 - protein, 136 aa, Artificial Sequence
  MGC4 CDRH3 aa
ARDRFCGGESHLHGEEDLPRPSISAEPDTVIPLGSHVTFVCRGPVGVHTFRLERGWRYNDTEDVSQAGPSESEARFRIDSVREGNAGLYRCIYYIAPKWSEQSDYLELRVKGGDVTWALLTYCGGDGEESDYPMDV

SEQ ID NO: 159 - protein, 9 aa, Artificial Sequence
  MGC4 CDRL1 aa
TGPVTSAYY

SEQ ID NO: 160 - skipped

SEQ ID NO: 161 - protein, 9 aa, Artificial Sequence
  MGC4 CDRL2 long aa
LIYSINKKH

SEQ ID NO: 162 - protein, 11 aa, Artificial Sequence
  MGC4 CDRL3 aa
LLSCGGAQPWV

SEQ ID NO: 163 - DNA, 24 bp, Artificial Sequence
  MGC4 CDRH1 nuc
ggattcaactctcgtagttattgg

SEQ ID NO: 164 - DNA, 24 bp, Artificial Sequence
  MGC4 CDRH2 nuc
ataaatcaagatgggactgagaaa

SEQ ID NO: 165 - DNA, 408 bp, Artificial Sequence
  MGC4 CDRH3 nuc
gcgagagacagattctgtggtggtgagagtcacttgcacggagaagaagatctgcccagaccctccatctcggctgagccagacaccgtaatccccctggggagccatgtgactttcgtgtgccggggcccggttggggttcacacattccgcctggagagggggtggaggtacaacgacactgaagatgtgtctcaagctggtccatctgagtcagaggccagattccgcattgactcggtaagggaaggaaatgccgggctttatcgatgcatctattacatagcccctaaatggtctgagcagagtgactacctggagctgcgggtgaaaggtggggacgtcacctgggccctgttaacgtactgtggcggtgatggagaggaatccgactaccccatggacgtc

SEQ ID NO: 166 - DNA, 27 bp, Artificial Sequence
  MGC4 CDRL1 nuc
actggacctgtcaccagtgcttactat

SEQ ID NO: 167 - skipped

SEQ ID NO: 168 - DNA, 27 bp, Artificial Sequence
  MGC4 CDRL2 long nuc
cttatttatagtataaacaaaaaacac

SEQ ID NO: 169 - DNA, 33 bp, Artificial Sequence
  MGC4 CDRL3 nuc
ctgctctcctgtggtggtgctcagccttgggtg

SEQ ID NO: 170 - protein, 243 aa, Artificial Sequence
  MGC4 heavy chain aa
EVQLVESGGGLVQPGGSLRLSCEGSGFNSRSYWMTWVRQAPGKGLEWVASINQDGTEKNYVDSVKGRFTISRDSAKNSLYLQMSSLRADDTAVYYCARDRFCGGESHLHGEEDLPRPSISAEPDTVIPLGSHVTFVCRGPVGVHTFRLERGWRYNDTEDVSQAGPSESEARFRIDSVREGNAGLYRCIYYIAPKWSEQSDYLELRVKGGDVTWALLTYCGGDGEESDYPMDVWGKGTTVTVSS

SEQ ID NO: 171 - protein, 111 aa, Artificial Sequence
  MGC4 light chain aa
QTVVTQEPSLTVSPGGTVTLTCASSTGPVTSAYYPNWFQQKPGQAPRSLIYSINKKHSWTPARFSGSLLGGKAALTLSGVQPEDEADYYCLLSCGGAQPWVFGGGTKLTVQ

SEQ ID NO: 172 - DNA, 729 bp, Artificial Sequence
  MGC4 heavy chain nuc
gaggtgcagctggtggagtctgggggaggcttggtacagcctggggggtccctgagactctcctgtgaaggctctggattcaactctcgtagttattggatgacctgggtccgccaggctccagggaaggggctggagtgggtggccagtataaatcaagatgggactgagaaaaattatgtggactctgtgaagggccggttcaccatctccagagactccgccaagaactcactgtatctgcaaatgagcagcctgagagccgacgacacggctgtatattactgtgcgagagacagattctgtggtggtgagagtcacttgcacggagaagaagatctgcccagaccctccatctcggctgagccagacaccgtaatccccctggggagccatgtgactttcgtgtgccggggcccggttggggttcacacattccgcctggagagggggtggaggtacaacgacactgaagatgtgtctcaagctggtccatctgagtcagaggccagattccgcattgactcggtaagggaaggaaatgccgggctttatcgatgcatctattacatagcccctaaatggtctgagcagagtgactacctggagctgcgggtgaaaggtggggacgtcacctgggccctgttaacgtactgtggcggtgatggagaggaatccgactaccccatggacgtctggggcaaagggaccacggtcaccgtctcctca

SEQ ID NO: 173 - DNA, 334 bp, Artificial Sequence
  MGC4 light chain nuc
cagactgtggttactcaggagccctcactgactgtgtccccaggagggacagtcactctcacctgtgcttccagcactggacctgtcaccagtgcttactatccaaactggttccagcagaagcctggacaagcacccaggtctcttatttatagtataaacaaaaaacactcctggacccctgcccggttctcaggctccctccttgggggcaaagctgccctgacactgtcaggtgtacagcctgaggacgaggctgactattactgcctgctctcctgtggtggtgctcagccttgggtgttcggcggagggaccaagctgaccgtccaag

SEQ ID NO: 174 - protein, 8 aa, Artificial Sequence
  MGC5 CDRH1 aa
GFNSRSYW

SEQ ID NO: 175 - protein, 8 aa, Artificial Sequence
  MGC5 CDRH2 aa
INQDGTEK

SEQ ID NO: 176 - protein, 136 aa, Artificial Sequence
  MGC5 CDRH3 aa
ARDRFCGGESHLHGEEDLPRPSISAEPGTVIPLGSHVTFVCRGPVGVHTFRLERGWRYNDTEDVSQAGPSQSEARFRIDSVREGNAGLYRCLYYIPPKWSEQSDYLELRVKGGDVTWALLTYCGGDGEESDYPMDV

SEQ ID NO: 177 - protein, 9 aa, Artificial Sequence
  MGC5 CDRL1 aa
TGPVTSAYY

SEQ ID NO: 178 - skipped

SEQ ID NO: 179 - protein, 9 aa, Artificial Sequence
  MGC5 CDRL2 long aa
LIYNINKKH

SEQ ID NO: 180 - protein, 11 aa, Artificial Sequence
  MGC5 CDRL3 aa
LLSCGGAQPWV

SEQ ID NO: 181 - DNA, 24 bp, Artificial Sequence
  MGC5 CDRH1 nuc
ggattcaactctcgtagttattgg

SEQ ID NO: 182 - DNA, 24 bp, Artificial Sequence
  MGC5 CDRH2 nuc
ataaatcaagatggaactgagaaa

SEQ ID NO: 183 - DNA, 408 bp, Artificial Sequence
  MGC5 CDRH3 nuc
gcgagagacagattctgtggtggtgagagtcacttgcacggagaagaagatctgcccagaccctccatctcggctgagccaggcaccgtgatccccctggggagccatgtgactttcgtgtgccggggccccgttggggttcacacattccgcctggagagggggtggagatacaacgacactgaagatgtgtctcaagctggtccatctcagtcagaggccagattccgcattgactcggtaagggaaggaaatgccgggctttatcgatgcctctattacataccccctaaatggtctgagcagagtgactacctggaactgcgggtgaaaggtggggacgtcacctgggccctgttaacgtactgtggtggtgatggagaggaatccgactaccccatggacgtc

SEQ ID NO: 184 - DNA, 27 bp, Artificial Sequence
  MGC5 CDRL1 nuc
actggacctgtcaccagtgcttactat

SEQ ID NO: 185 - skipped

SEQ ID NO: 186 - DNA, 27 bp, Artificial Sequence
  MGC5 CDRL2 long nuc
cttatttataatataaacaaaaaacac

SEQ ID NO: 187 - DNA, 33 bp, Artificial Sequence; identical to SEQ ID NO: 169
  MGC5 CDRL3 nuc

SEQ ID NO: 188 - protein, 243 aa, Artificial Sequence
  MGC5 heavy chain aa
EVQLVESGGGLVQPGGSLRLSCEASGFNSRSYWMTWVRQAPGKGLEWVATINQDGTEKNYVDSVRGRFTISRDTAKNSLFLQMNSLRAEDTAVYYCARDRFCGGESHLHGEEDLPRPSISAEPGTVIPLGSHVTFVCRGPVGVHTFRLERGWRYNDTEDVSQAGPSQSEARFRIDSVREGNAGLYRCLYYIPPKWSEQSDYLELRVKGGDVTWALLTYCGGDGEESDYPMDVWGKGTTVTVSS

SEQ ID NO: 189 - protein, 111 aa, Artificial Sequence
  MGC5 light chain aa
QTVVTQEPSLTVSPGGTVTLTCASNTGPVTSAYYPNWFQQKPGQAPRSLIYNINKKHSWTPARFSGSLLGGKAALTLSGVQPEDEADYYCLLSCGGAQPWVFGGGTKLTVQ

SEQ ID NO: 190 - DNA, 729 bp, Artificial Sequence
  MGC5 heavy chain nuc
gaggtgcagctggtggagtctgggggaggcttggtccagcctggggggtcactgagactctcctgtgaagcctctggattcaactctcgtagttattggatgacctgggtccgccaggctccagggaaggggctggagtgggtggccactataaatcaagatggaactgagaaaaattatgtggactctgtgaggggccggttcaccatctccagagacaccgccaagaactcactgtttctgcaaatgaacagcctgagagccgaggacacggctgtatattactgcgcgagagacagattctgtggtggtgagagtcacttgcacggagaagaagatctgcccagaccctccatctcggctgagccaggcaccgtgatccccctggggagccatgtgactttcgtgtgccggggccccgttggggttcacacattccgcctggagagggggtggagatacaacgacactgaagatgtgtctcaagctggtccatctcagtcagaggccagattccgcattgactcggtaagggaaggaaatgccgggctttatcgatgcctctattacataccccctaaatggtctgagcagagtgactacctggaactgcgggtgaaaggtggggacgtcacctgggccctgttaacgtactgtggtggtgatggagaggaatccgactaccccatggacgtctggggcaaagggaccacggtcaccgtctcctca

SEQ ID NO: 191 - DNA, 333 bp, Artificial Sequence
  MGC5 light chain nuc
cagactgtggtgactcaggagccctcactgactgtgtccccaggagggacagtcactctcacctgtgcttccaacactggacctgtcaccagtgcttactatccaaactggttccagcagaagcctggacaagcacccaggtctcttatttataatataaacaaaaaacactcctggacccctgcccggttctcaggctccctccttgggggcaaagctgccctgacactgtcaggtgtacagcctgaggacgaggctgactattactgcctgctctcctgtggtggtgctcagccttgggtgttcggcggagggaccaagctgaccgtccaa

SEQ ID NO: 192 - protein, 8 aa, Artificial Sequence
  MGC7 CDRH1 aa
GFTFRNYW

SEQ ID NO: 193 - protein, 8 aa, Artificial Sequence
  MGC7 CDRH2 aa
IRQDGSEK

SEQ ID NO: 194 - protein, 138 aa, Artificial Sequence
  MGC7 CDRH3 aa
VRDKFCSDENHMHVADDLPRPSISPEPGTVIPLGSHVTFVCRGPVGVQTFRLEKDRRSTYNDTEDVSQPSPSESEARFRIDSVTEGNAGLYRCVYYKPPKWSDQSDFLELLVKGEDVTWALFPHCGADGEDSDYYMDV

SEQ ID NO: 195 - protein, 6 aa, Artificial Sequence
  MGC7 CDRL1 aa
QGLSTW

SEQ ID NO: 196 - skipped

SEQ ID NO: 197 - protein, 9 aa, Artificial Sequence
  MGC7 CDRL2 long aa
LIYAASSLQ

SEQ ID NO: 198 - protein, 9 aa, Artificial Sequence
  MGC7 CDRL3 aa
QQANSFPLT

SEQ ID NO: 199 - DNA, 24 bp, Artificial Sequence
  MGC7 CDRH1 nuc
ggattcaccttcagaaattattgg

SEQ ID NO: 200 - DNA, 24 bp, Artificial Sequence
  MGC7 CDRH2 nuc
ataaggcaagatggaagtgagaag

SEQ ID NO: 201 - DNA, 414 bp, Artificial Sequence
  MGC7 CDRH3 nuc
gtgagagataaattctgcagtgatgagaatcacatgcacgtagcagatgatctgcccagaccctctatctcgcctgagccaggcaccgtgatccccctggggagccatgtgactttcgtgtgtcggggcccggttggggttcaaacattccgcctggagaaggacagaagatccacatacaatgatactgaagatgtgtctcaacctagtccatctgagtcagaggccagattccgcattgactcagtaactgaaggaaatgccgggctttatcgctgcgtctattataagccccctaaatggtctgaccagagtgacttcctggagttgctggtgaagggtgaggacgtcacctgggccctgttcccccattgtggtgctgatggagaggactccgactactacatggacgtc

SEQ ID NO: 202 - DNA, 18 bp, Artificial Sequence
  MGC7 CDRL1 nuc
cagggtcttagtacctgg

SEQ ID NO: 203 - skipped

SEQ ID NO: 204 - DNA, 45 bp, Artificial Sequence
  MGC7 CDRL2 long nuc
tattattgtcaacaggctaacagtttccctctcactttcggcgga

SEQ ID NO: 205 - DNA, 27 bp, Artificial Sequence
  MGC7 CDRL3 nuc
caacaggctaacagtttccctctcact

SEQ ID NO: 206 - protein, 245 aa, Artificial Sequence
  MGC7 heavy chain aa
EVQLVESGGDLVQPGGSLRLSCAASGFTFRNYWMSWVRQTPGKGLEWVANIRQDGSEKYYVDSVKGRFTISRDNAKNLLYLQMNSLRAEDTAVYYCVRDKFCSDENHMHVADDLPRPSISPEPGTVIPLGSHVTFVCRGPVGVQTFRLEKDRRSTYNDTEDVSQPSPSESEARFRIDSVTEGNAGLYRCVYYKPPKWSDQSDFLELLVKGEDVTWALFPHCGADGEDSDYYMDVWGKGTTVTVSS

SEQ ID NO: 207 - protein, 107 aa, Artificial Sequence
  MGC7 light chain aa
DIQMTQSPSSVSASVGDRVTITCRASQGLSTWLAWYQQKPGKAPKILIYAASSLQSGVPSRFSGSGSGTDFTLTISSLQPEDFATYYCQQANSFPLTFGGGTKVEIK

SEQ ID NO: 208 - DNA, 735 bp, Artificial Sequence
  MGC7 heavy chain nuc
gaggtgcagctggtggagtctgggggagacttggtccagcctggggggtccctgagactctcctgtgcagcctctggattcaccttcagaaattattggatgagttgggtccgccagactccagggaagggactggagtgggtggccaacataaggcaagatggaagtgagaagtattatgtggactctgtgaagggccgattcaccatctccagagacaacgccaagaacttattatatctacaaatgaacagcctgagagccgaggacacggctgtgtattactgtgtgagagataaattctgcagtgatgagaatcacatgcacgtagcagatgatctgcccagaccctctatctcgcctgagccaggcaccgtgatccccctggggagccatgtgactttcgtgtgtcggggcccggttggggttcaaacattccgcctggagaaggacagaagatccacatacaatgatactgaagatgtgtctcaacctagtccatctgagtcagaggccagattccgcattgactcagtaactgaaggaaatgccgggctttatcgctgcgtctattataagccccctaaatggtctgaccagagtgacttcctggagttgctggtgaagggtgaggacgtcacctgggccctgttcccccattgtggtgctgatggagaggactccgactactacatggacgtctggggcaaagggaccacggtcaccgtctcctca

SEQ ID NO: 209 - DNA, 321 bp, Artificial Sequence
  MGC7 light chain nuc
gacatccagatgacccagtctccatcttccgtgtctgcatctgtaggagacagagtcaccatcacttgtcgggcgagtcagggtcttagtacctggttagcctggtatcagcagaaaccagggaaagcccctaagatcctgatctatgctgcatccagtttgcaaagtggggtcccatcaaggttcagcggcagtggatctgggacagatttcactctcaccatcagcagcctgcagcctgaagattttgcaacttattattgtcaacaggctaacagtttccctctcactttcggcggagggaccaaggtggagatcaaa

SEQ ID NO: 210 - protein, 8 aa, Artificial Sequence
  MGC17 CDRH1 aa
GFNFRKSW

SEQ ID NO: 211 - protein, 8 aa, Artificial Sequence
  MGC17 CDRH2 aa
IREDGSKA

SEQ ID NO: 212 - protein, 139 aa, Artificial Sequence
  MGC17 CDRH3 aa
ARDRFCSDDEDHSHGAEDLPRPSISAEEGTVIPLGSRLTFVCRGPVGVHTFRLERDRRSTYNDTEDVSHPSPSESEARFRIDSVSEGNAGLYRCVYYKSPEWSKQSDYLELLVKGQEVTWALFTSCGGDGEVPDYDMDV

SEQ ID NO: 213 - protein, 12 aa, Artificial Sequence
  MGC17 CDRL1 aa
QSVLYRSKNKKY

SEQ ID NO: 214 - skipped

SEQ ID NO: 215 - protein, 9 aa, Artificial Sequence
  MGC17 CDRL2 long aa
LIYWTSTRA

SEQ ID NO: 216 - protein, 9 aa, Artificial Sequence
  MGC17 CDRL3 aa
QQYFIFPYT

SEQ ID NO: 217 - DNA, 24 bp, Artificial Sequence
  MGC17 CDRH1 nuc
gggttcaattttagaaagtcttgg

SEQ ID NO: 218 - DNA, 24 bp, Artificial Sequence
  MGC17 CDRH2 nuc
ataagagaagatggaagtaaggca

SEQ ID NO: 219 - DNA, 417 bp, Artificial Sequence
  MGC17 CDRH3 nuc
gcgagagatagattctgcagtgatgatgaggatcacagccacggagcagaagatctgcccagaccctccatctcggctgaggaaggcaccgtgattcccctggggagccgtctgactttcgtgtgccggggcccggttggggttcacacattccgcctggagagggaccgtagatccacatacaatgatactgaagatgtgtctcaccctagtccatctgagtctgaggccagatttcgcattgactcagtgagtgaaggaaatgccgggctttatcgctgcgtctattataagtcccctgaatggtctaagcagagtgattacctggagctgctggtgaaaggtcaggaagtcacctgggccctgtttacttcttgtggtggtgatggagaggtacccgactacgacatggacgtc

SEQ ID NO: 220 - DNA, 36 bp, Artificial Sequence
  MGC17 CDRL1 nuc
cagagtgttttatacaggtccaagaataagaaatat

SEQ ID NO: 221 - skipped

SEQ ID NO: 222 - DNA, 27 bp, Artificial Sequence
  MGC17 CDRL2 long nuc
ctcatttactggacatctactcgggcg

SEQ ID NO: 223 - DNA, 27 bp, Artificial Sequence
  MGC17 CDRL3 nuc
cagcagtattttatttttccgtacact

SEQ ID NO: 224 - protein, 246 aa, Artificial Sequence
  MGC17 heavy chain aa
EVQLVESGGGLVQPGGSLKLSCVASGFNFRKSWMSWVRQAPGKGLEWVANIREDGSKAYYVDSVKGRFTVSRDNAKNSLYLQINSLRADDTAVYYCARDRFCSDDEDHSHGAEDLPRPSISAEEGTVIPLGSRLTFVCRGPVGVHTFRLERDRRSTYNDTEDVSHPSPSESEARFRIDSVSEGNAGLYRCVYYKSPEWSKQSDYLELLVKGQEVTWALFTSCGGDGEVPDYDMDVRGKGTTVTVSS

SEQ ID NO: 225 - protein, 113 aa, Artificial Sequence
  MGC17 light chain aa
DIVMTQSPDSLAVSLGERATINCKSSQSVLYRSKNKKYLAWFQQRPGQPPKVLIYWTSTRASGVPDRFSGSGSGTDFTLTISSLQADDVAVYYCQQYFIFPYTFGQGTKLEIR

SEQ ID NO: 226 - DNA, 738 bp, Artificial Sequence
  MGC17 heavy chain nuc
gaggtgcagctggtggagtcggggggaggcttggtccagcctggggggtccctgaaactgtcctgtgtagcctctgggttcaattttagaaagtcttggatgagttgggtccgccaggctccagggaaggggctggagtgggtggcaaacataagagaagatggaagtaaggcatactatgtggactctgtcaagggccgattcaccgtctccagagacaacgccaagaactcgctgtatctgcagatcaacagcctgagagccgacgacacggctgtctattactgtgcgagagatagattctgcagtgatgatgaggatcacagccacggagcagaagatctgcccagaccctccatctcggctgaggaaggcaccgtgattcccctggggagccgtctgactttcgtgtgccggggcccggttggggttcacacattccgcctggagagggaccgtagatccacatacaatgatactgaagatgtgtctcaccctagtccatctgagtctgaggccagatttcgcattgactcagtgagtgaaggaaatgccgggctttatcgctgcgtctattataagtcccctgaatggtctaagcagagtgattacctggagctgctggtgaaaggtcaggaagtcacctgggccctgtttacttcttgtggtggtgatggagaggtacccgactacgacatggacgtccggggcaaagggaccacggtcaccgtctcttca

SEQ ID NO: 227 - DNA, 339 bp, Artificial Sequence
  MGC17 light chain nuc
gacatcgtgatgacccaatctcctgactccctggctgtgtctctgggcgagagggccaccatcaactgcaagtccagccagagtgttttatacaggtccaagaataagaaatatttagcttggttccagcagagaccaggacagcctcctaaggttctcatttactggacatctactcgggcgtccggggtccctgaccgattcagtggcagcgggtctgggacagatttcactctcaccatcagcagcctgcaggctgatgatgtggcagtttattattgtcagcagtattttatttttccgtacacttttggccaggggaccaagttggagatcaga

SEQ ID NO: 228 - protein, 8 aa, Artificial Sequence
  MGC26 CDRH1 aa
GFTFSTYW

SEQ ID NO: 229 - protein, 8 aa, Artificial Sequence
  MGC26 CDRH2 aa
IKQDGTER

SEQ ID NO: 230 - protein, 136 aa, Artificial Sequence
  MGC26 CDRH3 aa
VRDRFCRDHMHIEEDLPRPSISPEPATVIPLGSHVTIVCRGPVGVETFRLQKESRSLYNDTEDVSQPSPSESEARFRIDSVSEGHGGLYRCLYYKSSKWSEQSDYLEMLVKGEDVTWALFPYCGGDGEESDYYMDV

SEQ ID NO: 231 - protein, 6 aa, Artificial Sequence
  MGC26 CDRL1 aa
QRLSRS

SEQ ID NO: 232 - skipped

SEQ ID NO: 233 - protein, 9 aa, Artificial Sequence
  MGC26 CDRL2 long aa
LIYKASPLE

SEQ ID NO: 234 - protein, 9 aa, Artificial Sequence
  MGC26 CDRL3 aa
QQYSNYSYS

SEQ ID NO: 235 - DNA, 24 bp, Artificial Sequence
  MGC26 CDRH1 nuc
ggattcacctttagtacttattgg

SEQ ID NO: 236 - protein, 24 aa, Artificial Sequence
  MGC26 CDRH2 nuc
ATAAAGCAAGATGGAACTGAGAGA

SEQ ID NO: 237 - DNA, 408 bp, Artificial Sequence
  MGC26 CDRH3 nuc
gtgagagacagattctgcagagatcacatgcacatagaagaagatctgcccagaccctccatctcgccggagccagccaccgtgatccccctggggagccatgtgactatcgtgtgccggggcccggttggggttgaaacattccgcctgcagaaggagagtagatccctgtacaatgacactgaagatgtgtctcaacctagtccatctgagtcagaggccagattccgcattgactcagtaagtgaagggcatggcgggctttatcgctgcctctattataagtcttctaaatggtctgagcagagtgactacctggagatgctggtgaaaggtgaggacgtcacctgggccctgttcccctattgtggtggtgatggagaggaatccgactactacatggacgtc

SEQ ID NO: 238 - DNA, 18 bp, Artificial Sequence
  MGC26 CDRL1 nuc
cagcgtcttagtcgctcg

SEQ ID NO: 239 - skipped

SEQ ID NO: 240 - DNA, 27 bp, Artificial Sequence
  MGC26 CDRL2 long nuc
ctgatctataaggcgtctcctttagaa

SEQ ID NO: 241 - DNA, 27 bp, Artificial Sequence
  MGC26 CDRL3 nuc
caacaatacagtaattattcatatagt

SEQ ID NO: 242 - protein, 243 aa, Artificial Sequence
  MGC26 heavy chain aa
EVQLVDSGGGLVQPGGSLRLSCAASGFTFSTYWMTWVRQTPGKGLEWVASIKQDGTERYYVDSVKGRFIISRDNAKNSLYLQMHSLRAEDTAVYYCVRDRFCRDHMHIEEDLPRPSISPEPATVIPLGSHVTIVCRGPVGVETFRLQKESRSLYNDTEDVSQPSPSESEARFRIDSVSEGHGGLYRCLYYKSSKWSEQSDYLEMLVKGEDVTWALFPYCGGDGEESDYYMDVWGKGTTVTVSS

SEQ ID NO: 243 - protein, 107 aa, Artificial Sequence
  MGC26 light chain aa
DIQLTQSPSTLSASVGDRVTISCRASQRLSRSLAWYQQRPRKAPNLLIYKASPLEIGGPSRFTGSGSGTEFTLTISSLQPDDSATYYCQQYSNYSYSFGQGTKLEIR

SEQ ID NO: 244 - DNA, 729 bp, Artificial Sequence
  MGC26 heavy chain nuc
gaggtgcagctggtggattctgggggaggcttggtccagcctggggggtccctgagactctcctgtgcagcctctggattcacctttagtacttattggatgacctgggtccgccagactccagggaaggggctggagtgggtggccagcataaagcaagatggaactgagagatactatgtggactctgtgaagggccgattcattatctccagagacaacgccaagaactcactatatttgcaaatgcacagcctgagagccgaggacacggctgtgtattattgtgtgagagacagattctgcagagatcacatgcacatagaagaagatctgcccagaccctccatctcgccggagccagccaccgtgatccccctggggagccatgtgactatcgtgtgccggggcccggttggggttgaaacattccgcctgcagaaggagagtagatccctgtacaatgacactgaagatgtgtctcaacctagtccatctgagtcagaggccagattccgcattgactcagtaagtgaagggcatggcgggctttatcgctgcctctattataagtcttctaaatggtctgagcagagtgactacctggagatgctggtgaaaggtgaggacgtcacctgggccctgttcccctattgtggtggtgatggagaggaatccgactactacatggacgtctggggcaaagggaccacggtcaccgtctcctca

SEQ ID NO: 245 - DNA, 321 bp, Artificial Sequence
  MGC26 light chain nuc
gacatccagctgacccagtctccttccaccctgtctgcatctgtaggagacagagtcaccatctcttgccgggccagtcagcgtcttagtcgctcgttggcctggtatcagcagagaccacggaaagcccctaacctcctgatctataaggcgtctcctttagaaattgggggcccatcaaggttcaccggcagtggatctgggacagaattcactctcaccatcagcagcctgcagcctgatgattctgcaacttattactgccaacaatacagtaattattcatatagttttggccaggggaccaagctggagatcaga

SEQ ID NO: 246 - protein, 8 aa, Artificial Sequence
  MGC28 CDRH1 aa
GFTFSSYW

SEQ ID NO: 247 - protein, 8 aa, Artificial Sequence
  MGC28 CDRH2 aa
INQDGSER

SEQ ID NO: 248 - protein, 137 aa, Artificial Sequence
  MGC28 CDRH3 aa
ARQRFCSDGSLFHGEDLPRPTISAETGTVISLGSHVTFVCRGPLGVQTFRLERESRSRYSETEDVSQVGPSESEARFRIDSVSEGNAGLYRCIYYKPPKWSEQSDYLELRVKGEDVTWALLTYCGGDRDESDYYMDV

SEQ ID NO: 249 - protein, 9 aa, Artificial Sequence
  MGC28 CDRL1 aa
TGSVTSGSF

SEQ ID NO: 250 - skipped

SEQ ID NO: 251 - protein, 9 aa, Artificial Sequence
  MGC28 CDRL2 long aa
LIYSTTKKH

SEQ ID NO: 252 - protein, 11 aa, Artificial Sequence
  MGC28 CDRL3 aa
LLYCGGGQPWV

SEQ ID NO: 253 - DNA, 24 bp, Artificial Sequence
  MGC28 CDRH1 nuc
ggattcacgtttagttcttattgg

SEQ ID NO: 254 - DNA, 24 bp, Artificial Sequence
  MGC28 CDRH2 nuc
ataaaccaagatggaagtgagaga

SEQ ID NO: 255 - DNA, 411 bp, Artificial Sequence
  MGC28 CDRH3 nuc
gcgagacaaagattctgcagtgatgggagtctctttcacggagaagatctgcccagacccaccatctcggctgagacaggcaccgtgatctccctggggagccatgtgactttcgtgtgccggggcccacttggggtgcaaacattccgcctggagagggagagtaggtccagatacagtgaaactgaagatgtgtctcaagttggtccatctgagtcagaggccagattccgcattgactcagtgagtgaaggaaatgccgggctttatcgatgcatctattacaaaccccctaaatggtctgagcagagtgactacctggagctgcgggtgaaaggtgaggacgtcacctgggccctgttaacctattgtggtggtgatagagacgaatccgactactacatggacgtc

SEQ ID NO: 256 - DNA, 27 bp, Artificial Sequence
  MGC28 CDRL1 nuc
actggatcagtcaccagtggttccttt

SEQ ID NO: 257 - skipped

SEQ ID NO: 258 - DNA, 27 bp, Artificial Sequence
  MGC28 CDRL2 long nuc
ctgatttatagtacaaccaaaaaacac

SEQ ID NO: 259 - DNA, 33 bp, Artificial Sequence
  MGC28 CDRL3 nuc
ctactctactgtggtggtggtcaaccttgggtg

SEQ ID NO: 260 - protein, 244 aa, Artificial Sequence
  MGC28 heavy chain aa
EVQLVESGGGLVQPGGSLRLSCEASGFTFSSYWMTWVRQAPGKGLEWVANINQDGSERYYVDSVKGRFTISRDTVKNSLYLQMNNLRAEDTAVYYCARQRFCSDGSLFHGEDLPRPTISAETGTVISLGSHVTFVCRGPLGVQTFRLERESRSRYSETEDVSQVGPSESEARFRIDSVSEGNAGLYRCIYYKPPKWSEQSDYLELRVKGEDVTWALLTYCGGDRDESDYYMDVWGKGTTVTVSS

SEQ ID NO: 261 - protein, 111 aa, Artificial Sequence
  MGC28 light chain aa
QTVVTQEPSLTVSPGGTVTLTCASSTGSVTSGSFPNWFQQTPGQAPRSLIYSTTKKHSWTPARFSGSLLGGKAALTVSDTQPEDEAEYYCLLYCGGGQPWVFGGGTKLTVL

SEQ ID NO: 262 - DNA, 732 bp, Artificial Sequence
  MGC28 heavy chain nuc
gaggtgcagctggtggagtctgggggaggcttggtccagccgggggggtccctgagactctcctgtgaagcctctggattcacgtttagttcttattggatgacctgggtccgccaggctccagggaaggggctggagtgggtggccaatataaaccaagatggaagtgagagatattatgtggactctgtgaagggccggttcaccatctccagagacaccgtcaagaactcactgtatttgcaaatgaacaacctgagagccgaggacacggctgtatattactgcgcgagacaaagattctgcagtgatgggagtctctttcacggagaagatctgcccagacccaccatctcggctgagacaggcaccgtgatctccctggggagccatgtgactttcgtgtgccggggcccacttggggtgcaaacattccgcctggagagggagagtaggtccagatacagtgaaactgaagatgtgtctcaagttggtccatctgagtcagaggccagattccgcattgactcagtgagtgaaggaaatgccgggctttatcgatgcatctattacaaaccccctaaatggtctgagcagagtgactacctggagctgcgggtgaaaggtgaggacgtcacctgggccctgttaacctattgtggtggtgatagagacgaatccgactactacatggacgtctggggcaaagggaccacggtcaccgtctcctca

SEQ ID NO: 263 - DNA, 333 bp, Artificial Sequence
  MGC28 light chain nuc
cagactgtggtgactcaggagccctcactgactgtgtccccaggagggacagtcactctcacctgtgcttccagtactggatcagtcaccagtggttcctttccaaactggttccagcagacacctggacaagcacccaggtcactgatttatagtacaaccaaaaaacactcttggacccctgcccggttctcaggctctctccttgggggcaaagctgccctgacagtgtcagatacacagccggaggacgaggctgagtattactgcctactctactgtggtggtggtcaaccttgggtgttcggcggagggaccaagctgaccgtccta

SEQ ID NO: 264 - protein, 8 aa, Artificial Sequence
  MGC29 CDRH1 aa
GFNSRSYW

SEQ ID NO: 265 - protein, 8 aa, Artificial Sequence
  MGC29 CDRH2 aa
INQDGTEK

SEQ ID NO: 266 - protein, 136 aa, Artificial Sequence; identical to SEQ ID NO: 176
  MGC29 CDRH3 aa

SEQ ID NO: 267 - protein, 9 aa, Artificial Sequence
  MGC29 CDRL1 aa
TGPVTSAYY

SEQ ID NO: 268 - skipped

SEQ ID NO: 269 - protein, 9 aa, Artificial Sequence
  MGC29 CDRL2 long aa
LIYNINKKH

SEQ ID NO: 270 - protein, 11 aa, Artificial Sequence
  MGC29 CDRL3 aa
LLSCGGAQPWV

SEQ ID NO: 271 - DNA, 24 bp, Artificial Sequence
  MGC29 CDRH1 nuc
ggattcaactctcgtagttattgg

SEQ ID NO: 272 - DNA, 24 bp, Artificial Sequence
  MGC29 CDRH2 nuc
ataaatcaagatggaactgagaaa

SEQ ID NO: 273 - DNA, 408 bp, Artificial Sequence; identical to SEQ ID NO: 183
  MGC29 CDRH3 nuc

SEQ ID NO: 274 - DNA, 27 bp, Artificial Sequence
  MGC29 CDRL1 nuc
actggacctgtcaccagtgcttactat

SEQ ID NO: 275 - skipped

SEQ ID NO: 276 - DNA, 27 bp, Artificial Sequence
  MGC29 CDRL2 long nuc
cttatttataatataaacaaaaaacac

SEQ ID NO: 277 - DNA, 33 bp, Artificial Sequence; identical to SEQ ID NO: 169
  MGC29 CDRL3 nuc

SEQ ID NO: 278 - protein, 243 aa, Artificial Sequence; identical to SEQ ID NO: 188
  MGC29 heavy chain aa

SEQ ID NO: 279 - protein, 111 aa, Artificial Sequence; identical to SEQ ID NO: 189
  MGC29 light chain aa

SEQ ID NO: 280 - DNA, 729 bp, Artificial Sequence
  MGC29 heavy chain nuc
gaggtgcagctggtggagtctgggggaggcttggtccagcctggggggtccctgagactctcctgtgaagcctctggattcaactctcgtagttattggatgacctgggtccgccaggctccagggaaggggctggagtgggtggccactataaatcaagatggaactgagaaaaattatgtggactctgtgaggggccggttcaccatctccagagacaccgccaagaactcactgtttctgcaaatgaacagcctgagagccgaggacacggctgtatattactgcgcgagagacagattctgtggtggtgagagtcacttgcacggagaagaagatctgcccagaccctccatctcggctgagccaggcaccgtgatccccctggggagccatgtgactttcgtgtgccggggccccgttggggttcacacattccgcctggagagggggtggagatacaacgacactgaagatgtgtctcaagctggtccatctcagtcagaggccagattccgcattgactcggtaagggaaggaaatgccgggctttatcgatgcctctattacataccccctaaatggtctgagcagagtgactacctggaactgcgggtgaaaggtggggacgtcacctgggccctgttaacgtactgtggtggtgatggagaggaatccgactaccccatggacgtctggggcaaagggaccacggtcaccgtctcctca

SEQ ID NO: 281 - DNA, 333 bp, Artificial Sequence; identical to SEQ ID NO: 191
  MGC29 light chain nuc

SEQ ID NO: 282 - protein, 8 aa, Artificial Sequence
  MGC32 CDRH1 aa
GFNFRKSW

SEQ ID NO: 283 - protein, 8 aa, Artificial Sequence
  MGC32 CDRH2 aa
IREDGSES

SEQ ID NO: 284 - protein, 139 aa, Artificial Sequence; identical to SEQ ID NO: 122
  MGC32 CDRH3 aa

SEQ ID NO: 285 - protein, 12 aa, Artificial Sequence
  MGC32 CDRL1 aa
QSVLYRSKNKNY

SEQ ID NO: 286 - skipped

SEQ ID NO: 287 - protein, 9 aa, Artificial Sequence
  MGC32 CDRL2 long aa
LIYSTSTRA

SEQ ID NO: 288 - protein, 9 aa, Artificial Sequence
  MGC32 CDRL3 aa
LQYYITPYT

SEQ ID NO: 289 - DNA, 24 bp, Artificial Sequence
  MGC32 CDRH1 nuc
gggttcaactttagaaagtcttgg

SEQ ID NO: 290 - DNA, 24 bp, Artificial Sequence
  MGC32 CDRH2 nuc
ataagagaagatggaagtgagagt

SEQ ID NO: 291 - DNA, 417 bp, Artificial Sequence; identical to SEQ ID NO: 129
  MGC32 CDRH3 nuc

SEQ ID NO: 292 - DNA, 36 bp, Artificial Sequence; identical to SEQ ID NO: 130
  MGC32 CDRL1 nuc

SEQ ID NO: 293 - skipped

SEQ ID NO: 294 - DNA, 27 bp, Artificial Sequence
  MGC32 CDRL2 long nuc
ctcatttactcgacatctactcgggcg

SEQ ID NO: 295 - DNA, 27 bp, Artificial Sequence
  MGC32 CDRL3 nuc
ctgcaatattatattactccctacact

SEQ ID NO: 296 - protein, 246 aa, Artificial Sequence; identical to SEQ ID NO: 134
  MGC32 heavy chain aa

SEQ ID NO: 297 - protein, 113 aa, Artificial Sequence
  MGC32 light chain aa
DILMTQSPDSLAVSLGERATINCKSSQSVLYRSKNKNYLAWFQQKPGQPPKVLIYSTSTRASGVPDRFTGSGSGTDFTLTISSLQAEDVAVYYCLQYYITPYTFGQGTKLEIK

SEQ ID NO: 298 - DNA, 738 bp, Artificial Sequence; identical to SEQ ID NO: 136
  MGC32 heavy chain nuc

SEQ ID NO: 299 - DNA, 339 bp, Artificial Sequence
  MGC32 light chain nuc
gacatcctcatgacccagtctccagactccctggctgtgtctctgggcgagagggccaccatcaactgcaagtccagtcagagtgttttatacaggtccaagaataagaactacttagcttggttccagcagaaaccaggacagcctcctaaggtgctcatttactcgacatctactcgggcgtccggggtccctgaccgattcactggcagcgggtctgggacagatttcactctcaccatcagcagcctgcaggctgaagatgtggcagtttattactgtctgcaatattatattactccctacacttttggccaggggaccaagttggagatcaaa

SEQ ID NO: 300 - protein, 8 aa, Artificial Sequence
  MGC33 CDRH1 aa
GFTFSSYW

SEQ ID NO: 301 - protein, 8 aa, Artificial Sequence
  MGC33 CDRH2 aa
INQDGSER

SEQ ID NO: 302 - protein, 137 aa, Artificial Sequence; identical to SEQ ID NO: 248
  MGC33 CDRH3 aa

SEQ ID NO: 303 - protein, 9 aa, Artificial Sequence
  MGC33 CDRL1 aa
TGSVTSGSF

SEQ ID NO: 304 - skipped

SEQ ID NO: 305 - protein, 9 aa, Artificial Sequence
  MGC33 CDRL2 long aa
LIYSTTKKH

SEQ ID NO: 306 - protein, 11 aa, Artificial Sequence
  MGC33 CDRL3 aa
LLYCGGGQPWV

SEQ ID NO: 307 - DNA, 24 bp, Artificial Sequence
  MGC33 CDRH1 nuc
ggattcacgtttagttcttattgg

SEQ ID NO: 308 - DNA, 24 bp, Artificial Sequence
  MGC33 CDRH2 nuc
ataaaccaagatggaagtgagaga

SEQ ID NO: 309 - DNA, 411 bp, Artificial Sequence; identical to SEQ ID NO: 255
  MGC33 CDRH3 nuc

SEQ ID NO: 310 - DNA, 27 bp, Artificial Sequence
  MGC33 CDRL1 nuc
actggatcagtcaccagtggttccttt

SEQ ID NO: 311 - skipped

SEQ ID NO: 312 - DNA, 27 bp, Artificial Sequence
  MGC33 CDRL2 long nuc
ctgatttatagtacaaccaaaaaacac

SEQ ID NO: 313 - DNA, 33 bp, Artificial Sequence; identical to SEQ ID NO: 259
  MGC33 CDRL3 nuc

SEQ ID NO: 314 - protein, 244 aa, Artificial Sequence
  MGC33 heavy chain aa
EVHLVESGGGLVQPGGSLRLSCEASGFTFSSYWMTWVRQAPGKGLEWVANINQDGSERYYVDSVKGRFTISRDTVKNSLYLQMNNLRAEDTAVYYCARQRFCSDGSLFHGEDLPRPTISAETGTVISLGSHVTFVCRGPLGVQTFRLERESRSRYSETEDVSQVGPSESEARFRIDSVSEGNAGLYRCIYYKPPKWSEQSDYLELRVKGEDVTWALLTYCGGDRDESDYYMDVWGKGTTVTVSS

SEQ ID NO: 315 - protein, 111 aa, Artificial Sequence
  MGC33 light chain aa
QTVVTQEPSLTVSPGGTVTLTCASSTGSVTSGSFPNWFQQTPGQAPRSLIYSTTKKHSWTPARFSGSLLGGKAALTVSDTQPEDEAEYYCLLYCGGGQPWVFGGGTKLTVQ

SEQ ID NO: 316 - DNA, 732 bp, Artificial Sequence
  MGC33 heavy chain nuc
gaggtgcacctggtggagtctgggggaggcttggtccagccgggggggtccctgagactctcctgtgaagcctctggattcacgtttagttcttattggatgacctgggtccgccaggctccagggaaggggctggagtgggtggccaatataaaccaagatggaagtgagagatattatgtggactctgtgaagggccggttcaccatctccagagacaccgtcaagaactcactgtatttgcaaatgaacaacctgagagccgaggacacggctgtatattactgcgcgagacaaagattctgcagtgatgggagtctctttcacggagaagatctgcccagacccaccatctcggctgagacaggcaccgtgatctccctggggagccatgtgactttcgtgtgccggggcccacttggggtgcaaacattccgcctggagagggagagtaggtccagatacagtgaaactgaagatgtgtctcaagttggtccatctgagtcagaggccagattccgcattgactcagtgagtgaaggaaatgccgggctttatcgatgcatctattacaaaccccctaaatggtctgagcagagtgactacctggagctgcgggtgaaaggtgaggacgtcacctgggccctgttaacctattgtggtggtgatagagacgaatccgactactacatggacgtctggggcaaagggaccacggtcaccgtctcctca

SEQ ID NO: 317 - DNA, 333 bp, Artificial Sequence
  MGC33 light chain nuc
cagactgtggtgactcaggagccctcactgactgtgtccccaggagggacagtcactctcacctgtgcttccagtactggatcagtcaccagtggttcctttccaaactggttccagcagacacctggacaagcacccaggtcactgatttatagtacaaccaaaaaacactcttggacccctgcccggttctcaggctctctccttgggggcaaagctgccctgacagtgtcagatacacagccggaggacgaggctgagtattactgcctactctactgtggtggtggtcaaccttgggtgttcggcggagggaccaagctgaccgtccaa

SEQ ID NO: 318 - protein, 8 aa, Artificial Sequence
  MGC34 CDRH1 aa
GFTFSSYW

SEQ ID NO: 319 - protein, 8 aa, Artificial Sequence
  MGC34 CDRH2 aa
INQDGSQK

SEQ ID NO: 320 - protein, 138 aa, Artificial Sequence
  MGC34 CDRH3 aa
ARERLCTDDSHMHGEEDLPRPSISAEPGTVIPLGSHVTFVCRGPVGIHTFRLERESRSLYTETEDVTQVSPSESEARFRIESVTEGNAGLYRCVYYKPPKWSEQSDYLELLVKGEDVTRALFTHCGGDGKESDYHMDV

SEQ ID NO: 321 - protein, 9 aa, Artificial Sequence
  MGC34 CDRL1 aa
TGAVTSGYY

SEQ ID NO: 322 - skipped

SEQ ID NO: 323 - protein, 9 aa, Artificial Sequence
  MGC34 CDRL2 long aa
LIYSTSKTH

SEQ ID NO: 324 - protein, 11 aa, Artificial Sequence
  MGC34 CDRL3 aa
LLYYGGPQPWV

SEQ ID NO: 325 - DNA, 24 bp, Artificial Sequence
  MGC34 CDRH1 nuc
ggattcacctttagtagttattgg

SEQ ID NO: 326 - DNA, 24 bp, Artificial Sequence
  MGC34 CDRH2 nuc
ataaaccaagatggaagtcagaaa

SEQ ID NO: 327 - DNA, 414 bp, Artificial Sequence
  MGC34 CDRH3 nuc
gcgagagaaagattgtgcactgatgatagtcacatgcacggagaagaagatctgcccagaccctccatctcggctgagccaggcaccgtgatccccctggggagtcatgtgaccttcgtgtgccggggcccggttgggattcacacattccgcctggagagggagagtagatccctatacactgaaactgaagatgtgactcaagtaagtccttctgagtcagaggccagattccgcattgagtcagtaactgaaggaaatgccgggctttatcgctgcgtctattataagccccctaaatggtctgagcagagtgactacctggagctgctggtgaaaggtgaggacgtcacccgggccctgttcacccactgtggtggtgatggaaaggagtccgactaccacatggacgtc

SEQ ID NO: 328 - DNA, 27 bp, Artificial Sequence
  MGC34 CDRL1 nuc
actggagcagtcaccagtggttactat

SEQ ID NO: 329 - skipped

SEQ ID NO: 330 - DNA, 27 bp, Artificial Sequence
  MGC34 CDRL2 long nuc
ctgatttatagtacaagcaaaacacac

SEQ ID NO: 331 - DNA, 33 bp, Artificial Sequence
  MGC34 CDRL3 nuc
ctgctctattatggtggtcctcagccttgggtg

SEQ ID NO: 332 - protein, 245 aa, Artificial Sequence
  MGC34 heavy chain aa
EVQLVESGGGLVQPGGSLRLSCEASGFTFSSYWMSWVRQAPGKGLEWVANINQDGSQKDYVDSVKGRFTISRDTAKNSLYLQMNSLRAEDTAVYYCARERLCTDDSHMHGEEDLPRPSISAEPGTVIPLGSHVTFVCRGPVGIHTFRLERESRSLYTETEDVTQVSPSESEARFRIESVTEGNAGLYRCVYYKPPKWSEQSDYLELLVKGEDVTRALFTHCGGDGKESDYHMDVWGKGTTVTVSS

SEQ ID NO: 333 - protein, 111 aa, Artificial Sequence
  MGC34 light chain aa
QTVVTQEPSLTVSPGGTVTLTCASSTGAVTSGYYPSWFHQKPGQPVRALIYSTSKTHSWTPARFSGSLLGGKAALTLSNVQPEDEADYYCLLYYGGPQPWVFGGGTKLTVQ

SEQ ID NO: 334 - DNA, 735 bp, Artificial Sequence
  MGC34 heavy chain nuc
gaggtgcagctggtggagtctgggggaggcttggtccagcctggggggtcactgagactctcctgtgaagcctccggattcacctttagtagttattggatgagctgggtccgccaggctccagggaaggggctggagtgggtggccaatataaaccaagatggaagtcagaaagattatgtggattctgtgaagggccgattcaccatctccagagacaccgccaagaattcattatatctccaaatgaacagcctgagagccgaggacacggctgtttactactgtgcgagagaaagattgtgcactgatgatagtcacatgcacggagaagaagatctgcccagaccctccatctcggctgagccaggcaccgtgatccccctggggagtcatgtgaccttcgtgtgccggggcccggttgggattcacacattccgcctggagagggagagtagatccctatacactgaaactgaagatgtgactcaagtaagtccttctgagtcagaggccagattccgcattgagtcagtaactgaaggaaatgccgggctttatcgctgcgtctattataagccccctaaatggtctgagcagagtgactacctggagctgctggtgaaaggtgaggacgtcacccgggccctgttcacccactgtggtggtgatggaaaggagtccgactaccacatggacgtctggggcaaagggaccacggtcaccgtctcctca

SEQ ID NO: 335 - DNA, 333 bp, Artificial Sequence
  MGC34 light chain nuc
cagactgtggtgactcaggagccctcactgactgtgtccccaggagggacagtcactctcacctgtgcttctagcactggagcagtcaccagtggttactatccaagctggttccaccagaaacctggacaaccagtcagggcactgatttatagtacaagcaaaacacactcctggacccctgcccgcttctcaggctccctccttgggggcaaagctgccctgacactgtcaaatgtccagcctgaggacgaggctgactattactgcctgctctattatggtggtcctcagccttgggtgttcggcggagggaccaagctgaccgtccaa

SEQ ID NO: 336 - protein, 8 aa, Artificial Sequence
  MGC35 CDRH1 aa
GFNSRSYW

SEQ ID NO: 337 - protein, 8 aa, Artificial Sequence
  MGC35 CDRH2 aa
INQDATEK

SEQ ID NO: 338 - protein, 136 aa, Artificial Sequence
  MGC35 CDRH3 aa
ARDRFCGGESHLHGQEDLPRPSISAEPGSVIPLGSLVTFVCRGPVGVHTFRLERGWTYNDTEDVSQAGPSESEARFRMDSVREGNAGLYRCIYYKPPKWSEQSAYLELRVKGGDVTWALLTYCGGDGEESDYPMDV

SEQ ID NO: 339 - protein, 9 aa, Artificial Sequence
  MGC35 CDRL1 aa
TGPVTSAYY

SEQ ID NO: 340 - skipped

SEQ ID NO: 341 - protein, 9 aa, Artificial Sequence
  MGC35 CDRL2 long aa
LIYNINKKH

SEQ ID NO: 342 - protein, 11 aa, Artificial Sequence
  MGC35 CDRL3 aa
LLSCGGAQPWV

SEQ ID NO: 343 - DNA, 24 bp, Artificial Sequence
  MGC35 CDRH1 nuc
ggattcaactctcgtagttattgg

SEQ ID NO: 344 - DNA, 24 bp, Artificial Sequence
  MGC35 CDRH2 nuc
ataaatcaagatgcaactgagaaa

SEQ ID NO: 345 - DNA, 408 bp, Artificial Sequence
  MGC35 CDRH3 nuc
gcgagagacagattctgtggtggtgagagtcacttgcacggacaagaagatctgcccagaccctccatctcggctgagccaggctccgtgatccccctggggagccttgtgactttcgtgtgccggggcccggttggggttcacacattccgcctcgagagggggtggacatacaacgacactgaagatgtgtctcaagctggtccatctgagtcagaggccagattccgcatggactcggtaagggaaggaaatgccgggctttatcgatgcatctattacaaaccccctaaatggtctgagcagagtgcctacctggaactgcgggtgaaaggtggggacgtcacctgggccctgttaacgtactgtggtggtgatggagaggaatccgactaccccatggacgtc

SEQ ID NO: 346 - DNA, 27 bp, Artificial Sequence
  MGC35 CDRL1 nuc
actggacctgtcaccagtgcttactat

SEQ ID NO: 347 - skipped

SEQ ID NO: 348 - DNA, 27 bp, Artificial Sequence
  MGC35 CDRL2 long nuc
cttatttataatataaacaaaaaacac

SEQ ID NO: 349 - DNA, 33 bp, Artificial Sequence; identical to SEQ ID NO: 169
  MGC35 CDRL3 nuc

SEQ ID NO: 350 - protein, 243 aa, Artificial Sequence
  MGC35 heavy chain aa
EVQLVESGGGLVQPGGSLRLSCEASGFNSRSYWMTWVRQAPGKGLEWVASINQDATEKNYVDSVKGRFTISRDTAKNSLYLQMNSLRAEDTAVYYCARDRFCGGESHLHGQEDLPRPSISAEPGSVIPLGSLVTFVCRGPVGVHTFRLERGWTYNDTEDVSQAGPSESEARFRMDSVREGNAGLYRCIYYKPPKWSEQSAYLELRVKGGDVTWALLTYCGGDGEESDYPMDVWGKGTTVTVSS

SEQ ID NO: 351 - protein, 111 aa, Artificial Sequence
  MGC35 light chain aa
QTVVTQEPSLTVSPGGTVTLTCASSTGPVTSAYYPNWFQQKPGQAPRSLIYNINKKHSWTPDRFSGSLLGGKAALTLSGVQPEDEADYYCLLSCGGAQPWVFGGGTKLTVQ

SEQ ID NO: 352 - DNA, 729 bp, Artificial Sequence
  MGC35 heavy chain nuc
gaggtgcaactggtggagtctgggggaggcttggtccagcctggggggtccctgagactctcctgtgaagcctctggattcaactctcgtagttattggatgacctgggtccgccaggctccagggaaggggctggagtgggtggccagtataaatcaagatgcaactgagaaaaattatgtggactctgtgaagggccggttcaccatctccagagacaccgccaagaactcactgtatctgcaaatgaacagcctgagagccgaggacacggctgtatattactgcgcgagagacagattctgtggtggtgagagtcacttgcacggacaagaagatctgcccagaccctccatctcggctgagccaggctccgtgatccccctggggagccttgtgactttcgtgtgccggggcccggttggggttcacacattccgcctcgagagggggtggacatacaacgacactgaagatgtgtctcaagctggtccatctgagtcagaggccagattccgcatggactcggtaagggaaggaaatgccgggctttatcgatgcatctattacaaaccccctaaatggtctgagcagagtgcctacctggaactgcgggtgaaaggtggggacgtcacctgggccctgttaacgtactgtggtggtgatggagaggaatccgactaccccatggacgtctggggcaaagggaccacggtcaccgtctcctca

SEQ ID NO: 353 - DNA, 333 bp, Artificial Sequence
  MGC35 light chain nuc
cagactgtggtgactcaggagccctcactgactgtgtccccaggagggacagtcactctcacctgtgcttccagcactggacctgtcaccagtgcttactatccaaactggttccagcagaagcctggacaagcacccaggtctcttatttataatataaacaaaaaacactcctggacccctgaccggttctcaggctccctccttgggggcaaagctgccctgacactgtcaggtgtacagcctgaggacgaggctgactattactgcctgctctcctgtggtggtgctcagccttgggtgttcggcggagggaccaagctgaccgtccaa

SEQ ID NO: 354 - protein, 8 aa, Artificial Sequence
  MGC36 CDRH1 aa
GFNSRSYW

SEQ ID NO: 355 - protein, 8 aa, Artificial Sequence
  MGC36 CDRH2 aa
INQDGTEK

SEQ ID NO: 356 - protein, 136 aa, Artificial Sequence; identical to SEQ ID NO: 158
  MGC36 CDRH3 aa

SEQ ID NO: 357 - protein, 9 aa, Artificial Sequence
  MGC36 CDRL1 aa
TGPVTSAYY

SEQ ID NO: 358 - skipped

SEQ ID NO: 359 - protein, 9 aa, Artificial Sequence
  MGC36 CDRL2 long aa
LIYSINKKH

SEQ ID NO: 360 - protein, 11 aa, Artificial Sequence
  MGC36 CDRL3 aa
LLSCGGAQPWV

SEQ ID NO: 361 - DNA, 24 bp, Artificial Sequence
  MGC36 CDRH1 nuc
ggattcaactctcgtagttattgg

SEQ ID NO: 362 - DNA, 24 bp, Artificial Sequence
  MGC36 CDRH2 nuc
ataaatcaagatgggactgagaaa

SEQ ID NO: 363 - DNA, 408 bp, Artificial Sequence; identical to SEQ ID NO: 165
  MGC36 CDRH3 nuc

SEQ ID NO: 364 - DNA, 27 bp, Artificial Sequence
  MGC36 CDRL1 nuc
actggacctgtcaccagtgcttactat

SEQ ID NO: 365 - skipped

SEQ ID NO: 366 - DNA, 27 bp, Artificial Sequence
  MGC36 CDRL2 long nuc
cttatttatagtataaacaaaaaacac

SEQ ID NO: 367 - DNA, 33 bp, Artificial Sequence; identical to SEQ ID NO: 169
  MGC36 CDRL3 nuc

SEQ ID NO: 368 - protein, 243 aa, Artificial Sequence
  MGC36 heavy chain aa
EVVLVESGGGLVQPGGSLRLSCEASGFNSRSYWMTWVRQAPGKGLEWVASINQDGTEKNYVDSVKGRFTISRDSAKNSLYLQMSSLRADDTAVYYCARDRFCGGESHLHGEEDLPRPSISAEPDTVIPLGSHVTFVCRGPVGVHTFRLERGWRYNDTEDVSQAGPSESEARFRIDSVREGNAGLYRCIYYIAPKWSEQSDYLELRVKGGDVTWALLTYCGGDGEESDYPMDVWGKGTTVTVSS

SEQ ID NO: 369 - protein, 111 aa, Artificial Sequence; identical to SEQ ID NO: 171
  MGC36 light chain aa

SEQ ID NO: 370 - DNA, 729 bp, Artificial Sequence
  MGC36 heavy chain nuc
gaggtggtactggtggagtctgggggaggcttggtccagcctggggggtccctgagactctcctgtgaagcctctggattcaactctcgtagttattggatgacctgggtccgccaggctccagggaaggggctggagtgggtggccagtataaatcaagatgggactgagaaaaattatgtggactctgtgaagggccggttcaccatctccagagactccgccaagaactcactgtatctgcaaatgagcagcctgagagccgacgacacggctgtatattactgtgcgagagacagattctgtggtggtgagagtcacttgcacggagaagaagatctgcccagaccctccatctcggctgagccagacaccgtaatccccctggggagccatgtgactttcgtgtgccggggcccggttggggttcacacattccgcctggagagggggtggaggtacaacgacactgaagatgtgtctcaagctggtccatctgagtcagaggccagattccgcattgactcggtaagggaaggaaatgccgggctttatcgatgcatctattacatagcccctaaatggtctgagcagagtgactacctggagctgcgggtgaaaggtggggacgtcacctgggccctgttaacgtactgtggcggtgatggagaggaatccgactaccccatggacgtctggggcaaagggaccacggtcaccgtctcctca

SEQ ID NO: 371 - DNA, 333 bp, Artificial Sequence
  MGC36 light chain nuc
cagactgtggttactcaggagccctcactgactgtgtccccaggagggacagtcactctcacctgtgcttccagcactggacctgtcaccagtgcttactatccaaactggttccagcagaagcctggacaagcacccaggtctcttatttatagtataaacaaaaaacactcctggacccctgcccggttctcaggctccctccttgggggcaaagctgccctgacactgtcaggtgtacagcctgaggacgaggctgactattactgcctgctctcctgtggtggtgctcagccttgggtgttcggcggagggaccaagctgaccgtccaa

SEQ ID NO: 372 - protein, 8 aa, Artificial Sequence
  MGC37 CDRH1 aa
GFTFRNYW

SEQ ID NO: 373 - protein, 8 aa, Artificial Sequence
  MGC37 CDRH2 aa
IRQDGSEK

SEQ ID NO: 374 - protein, 138 aa, Artificial Sequence; identical to SEQ ID NO: 194
  MGC37 CDRH3 aa

SEQ ID NO: 375 - protein, 6 aa, Artificial Sequence
  MGC37 CDRL1 aa
QRLSRS

SEQ ID NO: 376 - skipped

SEQ ID NO: 377 - protein, 9 aa, Artificial Sequence
  MGC37 CDRL2 long aa
LIYKASPLE

SEQ ID NO: 378 - protein, 9 aa, Artificial Sequence
  MGC37 CDRL3 aa
QQYSNYSYS

SEQ ID NO: 379 - DNA, 24 bp, Artificial Sequence
  MGC37 CDRH1 nuc
ggattcaccttcagaaattattgg

SEQ ID NO: 380 - DNA, 24 bp, Artificial Sequence
  MGC37 CDRH2 nuc
ataaggcaagatggaagtgagaag

SEQ ID NO: 381 - DNA, 414 bp, Artificial Sequence; identical to SEQ ID NO: 201
  MGC37 CDRH3 nuc

SEQ ID NO: 382 - DNA, 18 bp, Artificial Sequence
  MGC37 CDRL1 nuc
cagcgtcttagtcgctcg

SEQ ID NO: 383 - skipped

SEQ ID NO: 384 - DNA, 27 bp, Artificial Sequence
  MGC37 CDRL2 long nuc
ctgatctataaggcgtctcctttagaa

SEQ ID NO: 385 - DNA, 27 bp, Artificial Sequence
  MGC37 CDRL3 nuc
caacaatacagtaattattcatatagt

SEQ ID NO: 386 - protein, 245 aa, Artificial Sequence; identical to SEQ ID NO: 206
  MGC37 heavy chain aa

SEQ ID NO: 387 - protein, 107 aa, Artificial Sequence; identical to SEQ ID NO: 243
  MGC37 light chain aa

SEQ ID NO: 388 - DNA, 735 bp, Artificial Sequence; identical to SEQ ID NO: 208
  MGC37 heavy chain nuc

SEQ ID NO: 389 - DNA, 321 bp, Artificial Sequence; identical to SEQ ID NO: 245
  MGC37 light chain nuc

SEQ ID NO: 390 - protein, 9 aa, Artificial Sequence
  MGD21 CDRH1 aa
GDYVNTNRR

SEQ ID NO: 391 - protein, 7 aa, Artificial Sequence
  MGD21 CDRH2 aa
VHQSGRT

SEQ ID NO: 392 - protein, 143 aa, Artificial Sequence
  MGD21 CDRH3 aa
ARASPLKSQRDTDLPRPSISAEPGTVIPLGSHVTFVCRGPVGVQTFRLERERNYLYSDTEDVSQTSPSESEARFRIDSVNAGNAGLFRCIYYKSRKWSEQSDYLELVVKGEDVTWALSQSQDDPRACPQGELPISTDIYYVDV

SEQ ID NO: 393 - protein, 6 aa, Artificial Sequence
  MGD21 CDRL1 aa
QHINDS

SEQ ID NO: 394 - skipped

SEQ ID NO: 395 - protein, 9 aa, Artificial Sequence
  MGD21 CDRL2 long aa
LIYGASNLH

SEQ ID NO: 396 - protein, 9 aa, Artificial Sequence
  MGD21 CDRL3 aa
QQCNCFPPD

SEQ ID NO: 397 - DNA, 27 bp, Artificial Sequence
  MGD21 CDRH1 nuc
ggtgactacgtcaatactaataggagg

SEQ ID NO: 398 - DNA, 21 bp, Artificial Sequence
  MGD21 CDRH2 nuc
gttcatcaaagtgggagaacc

SEQ ID NO: 399 - DNA, 429 bp, Artificial Sequence
  MGD21 CDRH3 nuc
gcgagagcgtctccactcaaatctcagagggacaccgatctgcccagaccctccatctcggctgagccgggcaccgtgatccccctggggagccatgtgactttcgtgtgccggggcccggttggggttcaaacattccgcctggagagggagaggaattatttatacagtgatactgaagatgtgtctcaaactagtccatctgagtcggaggccagattccgcattgactcagtaaatgcaggcaatgccgggctttttcgctgcatctattacaagtcccgtaaatggtctgagcagagtgactacctggagctggtggtgaaaggtgaggacgtcacctgggccctgtcccagtctcaagacgaccctcgagcttgtccccagggggagctccccataagtaccgatatttactacgtggacgtc

SEQ ID NO: 400 - DNA, 18 bp, Artificial Sequence
  MGD21 CDRL1 nuc
caacatattaatgattct

SEQ ID NO: 401 - skipped

SEQ ID NO: 402 - DNA, 27 bp, Artificial Sequence
  MGD21 CDRL2 long nuc
ctgatatatggtgcatccaatttgcac

SEQ ID NO: 403 - DNA, 27 bp, Artificial Sequence
  MGD21 CDRL3 nuc
caacagtgtaattgtttccctccggac

SEQ ID NO: 404 - protein, 250 aa, Artificial Sequence
  MGD21 heavy chain aa
EVQLVETGPGLMKTSGTLSLTCAVSGDYVNTNRRWSWVRQAPGKGLEWIGEVHQSGRTNYNPSLKSRVTISVDKSKNQFSLKVDSVTAADTAVYYCARASPLKSQRDTDLPRPSISAEPGTVIPLGSHVTFVCRGPVGVQTFRLERERNYLYSDTEDVSQTSPSESEARFRIDSVNAGNAGLFRCIYYKSRKWSEQSDYLELVVKGEDVTWALSQSQDDPRACPQGELPISTDIYYVDVWGNGTTVTVSS

SEQ ID NO: 405 - protein, 107 aa, Artificial Sequence
  MGD21 light chain aa
AIRMTQSPSSLSASPGDKVSITCRASQHINDSLAWFQQRPGKAPKLLIYGASNLHSGVPSRFSGTGSGTDFTLTITGLQSEDFATYFCQQCNCFPPDFGQGTRLEIK

SEQ ID NO: 406 - DNA, 750 bp, Artificial Sequence
  MGD21 heavy chain nuc
gaggtgcagctggtggagacgggcccaggactgatgaagacttcggggaccctgtccctcacgtgcgctgtgtctggtgactacgtcaatactaataggaggtggagttgggtccgccaggccccagggaagggcctggagtggattggagaggttcatcaaagtgggagaaccaattacaacccgtccctcaagagccgagtcaccatatcagtagacaagtctaagaatcagttctctctgaaggtggactctgtgaccgccgcggacacggccgtgtattactgtgcgagagcgtctccactcaaatctcagagggacaccgatctgcccagaccctccatctcggctgagccgggcaccgtgatccccctggggagccatgtgactttcgtgtgccggggcccggttggggttcaaacattccgcctggagagggagaggaattatttatacagtgatactgaagatgtgtctcaaactagtccatctgagtcggaggccagattccgcattgactcagtaaatgcaggcaatgccgggctttttcgctgcatctattacaagtcccgtaaatggtctgagcagagtgactacctggagctggtggtgaaaggtgaggacgtcacctgggccctgtcccagtctcaagacgaccctcgagcttgtccccagggggagctccccataagtaccgatatttactacgtggacgtctggggcaacgggaccacggtcaccgtctcctca

SEQ ID NO: 407 - DNA, 321 bp, Artificial Sequence
  MGD21 light chain nuc
gccatccggatgacccagtctccatcctcactctctgcatcaccaggggacaaagtcagcatcacttgtcgggcgagtcaacatattaatgattctttggcctggtttcaacaaaggccagggaaagccccaaaactcctgatatatggtgcatccaatttgcacagtggggtcccatcgaggttcagcggcactgggtcagggacagatttcactctcactatcaccggcctgcagtctgaagattttgcaacttatttctgtcaacagtgtaattgtttccctccggacttcggccaagggacacgactggagattaaa

SEQ ID NO: 408 - protein, 9 aa, Artificial Sequence
  MGD23 CDRH1 aa
GGSISSNKW

SEQ ID NO: 409 - protein, 7 aa, Artificial Sequence
  MGD23 CDRH2 aa
VYQTGIT

SEQ ID NO: 410 - protein, 144 aa, Artificial Sequence
  MGD23 CDRH3 aa
ATISQLRPQGDTEDLPRPSLSAEPGTVIPLGSHVTFVCRGPAGVETFRLERESRFTYNDTEDVSQASPSESEARFRIDSVSEGNAGPYRCLYYKARKWSDQSDYLELLVKGADVTWALPQSQLAPRACPQGELRISTDVFSMNV

SEQ ID NO: 411 - protein, 6 aa, Artificial Sequence
  MGD23 CDRL1 aa
QYVGNY

SEQ ID NO: 412 - skipped

SEQ ID NO: 413 - protein, 9 aa, Artificial Sequence
  MGD23 CDRL2 long aa
LIHGVSTLQ

SEQ ID NO: 414 - protein, 9 aa, Artificial Sequence
  MGD23 CDRL3 aa
QQYYTSPPD

SEQ ID NO: 415 - DNA, 27 bp, Artificial Sequence
  MGD23 CDRH1 nuc
ggtggctccattagtagtaataagtgg

SEQ ID NO: 416 - DNA, 21 bp, Artificial Sequence
  MGD23 CDRH2 nuc
gtgtatcagactggtattacc

SEQ ID NO: 417 - DNA, 432 bp, Artificial Sequence
  MGD23 CDRH3 nuc
gcgacaatttctcaactgaggccgcagggggacaccgaagatctgcccagaccctccctctcggctgaaccaggcaccgtgatccccctggggagtcacgtgactttcgtgtgccggggcccggctggggtcgaaacattccgcctggagagggagagtagattcacttacaacgatactgaagatgtgtctcaagcgagtccatctgagtcagaggccagattccgcattgactcagtaagtgaaggaaatgccgggccttatcgctgcctctattataaggcccgtaaatggtctgaccagagtgactacttggaattgctggtgaagggtgcggacgtcacctgggccctgccccagtctcagctcgcccctcgagcttgtccccagggagaactccgcattagtaccgatgttttctccatgaacgtc

SEQ ID NO: 418 - DNA, 18 bp, Artificial Sequence
  MGD23 CDRL1 nuc
caatatgttgggaattat

SEQ ID NO: 419 - skipped

SEQ ID NO: 420 - DNA, 27 bp, Artificial Sequence
  MGD23 CDRL2 long nuc
ctcattcacggtgtatccactttgcaa

SEQ ID NO: 421 - DNA, 27 bp, Artificial Sequence
  MGD23 CDRL3 nuc
cagcagtattatacttcccctccggac

SEQ ID NO: 422 - protein, 251 aa, Artificial Sequence
  MGD23 heavy chain aa
QVQLQESGPGLVKPSGTLSLTCSVSGGSISSNKWWSWVRQSPGKGLEWIGEVYQTGITNYNPSLKGRVTMSVDKSKNQFSLRLTSVTAADTAVYYCATISQLRPQGDTEDLPRPSLSAEPGTVIPLGSHVTFVCRGPAGVETFRLERESRFTYNDTEDVSQASPSESEARFRIDSVSEGNAGPYRCLYYKARKWSDQSDYLELLVKGADVTWALPQSQLAPRACPQGELRISTDVFSMNVWGNGTTVTVSS

SEQ ID NO: 423 - protein, 107 aa, Artificial Sequence
  MGD23 light chain aa
AVRVTQSPTSLSASTGDRVTITCRTSQYVGNYLDWYQQKPGKAPKLLIHGVSTLQNGVPSRFSGSASGTDFTLNITCLQSEDSATYYCQQYYTSPPDFGQGTRLEIK

SEQ ID NO: 424 - DNA, 753 bp, Artificial Sequence
  MGD23 heavy chain nuc
caggtgcagctgcaggagtcgggcccaggactggtgaagccttcgggaaccctgtccctcacctgcagtgtctctggtggctccattagtagtaataagtggtggagttgggtccgccagtccccagggaagggcctggagtggattggggaggtgtatcagactggtattaccaactacaacccgtccctcaagggtcgagtcaccatgtcagtggacaagtccaagaaccaattctccctgagactgacttctgtgaccgccgcggacacggccgtgtattactgtgcgacaatttctcaactgaggccgcagggggacaccgaagatctgcccagaccctccctctcggctgaaccaggcaccgtgatccccctggggagtcacgtgactttcgtgtgccggggcccggctggggtcgaaacattccgcctggagagggagagtagattcacttacaacgatactgaagatgtgtctcaagcgagtccatctgagtcagaggccagattccgcattgactcagtaagtgaaggaaatgccgggccttatcgctgcctctattataaggcccgtaaatggtctgaccagagtgactacttggaattgctggtgaagggtgcggacgtcacctgggccctgccccagtctcagctcgcccctcgagcttgtccccagggagaactccgcattagtaccgatgttttctccatgaacgtctggggcaacgggaccacggtcaccgtctcttca

SEQ ID NO: 425 - DNA, 321 bp, Artificial Sequence
  MGD23 light chain nuc
gccgtccgggtgacccagtctccaacctcactgtctgcatctacaggagacagagtcaccatcacttgtcggacgagtcaatatgttgggaattatttagattggtatcagcaaaaaccagggaaagcccctaaactcctcattcacggtgtatccactttgcaaaatggggtcccatcaaggttcagtggcagtgcctccgggacagacttcactctcaacatcacctgcctacagtctgaagattctgcaacttattactgtcagcagtattatacttcccctccggacttcggccaagggacacgcctggaaattaag

SEQ ID NO: 426 - protein, 9 aa, Artificial Sequence
  MGD30 CDRH1 aa
GGSITSSKW

SEQ ID NO: 427 - protein, 7 aa, Artificial Sequence
  MGD30 CDRH2 aa
IYHNGTT

SEQ ID NO: 428 - protein, 145 aa, Artificial Sequence
  MGD30 CDRH3 aa
ATASPFKSHHRTTEKLPRPSISAEPGTVIPLGSRVTFVCRGPVGVQTFRLERETSFTYNDTEDVSQVSPSESEARFRIDSVSEGYAGPYRCVYYKAPKWSEQSDYLDLLVKGEDVTWALTQPQLDPRACPQGDLRMSTDIYCMDV

SEQ ID NO: 429 - protein, 6 aa, Artificial Sequence
  MGD30 CDRL1 aa
QDISTF

SEQ ID NO: 430 - skipped

SEQ ID NO: 431 - protein, 9 aa, Artificial Sequence
  MGD30 CDRL2 long aa
LIFAASTLQ

SEQ ID NO: 432 - protein, 9 aa, Artificial Sequence
  MGD30 CDRL3 aa
QQYYCFPPD

SEQ ID NO: 433 - DNA, 27 bp, Artificial Sequence
  MGD30 CDRH1 nuc
ggtggctccatcaccagtagtaagtgg

SEQ ID NO: 434 - DNA, 21 bp, Artificial Sequence
  MGD30 CDRH2 nuc
atctatcataatgggaccacc

SEQ ID NO: 435 - DNA, 435 bp, Artificial Sequence
  MGD30 CDRH3 nuc
gcaacggcgtctcccttcaagtctcatcacaggaccaccgaaaaactgcccagaccctccatctcggctgagccgggcaccgtgatccccctggggagccgtgtgactttcgtgtgccggggcccggttggggttcaaacattccgcctagagagggagactagctttacatataatgatactgaagatgtgtctcaggttagtccgtctgagtcagaggccagattccgcattgactcagtgagtgagggatatgccgggccttatcgctgcgtctattataaggcccctaagtggtccgagcagagtgactacctggacctgctggtgaaaggtgaggacgtcacttgggccctgacccagcctcagctcgaccctcgagcttgtccccagggggacctccgcatgagcaccgatatttactgcatggacgtc

SEQ ID NO: 436 - DNA, 18 bp, Artificial Sequence
  MGD30 CDRL1 nuc
caggatattagcactttt

SEQ ID NO: 437 - skipped

SEQ ID NO: 438 - DNA, 27 bp, Artificial Sequence
  MGD30 CDRL2 long nuc
ctaatctttgctgcatctactttacaa

SEQ ID NO: 439 - DNA, 27 bp, Artificial Sequence
  MGD30 CDRL3 nuc
caacagtattattgtttccctccggac

SEQ ID NO: 440 - protein, 252 aa, Artificial Sequence
  MGD30 heavy chain aa
QVQLQESGPGLVKPSETLSLSCAVTGGSITSSKWWTWVRQGPDKGLEWIGKIYHNGTTNYNPSLKSRVAMSVDKSRNQFSLRLTSVTAADTALYYCATASPFKSHHRTTEKLPRPSISAEPGTVIPLGSRVTFVCRGPVGVQTFRLERETSFTYNDTEDVSQVSPSESEARFRIDSVSEGYAGPYRCVYYKAPKWSEQSDYLDLLVKGEDVTWALTQPQLDPRACPQGDLRMSTDIYCMDVWGKGTTVTVSS

SEQ ID NO: 441 - protein, 107 aa, Artificial Sequence
  MGD30 light chain aa
AIRLTQSPSSLSASIGDRVTITCRASQDISTFLAWYQQESGKAPRLLIFAASTLQTGVPSRFSGSGSGTDFTLTISGLQSEDFATYYCQQYYCFPPDFGQGTRLDIK

SEQ ID NO: 442 - DNA, 756 bp, Artificial Sequence
  MGD30 heavy chain nuc
caggtgcagctgcaggagtcgggcccaggactggtgaagccttcagaaaccctgtccctctcctgcgctgtcactggtggctccatcaccagtagtaagtggtggacttgggtccgccagggcccagataaggggctggagtggattgggaaaatctatcataatgggaccaccaactacaatccgtccctcaagagtcgagtcgccatgtcggtggacaagtccaggaaccagttctccctgagactgacctccgtgaccgccgcggacacggccttgtattactgtgcaacggcgtctcccttcaagtctcatcacaggaccaccgaaaaactgcccagaccctccatctcggctgagccgggcaccgtgatccccctggggagccgtgtgactttcgtgtgccggggcccggttggggttcaaacattccgcctagagagggagactagctttacatataatgatactgaagatgtgtctcaggttagtccgtctgagtcagaggccagattccgcattgactcagtgagtgagggatatgccgggccttatcgctgcgtctattataaggcccctaagtggtccgagcagagtgactacctggacctgctggtgaaaggtgaggacgtcacttgggccctgacccagcctcagctcgaccctcgagcttgtccccagggggacctccgcatgagcaccgatatttactgcatggacgtctggggcaaagggaccacggtcaccgtctcctca

SEQ ID NO: 443 - DNA, 321 bp, Artificial Sequence
  MGD30 light chain nuc
gccatccggttgacccaatctccatcctcactctctgcatctataggagacagagtcaccatcacttgtcgggcgagtcaggatattagcacttttttggcctggtatcaacaagagtcaggtaaagccccaaggctcctaatctttgctgcatctactttacaaactggggtcccttcaaggttcagcggcagtggatctgggacagatttcactctcaccatcagcggcctgcaatctgaagattttgcaacttattactgtcaacagtattattgtttccctccggacttcggccaagggacacgactggacattaaa

SEQ ID NO: 444 - protein, 9 aa, Artificial Sequence
  MGD33 CDRH1 aa
GGSITSSKW

SEQ ID NO: 445 - protein, 7 aa, Artificial Sequence
  MGD33 CDRH2 aa
IYHNGTT

SEQ ID NO: 446 - protein, 145 aa, Artificial Sequence
  MGD33 CDRH3 aa
ATASPFKSHHRTTEKLPRPSISAEPGTVIPLGSRVTFVCRGPVGVQTFRLERETRSTYNDTEDVSQVSPSESEARFRIDSVSEGYAGPYRCVYYKAPKWSEQSDYLDLLVKGEDVTWALTQPQLDPRACPQGDLRMSTDIYCMDV

SEQ ID NO: 447 - protein, 6 aa, Artificial Sequence
  MGD33 CDRL1 aa
QDISTY

SEQ ID NO: 448 - skipped

SEQ ID NO: 449 - protein, 9 aa, Artificial Sequence
  MGD33 CDRL2 long aa
LIFAASSLQ

SEQ ID NO: 450 - protein, 9 aa, Artificial Sequence
  MGD33 CDRL3 aa
QQYYCFPPD

SEQ ID NO: 451 - DNA, 27 bp, Artificial Sequence
  MGD33 CDRH1 nuc
ggtggctccatcaccagtagtaagtgg

SEQ ID NO: 452 - DNA, 21 bp, Artificial Sequence
  MGD33 CDRH2 nuc
atctatcataatgggaccacc

SEQ ID NO: 453 - DNA, 435 bp, Artificial Sequence
  MGD33 CDRH3 nuc
gcaacggcgtctcccttcaagtctcatcacaggaccaccgaaaaactgcccagaccctccatctcggctgagccgggcaccgtgatccccctggggagccgtgtgactttcgtgtgccggggcccggttggggttcaaacattccgcctagagagggagactagatctacatataatgatactgaagatgtgtctcaggttagtccgtctgagtcagaggccagattccgcattgactcagtgagtgagggatatgccgggccttatcgctgcgtctattataaggcccctaagtggtccgagcagagtgactacctggacctgctggtgaaaggtgaggacgtcacttgggccctgacccagcctcagctcgaccctcgagcttgtccccagggggacctccgcatgagcaccgatatttactgcatggacgtc

SEQ ID NO: 454 - DNA, 18 bp, Artificial Sequence
  MGD33 CDRL1 nuc
caggatattagcacttat

SEQ ID NO: 455 - skipped

SEQ ID NO: 456 - DNA, 27 bp, Artificial Sequence
  MGD33 CDRL2 long nuc
ctaatctttgctgcatctagtttacaa

SEQ ID NO: 457 - DNA, 27 bp, Artificial Sequence
  MGD33 CDRL3 nuc
caacaatattattgtttccctccggac

SEQ ID NO: 458 - protein, 252 aa, Artificial Sequence
  MGD33 heavy chain aa
HVQLQESGPGLVKPSETLSLSCAVTGGSITSSKWWTWVRQGPDKGLEWIGKIYHNGTTNYNPSLKSRVAMSVDKSKNQFSLRLTSVTAADTAVYYCATASPFKSHHRTTEKLPRPSISAEPGTVIPLGSRVTFVCRGPVGVQTFRLERETRSTYNDTEDVSQVSPSESEARFRIDSVSEGYAGPYRCVYYKAPKWSEQSDYLDLLVKGEDVTWALTQPQLDPRACPQGDLRMSTDIYCMDVWGKGTTVTVSS

SEQ ID NO: 459 - protein, 107 aa, Artificial Sequence
  MGD33 light chain aa
AIRLTQSPSSLSASIGDRVTITCRASQDISTYLAWYQQESGKAPRLLIFAASSLQTGVPSRFSGSGSGTDFTLTISGLQSEDFATYYCQQYYCFPPDFGQGTRLDIK

SEQ ID NO: 460 - DNA, 756 bp, Artificial Sequence
  MGD33 heavy chain nuc
cacgtgcagctgcaggagtcgggcccaggactggtgaagccttcagaaaccctgtccctctcctgcgctgtcactggtggctccatcaccagtagtaagtggtggacttgggtccgccagggcccagataaggggctggagtggattgggaaaatctatcataatgggaccaccaactacaatccgtccctcaagagtcgagtcgccatgtcggtggacaagtccaagaaccagttctccctgagactgacctccgtgaccgccgcggacacggccgtgtattactgtgcaacggcgtctcccttcaagtctcatcacaggaccaccgaaaaactgcccagaccctccatctcggctgagccgggcaccgtgatccccctggggagccgtgtgactttcgtgtgccggggcccggttggggttcaaacattccgcctagagagggagactagatctacatataatgatactgaagatgtgtctcaggttagtccgtctgagtcagaggccagattccgcattgactcagtgagtgagggatatgccgggccttatcgctgcgtctattataaggcccctaagtggtccgagcagagtgactacctggacctgctggtgaaaggtgaggacgtcacttgggccctgacccagcctcagctcgaccctcgagcttgtccccagggggacctccgcatgagcaccgatatttactgcatggacgtctggggcaaagggaccacggtcaccgtctcctca

SEQ ID NO: 461 - DNA, 321 bp, Artificial Sequence
  MGD33 light chain nuc
gccatccggttgacccaatctccatcctcactctctgcatctataggagacagagtcaccatcacttgtcgggcgagtcaggatattagcacttatttggcctggtatcaacaagagtcaggtaaagccccaaggctcctaatctttgctgcatctagtttacaaactggggtcccttcaaggttcagcggcagtggatctgggacagatttcactctcaccatcagcggcctgcagtctgaagattttgcaacttattactgtcaacaatattattgtttccctccggacttcggccaagggacacgactggacattaaa

SEQ ID NO: 462 - protein, 9 aa, Artificial Sequence
  MGD34 CDRH1 aa
GDYVNTNRR

SEQ ID NO: 463 - protein, 7 aa, Artificial Sequence
  MGD34 CDRH2 aa
VHQSGRT

SEQ ID NO: 464 - protein, 144 aa, Artificial Sequence
  MGD34 CDRH3 aa
ARASPLKSQRDTEDLPRPSISAEPGTVIPLGSHVTFVCRGPVGVQTFRLERERNYLYSDTEDVSQTSPSESEARFRIDSVNAGNAGLFRCIYYKSRKWSEQSDYLELVVKGEDVTWALSQSQVDPRACPQGELPISTDIYYVDV

SEQ ID NO: 465 - protein, 6 aa, Artificial Sequence
  MGD34 CDRL1 aa
QHINDS

SEQ ID NO: 466 - skipped

SEQ ID NO: 467 - protein, 9 aa, Artificial Sequence
  MGD34 CDRL2 long aa
LIYGASNLH

SEQ ID NO: 468 - protein, 9 aa, Artificial Sequence
  MGD34 CDRL3 aa
QQCNCFPPD

SEQ ID NO: 469 - DNA, 27 bp, Artificial Sequence
  MGD34 CDRH1 nuc
ggtgactacgtcaatactaataggagg

SEQ ID NO: 470 - DNA, 21 bp, Artificial Sequence
  MGD34 CDRH2 nuc
gttcatcaaagtgggagaacc

SEQ ID NO: 471 - DNA, 432 bp, Artificial Sequence
  MGD34 CDRH3 nuc
gcgagagcgtctccactcaaatctcagagggacaccgaagatctgcccagaccctccatctcggctgagccgggcaccgtgatccccctggggagccatgtgactttcgtgtgccggggcccggttggggttcaaacattccgcctggagagggagaggaattatttatacagtgatactgaagatgtgtctcaaactagtccatctgagtcggaggccagattccgcattgactcagtaaatgcaggcaatgccgggctttttcgctgcatctattacaagtcccgtaaatggtctgagcagagtgactacctggagctggtggtgaaaggtgaggacgtcacctgggccctgtcccagtctcaagtcgaccctcgagcttgtccccagggggagctccccataagtaccgatatttactacgtggacgtc

SEQ ID NO: 472 - DNA, 18 bp, Artificial Sequence
  MGD34 CDRL1 nuc
caacatattaatgattct

SEQ ID NO: 473 - skipped

SEQ ID NO: 474 - DNA, 27 bp, Artificial Sequence
  MGD34 CDRL2 long nuc
ctgatatatggtgcatccaatttgcac

SEQ ID NO: 475 - DNA, 27 bp, Artificial Sequence
  MGD34 CDRL3 nuc
caacagtgtaattgtttccctccggac

SEQ ID NO: 476 - protein, 251 aa, Artificial Sequence [sequence omitted]

SEQ ID NO: 477 - protein, 107 aa, Artificial Sequence; identical to SEQ ID NO: 405
  MGD34 light chain aa

SEQ ID NO: 478 - DNA, 753 bp, Artificial Sequence
  MGD34 heavy chain nuc
gaggtgcagctggtggagtcgggcccaggactgatgaagacttcggggaccctgtccctcacgtgcgctgtgtctggtgactacgtcaatactaataggaggtggagttgggtccgccaggccccagggaagggcctggagtggattggagaggttcatcaaagtgggagaaccaattacaacccgtccctcaagagccgagtcaccatatcagtagacaagtctaagaatcagttctctctgaaggtggactctgtgaccgccgcggacacggccgtgtattactgtgcgagagcgtctccactcaaatctcagagggacaccgaagatctgcccagaccctccatctcggctgagccgggcaccgtgatccccctggggagccatgtgactttcgtgtgccggggcccggttggggttcaaacattccgcctggagagggagaggaattatttatacagtgatactgaagatgtgtctcaaactagtccatctgagtcggaggccagattccgcattgactcagtaaatgcaggcaatgccgggctttttcgctgcatctattacaagtcccgtaaatggtctgagcagagtgactacctggagctggtggtgaaaggtgaggacgtcacctgggccctgtcccagtctcaagtcgaccctcgagcttgtccccagggggagctccccataagtaccgatatttactacgtggacgtctggggcaacgggaccacgttcaccgtctcctca

SEQ ID NO: 479 - DNA, 321 bp, Artificial Sequence; identical to SEQ ID NO: 407
  MGD34 light chain nuc

SEQ ID NO: 480 - protein, 9 aa, Artificial Sequence
  MGD35 CDRH1 aa
GASISSINW

SEQ ID NO: 481 - protein, 7 aa, Artificial Sequence
  MGD35 CDRH2 aa
IHHNGST

SEQ ID NO: 482 - protein, 143 aa, Artificial Sequence
  MGD35 CDRH3 aa
ATASSLKSQRDTNLPRPSLSAEPGTVIPLGSPVTFVCRGPVGVHTFRLERAGRSTYNDTEDVSHPSPSESEARFRIDSVSEGNAGPYRCVYYKSSKWSEESYCLDLLVKTEDVTWARPQPQLDPRACPQGDLRISTDFYYMDV

SEQ ID NO: 483 - protein, 6 aa, Artificial Sequence
  MGD35 CDRL1 aa
QAIGTY

SEQ ID NO: 484 - skipped

SEQ ID NO: 485 - protein, 9 aa, Artificial Sequence
  MGD35 CDRL2 long aa
LIYNASTLQ

SEQ ID NO: 486 - protein, 9 aa, Artificial Sequence
  MGD35 CDRL3 aa
QHYYNYPPA

SEQ ID NO: 487 - DNA, 27 bp, Artificial Sequence
  MGD35 CDRH1 nuc
ggtgcctccatcagtagtattaattgg

SEQ ID NO: 488 - DNA, 21 bp, Artificial Sequence
  MGD35 CDRH2 nuc
atccatcataatgggagcacc

SEQ ID NO: 489 - DNA, 429 bp, Artificial Sequence
  MGD35 CDRH3 nuc
gcgactgcctcttcattgaagtctcagagggacaccaatttgcccagaccctccctctcggcggagccaggcaccgtgatccccctggggagccctgtgactttcgtgtgccggggcccggttggggttcacacattccgcctggagagggcgggtagatccacatacaatgatactgaagatgtgtctcatcctagtccatctgagtcagaggccagattccgcattgactcagtgagtgagggaaatgccgggccttatcgctgcgtctattataagtcctctaaatggtccgaggagagttactgcctggacctgctggtcaaaactgaggacgtcacgtgggcccggccccagcctcagctcgaccctcgagcttgtccccagggggacctccgcattagcaccgatttttactacatggacgtc

SEQ ID NO: 490 - DNA, 18 bp, Artificial Sequence
  MGD35 CDRL1 nuc
caggctattggcacttat

SEQ ID NO: 491 - skipped

SEQ ID NO: 492 - DNA, 27 bp, Artificial Sequence
  MGD35 CDRL2 long nuc
ctgatctataatgcttccactttgcaa

SEQ ID NO: 493 - DNA, 27 bp, Artificial Sequence
  MGD35 CDRL3 nuc
caacactattataattatcctccggcc

SEQ ID NO: 494 - protein, 250 aa, Artificial Sequence
  MGD35 heavy chain aa
QVQLQESGPGLVKPSGTLSLTCAVSGASISSINWWSWVRQTPEKGLEWIGQIHHNGSTNYNPSLKSRVAISVDKSKNQFSLKLTSFTAADTAVYYCATASSLKSQRDTNLPRPSLSAEPGTVIPLGSPVTFVCRGPVGVHTFRLERAGRSTYNDTEDVSHPSPSESEARFRIDSVSEGNAGPYRCVYYKSSKWSEESYCLDLLVKTEDVTWARPQPQLDPRACPQGDLRISTDFYYMDVWGKGTTVTVSS

SEQ ID NO: 495 - protein, 107 aa, Artificial Sequence
  MGD35 light chain aa
AIRMTQSPSSLSASTGDRVTITCRTSQAIGTYLAWYQQNPGKAPNLLIYNASTLQSGVPSRFSASGSGTDFTLTISGLQSDDFVTYFCQHYYNYPPAFGQGTRLEIQ

SEQ ID NO: 496 - DNA, 750 bp, Artificial Sequence
  MGD35 heavy chain nuc
caggtgcagctgcaggagtcgggcccaggactggtgaagccttcggggaccctgtccctcacctgcgctgtctctggtgcctccatcagtagtattaattggtggagttgggtccgtcagaccccagaaaaggggctggagtggattggacaaatccatcataatgggagcaccaactacaacccgtccctcaagagtcgggtcgccatatcagttgacaagtccaagaaccagttctccctgaagttgacttctttcaccgccgcggacacggccgtgtattattgtgcgactgcctcttcattgaagtctcagagggacaccaatttgcccagaccctccctctcggcggagccaggcaccgtgatccccctggggagccctgtgactttcgtgtgccggggcccggttggggttcacacattccgcctggagagggcgggtagatccacatacaatgatactgaagatgtgtctcatcctagtccatctgagtcagaggccagattccgcattgactcagtgagtgagggaaatgccgggccttatcgctgcgtctattataagtcctctaaatggtccgaggagagttactgcctggacctgctggtcaaaactgaggacgtcacgtgggcccggccccagcctcagctcgaccctcgagcttgtccccagggggacctccgcattagcaccgatttttactacatggacgtctggggcaaagggaccacggtcaccgtctcttca

SEQ ID NO: 497 - DNA, 321 bp, Artificial Sequence
  MGD35 light chain nuc
gccatccggatgacccagtctccatcctcactctctgcatctacgggagacagagtcaccatcacttgtcggacgagtcaggctattggcacttatttagcgtggtatcagcagaacccagggaaagcccctaacctcctgatctataatgcttccactttgcaaagtggggtcccatcaaggttcagcgccagtggctctgggacagatttcactctcaccatcagcggcctgcagtctgacgattttgtcacttatttctgccaacactattataattatcctccggccttcggccaagggacacgactggagattcaa

SEQ ID NO: 498 - protein, 9 aa, Artificial Sequence
  MGD39 CDRH1 aa
GGSISAYRW

SEQ ID NO: 499 - protein, 7 aa, Artificial Sequence
  MGD39 CDRH2 aa
VYNDGNT

SEQ ID NO: 500 - protein, 144 aa, Artificial Sequence
  MGD39 CDRH3 aa
ATISPLRPQSDTDDLPRPSISAEPGTVIPLGSHVTFVCRGPIGVQTFRLERERRSLYSDTEDVSQVSPFASEARFRIDSVSEGNAGPYRCIYYKDRKWSDQSDYLELLVKGEDVTWALPQSQLAPRACPQEELNISTDIFSMNV

SEQ ID NO: 501 - protein, 6 aa, Artificial Sequence
  MGD39 CDRL1 aa
HDVGNY

SEQ ID NO: 502 - skipped

SEQ ID NO: 503 - protein, 9 aa, Artificial Sequence
  MGD39 CDRL2 long aa
LIHGASTLQ

SEQ ID NO: 504 - protein, 9 aa, Artificial Sequence
  MGD39 CDRL3 aa
QQYYSSPPG

SEQ ID NO: 505 - DNA, 27 bp, Artificial Sequence
  MGD39 CDRH1 nuc
ggtggctccatcagtgcttataggtgg

SEQ ID NO: 506 - DNA, 21 bp, Artificial Sequence
  MGD39 CDRH2 nuc
gtctataatgatggcaatacc

SEQ ID NO: 507 - DNA, 432 bp, Artificial Sequence
  MGD39 CDRH3 nuc
gcgacaatttctccactgaggcctcagagtgacaccgacgatctgcccagaccctccatctcggctgagccaggcaccgtgatccccctggggagccatgtgaccttcgtgtgccggggcccaattggggttcaaacattccgcctggagagggagagaagatccttatacagtgatactgaagatgtgtctcaagttagtccatttgcgtcagaggccagattccgcattgactcagtaagtgaaggaaatgccgggccatatcgctgcatctattataaggaccggaaatggtctgaccagagtgactacctggagttgctggtgaaaggtgaggacgtcacctgggccctgccccagtctcagctcgcccctcgcgcttgtccccaggaagaattgaacattagtaccgatattttctccatgaacgtc

SEQ ID NO: 508 - DNA, 18 bp, Artificial Sequence
  MGD39 CDRL1 nuc
catgatgttggtaattat

SEQ ID NO: 509 - skipped

SEQ ID NO: 510 - DNA, 27 bp, Artificial Sequence
  MGD39 CDRL2 long nuc
ctgatccacggtgcgtccactttgcaa

SEQ ID NO: 511 - DNA, 27 bp, Artificial Sequence
  MGD39 CDRL3 nuc
caacaatattacagttcccctccgggc

SEQ ID NO: 512 - protein, 251 aa, Artificial Sequence
  MGD39 heavy chain aa
QVRLQESGPGLVKPSGTLSLTCTVSGGSISAYRWWSWVRQAPGKGLEWIGQVYNDGNTNYNPSLKGRVAMSVDKSKNRFSLRLASVTAADTAVYYCATISPLRPQSDTDDLPRPSISAEPGTVIPLGSHVTFVCRGPIGVQTFRLERERRSLYSDTEDVSQVSPFASEARFRIDSVSEGNAGPYRCIYYKDRKWSDQSDYLELLVKGEDVTWALPQSQLAPRACPQEELNISTDIFSMNVWGKGTTVTVSS

SEQ ID NO: 513 - protein, 107 aa, Artificial Sequence
  MGD39 light chain aa
AIRMTQSPASLSASIGDRVTITCRTSHDVGNYLDWYQQKPGKAPKLLIHGASTLQTGVPSRFSGSGAGTDFTLNITCLQSGDFAMYYCQQYYSSPPGFGQGTRLEIK

SEQ ID NO: 514 - DNA, 753 bp, Artificial Sequence
  MGD39 heavy chain nuc
caggtgcggctgcaggagtcgggcccaggactggtgaagccttcggggaccctgtccctcacctgcactgtctctggtggctccatcagtgcttataggtggtggagttgggtccgccaggccccaggcaagggcctggagtggattggacaggtctataatgatggcaataccaactacaacccgtccctcaagggtcgagtcgccatgtcagtggacaagtccaagaatcgattttccctgagattagcgtctgtgaccgccgcggacacggccgtgtattactgtgcgacaatttctccactgaggcctcagagtgacaccgacgatctgcccagaccctccatctcggctgagccaggcaccgtgatccccctggggagccatgtgaccttcgtgtgccggggcccaattggggttcaaacattccgcctggagagggagagaagatccttatacagtgatactgaagatgtgtctcaagttagtccatttgcgtcagaggccagattccgcattgactcagtaagtgaaggaaatgccgggccatatcgctgcatctattataaggaccggaaatggtctgaccagagtgactacctggagttgctggtgaaaggtgaggacgtcacctgggccctgccccagtctcagctcgcccctcgcgcttgtccccaggaagaattgaacattagtaccgatattttctccatgaacgtctggggcaaagggaccacggtcaccgtctcctca

SEQ ID NO: 515 - DNA, 321 bp, Artificial Sequence
  MGD39 light chain nuc
gccatccggatgacccagtctccagcgtctctgtctgcatctataggagacagagtcaccatcacttgtcggacgagtcatgatgttggtaattatttagattggtatcaacaaaaaccaggaaaagcccctaaactcctgatccacggtgcgtccactttgcaaactggggtcccatcacggttcagcggcagtggagccgggacagatttcactctcaacatcacctgcctgcagtctggagatttcgcaatgtattattgtcaacaatattacagttcccctccgggcttcggccaagggacacgactggagattaaa

SEQ ID NO: 516 - protein, 9 aa, Artificial Sequence
  MGD41 CDRH1 aa
GGSINTDKW

SEQ ID NO: 517 - protein, 7 aa, Artificial Sequence
  MGD41 CDRH2 aa
VLHTGST

SEQ ID NO: 518 - protein, 144 aa, Artificial Sequence
  MGD41 CDRH3 aa
ATISTLRPQRDIEDLPRPSLSAEPGTVVPLGSHVTFVCRGPVGVQTFRLERESRSTYNDTEDVSQPSPFESEARFRIDSVSEGNAGPYRCIYYKSPKWSDQSDYVELLVKGEDVTWAPPQSQLAPRACPQGELRTSTDIFSMNV

SEQ ID NO: 519 - protein, 6 aa, Artificial Sequence
  MGD41 CDRL1 aa
QDIGNY

SEQ ID NO: 520 - skipped

SEQ ID NO: 521 - protein, 9 aa, Artificial Sequence
  MGD41 CDRL2 long aa
LIHGASTLL

SEQ ID NO: 522 - protein, 9 aa, Artificial Sequence
  MGD41 CDRL3 aa
LQYYSSPPA

SEQ ID NO: 523 - DNA, 27 bp, Artificial Sequence
  MGD41 CDRH1 nuc
ggtggctccatcaacactgataagtgg

SEQ ID NO: 524 - DNA, 21 bp, Artificial Sequence
  MGD41 CDRH2 nuc
gtccttcatactgggagcacc

SEQ ID NO: 525 - DNA, 432 bp, Artificial Sequence
  MGD41 CDRH3 nuc
gcgactatttctacattgaggcctcagcgggacatcgaagatctgcccagaccctccctctcggctgagccaggcaccgtggtccccctggggagccatgtgactttcgtgtgccggggcccggttggggttcaaacattccgcctggagagggagagcagatccacatacaatgatactgaagatgtgtctcaacctagtccatttgagtcagaggccagatttcgcattgactcagtaagtgaaggaaatgccgggccttatcgctgcatctattataagtcccctaaatggtctgaccagagtgactacgtggagttgctggtgaaaggtgaggacgtcacctgggccccgccccagtctcagctcgcccctcgagcttgtccccagggagaactccgcactagcaccgatattttctccatgaacgtc

SEQ ID NO: 526 - DNA, 18 bp, Artificial Sequence
  MGD41 CDRL1 nuc
caggatattggtaattac

SEQ ID NO: 527 - skipped

SEQ ID NO: 528 - DNA, 27 bp, Artificial Sequence
  MGD41 CDRL2 long nuc
ctgatccatggtgcatccactttgctg

SEQ ID NO: 529 - DNA, 27 bp, Artificial Sequence
  MGD41 CDRL3 nuc
ctacaatattacagttcccctccggcc

SEQ ID NO: 530 - protein, 251 aa, Artificial Sequence
  MGD41 heavy chain aa
EVQLVESGPGLVKPSGTLSVTCTISGGSINTDKWWTWVRQPPGKGLEWVGEVLHTGSTNYNPSLRGRVTISVDKSKNQFSLRLSSVTAADTAVYYCATISTLRPQRDIEDLPRPSLSAEPGTVVPLGSHVTFVCRGPVGVQTFRLERESRSTYNDTEDVSQPSPFESEARFRIDSVSEGNAGPYRCIYYKSPKWSDQSDYVELLVKGEDVTWAPPQSQLAPRACPQGELRTSTDIFSMNVWGKGTTVTVSS

SEQ ID NO: 531 - protein, 107 aa, Artificial Sequence
  MGD41 light chain aa
AIRMTQSPSSLSAFTGDRVTISCRASQDIGNYLDWYHQKPGRAPKLLIHGASTLLTGVPSRFSGSGSGTDFTLNITCLQSGDFGIYYCLQYYSSPPAFGPGTRLEIK

SEQ ID NO: 532 - DNA, 753 bp, Artificial Sequence
  MGD41 heavy chain nuc
gaggtgcagctggtggagtcgggcccaggactggtgaagccttcggggaccctgtccgtcacctgcactatctctggtggctccatcaacactgataagtggtggacttgggtccgccagcccccagggaagggccttgagtgggtaggggaagtccttcatactgggagcaccaactacaacccgtccctgaggggtcgagtcaccatatcagtggacaagtccaagaaccagttctccctgaggctgagttctgtgaccgccgcggacacggccgtatattattgtgcgactatttctacattgaggcctcagcgggacatcgaagatctgcccagaccctccctctcggctgagccaggcaccgtggtccccctggggagccatgtgactttcgtgtgccggggcccggttggggttcaaacattccgcctggagagggagagcagatccacatacaatgatactgaagatgtgtctcaacctagtccatttgagtcagaggccagatttcgcattgactcagtaagtgaaggaaatgccgggccttatcgctgcatctattataagtcccctaaatggtctgaccagagtgactacgtggagttgctggtgaaaggtgaggacgtcacctgggccccgccccagtctcagctcgcccctcgagcttgtccccagggagaactccgcactagcaccgatattttctccatgaacgtctggggcaaagggaccacggtcaccgtctcctca

SEQ ID NO: 533 - DNA, 322 bp, Artificial Sequence
  MGD41 light chain nuc
gccatccggatgacccagtctccatcctcactgtctgcatttacaggagacagagtcaccatctcttgccgggcgagtcaggatattggtaattacttagattggtatcaccaaaagccaggaagagcccctaagctcctgatccatggtgcatccactttgctgactggggtcccatcacgattcagcggcagtggatccggaacagatttcactctcaacatcacctgcctgcagtctggagattttggaatttattactgtctacaatattacagttcccctccggccttcggcccagggacacggctggagattaaga

SEQ ID NO: 534 - protein, 9 aa, Artificial Sequence
  MGD47 CDRH1 aa
GGSISGYKW

SEQ ID NO: 535 - protein, 7 aa, Artificial Sequence
  MGD47 CDRH2 aa
VYDDGDT

SEQ ID NO: 536 - protein, 144 aa, Artificial Sequence
  MGD47 CDRH3 aa
ATISPLRPQSDTGDLPRPSISAEPGTAIPLGSQVTFVCRGPIGVQTFRLERESRALYNDSEDVSQVSPSASEARFRIDSVSEGNAGPYRCIYYKARRWSDQSDYLELLVKGEDVTWALPQSQLAPRACPQEDLNISTDIFSTNV

SEQ ID NO: 537 - protein, 6 aa, Artificial Sequence
  MGD47 CDRL1 aa
QDVGNY

SEQ ID NO: 538 - skipped

SEQ ID NO: 539 - protein, 9 aa, Artificial Sequence
  MGD47 CDRL2 long aa
LIHGASTLQ

SEQ ID NO: 540 - protein, 9 aa, Artificial Sequence
  MGD47 CDRL3 aa
QQYYTSPPV

SEQ ID NO: 541 - DNA, 27 bp, Artificial Sequence
  MGD47 CDRH1 nuc
ggtggctccatcagtggttacaagtgg

SEQ ID NO: 542 - DNA, 21 bp, Artificial Sequence
  MGD47 CDRH2 nuc
gtctatgatgatggcgacacc

SEQ ID NO: 543 - DNA, 432 bp, Artificial Sequence
  MGD47 CDRH3 nuc
gcgacaatttctccactgaggcctcagagtgacaccggagatctgcccagaccctccatctcggctgagccaggcaccgcgatccccctggggagccaagtgactttcgtgtgccggggcccaattggggttcaaacattccgcctggagagggagagtcgcgccttatataatgattctgaagatgtgtctcaagttagtccatctgcgtcagaggccagattccgcattgactcagtaagtgaaggcaatgccgggccttatcgctgtatctattataaggcccgcagatggtctgaccagagtgactatttggagttgttggtgaaaggtgaggacgtcacctgggccctgccccagtctcagctcgcccctcgcgcttgtccccaggaagatttgaacattagtaccgatattttctctacgaacgtc

SEQ ID NO: 544 - DNA, 18 bp, Artificial Sequence
  MGD47 CDRL1 nuc
caggatgttggaaattat

SEQ ID NO: 545 - skipped

SEQ ID NO: 546 - DNA, 27 bp, Artificial Sequence
  MGD47 CDRL2 long nuc
ctcatccacggtgcgtccactttgcaa

SEQ ID NO: 547 - DNA, 27 bp, Artificial Sequence
  MGD47 CDRL3 nuc
caacaatattacacttcccctccggtc

SEQ ID NO: 548 - protein, 251 aa, Artificial Sequence
  MGD47 heavy chain aa
QVRLQESGPGLVKPSGTLSLTCTVSGGSISGYKWWSWVRQAPGKGLEWIGQVYDDGDTNYNPDLKGRVALSVDKSKSRFSLSLASVTAADTAIYFCATISPLRPQSDTGDLPRPSISAEPGTAIPLGSQVTFVCRGPIGVQTFRLERESRALYNDSEDVSQVSPSASEARFRIDSVSEGNAGPYRCIYYKARRWSDQSDYLELLVKGEDVTWALPQSQLAPRACPQEDLNISTDIFSTNVWGKGTTVTVSS

SEQ ID NO: 549 - protein, 107 aa, Artificial Sequence
  MGD47 light chain aa
AIRMTQSPASLSASVGDRVTITCRTSQDVGNYLDWYQQKPGKAPKLLIHGASTLQAGVPSRFNGSGSGTDFTLGISCVQSGDFAIYYCQQYYTSPPVFGQGTRLEIK

SEQ ID NO: 550 - DNA, 753 bp, Artificial Sequence
  MGD47 heavy chain nuc
caggtgcggctgcaggagtcgggcccaggactggtgaagccttcggggaccctgtccctcacctgcactgtctcgggtggctccatcagtggttacaagtggtggagttgggtccgccaggccccaggcaagggcctggagtggattggacaggtctatgatgatggcgacaccaactacaatccggacctgaagggtcgagtcgccctgtcagtggacaagtccaagagtcgattttccctcagcctagcgtctgtgaccgccgcggacacggccatatacttctgtgcgacaatttctccactgaggcctcagagtgacaccggagatctgcccagaccctccatctcggctgagccaggcaccgcgatccccctggggagccaagtgactttcgtgtgccggggcccaattggggttcaaacattccgcctggagagggagagtcgcgccttatataatgattctgaagatgtgtctcaagttagtccatctgcgtcagaggccagattccgcattgactcagtaagtgaaggcaatgccgggccttatcgctgtatctattataaggcccgcagatggtctgaccagagtgactatttggagttgttggtgaaaggtgaggacgtcacctgggccctgccccagtctcagctcgcccctcgcgcttgtccccaggaagatttgaacattagtaccgatattttctctacgaacgtctggggcaaagggacaacggtcaccgtctcttca

SEQ ID NO: 551 - DNA, 321 bp, Artificial Sequence
  MGD47 light chain nuc
gccatccggatgacccagtctccagcgtccctgtctgcatctgtaggagacagagtcaccatcacttgtcggacgagtcaggatgttggaaattatttagattggtatcaacaaaaaccaggaaaagcccctaaactcctcatccacggtgcgtccactttgcaagctggggtcccatcacgtttcaacggcagtggatccgggacagatttcactctcggcatcagttgtgtgcagtctggagatttcgcgatctattactgtcaacaatattacacttcccctccggtcttcggccaagggacacgactggagattaaa

SEQ ID NO: 552 - protein, 9 aa, Artificial Sequence
  MGD55 CDRH1 aa
GGSISAYKW

SEQ ID NO: 553 - protein, 7 aa, Artificial Sequence
  MGD55 CDRH2 aa
VYHNGNT

SEQ ID NO: 554 - protein, 144 aa, Artificial Sequence
  MGD55 CDRH3 aa
ATISPLRPQSDTDDLPRPSISAEPGTVIPLGSHVTFVCRGPIGVQTFRLERESRSLYSDTEDVSQVSPFASEARFRIDSVSEGNAGPYRCIYYKDRKWSDQSDYLELLVKGEDVTWALPQSQLAPRACPQEELNISTDIFSMNV

SEQ ID NO: 555 - protein, 6 aa, Artificial Sequence
  MGD55 CDRL1 aa
QDVGNY

SEQ ID NO: 556 - skipped

SEQ ID NO: 557 - protein, 9 aa, Artificial Sequence
  MGD55 CDRL2 long aa
LIHGASTLQ

SEQ ID NO: 558 - protein, 9 aa, Artificial Sequence
  MGD55 CDRL3 aa
QQYYSSPPG

SEQ ID NO: 559 - DNA, 27 bp, Artificial Sequence
  MGD55 CDRH1 nuc
ggtggctccatcagtgcttataagtgg

SEQ ID NO: 560 - DNA, 21 bp, Artificial Sequence
  MGD55 CDRH2 nuc
gtctatcataatggcaacacc

SEQ ID NO: 561 - DNA, 432 bp, Artificial Sequence
  MGD55 CDRH3 nuc
gcgacaatttctccactgaggcctcagagtgacaccgacgatctgcccagaccctccatctcggctgagccaggcaccgtgatccccctggggagccatgtgactttcgtgtgccggggcccaattggggttcaaacattccgcctggagagggagagtagatccttatacagtgatactgaagatgtgtctcaagttagtccatttgcgtcagaggccagattccgcattgactcagtaagtgaaggaaatgccgggccatatcgctgcatctattataaggaccggaaatggtctgaccagagtgactacctggagttgctggtgaaaggtgaggacgtcacctgggccctgccccagtctcagctcgcccctcgcgcttgtccccaggaagaattgaacattagtaccgatattttctccatgaacgtc

SEQ ID NO: 562 - DNA, 18 bp, Artificial Sequence
  MGD55 CDRL1 nuc
caggatgttggtaattat

SEQ ID NO: 563 - skipped

SEQ ID NO: 564 - DNA, 27 bp, Artificial Sequence
  MGD55 CDRL2 long nuc
ctgatccacggtgcgtccactttgcaa

SEQ ID NO: 565 - DNA, 27 bp, Artificial Sequence
  MGD55 CDRL3 nuc
caacaatattacagttcccctccgggc

SEQ ID NO: 566 - protein, 251 aa, Artificial Sequence
  MGD55 heavy chain aa
QVQLQESGPGLVKPSGTLSLTCTVSGGSISAYKWWSWVRQAPGKGLEWIGQVYHNGNTNYNPSLKGRVAMSVDKSKNRFSLRLASVTAADTAVYYCATISPLRPQSDTDDLPRPSISAEPGTVIPLGSHVTFVCRGPIGVQTFRLERESRSLYSDTEDVSQVSPFASEARFRIDSVSEGNAGPYRCIYYKDRKWSDQSDYLELLVKGEDVTWALPQSQLAPRACPQEELNISTDIFSMNVWGKGTTVTVSS

SEQ ID NO: 567 - protein, 107 aa, Artificial Sequence
  MGD55 light chain aa
AIRMTQSPASLSASIGDRVTITCRTSQDVGNYLDWYQQKPGKAPKLLIHGASTLQTGVPSRFSGSGAGTDFTLNITCLQSGDFAMYYCQQYYSSPPGFGQGTRLEIK

SEQ ID NO: 568 - DNA, 753 bp, Artificial Sequence
  MGD55 heavy chain nuc
caggtgcagctgcaggagtcgggcccaggactggtgaagccttcggggaccctgtccctcacctgcactgtctctggtggctccatcagtgcttataagtggtggagttgggtccgccaggccccaggcaagggcctggagtggattggacaggtctatcataatggcaacaccaactacaacccgtccctcaagggtcgagtcgccatgtcagtggacaagtccaagaatcgattttccctgagactagcgtctgtgaccgccgcggacacggccgtgtattactgtgcgacaatttctccactgaggcctcagagtgacaccgacgatctgcccagaccctccatctcggctgagccaggcaccgtgatccccctggggagccatgtgactttcgtgtgccggggcccaattggggttcaaacattccgcctggagagggagagtagatccttatacagtgatactgaagatgtgtctcaagttagtccatttgcgtcagaggccagattccgcattgactcagtaagtgaaggaaatgccgggccatatcgctgcatctattataaggaccggaaatggtctgaccagagtgactacctggagttgctggtgaaaggtgaggacgtcacctgggccctgccccagtctcagctcgcccctcgcgcttgtccccaggaagaattgaacattagtaccgatattttctccatgaacgtctggggcaaagggaccacggtcaccgtctcctca

SEQ ID NO: 569 - DNA, 322 bp, Artificial Sequence
  MGD55 light chain nuc
gccatccggatgacccagtctccagcgtctctgtctgcatctataggagacagagtcaccatcacttgtcggacgagtcaggatgttggtaattatttagattggtatcaacaaaaaccaggaaaagcccctaaactcctgatccacggtgcgtccactttgcaaactggggtcccatcacggttcagcggcagtggagccgggacagatttcactctcaacatcacctgcctgcagtctggagatttcgcaatgtattactgtcaacaatattacagttcccctccgggcttcggccaagggacacgactggaaattaaga

SEQ ID NO: 570 - protein, 9 aa, Artificial Sequence
  MGD56 CDRH1 aa
GGSITTNNW

SEQ ID NO: 571 - protein, 7 aa, Artificial Sequence
  MGD56 CDRH2 aa
IFRSGTT

SEQ ID NO: 572 - protein, 144 aa, Artificial Sequence
  MGD56 CDRH3 aa
ATASPFKSQRDTKDLPRPSLSAEPGTVIPLGSHVTFVCRGPVGVQTFRLQRESRSLYNDTEDVSHPSPSESEARFRIDSVSEGNAGPYRCVYYKSSKWSEESDCLELLVKTEDVTWARPQPQLDPRACPRGDLRISTDVYYMDV

SEQ ID NO: 573 - protein, 6 aa, Artificial Sequence
  MGD56 CDRL1 aa
QAITSY

SEQ ID NO: 574 - skipped

SEQ ID NO: 575 - protein, 9 aa, Artificial Sequence
  MGD56 CDRL2 long aa
LIYNASTLQ

SEQ ID NO: 576 - protein, 9 aa, Artificial Sequence
  MGD56 CDRL3 aa
QHYYTYPPA

SEQ ID NO: 577 - DNA, 27 bp, Artificial Sequence
  MGD56 CDRH1 nuc
ggtggctccatcactactaataattgg

SEQ ID NO: 578 - DNA, 21 bp, Artificial Sequence
  MGD56 CDRH2 nuc
atctttcgtagtgggaccacc

SEQ ID NO: 579 - DNA, 432 bp, Artificial Sequence
  MGD56 CDRH3 nuc
gcgacagcctctccattcaagtctcagagggacaccaaagatttgcccagaccctccctctcggctgagccaggcaccgtgatccccctggggagtcatgtgactttcgtgtgccggggcccggttggggttcagacattccgcctgcagagggagagtagatccctttacaatgatactgaagatgtgtctcatcctagtccatctgagtcagaggccagattccgcattgactcagtgagtgagggaaatgccgggccttatcgctgcgtctattataagtcctctaaatggtccgaggagagtgactgcctggagctgctggtcaaaactgaggacgtcacctgggcccggccccagcctcagctcgaccctcgagcttgtccccggggggacctccgcattagcaccgatgtttactacatggacgtc

SEQ ID NO: 580 - DNA, 18 bp, Artificial Sequence
  MGD56 CDRL1 nuc
caggctattaccagttat

SEQ ID NO: 581 - skipped

SEQ ID NO: 582 - DNA, 27 bp, Artificial Sequence
  MGD56 CDRL2 long nuc
ctgatctataatgcttccactttgcaa

SEQ ID NO: 583 - DNA, 27 bp, Artificial Sequence
  MGD56 CDRL3 nuc
caacactattatacttaccctccggcc

SEQ ID NO: 584 - protein, 251 aa, Artificial Sequence
  MGD56 heavy chain aa
QVQLQESGPGLVKPSGTLSLTCAVSGGSITTNNWWSWVRQTPGKGLEWIGEIFRSGTTNYNPSLKSRVAISLDKSKNQFSLKLTSVTAADTAVYYCATASPFKSQRDTKDLPRPSLSAEPGTVIPLGSHVTFVCRGPVGVQTFRLQRESRSLYNDTEDVSHPSPSESEARFRIDSVSEGNAGPYRCVYYKSSKWSEESDCLELLVKTEDVTWARPQPQLDPRACPRGDLRISTDVYYMDVWGKGTTVTVSS

SEQ ID NO: 585 - protein, 107 aa, Artificial Sequence
  MGD56 light chain aa
AIRMTQSPSSLSASTGDRVTITCRASQAITSYLAWYRQKPGKAPDLLIYNASTLQSGVPSRFSASGSGTDFALTITGLQSEDFVIYFCQHYYTYPPAFGQGTRLEIK

SEQ ID NO: 586 - DNA, 753 bp, Artificial Sequence
  MGD56 heavy chain nuc
caggtgcagctgcaggagtcgggcccaggactggtgaagccttcggggaccctgtccctcacctgcgctgtctctggtggctccatcactactaataattggtggagttgggtccgtcagaccccaggaaaggggctggagtggattggagaaatctttcgtagtgggaccaccaactacaacccgtccctcaagagtcgggtcgccatttcattagacaagtccaagaaccagttctccctgaagttgacttctgtgaccgccgcggacacggccgtgtattactgtgcgacagcctctccattcaagtctcagagggacaccaaagatttgcccagaccctccctctcggctgagccaggcaccgtgatccccctggggagtcatgtgactttcgtgtgccggggcccggttggggttcagacattccgcctgcagagggagagtagatccctttacaatgatactgaagatgtgtctcatcctagtccatctgagtcagaggccagattccgcattgactcagtgagtgagggaaatgccgggccttatcgctgcgtctattataagtcctctaaatggtccgaggagagtgactgcctggagctgctggtcaaaactgaggacgtcacctgggcccggccccagcctcagctcgaccctcgagcttgtccccggggggacctccgcattagcaccgatgtttactacatggacgtctggggcaaagggaccacggtcaccgtctcctca

SEQ ID NO: 587 - DNA, 321 bp, Artificial Sequence
  MGD56 light chain nuc
gccatccggatgacccagtctccatcctcactctctgcatctacaggggacagagtcaccatcacttgtcgggcgagtcaggctattaccagttatttagcctggtatcggcagaaaccagggaaagcccctgacctcctgatctataatgcttccactttgcaaagtggggtcccatcaagattcagcgccagtggctctgggacagatttcgctctcaccatcaccggcctgcagtctgaggattttgtaatttatttctgccaacactattatacttaccctccggccttcggccaagggacacgactggagattaaa

SEQ ID NO: 588 - protein, 330 aa, Artificial Sequence
  IgG1 CH1-CH2-CH3 aa
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 589 - protein, 107 aa, Artificial Sequence [sequence omitted]

SEQ ID NO: 590 - protein, 106 aa, Artificial Sequence
  IgG CL aa
GQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 591 - DNA, 990 bp, Artificial Sequence
  IgG1 CH1-CH2-CH3 nucl
gcgtcgaccaagggcccatcggtcttccccctggcaccctcctccaagagcacctctgggggcacagcggccctgggctgcctggtcaaggactacttccccgaacctgtgacggtctcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaa

SEQ ID NO: 592 - DNA, 321 bp, Artificial Sequence
  IgG CK nucl
cgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 593 - DNA, 318 bp, Artificial Sequence
  IgG CL nucl
ggtcagcccaaggctgccccctcggtcactctgttcccgccctcctctgaggagcttcaagccaacaaggccacactggtgtgtctcataagtgacttctacccgggagccgtgacagtggcttggaaagcagatagcagccccgtcaaggcgggagtggagaccaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctacagaatgttca

SEQ ID NO: 594 - protein, 250 aa, Artificial Sequence
  FI499V_DexinDJ aa
QVQPVQSGAEVKEPGSSVKVSCKTSGGLIRKSAVSWVRQAPGQGLEWMGGISALFNTKDYAEKFQGRLTITADESTATAYMELSSLTSEDTAIYYCATASPLKSQRDTDLPRPSISAEPGTVIPLGSHVTFVCRGPVGVQTFRLERERNYLYSDTEDVSQTSPSESEARFRIDSVNAGNAGLFRCIYYKSRKWSEQSDYLELVVKGEDVTWALSQSQDDPRACPQGELPISTDIYYVDVWGNGTTVTVSS

SEQ ID NO: 595 - DNA, 750 bp, Artificial Sequence
  FI499V_DexinDJ nucl
caggtgcagcccgtccagtctggagcagaggtgaaggaacctggcagctccgtgaaggtctcttgcaaaacaagtggcgggctgatccgcaaaagtgccgtgtcatgggtccgacaggctcctggacagggactggaatggatgggaggcatcagcgcactgttcaacactaaggactacgccgaaaaatttcagggccggctgactattaccgccgatgagagtacagccactgcttatatggaactgtctagtctgaccagcgaggacacagctatctactattgcgcaaccgcctcaccactgaagtcccagagagacaccgacctgccaagaccttccatctctgcagaacctggcacagtgattccactggggtcccacgtgactttcgtctgtaggggaccagtgggcgtccagacctttcgcctggagcgggaaagaaattacctgtattccgacactgaggacgtgagccagaccagtccctcagagagcgaagctaggttccgcatcgattccgtgaacgctgggaatgcaggactgtttagatgcatctactataagtctaggaaatggagcgagcagtccgactacctggaactggtggtcaaaggggaggatgtgacatgggctctgtcccagtctcaggacgatccaagagcatgtccccagggcgagctgcccatctctactgacatctactatgtggatgtctggggcaacgggaccacagtgaccgtctcaagc

SEQ ID NO: 596 - protein, 248 aa, Artificial Sequence
  FI499VJ_DexinD aa
QVQPVQSGAEVKEPGSSVKVSCKTSGGLIRKSAVSWVRQAPGQGLEWMGGISALFNTKDYAEKFQGRLTITADESTATAYMELSSLTSEDTAIYYCATASPLKSQRDTDLPRPSISAEPGTVIPLGSHVTFVCRGPVGVQTFRLERERNYLYSDTEDVSQTSPSESEARFRIDSVNAGNAGLFRCIYYKSRKWSEQSDYLELVVKGEDVTWALSQSQDDPRACPQGELPISTDIFDYWGQGTLVTVSS

SEQ ID NO: 597 - DNA, 744 bp, Artificial Sequence
  FI499VJ_DexinD nucl
caggtgcagcccgtccagtctggagcagaggtgaaggaacctggcagctccgtgaaggtctcttgcaaaacaagtggcgggctgatccgcaaaagtgccgtgtcatgggtccgacaggctcctggacagggactggaatggatgggaggcatcagcgcactgttcaacactaaggactacgccgaaaaatttcagggccggctgaccattacagccgatgagagtactgccaccgcttatatggaactgtctagtctgaccagcgaggacacagctatctactattgcgcaaccgcctcaccactgaagtcccagagagacaccgacctgccaagaccttccatctctgcagaacctggcacagtgattccactggggtcccacgtgactttcgtctgtaggggaccagtgggcgtccagacctttcgcctggagcgggaaagaaattacctgtattccgacactgaggacgtgagccagaccagtccctcagagagcgaagctaggttccgcatcgattccgtgaacgctgggaatgcaggactgtttagatgcatctactataagtctaggaaatggagcgagcagtccgactacctggaactggtggtcaaaggggaggatgtgacttgggctctgtcccagtctcaggacgatccaagagcatgtccccagggcgagctgcccatctctaccgacattttcgattattggggccaggggacactggtgactgtctcaagc

SEQ ID NO: 598 - protein, 249 aa, Artificial Sequence
  MGD21_exin_longGS aa
EVQLVETGPGLMKTSGTLSLTCAVSGDYVNTNRRWSWVRQAPGKGLEWIGEVHQSGRTNYNPSLKSRVTISVDKSKNQFSLKVDSVTAADTAVYYCARGGGGSGGGGSDLPRPSISAEPGTVIPLGSHVTFVCRGPVGVQTFRLERERNYLYSDTEDVSQTSPSESEARFRIDSVNAGNAGLFRCIYYKSRKWSEQSDYLELVVKGEDVTWALGGGGSGGGGSGGGGSGGGGSYYVDVWGNGTTVTVSS

SEQ ID NO: 599 - DNA, 747 bp, Artificial Sequence
  MGD21_exin_longGS nucl
gaagtgcagctggtggaaaccggccctggactgatgaagacctctggcacactgagtctgacatgcgctgtgagtggggactacgtcaacactaatcggagatggtcttgggtgcgacaggcaccaggaaaaggactggagtggatcggggaagtgcaccagagcggaaggaccaactataatcctagcctgaagtcccgcgtgacaatttcagtcgataagagcaaaaaccagttctccctgaaagtggactctgtcactgccgctgataccgcagtgtactattgtgccagaggcgggggaggctctgggggaggcgggagtgacctgcccaggcctagcatctccgctgaaccagggactgtgattcccctgggatctcacgtgaccttcgtctgcagaggccctgtgggggtccagacatttcgcctggagcgggaaagaaactacctgtattctgacaccgaggatgtgagtcagacatctcccagtgagtcagaagcaaggttccgcatcgattccgtcaacgccggaaatgctggcctgtttcgatgtatctactataagagccggaaatggagcgagcagtccgactacctggaactggtggtcaagggcgaggatgtgacctgggccctgggcgggggaggctctgggggaggcgggagtggaggcgggggatcaggtggaggcgggtcgtactatgtggacgtgtggggcaacgggaccacagtgaccgtcagctcc

SEQ ID NO: 600 - protein, 229 aa, Artificial Sequence
  MGD21_exin_short GS aa
EVQLVETGPGLMKTSGTLSLTCAVSGDYVNTNRRWSWVRQAPGKGLEWIGEVHQSGRTNYNPSLKSRVTISVDKSKNQFSLKVDSVTAADTAVYYCARGGGGSDLPRPSISAEPGTVIPLGSHVTFVCRGPVGVQTFRLERERNYLYSDTEDVSQTSPSESEARFRIDSVNAGNAGLFRCIYYKSRKWSEQSDYLELVVKGEDVTWALGGGGSYYVDVWGNGTTVTVSS

SEQ ID NO: 601 - DNA, 687 bp, Artificial Sequence
  MGD21_exin_short GS nucl
gaagtgcagctggtggaaaccggccctggactgatgaagacctctggcacactgagtctgacatgcgctgtgagtggggactacgtcaacactaatcggagatggtcttgggtgcgacaggcaccaggaaaaggactggagtggatcggggaagtgcaccagagcggaaggaccaactataatcctagcctgaagtcccgcgtgacaatttcagtcgataagagcaaaaaccagttctccctgaaagtggactctgtcactgccgctgataccgcagtgtactattgtgccagagggggaggcgggagtgacctgcccaggcctagcatctccgctgaaccagggactgtgattcccctgggatctcacgtgaccttcgtctgcagaggccctgtgggggtccagacatttcgcctggagcgggaaagaaactacctgtattctgacaccgaggatgtgagtcagacatctcccagtgagtcagaagcaaggttccgcatcgattccgtcaacgccggaaatgctggcctgtttcgatgtatctactataagagccggaaatggagcgagcagtccgactacctggaactggtggtcaagggcgaggatgtgacctgggccctgggaggcgggggatcatactatgtggacgtgtggggcaacgggaccacagtgaccgtcagctcc

SEQ ID NO: 602 - protein, 133 aa, Artificial Sequence
  MGD21_NOexin aa
EVQLVETGPGLMKTSGTLSLTCAVSGDYVNTNRRWSWVRQAPGKGLEWIGEVHQSGRTNYNPSLKSRVTISVDKSKNQFSLKVDSVTAADTAVYYCARASPLKSQRDTGELPISTDIYYVDVWGNGTTVTVSS

SEQ ID NO: 603 - DNA, 399 bp, Artificial Sequence
  MGD21_NOexin nucl
gaagtgcagctggtggaaaccggccctggactgatgaagacttcaggaaccctgagcctgacttgtgccgtgagcggcgactacgtcaacaccaatcggagatggagttgggtgcggcaggcaccaggaaaaggcctggagtggatcggcgaagtgcaccagtctgggcgaacaaactataatccctctctgaagagtagagtgactatttccgtggacaagtctaaaaaccagttcagcctgaaagtggactccgtcacagccgctgatactgccgtgtactattgtgcaagggccagtcccctgaagtcacagcgcgataccggggagctgcctatcagcacagacatctactatgtggatgtctgggggaatggaaccacagtgacagtcagctcc

SEQ ID NO: 604 - protein, 230 aa, Artificial Sequence
  MGD21_NOin aa
EVQLVETGPGLMKTSGTLSLTCAVSGDYVNTNRRWSWVRQAPGKGLEWIGEVHQSGRTNYNPSLKSRVTISVDKSKNQFSLKVDSVTAADTAVYYCARASPLKSQRDTDLPRPSISAEPGTVIPLGSHVTFVCRGPVGVQTFRLERERNYLYSDTEDVSQTSPSESEARFRIDSVNAGNAGLFRCIYYKSRKWSEQSDYLELVVKGELPISTDIYYVDVWGNGTTVTVSS

SEQ ID NO: 605 - DNA, 690 bp, Artificial Sequence
  MGD21_NOin nucl
gaggtgcagctggtcgaaaccggcccagggctgatgaagacttccggaaccctgtctctgacatgcgccgtgtccggggactacgtcaacactaatcggagatggtcttgggtgaggcaggctcctggaaaaggcctggagtggatcggggaagtgcaccagtccggacggaccaactataatccatctctgaagagtagagtgacaattagtgtcgataagtcaaaaaaccagttctctctgaaagtggacagtgtcacagccgctgatactgcagtgtactattgtgcaagagcaagccccctgaagtcccagagagacaccgacctgcccaggccttctatcagtgctgaaccaggcactgtgattcccctggggtctcatgtgaccttcgtctgtagaggccccgtgggagtccagacttttcgcctggagagggaacgcaattacctgtattcagacaccgaggatgtgagccagacatcacctagcgagtccgaagcccgattccggatcgacagtgtgaacgctggaaatgcaggcctgtttcgctgtatctactataagagccgaaaatggtcagagcagagcgattacctggaactggtggtcaaaggcgagctgcctatcagcactgacatctactatgtggatgtctgggggaacggaaccacagtgaccgtcagctcc

SEQ ID NO: 606 - protein, 142 aa, Artificial Sequence
  MGD21_NOVD aa
DLPRPSISAEPGTVIPLGSHVTFVCRGPVGVQTFRLERERNYLYSDTEDVSQTSPSESEARFRIDSVNAGNAGLFRCIYYKSRKWSEQSDYLELVVKGEDVTWALSQSQDDPRACPQGELPISTDIYYVDVWGNGTTVTVSS

SEQ ID NO: 607 - DNA, 426 bp, Artificial Sequence
  MGD21_NOVD nucl
gacctgccacgaccatctatttccgccgaacctgggactgtcattcctctggggagccacgtcacatttgtctgccggggacctgtcggggtgcagactttccggctggagcgggaaagaaactacctgtattctgacaccgaagatgtgagtcagacaagcccatccgagtctgaagctaggttccgcatcgactccgtcaacgccggcaatgctgggctgtttcgatgcatctactataagagcagaaaatggagcgagcagtccgactacctggaactggtggtcaagggagaggatgtcacctgggcactgagtcagtcacaggacgatccccgggcctgtcctcagggcgagctgcccatcagcactgatatctactatgtggatgtctgggggaatggcactactgtgaccgtctcaagc

SEQ ID NO: 608 - protein, 250 aa, Artificial Sequence
  MGD21GL_exinWT aa
QVQLQESGPGLVKPSGTLSLTCAVSGGSISSSNWWSWVRQPPGKGLEWIGEIYHSGSTNYNPSLKSRVTISVDKSKNQFSLKLSSVTAADTAVYYCARASPLKSQRDTDLPRPSISAEPGTVIPLGSHVTFVCRGPVGVQTFRLERERNYLYSDTEDVSQTSPSESEARFRIDSVNAGNAGLFRCIYYKSRKWSEQSDYLELVVKGEDVTWALSQSQDDPRACPQGELPISTDIYYMDVWGKGTTVTVSS

SEQ ID NO: 609 - DNA, 750 bp, Artificial Sequence
  MGD21GL_exinWT nucl
caggtccagctgcaggaaagcggcccaggactggtgaagcctagcggaacactgagtctgacttgtgccgtgagcggagggagcatcagctcctctaactggtggtcttgggtgaggcagccccctggcaagggactggagtggatcggcgaaatctaccacagcgggtccaccaactataatccttcactgaagagccgcgtgacaatcagtgtggacaagtcaaaaaatcagttcagcctgaaactgagttcagtgaccgccgctgatacagcagtctactattgcgcacgggccagcccactgaaatcccagcgagacactgatctgccacggccctctatcagtgctgaacccggaacagtgattcctctgggctcccatgtgactttcgtctgtcgcggaccagtgggcgtccagacctttcgactggagcgggaaagaaactacctgtattctgacactgaggatgtgagtcagacctcacccagcgagtccgaagccaggttccgcatcgacagcgtcaacgctgggaatgcaggactgtttagatgcatctactataagtccaggaaatggtccgagcagtctgactacctggaactggtggtcaagggggaggatgtgacatgggccctgtctcagagtcaggacgatcctagagcttgtccacagggcgagctgcccatttcaaccgatatctattacatggatgtctggggcaagggcaccaccgtgaccgtgagcagc

SEQ ID NO: 610 - protein, 251 aa, Artificial Sequence
  MGD21_wholeGL aa
QVQLQESGPGLVKPSGTLSLTCAVSGGSISSSNWWSWVRQPPGKGLEWIGEIYHSGSTNYNPSLKSRVTISVDKSKNQFSLKLSSVTAADTAVYYCARASPLKSQRDTEDLPRPSISAEPGTVIPLGSHVTFVCRGPVGVQTFRLERESRSTYNDTEDVSQASPSESEARFRIDSVSEGNAGPYRCIYYKPPKWSEQSDYLELLVKGEDVTWALPQSQLDPRACPQGELPISTDIYYMDVWGKGTTVTVSS

SEQ ID NO: 611 - DNA, 767 bp, Artificial Sequence
  MGD21_wholeGL nucl
caggtgcagctgcaggaaagcggaccaggcctggtcaagccctcaggcactctgagcctgacctgcgctgtgagtggcgggtcaatcagctcctctaattggtggtcctgggtgaggcagccccctgggaaaggactggagtggatcggcgaaatctaccactctgggagtacaaactataatcccagcctgaagtcccgcgtgactatttccgtggacaagtctaaaaatcagttcagcctgaaactgagttcagtgacagccgctgatactgcagtctactattgcgcacgagccagtcctctgaagtcccagcgggacactgaggacctgcctagaccatcaatcagcgccgagcctggaactgtgattccactgggctctcatgtgaccttcgtctgtagaggaccagtgggagtccagaccttccggctggagagagaatcccgatctacctacaacgacacagaagatgtgagccaggctagtccatcagagagcgaagcacggtttagaatcgactccgtgtctgaggggaatgccggaccctacagatgcatctactataagccacccaaatggtctgagcagagtgactatctggaactgctggtgaaaggagaggatgtcacctgggcactgcctcagtctcagctggaccccagagcttgtcctcagggagagctgcctatcagcaccgacatctactatatggacgtgtggggcaaagggaccacagtgacagtcagctccgcgtcgacttcgca

SEQ ID NO: 612 - protein, 153 aa, Artificial Sequence
  MGD21_NOex aa
EVQLVETGPGLMKTSGTLSLTCAVSGDYVNTNRRWSWVRQAPGKGLEWIGEVHQSGRTNYNPSLKSRVTISVDKSKNQFSLKVDSVTAADTAVYYCARASPLKSQRDTGEDVTWALSQSQDDPRACPQGELPISTDIYYVDVWGNGTTVTVSS

SEQ ID NO: 613 - DNA, 459 bp, Artificial Sequence
  MGD21_NOex nucl
gaagtgcagctggtggaaaccggccctggactgatgaagacttccggaaccctgtctctgacttgcgccgtgtctggcgactacgtcaacaccaatcggagatggagctgggtgcggcaggctccaggaaaaggcctggagtggatcggcgaagtgcaccagtccgggcgaacaaactataatccctcactgaagagcagagtgactattagtgtcgataagtcaaaaaaccagttctctctgaaagtggacagtgtcacagccgctgatactgccgtgtactattgcgcaagggccagccctctgaagtcccagagagacaccggggaggatgtgacatgggctctgtctcagagtcaggacgatccccgggcatgtcctcagggcgaactgccaatcagcaccgacatctactatgtggatgtctgggggaatggaaccacagtgacagtcagctcc

SEQ ID NO: 614 - protein, 243 aa, Artificial Sequence
  mIgG2b aa
AMVRSPSGPISTINPCPPCKECHKCPAPNLEGGPSVFIFPPNIKDVLMISLTPKVTCVVVDVSEDDPDVQISWFVNNVEVHTAQTQTHREDYNSTIRVVSTLPIQHQDWMSGKEFKCKVNNKDLPSPIERTISKIKGLVRAPQVYILPPPAEQLSRKDVSLTCLVVGFNPGDISVEWTSNGHTEENYKDTAPVLDSDGSYFIYSKLNMKTSKWEKTDSFSCNVRHEGLKNYYLKKTISRSPGK

SEQ ID NO: 615 - DNA, 729 bp, Artificial Sequence
  mIgG2b nucl
gccatggttagatctcccagcgggcccatttcaacaatcaacccctgtcctccatgcaaggagtgtcacaaatgcccagctcctaacctcgagggtggaccatccgtcttcatcttccctccaaatatcaaggatgtactcatgatctccctgacacccaaggtcacgtgtgtggtggtggatgtgagcgaggatgacccagacgtccagatcagctggtttgtgaacaacgtggaagtacacacagctcagacacaaacccatagagaggattacaacagtactatccgggtggtcagcaccctccccatccagcaccaggactggatgagtggcaaggagttcaaatgcaaggtcaacaacaaagacctcccatcacccatcgagagaaccatctcaaaaattaaagggctagtcagagctccacaagtatacatcttgccgccaccagcagagcagttgtccaggaaagatgtcagtctcacttgcctggtcgtgggcttcaaccctggagacatcagtgtggagtggaccagcaatgggcatacagaggagaactacaaggacaccgcaccagtcctggactctgacggttcttacttcatatatagcaagctcaatatgaaaacaagcaagtgggagaaaacagattccttctcatgcaacgtgagacacgagggtctgaaaaattactacctgaagaagaccatctcccggtctccgggtaaa

SEQ ID NO: 616 - protein, 232 aa, Artificial Sequence
  hIgG1 aa
AMVRSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 617 - DNA, 696 bp, Artificial Sequence
  hIgG1 nucl
gccatggttagatctgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 618 - protein, 152 aa, Artificial Sequence
  DexinDJ variable part aa
ASPLKSQRDTDLPRPSISAEPGTVIPLGSHVTFVCRGPVGVQTFRLERERNYLYSDTEDVSQTSPSESEARFRIDSVNAGNAGLFRCIYYKSRKWSEQSDYLELVVKGEDVTWALSQSQDDPRACPQGELPISTDIYYVDVWGNGTTVTVSS

SEQ ID NO: 619 - DNA, 456 bp, Artificial Sequence
  DexinDJ variable part nucl
gcgtctccactcaaatctcagagggacaccgatctgcccagaccctccatctcggctgagccgggcaccgtgatccccctggggagccatgtgactttcgtgtgccggggcccggttggggttcaaacattccgcctggagagggagaggaattatttatacagtgatactgaagatgtgtctcaaactagtccatctgagtcggaggccagattccgcattgactcagtaaatgcaggcaatgccgggctttttcgctgcatctattacaagtcccgtaaatggtctgagcagagtgactacctggagctggtggtgaaaggtgaggacgtcacctgggccctgtcccagtctcaagacgaccctcgagcttgtccccagggggagctccccataagtaccgatatttactacgtggacgtctggggcaacgggaccacggtcaccgtctcctca

SEQ ID NO: 620 - protein, 395 aa, Artificial Sequence
  DexinDJ-mIgG2b complete sequence aa
ASPLKSQRDTDLPRPSISAEPGTVIPLGSHVTFVCRGPVGVQTFRLERERNYLYSDTEDVSQTSPSESEARFRIDSVNAGNAGLFRCIYYKSRKWSEQSDYLELVVKGEDVTWALSQSQDDPRACPQGELPISTDIYYVDVWGNGTTVTVSSAMVRSPSGPISTINPCPPCKECHKCPAPNLEGGPSVFIFPPNIKDVLMISLTPKVTCVVVDVSEDDPDVQISWFVNNVEVHTAQTQTHREDYNSTIRVVSTLPIQHQDWMSGKEFKCKVNNKDLPSPIERTISKIKGLVRAPQVYILPPPAEQLSRKDVSLTCLVVGFNPGDISVEWTSNGHTEENYKDTAPVLDSDGSYFIYSKLNMKTSKWEKTDSFSCNVRHEGLKNYYLKKTISRSPGK

SEQ ID NO: 621 - DNA, 1185 bp, Artificial Sequence
  DexinDJ-mIgG2b complete sequence  nucl
gcgtctccactcaaatctcagagggacaccgatctgcccagaccctccatctcggctgagccgggcaccgtgatccccctggggagccatgtgactttcgtgtgccggggcccggttggggttcaaacattccgcctggagagggagaggaattatttatacagtgatactgaagatgtgtctcaaactagtccatctgagtcggaggccagattccgcattgactcagtaaatgcaggcaatgccgggctttttcgctgcatctattacaagtcccgtaaatggtctgagcagagtgactacctggagctggtggtgaaaggtgaggacgtcacctgggccctgtcccagtctcaagacgaccctcgagcttgtccccagggggagctccccataagtaccgatatttactacgtggacgtctggggcaacgggaccacggtcaccgtctcctcagccatggttagatctcccagcgggcccatttcaacaatcaacccctgtcctccatgcaaggagtgtcacaaatgcccagctcctaacctcgagggtggaccatccgtcttcatcttccctccaaatatcaaggatgtactcatgatctccctgacacccaaggtcacgtgtgtggtggtggatgtgagcgaggatgacccagacgtccagatcagctggtttgtgaacaacgtggaagtacacacagctcagacacaaacccatagagaggattacaacagtactatccgggtggtcagcaccctccccatccagcaccaggactggatgagtggcaaggagttcaaatgcaaggtcaacaacaaagacctcccatcacccatcgagagaaccatctcaaaaattaaagggctagtcagagctccacaagtatacatcttgccgccaccagcagagcagttgtccaggaaagatgtcagtctcacttgcctggtcgtgggcttcaaccctggagacatcagtgtggagtggaccagcaatgggcatacagaggagaactacaaggacaccgcaccagtcctggactctgacggttcttacttcatatatagcaagctcaatatgaaaacaagcaagtgggagaaaacagattccttctcatgcaacgtgagacacgagggtctgaaaaattactacctgaagaagaccatctcccggtctccgggtaaa

SEQ ID NO: 622 - protein, 384 aa, Artificial Sequence
  DexinDJ-hIgG1 complete sequence aa
ASPLKSQRDTDLPRPSISAEPGTVIPLGSHVTFVCRGPVGVQTFRLERERNYLYSDTEDVSQTSPSESEARFRIDSVNAGNAGLFRCIYYKSRKWSEQSDYLELVVKGEDVTWALSQSQDDPRACPQGELPISTDIYYVDVWGNGTTVTVSSAMVRSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 623 - DNA, 1152 bp, Artificial Sequence
  DexinDJ-hIgG1 complete sequence  nucl
gcgtctccactcaaatctcagagggacaccgatctgcccagaccctccatctcggctgagccgggcaccgtgatccccctggggagccatgtgactttcgtgtgccggggcccggttggggttcaaacattccgcctggagagggagaggaattatttatacagtgatactgaagatgtgtctcaaactagtccatctgagtcggaggccagattccgcattgactcagtaaatgcaggcaatgccgggctttttcgctgcatctattacaagtcccgtaaatggtctgagcagagtgactacctggagctggtggtgaaaggtgaggacgtcacctgggccctgtcccagtctcaagacgaccctcgagcttgtccccagggggagctccccataagtaccgatatttactacgtggacgtctggggcaacgggaccacggtcaccgtctcctcagccatggttagatctgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 624 - protein, 142 aa, Artificial Sequence; identical to SEQ ID NO: 606
  exinDJ variable part aa

SEQ ID NO: 625 - DNA, 426 bp, Artificial Sequence
  exinDJ variable part nucl
gatctgcccagaccctccatctcggctgagccgggcaccgtgatccccctggggagccatgtgactttcgtgtgccggggcccggttggggttcaaacattccgcctggagagggagaggaattatttatacagtgatactgaagatgtgtctcaaactagtccatctgagtcggaggccagattccgcattgactcagtaaatgcaggcaatgccgggctttttcgctgcatctattacaagtcccgtaaatggtctgagcagagtgactacctggagctggtggtgaaaggtgaggacgtcacctgggccctgtcccagtctcaagacgaccctcgagcttgtccccagggggagctccccataagtaccgatatttactacgtggacgtctggggcaacgggaccacggtcaccgtctcctca

SEQ ID NO: 626 - protein, 374 aa, Artificial Sequence
  exinDJ-mIgG2b complete sequence aa
DLPRPSISAEPGTVIPLGSHVTFVCRGPVGVQTFRLERERNYLYSDTEDVSQTSPSESEARFRIDSVNAGNAGLFRCIYYKSRKWSEQSDYLELVVKGEDVTWALSQSQDDPRACPQGELPISTDIYYVDVWGNGTTVTVSSAMVRSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 627 - DNA, 1122 bp, Artificial Sequence
  exinDJ-mIgG2b complete sequence  nucl
gatctgcccagaccctccatctcggctgagccgggcaccgtgatccccctggggagccatgtgactttcgtgtgccggggcccggttggggttcaaacattccgcctggagagggagaggaattatttatacagtgatactgaagatgtgtctcaaactagtccatctgagtcggaggccagattccgcattgactcagtaaatgcaggcaatgccgggctttttcgctgcatctattacaagtcccgtaaatggtctgagcagagtgactacctggagctggtggtgaaaggtgaggacgtcacctgggccctgtcccagtctcaagacgaccctcgagcttgtccccagggggagctccccataagtaccgatatttactacgtggacgtctggggcaacgggaccacggtcaccgtctcctcagccatggttagatctgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 628 - protein, 105 aa, Artificial Sequence
  exin variable part aa
DLPRPSISAEPGTVIPLGSHVTFVCRGPVGVQTFRLERERNYLYSDTEDVSQTSPSESEARFRIDSVNAGNAGLFRCIYYKSRKWSEQSDYLELVVKGEDVTWAL

SEQ ID NO: 629 - DNA, 315 bp, Artificial Sequence
  exin variable part nucl
gatctgcccagaccctccatctcggctgagccgggcaccgtgatccccctggggagccatgtgactttcgtgtgccggggcccggttggggttcaaacattccgcctggagagggagaggaattatttatacagtgatactgaagatgtgtctcaaactagtccatctgagtcggaggccagattccgcattgactcagtaaatgcaggcaatgccgggctttttcgctgcatctattacaagtcccgtaaatggtctgagcagagtgactacctggagctggtggtgaaaggtgaggacgtcacctgggccctg

SEQ ID NO: 630 - protein, 337 aa, Artificial Sequence
  exin-mIgG2b complete sequence aa
DLPRPSISAEPGTVIPLGSHVTFVCRGPVGVQTFRLERERNYLYSDTEDVSQTSPSESEARFRIDSVNAGNAGLFRCIYYKSRKWSEQSDYLELVVKGEDVTWALAMVRSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 631 - DNA, 1011 bp, Artificial Sequence
  exin-mIgG2b complete sequence  nucl
gatctgcccagaccctccatctcggctgagccgggcaccgtgatccccctggggagccatgtgactttcgtgtgccggggcccggttggggttcaaacattccgcctggagagggagaggaattatttatacagtgatactgaagatgtgtctcaaactagtccatctgagtcggaggccagattccgcattgactcagtaaatgcaggcaatgccgggctttttcgctgcatctattacaagtcccgtaaatggtctgagcagagtgactacctggagctggtggtgaaaggtgaggacgtcacctgggccctggccatggttagatctgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 632 - protein, 97 aa, Artificial Sequence; identical to SEQ ID NO: 83
  exon variable part aa

SEQ ID NO: 633 - DNA, 291 bp, Artificial Sequence; identical to SEQ ID NO: 84
  exon variable part nucl

SEQ ID NO: 634 - protein, 340 aa, Artificial Sequence
  ex-mIgG2b complete sequence aa
DLPRPSISAEPGTVIPLGSHVTFVCRGPVGVQTFRLERERNYLYSDTEDVSQTSPSESEARFRIDSVNAGNAGLFRCIYYKSRKWSEQSDYLELVVKAMVRSPSGPISTINPCPPCKECHKCPAPNLEGGPSVFIFPPNIKDVLMISLTPKVTCVVVDVSEDDPDVQISWFVNNVEVHTAQTQTHREDYNSTIRVVSTLPIQHQDWMSGKEFKCKVNNKDLPSPIERTISKIKGLVRAPQVYILPPPAEQLSRKDVSLTCLVVGFNPGDISVEWTSNGHTEENYKDTAPVLDSDGSYFIYSKLNMKTSKWEKTDSFSCNVRHEGLKNYYLKKTISRSPGK

SEQ ID NO: 635 - DNA, 1020 bp, Artificial Sequence
  ex-mIgG2b complete sequence  nucl
gatctgcccagaccctccatctcggctgagccgggcaccgtgatccccctggggagccatgtgactttcgtgtgccggggcccggttggggttcaaacattccgcctggagagggagaggaattatttatacagtgatactgaagatgtgtctcaaactagtccatctgagtcggaggccagattccgcattgactcagtaaatgcaggcaatgccgggctttttcgctgcatctattacaagtcccgtaaatggtctgagcagagtgactacctggagctggtggtgaaagccatggttagatctcccagcgggcccatttcaacaatcaacccctgtcctccatgcaaggagtgtcacaaatgcccagctcctaacctcgagggtggaccatccgtcttcatcttccctccaaatatcaaggatgtactcatgatctccctgacacccaaggtcacgtgtgtggtggtggatgtgagcgaggatgacccagacgtccagatcagctggtttgtgaacaacgtggaagtacacacagctcagacacaaacccatagagaggattacaacagtactatccgggtggtcagcaccctccccatccagcaccaggactggatgagtggcaaggagttcaaatgcaaggtcaacaacaaagacctcccatcacccatcgagagaaccatctcaaaaattaaagggctagtcagagctccacaagtatacatcttgccgccaccagcagagcagttgtccaggaaagatgtcagtctcacttgcctggtcgtgggcttcaaccctggagacatcagtgtggagtggaccagcaatgggcatacagaggagaactacaaggacaccgcaccagtcctggactctgacggttcttacttcatatatagcaagctcaatatgaaaacaagcaagtgggagaaaacagattccttctcatgcaacgtgagacacgagggtctgaaaaattactacctgaagaagaccatctcccggtctccgggtaaa

SEQ ID NO: 636 - protein, 329 aa, Artificial Sequence
  ex-hIgG1 complete sequence aa
DLPRPSISAEPGTVIPLGSHVTFVCRGPVGVQTFRLERERNYLYSDTEDVSQTSPSESEARFRIDSVNAGNAGLFRCIYYKSRKWSEQSDYLELVVKAMVRSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 637 - DNA, 987 bp, Artificial Sequence
  ex-hIgG1 complete sequence  nucl
gatctgcccagaccctccatctcggctgagccgggcaccgtgatccccctggggagccatgtgactttcgtgtgccggggcccggttggggttcaaacattccgcctggagagggagaggaattatttatacagtgatactgaagatgtgtctcaaactagtccatctgagtcggaggccagattccgcattgactcagtaaatgcaggcaatgccgggctttttcgctgcatctattacaagtcccgtaaatggtctgagcagagtgactacctggagctggtggtgaaagccatggttagatctgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 638 - DNA, 294 bp, Artificial Sequence
  LAIR1ex nucl
gaggacctgccaagacccagcatctccgcagaacctgggactgtgattccactgggctcccacgtgaccttcgtctgcagaggccccgtgggagtccagaccttccggctggagcgcgaatctcgaagtacctacaacgacacagaggacgtgagccaggcctcacccagcgagtccgaagctcggttcagaatcgactctgtcagtgaaggaaatgccggcccttacagatgcatctactataagccccctaaatggtcagagcagagcgattatctggaactgctggtgaag

SEQ ID NO: 639 - protein, 167 aa, Plasmodium falciparum
IAALAVNAWKTTALKNAIAAAQKAGDAAGKIAGESKGVETIIGILEQYYSIYELKGTPLKSFFATTHYTDISNIATVIDTELNTSCGLNSLANQAICGLRTKLGLVAKPGQVMVTQKEAITKMITNVVHKSEITAEAAKTEVAATKTAAAIKMNTEAIEAATTPYYT

SEQ ID NO: 640 - protein, 117 aa, Plasmodium falciparum
CELYSPTNYDSDPEMKRVMQQFVDRTTQRFHEYDESLQSKRKQCKDQCDKEIQKIILKDKIEKEFTEKLSTLQTDITTKDIPTCVCEKSLADKMEKVCLKCAQNLGGIVAPSTGVLG

SEQ ID NO: 641 - protein, 166 aa, Plasmodium falciparum
IGQLGLDAWKAAALVTAKELAEKAGAAAGLKAGDIHGMKIVIEGLKALKVDTLKSGIFNSFVNNSHYTEVTGLAIAIDTEMNEVCSATYIGIHPICVVREKLGVIPKAGGTMVKQKDAITNVLKQALEKATQSAEALSETTAEDVAAKLTAQKTGAINTIFMSNQT

SEQ ID NO: 642 - protein, 117 aa, Plasmodium falciparum
CELYSPTNYDSDPEMKRVMQQFHDRTTQRFHEYDERMKTTRQECKEQCDKEIQKIILKDRLEKELMDKFATLHTDIQSDAIPTCVCEKSLADKTEKFCLNCGVQLGGGVLQASGLLG